SEQUENCE LISTING (609 sequences)

SEQ ID NO: 1 - DNA, 214000 bp, Homo sapiens
gactaagatgaatatgcattcattcaccaaaatctcatattcccaaaaagcaggaaaggtagtacagtgagatggatgatgccttcacatgactcagatgtcacgtgtttctcaccattgagacccccaaggcaccccctcccagcatttaccagaatgtgtgtgtaactatttacagtgatttgtgtaattatttgattgtttctcttgtatcctgtagcaatgagggtagagattatatcccacctaccactgcagctccaggatccagcttcacaaacatttgttgaatgaatgaataagaaaagaggacacccccaaagaggctgcaagggaaaaagctacaaagacagaagcaccaggaaaaagtagggtcatgtaagtcaaagcaggaaaaaagttccatggtggggtggtcagcagtgtctaatgccacgaaggcacaaagtaggataaaggttaaaaatcagcctttggttttggcaaatatgaagcttatcggtagccttagcgagaacaattccatcagggagcagaagctaactgcagtgggttgagtcatcaagcaggcataaggaagtagggataccccattataagctactctttcaagaagctcaaatctgaaggttaggagaattaggtcagtagctagaaggaaatgtggagtcgaggggctgtttttcctcccaaggagtataaaggtgtaacgttgcatgaaaccacttcagacaaaggccgatatcaatagagaagttaaaacgcacgcctcaagatttgggaaggcttggggttgggcttaaagaggtaggagcatatttcctatcctaggacagagaataaagaagaaaggataggttcccatggagataaatttctaagtgttaaagaagaggctcagaaaattctagcatgataggctcacttttttctttttccatgaaggagatggcaaagtcaactgacatgagaaaggtgacaatactgatgggttgaagagcgatggacatttgaaataacttcttagaccagtagaggctggagttcataaatcagaactggctacaggttatatatgtttttttttttttctccaacagcataagataacagagcgaagtctgtagaaatgaaagaagagtcagatgaggatagctggagctagtgcaaggagggaagcaccacggtgggagccaggtaccccctggatttataattcatactgaattccaacaacagaagggctctaagcaggagagtgacagatttcagaagactgagacacatttggtaaaaaaaagtaggaggaaaacctgattctggaattagggcagccaatagacggcagtattttcagaaaggagggaatggtcaacagtgactttctagtctggagctcaggaggaagaggcaactctacctgatggtattaagatcatggaggtagctgagatcacctagcttgtgtgtgtcaaatgagaaaagaagaaagaataggagaagttccccaggaacacagacattaagtggggctgtggtgacaacacaagaagagaggcttgcaaaggagcctgagcagctgtcatgagagaggtaggatggtggactcggagaagaggcagaagatgttcttaaaggaaggacactgctgccaagtagtcagccaattggtgacaaagaaagaccctgttgcgagaaaaaaagtcagtgaagtagtaggaacgatgacagatgacactgggttgaagactgaggagagagaagtgtaagagtggaagcagagggcagaccactcttctgagacactgaagaggcatagttagaaataaaggggagtcgccagaaaggaatttgtggctaagcaagaggttttctttaagactgaaatacataagcatgatttaaatgctgctgggatggagttcacagacctggaagacagaagacaaagcggatcatcaagatagtggaatttactgaaatgagagaggaaaatcccatccacaggaaatgcagacatgagggaggggccagaaggacagtgaaaacatcagcaactggtcccccaacttctgagtgaatgtggagatataatcaggtaaaggactgcatcatctccctggttaatgatggagtcagagaaaagagtgtcttatacagaagttgtgatatacttggccgggcgcagtggctcacgcctgtaatctaagcactttgggaggccaaggcaggcggatcacctgaggtcaggagttcatgactggcctggtcaacatggcaaaatcccacctctactaaaaacaaaagcctgtaatcccagctactagggaggctgaggcaggagaatcgcttgaacccaggaggcagaggttgcagtgagccaaggtcgcaccactgtactccagcctgggcaacagagctagactcagtctcaaaaaaaaaaaaaaaagatgtatttattctcactgtataaatttctgtgtaagaaatactctctcatatagaagtaaatttatatataaaattatatagaaccactataaaatactcaggtttataaaatttatatataaacttgttgacatataaaattccatgtaaatgactataaagtactcttatatgaaaagtatatgaattaaattatatatcaacttacttttatattacagtatttttgttatacagaagtttatatagtgacaataaatatttctcaagaacgatttcacataatagaagtataaattatccatttccaatagtgaaaaagaaaagcagttccacaccagtgacagggctacgaatctaagaggtacaaagacttcattcttagagacactgaggtcagggcatggccaacacatctgaagctgatagaattggcgctgggttggttggagacggtacggtattactattacaatggcagacgcttggccttgataactagccaatcagggggaaagattctggtttcctctgttattatctgaactagtgtgttcccaaagggttaagatggtttatggaaggcacaagatcagcaaaccataaaggattagcactaagaaggaaggaagtagaccaagtgttaatggcgatgccatgtaagagccaggtctgcgatgtatgttctacatggtttggggggtaaaaaaaatgtcagcctccagagcacagggctttaagcctcaagtactgttaacagtagagtttactagtctacagcaggaattacaaccagtaattctaaggccaattactcaggcaagttttactagaacaaggaagctctgcttcgaggtcaaatcgatttctgcatttatagaagcatctagatgttctctgttcaaacaatggggtaaaatccccacacattttatttctgacagagtgttccctatattgcctggccaggagtgataacattgcttggctattattaataaaacattgctgtggctgggcgcagtggctcacacctgtaatcctggcactttgggaggctgaggcaggaggatcacttaactccaggagtttgacagcagcctgggcaacatagcaagatcccatctctctaaaaaattttaaaattagctgggtgtggtggcagacacctgtagtcccagctcctcaggaagctgaggtgggaggatcacttgagcccaagcaggttgaggctgcagcgtgctgtgactgtgccactgcactccagcctgcgcaacacactgagagagactctgtctcaaaaaaatacatcaaataaaaattaaaagcccatttctttcttttggtacattacagccatgcacttcaaaggctagcacaattatttttctgcagttctatatttagattctagttagaagtaacctaggaccttcatgttagaggtgtctttggcaaaactgttatgtgagtgaaacgtttaatcaattgaggataaagatgcctcattgctaatgaagatgtggtttaaggattttatgcacccagttcatttattaacaacttgtttaagctttattagctgggtctctactttataactgtgttctttaatttacaagacaataaaaattaaaatggtaaatgggaaacctatcttgcttttcaataaataatttattttaataacttcgtgggcatggtggccaaaacattttagctgtgaaaataatttcaattcatatttttttggaatcaatattaaaaggtgatatattctcaaatgaaaagtggacaaatgatcagttataggacatgattaagaaactaaccatgagccacgtgcagtggctcatgcctgtaatcccagcactctgggaggccgcggtgagcggattgcttgagcccaggagttcaagaccaggctgggcaacatggcaaaaacccggctctactaaaaatgcaaaaaaaaaaaaaaaaaaaaaaatttagctgggttttggtggcttatgcctgcagtcccagctactcgggaggctgactcgggaggctgaggcacaagaatcatttgaacccaggaggcagaggttgcaatgagctgagaatacaccactgcactccagcctgggcaacagagagagagagactcagtctcaaaaaacaaacaaacaaacaaacaaaccgctgccctgtgcttggagagatctgtttacctttaccactaaagactgttggaagtaaattttagaaggtttataatacctaaaagtaatcacttctgtcttatgaaaggttctgctgagatttttctattgtggccactagtggcaatattccagaagtcatatttaaagaatatctttagtggattcagcagtttttcaaatatgtacttttatctctccaacattcatgattgcaatttttcaaattaacctcatgatataaacaactgtactctatgatgcctcatagtacagaaactggaggcagaaagagaagttgaatgtctaagaatcggtaattctaaaactcaacatagaccattcagcattagtggttctaacaatcccactgcaaaatgagttgataatgtgtaacactttagtgaactaaagcataaagaaccatggtctcctaatgcagcaaattaaaacacatgatagctacaattaatgaagtacatagtcctggctgggcactatggtacgtcctttacatagattatctcttaaattattaaccccgttttagagatgagaacattcgggctcaggaaggttatgtaagttatataaaaatcacaaaataagagacagagctaagatttgaatccaagtgtgaccaggttcatatcaagcttccatttttgaatttatattagaggtcaataactcacctttgtccttttaaaataatttttggctctgtgacctacacaggcaagctgttatttacaaacaacccacacatctagatggtcactgtctcaccgcccacttttaccatcaggactcctagtgagctgtcaaggggaatgctataattttggaggttctaaatctgagggcttaagaaagaaagaaattgtaaaaagcaggcattactcaggggcatagattgtcaggcagatctgtcatgcttataggtaacctcccagggccaaaaatatatgtgcccaaactgcctaaatatttcctgtcacttcataatactgcctgaaatcctgccaaattagaacttcatttgtgttgcttgtcaatttttaacgcataagcaaatcacctggagatcttgttaaaatgcaaattctgattaggttaggtctgggtctgcatgtctgatatgcttccagagggcactgatgctgctggtccatggaccacacttaaagaagcaaaaaagatgtctgatatttactctctggctgcctaggagtgcttctcatttaagtgagatctctttgtgcatcataatgggagggatgagctgaaaagcagcaaattaagagtgagttaagtgtctacctcacttccctactatctgtaacaagcaggtttgggcactgtggtcaaccagaaaattctttccaggaccacaacccttgagattatgttgcaaagatgcaaggacaacttagaaataatttccagcactggtggcactggatgtctgtcagtggtgctggtggcagggtcctattcagactgtggtttacctgcctggcccgtttggttatgggccattttctgagtaccatggagcatcgcccagctgacaagggcttgtactccacccttggtgcgcagaagggaagcttggctgctactaagtttggtgcaaagtaattgtggttttgccattaatatttgatacagtgagtccctactttcctcaggtgaaactagaacttaaggggacacgctcaagttctcattatacagtactaagtttcaaaaatcagcaattttatcaaacacatgctctacagcagtggtcggcaaactttttctgtaaggggccagagagtaaatgttttagagtttctgggccacatatggtttctgttccagctataaactctgccactgtagggcaaaagcaaccctccacaatacatacatgaataggtgtgttccaaaaaaactttatttgtggaccctgaaatttgaatttcataaacttttcatgtgtcatgaaatattcttttgattttttcccaaccttttaaagatgtaacaaccatttttagcctgtaggccatatagaaacaggcagtgggctgggtttgctgacccttgctctgaagcaatgatatctcgatccaatttatacccacaaatttttctccttgaaaccatgcatttaattctcatctcttcttaccatgacaataagaagttattctatataacaaagagattgtacccacccaagccagcatttagatcatgtcatttgcttcctcaaaattttggtctttataaaaatcaattaaagcaccttaaaaggtaagcagtgatgaaatatttgaaataattggctaattaaacatcacctaaatagaaactgtgataagaaccacaaatgcgaaaaggaatcatgtagtaactaatgtggaggatatcttggtttagagatttgatgaacacgagttttgatttaaaaaaatttgtgcaatactcactgctttggtggggagcttgctatgcaagttggtagaaaaatttatcctaaagtcacagttctctaccactctggattttctcgagctaactaccattccaaactattttaggcacagttactagtttcaagaatcaggcaaattgccctggtattagcactgttctttctgtggtcacaagtcaaactactgtggtgaataaaattagatgatttctttagtctttcctttttcagcccctgtagtcaatttccagtgctccattcaaagaaaaaccaaaaatgtccagaatataaccttattttaaaacttgttaaccactgatttcacttgttaaccaaatttttttttttttttttttgagaatgaatctcactctgtcaccaggctggagtgcagtggcatgatcttggttcactgcaacctccgcctcctgggtactggttcaagcaattctcctgcctcagtctcccgagtagctgggattacaggtgtgcacccccacacccagctaatttttttgtacttttagtagagatggggtttcaccatgttggccgggctagtcttaaactcctgacctcgtgatccgcccgcctcggcctcccaaagtgctgggattgcaggcatgaaccactgcgcccagcctgttaaccaaatttctaatcacacacacttgaggcccagtaaatgcctgctgaaaagagggtgctggtggtgaggcaactgaggggctaacatactgatagctgctgaaatcttctacagctctttcttgttagaacactccatcacggctcccaggcccacaccacatgaaggaacttctagctctcttgcttgctctttacccaaatgtagttagcaagtcctgggaactaaacagcattgacacacttgaagaagacaattaggcaaatcccaactgctgtgctcctgcagctaaagatgaagactcgtccattgggcagttgattaattgtacctagaaaattaatttcaatggtcccatgacaacatacgggcagtgaagctctagtgttccccctgggtggaatcttccaggatgtatagtctcccataccagctcatcctcccatttttccagattctggttcttctctcttacctagtgtgtagtgggccaaatggtggtcccccaaaaagatatgtccatgtgttaaccctggaaactgtggatgtaaccttatttggaaaaatggggccaggtgcagtggtgtgcatgtgtagtcccagaactttgagaagccaaggtgggagaatcgttggagcccaggagttcaagaacagcccaggcaacatattgagacccccgtctctataagcaataaaaaattagctaggtgtggtggcatgcacctgaagttccagctacttgagaggctgaggcagaaggactgctcaagcccaaggagttcaaggctgcagtgagctatgatcatgtcaccccactccagcctgggtgacagagtcagactccctgtctcaggagaaaagaaaaaaaggtctttgtaaatgtaataaagaatcttgagataagatcatcctgatttaggatggaccctaaatccaatgacatttgtccttacaaaagaaaggtagagggaactgtgagacagacacagaggggagggccttgtgaagcaggaagcatagatgcagttacaagtcaaggaatgccaaggactgtctacaaccagaagccaggagagatgcatgggatgatttctccctcacagcctccagaacttctggcctccaggactgtgaagaatcaatttctgttgttttaagccaccaagtttgtgtgtcatttgttatggcaatggcagtattaggactctaatacacagtataaaaaaataaaaatagggccaggcgtggtggctcagacctataaccccagcactttgggaggctaaggcggggagatcacttgaggtcaggagtttgagaccaaccaggccaacatggtgaaaccccatctctattaaaaataaaaattagttgggcatggtggtgtgcatctgtaatcccagttactcaggaggctgaggcagaagaatcgcttgaacccaggaagtggaggttgtagtgaatgccactgcactccagcctgggtgacagagctagactccttcatcctaggacacagccaagtcttacgtagcaaaaagaagttgttaaaggtctgtagttctgcattaagcaacacaggcatgtacctatgaattatatgattataaaagtgctcggacaggcccatttcaaacttggcctctttccaccaactgtgtactgtttctcattccataactagagattatgtctttatatcctgtcaaaaaagtgaatttttgtgggctaagacattatccctgtgttaaatgcaccagtcttagtgtaaacaagcctagttcctttttcattttggctgtctagtatgcatttgtatatgctaggcagtgtactaggcaccttaaatacattaccttgtttaacctctacaggattctgggaggtaggcattatccccattttatagatgagaacactgagaagacaatgttcataagtgcgtcacttgtctgagatgacatatttactaagtagcagaaccaggcctcgagctactcagtctgatttccaaagcccctgctcttaatcacatcaacttctttcctatatcacctttcccagagtgcgctctcatggataaagagcagaagtataagttactaggcagcagaaaactgtagaggtgggaagattagataaaaaatgtaaataagaaggctttaagacaccaaaatcaaatgtaaatactttataacctgaatcagtgcttgtgttcatgaggctagaggtcgtgcattttatctctaggtctggtgatgccaatcctgatctacagccagcagcaacagttccctagcctgcctagaagtttgtaaatgcatgggctttggtaggaggaagacgagagaaagcagaacagattattacaaacccagtgcattcccccttgatgggtcaacagcgatttctttgtaagtgaaggacagcacactggttttgatgactcacgagagagtaggagggaaaaagaagtctgaggcattgcctggaagcctcgctctgcttaaacaagtacactaatggctcatgcctgttactcccagcactttggaaggccaagatgggtggatcacttgaggccaggagtttaagcccagcctggtcaacatagcgagaccttttctctattaaaaataaagaagaaagaaagtaataatgattcaagttctcattctctacaaaattcacttatgactttccaaatgctagtgaaaacttttaggtattgcaaaactgccttaatgcataacgggattctcattttacttagtctaagatgactttttcactttgaacttctgcatctttatgatcgcttagctttctgacaagcaatttcagtaagtgtttatcaatttgcatccacacgctgacacataggggtctacttacatatccttcatgtaattgagcttttgtaaatcatctttctacatggtacacttctgattttgtgtgcagctttcttgtttaagcactgtattaaatgctctgcttcctacacccttaggaacaatgagaataaaagcgtaatgttggttacttcttcatatcaaaggaagttcatctcctggttattaaaagctattattaaatggccatctttttgtgcccctgtgttaagcactctaccaagataccattaaatagataagggccacactccatagagatgatggttctatattctgtattttctgggggagttctaatttcatgcaattccttcttcttaaataaaggcaattctctaaatatattacctaatgtgctttcactttcatattcttgtaagatttttcacataaatcaattctcaaaaaatagtatcataggccttttaaaaatagtcatgttcaaaagtcaggctcatgaataaatgtgtgcattcattacatatattttcataaattcaaatttaaaagaataagagtagctagaaggtggaagaaaaatcttattctgattaggaatgcacaatcacaagaaaatttgtgatatatatagtcattttattctgtattgttttattttgattttggtaagacaagaaacaatgtagaaagtttgacaacttaaaaaagtaatatgagtgtgagaaagtcctcttccaggattagcaaaaaaatggttttttttttttttttttccgagatggagtctcgctctctcgcccaggctggagtgcagtggcgcaatcttggctcactgcaacctccgcctcccgggttcaggtgattctcttgcctcagcctcccaagtagctgggactacaggcatgtgccaccatgcccggctaattttttttatttttagtagagacggggtttcaccatgctggccaggctggtcttgaactcctgaccttgtgatctgcccgccttagcctcccaaagtgctgggattacaggcgtgagccaccgtacccagcctaaatggccaagttttattatggacaattaagctgtagaataaaaatctacttttaatagctggcatagtgcctagtggttttgaagccacaagcaggtttacaaaaaacatttaaatccatctgaatctacagaaaactaagattacctaagcagaaaatgaaaatagttcaggattaaggaagattaacaaatgaagagtatatgtattttagaagtattactttatatttttatagtataataataatatttacgttcctacacttataatgagtttcgtatatatattaaaataatttaatggattagtatgtttatatttgcttttagtaaatttggtgtatgataaactcagttgtctacattgtgagactacacctgaggcaatttctgtgttgatatatacctgaatagcagatattacttgggagcaaataaaatagcttcaggcctaattttgcaagttcatgatgggagagtaagcatgacttcaaagaactgactttgagttaaaacttgaagaatgaatgtgacaacagcaagtataaaacaatgccaggcagaggtgggactgttcatgggtatcagggtaagtgtgttgataaatgctcaaagtaggaaatacctttcttcccccacacatgtcagaaaataactgcaatagaatgcaacgacatctcagagataaagtgttcaacttagctctcagagaccgttcagttacattttgtaatgacattggaattgattgcattttgaaggcaattctaaatgcaaagtcttcattttgttgatagaagctgggttatttattatgaaatttcaaaaattaagtaaaatatctaattaggattataccagcaaaggcaaatttagaattcaagacttcatgatccatggtaagattattttaatgcaactctgctaattaactgaaatttcctttaactctcacatctgccttttacttcttaagacatttttctagtatttcaccagagcaagatatcagaagggtaaatctcttaccaatgaactttgctaattcttagtgactccgttgaccctggtgtaaggatcaggaacaaagtgaatgaaatacattttaatacatttctgctttctctaattccaaagaccactctaaagaataagttatttgtgggtattatctgaaacttgggattaaaagagaccgtgattacccttcagggattttggcaaaacttaagccatttcatctgaagagcaaagcaagcctcccacactcttggcttattctcacaattatctagatatctagcaacaaaactcttgagtagtttgttaactacagatgccaagggctgacagtttcactttcagttttcagaatatcttttgtttcagtggtgtaagcacaccatcagaatctctactatttaaaataattaagttataattgtaacttccattagatgtagtacttaaaggaatctagaagacacaactcattaattataggaatttgactgcaaattcttctggggggtctgaattgcaaaggaggcatctttgtaagtcagactcaactcattactctgtgatgcaggctcctccaaatggcagcagaaacgtattactctctagaaacactacagtagtgctacaatttcagggttctgtagagataaggacaaattgacagaaacacattcttagaaggacagtatcatttaaaataaaaatactgtcataattgtacaccaggatagcttctccataataaattctttatgattttctgatttttagaaatcagaattgaactttttaatgtgaaaaaaatgagagaattgtttcaaaataggaccacatttctgtgtataattttaaaagtttaaaaatatttgattagtagactgataaactgaaacatttttgataagcttttcattacatacaaaccatataatttgtaaaaaattggaaattattcaaaacttcacataactaaagtgaccaaataaatactggagaggaaagaaaaggagtcaaatgaatctagcattttctttttttttttttttttggagaaagggtctcactgtgccacccaggtgggagtgcaatggcacgatcatggctcactgcagcctcaactttatgggcttaggtgatcctcccacctcggcctcccaagtagcagggactacaggcatgcgccaacacgtccagctaatttttttggtattttttgcagagacgaggtttcaccaggttgccgtggctgatctggaactcctggtctcaagtgatctacccaactcagcctcccaaagtgctgggattacaggcgtgagccaccgcacccggcctaatctagcattttctaaaaggaaggacccagcagtgaacggcaatatcaataatcatgttcaagactatcagacatgcaagctggggatgaatgggtggaaggggaaaatgatgaataaatgatgaacacaagtatagacccagtggatttgagatgcccaagatgccagtgagatattcaaagtttaactcaaaagccacttcccatatgaaatcctgacaaacactcctacgtccaactggaattaatttctcttctgggctcccacagcactctgtatttttctaatagcataacactattttgtttgtagatatttctctgatagcattactatctttcctctttatcacaactgtttgaagttcttttgcctcttgcatccactgttgcccaatcccactgctggaaggctcatcttattaagttctgtattcctagtgctaacacactgtctaccatagatgatgttcaataaatggttgctaaatgaattctcttgtgataatagcactatggcaacataatcgacggtaaaaatttcttctcaatgtttacttttagcagaatgcattcatttatcaactttcattgagaatatgctaatttccatgaccctgctaggaaataggaaaataaagatgaatgtaataaggtgctcattctactgaaagtcttgactagtggagaattatggatccaacttttcatgaaatgccttcagtggtaagaattctcatatttggaataaaaaatgttatgggttgtgccaagatacctacatacttcataattttgtagagggctgtccttactgcagaaatgtatactactatagtcatatgtggaaattctttttatgatgctaactgcatgctaaccagactttttaatttaatacttgcattaaataaaccatgctaggaatccaggaatctagcttggtttattttccatacaatgtactctttgtaatatgcatatactacataaaaattctattaatggcctcgtactaaagatgtgtctgttggggaatcagttattctgtataattttatcttaattgatatattaaaatctaccaaaaatataaactccgagtaaaagtatctgcatggtgtgcatatgtttattattttaagtgtcagcgtatacattttcatgccataaagttataaaatgaaaaaatagtagccttttatattaagttcatgcttatgtagttagtaaaaacaagaaagcaattaacatacaaaccatgatggtggttaaacttgcttcagtttgtgttttttaaaatttgaaagtgagaaatacagctcgaagtcagctcatattttcagtaagtactgatgaggatgtactggccctattgactacgctgaccccattaaaatatttgtgagtctaaaggttcatatgacgctgttccttcactctagcaacaggccatacatgtcttacatagggactctgttcaattcattaatacctcctgaagtgctcaacatcgtggttcatttatagtagatactcaatacatactccattaactgaattctaagataaactgtctgttactgacagaaattttcacttaagggagtctccgtggctgaaggcaattttgaaatcctgtaaaagaacccactcctctccccaagtaatgaagtttgtcagtttcaagcctgtaataaggtactgacttaaaattaattttctaataatacagtactgctatgtatctaatgtggggttagtcaatgataggaaaaaaacataagacagagtcacatttaaaaatgtgtgcttaggtgcatggtgacacctgcctgtagtccagctattccaggggctgaggcaggaagatcccttgagctcacgagtttgaggctgcagtaagccactgcactcagcctgggcaacagagtgagaccctgtctctaaaaaaaattcgttttaagtgtgctcaggacataacaggagccgctggtaacatgccatttccactgtgaatatggtaaggacagaatccctgtctctaggccctcttccactagtcaatctcatcatcaccatcaaggccaacattggtattctctcctctgagacaaagtctttgacattttctatactatactatgtcttcctctccccaaatgcatatacaaataaaatttgaatgcttctttctccatttagtgtaattttttttataacatagacccaattttcaaaccccacaatggtggattttatttgatgtattgtaaaaagcgctggattgaagtcaaatggcttgggagacctaaattctactcctgcctgtaccatgaaagagacaaatcccaaggctttgcagggcttcagcttccttgtttgtagaataaagaattataaaatcatctcttttggtcctactgggcaataaaaagctatgattctaagcctgttcccttttctcacctaagaatacaaatttgatacaaagaggccgcagaatgtgtcaaacactccctgttgcctggaattctctcttcctttgggttcagggataaaggtatgttatttcttaagtctccctttgctttcttctgcttgcctcgtaaatatttttccatcttggcagtcctacatgtcttctcactctacatgttttccctaggtgatgtgacccagcctgtggcttccactgccatccacacacgtcgctgcctctctccacatcagcatcgcaactatctcctggaagctttccaagtgctgaactacagtaacctcaaccgaactgctgttcattcaccccacaggcttgcccctcctctgcatctttgtgagaacctgagagtcatcctaaactcctccttccacctcactccccacatcaaatcgattaccaacttgtgctgattttatcttcaaatactctccagaattgtcgctgtcatggactgaatatttgtgttcccccaaattcatatgtcctaatccctgatgtgactgtatttagagacgtgacctctaaggagtaattaaggttcagtgaggtcaaaggtggagccctgatctgataggatcagtgtccttataagaagagactagagctgggcacaggggctcacacctgtaatcccagtattttgggaggctgaggtgggaagatcactcaaggagaggagtctgagaccagcctgggcaacagagtgagactccatctctacaagaaaataaaatagtcagacacagtggtacacacctgtggtcccagctcctcaggaggctgaggcaggaggatggcttgagcccaggaatttgaggctgcagcaagctatgatcacacctctgcactccagcctgggtgacagcatgagacccagtctctttaaaaaaaaaaaaaaaaaaggccatatatagcccagaagagcgtcctcaccaaaacccaatcctgatagcacctggaggacttccagcctccagagctgtgagaaaatttctgttgcttgcaccgcccagtctgtggtattttgctgtggcagcccaagctgactcatcagtgaccttctctctgttaccgcagagtagctcatcatcctctcttccctagagtccagccactctctcacatctacctacctagcagtatcactgtgggttagagtcagatcactgcggattaagtcctcattctgccactgcctgtgtaaatctgagcaagttacttaatctctctgtgtgtcagtaacctccctgtgaaatgaggctaataatagcagggttgtttcaacaaggcgatacatgcataatgcttacaacacagcttggcacattataagcattcaacgaaaagtgagctactattatctcatccgttatcagaataaaccacctaagccacaaggctgcccacatcatcctcatgttttaaaacacttcagtgggctccccaccatcaacaggataaagtccaagcttccttagcatttcttagaggctccatatgaatccccaagttccactacaggaacacaggtgaactttccactccaacctcaggctccttcgtgtcactcctcatccacatggaggtaagcagcaagagactccgtgcagttcctggtggttccctgaccctcaggcagactctccccagccctctgcctgcaacgtccttgccctttgcttcccttggccagctcccattcattctccttgattctgcttggaagtttccctctcaggaaggctttatgaaccttagtgtaggttatgaacccatctttgctcctttcataccttttgcaagcctttatttattatgacacttaaccattatcatactgaagtgacctgttggtgtgtctttgttccccactagacagaaaactcaagatcagagaccagttcttgttctttttttttttttttttttttttttttgtatcacagtgtttagcagcctgctatatggtaaatgtcagtaaatgttccacaaactgaatggaattgagctctggaatctagaccatcttttccatacccatcactcctgtcttagttgaagtccttatttcccatttgaagcaatgcaaaggatttcctaactctaatctctcttttcttcacaccatcctttaaacagccgacagaatggtcatcctaaagcacatatatcctatcttacatatcctagattcggaacctctctgggcttctcaccatataagaagaaagtctaacctccttagcaaggtgcataggtcttcaatgggctccacctcacttctctatatatacctatactcttgctacactaaacttctttcttactgttgctggaacaagttcaacgctttcaaacctccctgactttgcatatgcagttcattctgtcaggaatgcccttctctcttatgcctgggatattctcattcattccatatgacctatttcataagtcactccttaatgaagcctttcttagatatccactggggcaatcagctgcttgctcctgtttccacagcacattgttcacacagatagcacaggacttaccacaagttattataattttgtctgtcttgcccatttgaatccaagggcaaggacggaatcattctcatctttgtatgtcctgggaactagaactgtacctgagacataataaacacttgatatgtttgtaatttttaaataagttaatgaacggaatggctagaaaaagtgagaagaaactctggcttactgtatatcatactgtcatactaaaaatatatactgaagacagaatcacattatatcatcacttttcacgctataggccatgatccattatgaaaaagaggatagtaaaaaaatcacagggcacaatttttgtttctgtcacacacatgtgtacctgtatattggactggaatgtaaaacgcatgttccattgtagaacgtggttttaaaagaggcttggaaaacactgcatatggtcatttcttagtttagtacaatttattattttcgtaataacctcagctataatataagtctaccatgaagcattttggggagattaaatgagatgtgaaaagtaaatgtgttagatagactgaattcatatcatagcttgctctgatactttacaaaacatttaaccttacccacaagttttagtttcctcactaaagtcaccctgaggacagtaatgggatcttcctcacagagtattgtgaggaatacataagagaacgtacgtaaatgcctggcacttagtatttattcaataaatcttagcaatgatgatgataacaacatggtacctggcacataagagagttaaaaattagtttcttcagtcaaatgtgcttacattgatagttgatactaactggggttaaaaggtcattgctggcatctcagaaagatagattacagtgaaataaaaaatgactactgcttaaaatgaatgaagacttatttacaaagtcatgttcatctggtacaataatgaagtcgctcaattgggagaaaatgacaaataatacaagtgaatatacaatcttacttaagacgaaagaaataggacaccaggctaactatcagtctcctaaaccacaactttatttctgatacaaagagacagtgagacaatcagggcttccctcaaataaattacttaatctctcttcaattcagttttgcatctgtaaatataaataactacaatttcacagtatttccatttaaaaagttctagtgcaacatcagaaacaagaacttagtaggtgttcaaaaagaaatataagttctgctttgttagccagcaaatagttgcctgtttctagccctcacttcttttctcctaaatccctatattgcatttatttaacttaaagtgctggatgtggcactacgagaaagaaaaagatatttggtaatcttgttaaaatcattagacatcccaggctatctggaatcaccttgggctcacagttagacatcagctatggcttgttttatttaaaaattcatccactgatgcatgataatggaattcacaggagagcaatttaccaaaaaaaagaaatttattgatttataatgtgagatattaatttagccacaaatatttattgagcatctcctacatgccagggaatggactatatatggcaggaaaacagataccaatcatttatatcaggcatttttttctaatagaaggatattcgcaggagacaatgcatagcaccatgccttgcacgtaacagacatttaataactattagttgaataaaattggagactagaatgatacataaagaggcaagaaagagcaaagataagcctttctgagaatttctatcatgttttgctcaatagcttgtctttatccactgcttgtatttttccatgtagctaatcctcattggtcgttagaattgagacaccctttccttgaaatcaggagctataggaggccattcttcctactgggcattttctttctgggacagggtctcactctgtcacctaggctggagtgcatcatagctcactataaccttgaagtcctgggctcaaggaatcctcttgccaaagaggtgggattacaggcatgagtcaccatgccagcctatttggcatttctactgtagacaaagcagacttacagcagtaggtctacctgcctaatacaaaaagaaaaaaaagaattttaacaaacaaatgagggaatcagatccagaaagtgattcttataacttagattacttagagtagatctataatctgctctagatccactgcatacagtgggcccttcttatcatattccataaatagcacttttctcagcccagcttttgatgatagctgaacagactaacagtttgtctaacaaaggctagagaaggggatagcaaataatggcccacaggctgaatcctgcctgctgctcatttttgcaaagttttattagaatacggtcatttccactcattttcacactgtcaatggctgcttttgcgctacagcagcagagctgggtggttggggcaggggtcacatggctaacaaagactaaaatacttatcatctgaccttttacagaaagtttgctgatccttggagtgtacaagtattctatattgttgattaagaacagaaccacaagtattagaagttagaccagcaggtggtaaagctgatcatctactaatataatggaaattggggttcccaatcaggactcttgctttgatagaaggccatcttaacgaggagggagacacctgcaggcaaagtcagaattttctgcaggaaaagttttgagtccatttccccttgtgaacaagtgctcagctatgcatttcatctttagtaaccatgcttctatacctggttctccttggcaaagatttctttcttcagtaagtctcaagactttctgggaaggtagggagatatgggggtaaaagtgtcccaggacttactgaaggaagtgttttatgattatctgatagaatcactgtatcatggtagagaaggcaaacagaatataatctgaaaatagaggtgagggtgaacaaatgggcactaaaagtgaactcagcatcaggaaggtagcaaaacaagacatcagtcaaagatatggggtgattcagacctaaggaagatttaatgtgggatgtttccgtgtgccaggagctggacacttaagcaagaggagatccaggaatgttgctaaaaccatggcctccatactttattggaattagcacaacttatccttgtttctttcattttgcaatcaaaatctttaaaaacacattatttaaaaatacattattttaaaagctagaatgaaaattatgatatcatttaggtggtttaaaaaacatccaccagccgggcgtggtggctcatgcctgtaatcccagcactttgggagtccgaggcgggcagatcacgaggtcaggagattgagaccatcctggctgacacggtgaaaccccgtctccactaaaaatacaaaaaattaaccgggcgtggtggcgggtgcctgtggtcccagctactcgggaggctgaggccggagaatggcatgaacccgggaggtggaggttgcagtgagctgagatcgtgccactgcactccagcctgggtgacagagcaagactccatctaaaaaaaaaaaacaaaaaccatccaccaaaatgggaagaagtgatgaaaaattacagtccaagaagaagggccatagctgtttaaatcaattggtatatttgttatctaatataaccccacgtaacgacaggtatttaacaaatgtttctgctgaatttgacgattccatttcccttacatcccatatgcaatccatcagcaccccacatccaacccatcagtacatcctgtcagcattggctcccaaatataacctaaatctaacacatatcctactatctctgctgctacaactttagtctgaaatctcataatctcccacttgtactactgtagatgactctgaatgagtcttcttgcttccattccacacagcatccatactgatctattttttttttcaattttttgtagagacggggtcttgccatgttgcccaggctggtcttgaactcctggcttcaagggatcctcccacctcaacctcccaaagtgataggatttcaagtatgagccactgtgcctaaccctgactgatctttctaagcataaatctaataatgccccttccttgattaaacccttcaatgaattcacattaagcaaacaacctggccaggtgtgatggttcatgcctgtaatctcagcactttgggagaccaagatgggaggatcacttgaggccaggagctcaacatcagcttagacaacatggtgaaactacatctctacaaaaaatacaagaattagctgggcatggtggtgcacctatagtcccagctactcgggcggctgagctgggaggatcacttgagccctggaggtcaaggcagcagtgagctgtgattatgccactacacttcagcctggatgaagtgagacctggtctccaaaaaaaaaaaaaaaaaaaaaaagaagcagggcaaggtggctcacacctgtaatcccatcactttgggaggccaaggcaggcctcctggatcatgaggtcaagagatcgagaccatcctggccaacatggtgaaaccccatctctactaaaaatacaaaaattagctgggcatggtggcatgcacctgtagtctcaggtacttgggaggctgaggcaggagaattgcttgaacccgggaggcgaaggttgcagtgagccaagattgcctggtgacagagcgagcgagactctgtctcaaaaaaaaaaaaaaaaagaaagaaagaaagaaagaaagaaagaagaaatccttagtcctgtcttaactacttgagaggctgagggaggaggatcacttgaacctaggaatttgaggctccagtgagctatgacagcaccacggtgctctggtctggagagagtgagaccttgtctctaaagaagagaaaagaaaagaatgaatgaatgaacaaaaagaaagaaggaaaggaaaagaagagagagagagagagaggaagaaaggaaggaaggaaacaaaataaaataaaataataaataaataaacccaaatccaacttctttaccctaatcaacaaggctcaaataatctcatgccaactaagtctctgaacagctccttccattctattgccagattactccatctttcagccacaagacctttttatcttccttttaccagccaaacacaatcctacctcagaacatgtgcactttttcttttctctgacttgaatctcctccacccattatataatcttagctcaaagaggcttttcttgacaacttagcgaaagtatttatcccagtcattctctgctacattattccaatttattttctccatagtacatttcagcacataaagatttccttagtatgtgcttgttgcctttccccaacctcctaaaatgtcagcattccttgagggcagagactgtttcattcctgtatcatcagcacctaagacagttcctggaacataccaagtacttaataaaaatttgtttattgactagctatgacacattttacttatataatttcattttctcagcaaaatgaacactttgaaatgtaattaattactgatttttgcagtattttctaattatttaaataaaatatttactattttggtcaaccagaattcttacattgttttagcacccagatagcttctaaaaatgcttacaattaacacaattttatctagcaatatgtatttatcactagacagaatgcactgaactcttcttcattaataaaaagcaatccaggctgggtgcagtggttcacgcctgtaatcctagcatagtggaaggccgaggagggaggatcacttgataccaggaattcgagaccagcctggccaacatggcaaaaccccatctctataaaaaacacaaaaattagctgggtataatagcagacatctatagtcccagctactcaggaggctgagaggtgggaggactgcttgaccccaggagattgaggttgcagtgagccgtgattgtgtcactgcactccagcctgggctacagaatgatacctcatctaaaaaaaaaaaaaaattagccaggcatggtggcatgcacctgtagtcccagctactcaggaggctaaggtgggagggtcacctgagcctggaaggtagagactgcagtgagccctgggtagcccgcgccactgcactccagccctgagtgacagagacccagtttcaaaaaaacacaaaaaacagaaaacaaaacaaacaaacaaaaaaacccaatgcattgctgaaatgttaaatccattataaagaaaagtacaggggtgggcatggtggttcatgcttgtaatcccagcactttgggaggccaaggtgggcagatcacttaaggtcaggaattcaagaacagcctggctaacacagtgaaaaatgcaaaatacaaaataagccgggagtggtggcgcatgcctgtaatcccagctactcgggaggctgaggggggagaatcgcttgaacctgggaggtggaggttgcagtcagccaagatcgaactccagcctgggtaacagagactccatctcaaaaaaaaaaagtaaaaagtatatagttgattctgcagggacttaaaaaagtataaatatcttttttaacatcacaaagctctgatatctgcaggtttatgactaactactagctcactcccatgaatacacgtatgtaaacaggctctatacaatctacaatcccagactaaggggaaaaaactgtcctgtcactgtggtctccaacccttggcccatttctttcctcttgaccacaaaacttctcaggagttgcttgtttcctcttgatccacttatctttagcccactccaatctggcatcggttctcagtactctccactaaaactgcttttatgaaggccatcaatgacgttcatgctgccaaatccagcagacacctcctgttttctaattttttttattgttattttttaagagactgggtcttgctctgtcacccaggctggaatgcagtgatgccatcatagctcactgcagccttaacctccctgagttcaagagatccttctacctcagctgggactacaggcatgcacagctatgcctggctaattactcaatctttaacatagctgataattccctccttgaaacactctcaacttttaagaaaccctgttattttcctcctacatttttagccagttcttctatcagcttctccttatctgacctctaaatgttaagaacattaacaaagactgaacctagtttttttctccccttactgtactgctcctgggcgatgtcaatcagtcccattgctttagatactatctgttgaaacactgaaatcactggttttttttgttttttttttttttttttttttttttgagatggagtttcgctctgttgcccaggctggagtgcagtggtgcaatctcggctcactgcaagttccacctcctgggctcaagcaattttcctgcctcagtctcccgagtactgggattacaggtgtgtgccaccatacccagctaatttttctattttagtagagatggggtttcaccatgtgtccaggctggtcttaaactcctgacctcaggtgatctgcccaccttggcctcccaaaggttgggaaaagatatcccaatctttttcctatgatttcttaattgatctacttgacatatccacttggacttttaataggcatctcaaacttaatgtgttcaaaataaacctcgtgactttccctcccaaacctgtccctacctccctcaataactaatattatcattcttatattcatatattgaataaatgtttgttcccccaagtatttgttgctataaatttatgaagaattcttttctcactagttattataattaaaatgtaatatttattttctttaaaaactttactttgtaggattattattttttaaacagggaccaacaataaataacttctctacttgattaaaactagggcttcctcttgtgctccctcaggactatttctttgtaaaaacaataggctaaatcagtactggtgtcaaagaaatcataatctcacaactttataaatacagcatgtggcaagggattttcccatcttatatagtaataaaattttcagctgtgccatggctaaaagtttaccatcaaagttggaattttaaattagaggtagtcatctttctttctttttaaagaaatggagtctcactatgttgcccaggctggagtgcagtggctatttgcaggcatgaccacagcacgctacagcatcctggcctcaagcaattctcctgcctcagcttgccaagtagctgggactacaggtccctgccaccacacccagcagaaatatttagctttctgaatttctcaagtgtgtgtatgaatgagactagtggggtccttaaccaagattcacaggatttttagtgatttattaaataacttggatttgtatctaccagcatgttctttgaggtacaggtatgtcttttatatctcctaatatagttcattacaatgctaaatactaagatgtgatgctcacacactacagaatagccaagcaaatgaactacttattctcatagggctattataattaacaaattcttgtatcaccccatcattatcaacaacaacatgataggatttccttttatcttgaagagtctggaaaaagggtaacagagagatatttctgaggaacaaactggtaatgagggagctactgtgtccattacaatactccttctagaagctcaatacataatgactaatctctggaaaaaagcaagtgtgagaatggaaggctcttcttcaaactatgcaaaatgaatcaatcagcagtgaacaaatttatgagccaaacaaattcctacaaaaattaccatcatatgctgtcatgcatgtctgccagtctatttatcatattatttaagaaacaaacatttattgaagatttatcatgtgctcagcactgccaaagaggaaataaagagcataatatctattcttagaaaataacattaacacaaatagaaaacaagaaaccataatgttaaaaatattacatagtaacacagaaagacaatgtataattatacatacgcactaaagcaaagataacataatttataaattatgaggtacagaatagttagattctgaaaattaaaataatcaggaaaaacttcatgaagatgagatctgggctggatcccaaaggataggcaggtggatcatgtagaacaggggaaaggagttcctgatcggggatacaatatatgtaaaaactcggagacaggactgagcgtgaaatgttaatgggacagtaaagaaatcttcctctgcagcgggggaaaaaacagaataatgggaaactgcatggttaaaaggtttgatgttaagatagtgcttggacacaaaagatcttaaagttgagtcaaaagagtacaatgaaagcattagaaatagaagataaaacacaattaggccgggtgcagcggctcatgcctgtaatcccagcactttgggaggccaaggtgggtagatcacttgaggtcaagagtttgagaccagcctggccaacatggtgaaaccccgtctctactaaaaatacagaaattagccgtgaatgatggctcgtgcctgtagtcccagctatttgggaggctgaggcaggagactcgcttgaatctgggaggcggaggttgcagtgagccgacatcgcgccactgcactccagcctgggtgacagagcaagcctctgtttaaaaaaaaacggtaaaaataaataacatttactattgttttctgatgatatatatggcctctaattgtaaagctgaatgcctagtttaccactttttttttttttttgagacggagtcttgctcttgttgcccaggctggagggcaatggcacgatcttggctcaccacaacctctgtctcccaggtttaagcgattctccagcctcagcctcccgagtagctgggattacaggcatgtgccatcatgctcagctaattttgtatttttagtagagatggggtttctccatgttggtcaggctggtctcaaactcccaacctcaggtgatccacccgcctcagcctcccaaagggctgggattacaggcgtgaaccaccgcgcccggcctatcattcttattttatgcattaggaaactaaggctcaacaagattaaagctgtctagggtcacaaagattgtaagtggaggggctagaattcaaaatgagacctgcttgactcctaagcctgtaccatttctactatatttagagtgaagtagatgggttgaagaaatatttaggaggtgaaatttcaaaagtgtacagtcagaagagaagacatatatggaaacctaaattttcacacagtaaagtgtcaataataaaggcataatgccaaaatgacagaggctgtgcatggtggctcatgcctgtaatcccagcactctgggaggctgaggcaggaagatcacttgagcccaggagtttgacaccaacctggccaacacagcgaaaccccatctctactaaaaatacaaaaaattagctggtaatggtggtacacacctgtaatcccagctactcaggaggctgaggcattagagtcacttgaacctgggaggcagaggttgccatgagccaagattgtgccactgcactctagcctgggcaacagagtgagactctgtctcaaaaaaaaaaaaaggaagactcgagggctagaaccctgaaattgggaatgaacaggactggctgaaaatgtttcttgcacctgataaaaatcttgaagaagaatgctttaaatagataagaaaggagagagagaggtgggcagtgagaggagaccaccctaagtaatcagagattacttacgttggttactcaggctggtctctgaatctgattataaatgaaatagagattacttaaaacaaagggctgtaaggtagcactgtccagcagcactttctatgatggaaatcttctatatctgcactgtccaataaggtgtagctgctagcacatgtggccactgagtacttagaatatagctacgacaaccgagaggctgaattttaaatttaatttaatgaattcaaacaaatttatttttaatacagcactttaaattttatttttaaattttaatctattatttatttagagactgggttatgagactggctaatttttgtatttttggtagagacggcgtttcaccatgttgcccaagttagtctcaaactcccgggctcaagtgatccacctgccttggcctccccgcaaagtgctgagaatacaggtgtgagtcaccacgcccggcctaaacttaaatttaaatagccacgtgcgggtagtggctaccatactgcacatgcaactgtaagatgtagaagtcagatgtgagcaaagaaatgacaagccgttcaatgctgttagagaatgaaattcaaggttccaatgatctgaacttgtgtcccctcaaattcgtatgttgaaatcttaatcctcaatgcaacagtattaagaatttggggctttaggaggtaatttggttttgagggtggagccctcatgaataggatgagcacctgaggtagcctctttgacccttccaccatgtgaggacacaccacgaaggcaccatgttggaagcagagagtgagcactcccaagacactgaatctgccacatcttgattttgggcttctcagcctacagaactgtgagcaataaatatctgctgtttataaattatccagtgtaaagtattttgttatagcagcctgaatagactaagacaaaggtggactaaggcaggataacaggttagaaaaggaggcagggcctttttttttttttttttttttttgagacaaagcctcactctcacccaggctggagtgcaatggcatgatcttggctcactgcaacctccacctccagggttcaagcaattctcctgtctcagcctcccaagtagctgggattacaggtgtgcaccatcacacccagctaatcttttgtatttttagtagagacggggtttcactatgttggccaggctagtcttgaactcttgaccttaaatgatccacccgcctcggcctcccaaagtgctgggattacaggtgtgaaccatcgcgcctggccgaggcacagtgtttttacagagaagcctgtttaaggtttaatcatataaaatgtatgatatccagtaagttttgatataaaaaagaaacacctggcgattttatataatatattgtgctaaggaattttaagcactctacattctgctctctaagctctgtaaagagcaccagggattttttttttttttttctttttgaacagggtcttgctctgtcagccaggctggagtgcagtggcacaatcttggctcactgcaacctctgcctctcgggctcagcgattctcccacctcagcctcctgagtggttgggaccacaggcgcatgccactacatctggctaattttttgtagagatggggttttgccatgttgcccaggctggtctttaactcctgggctcaagcgatcctcccaccttggcctaccacgcatgcctggccacaacagggatttttaaatgtaagactacctagtcaactcttattctatattaacaatatagacaagaaataacctctaagtaatctctatttcatttataatcagattcagaggttctcttatgctttacaatattgtcctactgtgggtagcgcaataactaaggtaatctgaaagaccagttatattatatactatagttaaatgcatttcaactgcatgggagaaagcaactgtgttctttcctctcaattttaacagaaggaaaattgtcaaaattagcttatttagaatgtcctatcagagaattattttgattaaaatatatttttaatcaataaaatatttctctttggtcaatacttgtcaatatagaataatatctagccacaaaattaaaaaaaaaacattttcccctatattacattcatggatcttcttgaatttctgttatctaggtgcttttaaaagtcatatttctgataatatgaaatcacagctccttttctttggcatatttagttactgtattaagaaaatgtacaacacataatttagaatgggtaattattatattctctttattcttatattgaaaatgacatgaaaattaccagtcttcccaggtaatataatttaagttaaagaacatctacatactacaaccaatacccattcccctatgttatgtttggaaaaacatagaagtatctttagtagtactcttagaaattatcccaggttcagcatattggtattttatttccaggtttaagttacagtattttgggcaccccaagtttaataaactattccctgcagaaacctgacaagtgaagttgtggctgggaatatgttagtcttcagataaaatgaattgtttaagaatttgctaaagatctcaaagcatctttcttaaatctaaagaaagtcaggaacaaagccacaaccaggaccatagcatcagaagatggaaagttgctttgtcttcaaacttaaaaaacattttccattttaaaataattttactatttacctgtgatactgttgaaaattatgaaaaaacagataatttaaaatttagtgcttttttttaaaaaaaaaaaaaaagcgaatccctgggacacttcatatagtgcaaaacaacaattcaagaattcaagcattgaaagaaataatctcttatcccccagtctctgaaagggattgcctttactactgttcccatctttatgtccatatgtacctaaggcttatctcccacttacaagtgagaaactattcagtatggcttagtcatttttaatgcaagagaataggtaaaaatgccaagcaccagccagagttttttctttgcagatagatgtgactcttacaggagcagcagggatttcccactttgggcggaaagcagcatttaggtattccccctccagtgcagttacagaccacccccccgtagaagctgctcctgtcctctgtggcatgtcagcctctgattatcttttaataaacaatatggcatattaagtctcttttatgcccttctttgtattcccaggtaccacctccatgtcaggataacaagaatttggtaatgtttgttgaataaatttagcagaagttgaaagaaaaatcctgtttctacagaaagataccactggcttttggggagcccgagttcatgatgaaactaaagaaagccacaaaagttcacctcaatgccaagacatttcttgatttttgaaaacccagttgtcgaaccacccatctatagaaacttgaaagactaaaaactatcttactctaaacattttctaggaagttgattctacaacacattttggttttccaatttggcttctaataattatttcaaagtttctgtggcctaaattttgttttacattgatcctttgaatggactactgtttccacattttagaacatttaaaaagatatctacaacccgagtctaatcataaaaaaaatcagacagatccaaaatgtggaacattccactaaaaaaggagtggggagaggtctttattcttccaaaaatatcaatgccataaaagacaaagacggctatggaaatgttacagattgaaggagactaaagttaaatgcaagaaaggaaaaaatggcatataggacagtattgaattgactgacaaaactggattacaatagtagagtatcaatgttaaacttgctgaagttgctaactgtatttcttaggaattattcacctaagaatttaggcacacagatatgatgtatgtaagttacccttaaatggcttagaaaaaaatgtgtgtatattcatttacatacgtatctacacacacgtgtattagcggaagagagcaaggcacacatgtgcataagtgataaagcaaatgagatgaaatctttatttttaaatttaattttgtaagtttcagctttttaaaattttagattccggggatacacgtgcagttattacttgggtatattgtgtgaagctgaggtttggacctctaatgttcctgttgccacaacagtgaacacagtacccagcacgcagtttttcagcccttgccccctccctcccgctctccctccttgcttttggagttcccagtgtctactgttcccatctttatgtccatgtgtacccaagacttatctcccacttacaagtgagagcatgcagtatttagttttcttgttctgcgttagttccgttaggataattgcctccagttacattcatgtcactgcaaaggatttgatttcattctttttaatggctgtgtagtattccatgttgtataggtaacacattttctttatccactcatcaattaatgggcacttacattgatttcatgtgtttgctattgtgaacggtgctgcaatgaacatctgagcgcaggtgtctttctggcagaatgatttattttcctgtgggtatatacccagtaatgggattgctagctcagataagtatttctatttttagttgctctccacaggggtagaactaatttgcattcccaccaacggcgtgtaagtgttcccttttctccacggcctcgccaacatacgttcttttctgatttttaatagtagccattttgaactggtaagagatggtgtctcattgtagtttggctttgcatccaaatgagacaaaatcttaatgacaggtgaatctaggtaaaaggcatacagacgttctttgtgttgtttttttaacttacatttgaagttattttcaaatgaaaaataaaagcaagcaaaaaaaggtcattcttcatctagtaaactcttcaaagattaccacccccttcaacagtttttcctggttctagtgagtcttctcccatttgtttagatctttgttgaaatgtagtctcagataaaaaattgtatttttatttcttttacatatttcaaacaatctaaattctttttaaatgaaactcattaaaaatactgcatttgtttctaaataaaatggtagaggtaatttgcacctttccaaacagaagcaataggagcaacccagatgttctagccacgatccaagtcaaccacattcaatctaagaagtaattgaaggctgtaacgacttctgtaaggcctacaaaaatgagttcagacacaagctctgctcagtaaaaatctagtggcagatgatatatacaatgatctgagaaaaaggcagaatcaacaaaggttgtatttttatctattgctgcgtagcatatttccttaactttagtagcttgaaacaataaacatttattatttcataaagtttctgtggtcagaaatccaggagcagcttaactgggtggatctggctcagctgtagacaagatgtcggctgggacggccatcctttgagggctctgagggctttgagggctgcacgatccaattgcaaggtggctcactcacatactaggcaagttactgctgggtgctgggaggagaccttagtttcttatcacatggacctctccacagggctgctggaatgtcctcatgaccttccccatagtgagtattccaagacaggaaagtggaagccacaatgtctttcatgacctagcctcaaaagtgacatactgtcatttacacaatattctactggctgtacaagttaatcctatttagtctgggaggggactgcataagggcatgagtaacaagaggcaagaatccttgggggccatcttggaagctggctacacagaagagaaaacaccaggggagtgcgaagaaggtgcaattaaactcaattccttggtatgccaatggtaagaaatattaggtgatctctggggtgtaacctttttaatttagttcttcactgaataatctggccagtaattgtaatacaaaatacggcactctgacaatattctctccctttataatcaattacacaccagaatatatataaagaaagacttacaaagtcacaagtaattgtttggtattatttttataatcacatactagggccctacaattagcattcacaaacatcactccatgttggccagataagtctgtctttatagtggtttaccatacgcgccttagcatgaagttacatgtggtttccttagccatcagatgctccaaatgcaaaaaatgtctcaccacagtcacagaatcatggaatcctaaagttacctggggtttctgaaaatctcatgggaacaactcacgagaattaaggcttaagaaagtgatttatcaaagaacaaaaccagcaagacttgagtttagaactcgcagcagagttgtgactagaacctgttgaaataggcaatgtagaaacccagactaaggcacattctctacaactttactatgcaagtatgcttagatactccttagcaaacagcaggccttgagtaaattctttcagaactgaatacacaaaggatacagaacggaatacactaacaatagtgcatgatgtgctcatttctgtaatagaaatgaattaattctgatccatctataatttattattgctccatgattaacggaaggcataggaaagatgactggaatagtgtaactagtacaaacaagtattacacttgactgaacctcattacactgcaattgcatattatatagtatgtaggtgaacaaatactgggttagtcagtggacctacatttgaatactggttctgctcctagacagctgtatgatttgaatgacttctttatactttcatagtttctctgttcttctctgtaaaacaaaggcttagaagatattatgggttagattatgccccttacaaaagatgctgaagtcctaaactacaatacctgtgaatgtgactttatttggaaatagggtctttgcaagtgataaagaagaggtcatggagtgacctaatccaatacgaccagtgtccttataaaaaaaaggaaatttggatacagatacacacaaacaaggagaatatcaaatgaacatgaaggcagagaccggggcggtacatctacaagccaagggacaccaaagattttcagcaaatcaccagaagttaggaagagtcatgggacaggttctcacagtcctcagaagaaacccaccatgtcaatacatcattttggacttctagtcttcagaaccgtaagaaaataaatttttgttgttcaagctacccaatttgtggtactttgttacagcagtcctagcaaactaatacaaatgagctcttaacactggtctaaaataggataatcctatgaaatgctacaaatgtttgggaagatttctcatactcaactgtttacagtataccacaagcctgtcagttgaagatacaaacagaccctctataatcctctatacttatatgcaaggaacagcacactttttctgcaaaaggtcagatagtaaacattttaggctttgtgggccaaacaaggtttctgttacattttttttttataactccttaaaaatgtaaaaatcaccctcatcccaacggactacaggaacagacctcaggtcacatttgactcatagcctgacccctggtgtgtagggttaacaagcctcctttccctgggctcctttttctttcagcattccaagccaaaggaaactatctttttcaaatcattttctctcctaggtgggacatcttacaccagcccaggcatgcttccgatagccttagagtagctgtcccttcctcagaattactgtctaattggctagaagttagcaactttttacatttttccttcaattcctttccattaagaagaaggcatgcaccggcaaattacttgtgactatcaatgacatactctcagaagcaccagtacccctgtgttgtttctaaacccattctaatagacacataccccaaggttatgctgtttgtcatctcacaaaatgacttacatctagagatttaaataattaatgtacttttcataactaccaggtacagtagatctgataatggcagagctaagcacatatacagaaagtagggcaagggccagagactcattttaaagcaatgttacaagatcgtcactgttgcttttcatttttctaaatgtggccactgctgttttctcactaaaggaaatgttttatgtaaagtgaataacagtacctggcataaaataagtgctcaataaatgttaaggccttctctccctcttcaactggcctcctcatttttcacaaagtgaaatagaaaaacaacatggaagataatcctgttgcttaggaaaaataactaaagcttgctagacaaaatacacctgaaaatataggaagtgagctatagctggcctatatgcatgtatgttggaacaggacaagatagtgtagggtggggtgaagaggacagagaaatggaaggaaaggggctacagccttggtggcaaaataaaggataagacgactcttttaaaatggtctatttcaaatgctgggttgtgaaacttaatttgattacttcatgagaaacagcatctataatccatccctgatttttctacaacaaaaatttattatttattttatgtttgtgtgtagatcttttatatatatacatgtacacacgtatatgtatatattatatatgcatatgcatatatatgtgtatatacatatataatatattgtgtgtgtatgtgtgtgtatatataatttttttaaaggaatggggtctcactatgttgcccaggctggacttgaactcctgggctcaagcaatcctccacctcagcctcccaagtagcaaccaacagttttagttttgaaaaaataacaaatattaaacacccatgtgtaagggttggtactgggccctgtgttagtttgcatgggctgtcgtaacgtaacactacaggccgggcacaacggctcacgcctgtaatcccagtactttatgaggccaaggtgggcggatcacctgaggtcaggagtttgagaccagtctgaccaacatggagaaaccccgtctctactaaaaatacaaaattagccatgtgtggtggctcatgcctgtaatcccagctacttgggagactgaggcaggagaatcgcttgaacctgggaggcggaggttgtgatgagctgagatcaggccattgtactccagcctgggcaacaagagcaaaactctgtctcaaaaacaaaaaaacaaaaacaaaaaaaccctgataacactacagactgggtagctggaccaacagaaatttattttctcacagttctggaggctggaaatctaagataaagttgttggctggtttggtttctgaggcctctctccttaacttgcagatggctgctttcttgaaatgtcctcacatagctgtccctctgtctgtttctggtgtctccccacgtatccaaatttcctcttcttataaagatactagtcatattggattagggtccaccataaagacctcatttaaacttaatcacctttttacggccctgtgtccaaatacagtcacattccgagttccaggggattagggcttcaacctatgaattgggggtggggcacaattcagcccgtaacaggcctagaccttaatttgtcaacactacagttagatttatagtatagtaactgcatctgtgctcatctaaatgtcatacccaaatgaaataatatagcatgatgatctgaatttattaaaggcaatttttcctatagaaacccaaatctataaattatatacaaactgtggtaagttactcgataccttgccaggactcatctatggtggtagatagaccacaaagagtaccactgaaagatccctttcctaatcacagtttcctcactggcttgccacaaaacctaaaattcttctattctttcattggcaatttatttcccctgaaaatgtaaataatctctggcagagcaatctattaagtgatcatcagccactaacaccttagggtagaacagctcagatcacagtcttaaaataaattccatcagtatgaaattttctttattactgctccgctactggaatgttagatcactgtctgctttaataataattctggtgtaggtcattcaaattttgtttaagataataagacaaatagcaggtataaaaacattccgtcatctaataaagcaacccgagaacagtaagaagaacgtgatgaaattaacatttttgagtacctgctaggaatcaagtattctgctagatattttagaaatcatctcaattcaatcctaaaaattattctgtataatagtataggttgagtattcctaatccaaaaatctgaagctttttttttcctgagacggagttttgctcttgttgaccaggctggagtgcaatggcgcaatcctgactcactgcaacctccgcctcctgggttcaagtgattagggatactcaactggctaaatataatgcaaatatttcaaaatctgaaaaaacccaaatctgaaacacttctggtcccaaacatttcaggcaagggacactcaagttgtattaatcccattttacagaagaagaaacaggctcagataaatgaacatctcagagcttgttgatagcaaaggagagattgaaactgtcaggcctctgatcccaagccaagccatcacttcccctgtgacttgcatgtatacatccagatggcctgaagtaactgaagatccacaaaagaagtaaaaataaccttaactaatgacattctaccactgtgatttgtttctgccccaccctcactgatcaatgtactttgtaatctccgccacccttaagaaggttctttataatttcccccacccttaagaaggttctttgtaattctccccacccttgagaatgtaatttgtgagatccaccgctgcccgcaaaacattgctcttaacttcaccacctatcccaaaacctataagaagtaatgataatccaccaccctttgctgactctcttttctgactcagcccgcctgcacccaggtgaaataaatagccatgttgctcacacaaagcctgtttggtgtctcttcacatggacacgcatgaaagaaaccctacctggttctgtgtcttacctgttgggggcctgtggtcaaactactagtacggagttttagtgtcctcactttaaaaatgagggttgtggccgggcgcggtggctcacgcctgtaatcccagcactttgggaggccgaggcgggcggatcacgaggtcaagagatcgagaccatcccggctaaaacggtgaaaccccgtctctactaaaaatacaaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaaaaaaaaaaaaaaaaaaaaaaaaaaaaaaaaaaaaatgagggttgtaaggtaactacctactttttatagcattgtagtgaagttgaaatgaattaatccacatatattatagtgtggtagaatgcagcagaactgatgatgtatgacttctaagactagtccttaagagacctgcagtttttgcttttgccctcttggaacactcctgttgccatgttaagaaaaactctggggagactatgaaggaagagagcatactcggggcaggggggtgaacaggacgtgcacatgtacgagcgtacaagccaggtgacaccagtaccacagcctcagacatgtcaccggggataccagcaccacagcctcagacatgtcaccggggacaccagcaccacagcctcagacatgtcaccggggacaccagcaccacggcctcagacatgtcacccagggacaccagcaccagcaccacagcctcagacatgtcatcggggacaccagccccatggtctcagacatgtccctgaggcccacttagacccttcaaccccagcccagctgctaactgactacagccacatgaacagaaccaggtgagaccagaggaaacttccagtcacctaccagatcatgacaaataataaacgatgttttttaaaccacaaagatttggagcagcatttgttacacaaaattagacaactattacagttcgactaaaaacatgttcatttacaatactaaattagaagtgtaagaatgggagaaaaacttcatactttaaaagtcattttttcctccaaaaacttccaactttgaaaaactgatttttataatgcataaaaattaaaataaccttagaatttatatgagtagcatagccagctggctttattatctgttgtactcaacacttcaataatcactgatgttttagaactcttcagatttagaactcttgcccttgctttagtctggtttaagctaaataattgttcttcctcaagaacaaatgaccttacctcgttttgttttccttgtctgagagaaacacattagcagtctcccatcttgtttttccttttcctgtcacccaggacagagggcagtggtgtgatcacagctctgcagcacgacttccccaggttcaggtgatcctcccacctcagcctcccaaggagctgggaccacaggcacatgccaccacgtccagcttaattttgtatttttttggtagagatcaggttttgccttattgccccaagctgatcttgaattcctgggctgaagcaatctgcctgccctggcctctccaagtgttaggattacaggtataagccaccgtgcagccttatattttgttttaaattttcctctgtatttttctctctggcaaattgtttagggagtttctttagtttatcagactaaatttcaaggctttccttccaattttgacatgtaaacagtccctcatttctgcttatctagtgattattcccaaatctgtgtttacagtctagctgtctctcctgagattaagacttgtttctctaactacctgacggcagaatctcctcttggaagtatcaaggaggcagttcaaaactgaactgggcattggctccactccttctccttctctttactattaataccctttctctccttctatatgaccacactaagtcttatttaggcatcgtttcttctgggagacctttgtagaatctctgaggttatgttaacatgctaaggttttcttgacattctcagattgggttaggtgaacttttagcaacttatctttttactaaaaagtcatccctcagtatctgtggggaattggttctaggactccctaaggatatcaaaatctgcatgagcagcccaggtgagaccagcagaagcactttacagtcacctacaggatcatgacaaataataaatcatgtttaagccacaaagtcctttacataaaatggtatagtatttgcatataacctacacatcttcctgtatcctttaaatcatctctagtttataatacctcatacgatgaaaatactacgtaaatagttgttatactgtattgtttagggaataatgacaaggaaaaaagtccacgcgtgttcagaatagatgcttttttttctcgtctaatattatggatccacagttggttgaatccacagatgtggaatccatggataccaaggaacgactgtatgcattttgacaattatacttctcatcttaccatgcattcaacaaacagaacatgtaaagcggtgataatgctgtgatgaaaaataaagcaggggaagaggctgcatccatctagtggaaacgatgcccttttcaatctgcacaaagagaaaaagctgctctccaagttggggggtgggtgggtcaggtatgtaaattggtcaggaagggatctgtaggcacttacagatttgacgctaatgagatgggaagccacaggaaggttgtgaagaaaagacaagacatgatctgattcatgttttgatctgatacactggttgctagatggagaataagctgcatggcggtgagaggaagcagaaacaataggagggtaatgctataatccagtggtccataatccaatatccccccaaggaacagttcggcaatgtctggtgacatttctggctgtcacaactgttggggcggagtgctacttgcatctagcaggtagaagctagggatgctactaaacatcctacaatgcacaagacagcccttcccccaacattgctggcccaaaacgttgatagtaccaaggctgagaaactctgttataatctgtcctagaatgtagcttggattgagatggcagtggtaagagctggagaagtgcttagcttcccaatgtttttttgtttgtttgtttttgagacggagtctcgctctgtcgcccgggctggagtgcagtggcgtgatctcggctcactgcaagctctgcctcctgggttcacgccattctcccacctcagcctcccgagtagctgggactacgggcgcgtgccaccacacccagctaatttttttgtatttttagtacagacagggtttcaccatgttagccaggatggtctccatctcctgatcccgtgatccacccacctcggcctcccaaagtgctgggattgcaggcgtgagccaccgcgcccggcctgaatgtttttaaagtactggtgaccatattcgctgagggattaaatgtaaggtatgaggggaaaataggaatcagacaccagggtttactgcctgagcaatgagaagaacgacgttcctcatacggagatgaggaagaatgtggaatagcaggtaaatagcatgtgcttgctttgtttggggctgtgcagaagagactgatgggaccaacgtgctcagttctggatatattaaacttggaatgcctatttggcaccaagtgaatgtatcaggtaggcagatggataaatgagtctgaagttcaggggagaggctggggtggcaatatgaacttgggagtctccacatctgaatagtatttaaagctatacaacaggataaggtgatttaggaactaaacacaaattgagacgagatccgagcccagaggcactccgatgtttaaaaaagaggaggaaccatcaaaagatactaaggagaagccaagaagtaggagaactgagagtctgagagaatcattatactcatttgatcgactgcaacaaatgctgcttagaggtcaagcaaaatgaggactaagcaaggaccaccaggtctggcaacatggaggccaatgccgacgtggaaatgagagttttggtgggaagacaggaataaaagtctcacaggtctgaattcaagagagagaacagcagaagaagggtagaggtggtagccataaacaatgatacattctcttgaggccttttcttgcaaagctcagtgaagaaacatggttccagagagggatttttttttctctcattttacatatgcaaacatataaaaaagctgaaagaattgtttgacaaccacccttattcttaccacagattcaacatttaatgccatatgttttccctgtatgtactgtgtattgtttgaggataacttcccctctaaatatacctcggatgtatctcctaaaataagtccattctcctacatagccatagtaaccatgaacacacctaggaaaattaaaaatatattctcaaatatattatatagctgggtatattacaatttccccaatatgtgatttgcaaaccaggatcaagtcaaagtccatgcacagcatttggttgtcatgtgtctttggtctctattaataatgatgactgtttgaaaagacctgtcctatagaataaatttgactgattatgtcatgccattgaacttgtttttctattctagaaggatagttttttagggtagtgaatacatttattactcttggcacaatagtctaacatttcccaatttccttatatctctgccctttcattttcagaaaatcaattattccaagatttgtttttcatttatcatcacttattagctctgaagactcaactgagcaactttcagggtttatataccctatattcagaaaaaaactactaccatctctcatttaccctaagaattcataggagagcatgtcttaaagctgatcaataaccaaaccaaacattttattgatcatattacatttggaaagcaaaatgaatttcctaaaatttcttccctgattagcaaaatagtgcctccgaacacttgagggtgaaagttgttgtcaaatatgcctacatgactggaaattatgacatccaaatgagttcactgggtctgataataatatgctctacatgcttatgtctatgtaataaacagcttacatctggatgagaaaattgattatacaaatatttgggcttctacaactggtcactcatctgtaagtacttaaagcaacttaaaatgcaaactgacctaacaatgcttatggttagaattccaaagaatgtttaggcattgtcaggttatgttaaaacatcttctgccacaatcttcaagtgatttatcttttctgttgtgttgaatagctatagaagacaaatgaattctgcactcctgaattcaatgaacatttcaagtttcctcacttacactgtaagattacgtagcatattttaagaaataaattataatcattttatttcacttattgaacttcttttaagctttggcattagaattttaatcaaagcactgccacttgcttacagtgatggtttttaggctctttgggcctatggactatttcaatgaccttcactagccatctagtccaccttatcctaattattaccactgcaaaagaaaccctcacttgaataaatcagtagatgggcatgaggcacctcccaggagactataattattaactcatactaaaatcaaaattgtagctattatcactcatatggtttggctctgtgtctccacccaaatctcatcttgaattgtaatccccacgtgtcaaaggagaagcctggtgcgaaaggactggatcatgggggcggccttcccccttgctgttcttgtgaaagagttctccgatggtttaaacgcatgggacttcctcctacttgctcgctctcttctgccaccatgtaagatgtgccttgcttcccctttgccttctgccatgattttaagtttcctgaggcctccccagccatgcagaaatgtgagtcaattaaacctcttttctttgtaaattacccagtctcaggtagttctttacagcagtgtgaaaatagactaatacaatcaccttatggtaagtctgtctataaatcacctgaactttcacagactatctagaagaacatgtaaccagagtagttcttgatcatgctatataaattactgatacagaaatagagctagacaggaaggggctggtagtagagaatcatcctctggacatattctcacagcctaatctctagctagcaaattttataatatatataaaaatacaattatttcacaaaattaccatgaaacgattttattgggatattagacattactgaattacttgttctgtgaggtatacagtgaaattaacatgttataaaattgtggtagccggcccccaagatggcctccaatgaatccttcacctcttggtattcatacctttgtgtaggtaggtctgtgtaacccatagaatacagcacagtgacagtaggtcacttccgaggttaggttgtgaaagacactgtggtttctgcctctctctcagatcacgtgctctgggggaaaagccaggtgtcattttgtgaagacactcaagcagcctttagatgactgcaaccacataagaggctccgaactggagccactcagctaaaccactcccagattcctgaccatgtatcatttcatacacaatgtatgaaatgacaaatgtctgttgttttaagctgtttggggaataatttgttacataacaaaatataactaatacaataatacatactgatttaactgaagttgtaacttcataacttatttaggtactaaaaatcacagcaacccgatgcaaagtactaaaaaaaaaatccattaatacctattgagtactgttgagggcatgaggaaagctctttcatactccacataaaacttccttaccgtaatattcatggctgacctctactcttaactcctttctaggataggaggggctaactgatctgacagcaagtttgggagaaaaaattctgaggctcggccaacttcctctcttctttccatttgggatttggctgactgaagagggtcatttgttttggcctgctctcttacacagtaaatgtagtgggacaagctctattcttgttgatagaaaaactcgaattttaaatctgcctagttctttgcagctcgttgttgctccaaatctcagctaccttttgaaacaacttttttcagtaaacttaatttcaatcttcatgtgatttaactggatccaaacacaggcagataaaaaaggtggggcattacttatcaacctctaaactaagtttaattttgtgccctcatggagtttatagtatatttgaggtttaaactaaaacacctggttttaaacagaaactataaaaaacacgattaataggtgaggccgggcgcggcggctcacgcctgtaatcccagcacttggggaggccaaggcgggtggatcacgaggtcaggagatcaagaccatcctggctaacacggtgtgaaaccccgtctctactaaaaatacaaaaaattagcccggcgtagtggtgggagcctgtagtcccagctactcaggacgctgaggcaggagaatggcgtgaacccggaaggcggagcttgcagtgagccattgcgccactgcactccagcctgggtgacagagccagactccgtctcaaaaaaacaaacaaacaaaaaacaaataggtgaaaggccgtgatcattggtaagcgtaagaaaatctgagggagaaaaaaatatagatgcccaggccccatgccaaactcatggaatcatgcatgaaacccaagcagctgcagttttaacaagttcccaatatatagttgacccctgaacaatgcaggtttgaactgcctgggtccacttataaaatggatttgatttttttcaataaaagttacaccgagtgtgcctgcctctcctccctccctccctacatgctcctgctcttaagcctctgccatgaggcttaagacagcaagaacaacccgtcctgtttatttcaatagttttggggggtgcaggtggtttttggttacatggataagttctttagtggtgatttctgagattttagtgcaactgtcacctgagcagtgtacactgtatccaacatgtagtcttttaacccccatccaaccttcttccccaacccgaatccccaaagtccactgtatgattcttatgcctctgtgtttttatagcttagctcccacttttaagtgagaacataccatttttggtttcccattcctgagctacttcacttagaatactggcctccagctccatccaaattgctgcaaaagatattatttcgttcctttgtatggatgaatagtattccacgatgtacataaacattttctttatccactcagctcctcttcagtctactcaatgtgaaggtgacaaggacgaagatctttatgatgatccatttccacttaatgattagtaaatatacttacttttccttatgattttcttagtaactttttttctctaacttactttattgtaagaatacagtatataacacatatgacatacaaaatacgttagtcaacaatatatgctatcagtaaacttccagtcatcagtgggctattagcagctacgttttttgggcagtcaaaagcatggggaaggagagggtggtccctaacccctgtgttgctcaagggtcaattgtaataatacccatttaagaatccatggtatatatggtaagtgcaacaactctagaagagagtgctaggagttggaaaaggaaagagaaaacagaatttaaagcaatctgtaaaggacatgcagggtttagatgaggtggaagggtgagggaaaaccaacatctgctgtgagggcatattaactgccagacattgttctatgtcttacctcatttaagagaatttcatttcacacatggaaaaactgaagcccagagaggttaaataatttgcctgaggccaaaattagttaaataacagaagtgggattagtagatgttttcattttatcagtgaaactgagcctcagggaggttaaatattttgtatgaagtaacaaaactgagattaatatatggccaagtttaaatgagatctgtaaatctaatgcctacactaaaacaaaaaaaaaaaagtgggaagaaaaggtctatattgcttagcaaaacagaggtagggaagcaaaaataaacttacaaaatcagattagaccaccaaaaaacagtccccattttaacttatgtggtgagaaccatatattaaagaccaccagtggcttaaaaatctttttaaaaaatgaatctgttttcattattcattagtttttatctaatgaataatgtatcttaactgatacatttactaaacaattaccagctccaattagcactcagttacaattcaatcattaaactgaccctcaatttagctgtcaacctagtcaaaacagttaagtgattttacggtcatcctcagttgcagaagtataatgtttatggctggagtcattttatttttaactaacattttttaaaaagattgctttgtaacaatgtgttatgagtcctttgtggtaaatactgctttttttttgagacgcagtctcgctttattgcccaggctggagtgcagtggtgcgatcttggatctgaggctcctgcctcagcctcctgagtagctgggactacaggcatgcgccaacgtgcccagctaattttttgtttttttagtagagatggggtttcaccatgctggccaggctggtctcgaactcctgacctcgtgatctgcccacctcggccttccaaagtgctgggattacagctattttaaggactttttaaaaagtgaagctaaacatttattcatccctattcctcatctatagggacttgtgctctatttttctttgaagactgaagtaaaaattcacctttgtgagggtcttcctataattaaaattaatcattttttcctccatagcttctacaaaacattgcctgtacaactctatttagcacttatttcatcccgccttgtatgaaaactatttgtttacaaacgtttctacttctctttaggaataaggactatgcattattcactgttgtattctccctgcatttatggcagtcctttgcacattaaatacaagctttttggctctgtgcatctcttcatctggctgttcatctgtaccctttaaaacatcctttattaaaaaaacagtaaatgtaaaaaaaaaaaaaagccattgatgaaaaagttaatagctttctcaataagaaaagagtatcaattatgcatacgtctgaactaacaaacatgaatgaaataggctatttaatacattctgttttaaaagtaggtttggtcagccatgtaaattgaaaattgggagccaccaagataactcatcaacaaatatgcactatgtactaggcactatatagatgatggtgaaccaaacagatgtaatccttgctcttacagatctcacaacctactatggggccaaaaatatatgtgtatgtgtgtgtgttatacatatatacacacacatacatgtatatatacatatacacatacacatatatacatacgcacacatacacatatatacacacacatacatatgctatgaggaaaacaaacaggtggtgagaaagaattagagtaggggtagaggacagagggctcctcaaatagggtggacagcttgacacaagacactcgagctaagactccaaggatgagaagacagttatgtaaagaaaaggggactagcattgtcagcaggtagctaaggccttaaagcagacagtcatgtgctgcaatgccagcttcaagcgaatacagttactaaagcatatctaaccttctatgtgaatgtagttactaaagcatatcctccaactttccatttttcttttgctattgtttctaccacttctccttttctgttgacaattattttaaatttcctggctaaattaaatgatggcatgaactctggggaaagtaagactacctatgtccaaataatcctaaattccttctagtccttatgactgatcaattcaccctgaagtgacaactatgtcccaattaggaaagagtgtttctttatctgcacttaattttttgatttggaggcttcctgattgctaatcaacatgttgtgtgattacttcaacaagtacttatagaacgttattttgtcactggaaaaacgttctgctgctttctgaactttaggttgctctagagtctaggaagagtgactgtacctaaagcagttcctaattactggacattctcagatctgctagagctacatgtccaattacgagaatatactggaaaaagccctggattagaaatgagaggatgtaggttttagtaccaggtcagccaccttgttaatgcaaatttgagtaaattgttacttcttttaggccttgtttttgctgttttgtttttctgacagtatggtctctgtggtccaggctggagtgcagaggcacaatatcaggtccctgcagtctctacctcccaggatcaagccattttcatgcctcatcctcctgagtagctgggattacaggcatgtgccaccacaccctcgaactcctgacctcaagtgatctgcttgcctcagcctcccaaagtgctgggattagaggtgtgagccactgtgcctagccttacacattgttttcttactggtaaagtgggaatatctagaagttgcatgctacataaattcaaccatatattattggcaaaaaattttaaagaaaaacatcagcttaagagtactaattgagtacatgccttggaatgagcatgagctggaaagaacaaacctgttgttacatcactcattgctgttttcatatgctgctcattgtaaatcttgctcagtggcatgattttagtgtttaaagatttatttgtttgtttgtttaggacaaagtctctacacataatctacttgcttcatatatacatacttatgcatattatgtatgtacatacatgctctcagggctcacatgaaaaaacagccattcaggtgatgtgatttatctcatatgcttactttagagtcaacagggtgttgactccactatacaatactggcatggagaacacataagtcaaagtagacaggacccagccgtaccattggctagggcacaaatatattcacatatgtggagaatgatgtacgtagaaaggtcttcattgcacaatgctctttaataaagatctggaaaaaaaaaacacctaaatgttcaaaaggatagggtagatgaaataatggtacattataaaatggaagattatgcagccataaaaataaggaaataccttaaataataacagaacaacttttaaggtaagtgaacaaataaggtacataatcactatgcatagtatgtaccatttacatagaaaaagggaagaaaaataaaatatatatagtaatttatttgttcttacatgtgtaaaatttttctgaaaaatataccagaaactggtagcactggttgcttcctaggcagaaaatgactgagtatccttttgtaccttttgaattttgaaccacgtgaatgaatgtgttacctatgaacaaaatgacaagtttagatcagcaagacagcagtttgagatgaaatgggattacacccttagtaggaaaaactttttaaagcaggtggtacttctaagagcaaatacctgcacatggaatgttgaaactataaggaactctccttaagagatccatctattccaaacttctcattttatagatctgtaaactgagaccttaaaaattcagtgacttgcataaggtcacacagcagaagagatgggattagatgctagatattccaatatcaagtttagactattaaaaattcagtgacttgtgtaaggtcacacagcagaagagatgggattagatgtcagatattccagtatcaactttagactattatcacaccatcttctcattttctgggggcaaaacagaaccaagtaagtttgggctacattacgagttgtcatgtttttgtttttgtttttttgagatggagtcttgctctgtcgctcaggctggagtgcagtggtgtaatctcagctcattgcaatctctgacccccggggttcaagcaattctccctgccttagcctcccgagtagctgggtttacaggcgcctcccaccgcgcccggttaatttttgtattttttttttttttttttagtagagacggggtttcaccatcttggccaggctggtcttgaactcctgacctcgtgatccacccacctcagcctcccaaagtgctgggattacaggtgtgagccaccacgcccggccgagttgtcatgttttatctaaattttagagtctaatgtataaattaaccttaagccctgaaactactaatttcttgtttggatcactatacggctacacttaaaaatatgctgtgcatacctctatcattgcatgtatacaatatgatagatgcatgatatgacagacacacaatatgatacacgtatttttttctatcctaacacatctgaatttactgaaataactaaaatgtcttaagttacttttttaaatatacacatgcatagcacaagcgtgttgccaaaaatatgaatacaggtttacaattccttaactaaaacccaagggttggatgtgttttagaaataagaatttcatacaatttttaagtgttacagggtatataaaccattatataacacataccaggggccaagggcagcaccccataatcaaacatattaatatagtttcagcaaaacacatgggataaagactatatacagcttctcaatagttcaggtcatattttgctaccaaatgaattttgttgccaagcttaagaagtttttggttttcaccgctttctgaatgttagattgagatgtgggattacagactgtactcatagagtgcttctagaaagcagtcagtcacttcaactctcattttttttttatgagactaaaaaagaaatcatagcaagtagcttttatatcccaggtttgggccaaagacttgtattgtggttaaggaatctaacttagtagaaggtgcacgagctgacatcgtgagtggctaaaatgagagaaaaaaagagaaaatcctaatcatacagaagcactgaactactgcagctgttcgttagttattaatttaataaaagcttcctccctttaaatcatgtgagtttataactggaaataggtcaataaaatttctgtcccacactgctgacaagcgatggacgcaattagctttaatcccactggaaggtactgcactctctctgggaccaggatatgtagaaaaaagcatttcaaatatataggaataaccagaaatgtatacagtattctcaacttgggaccgttactctataatataaacgaaaggggttttctagtcaatctctgctgatctcctgtaccaaagttcttccctttataagtcttgtactaccttttacaagaggaaaaagctctagagcgaaaacacagaacacactaaaatcccttcctttctctttacaactcaagccccgcctccattttgtttctgttactaatttttcttctgaaaaaataccaaatttacactgaaagactaaaattcaactttgcagacaacgttttaaaaaatacaattcagtttggtgatgttgttttgcagtcttacaattttagctacattttaactgaaccaattgttttgttcaatttatgagttaatactcagcaagtttgttttttacaaatagtgtattccattctaaaaatggaagtagcagtggtgaacaagaaaacaaccctctgagttttgtctatttcaggaggaagtactactttctccaattttaatcacaattcataaaaaagaaaaacctaactagctagatcttaaatatacaaatacattaacaatctagtaaagcaacagaaaaaggtaaacaaactaaccagcctatttttgtctggagaaaccccaacaaactgctggattccttggccatttgcattcagaagtaccaaaaactaaaatcctttttactaaataatttcttctacacgagacttgtttcctccacaccaccctatccaaattgtcagcattattccagaatataatcatttagtttgagaccactaaaaaaccccgcagtccaaaataccaattgtggtttttctgtaaagaaatggtcagaaactacaaattgttatcctaggacacagaaccaatcgaccaaaaggacttctggaatatgctgcccccaagatttagaatgcacaggcagaaatagcatacgcggtcacgatgtcccttaagccacatgaccttcctacgaaagcaaaggcttaaacttatcaaatgagaactccccctttctctgaagttaaaacaaggcagggcagctggaattagagcagcagggacagatcggctgttgactagtcagaacgggtcgtggaatgcaaagtccctgcgctttcgctgctccccttaccgtgagaagatctgggagggaggaaaggaggagaaacaccccagaatcctggtagaaaagcccctggcctcgaagatgggctctagggagacagggaggggcagctccgtgtgtgatgaccctttgtgaacatgcactctgtggcagcttcagctccaccgaggctttgggagagcggactacggatgcccggcgcggcccagctgtgaaggccgcgccggcggagagggtccatggcacccccgccggcttcggaagcccttccctctcccacctccgcgggtcaccccaggaaccagcggctcccgaccacgctcgcgcggaccacggaacagcgacgcgcaagcaggtctctttcgtcagcgtaatccctccgcagaaagccgcgcactagttttaatcacgccccaccccctggccgctggcgccacctccgccactcgggcgctttccagcagcttccagaaacgtcgcctccccaaacccagccactcacacatggcgggctcagcagccaccggccccgcccctcctcgtcgccgcagtcgcaactgcgtctgcggccacagggcggacagccacgcctctgcggagggcgaccggaagtgctcacgtcttcaccttccccgccacgccaccgtcctttcaggcccagcgtgcagcaggaaggaggactcttttgccgcggactcaagccggaagccgccttcctagtggagacgcgagtgggggaggagcagtccgaggggaacgtgggttgaacgttgcaactagggtggagatcaagctggaacaggagttccgatcgacccggtaccaagaaggggagtgcccgcggcaggtaagggagaagagggaggggtttctttccgctctcgaaattgggaaaagagacagagctgggatgacctatggggtagtcggcgcgctgaaaggatgggctgggctgggacggggttcaagtgggaaaggttgatgattaaggtatagagttggacttacagatccgtttgggcgcagagaggtgaacgctgaagagaaaccagagtttgttttcgttttccaaggagcgtggagatgggcagggttaacggaccctgcgcctccttcggcttcttagtttgggtgttgaaactcacctcctttggtcctgttcgtctctgattcaagacagttgggtttggtacctgacagggctgggtgcagaaagctgaccctgttcctcggcttccaggtcggttgtggcctcgcttttgacagttcacgtgccgagcctactcgctctcggagggcgagctcaaatgggtgggtttaaggccccctcttcgaacagctgtttccctgggtttctccattttgcacacaggagtgtgaattaagtttaattgaatactttttgcgattcccagggccaccttgacacgttcattgtgctatctaactgggttcatgctgggctaataattcacattaaggcttctggagtataagtggttcacagaagtatgaaaaggggatgttagaagaaagatgctgggggtgaagtagagttgaggaagacagaactggaaagctaggttggtttcacagtacaatgagctttaggtcataatactacctttaggttatattgggctgtttggacggagtttgctgtaatcaggctagagtaaatagagaattttaaactaagcattgacaggctcagacttgtagaggcatcattttgacagtgatatggaagggaaagaggtagagatttgagacctttccaaagaactgtccacagaatttggtgacttactgtgcgaagagggaaataaagaatagggaacaactcaagactttctagtctgtgtgtttggaaggatggagacgcccacatttaagtgagatatgggaaggaggagcagattgtttttgaagggaggaagagcagttacttagggtcaaattaagttgtaaaatcccccccgggattttgtatgtaagtcaaagtgaattgtatttggaagaagaactggggagcccacctctggtattttttttatgtccctcatatggacaaataaacctctggtattaaatgaattttcttttgggggattctatatattcgggatttcaaccaccaacctatctggtttttcccgctgaaatgttgggtgatggaatcaggagagcagatttggagactctttatattttataattgagagagacaaagagaaaaccgtttgatttgaaaaagttttctaggttccctcaggtagatggaaattttcatcaaaaacagtttattcaaggtacatagcctactagtttcccatttgagagtaccgcagaatgatacgacgtgtactgcttctctacgcagaatgaagtataaaattagcaccaaatagtaactttaatttgtcaggtgctaaactttttacatgctttatctcatttaattcttagaagaaactaattttacaagtaagtgtctggaccaacatctgcaggtacaaagcctgaaaagcgtaagtttgactcctacatagttctcttttgtaagtagattataaatagaaccagccaaaggtaataagttgtctgtgcctaaaaagaaagaaaaaagttagcatcagtagttctcaccagaaggggtgattttgcttaccaggggacatttggcaagtcaggaaacttttggctgttggatctagagggtaaaggtcagtgacgctgctaaacatcgtcagtgcatagaacagccttcacaaacaattatttggtcaaagatatttgtagtgctgcagttgagaaatttctgtcttatggttatttcttcaggaataggaaattaagattcgccgatactttctttaaaaagcagttttatttttgaaattattccttggcttgaaaggtttgtgaagtttatatagccgaaccagaatagcgtaattagattttaaagtgaattgtgagccatcgattcccaggagatgggtgtcatagaatcatggattcttggatttgggaaagacttatgcctagaattattttacaacatttctgctaagtggtaattctcctctgccctaaaggtctcctgtatttgattttcctatcattgtgaacccacaattaaaatgctcttaattattttttgcttacactgagctccggtctcttgtaatttttactctgttaaatgtggttctgcaccataggactgcactcaaaacaagcttgccacatatgtaatttgtactaggacagtgtttatatttttgttcagataacaaaataagttaaatgtggtgtaaattagatcatttacaaataataatttgttagcagcttttaataagtagtatttttcccaactggtgaagtattaatgttggtagttgaaaacaataggaatgtatggaatatatggttcactggttcttttgttcctgtcaaatagtggcacaatggatctggggtttttctcagtataatgctggcatatttgtttcaaattgtacatagactctaaaaagttaggctttcaaattctggtcaatatagtttgctttaaatagtagctgcctctactacaagttttatttaatttgttgacaaatgagtctgctatgaaaaccggtcctgttgccagtcactaccctctgttcacaaatttgctgggtttataaatataggtatcattttcacttcaagattataattttagaatatgtttattctaggacatatagccctcaaaatctgcttactatatacgtcttataaaatagcatggttcttttttatagtaaatagaatttttatttaattgtctattgactttttttttccagggttcattgaaaaaatccttagtgatattgacatgtctcaagtgacataaattagccaatgactcggaatgatggattctccgaagattggaaatggtttgccagtgattggaccagggactgatatagggatatcttcactccacatggtggggtatttgggaaaagttagtgaacttattttttgcctgagtgcaaagttttttttttttctctatttttgagacttaaattcaattttgatgttaccagttaacttctaaaaaattgtgtcttccacggaaatcttacagtaatggcgaaagattgttttaatgtgtttacctttctgtgttttattgatacatgaaagtggaaataaaacatagaccttatgatttactgttctttgaaaatatggtacataaattctcccgggtaattgatgttacttttttccttgcaaataaaattgatactattcttaacacataaaatttaatatttaaaactataacataattctttttggaataatagctgtatttaaaggcttatatgcatttcttttgtttgccatgtttaaaataccttgtcaggatacttgtaattgaaaattataattttttctggttacctttccatttaacttttaatattttgatatattctaggaatgtctatattttaatttgctttatttctcttttagaattttgattcagctaaagttccatcagatgagtattgccctgcttgtagagagaagggaaagttaaaagccttaaagacttaccgaattagttttcaagaatctatctttttgtgtgaggatctgcaggtaaagtattaatcttatatagtatatataagatttttcttttttcttttgcttttttattaattgttttaaaagtttactcattttttgttttttagactagatttttaatatgtaatctcagtttgtaagtctgtctggtatacaatgttatttttccacctacctttacttggttgcgtaaagatgttcgtttttattgccatttgatttgcgagaggagaaaatacatttcaaggtttttttctttttttttaaccttttggaggtccttgttagctattagcatatagtagttactctctcatctctttggtttatctttgcaactgatgggaaaagttatgaatttctaatgtacctggaagagtattttggaaattggttagtccaaaaccagtatatatactctgaactaaagagagtatagaatcttgtaaattctaaaagatccttttagaagctctaaatcgcttttagaattatagtaatttgtaccgactggtacggcttttatatagcagctcattaaattctgtaatactccacattttattgtatttgacagtttatgagactgtctcatacacttttaattctcagaactttgcaagatttgtattcctatttcatgaataagaaaataaattgatttcagagggtttgggaacataagatcctgatacagtggcagagctgtggttggaatacagacttctaatttcagatctgtttattccagcaaaaaattagcagttcatcagaattacctggagtgcttttaataaatttctgagtatcacccccagatgctgattcaatagagttggcccagaattctgtggttttgtaacatttgaggatgagtctgatcatcatcagccaggtttggaaaatactagactaaatcacatggttgttaatagatacttatgctgggtataatttgaagtaaagtaatcccaggcgtgtctacaaatataaatttctttatgtttatattcagtaattttttttatgagtgtcactgtttggcactgttgcagatacaatgttaggatacaataataaaacaaaaatttcttgcccttaaggaagttatgtcatagagtgggaaagacagtgaacaagtatgtgtttttctgtcaggtgataaaaagtgctgtggagaaaaataaggcagtagggactggaatgccaaagtagggggagtttgcaattttaaataggatggtgaggggaacgcttcaatgaaaagtgcaattcgagcaaaagcctgaaagaggtgaagagcagtgagctttctaggcaggggaagcaagttccaggaaggccctgagagaatggaggctgcctgtcatgtttgtgctactgcaatgaaagcagcagagcgatagaaggtggatcagaaaaataatgggggagctggaccaagtagggtcttataagccattgtaagctttctggcttttactatgggtgaaaccaggaaccatggcagagatgttggcagaggagtgacataagttgacttcagtgttaaaagcattactgtggctgcactgttgaaaatatatgtaatgggcaagacctgaagcagggagattagttatagtataatatgaattatatttggtccttgtctatggtttccgttacagagctaaaagtcttggaatttcctgaatgataagagtgtcctgttattcagaatgagcctgtttgctaacaccggggttcatactattgtggtgacttaggatggagccgtagatagcctcagatggggcaagtagctggaaagaccacatgattagagaattaacgggttagaacttttagccccacgtacaggcctccaggaaaggagtggaggggctggagatcaagctgtataaaaatatcaagatttggatttaatgagtgggttgctgggggctggtgccgtgtaggaggtggtatgcttagaggaagtggaagcttcatacctcttctgtcccataccttgccctactcatttcttcatctataccctttataatatcctttaggataaaccaataaacataagtaagtgtttgtttgagttctgcgagctgtccttgcaaactagttatgcccaagaagggggagtgggaacctttgtagccagtcagtcagatgtactggtggcctggatgtgggattggcatctgaagtggagggagtcatgggactgagccctcaacctgtaggatctgacatggtctctaggtagataacatccaaatggaattggattataggatacccatttggtgtcctctggagaattgcttggtgtggggaaaaagcccccacacatctggtcacaaaagtgtgctgggaggatagaatatgtgaaaattgtcataatcaaaatggagtcacttgtgttaaaaaagaaaaaaaaatcctgactggccaggcacagtggctgacaactgtaatcccaacactttgggaggctgaggcaggaggattgcttgatcccaggaattggagaccagcccatgcaacatagtgtggccttgtctctacaaaaaaaaaaatttaaattagctgggcatggtggtgtgagtctgtagccccagctacccgggagggggactacgggtgcacggcaccatgcccaggaggtccaggctgcagtgagctgtgattgtgccactgcattccagtcaggatgacagagtgtgagaccctgtctctattaaaagaaaaaaaaaagacaaatagatccaggaaaggctatgaagagagagctttcatgcataaataccaaaatatctcaaaagactctgcaaaaaccacacccttgcacaaaggccatcatgaaatacttctgaaatacacagaaaatacatcatgaaataaatacacagaaaatacttctgcaaggacatctgcccagcaactgcctggtccatctgtggacgggtgtcatccttgttattgatccttgtagccaagggtaattatctcaaaacaagtatgtgatcctccttattttcctttaaaaaccttttgtcttcccttacctccctgaacacacacagtttactatggcatgtgtattcccattggaatactttattcctgaataaatgtcactttctttttagaagcttctcttttctttttatttagattgataagtagaaaggaaaaaaagcttttttccctttggactagttgaaggcagttgcagtattctgggggagagggtggtggcagaggtgttgaggcatggttggagtttatttatactttgaaggtaaagccaacaggatttgctgaaagattgggatatggggttggaaagaggaatcaaggatagttccaagatttttggcttgaaaaattagaagaatggaatcgtgaattactgagctgggaagacttggaagagcaaggttttggggagaagatcaggactgtaagaatagagaagtccttgtccccaggagttaggtttttggctattaaagttagatgtactacatagatttttagttggttttttgttttttgttttttttttttttttttttgagacggagtctcgctctgtcacgaggctggagtgcagtggtgcgatctcggctcaccgcaacctccgactccctggttcaagggattctcctgcctcagcctcctcagtaggtgagattacaggcatgtgccacccagcccagctaatttttgtatttttagtagagacggggtttcactatggccaggatgggcttgatttcctgacctcaggtgatccacccacctcggcctcccaaaatgctggggttacaggtgtgagccaccacgcccagcccggagttttggtttttgaagcattctttttcaagtgataaagcaaaaaatatataatcaagaattttaagtatatactttggaaatgttaaaaaggaacatgagtaatttattattatttttttaatttctagtcagcaatgagagcccagtgtactttatgaagtagattggtttacaccaggagtgagcagacattttgtatgatgcacaaacaaggaatgatttttttgttttttaaatggttaggaaaatatcaaaataaaaaatgccagaaaaaatcaaaagaagggccaggtgcagtgtttcacacctgtaatcccagcactttgggaggccaaggtgggtggattctcttgaggtcaggagttcgagaccagcctggccaacatggtgaaaacctgtctctactaaaaatacaaaatagccgggtgtggtggcatatgcctgtaatcccagctacttgggaggctgaggcaggagagtcgcttgaagccagtggcagaagttgcagtgagccaagatttgagccactgcactccagcctgggcgacagaggagactctatctcaaaataaataaataaataaataaataaataaataaatcaaaagaagaataccctttcataatatgtgaaaattaaatgaaattcaaatttcagtgttcataaataaagttttaccggaacatagccatgctcaatcatttatgtattgttcatggcttcttttgcatacaacaacagagttgggtagttgtgacagactatgtagctcataaaatctaaatatttattatctagccctttatcagtaaactttgctgatccctgtataagtcctctgaatcaaattatttccaaagagttccgttataaaatttggagtttactctgctgtaaattgcaaagaaccatttggaaaacctcttttagtcaggtatttacattaaaatgttccttgatttgtaaacactaatattcaagactggtccaaaattataccaaattgaaactctcaagtgtttttaaacagtaggaagttttaactttttttttttcgtggagtagtctatcattcagcgtttactttggaacatttaattagtcttttttaaaaacccatgaaatttataataaaaattttaaatcattaatgttgagtaatcaaagaaaactttttttgttttctccatttgtaaaatgagtacattattattataatttgtctttggccataccttgttgataattacttatacaagtataagaagacatggtatgttttcctttttcctatttcacaagaataagtacaggaatttacttaagctgctccaaaactcagtgaaagagacaggattaggtttttttcagcattggattttaaatgatactagatggttgcgctgggctaaaatactaatgctttgtgtatatttttatgacttttttgaagacagcttaaaagctttattctagttataaaaatgatacatgttcactgtaaatagaaacaagtcaggtatacagagatacaaatatttagaacatgtggaaagaggcaacaaaattttataaaaagaaaaaagataaaaatctgaaatcattaatttataagggaaaaatcagggcaaggacaaattatattacagattggcctatggtgggagcacagattatatagagaaaagtcagtgaagacacttgcgaagagtgtgggtggaaatcactaagttttgcagtcccggggcctcttatggtttattactgttttgttctttttttttttttaatatgcattcctttggaaccaagggtttattatgttttgaataaagtagaggtgtaagtaggatgcatataccatgatcttgactacttgagattcacaaagggttttcgtctcaggatttttttttctcttaaaaaaatttgtattaatttttaaattgtaaaaaaattcatcaacttaaccatttttatgtatagagttcaggagtattaggtatattcacttgtgcagcagatctctagaacttttttcatcttgcaaaactgaaactctgtacccattaaacaaccacttcccattttcctctcccccagcttctggcaaccattctagtttctgtttcttttctttttttttcttttgagatggagtctctgtcgcccaggctggagtgtagtggcatgatctcggctcgctgcaacttctgcctgcgggttcaagcagttctcctccctcagcctcctgagtagctgggactacaggggtgcaccaccatgcctggctaatttttttttttttttttttttttgtatttttagtagagacgggggtttcaccatgttggccaggctggtctcgaactcctgacctcaggtgttctgcctgcctcagcctcccaaagtgctgggattacaggcttgagccactgtacccggcctctagtttatgtttctatgaatcagactcagtacctcatataaacggaatcatacagtatttgccttttttgtgactggcttatttcacttggcataatggcctcaagattcatccatgttgtagcatggatgaatatacagttaggagttccttttcttttttaagtcttaatctccagtttatttctgtttatttatttattttattatactttaagttctgggatacatgtgcagaacgtgcaggcttgttacataggtatacacgtgccatggtggtttgttgcacctgtcagcctgtcatctacgttaggtatttctcctaatgctatccctcccctagccccctacccgccgacaggccccggtgtgtgatgttcccctctctgtgtccgtgtgttctcattgttcagctcccacttacgagtgagaacatgcggtgtttggttttctgttcctgtgttagtttgctgagaatgatggtttccagcttcatccatgtctctgcaaaggacatgaggagtttcttacttttaaggttgagtaatattccacattatgtgtatgccacattttctttatccattcacctatctgcagatgtttgagttgctttcactttttgggaattgtgaataatgctgcagtgaatgtgggtgtgcaggtaccttttcaagattctgcttttgagttttttttggatacgtacctttttatgatgctttaaatacatatatgctatttttaaaggattctcagttttctgacatatgataggacttaggaagtaatctcaaagcatcatgttgacaggttgttagttgatggtgactgcagctagttggaaagtcagaagaatctagaacttgtccatttatactaaagaatttcatagtaagtgcagtattatgagtgtaatgttcaattggtagaagaggctatctgaggggatttagtgcatttcagttatctgttggtgtgaaacgaatcaccttgaaacttagtcgctcaaaaattttaatggtggctgggcatggtggctcacatctggaactccagcactttgggaggccgaggcaggcagattgcttgaacccaggagtttgagagcagcctgggcaacgtggtgaaaccttgtctctacagaaaataccgtggcaggcgcctttagcaccagctacttgggaggctaaggttgtaggatctcttgatcccaggaggcagaggttgcagtgagctgggatcgtgccactatactccagcctggataacagagccagaccctgtctcaaaaaaaaattttaatggctccatttattatttcacatgattatgtgagttgactagggaattcttacacatcacaccatgtcagctgggacagctgaaatgtccacatggctggcagttggtactagctgctagctggaagttgagttcaaatagtcagccaggggtctcagttattttccatgaggttctctccatgaggccagctgggctcttcacagtgtgatagctgggactaagaaggagtgttccagaagaagggcttgtcctcttgagccagtgcttatcaggcctctatgtatatcatgtgtgctaatgttccatcaaagctagtcacagggccaagccaactctgtacagtgtagggactggctgcaggagggcatgaattaccaggaggtgtagttctctagttcatagggagggccatcaagatagtagtctaccatacttgtgtaaaagaaggcattaattaactattattattattattattattattttagagacagggtcttgctctgttgcccaggctggagcagtagagtggggcaatcatagctcattgcagcctccaactcctgggcttaagcaatcctcccatctcagcctcccaagtagctgggaatacgggagtgtactgccatgcccacctgaaaaagaaggcatattttaaaagcagacctttagtgtagagggttcttgaatttgttatttaaaatattctggtagtttttaaacttaggaaagacccactgattcttttagtgatatgtttacattgttgttatttggcataaattgtgttaatgcacagtaagatttcatgaagtcattaaaattcagccacttggactctaaacccaataaagatgtaaaacagcagtgctatgagatgcatattcagtttcaaaatataggaaacacagaaattactctgtgcacttttaatttgaaaatacttttaaaatgtgtagtataatgtagtgtctgtcccaaaagagtaacattcattatagtgtttctttacgttgttgaaaattttaaattcacttaacattagatttttattaaagcaaaaatatgttttccttattagcttacccttttgtaactcagattaaacccttgattgttcaaattaacctgaaaaaaattattcttttggaggccaaacttttgattaagtagttgtttgtctctaattttttcaaatttatgtgtataaatataacctgtcatcaaatcaatgctaacattctatacatgtttttcatgatatgaaaactataaaacatgaagttatttgaatttgtgtagtttttatcattttatttttactttccagtgcatctatcctttgggctctaaatcacttaataacctaatttctcctgatttggaagaatgtcacactccacataagcctcagaaaaggaagagcttagaaagcagctataaggattcacttcttttagcaaattccaaaaagactagaaattatattgctattgacggtggaaaagttttgaacagcaaacataatggagaagtatatgacgaaacctcgtcaaacttacctgatagtagtggtcaacagaatccaattaggacagctgattccttggagcggaatgagattttggaagctgatactgttgacatggctactacaaaagatcctgctacagttgatgtctctggaactggcagaccttcccctcaaaatgaaggatgtacatctaaactggaaatgccactggagagcaaatgtacatcatttccccaggctttatgtgtccagtggaaaaatgcttatgctctctgttggttagactgtatcctgtcagctttggtgcactcggaagagttaaagaacaccgtgactggactgtgctcgaaggaggaatctatattctggcggttgcttacaaaatataatcaagcaaatacacttctatataccagtcaattgagtggtgttaaaggttggtactaatattttatttttatttacttatttattcatctggagtcagggtctcattctgtcacccaggctggagtgcagtggcatgatcatgtctccttgcagccttgacttccctggctcaggtgggcctcccacctcagtctcccaagtagctggaactacagtcgtgcaccaccatagccagctaagatagtgagatggtggccccactgtcttgcccaggctggactcgatttcctgggtgcaagcacccttcccgcctcagcctcccaaagtgctgggattacaggcatgagtcaccattccagcctacttgtctttaattcttaaaaatattaatgttgagttttgtctcccagcatgtgggaaagatgtcatccattgcttctgtttcctggaggcctgggagcaaggagcccaggaacagtatcacgaagcttgagataataccagttacattatcctgactgcccaaaaggcagtttttttgttttttttttttatactttaagttctggggtacatgtgcagaacgtgcagttttgttacataggtatacgtgtgccatggtggtttgttgcacccatcaacccgtcacctatattaggtatttctcctaatgctgtccttccccaacccctccattccccatcaggccccagtgtgtgatgttcccctccctgtgtccatgtgttctcattgttcaactgtcacttatgagtgagaatatatggtgtttggttttttgttcttgtgttagtttgctgagaatgatggtttccagctttatccatgtccctgcaaaggacatgaactcatccttttttatggctgcatagtattctatggtgtatatgtgccacattttctttatccagtctatcattgatgggcatttgggttggttccaagtctttgctattgtgatttttttttttttttttttttttttaagacagagcctcactctgttgcccaggctggagtgcgatggcatgatctcagctcactgcaacctccgcctctcaggttcaagcaattcttctgcctcagcctcccaagtagctgggactacaggcgcccaccaccaggcccagctaatttttgtatttttagtagagacagggtttcaccatgttggtcaggctggtcttgaactccagacctcatgatctgcctgccttggcctcccaaagtgctgaaattacaggtgtgagccaccatacctggcctaggcagtctttttcaaaactctaagactgtgcttgtgtctcagggtgtcaggataatagtggttagttttaagtgtttaaactactgaaaagcagaatgaagaagtgagtaaaaatcacccataatcacacaacctcctaagatctcttggcacaataagggatatgtttttcattttattctctgtaaaataggatacttatgaacccacctcccaacacaggaagaattaaaacattcccaataacttacatttacctatgcgtttcctcccatcccattctctacctcccccccataagtaatcattatctgaaatgtgtttcatcattccatcttttcttagtttttcttacatgtgtttatctaaacagtatacagtagtctccccttattgtagttgtacttttcttggtttcatttaacccgaggtctgaaagtagatgagtatagtacagtaatatattttgagagagagggagaccacattcacataactttcattacagcatattgttataattgttgtattttattattagttttaatcttactatgcctaattataaaacttgatcataggtatgtagttataggaaaaagcataatatataaaatgtttagttactatccaaggttttaggcatccactggggtcttggaaggtatccctctcagataatgggggatggatggtactgaaccctgtatatacaatgtttttccctatacatacataattatgatcaagtttaattaagagtaaattaaatgtgggccaggtgcagtggctcacatctgtaatcccagcactttaggaagctgaagcgggcagatctcatgaggtcaagagttcgagaccagcctggccaacatggtgaaaccccatctctactaaaaaatacaaaaattggctggctatggtggcacacgcctgtagtcacagctactctgggaggttgaggcaggagaattgcttgaacccaggaggtggaagttgaacaatcacttgaacctgggatcacgccactgcactccaacctgcctgggtgatagaatgagactctgtctcaaaaaaaaaaaaaaaaaaaaaaaagtaaagtaaatgtggctcaacatgttgctgtcagttggaacatttgtttctgatcgtgtcttccacccacaaattgaatgctttttccatcttaacacttatcaggcactgtggccataacttgagcagttgagatgcaacagcaaaattagcacaaatttctttttctttcttcgcagtttcatggataagagatttgttcttagatctcagcaacctcagcatatgatttttttctttaagttgagaactttgacctttttacttagagaagcattttacagcttctctttggcatatctgaattgccagcattactatgctcgtgctttggggccattattaagtcaaataagggttgcttgaacacaagcactgcaataccatggcaatagatcgcatcaccaagatggctgctaagtgaaccacaggcaggagtgtagacagcatggacacattagacgaagggaagattcacgttgccagtggaacacagcaggacagcaagagagttcatgatgctactcagaatggcatgaaatttaaagcttataaattgtttctggaattttccgcttaatattttcagaccacggttgagttcaggtaactgaaaccataggaagcaaaacacggatgaagagggaccacttcgtattgcctaatttagtttgttttgatcttctgggacctttttttcttgttgtaaaaatttatggggctgtttatagttgtggctcattgatttttcattgctacataatacttccattttgtaaatataacagaatattcatctacctgtcagtggacagtggggtttttttgccattataaatgctgctgctgtgaccatttggggggcaagtctcctggggcacagtatgagtttcccttctgtataacaaaggaatggaaaattatagactttcgtgtccaaatttacaagataatgacaattgttttccaaagtggttgtaccaagcaattctcccattaatagtgtatataagaggtcttcctgatccatatattcttcttggtttattttcacacttttgagatttttgctatttgagtggtataaaatggtctgtgatcttgatttgccgtttccacattttgaagaggttgtcggctctatgtgtatatattgctcatatttgttccctcttctgtgaaatgccttttgtatcttatccctatttgttctgttctgttgattgtcacgttttaattgatttgtatgagtttgttccttgtatcattgttgctagagttacatcagatgtgttgctgaatctgctcccagtttgcagcttgtgtttttactttttaaaaactgtcttgatttatagggaagtctttatcttttcatttggagctagtaatgtttgtggctttttaaagaaattattactattcccaaggtcagaaaatcattcacctatattttaactgaaaagttataaagttttgcttttgacattgaaatttctcattcagttggaattcatattgatgtgtggtatgaggtaaggatccatttttttcccatttgcatagccagtttttgtagctccactttattttctcacttgatctgccatgccacctctagcatgtatcaacatatcatgtatgtgtgcagctgttccttaactctcaattttattctcttggttactttgtctaacccagcactcatactttttaaattattatggctaccttgtagggcaagaatcctcacttttattcaacttcttttgaagtgtcttgatgcatattttttctgatcttacttggccatatatattttggggacagatgtgacatcataccaagctttctttgcttgacattgtagatattttcttattcattaatgtgctaaaaattttgagtttggtcatacagtcttttatatggatcttatacatcgtttccctcttgttaaccattcaggctgttactagtttttgctgttgtgaattaacaccaggacaaatatccatatatcttttgaattaattactgactagtttcctaggaaagatattagaatatgaatattaaaggtcttgctgaatacagttttcagaatggttgtaccaatatataattccattttcattatgtagaaaaaatacctcagtgttttctaaccacctttggttagaacattcaagacgttatggttttgttaggtaagaaatattttgtttcagtgtaggttttctttgagactgaacttttttgtgtgtgtcagtcatttacagttttttgcaatttttaaaattcagtttctcacaagcattttgcctttgacttttcttctatttctgctttctctaattacagaaaccccagtgttaagtaggtgacagttcagttgtttgctgcagaagagcagcagttcaatattggaattaactttaattttatgtttttaatctgttactaattttttacagaataattgtagtttttataatctggttaattatatgtttgagctgcattactttgcaatgtaagtttttttttttggcatggtcaaataacaaaaattctggttaatgcttatttcatattacaggagaatccagatatttcattagggaaacatataagcagagtgtgatcaggctgtatgaattatttataagagatgtgagtgaaaagatctatttgtagcttaagagtaagtagagtcagatgcatgtagagtcttttattcaaaataattttcttattaatcttggatagtttcttgtcacagtaattccattttgaagataataaatattaccataaagaagtgatcaaaaacatagatatgtgtgcccaaaggtatttatcacaatagtatttataatagtgaaaaaagaaacaactaaaatgtctggcaataggagaatgattaataaagcgatgtttcagctgaatatagtggcatgcgcctgtaagcccagctactcaggaggttgaggctgcaagatggcttgagcccaggagttaatgaccagcccaggcaacatagcaagaccctgtctccaaacacacaaacacacacacaagtgctatgtttcagtcactgtataataactagccagattttttgttgttgttgttttgtttttgtttttgttttttgagagagcatctcacttgcccaggctggagtgcagtagtacaatcacagctcactgcagcttgtagaaccctaaccctcctgggctcaaatgatcctcccacctcagcctcctgagtagctgggactacgggtgggtaccaccatacccagctttttttctaagagataggggtttcactatgttgcccaggctggtcagtttttaatgaagcacatttgtgtagacaaagcaggatgtggaaccggataaacactatgttgccactgaagaccccttcaaacccctcaaaaatgacatagaagggaaatatgagatattagtttgggaaataattgtaactttattaagactccttataaatttatctgttcctatgacctggctaagttcaataaaagttacacagagtggaataaatggttagacatcatttgtagtataagtaattgcacataaggaggtaactttagctgttttagagatagacatagtatctgaaaggttagttattttactagacctgtgattatttgggtgagaaaggctttcactgagattttacccattcagtaagtactaatgatattgtgctgatagcatatattaagggaatatatggtataccacagagaaagaattaaggaaattttgtgttttgctttttgtctgtttgcaaaacttactgactcagctttcattcttgggaatgtgtcagttttctgtgggaagatatacattgatgaggaattgataatgttctctgtattttcttagatggagattgtaaaaaacttacctcagaaatatttgcagagatagagacctgtctgaatgaagttagagatgaaatttttattagccttcagccccagcttagatgcacattaggtaagtaattggtaaaacttacttgtattatactcatctaccatatagaaatatgtacctcataaggaaatataatactgtttgattaccttggatgatcatattcttgggagagagaatctgagtagtttgacttaggaatctaccactgggtaagttattgtagggcagagctgttccatataaatatgtaggctggtgttccacctcttgagagtgggtgcagttctcagaaccaggagaattttagggggcatatcattagttgcttctctagtacgtttcctagtagacagatctagcatttttaacctcaattgtgcattaaaaagcaccgagggaatttaaaagtaaatgccaatgctggggcatttgaattaggatctcagggatggggctcaggaaatcagtaatttttagaaaccccacatgattgttatatgtacccagggtttagaatctcatctaaaccaaccatagtaattctacttccctaccagtgattggtttaggaatgtccttgtggtagagttttggccagtggatattaagagaaatatgctgatggccttttgggaaagcttcctcgcctttagaaagggcacaaggatgggacctctttgttctctgtgacttggtttttggcctgtgggagtggcgtgcagcaagtgagctagagagtctgtccaaacctttctaaatttttttagtattgcgaaaaggagctgcggggtttttttgtttgtttttgttttgaaagggctttttgttttatttttcttgtatccttgtattaactcttctattaatgttatagtagcagaatatgatactccctattagtaataacccatattatgtaaaatatcagtgccttctagtttttctctcaatgagtgacatttaacttatattaaaaaatgatatttatattttataataaaatcagttgttgctactgatttgtctagcatgtacaaaagacaccatgcttccagatcattataaaatatgatattttataatatatttacaatatatttataacatatttatatacttagaatatattttataaggctgggcttggtggctcatgcttgtaatcccagcactttgggaggccaaggcaggcgtatcacaaggtcaagagattgagaccatcctggccaacatggtgaaaccctgtctctactaaaaatacaaaaattagccgggcgtggtagtgtgtgcctgtagttccagctactcgggaggctgaggcaggagaatcgcttgaacttgggagacagaggttgcagtgagctgagatcacgccattgcattccagcctggggacagagcgagactccgtctcaaaaaatgtatatatatatatatatatatatgtgtgtatgtgtgtgtatgtgcgtgtgtatatatatatatcgggaagcatggcatcttttgtacatgctggacagcttttgacgtacttctttgactcatgcttctgccccctaattttcactttttttcctacattttattaaaattaatatataatagttgtatatctgctttatttttcatggacttatacatacatatttattctgttcttataaaagtctgatttttcgtatgccaaatttctgacatttcctcctctaggcctgaagaactgttgtaatttatgcatcagataggccctcagatggaatgaatattcttttttctttatatcaaggtgtaatttacatatagtaagaccgtttttaagtgtgtacagctctgtaaccctcactacaatcaagatataggactctgtcactctaaaacttctcaccaggttcatcacccccagccactgatctgttgagcgaatactcatttcaaaggagctttttccgtaagatccctagagtttagatggaagggctttcgtggtgcatttagcagataccatttcccttctagactccctacttcagttcccagttgaattaaagaatggtttctcccccagcctgagtcactacccttcttatccctgataattatttttggaacaaagttacatcttttgctccacctccgccatgggcctggttttctatgtaacagaaggaatttttaaattattgttttgtgtaatcataataattgggcaagcatacagctcttttcagtgcaggaggattcctctcttgttttactgcccattcaaggataggtgctatattttagctgaagatcttactaatgaaatgctctgtaatcatataacttatttaaagatgtgttttgagctctttcataatattttaattcatggagaactttatgtattttagacctgaagattttatattgtcattatgaaatgtaaattgtttgctttttcagttaatatatagttacaatagaatacggatttaaaggctgataatgaattacaaaattgtgctatatgacatactgtttatgcatacagtgttgcatattttcatttctaggatattgatttgtatttctacttacaaaaaaactttttaaaacttattttatggctgggcccggtggctcacacctgtaatcccagcactttgggaggccgaggcgggtggatcacctgaggtcaggagttcaagatcagcctggccaacatggtgaaaccctgtctctactaaaaatacaaaaaattagccggacgtggtgtaggtgcctgtaatcccagctactcgggaggctgaggcaggaaaattgcttgaaaccaggaggcagtggttgcagcgagcagagattgcgccattgcactccaacctgagcaacaagtgcgaaactccttctcaaaaagaaacaaaaaaactttttttaatgtttttgttcaaaagtagcagtgagactatcccgcaaaggtgactactaaaatagcctttgtaactactgatatttatagaatatgcttagggttagggtataactcgcttgtattatactcatctaccatgtagaaatatgtacatcataaggaaatataatactgtttgattaccttggatgatcatattcttgggagagagaatctgagtagtttgacttaggaatctaccactgggtaagttattgtagggcagagctgttccatataaatatgtaggctggtgttccacctcttgagagtgggtgcagttctcagaaccgggagaatatttaggggacatattgttagttgcttctctagtacttttcccagtagacagatctagcatttttaacctcaattgtgcattaaaaagcaccgagggaatttaaaagtaaataccaatcatagggacatttgaattaggatctcagggaaggggctcaggaaatcagtaatttttagaaaccccacatgattgttattgcttaggtaataacacctactgtctaccttgtggtcctgccaaggtgactgttcctggccatgttccaggcaactgtagttccaggctagggggagaactggaccatggaagtgaggctctgtccagggtaggggaagggatggaaggtgactgttcctggccatgttccaggcaactgtagttccaggctagggggagaactggaccatggaagtgaggctctgtgcagggtaggggaagggatggaaggactcagtctcttgggccaaatcggtaaggcagcatctaagctcctctgagaataggaaggagagcaaccaattggaaaaagaatgggaaacatgtagattctcctgcttaccttactttccagtctcaaagctggaagccagcattcactgttcagttattttcaatgacaacaagattcaaatcttcagttgtaaagttgttaaaggaaaggattagactgaaaagttaagaagaacggtagatgaagagtccaaagagttgaggctggtcatttaaccattgtgtggccacgccctctccacaggtggaacaagatgatcagaatagaaatggccaattctgatgtgtttctacagtgtttcactgattacattttttaacatctgtagcaaaccatttccataatttttttttttttttttagagacgaggtctcgctctgtcacccaggctggtatgcagcggcatgatcatagctcactgcagcctcaaattcctgggctcaaatgagcctcctgccttagcctcctaagtagcttggactacaggtgtgtagcaccactctcagctaatttatttcattttattttttgtagagataatgcctcgctatattggccaggatggtctcaaacgttcatagaaactggttttaggttcctagaggctggcagcaattctcagaggtaacgcaagcagtcttcctgccttggcctcccagtgtgctgggattacaaggtgtgagccaccacacctcatcaatttttgttttaatatactctaaggcttatcatagttccgagatctttttttttttcctgagaaatctagaaagatggaagacagtatgggtcttttgtggattttttgtcctaagaaattttcataaatgtctgccaaggaaaaggaaagagatcaaagtggtaattaaatctttaggatggacatttttagaaaaatgctttataaacttcccctctcccaactctgagtgacttattgtgtcatactgtattaacacatattcatgctgtaaatatagtaagaaaagacaatagttcacaattttggtttagtttttgccattattgattatgagcagtaattcttccttttctttttgaaggtgatatggaaagccctgtgtttgcatttcccctgctcttaaaactagaaacccacattgaaaagctcttcctatattctttttcttgggactttgaatgttcgcagtgtggacaccaatatcaaaacaggttagtttcttttgttttttaaaatgggttcttctagtttctccaccactaaggttaagagaacaatttgagcaccagacactacagtttgcttgcttctttaaactggaagggtcaaaacctcatcgtttgatagactgctagtaggatatttcctaaggagttcttcagtgggaaatagggacgatgagaggaataatacacctcccttctccagagtccttgctgagtagaatacctctcagaatgccatgaaactgtaggcatttttgtttattcctctattagaaatgaggggttttgcttgtttactttaggtttctaacattatagacactagttttaggctcttggaggctagcagcaattctcagaggtaatgcaagcttccccatttcttcccgtagtcctgtgaaagaccagccacctccagaagcctacacatgagtcttctcagccatactttctgcttttcctaatgcctctcagcagcgtattagaaaggccatgatcgatgtacctgttaccttcaggctttgcataaggtgtatatgaaacataatgaatttcgtgtttaggctcaggtcccatccccaggttacctctttatcttggagacacttctggtcccatacatttcagataagagatattcaacctgtacccaccacgtaaggagaggaataggttttagaagaggagtcagggaggcaaggtattcccagagggatattctcacttggtccatacctgagaaagttgctggctggcagttaggaagatgaccagactggctcaattgttcgtgtattcaaattattacaatagaaataactctttccacccccccccgccctttttttttttttgagttggagtctcgctcccgtcacacaggctggagtgcagcagcgtgatcccggctcactgcagcctccacctcctgggttaaagcgattctccttcctcagcttcctgagtagctgggattacaggtgtgtgccaccacgcccggctgatttttgtatttttagtagagacagggttttgccatgttggccaggctggtcttgaactcctgacctcaggtgatccagccacctgagcctcccacagtgctgggattacaggtgtgagccaccatgcctagccacacttttctttagcttaagtgcttaagttagaaaacttgaagtctctctaagttactcaagtaaaatgtgagataaaaatattacttttgaaggccgggcacagtggctcacatctgtaatcccagcactttggtaggccgaggcgggtggatcacgaggtcaggagtttgagaccagcctggccaacatggtgaaacgctgtctctactgaaaatacaaaaattagccgggcatgatggcggacacctgtagtcccagctactcgggaggctgaggcaggagaataacttgaaacccgaaggtggaggttgcagtgagctgagattgcaccactgcactccagcctggtcaacaagaatgacactccgtctcaaaaaaaattaaaaaaaattacttagatattcattatctaaatatgaaatcctttttaggtatttaaggagtagtcaaggagagttcagtctgggaggatgctccagggaatgcaggcaacaaaggttttgttttttttttaactggttaactcagatctactagaacagggtaagggaggccacagagtagacaccatgagcaaagctaaccctcctgagttgaaaaaattatggacgagaagttatcattgaaattaactgttggcagacatatccaaagaatatcgcaaggatttggtccctttatgcatcctgagacagatgaatgtgtggaatggcagctggtgggcaacagagcgatattggcatggtggtgatacagggaaatagtttcatcgtgttaaaagccatggaacaaagatacataatggctgctctgcagaaaaatccacgtcccctctccaaagggcctgttttactctgatgtaaaaattgggtcagataaattttcatattaagctttttgttgagtaaacttttgtaatagtccccaaaactcccactagaacagggtgagaattaacgttttattcatacctaggacttaaataatttagtgtaagcaagtgagtatgagaacacatctgtttccagtcttctatcattgctttatataaattctctggttttctcctcacagtaactcagtgaggaagatcctagtgtcctcatttggcacgtatggatatgacagcttgaaaggggttagattgattcccaagatgacacactgtaagtggcagagtcaggagacacacttaggctcttctggcctctaagactttcttgctcactgtggtatactccttaatcactacctgggttttaaataatataaataaccttgctgattaaaatcagcttaattgtagcttctctggaatccatatcttagttgtttgacagttttcggttgagtgtcttctgtgtgttaggaactcaggcactggaaatagtgtatctttgccaaatttactaattaggtagagagataatacacgaacacataatagaggtccagtgacttcgtaattaatctgatctttgggctgcttaacgttagctttgaatgcaagatgttaaatgcgttttagagatatatagcacaaactgtgagagctcaagggagggaagccactagccgcttttgtttgcttttttgttttttaaaaataatcttactttgttctaaaaataaaagtagttatagagggaaagctaaaatgaagtgacgttttcttaaatatgttttaatatgtcataacttaaaacttatttccacttaatctgaaggagaactgtccagcaaattcctttgtttttgtgaagctgtttttagtgccagcataagggctttttactcaacttggaaagtgtaacccagagtcagttaaaaacatagtcttcagaggcagatctcaggtctgttatttatcactgtactctatgtgtcactttccccatctgtaaaatggggataagaatagcacctgcctctgagagttgtttggaagatgagtgtccagtgccatgccctttgcacatagtttaagtgttcagaaatgtcagatgtcatgtggagaattaacacttacttgctgagacagtctcctttttataaactaaacagtaggagcctttacataacaattatctttgaaaatttaagaatttagcagaaatcagtgcatttgttgatatctttatgttgctttgcttttaaaatgttaacctccctgactactgatgtttttaacagacagtgcttcctcacaagatttataagtatttgctattgtttagaaaggaagcttgtatctcttaagtagctgctctttaaattacaaatatttttattaaagtggatgcagttgaggtttagtgtacatctttaaaggtcatctttttagatggcgttgctctcaagtattcagactaaagtgcaaatttagaacttgtgtaacctgtgaaaacaaaatttgttcacaattaatgctgtgtgtgtgtgtgttttttttttaaggattaaaaaaagttaagttgtatgtattcctgattttatgtttggaaacatccccttttcatttttggttgtctgtaatggctagccagtttgagttatttgagtaaggggtgagctcttaataaatttgacaaccttagaacagtggttcttcactaagggctattttttcccccttgggacatttggcaacatctacagacaactggatgccgttactggcatctggtgaggagaggccagggatgatgcttaacatcctacagtgcacaggacagtgcttcacagcaaagactctctggtgaaaaatgcagtgataccattgaggaaccctgtctttttttcttgcttcatctcatagttgaaagatatgggaaattaacatggagcatcttcacagagcttctttactagaggtagggaggaacattgccatattaacatgatttggggaaataagaaagtatgaatcacgaaaaaggggaggaatacttttagacattggtttaaattaatgtaaatgcatttaacgttaatgaatttgttatgtcatttttttataggcatatgaagagtctggtcacctttacaaatgtcatccctgagtggcacccacttaatgctgcccattttggtccatgtaacaattgcaacagtaaatcacaaataagaaaaatggtattagaaaagtgagttaaaattgtcttataatttttagtacaaaatgaaggtggatttacatttttcttaatgtgtaggattgaaaatggtgacaacaacttacctttctgaaatttgagttaacatatatttctgggttgccagctgcctcgctctatctggccagtgagcccactgtcacggtgaagccactgaaaagccaacttaggctgactctctggccccactctcctagtgtctttccttctttttgccttttttctccctttaaggatatcaagcttcagtttttctctcctctgccaagtgtatggagtttctagaattctgggatttccttaatcagatttcaagaactaagatgattcaaagataagccacaggctcatctctctgaatttccatcttctcctagatctcagcatgctaattcctcatcatcttgaaagctatctagtggccttgagcagatatattttcattgtattttgccagcttttctgtttgtcctcagttggggaggttggtcagcattaccttttccagtattaccagagaaccatctgtttaaactcacaggtcagttccatctcaggccgtttccctctgtctcattaatgcactcacacatgtacacaacctctctactcttcattttcagtctaatcgtacattaaggaaatgttttgaggtctaatttgatgtaataaagaaccgggaacattaacctttatgcccttgaatgtgccagaaacccttcagaatctttcctaaaggtttattctcattgaagtaataaatcctcagtttatcagtgcttacaggctcaaaagggaaaaagggcagtagtcccctgttccctcctccaggtatctactttaaaccttcaaattaaggtagtatttacttttacttttcaaattgatgtgcctattctaccgtaatgcagtctgttctccttttatagtaattgagactagggttctcacaccaacacctgggccccatctctgtttagcctttccctgtcctttcaatgcaattgcgtatttggctaactcagtactcggtgtttgcattgttattaatatacatgtgttattccctcttcagccaagcagtatatatagttaggtttcacttttacaattcttatttttccgggaattgttatttgccttgttttcatttgttttattatgtactgtgagtttttgccaaatactttaaagacttattaataaattttcaatactcagatgcttcacagttttttactctgttcctctcccctttttttcctggaactctttcctgccacctttcactctttgctgcagtctgcgctggttcctctctgggcctgcagcatagggtgctctttattatgtacacacttccagtcactatcgtagtttttagcccaaggcctcatccccacattctatcacatctgttgcccataaatatccagtcctttaggggttctctgggaaaaataagctcttctttgtcatcaacatatgcactccgtagtactcatgtcttcactttgcccgttctgctgggtaaggtgccacttctctgtttgctttctgtcctctaaatatttgacttcttatttgcttattttcctttctttgtccttttggactcatatcttttttgcccctcactattatttgatagcatttgtgtaggagggcgaagtgggaaggaagaggaggtgtctgtatctgtctgaagattacagaagtctgtaatctgtcttggctgccaggtgtcagttttgagatgtaaatgttgatgatgaggtgaggagaagagcagcagagcatggggtctgccatcctgccttggaccatggcctgctttaggctgcttggtgtatatgatttcatctagctgttcatacctgctttttcctgtgccccagcactgaacatagactcgtaccattgttttgtgtaatctgttaattggttgcactgcagcatatatattttttaactatacaaataagttgcttcccttaaagattcatgctctgatctggaaatggattcattaggtaaaagtcttttaatggaaaatgtgttttgagttccagtgggccaatttatgagcagaatttataatgtgggcatttcctgttttcttcaaaagtaaattgaactagtgtatgaagtttcacttaaattttaaatgccaaggtctttatataagtcctttgtgtttttttaattttgaaatttgtataacttgatttgtttgtgtctaatggaatttagaaataaatttaatatagtttttagggctaacctaaaagtaattgggttcatcatggtgtcatatgtaattaaaacatatagaatcctaaaaactaattaagttccttggacaccttatctcacataacccacatctctaatgtctccccattgggaaaagagtccattgataaatcaggtgaattatgcctagcgggcccaaatctgctacttttctttaagttgtttaggagttacattcagaccatggtgacatggagcaccaagaacttagaatcagatttcattttacttgacaaactcttgaaaggtcactgccacagtctctcttgagtgcaaggctatggctatgctttgtagcacagggacgcgatatttctctgctatctttgggtagcagaggttaacacagctcccttgtgctttctttctctcttttctattttcttttcttttcctaaggatagatctttaaataggaggagtttaaccccatgttaggtgaattcaaatggatcttagcctgatgtctcttgttctcttttggttccagtttggttaattcctttcatccaattttccagtggttgagggagaacctaacttgctctcctcgactctgagcatcatccttcactgacagttcaggcattgtgggtaggaagaagtctgagaacaaaacctagggataaagtttagtagagatggggtttcaccatgttggccaggttggtctcgaactcccgacctcaggtaatccacctgccttggcctcccaaagtgaggctggaaataagacatgctggaattgtaagtaggacactagagtctaggggaatcaaagaggaaaatgaacagaaaagggaaggggaaggatattatttgattgactccaagatgctactgtttgtaagttttaccattttaaaaatatgccattaagaaagaaatgctggccgggcatggtggcttatgcctgtagtcccagcactttgggaggctgaagcggacagatcacctgagactaggaatttgagaccatcctggccaacgtggtgaaaccgcatctctactaaaaatacaaaaatcagctggatatggtggcacatgcctattgtcccagctactcaggaggctgagacattagtactgcttgaactggggaggcaaaggtttcagtgagcagagattgtgccactgcactccagcctgggcaacagagtgagactgtctcaaaaaaaaaaaaaaaaagaaagaaatgctgcttatttaactgtgttctgtcaatgttaaggtgtatcccgacttcagagatgttaacaaatgggaaaaaatttggaattcattaggcatttggaacttacaaagtttcggccgggcatagtggctcatgcctgtaatcactttgggaggccaaggcgggtggattacctaaggtcaggagttcgagaccaatctggccaacatggtgaaaccccatctctactaaaaatacaaaaattagctgggtgtggtggcatgcgcctgtagtcccagctactcaggaggctaaggcaggagaatcgcttgaacccagggggcggaggttgcagagagctgagatcgtgccctgcactccaacttggacaacagagtgagacgccatctcaaaaacaaacaaaccaaaaaaaaaaaaaaaatttcatagttacagaaagtagtatggaggccataccgagattttcgacatggtagtaaaactctgcattatggctctgttctgcatcatctctgttctgcatcgtttcactccacatcagaccctggatagctttggtgtactggtcgatcttgtggcagtaaggctagtgtaattaagaggatattttaaaacttaacatataattgctctagttgttgtctcttttttgctggttaagaaaatcaaatttctatcctatctgaatctcatagcagactttggagatttctgacaagtcatttcttactacctaggggaatgtacttgtactcagctagagtctgagtatcttctacatccagggaattgggctgagtgtggattttggtcttggcagtttttacttttattaatttgcaaaagaatagaagacttggaatgtacaagaagcataaaaatgtgtcaggtggttttacatgcgttatttatcacgttaatatgtcttaagatattttccacgtgtaaacttatgtaaaggcaggaaactagtgagatttcatattctagggatcaagagattgttttagtaactagcctcagaaagtatcttgaaaggtattatataaggtcaaggaactaaatattagtaaagagtcaggccaggcgtggtggcttatgcctgtaatcccagcactttgggaggccaaggcaggcagatcacttgaagtcagcagttcgagaccagcctggccaacatggtgaaaccctgtctttactaaaaatagtagtgtgtggtatggtggcgcatgcctgtaatccagctcctcaggaggctgtggtgggagaatcacttgagcccaggaggcggagattgcagtaagctgagattgcaccactgcactccaacctgggtgacagagctagtgtctgtctcaaaaaaagaaaaaaaaaaaggtcagataggtgcctaaagcctgtgtgtctcgctatgagaatacatctcaagttttactgtggttcattgattcagacatgtagttcacattttaacctgtctgaaatggtaatatgtgaaattgatgtcatgatatagtttaattggcagcatgttttcatagtggtacattttataattagtgaaatcttagatttgatgaaatagatatgattttttaaagtgggaaagtttagtgttatagacagtttgcaggactttttattttgtaggtacttaaattttgaggacttaattattctctaataaagtgattgacaaggattaatgtataaattataccttgtcagtctgaacaatctgcagtttggacattgattcaaattcatttaggctgaataaattttgataaactaagtaagttttgacagctatttaaatattgggaaaggggatattcaacatttttcttacatcctgagagctttgttaaatttagttatttgagacccattgggttctattttctggttcagcatgttgctgtaatggtaaaatacaattttgaaattatagttgtcttgaagttaataataaattgaccaatatgttgtatttttttctctacttagttacaaattgaacttttcctaagtagaacttttaatttgacaggccccctttgcttcctgaggtaactgaaataggccaaattaatgcttttttgaatatcttaggtttgttgctttctttcacatgttacctaccccacttaacaaaagcaattaatctcagcacttgatgccaaagaaaattctaaaaggtctggattttttccttggattttacaaagtagctacaatgggacttttaagacaaagctgcattgctgcttacagagcaatttttgtttaatggtctgtgttagagtcatactgcatgatgacttccaactgtctgggataccattctgaaaagggtttagtgttacatacttcttagagagagttctccatttctaattaaggcacacatctggaggtgctcaagaaaaattagtgcagttagccttggaagtgttatgtgtgactagttcacttcagacatcttttgtataatcagacacatggcattaaatttatttaacttctcttgcttttctctcccacagagtatctcccatattcatgttgcactttgtagaaggcttaccacagaatgacttgcagcactatgcatttcattttgaaggctgtctttatcagataacttctgtaattcagtatcgagcaaataatcattttataacatggattttagatgctgatggtaagtgtttagaggttttcttttaagataattggcatagaaactaaattctagcatgtggggactttttggtttttgttttataaaaaaagacaaactttgtcctgactctttctctctccattctcgcctttgccttctgcccctcctcgcatctattaaaagtgatggttttagtatcctgtctcattttttcctttccttacatcatgtattataggtaaacacatgcgcatgtgtgtatttctcttttagacaaaggatgagattactactgttagctcagtttttttttccctacttaacatctttgcttttattttttagacatatttctaagactattaaacattagacttacgtagcccttctgtcattgtgaaatacatagtttactaacagctaccatcaagataaagcctttatttaaataattaaacttcttagtggaaagctaagtaagcacagtttatggattttgggaatttttgccttgcatttgtctgatatggtaaaatattgagtttgtttttctcataatgttcactttgtcttagacaagataactcaatccccttaaagggttgtatcaagccattgataagggctcactttgatataaccattttctgttatttagacactctttcacacttcctattttcctcctggggatggtttgaatggatgacacaataccatattataaaagcactttacaaactgtaacttatgttataaatgtaattattaccttaaggttttaccctgtttcagatttgagtggaagtagttctttacaatacaaaacaacttattttaactttttttgcatttcaaagaatgatcaatccacttcaggtgcagcatggtttccaaccctgacagcatggaagaatcatttatttagcttctaaaaatgtgcaggctgtaccctagaccagccttggggattaggcccaaatatcaatgttgggtgtttttggtattggtttttggcccgcctacccgcccttccttccttcgttcctctctctcattctctctctctctctctttctctctctccttctttgctccttcattccttctctctctctcttttttttttgagacagcatctcactatattgcccaggctgttctcaaactcctgggctcaagtgatcctcctgcctcagcttcctgagtagctaggactacaggcacatgctatggcaatactgttttaaacattgttttcaaggctccccaggtgattccagtgtgggtcatgtggtagagaaccactgacacaggcaaacaaaggatacataaagttgtctatttaatgggtaggtgcaggtagtagataagagtgtagccacataaaccacatgcttagtgaacggttttgttttgtgtgtatgtgagggattagcatctctgagtatattttgttttcccttttgaaacttatcagagaattcatatgtctgttatgtgactaatgctcacattaaaaaaagttatgtgactttttttaattcatatgtctttttaattcatttattcattcatatgtctgttatgtgactaatgctctcataaaaaaagtaatgctcagtttactttttttatatcagatcatatatatatgtttttttttttgagatggagttttgctcttgttgcccaggctggagtgtattggcgcagtcttgtctcaccaccacgtctgcctcccgggttcaagtgattctcctgcctcatcctcctgagtagccggaatacacgcaggcgctaccatgcccggctaattttgtatttttagtagagacagggtttctccatgttggtcaggttggtcttgaactcccaacctcaggtgacccacccgcctcggcctcccgaagtgctgggattacaggcatgagccaccgcacccggccatatcttatattttaataaatattttaatttggtctgtaaatttttctttttggggaatgtgttttaagtctgtgttgagtcctagacatttgttgttctcagatagtcactagtgataccttaacattaaccagcctgttggcaactaaattggcctgaagtgacaactaaggaaaggtctctttctcctttcttaatctttgcattccttaagattagttctttgtaggaaggctttgaagtctggtggcaagtaccctttatccctcacaatcttaagataaggtctttctgagcattaaaaagtgactgtgggagatatgtcaaatgagttttctgtgtgtgctctgagaaatctttttttcaaaaaaggatagatgtacttgtataaggaaaagagaaactgagcgcactttcaatatttaagtaagtgtctctaacatgttttgcaacataaaatgatgaccactgtgttggtcattacttctctactgctaaaacaatgttttctaaaataatatactccttagaaaaaaatatagtgctttgggtgtgcactgttgtaatccaaggaataggaaatgttttgtagtaagtgcgatggtgtttgacatcgtgatttattaatttatcacatttggtttcatagaaatagagtaagctacgtatttgctgtgccgcaattaccatgacattacacttgtatctatttctgtttcatagatgtgtagatattgatatatacagtggaagtatggattgttttgataagtttctaatgaaagtacagatatttgttgattatttattaagaaaggttgttactcatccaagcccgtggttagcttttcccaaattatcatgtggtagtaagtaaaatgtaaagaaatataccctcccttaaccccacaccacctgttagcacctagccaccttcctttacttctcagccgtactttttgtatttttttgttgtagtggtaaaatataaataacataaaatttaccattttaacatttgtaagtgtacaattcattggcattgaatacattgtgtgcaaccaccatcaccatcaggactttttcatcaacccaaacagaaactactcattaaacaataactccgcatccttccaccccaaagccctggtaaccactattctactttctgtctctgtgaatctgtctattctagatacctcatagaagtggaatcgtacattatttgtccttttgtgtctggcttattttactcagcatattttcaagattcatttgtgttgtgggatgtagcagaatgtcattcctttctaaggctgagtagcattgtatgtattatccatttatctgttacggacatttgactattgtgaataatgctgttgtgaacattggtggacaaggaactgaaagtccctgcttttcattctttttggcataaacctacaagaggaattgctgggtcttaacggtaattctgtgtttaatttttggacgaactgccagactgtttccacagcagttgtactattttacatccccaccagcgttacacaaggattccaatttctctacatccttgccaacatttgctattttctatttttttttaataatatccatcctaatgggtgtctttttttttttttaaaggaatggtttaaacaggttaccttcttactcctcattcatgctttagttgactacataaggacccctctccctattggcaccattgaaattgttcaggcaaaaataactgccagcgacacactgctttaagtaatggacttttcccaagttttgtattaatatttcagtatttggtagtgcatcctactgctagtttttaaactcttcccttgtcatctatcatctcattctctcttgacaaatgtgaaaatggaagctcagaaataaaacaagaattaaaacgaatagtgatccttcaggtaacaagcttcatttatcatgaaaacatatatgtatgaaacattctgttttctgatgttattggataaattaggtgataaccaaattctaagttccaaaaattaaatatactctatctaaggactttaacatggcagacaatggtgacaaggtcaagaacatgttttagagtcttctcctttggtcggtattcaatgatacaacagttgaaaaggccagaagaaagttaacctaggatggtggtttttgaatatctaactttcacttctttcccatcttccaggaagttggctggaatgtgatgacttaaaaggcccatgttctgaaaggcacaagaaatttgaagttcctgcttcagagatacatattgttatttgggaaagaaaaatatcccaagtgacagataaagaagctgcctgccttccacttaaaaagactaatgaccaacacgctctcagtaatgagaaaccagtatctttaacatcgtgttctgtgggtgatgctgcctcagctgaaacagcctcagtaactcaccctaaagatatatcagttgcccctcgtactctttcacaggacacagctgtaactcatggagatcatttactttcaggtccaaaaggtttggttgacaatattttacctctgacacttgaagaaactatccagaaaacagcctcagtttcacagttaaattctgaagctttcctgttagaaaataaacctgtagcagaaaatacaggaattctcaaaaccaatactttgctatcacaagaatcactaatggcttcttcagtatcagctccatgtaatgaaaagcttattcaagaccaatttgtggacataagttttccatcccaagttgtaaatacaaacatgcagtcagtacagctgaatacagaagatactgtaaatactaaatctgtgaataatactgatgctactggtcttatacagggagtgaagtcagtagaaattgagaaggacgctcagttaaaacaattccttacaccaaaaactgaacaattaaaaccagaacgtgtcacatctcaggtatctaatttgaagaaaaaagaaactacagcagattctcaaaccacaacatctaagtcattacagaatcagtctctgaaagaaaatcagaagaagccatttgtgggaagttgggttaaaggcttaataagcaggggtgcttcttttatgccactctgtgtttcagctcataatagaaacactataactgatttacaaccttcagttaaaggggtaaataattttggtggctttaaaactaaaggtataaaccagaaggccagccacgtatccaagaaagctcgtaagagtgcaagtaagcctcctcccatcagtaagccaccagcaggccctccatcgtctaatggcacagctgcccacccacatgctcatgctgcttcagaagttttggaaaagtctggaagcacctcatgtggagctcaactcaaccacagttcttatgggaatggtatttcttcagcaaaccatgaagacttggtggaaggtcagattcataaacttcgtctaaaacttcgtaaaaagctaaaggcagaaaagaagaaattagctgctcttatgtcttccccgcaaagcagaacagttcgaagtgaaaatctagaacaggtgccccaggatgggtctccaaatgattgtgaatcaatagaggacttgttaaatgagctaccatatccaattgatattgccagtgagtctgcatgcaccactgttcctggtgtttccctgtacagtagtcaaactcatgaagaaattttagcggaattattgtctcctacacctgtttcaacagagctgtcagaaaatggggaaggtgactttaggtatttgggaatgggagatagtcatatcccaccaccagtaccaagtgaattcaatgatgtttcccagaacacacatctgagacaggaccataattattgtagccccaccaagaaaaatccatgtgaagttcagccagactctctgacaaataatgcctgcgttagaacattaaacttggagagtccgatgaagactgatattttcgatgagtttttttcctcctcagcattaaatgctttagcaaatgacacattagacctacctcatttcgatgaatatctgtttgagaattattgaattaatgcttgttaacttttttcatataatatttattattattagaagaacttacaatgtgttcaggtagtgtttatacactggacttgtgtaattacttgtgtaataaccatgaacaaaatgcaaggtttaacctttggttctgcccatgaagcatgtaatctttcttacacattaaaatcactgaatgtgttctcctttttggtttcattttgttcttgtgagagtatgaggatttcaaaatgttaaagatgaaaagtggcgtctagtttctgacagtttgtacagttggatgcattacatttttagatttgaagttttggttatgttagtgttatgagtgatctttgtggtggttttcttcccctggaaacctgttgctcgtggcgctttgcccacggtgcccgagttcttgtcctgtgtccagatatgcagacaaatgaagggtgaagaagaagaagaggagctttatttagtgttagaacagctcagaaggagacccacagtgagcagctcccctgtgtcggcgggcaggtcgtccctcaagtgttcagctctcagcagagaaaaggccctggagagggtgactcctctcagctctcagcagagaagcagccctggagaaggtagcttctgttcgcaggcagattgtccagaggtcctgctgctctcagacggggccctggagaggatagcttctatccataggcaggttgttctgccgtctctacaggtctctgaagctcttagcagagagggtagctcctccctgttgctggtcgtcccaccctctgctcagttctggctgagcctggggcattttacgggcctcgggggaggaagtgcatacttactggcctggaaaaggcaccagttcccactcctacaggtgggactggcagcctggccctcagccttcaggccctccctgttcatggcttccaggcttacccccctgctttgatctgagagctggtgccaatagcagggagaagccaagctgcagaggcaagcacttccgagcctgcaaaagcaggcccccaaaagtgcagggatgcctgagtctgcacccgcacccaggagggtggagatcttgcctgctccaaggctgcagccggaatgatagcaggctgactggagcacctgccaccatcattagttcaagagtttatgcagatttaagttgtatacggtatatgaatgtgtgacagttttccttatggttgtgtggccttctgtaagagcctacgcctgtttgttacaccggtagagtgctgtggaatgtaaactttccctatgtcacttatctcctttatctctccatacagaggagggcaagaaaccttgttacttgaactttagtaatgttaagtgatcaataaatctataaataaatgatagcagaaaaaagttacctgtttttgtgatgatgtacaaactttacatgttatcacaaataccatctttcttcccaagacatttacttctgtaaccaaagtgggacaccatctaacagttctgttttgggagagagtaataaccagtgcttgtgaggcttgttagatgttggttgtgatatatgagatagatgttatttcatttagacctcaacattcctgtgcgtgagatacttttatcacatcttacagataaggagactgtactcattcagttgtggagctgagattgagtagagtggctattacagcagttgagtgctgagcttatcaatatatgttccactcctcaggcttcatttaaagtaggatgcccaaacagcaccactgccgtagagatttgagttaacagcagtacttactgaggtttaaggctggcagccagtgtccttgcagtaaaattatttgctagggactcagtacttcataatctatttgtcagatttactcctaagcttctgtgttgttttattttttttctgacaaaagtagtgcatattgtcaaggaaaaactaggaaaataccaaaaaaaaagatttttgaccatgcattttaatacttagtgactacaaacattttcctattttatgcatatagattttaaataaacgtgagatcctattgtatctgttttaatggataaacattgtttcactgttttaagattctgaggtgatttatactgtcttgccattgttaattgcagcagttagccttgttgataaatttttgcatggatccaagttttgttttccaggagtggagttgcttggtcaaaggaaatgcacatttaaggttttttggtgattgcatgactgacttccctgggccctcgccaacactaggtagtagtattgggaggaaggggggaaccaatcctgggtgctccaagattactagtgagcctgaacattttctataactattgtccacttgagttgttgttttgttttttttttggtggaggcgggggtgggtttaagaattgcttatcctttgcttgtactaattatcttttcaacaaatatttctagattactgctaaggaccaagcactgttatcagcctgagataaggcagcacactagaaggaaatccttgctccttttgagtttgccttccaaacatggagatcaatatataatgttaggtagtaataggagatacatgcagttgattcatgtcatttgtagtagttatggtcaataaagttgccttgaacactgaattagtataaactgaaatactgttcctaggggaaataggttcctgctagcctgtggtcatgagatttttgtcaaacaatcactatataaccttttctgtttctgtttaaagacatgttatttgatctatatggttgattctttacattaacatggccaacagcactgtaactcagcctgaacgaagcttatctgacacatggtgttctccataaggcacatcatagctttctgtgcttaggaacactagacggcacttcagcactgcacttgaggacgttttaaacagtgaaatcaacaaaaagcacaaaaaaatgcaacaataggctgggcaaggtggctcacgcctgtaatcccatcacttagggaggccgaggcgggcggatcacgaggtcaggagatcaagaccatcctggctaacacggtgaaaccccgtctctactaaaaatacaaagaattagccgggcgaggtggcaggcgcctgtagtcccagctactcgggaggctgaggcaagagaatggtgtgaacctgggaggcggagcttgaagtgagccgagattgcgccactgcactccagcctgggcgacagagcgagactgcgtctcaaaaaaaaaaaaaaaggaacaataacaaagacactagtcccccaaaaatacacttgtttacagtgtgaactgaaagaggaaggtggagtattgacttgtttgacctcagctggaaatgtgcacgtcctgtgactcaaatttttctctgttctgtgcatgcatgtccacgaataaccacaagaagcactgaaagcattgatttttagggttacaaattaattttagcaagtaaatgaattcacaaatacggaatctgtgagtaatgaggactgattctttttttttttggagatggagtttcactcttgtagcctaggctggagtgcaatggcatgatctcggctcactgcaacctccgcctcccgggttcagcctccacctcccgggttcaagcgattctcctgcctcagcctcccgaatagctgggattacaggcttgcaccaccatgcccggctaatttttgtatttttagtacagacggggtttcaccatgttggccaggctagcctcgaactcctgacctcaggcaatccacccacctcagcctctcaaagtgctgggattacaggcgtgagccaccgcgcccggccgaggactgattcttatgtcagatggcactaaatgctatggagaagaggagtggatgagagggagaagtattttagaccaggtagacttggaaggtttcttggaggtgggtgatgtttgagaagaggcttcaataaagttagggagctcgccatgtgattgcaggaagagcgttccaggagaacaaaagtcatgaagagtgagtgctaggcatgtgtctggtctgtttgggctgctataacaaaataccttagactgggtaaaatgtataaataatagaagtgtattgcttatagttctagaagctgggaagtccaagatcaaggtatcagcacattctggtgaaagctgctctgcttcatggctggttctctcactgtcctcacatggcataagaggggcacagagccctcaaccgtctctccagtggccccatctcttagtactgttggattggggatttagacttcactaattttggggggacacaaacattgagaccacagcagcatgactgaggataagcaagaggccagtgtggttgagcagagtgatcagtgaaggagagttaggacatgagtaaagaggctagcagacaccagatctcatatggctttgtaggccatagtgaggactttgtttaagctgagaataatagataacctcaggaaagtttcaggcaagagggtaacatgatctgatctgggttttaaaaggatcactgaagtggggagactgtctacagatggtctgaataggagtcctagtctattacaatctccttggagtttagggtggtaactggaggtgttcaagagtagttggattactgttggatttcaaaagtagagccaacacgatatgtgcattggctgtgaggtagaagaggagtcaaaatgaactccaggttttattgactgagcaattgtgccatttcctgagatgggtcagatttgggaaggaaagaatttaaaggggataagataatcccattaggagtgtgttaagtgtgagattcctattagactttcgagtggagatgatttaataggaagatagatctgcaacactggagctcagcggagagggacaccctggagatagccgtttgggaattaggaatgtgtggatcatgttataggatggggtcatttagggacttaaaacagctctgaagaacaaaaatggtgccttgatcttggacttcctggtttatagaactgtgagcaatatatatatatttttttcaagacagagtcttgctccgtcatccaggctggagtgcagtcgcaccatctcggctcactgcaacctccacttcctggttcaagcaattctggtgcctaagcctcccaagtggttgggactataggtgtatgacaccatgcccgactaatttttgtatttttttgtagagacagggttttgccatgttggccaggctggtctcaaactcctgacctcaagtgatctgcctgccttggcctcccaaagtgcttggattataggcgtgagccaccatgcccagactaaatttctaacatttataaattatccagtctaagatattttgtgatagcagcccaagcagaccaaggcaaaggccaagcacacttgctcctcctgacttttgctcttcctggaatgttcttcctttagtcacatggttgcctgcctagcttcattcaataggagtgtggtgccctgaaaatacaaggaagaatgcttttcttttttttaaaaggaagggatgattatctgtcagatgctgctgaaaaagagtaatagagtaattggccactggctctggcaatagggaagttagctctgctaactccacatgaacagtttcacatgaacaagtgtgagtgggctcaagagaagggatggtgagaaagtggagctatggactcactcttgaaacattttctggtgcctcgtagggcaatgtgaggtcaaggtttttgttactgttctgaagatgggagaggctgacacatggatgttgtaggtgagagaaggggcgcttgcgggggcaaacttctccagggatgggattccagtgtctaagaggaggcggtgtgaccctaagagctagaaaaattattttattaataggaaagacaaagtacttaggctcagatgctaagagatttgctgataaaagaatgagaacggtctcttctgattattttcttggggaaataaatagatcatcagctgagggtgtgaggggagaaggagttgaacatggaggaagacaggtgtgaaatattggtctcagaatggagagcgaattgaatagggacatgcagtgggcttgctaagctgtgcggagagcccgtgggaagtttatggtcatcaatttaatggcgaccagccaagatggtggtttatttttctccagttgtatttaactgctcaggtgcaggacagagagactaagtgtgaagttaatttcagccaacgtagaggaattgtcaggcagatgggacaaggagatagaggagaaaaggaataaggcttcctgcaagggtaatgattgtagggatggataagtaaggaacacaggaagtggctgtctgctgagtggtggcagagctcagtgggtcagagcaaggttcaaagaatggcagagaggcacttgtggaggaagtaagctggctagaaagtagtgtgcttgaaattaagcttctggagatagcaaggttacaggtgatgacaaagtctgagtatgacaaggaaactgcagggccagagttggcaagaattcatgaaaaatgaggagaaagaggcaccaagaggctgggatagcacatggattgtctctgtgtgaggcaaagtcatctaaatggcagcagtggccctagcagaaagaaatatacagtgagccggagcaaaaatcctcaaggacaggcagaacgccatgaaaacggcagatgacagccaaaggagcaggggcaggggctcagtccaaagtgtttcagagtcactggagggttgagtgggaaggggagggagtggctgaaatggcaacaaggaagaacctctctcatctccaggcccaaaagtatgtggaatgcgggagataagacagccaccactggccagggctgtaaagggacattcagcgaatattcaggttccatttagcacgacagcagggaagggactgttggcagaaaaaaactggggcagtgggattaaagacagaccacacattccaaaaggcaccgtgggagggtcagggggcgaggttaggtctaggcttcagtgtcctgggagactcagtcttcacagggtgacagcgatcaagagtgcagcttaggctgggtgcagtggctcatgcctgtagtcccagcactttgggaggccgagacgggaggattgcttgaagccaggagtttgagaccagtctgaccaacatggcaaaaccccatctctactaaaaatacaaaaatcaactgggcatggtggcgtgtgcctgtagtcccagctacttgagaggctgaggcaagagaatcacttgaacctgggaagcagaggttgcagtgagctgagatcgtgccactgcactccaacctgggcaacagagtgagaccctgtctcaaaaacaacaacaacaaaaaagaaaagagtacaacttatgaaggggtctcctggggagagggtttttgggattctcctgcctctcaaagtgctgggattatgggcgtgagccaccacacccagccgagggaggctgagttctaattgttgtatctctcttgggattggcctcctgggcagtttaaaagacaaggcaaggaatcttttggagaaagagactgggggcaaggtgtgtctgaacaagaagtgtgagaagctctgtgggctcccttcagacttccagtcgttgaattgggatctcatttatatcagctctaggtgtaacgatattaaatcttctctgtcatttggcaattttggtttatgcttgatcatcatttttaatgtttcgacatgtagaagtttaacattattttacattcttttccttctggcatcatgttttagcaagattgtttccaccaaaagaatatatatatcttctaatgaaactacgtttcttttttttttttcctttgctttctcttttggtatatgaatctttgattatttgtaatgtattttgatgtgtaacactgaagtttctattttgtactatttttttccccaaacagtaaacttattgttcaaatacttattgaacaaccttcactattctttaaccatttagaatacgccattcacatatctttcatactacatttaataacattttttaattaaaaaatattctactgatttgtttattttgagaccaggttatgaaactggctaatttttgtatttttgttaaataccgaaattcactgtgttgccaaggctggtctcgaactcctgggctcaagcaatctgcccaccttggcgtctcaaagtgctgggattacaggtgtgagccgctacacccggccacacccggccaacacatattatttgttattacatttaattcccacagtacattgaaattatcagggaaaagttttcagtgaaacattattgaacgccacattaaaagtgtaaattacaaagatttaatgccaatttttcagaagaaaaaagaccaggaggaaggtctatgaagttttagccagtctctcatccacctaccatttcacgatcatgcactgtgtaagtcaggaaaagagtaagaaaagtgaaagatacaattgattagagagttttgctggatactatagatgaaaagaacacaaaatggaacagcctcttcaagcttagagtcaacggctgtagtcccaaagactgtagtcagaggcggtagggccaaaagacatgacttatggcattggaggaagaggatgctttgggagttcatggtagaagaggcggaaaaaatctggtggattaaagaaagcatcccaaagtgacattaaactaatgactaaattctgagctgttttcaggggcaaagcctgtttgggcacccctgccacacttaaagagtcacctaggtatggttcgtgggctctgaacaggcctgctcagtgaacatatttgtgactgtttctccggcccttttagctgtattgagtaaaatttaaagagaccattgttttggcctaagctcctgccctaggcccaaagaacagaccaaacctgaatggcttcacttgtcctaggtgctgtgtactcaaactgaactttgaaacaggtcggtttttcaaaaaaagcaaaagattcacagcaaccaattagaagaggcccggtcaacctgagccagcatgatgaggctcttctgctttaatcctacaaggaaagaaactttgaaatgaccaatctgctttcattcttggtttctgctttctttggtctatttctgcctgtaaaacctatctcctctgctcagctcattgaagtacccttctatttatagatgggatgctgcccgactcatgtatcgctagtaaaagccaattaaattattacactcgatttgttggaattttgctattttgacagcttttcaaaaacaccagtaggttcacatccctaattccccagccagtgttccctcaaggaaccatggaagaagcaaaggtggctgaaaggcgcctcaggatgcttctaagcacggcacatccatgaaaaggcacttactaatatttgcaggatagcaaagcactgcagtgacgataaatctagtattggagaagttcaaaataatcagtagattaacacagaagccagagcttatagggagaaaaggaaccctatgaaatacttcaaatccgaaaacgaacatgcatttcctgtttagttagtgcaggtacgtaaaagcttggtaaagtacccttcttgccagctttctctttcttacaagccttttcactgggctgggaggctgatattatctaaatatgctgaggaggttcaagtatctccacaactcacctcagagtgaatgctcccctcggccttaaggcaatataaaccagccctgtttagcaggatagcaaaatgtttgcggttgtaaactggtgtcccattggctgtggcgcttgtggtgtaaagaatccctgtgcttggtaattaatagagaaattctatattttaaacttcagttgtatattggctcttatccatggcagattttcacgtatgtgttatttttttatttattcagagccggagtctcgctttgtcgcccaggctggagtgcagtggcgcgatcttggctcattgcagcctctgcctcttgggctcaagcaattcttctgcctcagcctccctagtagctgggactacaggtgcatgccaccacgcccggctaattttttgtattttagtagagatggggtttcaccgtgttgctcaggctggtcttgaatttctgagctcaggcaatccgcccgcctcggcctcccaaagtgctgggattataggtgtgagccatcatgctcggccctatgtgatatttattacaatgaattccaatgatcagacctatactcaagtataagtgaatatatcattcaatgaagtataaatgatcattatgttcatattcacacatacaataatgtactcaagtttattgctaaggtaattcagaatctccttattttgaagtgtgcatttgatatacctgtttgggaataactagtttcttatctttgacagaaaataattttgttgttttgtttttactaaaaaagcatggtgaaaaatggctccatttctaagagaggtaactaaaatatcgcaatttgctgggtgtcattaaagtaactcacaagggaaaaaatgcaaattggtatctgctgatggagtaaatctccgcagaagtgatgaccctgaaaggatcaatatattaaagcccctcccagctggtcattccagattgcaacaataaagcattaagtgttaaaacctcaaggcagctttttttttttttttttgtctcaagtcctttattattaattttatagacctacttaattactaagccaaaaaaaatcaaacttgtttctctttgtgacttgtcaatagtattaaactattctggttttttatttttgtgttaccttaaagtctccagtttagtaatttttctgtacctaaacacttcggatttgacatgctttgtggcctttatcagtagttagaatgtaaatccaataaataaagtaaaagccaggtcttcaaaacctgggggccaagaactctgttttagagggcctgtgactctcttggacactggacaaaatctcatctctaaatatggatattttagggagagggtctttaggctgtcatttggattttcacagggctccatgtatccataaggtagtctcttgggaagtttgacttcaataaatgaagtttaacttaaacctaaaatgaaatttaactgaaaaacaaaatccaatgaaagatgctttcttatgcaaaaacaaacaaacaaaaaaaaaacaaaaaaaccccaaaaaacccaaagccaaagattgtttctgaaattaggttctaggttccagagcaactccatggtggggaatcagccacatgtaaagtaagctaagagtttggacaatttgtaatatttattcctaggtttctttaagaccctttcagattttgaattcctattagtagcatcagccaggttctaaatgtaggcatcaccatagacacttccccactgctgcagtccccaacacttgcccaattttcccttgaattgcacccatgctgccttctccaggcctatttgaacccagaacctcgttgtgcctcgtttgaaatataatttcctcctaactagtctctgatctactatttcccctacattgctgccacactaatcacctaaaatagatttcattctaccctgaaacagaaatctctaataagttactcccttcccttacggggtaaagttagccacatcctaggtattcaaggaccttccaggagctaagaacatttcccctgcaccttcttgaagtacacttgtcctatgtactggttatgttcatttcttaccctcgctctcgttttgtctggaattttccttggccttaaatgcctctcacctgcctgcccacatctctcagggttgtttcaaatcctcaatgaaggctcacagccccagtctatgttggccacttacttcgtggcctgggaacatttttctttggctgacttgctgacactccatcagatgcatttttatctggttgtccatctgtgaaccataccctgagaaggcagagagtgcctctgcactgaacatgtgctaggggacaggtctgtgctagaggggcaagcactgggaatgaagaactggtccctactcccaaggagttcatatctcagtggaggtgacaagcaactcactgtttccgggggttgtggtgactgctgggagaaggggtgtctatattagatcgaagcagcatcaggggaggttccctgagaaggtgatgcctcagcggatgtctcccagctaagtggggtggaggtggagaagggcagagcagggagaggatctaggtggggcgtgtaagtctgcatgggtaactcagggaacccttggtaactgcatgtaactgtgtgaagctttcatgaaggaacatggtaggagactagggtatggactatagaagcccttttgctaagctcaagaatttgaggccgggagcggtggctcacgcctgaaatcccagcactttgggaggccaaggcgggcggatcacgaggtcaggagatcgagaccatcctggctaacatggtgaaaccccgtctctactaaaaaaaaagtacaaaaaattagcggggcgtggtggcgggcgcccgtagtcccagctactcagggagctgaggcaggagaatggcatgaacccgggaggcggagcttgcagtgggcggagactgtgccactgcactccagcctgggcaacagtgcaagactccatctgaaaacaacaacaacaacaaaaaatttgaagtgtatcttgaaggaaatcccttggagcctaaaaatgatcattgataacagaaaatgatctctgctctcgcctagggtaatatattcagcttcaaagtggaagggcatgttttccaagggcatgttttctaagtccctgtaattgtagtgatagcaaatatatgccctgcatcttgaaatgtaagactaggtttgaacagtatataaattatcttatgatctaatttcccctcattttgtggtttctactataagctacccagaagtgtagacaggacgtttggaatttgatgggcatcggaaagattcctacctaagaacatttttttttttttttttttttctgagaaggagccttgctctgtcacccaggctggagtgcagtggcacgatctcagcttactgcaacctccacctctcaggttcaagtgattctcctgcctcagcctcctgagtagctgggactacaggtgtgcaccatcatgcctagttaatttttatatttttaataaaggcaggatttcactatgttagccaggctggtcttgaactcctgaccccatgatctgcccaccttggcctcccaaagtgctgggattacaggtgtgagccactgcgcccggcctctaagaaaatttttgagagctacttgttctgttgcctggaattccaccgtaagtacgacgttgtgtctccttctccagggctactaactaaacaacagagggtattgtgttatcgacaattatttgattgataactatcagcaaacatttgccaaggcattcctttaaagatagcctagtgactctattaactactccttcttccaggcttctaagttctgttggaggtaagtagatcccagagataaagcacctaccataggacctgaatcttggtagaaataaattatatcatcatgttatcatattatcatgtgtttttctatctttaaagtcttatgtgaatattctgcttgaaaaatatgtgtcctctgttagaccagagttgaaaatatgttattcaagaacttgtaacaggaacccgcacaatttctgctggagtttaatttcagggttaattctgtcagcaatctaaggtaaacattaacatttttccctagattcaagtccgttgtccaaaagctgtaacagaacttaactgaataaatagtttcttaagatggtaagcttccatatgcttataatgactcctctacacgttttcatctggaaggctgctcatgcttttggaagcaaagaagacaatcttaaataactacatttgctttttggtggtgccagatttttctgagaaacaccaatggaatttataaattcaccagtcaatgggcaattgagttgctgttttgctattaccactgccgtttgtgagcattgttgggaaggtgtcttgaagcacacgtgcaagtttcccttggataagtagtaggaatagaattgccaaaccatggcttccagtgcagacacagtctctcccttgggcccagccactaggcaccacacattaagaggatattgtctgtccatgtcctagaaacgttgtagcatcatgctcctattcgattaaaaatctcattattaaaatgaaccatcgggtaaatgttgtctcgggaaaagaagcactgaccgtccctgggtgggctcgaaccaccaacctttcggttaacagccgaacgcgctaaccgattgcgccacagagacccagttactcaggccgcgctgcggtgtgtacagatttccgcggcgccggcagccgctctagccaccctgggcgtcgccaccccaggcgttgccaccccaggcacgggctgagaagtcgcggggcgcgccgaggaggcagcggaagcggccgaggtgcccagcggccgccgcggggggagaggctgtgccccggcgcgcgggagggggcgggcgaggccgcgtgactccgggcttctctggggacgaagcgcgcccctcgtggcggcagcggccagtggtccgcagtcggcccggactcggggtaggaaagatcctctcagcaatggctgcgcgccatgcgtgctctgcggcggggaccgtgccggccgggcgcgccaccagtaaccagggacccaggggagaacctgccaaggggaataggtcgcacggagagaatacgacacgcttggagggaagaaccacgtgctgtacaggtttaaaggatggagagtcacgtgcgcttaggtcccaaacttaagggacctaaccctttttctgggttgccgctattgccccttctccttagacagtttttcatctcatcacctctcaccccgtaaaatgcaacgaacatagataggctgtgtatcaatgtagactgtatgtatatctgtgcttcgtacataaaaagaatatgatttttgccaccttctaagaaccaatttgcaccccattttgaggcatatggcctctgttgagattgcatagtttaggggacatcaaaaaagccttatagagggactggcaattaagatagcctttcagtttgaaatggccattgaaggcttctccctttccctgacttctgaattttttttttttttttttttttttttttttgagatggagtcttgccctgttgctggagtgcaatggcgcgatctcggctcactgcaacctccgcctcccgggttcaagcgattcctgcctcagcctcccgagtagctgggaatacaggcgcctgccaccacgcccagctaacttttgtatttttagtagaggcggggtttcgccatgctggccaggctggtctggtactcctgacctcgtgatccgcccgcctccgcctcccaaagtgctgggatgacattacaggcgtgagccaccgtgcccggccaatttttttaggcgcactgttcagtggcactaagtacattcacattgttatgcaactatcaccgccatccatttccagaaccttttcatcttccgaaacagaagctccctacccattacacggtaactcacgattcccctcctctagtcggaacaatcaccattctactttctgtccctttgaatttgactactcttagagacctcatgtaaatggagtcatacggtgtttgcctgtggctggcttatttcacttaccatatgtcttcaaggtccatccacgttgtagcctgtgtcaggatttccttcctggataaggctgaataagctgcactgtatgcaggtatcgcattttgcttttccattcatctctccgtgaacattagggttgcttccacctgcagctatgaacatgggtctacaaataactgattccctgctttcaattcttttgggaatatacccagagatggagtagctggatcacatggtttgctattggctgtaccattttacattcgcaccaacagtgtacaagagtccctatttctcctcatctattttttttttaaataatgggcatcctaatgggtatgaagtatcatctcattgtggttttgctctgcatttctctaacgattagtggtgttgggcatcttttccagacaccaccaatctgaattctatggcccttcgtttactcacttcctcccagcaagagccatttctgcttcagcaaggaggaagctgcgactgatagagggaaagggcccagggggcttgcagagtggggcctgtgccatgcaaggagaggagaagaaggtggatctttgagtaggactatctggagatcctgctttcacaaggtccttgcttgtgtgctgggcagcttttggagctagttatctttattttagcccttgagggatatttaggcatgtggtgcttgtgagcagccaatccatgaagaaggaactgatggtctccaccttggaaatattggaagagataatgccgtccaaattgcagttttagaagttaacttaaaattatgctattttaatggaattttgggtgcatttccattttcttcttaagaattgctggaatttcttaagtgtttaggtgatgatctctttttgtgattccttttttaaaaaacaacaacaaaatctttcaaatacataagaaataggccgggcacggtggcgtaatcccaccactttgggaggccgaggagggcggatcatgaggtcaggagatcaagaccatcccggctaacacggtgaaaccccgtctctactaaaaaatacaaaaaattagccgggcgtggtggcgggcgcctgtagtcccagctactcgggaggctgaggcaggagaatggcatgaacccgggaggcgaagcttgcagtgagcctagatcgcaccactgtactttagcctgggcgatggagcaagactgtctcaaaaaaaaaaaaaagaaaaaaaaagaaagaaatagacctttatttttctgtaactccacaaaatttctattttgattccctattattttgctattgtcaacacagtctcagtcaattcaagatcctgtttgtgcctttccctggagtcatttccaagtgctaaggctttggtccatgagtcgcatgtgcacactcatggctgtagagggagttttgctcccggtgaaggtcttggtggctcttctataccttgattgagggaaaggaatcttatgtgaagttagctttgttgtatcagatattccataaagccatttctgggacagtcccctctgtttatcggaccacaagcttctctgtcctcatcaagcccacctttatacttcatttctccagacttcatgtccagactgtgggatgaacaagtggttataaggttttagaggctcctgtaggactagatggaaggcaaaaaaaggaaataacctttaagcatgctctcgattccttaaatcccatctgaaagtcttaaggatgtcttctcagtcatacttatttgacaatattacctaattttctccattagcccaagctcaggggtctttcttcttccatattcacatgggtgcaatggttttctgaaaggaaaacagcattactagggcagtaacatttaattaatcacaggtacttatcaaactacaaaacaggcattccaggaactgggtgtttctgtttgtaaaattacactctcgtgtacatgctcccactaaaatgtaagttcgctgaggatggaggttttggtctctttgctctgtgctgtaaccccaacactgcagcagggcctggcacatagcaggcatgcagggactatgcactgaatcaatgaggaaatgaaaaccaggaccatgaagtaaactggacaaaataaaatgtgatagaaaatctaaattcctaatacataaggagcacttatcaattgatatttacaaaatctttttacaattcaattaaagacaacataaaacaaataagaatggggacaggaacagaaaattcccccaaagaaaaaaatatatatacatggtacagccattgtggaaagcagtatggagttctcaaaaatattaaaatagaactatcatataatccagcaatcccatccctgggtatatatctaaaggaaatgaaatcagtaccccaaagaggtgtctgcactcccatgtttattgcagcattagttacaacagccaagatatggaatcaacccatcagcagatgaaaggataaaggacatgtgatacatatacacaatggagtagtattcagccttaaaaaagaagaaaatcctgtcatttgcaacaacatggatgagcctagagaacatactaaatgaaataagccaggcatagaaagacaaatgctgcatagtctcacttaggtgtggaatctaaaaaagtcaaattaaaaaaaaatgtcaagcagagaatagaatggtagttgccagggactctgggaagtagcaggggtgggggtggaggggaggggatgggcagaagttggtcaaaaggtacaaagtttcaggtagacaggtgtaagttctggggatctattgtacagcgtggtgactgtagttaatactgtattgtgtacttaaaaattgctcaccaaaaatgttctcaccaaaaaaatgatgtttggatatgttaaacagtttgatttaatcattttgacgtgtgtgtgtgtgtgtgtgtgtgtgtgtgtgtgtatacatcaaaacatcacattatataccatatacaattaatatatacaatttttgtcaaagaaaaaatgcacatgaccaatatgataaaagtttagtctcactagtaataaaaatcaaaattaaatgaaataaaaatttctttccccaaatcgcaaaagagaaagaaaggtaatactaaaacacagtcacggtgtagtgagagggctgctctcacacaggactgatgagaataaaattggagagcagtgtggtaatatacatattaaacaatgtatataccctctcattttagaaattctatattagaaatccatcctaagaaaataaccagggatgtgatcaaaattttgaatgcagcagcacagtattatttataatagttataaataagaaacaacctgaatgtccagcaacaggcaaaaatgataaataaattgtggcatatttaagctggtggctcatgcctgtaatcccagcactttgggaggctgaggcaggaggatctcttgaggccaggagtttgaaacctgtctgggcaacataacgagacccagtctctacaacatattttttaaaattaggtggggcatggtaactcatgcctgtaatcccagcactttgggaggctgaggtgagcagatcacctgaggtgaggagtttgaaactagcctggccaacatggtgtaacaccatctctacaaaaaatacaaaaattagccagggtggggtgcgttcctgtagtcccagctactcggcagactgaggtaggagaatcacttgaacccgggattcggaggttgcattgagctgatatcatgccactgcactccagcctgggtgagaccctgtctcaaaaaaaaaaaaaaaagaaaaagaaaaaattagctgggcgtggtgctgtacgcctgtagtcccagctattccggaagctgaagcggggggattgcttgagcccaggaatttaaggctgcagtgagctatgattgtgccactccgctccagcctgagtgagaaagcaagactctgtctcttaaaaaaaaaaaagtgatatatttttaaaatagagtatattacttatatagacatcaaaaacaatattttcaagggatatttaaaaacataggatcatgacaaaatgtaaagttcaaaggtaagatggagaatggagaactgtggggaactgtataatctgacaattcgtagttgcatacatctttctgtgtgctggtgctgttagaacactttgtacgcatcacctcatttaagttcagcatccctaggtggcagatactattattatattccagttttgtttcacgttgtatatgcggtgtgagccccaatatgggatgtgtgtgtgcacatgtgcagtatttggaaagttctatgaaatattattagtggttatctctgggaggtgatttttattccttttccagtatgttctcaagcatttgctgcaagcagtcttttgcggggccagggttgagaggcagcagcagtttccctaaattacagatagagggaggtaggtggttatgcttggccagatctctgtctaggggtagaggagtgcctgtgtgtgggtagggacaccggcggggggctttgccaaacacagtggaactgtcacgctggtctctcttctcaactctttcactcacctgagaaaagggtgtctatggaccatgcacacttctgtggggaattttacaagatgtgaatcatcagtgatgaagatgctttcatttaaaaagaattggagtacctgagattagagataacttctacccttttaaaatatttttaaaaatttctttgcactgattttttttcttcgtttttatgagttgttttcatttgggtgggataactcaatctacaggagaatattaagactttttaaattttaaaaaatatactttcaaatacttaatacattttgtgttaaatgacagccagcagatattgactgaattgggctagatgcttcagggatctcccttccatttaagactctccgagaggccattcctgactgcaggtcactgtattatttttaattttaaaatttttacttacttattttatttaattttattttttgagacagagtctcactctgtcgcccaggttggagtgcagtggcacaatctcagctcactgcaacctccacctcccgggctcaagcgattctcctgcctcagcctcctgactagctggggttacaggtgcaggccaccacaccccgttaatttttgtatatttagtggagtcagggattcgccatgttggccaggctagtctcaaactcctgacctcaagcgatccttccacctcagcctcccaaaatgctgggattacaggcctgagccaccccactcggcctactttattaatccacttgcagaaacaggatatacacaaaaacgtttcaaggctgtaagtgccactgcatggcaccaatggtaaacgttttacaaatttgagtcaggaacaatcattagtgtcactagcaacaaaaatcaaaattaaatgaaataaaaaatttctttccccaaatggcaaaggagaaagaaaggtaatactaacacgcagtcagggtgtagtgagagggccgctctcacacaggactggtaagtacagagccatggagtaagcaggtcttgagctgacactggagaggatccttttttttttttatttttatttttttagagtcagggtcttgcttttttacccaggctggagtacagtggtgccatcatagctcactgcagcttcaaactcctgggctcaagagatcctcctgcctcagcatccccagtagcagggaccacaagtgagaggatcctttagtgttgtcaaggagaaggaacagaggtgtggatgggtgggcacagacacaggagcacagctgaagcagaggattacaaagggtggagcctgatgtaaagaaacctaataggtgacagagcatggaggctcttgaataccaggctggaaactgcattaggaacggtgctcataattgcagaaaattttacatggcctagatagtcatcaaaggatgatgtacaaacaactatggcatatttatacaatgtgccgacaggatgcactgaacattttgaacaacaaagagacttgataatggcgaggttttgaggaggtgaatcaggatgcaaaaaaagcaaacaactaataaagttgattgatgacaaacactatcaaaaggcagccaggagaaaagctactggttacctccagggagctggtgagggaggctgggtgggaggatctacccttctgaattctgagggcacctccagtgtggccctcagaaagcaggagcttccaggctagaatcagatcccgacatccctgttaattccacggattccacaccgagtcagatttatgatttactatagggttttaaaaaccaaattgcagggatgctagcctatcacagcttatctcagacattgtccactaaggtatacagagtgctgcctgttcctttggtaccctaatcaggaaaccccatcagatctgctccttcctatggggtagtgagtaacacgaaggcttaccatctcacacagataactggtcataggtccagcagaagtttaaaacagaaaatgaggaaagccatgtgattaactgctgccagactgtttgtgttacaaacagcagttccttaggcattgcctgggacatgcaataatttctgttacacaatctgtggtagttaaaatgctgcacgatgaaagctatctgatttggattcattattaggtgagccatctcgtctgcaatttggttccaccattttcatttaacaaatgtaaaaaagtttattaagctcttacaaagttatgctgggcaaatatgcaaaagtccagatcacctaccgcaggaactaatctagcctcctctctgggcaccctgttgtttggggctgggcagttctttcctgtgtagaaccatctagggctgaataggtcattctgacacctgggcacctctgcctgctcgtaaatgggacaatcagaaagggcccttatgtttccaaactttctttaaagtagctgttctgaaaacatggtccagggacccctgattgtccctgagacctttgaggggatcttcaaggttaaaattaatgtcataataatactaatatgttatctgtcttttttcactctcactttctcacacgtgaacagtggcattttccaggtgacagagtgtgtgataatgaacctaactgaatgcagaagcaaacatgagaacctagttttttcaatcaaaccagacgtgaaagagatttgcaaaaatgaaaaaacaatgctatcctcctcacaatatttttgttttagaaaataaagttatttttcctagaaatgtttttgagtttatcagtcataggtttattattataattaaaaaatgaaatatacatacacagacatattttttaaagttctcagttttaatctctttttttttttttttttttttgagacggagtctcgctctgtcgcccaggttggagtgcagtggtgcgatctcagctcactgcaagctccgcctccctggttcgcgccattctcctgcctcagcctcccgagtagctgggactacaggcacccgccaccgcgcccggctaattttttgtatttttagtagagacggtgtttcaccatgttagccaggatggtctcgatctcctgacctcgtgatctgcccacctcggcctcccaaagtgctgggattacaggcgtgaaccaccacgcccggtctcagttttaatttctaatacagtaagtattgatcagtgtgccccacattagtaaaagctcttggggtcctcagtacttctttttaagagttgtcaaggagtcctgtgaccaaaaataggagagccactgccctagaaggacagccccagcccgggtcaggaacaactgggacagaacctactgctcctagtggattgtaatatgataggatttaaccttcaaggtttcaactcttggcaagagtccatgaggggccatggtttgtcctgagcattgcttactgttaacaggagcaagttccttaggctggtgagccaagccagcctgacgctggccatggacatcttagtgggctgcttgttctagtgtgggttttcattttatgggaaatgtcatctgctctaaggctcttctcatttggggaaatcacaagttctcagaatgtttgtctctcttggttggggcctctataattaaattataaaacagaggtaatggttaagtaatgcaagatttgacagaaaccacagaggatttagggtttaatttgagtgaggcaaaggggggatgaagatgagcggtcctggagacaagaaaaagattggatgaagctgggcacggtggctcacgcctgtaatcccagtactttgggaggccaaggtgggcagatcacttgaggccaggagtttgagaccagcctggctaacataatgcaaccccgtctctactaaaaatacaaaaattagccaggcgtgttggtgtgtgcctgtagtcacagctacttgggaggctgaggcatgagaatcgcttgaatccgggaggcagaggttgcagtgagcagagatcatgccactgcactccagcctaggcaacagggtgagactctgtcttctttttttttgagacggagtctgtcgcccaggctggagtgcagtggcatgatctctgctcactgcaagctccgcctcccagcttcaagcgagtctcctgcctcagcctcccgagtagctgggattacaggcatgtgccaccacacccagctaatttttatatttttagtagagacggggtttcaccatgttggtcaggctggtctcaaactcctgacctcgtgatctgcccgccgcggcctcccaaagtgctgggattacaggtgtgagccaccatacctggctgagactctgtctttaaaaaaaaaagagagagagggagagaaagattggatgaaacaacagagtggggaggacctgtgagcttggtagcttggtgaaggcagggctttattgggggccttagaggggatccaataaaggttcccagtcatggtagtgacctaaagaaaatagcattttaacatctttcatttcataatagacagtcacagtttacaagaccctttccatacattccttatgacatccatactacagcccagaggcaagttgtgcactctctcctctcacaaatacaaaaactcagcctctagaggccagcgacctgctcagggtgatgtgcaattcagggatgacagagtcgaggctcccagcccagtggttatccctcacaggcacgttgcctgtcagtgtgcagtataaaactttgtacaagaaatcaagttgcattagtcagtcggattccccaaatgatcacattgtagatggtgtatgctgtgggcagagcaagggctgctgtttcttgggcaaaacaatcagtccccctcccccccaaaataaatgaatgccaatggtgtgactttattttatttattttatttttattattatttgtgagacagagtctcactctttcacccaggctggagtgcaatggcatggtctcggctcactgcaacctctgcctcctgggttcaagcgattctcccgcctcaccctcccgagtagctgggactacaagtgcatgccactgcacccggctaatttttgtattttttttaagtagagacagggtttcactatgttggtcaggctggtcttgaactcctgacctcatgatccacctgcctcagcctcccaaagtgctgggattacaggcatgagccaccgcgcccagcaatgtgactttataattacagaatgtaggactcagctcccactattgttatgactcaatattctcttagataatgtttggggcactagcttacaggcagcattgcccggtggttaatgttgtagctttgcaggcagactgaccatattaaaattcgatcacaccatttgctaagcctgtggactcgggcacgcttctttctctgcgttagtttcctcctctgtaaaacacggatgatgctataaacacacccaagtcctagaattgttatatgagttagaaaagataggcaaatacaactctcacaagacagcctggcctccagtaagtgccactgagtgtttgctcttattgtacagtggctccaagtgcttctgtcttggattatttctgaccaggtggctatgtctcctagtaacttaccaatcctgttgagtcttaataagcacgtctttgatgcctacagtgcgactgaatttccaggcctcattactggagacacaatcatcctatatgcttttttccatttgtttttaataaagtggtacatgtgtatggcaccagatcaaacagtacagaacaagttacaatggaagagaatggcctcccagctttcctgaaatcctcaactcagagacaacttttttttttctgacggtttctttatacagccctttttgtggttaccttcctaactctagaaaaactattcttacctctgtttatttacttagaaacattagacgttacctttcaactcctcagtatgaagctttagttttcagcaccccaggccaccaccctctttccaggacttactacttatactggtggtaggtggaattttaaaattcatcagcattcttttgtgattctctgtgtgttccagttttacagcaacccgtacttgttgcatgagtacagtagaactgggaggctcataacttagcctgcaggacttttcacttaaagcctggccctcagggtgatgtcacccacctcattgtgcctggctcaggagtttagtccctcagttgcctggttgtatagtttggatgttcagcacctccaaatctcacattgaaatgtgatctccaatgttggatgtggggcctggtgggaggtgtctgggtcatcaggtgggtccctcttgaatggcttggtgccttccccatcgtaacgagtgagttcttgctctggcagttcacacaagagctggctttttaaaggagcctggcaccttccgctctttctcttgctcttcctcttcccttcctttgtcactaaaagcttcctgagccctcaccagaagcggtgcagatgctggtgccatgcttggacctcctgtagaactgtgagccaaataaactctttcctataaattacccagtttcaggtattcctttatacaatgcaaaacagactcacacatctggtaaaccccagttgtttgcttctaggtaagacgggaggagtggggagctggtgagggtttccactgcattgtctattttcaggcaaggtgtctccactgagtaggcttcacattcagagctctgggtaaggtgggcaggaagagggttgcaggctgcccaaaggagggagagaagaaggctgaatccttcagtgacaacctgtgaaccagagtcttagctctctttgaatattttgttcagtatctttgggttttgttttattttgcctaggggtaaatgctgactgcctgttctctggacaggaatggagaagatggtgctagcagggttgctgttcatatgtagacattcatgcagtcactctcttttcagcacacttcttacttctgccctgggttcagttgctgactctgagcccagaaaccttctagggttctgttaggtagattggcttccaccgtctttgcgacaaccacagaaaattctagactgttttctcttcgggcttcattagtcaacttgcttcagtctgtcttgcatcttctaaatatttatagatctctctcttttgttggagtggcagaaaatgctagttgaccacccaatattcaaattatcctgcctccttaataacagaatatcattggatgtggtgggtaaataatataccctaactttccttgcagagaggggtggccaatgagatggaaatgaaagtcattgggaaagactcccaagacatctctttaaacaagacagactgaagcaagttgactaatgaagcccaaagctagcagttgtttttgtttatctttgcctctttcttcttcttcctgtggggacaaagggcagtgatatctggagctgcagcagccattttggcataatgttggaaaagccaagagactctcagagaccgcagctccagcagttttttattttttccaaatatttgctccactgcaggaggatgagatattcgtgtttgttgccttgtgactgtaggaggactgcacttccctgccttgttgtcaagtttccccatgtggtctgctttggccagtaaaacatgagtgggagaagcttggtgaaccattgcatgtctaccagcttttttgctctcttccctttggcattagaaaggcatgtccaggatggagttgttccttcagcctagattgggttatgagaagctagctgggggagtccagtaacatataaagcgagttagaaataaaactttgttgttgtaagctatatatatatatatatatatatatatatatatatatatatatataatatgtatgtaatatataaatacatattatactttaagttctagggtacatttgcacaatgtgcaggtttattacataggtatacatgtgccatgttggtttgctgcacccatcaactgctcatttacattaggtatttctcctaatgctatccctccccagccccccacccctcaacaagccctagtgtgtgatgttccccttcctgtgtccaagtgttctcattgttcaattcccacctatgagtgagaacatgtggtgtttggttttctgtccttgtgatagtttgctgagaataatggtttccagcttcattcgtgtccctgcaaaggacatgaactcatccttttttatggctgcatggtattccatggtgtatatgtgccacattttcttaatctagtctatcattgatggacatttgggttggttccaagtatttgctattgtgaatagtgccgcaataaacatatgtgtgcatgtgtctttatagtagcatgatttataattctttggatatatacccagtaatgggatcactgggttaagtggtatttcaagttctagatccttgaggagtcgccacactgtcttccacagtggttgaactaatttacactcccaccatcagtgtaaaagcattcctattcctatgtctccacatcctctccagaatctgttgtttcctgactttttaatgattgccattctaattggcctgagatggtacctcattatggttttgatttgcatttctctgatgaccagtgatgatgagcattttttcatgtgtctgttggctgcataaatgtcttcttttgagtagtgtctgttcatattgtttgcccattttttgatggggttgtttgttttttttcttgtaaatttgtttcagttctttgtagattctggatattagccctttgtcagatgggtaggttgcaaaaattatctcccattctgtaggttgcctgttcactctgatgatagtttcttttgctgtgcagaagctctttagtttaattagatcccatttatctattttggcttttgttgccattgcttttggtgttttagacatgaagtccttgcccatacctatgtcctgaatggtatcgcctaggttttcttctagggtttttatggtttttaggtctaacatttaagtctttaatccatcttgaattaatttttgtataaggtgtaaggatggtttccagtttcagctttctacatatggctggccagttttcccagcaccatttattaaatagggaatcgtttccccatttcttgagctacagatattttgagtttggttaccacagtattatctagtggaagttgacttatacagtatgtaataggataaatataggtgtgtaacagaatattaagtgttcgtgtttcaaagctgaggggaaaatgttaaaagtgttcacacactctaaaaagagattagctaaaactgcttcattaaccacactttggggaaaccagttctgagattcttctccattactctgacaggttggaccctctggggagcagatctcaagatcaagttatgagtgcaagaggtgtgttgggaagcgatggttgtaaaagaatcctgcagtagcaccaggcacaagtctgtccagggagaggaggacttctactctctaccagcatctctcctaagtccccttaggggacgggggcaaggaagtgctgggaagggcagggcatggttcctggctaggactccacccccctggggcctgtacccacggacctaggtgaagacaggcactcctgccttctcgcccaacggttgcgtttcccaagatcatcctggcctgccacgcccccatctacctattaaactcccccaccttccccaaaccctagcaggcagacacacatcggtggaagaagacaggagcggctggacattgaaaggacgtcgagaggagcacacctgcacaccatcgaccagcggaacgaggcagagtgtggctggagcagtcggagggaagcctgggccgctgactccaggggaaaaccatctcctttctggctcccccctctgctgggagatactttcactgaataaaaccttgcactcattctccaagcccacctgtgatccgattcttcctgtacaccaaggcaagaacctgggatacagaaagccctctgtccttgtgataaggtagagggtctaactgagctggttaacacaagctgcctatagacagcgaaactgaaagagcacacaatagcacacactcattggggcttcaggagctgtaaatatccacccctagacgctgccatggggcgggagccccacagcctgcccgtctagaggtttgagcagcgggacactgaagaagagagccacaccctcatcgcacgtcctgcgagggagacaagggaacttttccggtttcacttctgcttggcttgagctggcactgaagcacccttttccctcctcactgagggagcagaggggaaaagcggtagaactaacaggctaacaatgctcctccgaaaatatatcgtatttttggatccctagagataggtgatcacggcagccgcggagtgcatttgggtctcctttcaagaaagaacttgctgctcagcgttgaagaatgcagttggccaacagcctccagctgctctgtcttcagcatctgccatggcatctgagctgaggtcatgttcttcctgggaggtccccagcagaaggatcacgtggaagctccacaagctccacagatgttccaggagaggaataggcagcatttggaagacatatcctgccataacagagggcatttgctagtagagacaacaaacagcaacagccaagtaaacaaacacacaagcacaaagcactttctcccatttcccctcattgatcctgtccgggtagaagctggggaggaagtagaatagggtgaggcggggtggggctggggggcctacaccttcttccttcccccgcaggtcctgtccctgggccaggcttgaactaggggaatgggaaaagctgtgaagtgaatgagaattaggagtttttatttagactggacttgaatttttttttttttttttttttttttgagacagagcctcgctctgtcacccaggctggagtcccgtggcgccatcttggctcactacagcctctgcctcccgggttcaagcgatcctcccaccacagtctcctgagtagccgggattacaggtgcctgccaccatgcccagctatttttttttttttttgtatttttagtagagacagggcgtcaccgtgttggccaggctggtctcgaactcctggcctcaagtgatctgtccgcctcggcctccccaagtgctaggattataggagtgagccaccacgcctggcctggacttgaatttttaattcctaaaaatgaactaccagttaaaatttaaaaatgaccaaaaaagctatgggatatgctgatgttttgctttggggataaggaaaagatatctggttgagcggcattgaaaacagtgtagggagagaaaaactcattcctggctcacccttttgagtcccactatctcaataatctgatgttatatgacacacacacacacacacggaggaatcctggaagactccatatcaaggtggtgatgaaggtgaccagtgggtgataggattataggtgtgtgtttatttatttattttaattacctttttttagagacagggtctctgtcatccaggctgcagtgcagtggtgtgatcatggctcactgcagtcttgcactccagggctcaatcctcctgcctcagtctcctgagtagctggagctgcagtcatgcaccaacgtgcccaactaatttactttattttattttttattttttgttaagatggaatctcactttattgcctaggctggtcttaaactcctggtttcaagcattcctcctacctcagcctctcaaagtgctggaattactgcacttggccctattatatttttaaaaaatttcaatagttttaggggtaaaagtggctttggttacatagatgaattgtatagtgatgaagtctggatttttagtgtacccatcacccaaatagtgtacattgtacccaatgagtagtttttcattcctcacccccacactgtccccacttctgagtctcctgatgtccattatagcaccctgcttttgcgcacttagagcttacctcccacttagaagtgagaacatgtggtagttggttttcccttcctgagttacttcacttaggtcagtggcctccaatttcatctgagttgctgcacataacatgatttcattctttttttgactgagtagtagtccatctctctctctcacacacacacatacacacacacacacacacacacacacacacacatttatccactcatccattgatgggcacttaggttgcttctatatctttgcaattgtgaattgtgctccaataaacatacatgtgcaagtgctgttttttctcccttttatccttcttttcttccctatgcttccataggtactgagaaagagtcttttttatataattatttcttttcctttgggaagatacccagtagtgggatggcttgatccaatggtagatctgtttttagttctttgagaaatctccatattatctccatattgttttccatagagattgtactaatttacattcccaccaacaatgtatgtgttccattttcactgcatcggcaccaacaacggttgttttttgactttttaataatggccattctggctggggtaaggtggtatctcactgtggttttaacttgtatttccctgataattagtgatgttgagcatttaagaaatatatttgttggccatttgtatatcttcttttaagaaatatctcttgaagttgtttgcccactttttaatgtgattatttgtttttttttcttgctgatttgtttgagttccttgtagcttctgaatattagtcctttgtcagaggtatagtttgcaaatactttctcccattctgtaggttgtctctttactctgttggttatttcttttgctatgcagaagctttttagaataattaggtcccatttacttatttctgttattttgttgcatttgtttttggggtgttagtcacaaattctttgcctagaccaatgtccagaagagtttttcctaggttttcttctagaatttttatggtttcaggtcttagatttatgtctttaatccatcttgaattaatttttgtatatggtgagagataggaacccggtttcattcttttacactacatgtggctatccaattttcccagcactgtttattgaataggatttcctttccccagtgtatgtttttgtttgtttggctgaagatcagttggttgtaggtatttggttttatttctgggttctctatgctattctacttttataccggttccatgctgttttgattacaatagcctcgtagtataatttgaagttgggtaatgtgatgcctccagatttgctctttttttgcttaggattgctttggctatttggacccctctttggtctcatataaattttaggattggtttttctaattctgtgaaaaatgacattggtattttgataagggttgcactgaatctgtggattgctttgggtagtatagtcatttttacaatattgattcttctaatccataagcatggtatgtttctccatttgcttgtgtcatctattatttctttcattagtgttttgtaattctccttgtaggggtctttcacctccttggttaagtatattcctatgtattttatttttattttttgcagctattgtaaatgggattgagttcttgatttgattttgagcttggccatcattggtgtatagcagtgctagtgatttgtgtacattgattttgtaacctaacactactaaattcacttatcaaatctgggagatttttgaggattccttaggattttctaggtatgagatcatatcattggtagaggtagtttgagtttctcttttccagtttggatgccctttatttctttctcttgcctgattgctctgactagggcttctagtactatgttgaatagaaatggtgaaaagtgggcatccttgtctcattctaatttttagggggaaatgctttcaacttttccccattcattttgatgttggctgtgagtttgtcatagatgattcttactattttgagatatattcatttgatgcctagtttgttgagggattttatcataaaaggaggctggattttattgaatgctttttctgcatctattaaaatgattacgtttttcatttttaattctgtttatgtcatgaatcacatttattgacttatgtttatttgttgcttacatctactttctaattttactataataaacatgtataattttgttatcagaaaagtaaatgtaaaagtgagttttaattttaaaacttgggcctaagtcttcctgcctcccaagcccattcccttcctgatatctggggcttccctcctcaagcctgctctgcaggataaggggatacagtccacatgcctgctgctggtttggcccatgataacctccatgggcaatgtctgagcctctgctgttgagttttgctttacacactcctggcaaggaaaggatggccaacatggcttggacatgggttgctgataattggtgatgtctcatgactggttctgcctggagggcttgctgtaagtccctgataggaggaacatggacctgcacaagagcagaacttatctgacactgaagaggacacttcaagaacagattatcaaagtctagctcagggagaaatatactttagagcagaatgaggaatggcgaggcagctgagcttagacacaagcagaaggaaatccatggtgagggcacaggcaaggaaaggggctgagagagcattagtgggggcagtcaggggcagtggtcaggatgctcggatgccagcgtgaacaatcgcatcaagattaaacaccatgaggatcgttagacttcctgtcatatgtctccaggtggtgctccaaatatcctaaaccagatgacagcacccctccaccctctgctgtataagcacatctgctctcctataatcattcccacatagcaatttatcatttttattgatttttcttcatttaatacacgtataagtgtgtcttttatttttaaaaatttgcattcctttaattgctttggagattgtgcatttttctctctgttgatttactctgccaataaacatgtaatcctaccataagcatgttttacttgtgtaatcaaccaaaataaaaaatttaaaaaggaatcactgactatgaattagacatgtggataggcaccagggttgcagacatggcccacgttcttgcattaacttgcactgtggctggggcattggatgggtacattaaaaggattaaagtaatataaggcagtatttattaagtgttgagtgagcactacagaacccaagtgctgagggagtttcatgcaggaagagatcaagagtaacacagagaagaagaatagatcaatttagcgcattcatttaaaaattcaccttttgcataaggggatgtgtcttttgtggggaggaggggagttccgattggcagtttgttctcagggagcttgaagaagagatcttggagaggagacgcagagaaaacaaatgaagaaaatgtcaaaatggaaggggttggcccggctatgcataccttagttagcttaggtagagtctaaacttttacaagtggtttcaataggtgtgtttggtctgggttctttgggaggtatcataggagaatgaaggcagggaggacgcttccagcaccaaaattcaaagggaaatgtattttacatgcatagcattgttttactctctttccatttggagcatatcttaaaaattccatttggagcatatcttaaaaaacccatttctctgacaatggttctaaaagggggaaacatcctttgcaacagaatcattcattctctcattcatcaaccactgattgtgtactaagtgtcagacctgatctccatcctgcctggtatggcactagcttctgtcttgagacaagcattgtgataaaccatgaccaaaaaaagggcagttttataaacacaagtctgccaggctttcagcaattctaaatttccttttgcaagtcaggctggagttaatggctctttcctgcagcggcggagatgacagggctctcccacagtgctgagcaggcagtttgaaagccccacttcctgtctctgcatgggcgagtgtccactggaagccactgagaggaaggagggaaacctcagaaaccggcccctgcctggctgcttcaccctagaaagcccaggcagaggagggaaaggtgaagtgctgaaaaagaataaaaaagggggaacatgaaaaagagcaagagcaggaaggaggcagggacgggaaaggaggggaagcacggaaacagccaatgtcaaggagaagaaaagatggctggtggaaaggagcttccaggaattgggacacagccctgtcttattgcaaaagatggaaaccctgaaggagaacaggaaggaaaaagaaaacaagtccgtctgagctggcagggtccactttctcattctacagatgaggaaacagaggcacagagaggaagtggcttgcccaagggggcagattcttgaaaggatcatctgcactctctctcccttaatgcattcttacctcttctttactcgtgagtcagtcctgaaggacaagctgcctgaagtcccacacagatgggcctggggcaagcatcaaacatcctgggggccctgggtgaggtttgcttttaaattccaggtcagggaaaggaaggtctttaagttgtctgctctaagcttagtaatccccctcagagttatgggtgcggtgtctggggtagccgttgcgtctctgggcaaataccctggagaatgcagtgttggttgtctgagctggggacagagtgacagcatagttgcatgcagagctggaggctcctgcagctgtacaggtaaggtgctgaaattctccaccaacccttcctctttgcccccagcaccacgaagataaccctctttgaatatgtggaagtctgttctccaaactttctaacattctcatgtcagtcttaatagattcagctcagttactgcctcctccaggaagtcctccttgtctgcaaatcggctgcccaccatgccggctcactcatagttttaactctgtatctttctaatatgccttagcccactctgtcaggattccagtcagcttccttctcctagactaggagttgcctcaggccaggaggaccagccttgttcatatctgtaccctgcaaacctgtcaatgcccaaacctgctcagtgctttggagtatggaaccagccgtcaatgcaggaatgttacactctaagagttcccaaaggtagagagatgagggattggtgctggaagtgggaggttattctaaggatgggtatggcaggaaacacaattatagttcagggagtggagtgtccaggagtgggaggagaggaactgggagaaagagcagagagtgaaagtgagagcgggcacaaagaaagggaaaaagagtcagggatcaaccaaagtgcatgcttccttttcagccctgccaggatgtgcagggcggctgctgtggacgcgtcaaggctcagcctcaaacatgtcttcttccttgacttttgtctatcattctaaagctaggtcatttaaaaagttcttttgttttctttccaccgatactctgatttctgacattcgccaaaaagaggtcaagaccctggcataccgccctactaagattaaaataaatattatccattgaaactgttattttttccttaactgttatttgtagagttaaagattcccatgatcgcgctggctctaacatcatttttggctcttttgagatcaaatttgcaatttgatgcaaaaatagctgtgacgcatatgtgtctgtatgtgtgtggttaggagattttttatcattacatcttcttttgccctgcctttctgcctttctgtccttttaatttgcgggcttttggcaaccacagcacgggtctggtttcctaggagtttcttttgtaggatcaaaccgctagttggctcttggccctgtgatagggccctgggctaacttattgggaaaatgttgctgtaacccctgcccagaggtgcctgtgacatgggccgccatcttctcctcttcccttggcttcagccccacctagaaacctgaacaaacattttccttgacatttcataaagtgtcagtggctcctcatttagcaaaatacatcccagggaagttcaaaagtgaaaaaaggccgtaacttcttcttcttctcagggacctacagaaaatatgtggcacctcggcagcctggcctgcagcactcccctccccatcggtgagtcctgctacagtgggtccaggtgtctggacgcccggcacgcacggctctctgcagacctctggacagtaccatgggagccgcacagtccctgcctgttctgtccggcagttcttgtttcccagcaccctgtctcaggtgagaggttccctcttctgctgggcttctcctccctgctgtgaaccccaaatatctgaggcaggtcaatttaggaaccttattttgccaaagttgaggatgtacccatgacacggcctcaggaggtcctgaagacaagtgcccgaggtgatcgcggcacagcttggttttatacatttatacagacatcagtcaatatatgtaagataaacattggttcggtcccgaaaggccggacaactccaagtggagagggggcttccagttcacaggtagataagagacaaaatgttgcattcttttgagtttctgattagcttttccaaaggaggcaatcagatatgcatttatctcagtgagcagaggggtgacttggaatggaatggaaggcagttctcagtttaaattttccctttagcttagtgattttggggtcccaagatttattttccattcactctgcagacaggggcttctgtgcatccagggagcccctcctcacagaaggaagcaggccattaatgagacccaatccagcttcaaccacctggtaacaattaggacatcacttctctgagcaagagctcctgcctgtccatgagttatcaagacattccaattgttcctccacatctttgacatgaagacttgagggggtcagattttccagggggcttgatggcatgttctcttcactgttccctgccctggtcatccaagtgacccttggcagggaagaggccccgagttgcagaatctctgttctcacaagccattgccaacccggagagtggctttgccactattcctagcatgttgttggctatttcaggaatgggagtatttgacttttccctttgcagtgattgctgcaaggagaggaattgagagactcaagtccctgagataaatatttatcaactattactgaaagggagtatgtcaaagaaaaaatgtggagaaacttcagcttgaacacatagtttaaatccagcttgggtgtactccagtgggcatggatgtattactgttttgcagtgcattcttctatgatcaatacacagaagcaaacaggccacgtgggtaaacagtaattttcatttaccagggtgaatatggaagtcctcttgtttccatgtcatgatgaaggaaagcaaggaccatcttttgccaaggaacagtggctgtgggggaactgaggagatggaaggacaaggcagtcaaaagctttggaacaactctttttttgagatggagttttgctcttgttgtccaggctggagtgcaatggcacgacctcggctcaccacaaccgctgcctcccaggttcaagtgattctcctgcctcagcctcccgagtagctgggattgcaggtatgctccaccatgcctggctaattttgtatttttaatagagacgggatttctccacgttggtcagctggtcttgaactcccgacctcaggtgatccacctgcctcggcctcccaaagtgctgggattacaggcatgagccaccatacccggcccttttttggaataattttataggttttcaaactattacacttacctttttatataagagacaggacatagtcactgaacaatcactccagattttaagtaagtccaggatgggatgacaatggaacaaccatgaaatgaaaggaagaatgtgtcactggtatgtccacacgtctccaaatctctcacctctgtcagctgcaaacagagcctgaaataaatgtttcctctgtgcacagcctccacaacttcctccctccacgtttctcactcactcctctccagcacttctctccgggttctgcttacaaacttgaaaccggctatgcaaaaattataactgtggaaattatgacagtgaaagagatcagacctaaccgactccatcttgcttctaacctttaagctgtccttgttcatttttgggctgaactaactttgggaaggaattcagttcatggtagaactctgaaacaaaattgataatagccctttcctgaaaagacccccttcttgcctggggacaagtctgccattgtaggactaacaaattaactacaagattagaaattaaggtttagggttcatgcagcctccagttccaagagtctaaacctccccaaattgctcctggggataacatcactgttgtaaaagctaagaccagtgcttgagatattttgtagaccctgctctggatggatcagctgacaccatccagactggtaatttggctcaaccagctctgccatcccacccaggaacagaaaaatactcacttcatcaccccatgagtccatctctaacctgaccaatcagcactccctacttcccaggcccctactcgccaaatctgcctttggaggcagataacaacttatctttaaaaactctgatccctgaatgctcaggagactgatttgagtaataataaaactccggctctgcatgaattactccttttccattgcaattctcttgtcttgataaattggttctgtctaggcagccagcaaggcgaaccctttgggcggttacaaactcatcctctgtggaagagtaggagttcatggagaaattggttgcaaattacaaaattttattgtaaggtcaacttgtcccagtgtccgtctgtgcagcgaagggcccctgcatggtttagtgattgcaagttgagcctctagggtcaggttgtctaggtttccatcccagctcattcacttattatctgtgtgttcttgagcaagctccttaatcaattgaggctttgtccttctgtttgtataatgatgagaataataacctccacaataacctcatcataaggttgttgtgaagatggatcagataatatatatgtagagtgcttataacagtgcctggcacataaaaaatgctcaaaaatcttaagtgttattaataataaactgacatatatttcttgagcagggtggtggtaaatgggtgttctttttattaagctttaaagtgtgcatagatcatattaattctttttatgcatatgatatattgcacatgcatgaaaatacatgcattaaaaataaatgagcatttatgagatttagtttagcagtcacatgtcccaggattacaagccagcaataatgggttggaaaacattccaacccattccaaccattggaaaacattccaacccatcactggacccatgtgccaaacaatggaaccgcccacaggttctcattcttggttaaaaaaatatgattattacgggaataatactgattccctaagaattaatatctgagcaagtttcttttttttcctgtcttcttggaagatcagcaggttctagattcaatggagtcactaggattgagccaccagtatacgccagtcctctccagaacggccacctggtggtgggcactaaggcagtctcagatgaggactgattgacttttgtgtgaactcaaactgccaaagtccctccctcaccttgcaaacttcaaagcacaactttcaaagcactactttctttcttggctctcaattctctgcctagaaaaagggaggtgttggcaaggatgtttgtttagttctgggcatcagtcaatggtacccagatcttgctgaacagaaaagacacagatttgtttctctgaggcagttggtagtgcttattgcttattgctctcaggggcttctgcagcagtagaagggccctcttcccctgccatgccacactgagaggagcatccttggagtcatggttggaatctgtttttgttatgctagtcctcttccgcatgctagctgttgcattgcagggatatgtgtacctgtttatcttctccactaggctctaagaagccaggtttcttaaaggaaggaagctgatcttgtttatcttgaagtcctcacagtgacattgctcagtcaatgttgagtgtatgaatgaataaacgggaaccatcacgaaaaagccgaaaatacagtggaaagactggatcataaaatcttctaagcaaattttttttcctcttacactccatttccaaatagataaagtattttttaaaatcctatcagaatattctaacacactgagttgacagaatagagatttttaaatgcagtgtcatttggccagccatttgtgagaatttataaatgtttcagtaggttgaaaacactataaaagcaaggactatgttcatacccaacagctggcacttagtatgaatgctaaatgaaacattctcttctctttcaagagtcagtccaaccagtgaccctgacaagaaggaaggcacatttaactcaatttaatgaactcttatagagcatctccttctccaagtgctttgctaaggatggggtaaaaacatgaataagtcttggattctgtccttcaggaattttcagtctttggaggcagatacatttgcacccaactattatcctaggcagagtgtgataagtacgataatagcagtaaaagctctaagttaggcaggagaggaggagctcgttaaagcttatggggcctgggaggctttcggcggagtaaactccagggggacagctaggcatctggctgctggaattgggaggaggatcattttaagtggctacaactctgggtgcacaggactagagggtgagggccaagatgggaaattgtggcagccatcttccacactgggcgcccgccgacccttgcttcctggtattcatattattgtgtagtgtcccccaacattgtatcagggttggcctgtgtgaccaattgcatatggtgggaatgatggtgtgtgacttctaagaccagttcatagaagatgtggccaattcccttactgtctttttttttggcaggggagtgccgagtttcacccttgtcgcccaggctggagtgcaatggtgcgatctctgctcactgcaacctctgcctcccaggttcaagtgattctcctgcctcagcctcccaactagctgtgattacaggtatgcgccaccatgcctggctaattttgtatttttagtagagacggggtgagatcaatgaggcagtcaattggccagcctggttttgaactcctgacctcaggtgatccacccgcctcggcctcccaaagtgctgggattacaggcatgcgccaaccgcgcctggcccttactgtcctttggatcagctgctctggggctaggtcaatccttcatgtgactgcagccccagccaacatctggactgaaacccatgagacaccctgagccaaaaaagcccagctaagacttcctgcatttctgacccacagaaactgagaaaagaaatgttttgttgttgctttaagccactgacttctggggtcatttgttttgcagaaatagatagcagatacagaaaagcaggctggtggaacagtgtgggaaacaccttgattttcagggagttgcactttgtttatgtgcaatggtgcactgtttttagaaagacacaaagatgataatactggtgatgggcataatacgggttgtcaagaggagtgactgaggcggggataatttaagaggccacagcagtagtgtggcaagaggtaatgagggaattgaacttggtgggaatgggtgagatcaacgaggcagtcaatatgggcagtgagtgtgaaggagctgcgaaggatgattctttggttttgagcttaggaacatgagagaaccaagatctcatttatccaaagaggaaacacagaagtgagcccctgtttgggggcagggctgggtaggaggaaaagagtggagacgtctatctccccaggaagagagccccctgcttccagatcccagtggatggcagggcactcggctcattcacagactgggctcgttgagaaacctttccctggagggcagggctgctctgtttcacagcccatatccctcatggccaagtgttcctcgagtgacagtctctgccatcaatatttttagcatgtggtctttcagagactaaagagtggcatccatctcctgaaactccttccccagctgacagctggtgacccgtggaggagggagcttcagggagcctgatgggcgagagtctgttccaatgccaatccattggaagagatgaagtcagacccgagtttgatagaaagcctacttcctcccttgtatccagctgtggagacctaccaacatcaatgcaaaccagaagctaacacccagttcatatatcccaagtggaaggaagcttctcgtggaattgtcttacatgacagtaacataaatcctgaaggtaatacttggccaggtaatgttagaaaagaacccgaacataggcattgctattatagatcctaggataggcctgagcaaaaactgtctgggattcataacatgcttcgttgcaatctgatagagggagtgagatccactccaaatggagtctgatttggggcaaagcaaagagtatggaaggaaacttgagaaagggggacagcttctcaaatggagtctggccacagctggggctggaaaagagacatgactgcgcttgcagagtggtgagaatttgctgctagaatttttaagttgtgtgttttcatttttatgataatgtaaactgagataagcatattctctgctatcccaatgagcccctcctctaggaggactaccttgccaccttatccataaatgtgtttataaattattttgatgccagctggtattttttaaaaagtggttttggactcacaaaaaaaaccatgatggatttaatacataacaaagcatttgtgtcaagtgaaggccaagtaacatcttagcgtcctgtgtgagcgaaggtgtcgtggcagttcaaacaagaatgccgatgaagctgcccaggatggccaaggccaccttggtgtgtttgaggggaattagagtttagaaaaaaaaaaaaaggcacctgacactctgaactaatgtggttacctggaattttggggttttgaagctttgcatttaatttgcagcttatggcctgaaggaaaagacaggtgaaatgcatatcctgggatgagtcacctggaggagagggctgggaaggggctgagctgcacatgctcagatcttctcccaggcttatcgacccagtgagtcaagtcttcttccaacgggatagagtgtgagagagagcagggaacagaagccagagtctctgttaaatttctcggtacatttctgttagagaatggaagtttctctatcgtaggagaccttgagagcctgggatagaaattacccctttgtcatgtattttcctcccagaaatagcatggccactgtcactgctaagctggagtatcatgagcacaatttctctcactttctatacccatgcctttctaggagattggtggctccatcaaaaaggagttaaaaagaagcagcactattttgtggaatacaatcatcaccattatcaccatcagcaccaccaaccagcaccaccattatcaaaagcattcacctggtgtctgccttacaaactgcaaactgcagtaggtatttgtaatagaatgtttcctttcccccttgggatctgcagaaaagctggagaatgttttggtatcaacacactaggttgcattgctaatcatgtgatggccccatgacagtctctgttggctggtgtagttcaggtggacgactgcaggattttgttcttggagcctcagttctgactgggcttggggtgtaaaaggtttgggagccagatgacaagagtatttgatgggtagaataatgggttcatccaaaagatcaccagaatggttattaaatagtacaaaggaggaatttactggtaataccagtttgcaaacagagaagagagtctccaatgtggactgaaagtgctctctctttgaagaggggaaggacagattgggttttatgcctcacaggactggtaccatacatattcagcaggtttttggggaaaatctatacatatttataaggtgagctgatgcctgcataatagataaacatatatgtaacatacttttcatattcattttgggactgggttttggcactaaaatttgtggaatttggctctttatgttaaaaggtgaactagaggacacaaagacggtttgtgtgcaccctctataaactggctgaaactggcttaaggtctgcaactgcttatccaaaaagaatgtttgtaaggccaggcctctgtccagtcagagttgtagtggtccaggttgtaaatcaaagtttatagctctttttgttagagagttcagctgtaggaatttagaaatttgccatgcctgccaggccctgaacctttgacccataggtaactttatttccttaaccttagggtcagtcttagttgatatggggcatctattctggtatctcagatcctatggtcaagagaaaagatcctccacaagagggtcctatgtggctgcaaaaactgctctgagctaaatccactcaaaatcactgcaggatgtcactactagaaaatagggcagggatagggatccccttcccatgctgccagaaaatgcctgatagcttacctcccccggcccttgaggctcccttggaataggcacatgcaatcccatctccacccaatagagcttgtcctagagctcagttttttcccatagttttcccacccacttgcaccagaaaatctaataaagtcatgtgattaatacaattcattttatcacgcttctgaagatttaagagagagcggtcacattggattccacagtaccgaccttctgacgattcttcatttcacctttatctatttttatttttattttatttttttttcgagacggggtctcactctgtcacccaggctggagtgcagtggggcaattacggctcactgcaacctctgccttctgtgctcaagcaatcctcccacctcagcctcccaagtagctgggatcataggtgcacatcaccaagcctggctaattttttgtatttttggtagagatggggtttcaccatgttgcccaggctggtcttgaacttctgagctcaagtgatctgcccaccatagcctcccaaagtgctgggattactcacgtgagccacctcgcctggtccctttcacctttattatctttgcctttaactctagtgcttcctccctgaatcagttaaggattgcatttggctgcattaacagaaacctgactgcagaagcttaaccaaatagggtagtttttaaagagagattgcttacatcacgcaaattgcacaaattttaagtgcatagttcaatgagttttgacaaatgtagaataacatagctatataaaaccattccatcaaaaaaattttatcaccataggaaattgtgtcctgtccctttcttgtcaatcccaactcctccccacaaggcaaccttcattctcatttctctcaccatagcttagttttacatgtttctataatacagcatcatataaatggaataatacagaatgcaatcttttgtatgaagcttcctttggctcaatgtaatgtttatgagattcatccatgttattgaatgtatcagtagtgttttcatttatatttcctagtgttctattgaataaatatactacaatttgtttatccacttatttgttgatgaacatttggaccgttggcaatttttgcctattatgcataaagctgttaaaaaacattcttgtacaagtctttcatttcatatgtttttctttttctgaggtaaataactacaagtagaattgttgggtaataaataggcatccatctaatattataagcaactgcacaacagtttttcaacgtggctgtactatttcactctcccaatagcaacgtatgtgttttccagctactccacatgctcactggcatttcctgttgccagtttaaacatttcagccattccagtggatatgaaatctctctggctataataattgtatttctctgatgactaattatgtcaagccccttttcaaatgcttatcagccacttctatactgtcctctgtgacatgtccgttcaatctttttgctcattctttaaaaacattgggttgtttgtctttttcttagtttgtcttttgcttttcatttataggagtacatatcttcggaatacaagtcctttgtcagataaatgtattgtgaataattttctcctagtttgtggtttgccttttcacattcttaatatcttttgatgagtggaaactaactttcaaattatgttcagtagattaacttgtttttgttttgttttgttttgttttttgtttttaacactgggtctcacttgttgcccaggctggagtgtagtggtgccatcatggctcactgcaacctctgcctcctggactcaagggatcctcctgcctcagcctcccaagtagctgggaccacaagcacgcaccactacacttggctacttttttatatttttggtagacacaggatttcgccatgttgctcaggctggtctggagctcctgagctcaagcgattcacccacctcagcctaccaaagtgctgggattacaggcgtgagccaccacgcccagtcgagtagatcaagttttaattttatggccagtagagatctatttcaaggctctctattttgttctgttgctctatttatctacctttatgccaattttcttctcttttgattcagatagggttataataataattattttttccagggattagatggaccagggctggtgaagttgttcaagggagtgatcaagagcctggctcctttcatccttctgttccatctcctttggctcatggattttgttttccaagtggcaagatggcgcctccacctttggtatcctattttagttcctggcagaaagaaaggaacaggctaatggccctgatgagtctacccccttttaacaggagaaaatttaaaaaacaaaaaccatgaaaccctttcccagaggcaacaaccagaattccatttatctttcattgaccagaacagaccacatggtcactggtggtggcaatggagactggggagatgaatatttttaaggtggcatattccagaagaacactgtgcactgattgcattaatgaacccattaatgtgccaaggggaggtttacctatgagcatgggcaaattagaacccactcttggagctgcaggtgagccaatcccacctaaacagtgtggatgctacaagatggggaagtaaattgattctattccataccctaacctctctccaagatgtattcttaaaatagaagagggaagacagaagaaaacatccagaatatatttttattgtcttttacttcttcagtgcattttagatcagtgcttctcaatctggcaaggggcatgcaggaggatgtgagttttatcaggaaaactacacaaccccccaaccacaatgctacccccactcctgtggaccttctttaagagagactcactattatagatggagttgatacgattttaagagaggccatatattatttgctttctgtcttgaaaaacttgtgatttttctgtattgtgctactgccaaagagaatagaaacctgactgaggtgtcaatgtttatgtaactgatttcatgtactttctgtagttctaccatttctgatggttaaaaatttcttgtgtgtgtgcagttggggagtgtgtcctcctccttctgctcttataccacacattagcacatcaaaatgctctaatctttgtatgattatgtggcatgtggtgatgcagcctcacagtggaaaaacttctcttgggccattgcaaatgtaacatttctttcaatcagatagtgccattaaggatttcattatggccgtcacatcctgtgacatctctaaacatgcagcattagggcctaagtgcagccctgcaggtagagttgccaggtttaacaaataaaaattacacgctggccaggcggggtggctcatgcctgtaatcccagcactttgggaggctgaggcaggtggatcatttgaggtcaggagttcgaaaccagcctggccaacatggtgaaaccccatctctactaaaaatacaaaaattagctgggcatggtggcaaatgcctgtaatcctagctacttgcgaggctgaggcaggagaatcacttgagccctggaggcgggggttgcagtgagcagagatcacaccattgcactccagcctgggtggcagagcgagattctgtctaaaaaacaacaccgtatttggggcatgctgatactaaaaaattattcattgtttgtctgaaattaaaatttaaattgggggccctgtattttactgggcaacccatttgcaatatcagcaacaatctcttattcagaccactgattaagtgtgcaaaatttgaatctctgaacagtacctatgtccttgatatcttaaattaatgagtgtcttagacactcaaagcaggaggaagcattatggcagatgtttgagccccagagatgtccatgagcacagcatagagctcagagccttctttattatttgcttcacgacagagcaaaggactgcagcaggttgactgatataaaagttttaccatgtctcacagcaggcctttgctcaagtttccagtaaggatattgtatcatttcttgcctgcagtacttgtaaatccacttacactgcctgctgttgagtcatttgtttcgtcttgagtagcatgtcatccttgttcctagaagatagtgagtttagagacagtagccaagcaacagcagagcagcctcaaccaaaacgattttccattttggtgggatgaattgaaacacaagcatcttctatccaggggagatttggggatcataaagaatcaatctgagctggtaccaccatattggctgctgcattttctagagttgccgtaactagtctcacaagctgggaggctttacacaacagacatgtattgtctcatagttctggatgctagaaatctggaatcaaggctccaggggagaagctgctccatggttttctcttagcttctggtgttgccagcaatccctggtgttccttggcccgcaggcggatcactcccatctctgcctccattgtcacacggcattttcccagtgtgcctgactctgtgtttcttctcataagaacatcggtcatattggattacaggcccgtgctactccattatgacctcatcttaacttaaacaattacatctgcagtgatcctgtttgcaaataaggtcacattctgaggttccaggaattagaacatagacatatcttttgggaacaaaattccagtgataacagtttcggagacagactagtcctggagtttgtaaggtgagccaggaccaaggtgccaggattctcattttgtaaggtccaggaacaaagtgatgttaatagaaagaacatgtttttgtttgtttatttgtttttgagacagtctcactccatcacccaggctggaatgcagtggtacaatctcggctcactgccgctgccatctcccaggttcaagcgattctcctgcctcagcctcctaagtagctggaattacaggtgtgtcccaccatgcccagctaatttttgtatatttgtgtgtgtgtgtgtgtgtatatatatacacacacacatacatacatatatatacatacatatatatatacacacacacacatatatatatatataaaatatatatttcttttagtagagactgggtttcaccatgttgcccaggctggtctcgaactcctgcgctcaagtgatccacctgtcttggactccctaagtggtgggactacaggcacaaaccaccacgcccagacagaaggaatatgtttccttccagtctcacttgactggctgcttccctagataacaacagaggatgtctgttgcagttctcattgctggggagtctaaactggaataaaacacccactatctccatcaggcttgcactagagcccagctctagctggagagaaagaagctaacccgcacagacacaggactgtaggcagggagcatccgggggtatttgggtcctggctctgatgtgcctaaggccaacttctctctggccatgctggcgtgcatgagctcactaatcttcctttttgccttccattttctccaatcctgacttagcaaaggttgggcaaaagagactctgtgtgagttcgagcaaagcctgagatgctggattttccaagatacgagaaggggctgggggctgggtgaactggtggtggaggagggaaggattaatttcccaaggaggggaaggggccaggacatcaggccccggggactttgaagagagggtcgtgggtaggaggtagatcaagtggagtgacacaaaggtcaggaaagaggaagtgtccacactgtccttcgacagacttgagtctatgggacttcctccctgcacggtacaaggaaatgagtaagtgagataatgttgtaacttctggccctctgacattgcactgccccgatgtcacagttggaaactgtacctgcccccatccttgtctggggtgtgtttggtctggggagggctggtgaagcaagaggtactcagaaaaaggacagaaattgcttcctattatctgggcatttggaggtgaaggggtcacagctctggcaaagatggggttgaaagggcccggactccagggaggggcagctctgcatggcctgattcctgcaccccacctttgccccctcacacctcctctcatctcccgtttttgaagaggaggaccctgtcacatctggacaattctgcaagaactctgtagaactgacttcactgtgaaccaggctccagaagtcaacagaaacaaaaatgctcacatttaatcacgatgctccctggcatacacagaagactctgaaaacttctgaatttgggaaatcctttggcaccttggggcacattgggaacataagccatcagtgctggtgtgtgtgtgtgtgcgcgcacacgcgcatgtgtgtgcatcttctaccatgcctcctacaaatttgacctgggcccagggccatgttcggtggtttttaagaaccgaggctcccagaagcagtattgggcagctagagtggccccaggatctatatcaaactctacctgtttctgaaccaaatttcttctagaattttattccataaatctgaattatggtgtcagactcctagcatacactaaaggaactctctgccttgcattaaataacaggagttacccctggaggtaactcctagccctggctctttagagaacagatgccgaataggcattaggggatgtgatggatgtgctaactttcaaaaaaaaaaaaaaaaaaaggcctgagctgagtgctcagagattcacaaaaagctgacagcatctctctgttccattggaagctgggtgatcctttctactctttcctgagaaaggcagttgggcaggaaaaagctgtatctctgtcctcactgagagggtttcccagtctgagggtgaaggatcaggagagggagacctgacgggtcgatgtggggcatcatccacttgagtgagaaccagagggatcccgtcattgcccagggcagatgctccattttggggggcatcattcattctttcctgttctccctgcattcctctggctcctgcccaggagaggtggccgctggcaagagagcttggtggaggtgggaggtgggaggtggggggtggggggtggggagttcttgagccaggacctagcgcatagtctccagcctgctgatggctgtcttggatgcttcaaaggggagaagatcctagatgtgggaaacattggtgggcgttctgctggggcatctgtagcctctgagaaggctaccagtctctcctaagcttacgccgtcacaccctgggcacttgttgaatgactttacttagcttacagcctctggttcctgttgggaaacttagggcttgccacagtgttcattttcctttgcgggcaactccgttcctggcacttatcatattacccactgtactccccgcttagagctgtgtcaaggttctgagaatctatcccttggcttggaaggggtcatctctctggccagatcatttcctgataggtcctgaggcaccacaacacataggaggcttgtcctctctctggggttcactgccttgctccttctccaggtcaatatgtgaccttggaccggttgcttgagtcccctggtcattcagaaacaattgggtttccctggctttggagcctggcagcctggctttgagaaccgggctttaacttgtcacatgactatggccaagttcctggggctctccaagcttcacttcctctgtaaaaagggcaataatataatacctgtcttattgggttttgtccatgttagatgagacattgggtacaaagcacttggtcccgtgcctggcacatttactgcacttaatgtatgatagttttcttattattctaataaacaatatggctttgggagtatagttctgccacattgcagtggccagagtgaaggtggtgagtgccttctggggccctgggagtcaaggttatccgcatgccctttcttgcttgctcctcagtgtggctgcctctatgtccacaccatgcagatgcaacaggtagtttgaacctctgaggcccacagtgggatggggaggcagggacatcacttatggggtgggaagtcacccattccccaggaaatggccccagctgccttttccatgactcctcttgaaaccctgtggaggccacattcgtgttggggcggtctttcccatgaggatatgttcagatgccgaggcattttgaaaagccctccatagagtttcctttcataacacatgatcatccccttgggcttctggttttttttctttcaggaccttattttcaggcaagtggcctttgacctctaaggctgtcctttcctagctaccgaatccagcattcaaagtgatggaaatatgtatatatagtaatagtaaaatatcagcacttaatggcctgataagaatgtcactgcaatgctgagtttggaccaacatttgcctgctcctgccattgagcccgggctcccctccagagctgagctgctgcaagggatctgagtaactagggctgtgtcagagtggcgatgacagccaccacatgctaaggaagagatccccaaggacaaggagaatcccacgtggagctacttgcttctttgtcagtcttgtttttcttatttcacaaccttctaaaacacaatctctcaacctctattgttagcttgcatttttcaatcatgagcacagctttacctggctccatgctttgattgactctacctgccaacactgcaacaacagggaaagggacaccggcctcataccattagatggtgtgtagcctgggcatgaggataattaaaaactcccaaggggattttaacatgtaacacagtttggaaaccattgatgtaagatcttcttactcaacatgtgctccaaggagctgttgtatcagcttatcagaaatgtagatcaggccgcacttggacctgtagaatcagaatctgcattttatcagattccgacattatttgtatgaacattagcttttgagaagtgttgctttaagagactaagggggtcaatctacctcactttgcagctctgtgttccttagtcattggctaaaatatcagcccccctgcaatgagccatcctcccttgtatagtcagtgatggcctgtgaacctttagccaactggaagtgggaggggacacagtccacaaaacactatcctgacttttgacaccaactacaagtcaaggggttccccaaaccaccctgagttgtgataattcgctgggagatctgacagaactcactgaaggttgttatactcatggttgtgatctcttatagggagggaatacagattaaaatcagccaaaggaagaagcacacagcacagagtccaggacagtgcctgacatggagcccctacggtcctctcccgtggagtcacggacagcgccactctcctggcattgatgtgtgacaacacacagggagtgttccccaccagggaagccttggtgtccagggtctttactgtggctctgtcacatgagcacagctgactgcccatgcggccgatctgttcccagactctccaccgctacacatcactcacagtccctgctctaaatcacacaccatgacccaatgtccccgggcaaatgaaaacacctctagcaggcaggacgttccaaagccttagagatcacctctcagaagctgagggcagaagccagacctctttttgggcagggttaaattctttattactgtttttgaaaaaactcccaaattgagtttttcctcttcacttacagcagcataacaacaatcatcaatgcagaagacttctgcgagcaaaggtgtgggggaaaaccccaagcagtggacactagctggtgtcctccaatttgattctgatgctgtctactgggagatagtgtcagatcctcaagcctaaaccctccttctcccagtcagagggctggcctttggaacttctgaccaatccacttcaagttgaggttccaaccactccgctctttgggtttggttgatttgctagagtggctcacagaactcagggaaacacagctaccagtttattgcgaaggacattttaaaggataaaagtaggcagataaagagatgcatagggcgaggtgtggaaaggtccctagtgcaggagcttctgtccatgtggagcgggggtgcaccaccctctcagtacatgaatgagttctccttcacctgcctatcagcctctacatgttcagctccccaacccagtcctcttgggtttttatggaagcttcaagacacccacattctttccccagagtatagggcaagaccttctctggggagggttttaagacccacagtcagaaaggtggggtggggtcaagattagagtcctgccttgacgggcaggtgaaaggggtagggggagtaggtgagaaaaattctgtttattttttctttttttttttgagacggagtttcactcttgttgcccagggtggagtgcaatggcacaatctcagctcactgcaacctccgcctcccaggtttaagcgattctcctgcctcagcctcccgagtagctgggattacaggcgtgtgccaccatgcctggctaattttgtatttttaatagagacagggtttctccatgttggtcaggctggtctcaaactcctgacctcaggtgatccacttgcctcagcctcccaaagtgctgggatcacaggtgtgagccactgcatctggccaaaagattctgtttttgaggcctgcctctgaggtctaacacactcaacattataacaagactgtagtaagggctatgggagttatgagccaggaactgtggatgaaaacctatcacagatatgcatatatatatatatatatatatatgcatatctataataactccacaactacacactgccttattgctcagttcttctctccatgtctctgacccacccttgcccccttcctccatccttttctccattgcatacccatccactgtgccctttggaatgctcacaccatgaactgcaaactctcgtgtggcttcagcctcttctctgaaagttcctctcacctattactttctctggaacctgccatccctgccaccttctcaaaaaaggccttttattctcttcattccacaaagctcagtgtcaaaacatggggtttacactggaagctgaggtcacatcagtagccgggatcagggtcgccctagctgcccaatgcagctcccaggcctcctgtaaaaccttgacctttgaggtcatgacagccctctcctgctatgctcatagctgaccactgaactcctggacactccctcccccaagttcacagagaatgtgggcacatgccttacagtcttcccttgatccaaactactgccttcatcttgagtgacagcagcatcttttggatgtcttggcctgtctagctttatttttttgtgttctgccatcaagttgctacttctgttgccatcgtgcctgtcagcgcagtgcaggctgtggtgaaatcccacgaactcaggcatcacactgaccgggtctgagtcctgtctcagttgtcagctagttgtgcaatgaagggaaagggacctacactttccaagcctcaattcactcatctatggcatggtgacaataatggaggttgatttaaagtcctttgtaagaattaagagttataatagacataaagtgctgtatctggtatacctagaaaacattccataaaagttagtaattgttggtcatgtaatgatgactctctaggctaggatttcagcttcattgcatgcacatggtgcactcacagggcgtgacctctctctgtctcagtaacctcatctgaggaccgggataatcataccgcttcaaagggatgtcataaagattaaataatatgtgtaaggctgcttgcatttagctgcattcaacaaatatttctgtatctttctcctcatttctccttactttcttgcttattatctgctctaggtatagatttcagagaactaagcttgttacaatccttcataaaataaccaggttggttagggcatttccaagagtcaatactgtttagtgactattctctgtttaatctattttgattgtccagggtcatcttttgctatgtcataggttgttggcttcttctagagaagtgagacgatggacaagttccaagtgagtgaggcgactggtcaggatattccgctgaaaaactcatgtcagttctaattcgtgattgtaattcaatcacagcctgagaacagtaggactgtagttcaaatgctctgttcccttttttttttcccagaggataatttttttttttctttgagatggagtcttgctctgtcactaggctggagtgcagtggcgtgatctcggctcactgcaacctccgcctcctgggttcaagcaattctcctgcctcagcctcccaagtagctgggactacaggcacatgccaccacgcccagataattttcgtatttttagtagagacggggtttccccttgttggccagggtggtcttgatctcttgacctcatgatccgcccacctcggcctcccaaagtgctgggattacaggcgtgagccaccgcgcccggcctctagaggataatttttaaatgtgcttttgcatttggaaaatgtgattggcatttttttctaattttctaatatgatacgctgtcggatgctatggattacttaaaccctctggctacctagaaagatctttaagtggttctcaacaagcttcatacgcaatgtaaattgtattatctctcaggatgtgtgagaacatctgtttttcttctaatgcagtaaacatataagggtctcttgggatatcttttaaatagacttaatacaacattcaggaatgataacaaaatataatcacagttgtaagggaatgtgagcatttcatattaataacattggaaccttatgtttaatacagtgttaaaagttgacaaacatgtaggagtcagaaaattcaattaaaattatcacagtaatatgaatttagccacatcctgtgttagttatgaaatccatttaacaccacaaacagtaatatttttagccagtttattcaaaaggaaaacaggaactaaaccactttcatgcaatatatactctgttaatgtggtcaggctaattttgctgggggaaggaacttaacttttgaatatttgaatgcccagtcatttaatctgaatatcctatttccttgcatgttgcaaaatttttgtcaataaaaggcagaaaaagaaatctcttctccatgctcatccctaagagaatgggttgtctgtaccctgagagcattttatggaggggacaaccacttttctaattttccttcccacttctctgtgggcacaaatgctctttggttgaaagagttgtaattcagtcccaagatgaggtgtggttactgcatccctaacctatatctggggaccccacagccacacacatgggggaaatggagcttgtcattcagttctccagccattgcacagggttcatggactcttcgttgatcccaccccacgcttcttctctctgctagccgaacacacttctctcttctttatcaggaggccataggagaagggcattcatttttaatacacatacatctgcatcaagtctaattttgccatgtctcaatccaactgtcaaatgggttgtttgggggctatggtgcttatcaaacatttactcaagaatagccaaaattagccaagcaaggagaacttcagcaacgttcccaaatggccccaaccaagtactgtaagactgaggatagctaaagggtcttgagagggacttctcaggcagtggccccgacatttatctgtttttttaagtgagaaatctgagtaccattcttgactcctcttccttacccccaacccctcactaagccttgtgctactatttagtaaacagaccctcaatgcacaaacttctgtctaaggccatggccaccaccctagtctaatccaccatctcttctctggaacagaccccagctgctctccctgtctctgtgctggtctctcaatccatgctccacactgcagccagagtgctctacaatgcaaatccatttgtgagactcctcctcttaaaatcctcaagtggcttctctttgcccccaggatcattttgaaactccttaatggaagaggcatggccctttgggatgtggttccccaacccctcccacatcatcttttcaatcagatttcccactaaatggaaattttttcaggtcctcaactttatggtgactttctcttgctcaggatctttgaacatactgtttcttctttccttttgtatttgccaagacaacacttcctctggtaagattttcctgacatcctctataaaaaaagattgagatagttgactacccaaaatgtttcccattcattccaagctctattcaaggcagtaaagtgcccggctgacagattgcattcctcatcttttctgaagctagcaatggccatgcaacagcattctggccaataagatagaagtcgaagttgaagggtgggatttccaagaaagctcgttgaagacataattcctcatttcacttcttactctttctctttcctgcttcctaaaatgcggtgcagatggcagacacttcaaagctgtctcaggcaatcaggtgatgttaaggcagaaaccagctttatgatgggtagaacaggaagaaagaaggcacctatgttcttgttcaccttgaaccacaccagcactgccttgcctacccctggaattcctttaatgagaggcaaatgagagcttacgtgtttaagccattgctattttatttttttttgtttatatgcaaaagaacttaatcctaactgatattaacactaactgggtctattgcttggtaccaagccaatgcatgacacatggtatatatgctcagtaagtatttgttgaatgagtgaggcaatgaaagaacatagaggatatatataacagtcctcctgcccagatgtcatctgatcctctttaggatctgggcccataaaactgtatctgatatagtttgaatatttgttccctacaaatctcatgttgacattttatccctaatattggaggcagggcctagtaggaggtgttttggtcatagtgataaatggcttggtgccgttctcacagtaacgagtgagtttttattctagtggttcctgcaagaactgattgttaaaagagcttggatccttccacccctctctcactcttgcttcctctctctcaccttgtaatctctacaagctcttcacctccccttctccttttgccataagtggaagatttctgaggcctcaccagaagcagatgttggttccatgcttcttgtacagcctgcagaaccatgagccaaatcaacttcttttctttataattatccagtctcaggtattcctttatagcaacacaaatggactaagacagtttctaatgctatggttcctttagtaggtcagtgtaaaaccctggatcactcctgtaacaaattacttggaactcttctcaccatacatatttaaaaatagttgccatgttgaaaatcctataagatcatattttatttcaaatccaacaactcattgctaaggagatacaagaagcagaaaatacagagagactaatgtgttgatgatttttgtgagggacataaggtctgtgtctagattcatttttttgcatgtggatgtccagttgttccagcaccatttgttgaaaagactatctttgctccactgtattgctttttctcctttgtcatagatatctggtcaccttaccttagagtcacagatgaatggtcctattacttaactactgaaaatacaggccaaagcaaacagaggaataagggatatataataaagtatttgtgtacttgacttggctctaaaggaagcattgcgtgtctgtgtaaaaagaatgggtgagagttttccaccattcaatatttctaatctttctgaaatacaaagccaggacatcctctaatccatacattccatagtttggttaatataaattcctttattaaatccttattaaataaagttatttatgtttctatgaaactcattttaactcctaagtgaaaaatactactgagctaactaaacatcaaacatttttaattttttaaatttttttagagacagggtcttgctatgttgcccaggctggctttgaactcctgtgctcaagcgatcctccaaactcagcctcccgagtagctgggactacaggtgcatgccactgtgctcagctaaacatttttttgaaatgctcttttaaaatcaattttattgaagtataagttacataccataaaagtactcattttgagtgtacagattgacaagttctgacaaatgtgaacaaccatgtaaccatcaccaaaaataaagatatgagacatttccattaccccaaaaagttcccgtgtccctctccagtcaatatccagccctagccccagctccaggcaaccaccaatctgctttctgttgctataaattgtacttatcttttctagtgtttcatacaaatggaatcatacagcatttactcttttgtgtctgtcttcttctgctcagtgtaatgtttttgagattcatctatgttctgtgcctcagtagtttgttctttttattactggataattccattataagaatataccacaatttgtttatccatttactgcctgatgggcatttggttgtttccagctttgaactattttgaatcctaaaagactgccagttttgaatgagaccccagaacaatgaatgtaggctctgtatacaagttcaggctgctgggcaacttaggccttaagacacaactctgccacttaggccttaagacacaactgacatgatggtgcttaaagtggctgtgatggaaaaggaggctgtttggagcctttggagtgcctttataggtgaaccccagcatagcacctaatgatttggagcaaagctgtgtcattccccaaagataactattcgccttttgagaaacatcttctagctactatcaataataaacacagaatgcatcaccatgggccaccgtgttgtcttttgacctgagtttccattgtgaacaagagtcatttgatccaaggcagaaagttgggtgcacacagcagtgttccatcatcaaatggaatatgagattgggcccaagtaggtcctgcagacacaaataagttgcaagagcaagtagtacaggcgcttggcctggccagtactgttgccaagttgactgcttcccctcagtctgcatctgtggcttcatggggagtttcctatgaccacttgatggaggaaaaaacaaattggagcatagtttatagtgctggtactacccaaagtggctagctgaggcactacatctccactctggggtgcccgtgaaggacagtgccaaaggaaaaccccctcagtgagcagaacttggagcaatacaagtgggtgttcattttacctagaagagaagatgtccgtgagttacagatctacacaaaatcacagagagtggttaatcgtttagtctgatggtcagggacttccaagagacatgattagaaaactggtgacaaggagtcctggggaagaggcatatggatacctctgaacacacacaaaacatgagaatatgtatcccatatgaatgttaaccaaagagcagccacaacagaagaggattttaaaatcagctgaataagatgattcattctgacagcatcagctagtctctttccccagccactgttgcccagtgggcttacatatatcatggccatgggggcagggctatgtatggacacagcaacatgaatttccactcatcaaggccaatttggctccagccattgctgagtgctcagcctgccaagatagaaatctacgccaatatggcaccattccctgggctagaaaaccaactggtggaaggttgattacattggaccatttccatcatggaaggggcagtgctttgtcttccctggaatagacatttactctggatatggatgtgccttccctgactactacaatgctctgccaaacctaccatccatgggcttaattttatttgttataaaatttcaaccaccattgcttctgaccaaggaagtaatcttacagcaaaggaagtacagatatgagcttctgatcatgggcttcactggcctcacagtgaagcaggtggccagattagaacagtggaatggattttaaaggctcagttacagcaccagctgggtagcaacaccctgctggcctggggttatgtcctgcaggatgctttaagtcagtgaccaatatatgatgctatttctcccattgtcaggattcatgggtccaagaatcatggggtcaaaatgggagtggcttttctcactatcaccctggtgttcgggtagtaatttttccttcccattcctgtaactttgggctctgctattgcagaaatcttagctcctgtggggggaatgcttccatcagggaatacaatggtggttccactaaactgacagctgagtttgccatctcctcgtgccagtgaatacacaagcaaggaagggggttcctttctcacctagggtgactgatcctaattaccaaggagaaattggactgccacttcacaatgagggtgaggagtatgtactctatgtgtctgtgattaatgtcaatagaaagtgacaccaacctagtacacagaggactgatcatggtccaggcccttcaggaatgaagatttgagtcaccaggcaaggaacttggactcactgaggagggcatattccaaggagaatattttatctatgtccatctatgtccatctatattccatctgtgttccccttggaattcctattcatgaacatggggaattccaaggggaatatagaatgagtagtggaaggtagttataaatgtaagtcaaaaaccacacaaccaatttgagaaatgaggaaggtaatagtgttgaatatgtcttctttatcttgatataaatgtatttgtgcatatattaaccagtttatttatttattattattttttgagatgagctctcgccatgttgcccaggctggtcttgaactcctgggctcaactgattctaccatttagtcctccgagtagctgggactacaggcatgcaccaccatacccagctgaccagttttttcctattcctctacttaatttctctactatacaacataatatgtgttaatggtagttaactttatatctcagtattaagtcacaagatatcaaaaagggaatgcgacttagttacaagcagaatgaatatcactcaaagatgaataaagagaagagggttagtgcattttctgttggatgagagaaagtttcattgttaggcagaagcatgattttgccttttttttttttttccaaggtctcactctgtggcccaggctgcagtgcagtggtgcgatcttggctcactacaacctctgcctcccgggttcaagtgattctccagcctcagcctccagagtagctgggattataggtgcgccaggttaatttttgtatttttagtagagaaggtgtttctccatgttggccaggctggtcttgaactcctggcctcaagtgacccacctgctttgacctcccaaagtgctaggattacaggtgtgagccactgtgcacagtcaccacggtctttttgggaggcaactttagcatggttaagaggtgcgaatggatgttaagctaacaccaggtaagccctggtagatgtgtattgtgtcagtgggcctacgctggagccatgtttccccaaattcacttttcctatgtacctctggattagtgtgggccactggagacatttcacatgagatgaggaaggtgggagtgaaggagcagcatctttttacactaagcaggtcggggagggcatgtggctctgtctcacattgttgggaatctgtccatcatctggttggcttaggtcagtgggtgagttcacagctgttccagcttctgctggaaactccttcggtttctctgactgctccgtgatgagggcatcagattctcctgcagaaagccccagtgttgaagttggggcttcatgttggtgagtgatagttacgggttctagcccaacctgtggtttcttgcaaatttcagtgtcagctcagtcttgcgggttttgggttgtccttgcttcccacacttcatgcctttctttccctcctgacagtctgccctttagattttaggattcagcaccagccacagaaacagcaacctcactgttaagggttgaattgtatctccccaaaaggtaggttgaggccctacctgccaggacttcagaatgtaacctcatctgggaatagcatcattgcaaatataattaattaagatgagggcatactggctcaggatgggctcctaattcaatacaactaatgtccttctatgacagccacaggaagacagaaacgccaagggagaacaccatatgctgatggaggcagtggcagctgccagccaaggattataaccagaagtcaggaaaaagcaagaaggaatcctcccttagtgattttacagggagcatagccctgctgacaccttgattttggacttttattccccaaaactgtaaaacaatacacttctgttgttttaagccactcagtttgtgctactttgttatggcaactccagaaaacaaaaatacactcagactgtttaatcaacctccataattgcataaggtctaatccctataataaatcccttaaaaatgtctgtgtatatatatttaaaaatataaaatatcttctagtggttctgcatctctggtcaatccctgactgatacagaatatgtattttcatttctaatgatgaaatacctgaatgaaatttctaggacatatggtaagtgtatgtttagcttttaagaaactgccaacttgggggaattgcttgaggccaggagttcaaacagcctgggtaacagtgataccctgtctgtacaaaataaaaaatattagcagcgtgtggtggtgtgtgtctgtagtcccagctactcaggaggctgaggtgggagattcacctgagcccagatctttgaagttatagtgagctatgatcacgccactgcactctagcctgggtgacagagtgagaaagctggtctctaaaaaacaaacaaacaaaaaagaaactgtcaaactcttcccaacatgttgccatttttacatttaccattttacattcttaccagcaatgattgatagttccagttgctccatacccttgctgaccattccaatagatgtattgtgttatctcattgtagttctaatttgtatttccctagtgattaatgatgtttaacatcttttcatgcacctattggctatatgtatatcttctttagcaaaatatatgttgttatttgaagagcggaagttttacattttgatgaagtctaatttattgattttttttttcttagatggctcatgctttttgtgttatctaaaaaaaatttgccttcttcatggtcacaaagactttctcctatgttttcttttggaagctttatatttttagtttttatgtttatgtttaagacccatttctagttacaatttgtgtgattttttggaagggtcaaggttcattttcttttccataagaatgtacagttgttctagcacccttgttaaaaagactttcctttccccattgaactactttgtcaaaaatcaactgagcatatatgggcatcatgaattttaatcctgttagaactgaatgttcccaaggcaggccatgcccatgactgacctcctttccttggattgcctacaaaacagataaagctaagtctggagcaaagaaatccatgtctaacctgtatttttttttttttttttttagatggggtctcgctctgtcacccaggctggagtgcagtggcgtgatcccagctcactgcaatctctgcctcctgggttcaagtgattctcctgcctcagcctcccgaggggctgggattgtaggcgtgcaccactatgcccatctaatttttgtatttttagtagagatagggttttgccattttggccagactgtcttgaactcctgacctcaggtgatctgcctgcctcggcctcccacagttttgtgattataggcatgagccaccgtgcccggccttaacctttgttttcttacacaacacactacgtgatgttttccacatgcatgggtcatttgcttcatttacgtacaaatgcataagcaatatactgtgtggtgtgagtttgtgatgggaaaaggaagaagttttgcggatactacactggcttcctgctatctgtctgtgtgaatggctatggactttgtcttctatttgttcgcttagcgcagatatgatcagcttacaacttaagattctagagaaagagggtcatatctgtaaagcactctgagcatgtgtgaagtttaatcaatagcatatgaggttacagcaaattcactatctttgtttcttcagctatagaatggcatgaggattcatctcaatttagttcaattctgttcagaaccatgagctagctgttcatggaaggaaagcccacctgattgtggccagggaaggagaaacaacactttaaccaggttgatttggttctcacagacaccattggcatgtgacatctggaacagaccatgcctggtctctgttcgtatcacttactattcagctcaatattggtctgaatattctttagactgactgaaatgaaaaggaactgttgtgtaaccatccataattccagcctgtagacctgggctgtatctctatgccctgcctggcacagaccccacctcctgctccttctccctcaccaccagtcaatccttgtcctaatgaacagggagggcaaccctgaatggggagtggagggaagagatgtcatgagatggcaacgtgcaccctgaagtgaggatgaaggctatgtgaatgttgtaggctgacagccgggcatagtggccccgttgccatggcgatggaggcatgttgatgcgaagtgtctgcacagctcctaggatttttaacagcagctgggcagagcctcggcgtccctgaattgttgcccccctgagtcactgcttggccccagctgtcctgatctctgttgacaaatggttgtccttcacagtcaaactactaacagtactctaattaatgaatgtgctaattattcttgcctactcccagcatatttgtctaactaacctgtcacacacagatcagtgcagcatatgcataattacggagagcgctgggagcaggggatgggtgggagaggggtgggctcgcagccctgtcgctgtgggatatttcttgtaaagttacctttgctaacggtcagatgtcgtggggatatgttatttcccgtgaagtgtatatgtcttcctttctttcctttctaagaatctctcttcagggctgaggggccattgctcagtgctttagcctgtgaggggattgccaggtacaaatgcagaaggaccagggagcccaggttctgaagacgattccggtagcagcacgtagggtgattaaaactccagactttaaagccagaccggcctgggcttgaacccttgttctgctccttgctatgtgggtctttgccttgaccacattttttttttttttttaagacaggatctccctctcttgcccaggctgtaatgcagtgttgcgatcacagctcactgaagcctccatctctacagcctcaagcgatcctcctgcctcagccccgagtagctgggactacaggtctgtgccaccacgtccagctaatttacttttgtagagttgggggtcttgctatgttgcccaggctgttctccaactcctggactcaagccatcctctagcctcggccttccaaagtgctgggactataggcgtgagccacggtgccaggcccttgaccacatttttaacccctctgaacctcagtttcactttctgggcaatgggaggggggtaatttgtccctcagagggttgcactgaggggcaaatgtgaggctctgggtacaatgcccagtacagactaggtccccacgacacagccgctcagcggctccggattctgggctgctctggactgcggccaggcggtcttctgcgggaatccgggcaggcagggcgggctgcgctcccctccccggctctcccggtgccccttgtctttttgttctgtctcagcagctctctattaagatgaatggcatttccaaaggcttcacctctgataagtgttcctctgcagctgcagccagaatcttaatgtgcgcgctgtaatttaatggccgtctcggctattaacacgctcttctcgggtgaagtggactccctccatccccgggcctctgcacgtgctctgcgcgctggctgggggtgactccaaggagctcagagcggggtgcccggcacctctcgccaggcgcctttcgaccttctaaagcgcgaatggctggacttttctcccatgtgtggggccccagaaggtgtggggccccagaaggtgtggggtccctgcgttccacggagcccggaaggtttccagtgatggtgggggctgaccacgttggtccccgtgggtgctgttttcatgtgccggcagattgggatgagtttaaaagacagaagcgtgtaggatagagaaacttctttaaaaactggaaattttaatctggggattataactattggacagtcaagtgcaagagtgaatacacttctcactccctcctcccaatttttatttgcgggattagtcagtccccctctgccacatgataattgtgagaactaccagggtcttcattctcctgccatctggttgacctctccaagaatggacacccgggcagcctgggccaatgaggctgtcctaagagtttagatgagagaagtcagtctttgacaggtgatggaagctgtaaaatgtaaaactccacagttggtgaagatgtctccaggaaacaggtctgcagagagaatacgtttgacatgctaagagaagctgagagagagcgagaggagagattggaagaaagacagagacagaggtagagagaagggaaagagagagagaaagggacagaagagagagaaaaaagagggggccgggcgcggtggctcacgcctgtaatctcagcactttgggaggccgaggcgggcagatcacgaggtcaggagatcgagaccatcccggctaacacggtgaaacccccgtctctactaaaaaatataaaaaaaattagccaggcgtggtggtgggtgcctgtagtcccagctactgaggaggctgagacaggagaatggcgtgaacccgggaggcagagcttgcagtgagctgagatcgcgccactgcactccagcctgggcaacagagcaagactccgtctcaaaaaaaaaaaaaaaaaagagaggaagggcgggagagagagagagagaaagctctctagctccaaggcctaaccacatctctgttcttttcaacttcagctgtcagatttttagactctttgagtgaataaattctcctttttgcttaaactagtttgagctaagtttctattgcttgcaactggaatactttgtaagaggactggccttcatttctgatgcattgtcactaagatgtaagtgttagaagagctaacgctttatggggttcaaactccttggctaccaaaacctaaacatcccctgaaacttaccaaactgcaggtatgaattggatctcactaaggtgaatatacaaatcttgcaagtgctgagccctaaccaatcttgtaataactctgtggtagttaattttatgtcaaattgattgagctaaaaaatgcccaggtagctggtaaaatgtttttttctgggtgtgttagggagggtgtttctgaaagagatcagcactggaatcagcggactaagtaaagaattcccaccctcaccaatatggtgggtgtcatcaatccactgagggcctgaatagaacaaaaagcgggcagaagggcaaattccctcttcttcttgagctgggccatccatcttctcctgcccttggacactggagccccttgttctccagcttttggattcagactgggtcttgcaccattgccctccatcttctcctgcccttggacactggagccccttgttctccagcttttggattcagactgggtcttgcaccattgccctccttgatgctcaggcctttgaatgcagactggtctccaccagcagcttttctgagtctccagcttgcagatggcaaaccatgaaacttcatggtgtccatgagcatgtgaaccaatttctattataaatctgcaatatatatatatgaggagacttatttatatattggttcagtttctctggagagccttggctaatataaagtctatactctacaaagtgccctaggtactcagggagtacccaagtgtgtcatgaccagcccgacagccctggctgctggcttccccgcacacaactctgcacgctgccttcatcagcctttctctctcagctgaaccgagggcattgaagcgggcctctggcactgtacctatgagggagcaatatcttcccctacactgacctcttccgtgccgagatgcagccctccctgctgccactagttacagtggtccatgttccctttcaaagtgaagttttgataaaagcacctcttaaccaatgccaaatagctaagtctgggacaaagattgcaggtattttgcattttccatgtaacctcagagggattgccattcacactgatctgagctgcagaataccaggcagccacctcacccacccagcaggtccactcttatactttctcagaaagcacagccactctactcttattcagttgaaaagaatttccaggaaggtgtttctgcgattgcctcagaaaagtcagttccctttgggaatttcccttagggatcatctgtaactccatttctgccttttacctgaattctttggtttggtttgaattctttggtttaatttatgaattccctttattacttttctctgaagaaatggagatatcagctgtccctccccactgccatttattccttccttcattcaaaccttatgtggctgctacttaccgtgtgttaagtgttcactttttttcttggaattcaaaaaaagaaggacagtatttggggcacagatcttttggtgttctatacatttttttaaagtttcattttacatttgtgtgtgcgtgtgtgtgtgtgtgtgagacagtcttgctctgttgcccaggctggagtgcagtggcataatcattggctcactgtagcctcaaagtcctgggcccaagcaatcttcccacctcagccacccaaaatgctggggttacaggtttatgccactctgtctgacctgaaagttttgggtttactttcccttctttctctttgctgaagtcagagatgatggcagcttccagattctctggtgcctgtgctgggctcgtgctggtcatggtcttgggtccaggattcattctggagactctcagggaagtttcccatgacaaggaaatgtaggagagtgtgctggctttgcgtgctcctctgccaagccctgcttctcctggtgggacacactgaaccacagccagggcattttggtggttagttaaaaaaaaaaaaaaaaaaaaaaaaaggaagaagaaggcactgtgtaattgtgccggggatcttcagaaattgtaatgatgaaagagtgcaagctctcacttccccttcctgtacagggcaggttgtgcagctggaggcagagcagtcctctctggggagcctgaagcaaacatggatcaagaaactgtaggcaatgttgtcctgttggccatcgtcaccctcatcagcgtggtccagaatggtaaggaaagcccttcactcagggaagaacagaaggggagattttctttgatggttgtttggaagtcaggcttaaacaattgtgtctgtgtgtgcgcatgcacaaacacttttaccttatctttattttcttctttttatttgaatgtatagggttgtgtgtatttctgtgtaaatttggggttttcctcctcttagtctttcacttttgtggtgattaccagtcccatttttagagccagggctgcaacttgaaggttttgctaaaaccctcaccgaagtgtctatgatcagcattttaactattaattaatgtggccaggcaaggggtggaaggtgagaagactagaaagggaacatgatatacacatttactcagatactgggcttttctaacatctgcagtgcaattgaagttaccagtcatctgcagtctaaaaagaaagtgattttgggaggtgcgtagaaaaaatcatcttattatttttcctctatattacttttttctttttttctcctgaagaaacttttttttttggtgataccttctttttctctagcacgtataattttggaagcatttttcatatgcagtgtatacttcagaaagagagagagagagaggaaaattgtcctgttcagcgtttgcatttccattattcctgctattagttaaaaacaacaacaacaacaaaaaacaagcaggatacctagatctggaaaagggagaattgtgtagagctgtcttcctaaagttctgagttagggctgcctcagaccactttcataactatctccagtggctttgtgttttatatttattaagatagagaaaaaaagagtaattactaagggcagctgctgtagctttatggtgattactgaacattgacatgctgtcacgtttttggaactttgagtatttaatcactttgggatattctattttcccccatcttgagtgtggacagatgctggtgatgtagccttctgggcacagagcaagcctccccctcagcctctgcaccagaaaggctcagcttcacacactccaagtatgttttctacaagaactacactttgtggctttctgacccaaacatttttatactaaattacacacaacaaagttgtagctcagagagggaacaaatggcttatttaggccaccattttcttgagccattatgatttcacacagggctcccttggccctgtaaattggcaaggattccattattcaacccgcatacatgtacagagaccctgctctggcccagatagtattctgggtacaggcggatagagcaggaaacaaaacagctacagtgatggacaggtcagcctgcagcaatgcctgcagtctctgcaaaggtagctgtatgggtgggcaggtggctagcacttattcagctctggaaggatctcccctctggcctctcccctgacacccatcaataaaactgaggagcatcggtggacaggggaccttgtgccccctccctgcctgtgcagttggggctgaacccagctacgaagtttgagctcactctctccagctccctctcaattcagagctgaactgtgggaagcttcagagctctctgtttcaaggacaggttctcctcacctctcctaatggaggtgcaccagggaactggccctgctctgcccagggctttctcctggactttgccatcatggtctagcaaaccctgttcagattgaggtgagtggtgagatttcgaattctttttgacagataggattaagtcttcttctgtgggacaagtgggaggtagaggtaagattaaagatggccaaatgtctgagtcctgacagccacaatatggagatctagactttttacagaccacagggcacaggggcctcactaacagagttcccggaagtgatgagtgtgctgggggcttcctggttgaagagacactagaatggaccagctgggagctaattttttgggctggagtgtgatggcctgcacatcactgcctctgtccctccattgtcacagctgccccttaggagccagctgaggcaatttgtggtcagagtgactttgcacagttgtcctgcctgtgttcaggaagggagtttctgtggtccctttgaaaccacagaagagcccctcgtatagctctcaatggagggggcaaaacattcaaataactcaggagataacacaactatttgtttttaactgtgagtttttaggcaatcacaaagatccagatgtatgtccaagcctctctttgcaattctaattaacctcaatgttgcaaccatagacctaccttacagagttcaaaaaaatatgcaaaaaccctgcctttcttcttcctcataccccaaaatgccattctgaacatttcctgttagttaaaaaaagatttccatggtgttaccaggcactgtacacagtctgtgtcccaagacaaggaggtacagttccacatgcgcccatgactgggttgggctctgcactctctctatactttgagagcctgattttctgtgattgggcagagctggcccacctggtgcaatgtcctcctctgcctttcaaacatgttttagtcatcaagatcttcaaatttgtaaccctttccagcttgatccagcagaatgcagatttggaaaaacagaacgagtttaaaatacatgattctaagaaacctggaccagaactatcaaaacttggtttcccagagaatatagcaaatgggctcattggccaatactatgacattggcttttgagaaaagaaaggctttattgcaaggctggccagcaaggagacaggagttgggctcaaatctgtctccccagtttggggcttagggcaagttttaattacacagacgcatttcttatgagtagcaggcagagagcctccaacttcttctgcctaggtaccagcagcttagacatgatgcaaacctgggaagcacatactgtatttggagaaagtgattgggaagaaatgtgagctgaggggaggggctcagtgcccctgagctacacttagtgatggcagaggaaggatgtcctcccgcaggaggctgttccacatctgctctggttgtagggggagctggcaggcattagcagcggcctctttcccccaagagaggcagcctcctccaagttttggcgacattatggccctgcaatcataagggtttgtgagcatagtgctaaggagggaaatggagctgctgttactagttccaccccaacacacacacacacactcacaagaaacctcacaagcaccgtattggaagactttgccatccaacctgggatttgacaggctctagaagcagaatcatagactcatgaagttcccccaaagcaggaatcttccttacagtaacccccaaccacccccctccaccgcctccaccggctgcttcttcctgaacactgcagtgtttggaaaactcacaaacttccaagcttgcctttcctattgttgcatggattgaaagcttgcgttgtgtgaagaatggcgcttcctgctgtgcttagttttatctcatataatctttgcaccatttaatccttgcactcacccactcatgcaactgcctttgcagagactggaggggccgctgtaggctgacctttccttcactgtacctattttgttccctgctttattcccctgcacccaggacactgcctggcacaaagacaggtctttataagtgtatgcaagtgaataaagatatatatattattattgttatttttgagacagtttcactctgtcacccaggctggagtgcagtagcgcaatctcagctgactgcaacctctgcctcccaggctcaagtgattctcatgtctcagcctcctgagtagctaggactacaagcatgtgccaccacgcccagctaatttttgtatttttagtaaggacagggtttcaccatgttggccaggttggcctccaactcctgacctcaagtcatcctcctgcctcgacctcccaaagtgctgggattacaggcatgaaaccagcctagaaatacatactattatttattcttgttttacagataagcaaagtgagtcatggagaatttggttgaaagtcccaaggtcaggagtcgtgaagctgggattaaaacctaatcatctgactttagagagtagacacttgctccatgcatattgcctccaattcattcattcaagcactccctgctcaagaagttctttcttatgttgagctgaaatctgcagccctatgcgttttacccagcagtcctggtgctgttccctaaaatcacttagactgtgcctgctctttctgtgtttacagtgtcagctgtaatatccccctcttcggcctaacgtttctgaagtcccttgccactgggtctcctctcctcttcctgtgttctttctaagaacacctatgcagataggtgtcttctgtacagggaagctgttcctgagatccgggcatcgactctgttagaataatctacgtatgagttatttttttgagaactatgtgtcattgctgactcatattaactctgtggttaactaaaatctcaagatctctttatgtttgttgagaaacttatttaacttctctggccctccgtttccttcactgagcagtggagtgattgataacctccacctgtggttgctgaaggtcttgcacaagatgatatagttaaagtagctagcagtgcccacgtacggcggatgcctcacaacggtttgcagccatctctctatctgtgtctttgtctctctctcacactggttttggcttactgttagcagctagccgagataagtgtgtttatggtctttgcatgtattgtttctgtagcatactggaggattacaagaggttggggagtgagggggcggtgaggagtagacaaaggcagccaactcttccaagtttagcttagaaggaaggagcggtaaaccctagttgaatgttggactgaagcaggtttgtttttgttttgtttaaaggatagggaagatctgtgcgtgtttccaggataaagaaaaggagagaatatgatattaaagattctggaagtgggagaaggagcaatgaaatacagacttgaagtcagtggcatggacagggtcaagatcacagttagaggatgcagccttagagaaaaggaaggggctcggttctctgagcaaggagggaaagaagagaggcagatgcagagaagtacggcacatcgtgctgctggttgtagaaataacctctgacttttaataaagtcatccctcggtatccctgggggattagttctatgacctccctcggatgccaaaattcgtggatgctcaagtccctgatataaaatggcatagtatttgcatttaacctacacacatcctccatatcctttttttttttttttttttttttttttttttttttgtgagatggagtcttgctctgtcgccctggctggagtacagtggctcgatcttggctcactgcaagctccgcctcccgggttcatgccattctcctgcctcagcctacaggtgcctgccaccacgcccagctaattttttttttgtattttttagtagagacagggtttcaccatgttagccaggatggtctcgacacatcctccatatactttaagtaacctctagataatctctagattacttgttttgtcttttttttttttttttctttttgagatggagtttcactcttgtcacccaggctggagtgcaatggtgcaatctcagttcactgcaacctccgcctcctgggttcaagcaattctcctgtctcagcctcctgtgtagctaggattacaggcccctccccacccccaccccccaacaactggctaatttttgtatttttagtagagatggggtgtcaccacgttggcctggctggtcttgaactcctgacctcaggtgatctacccgcttcagcctcccaaagtgatgggattataggcatgagccactgtgtgtggcctagattacttataatacctgatagaatgtaaatgctatgtaaacagttgttatactgtattgttaaaagacagtaacaagaaaaaaaatctgtacatgttcagtccagacaaatggttttctgttttttttttttttttttaatatttttggtcagtggttggttgactccaggaatgcagaacccgcagatatagaaggttgattatgcgttcagaggcagggaataccatcttgggttccagaaagaaaatgatcagcattttctgtcatactctggtaaaaacagatcttttgaatggacaggtgtattaaaccctgtggagctggctgggcctggcggctcacgcctgtaatcccagcactttgggaggctgaggcaggtggatcacgaggtcaggagttcgagaccagcctggccaatatggtgaaaccccaactctactaaaaatacaaaaattagccgggcgtgatgacgcatgcctgtagtcccagctactcgggaggctgaggcagaagaatcgcttgaaccctggaggtggaggttgcagtgagccgagatcacgccactgcactccagcctgggcaacagagtgagactccgtatctaaaaaaaaaaaacaaaaacctgtggagctgatgaaatcctgcagggagcttcacggtgacagcaagaggagaaacacatccccatatgccccgcagagtttgaagtcccggctgcacctctccccagcagcaggttgactctggaaagttgcagcgttcttacctacagagtgggaacagtactacccattgcacagagtgggtgcaaagctctgtgacggaatacatggcaagtgcccaccacattgcctgggatgaggtgggcccttcctttacgtaagagagccctacagatacactcaaagtgggcacattcctacagaaggagtgttatttgtgtagaaaagaaaaacatgaaaggcttttattcctatacacaataaagcacccctttaatgtctttttgaggaggataatatgaaattgatgaaaaggaaccctgtggttggatccctgacaatcacatgtatcccttttttcactcttgaaaaaggagtaaaggaataaaatagaaggggagagggggcagagagaccttcaccgccccccccccaccccccatcatccaatctatagtcaaaccctccagactgtgtctccttggcatctctgacacccccaccgccaccaccccagtcaattcctatcttatccccctatcctggatctgattctgctaagttcctgccacactaaagacagggtggctttctgatgacaacattcctctgcttaaacctgtcagtaattccttgttgctctcagacggaactaagttctgaatttcttcacacggctctcagcaaggtcacagtcaccctgctaggccccaggggcaaatctcaatggtcatcttcttgaagacctggctcagttatttctttctcattgaggctcacgaccccaccttcttgcatgcctcaaacggccccttaccatgctcttctttcgcccatagctcagcacaccatatcattttaatttatgtattttgcttaatgtggatgatctgtctcctcctctgctgtcctcaccagagcatcagttcctcaaaccaaggctctttgttttgttcttggatgcaagctaaatgtctggcatgtggcaaatggtcatagatacatgtcattgaaagaatgattcatcacctccctctttggccttgtctgtggttctaccaaatcccattccctccccagtgccctccattccccctccttggctgaacattctgaaccacagacagttctttaccctgaacctttgcatattttgttctcttagcttagagcggcccctctccctccgtctgcttggctaatttctacttgttcttcagattttatcttagatgtcattccctcaaggaatccttctgtgactcaacatggaattaagttgcctcctttgaccctgaaagcaccatgtactcaatctcatcttggcatgactcactttgctgtgtggaatgtctgctttccttgtttgtctattcctttagactgtaagatcctagaaagtgggggccgtgccttgctcatgactgtgtttctaacaccaaacacagtgttcagtagagagcagctgctgagtacgtttctgctaaatgacagttgatggaggacatttagggttgcttggaggtcaagtcaaggaggcatttaacattctagtaaaacaaggaagtaacaggctcctgaacatgcccacaatgaaccagatgcaaaccttttcccttggcaggattctttgcccataaagtggagcacgaaagcaggacccagaatgggaggagcttccagaggaccggaacacttgcctttgagcgggtctacactgccaagtgagtcctaaccctgatgttgctaataagtgggggcatgggcaggggggcctccttctaggagtgatgaccacccttaataccacatgtctgtctgagccaagtttctgagcgccagggaggtgaggaaggttggacttcaccagagaggctttgtggacaccctttatcatcttagtgagtgctagtgtcaaaacaaagggagtggggatatggggcacattggtggagggaggtgtgatctctgcagcttcagaaagatctgaaagagtcatttggttagagaagttgacctatttcctgtggggttagaccagggttgctactgtgaacaccagccatgactcaccagtcaccttcagaagccacaggcaggacatgctgacgacagccttcaactcacccaccccttgctcccctgcgggtggaagtctggaggtgacaccactgcattttctaacacgggggctccttgagcaactagaacaagaacagaaagaatggggacattagcaggtgctttccccctctctcattcttttctttgaataaaaaggttgtttgaaaacacctgagcggctcctaaagatgggtgcaatctattcgggatgcaaatccgaatgaatgttattcaaatgctcctctcttctttatgcagagtgtatttcaaggctcagccagtggcaggcatgctggggactatggactacggactaggggcctgtcacagaggaaggcctcatgctagagagctaagggaggagctggccttcagttccatcccaggagcaactttgatgttcccagagatccttccaaagggggagtcatggtcacccaagaaaaatgtattcagaatgccaagaatggtgcaaactcaggacaaagattcacactgcagggttggagtccctgggcttgctgctggcaccatgggagggagggtccccttcaggggtaccgttggtttcctgtgaattaaactggcttcaagggatctcgactgaacaggcctatatcacactcactgatatactctctcttcagtccttctcctcatctaggtatttttaattgtttcagtgaggtgtaggcatgaggggattggagggggcatctcctccattgcagtttttcattggctgctttgctccctcagctccgaaatcgctgggccactctcgaacgcattagtacggtagtcacaggttgattgcctggccccttgccctctgtgggcattttccctttcagacagcccctgagtactcacagtgctgctacagtgggccacctagatctccctctttctccatgctcccacgtgctctgggctccactcccttctcccaagcacttctgtccagggctattccagcagtctgacctcaaggaaatcctttgctaaactgattatagagaggtttctattttaacatttaggtcttccatgtattaattctcagaatcaatttaagatgtttaaaggtgtgatttaagacattttaaaaccatttggaggagagtacagaaattatgtcacttgctgtcagcctctttgcaccatctgcagagaaagatactagagtcccgccttggacacatccacatgcaagaggtgcaaagaaggtgtctttgatgaggcaaggtcaaaacttctccccagacgaaatccaaagaaagcattcctactatgctatatcagtttggaaagaaaaacttctgccaggtgactgcattctcactggtcacattgtgttcctatggactcctcagctcaaccaatttggagaagttatggtgcaatttcaccatatctggttagaagttaagtttccaatttgctggcaatgaagaagaaatggagcaggccaggctgtgtagtttctgccacgtgcccccgggagtgaacagctctgtttgtaagaagccatggtgcttagacctgggctcgctagttgccagcctccaaattgcagaagtgccctttggttggtggctatgctgtgtcacttgggaaggtcgtttggaagttccacagtcgttgtggggtgccagagattaaaaagcgtaagaggagagtggaaagtgattgttgctgcttgggcatccccaccgtgtgggtgctgcagcccagctctcaaaacccatgggtctgtacactcaacctccatgagagggaaggagaaggatgagggaggggagagatagccatggaaaggtaggaactaagcaggcagggtggagagttttctgtaagacaaaaactgtctggacactgctgcggttctgttacaaagaccacttcctccctgggccagcaacatatctgtgtgcctgtctgggttgtaaaaagggtcaaagatcaatgcagcaggcagctacatgctggcaaaagccagaggcagctggtctgtttgcctgtgccaggaaaccactgggaatggggttgtgtgttattctaggagaaagtcgtcccagcagcagcttctccaggggcatccaagagcactgaaaagggttgcaagatgacccatgaggctgcaggaagaaaagaacatgcatttaatcttgctatctgaaaagtaagacatgaagctttcctcatttttaatatacacatggacagtagtatgtgtatatagtttatatgcaaatatacttgttataaggttgcatgctcaaaatttttggttcatggggtgtgggatcataaatgtttagggaccatggctatcaaggaaaaacagcatgaaggataaatgatactggtggattaaaaagacagatgcatgtatttttagcataaaacacaactgctgactgatacagatagctcaagattctggggcagctgctgaacagatacactagccagtgtggctcatcggctcagacttggccttaattaatgggctgtccctccacccatctcccatgagggcagagctgagccagggtttgagagctaaaaggaattggacctggactctgttcacgtgtatattttaattctaattaattcattcttttgaaagacagagtcacactctgttgcctaggctggagtgcagtggcacgatcttggctcactgcaacctcggcctcccaggttcaagttattctcctgcttcagcctcctgagtagctgggattataggcacatgcccccatgcctgactaatttttgtatttttagtagagacggggtttcaccatgtcaggctggtcttgaactcctgacctcaggttatccacccgccttggcccctcaaagtgttggaattacaggtgtgagccaccgtgcctggcctgttcacatgtataaaacacagtttaatgtcctattcccagccaatgagcatggctagagcagccttggtcaaagtttggtttttggagaaaaatccttgttagctgacctaagattcctctttgtgagtgtaagtaagcacaggttgcagagaggagaagggtctctggagaggtgtaattttctaaatggattacaagttcatggacttttaacaggtgttacaggggataacaagttctttatagacagacttttgaggacgtttaagggtattctgattcttggttttctaagaggggaatgtattatttaactacagacacccctaccgcccactttttgcagagtgtatcaaaacatgtttttggaataccaccctcatgtcgcttctccctgcatctcttatctcttggtgtccattctagactcactttctttctgttttttatttttatttttttttgagatggagcttcactctgtcaccaggctggagtgcagtggtgcaatcttggctgactgcaacctctgccttccgggcttaagcaatttttgtgcctcagcctcctgagtagctgggattacagcatgcaccaccatgtccggctaatttttgtatctttagtagagacagggtttcactatgctggccagcctggtctcaaactccttacctcaggtgatctgcccgcctcggcctcccagagtgctcagattacagacgtgagccactggtgcctggcctagactcactttcaagtggcatagacttgtaaaattatttaaaggtgataggtctacaatgatcctgtcaattagtattgacactattattaataaactgttattaattatatttacttactttaaattaatccaaactaattaacggaacactaaagagtttctatgttttattcccagaggtggagaaaaatgaaagggaatatagcaacgaattcttttctccataaaaacatgaatagtgcagcacatcaagttgaacataccacagcaaattgttgcaagatctgctgagtagctcctatttagacctcaaggaatgagactcaaaatgggttcatcagttctgttttgcagaaaaaatagcgcaaaatttctcaaaagaaaatccagaataataataatttgtcaataggaaagacatttccactgggggttaagaaggaagacattggaacaatgatagccaccacttattgaatgcttactgtgagccaggtggcacttcaccttgtttcattctcacaacagtctagggaagtaattactaatgtctccatccacctcttgtagatgagcaaactgaggctcattgaggctaggaaatgcacccacactcacatagcccataagaggcagccatggcattgggcccagaccatgtgaacttcaaagactacacgagcagccactgggcagctgtcatggctaaagccacttgaattcagcccagcagcaaccccctctccaggaggggcacataagcttgcagctttgggtagaagctgcacttgaagtcctggatggcgagagggactggcttgagccagagccaggaacaaggctctgagaatattctggaaatccacaggaggaacccattttcttacagctgggagaatttcattcaactccaggctgaccatgttttattaggaacgaaggtgacttgaactaatagtcaggaatggttgaatacggacccaatgtcaaatcactaggcagttcacatttctaatgagcaaatcccttagacaattaagaatttttttccttttgcataacccagacaaaatcgctacttaaaaacaaaccaaagacccgaaacatgagaaagagaaggaagcaggggaaatctttggtactaataagtttttaaacaataagagcaccagatattttaccccatcagacacagaatgttattcgaataaccaaaaaaggaattttttctctaagtttcttgaactggaaaatgaatcatattttctcagtcctgaggctgcaattttgtgcctctagtaacatataagaatagatgtgatgccagtgcccagtagctgctgcaattgttacttggggacctgtttattcactaagcacttcaccccagtgataaatttgtaggggcctcctgccctttggagctcctaccgtgtccattagatcagtggaaattctgggattcagagcactttgcaaggtcagcaggggtctgctctttctgtcctgttcctggtttttggttgtgcctggattccagggtaggtttctcatctgttaccttcatagacttctccagaaaaggatcttttgaccatcagaggaccacgaagattccattggtgaggcgcagataacctgatctctctgggttctctgcagggcacagatgaagggctggccattcccaagttctcagtggtaccactgaggcatgagaccctaatggtttgcatgagcagtttgaaaattgcatctttgtttttacctatataatcacatgaaacccgtggttctcaaacgtcagcaggcatcagcatcacatggagggcttgttaaaacagatttctgggccccaacacagagttttaaattctgaaggcctgaggtgggtgtgaacatttgcatttctaacatgttctcgatgctgctgccgcctctggtcccgagagcatgcctggagaactgccaccttcgaccatggactgtgagaattcacatggacctcagaattataatcagtctctcagttttacagataaggaaactaaatccagagagattgttttgccaatggtgaacagctggttaaagtcaggatggagactttaatcctagtcaagtgacctttcctctgtatttatttccctccctttttatgcctctcaagtctagttacactgtttttcatggatgggcatatttattgtcctgatctggactgcagacttctcaggaggacacctatgatttaatttagtatagttgaagagttaacagacatggctttggagacagactgattatggtgtgaatcccggctttgccactccctagctggatgaccctgagcaagttattcagcttctccaagcctgagttccttattggaaacatgagagcaattgtgataggcagaataatggccccctcaccaatcatgcccacatcctaatcctaggaacctgtgaatatgttatgttacatggcaaggggaaattcaggcagctagccagttggccttaaaataaagagattatcctggatgatctgggtaggacctgatgtaaccacaagggtctttttaatgtggaagaaggaggcataagagtagatgtcagagtcattcaaaataagaaagatttgatgggccatccctgactttcaggttggaaggaggttctgagtcaaggaatacaggtgacctctagaagctggagaaggcaaggaaatggtttctcccctagaagttccagaaggattgcagccctgctaatatcttgactttatagccctttgagatttattttggatttctgacatcctgaaccatagtaaaagggtgttttttgtttttttgagacagagtcttgctctgttgcctgggctggagtgcagtggtgtgatcttggctcgctgcaacctccgcctcccaggttcaagtgattctcctgcctcagcctcctgagtagctgggattacaggtgcttgccaccacacctggctattttttgtgtttttagtagagacagggtttcaccatgttggccaggctggtcttgaactcctgaccttgtgatctgcctgcctcagcctcccaaattgctgggattacaaggcgtgttgttttaagccactcagtttgtggccacttgttacagcagcaagaggaaactcatacagttatcatgtgaactcacaggaatatggtgagttaaaaagagaggaagggtgcaaaacatccacggtagagtgagaactctccagggagtgaggactgtgcccagcatacagtgatcaccctcttagtaagctaagtttctgagcaccagcttttttgagttgactttgttgtctttaacatttgaagatcacccttctttgctcagcctggcttgcagacctgggctgatttgtggatctgatagaaaagtttccttagttgggctcttctccccgaccacccccatgccagtgtggccacatcctctgtctgcattgctcactcttcaattccaagaagcgcaggggcaccgccaggaacaggaaccctgccagaggaatacatcaagaaaccaagtctcccttacgcatcaccgtaggaacagagttaatggattatgaacatgtgtttgctttataccattgtttgtttcccaggtggcagctggctgccccatcttattgggtagatgtaagtggaattacgaatgggatttatgtttcatgcacgatggtgattattaacttcaactttcaggtaattttcagaccacattgcactaacttggtctctgattgtttttctccttgtttgtttattctgcagccagaactgtgtagatgcgtaccccactttcctcgctgtgctctggtctgcggggctactttgcagccaaggtaactcagacttccctttgttcattctccttctataaagtgcatctcaaggaggttcaaagggcaggctttttgttgaaaggactttgcctgacctctggctcccatctgtgaagccctggagaggtgagagccctcgggaggccgtgtttcaggcatgctctgcacccgtgcagagcgcgtgtgataatgcattgctaatgcttgctccctggtggctggctgagagctgctgtgctgacaagggtggtttaaggctaaatgtgactcagaatccttaagcagtgttagttcagatacaagggcattataaatgagagtgcctgagggatctattttgggaccgctgtcacttggctcttctgctaataagcttccagtgtggtggccctccttcaggcatgtttccactgagccacgggctggatgccacatccccggccttcccacagttatcagcagcccacaggcttgacttgagcaagttggaaagacaaatcaacttccagagttgatttaacattgagtggaaatcagtcatacttttggtcccctttcggggccacgcctggcactgtgcctggtggcagatcggcatgaactggccagcttctgtggccctggagggcacaggcagaaaggccacactcagtcccatgatgaactgtttaagacttattgttgtctccccgctctgtaaagtagatagagtggattttatgtcccttattacctttcaggatactttgactcagggagataaagtaacttgggtacagctactcagctggtgaagaacacaggcagaatgagtgcctgggtcttttgacttaaaattctggatttttcacaaagatcctcttactttattcatttacataataaatatatattgaagagctactctgtgccaagccctgtgcctagatatacagtgataaataaagagtagcttctagaggtcacctggcggtgaggcacaggccagctggcaagatggaccacagaagtcagtgaatgaagacaatgacaagggtgggaagcgccatatgggaagagaaccaagttcagtgatagagagcagaggtgaggcggcagcagaaaccacttaagggacaccacgtggcactccttctgtgctgagaaggctgtcagtaagctcaccatttatttcctattttctctcctgagttaaataggaaacatgtctcgcattacttgaaaaatcaagtcaaactatgctcttactaggagttatggttctttttatgtcttagatgatgcttgatctagatgaatgcggacttgctgtagctagataaatacaatgggagtttgaaggtgtttcgtagccctggaaataggtatttcctgtcaaaacaagctttgtcattgccagcagacaaaagcatcagtaaccttggttgataatcgtcatttcttaggaataaagtagactgtagaattttttttagcagaaaggaaacccaaagataattctagtgcaaatccctcactttatagagcagaagctcaagtcccagaggaacaagtggcttgaacgaacatcagaattttaggggctggatttgtaccctcctggtgccagcagcccacttccctgcaggaggcactcaccttccttgcacaggggtatgagtgtggccattttccacccataatctctgttagctcatgttcaattgggttcccattgaaagaaaaatggaccagtaagttggagcagaatcattcagatggtataacataaggaaaaactttgcccaaggcaaatcgtgattgtgacagctttgtgatttttagagaatagcatgggccaggcacagtggctcatgcctgtaatcccagcactttgggaggccgaggcaggcaggtcacttgaggttgggagttcgacaacagcctgaccaacatggagaaaccctgtctctactaaaaatacaaaattagctgggcgtggtggtgcatgcctgtaatgccagctactcgggaggctgaggcaggagaatcacttaaacctgggaggcggaggttgcggtgaaccaagatagcaccattgcactccagcctgggcaacaagagtgaaactccgtctcaaaaagagttcacagtttctcttttgctttgattttcttatctgccggataacaatagtattttggaaggcaggaggaattgtggaaagaaatgggttttggggagtggctgattggaggcaaatccaaggacactcattgctggtgtgtgactccaggcagttactcagcttttccaagcctcagtttccttattgtaaaacaggaccatggtctagctagtagcattcctatggtgagtgaaataatatgtataaagctcctgacacagtgcttggcatatatcagattgagccatgtaaaactgccaatatctggctatttatgacctacaaaaatagcatttcatatgattccacctaacatctgaagcgcaataaatgttattattgataatgcaggtggtggtgataaagttttgaaatcagaaagacctggcttcaaattccacgccttcactggcctgacttattttcattcatttgacaaatattattttgaacacccctatgtgccaggcactatgccaggctcagagatgatctaggaaaaagacagatgtcctcatctgtcttaggctcttgtggcctaagcctaaatttcctcgtctgtcaaatggtgacagtaacacactccttaccagagagctgggaggattggagactcaagttcccaaaacgccaggagcactgcggcaggtgaaaagtattccctcaatggcggaagtgtttaaattgcttttatatctgtagctctagataacactagttccagcttagttaactcccagctccaagccttcaggacttcatagagttattggggtgctgctcttggcagtttcccaaaaagctagaatgcagagggaatctccttcccaaaaagctagaatgcagagggaatctccttcccaaaaggctagaacgcagagggaatctccttcccaaaaggctagaacgcagagggaatctccttcccaaaaggctagaatgcagagggaatgtccttctcttctaaatggtagctgttagttcaagaaaggttaaacattgtgctgtggggaggctcaggggtgaagggtgtacttttaagagaaccagtttcagagctgggtttggggtttaagccctaccctctgcccccttttacgagctgacagccttatgcaagcctggttgaccacctgaacccacgtttccacatctggaaatagaaatgtgggtactagttatgttgaaaggactcaggttagatgatagatatgcaaataccttggaaaccaggagtgtccagtcttttgggttccctgagccacactggaagaagagttgtcttgggccacacatagaatacactaaccctatcaatagctgatgagctaaagaaaaaacgttgcaaaaaaaatctcatatttttaagaaagtttatgaatttgtgttgggctgtattcaaagccatcctgggccacgtgcgacccgcaggctccgggttggacaagtttgttgtaaacaatgccatgatgccggcataaggtcgttaccagtattaggaaggttctcaggtttcctctagcccttgggctcttttcctgaagtgcgtgtgtcttctgctagattttgtgaccaatgttgattgcctaattgggctaacagcatgttttggtggctacgaaactgacacaggtgttttcatttctccacttagttcctgctgcgtttgctggactgatgtacttgtttgtgaggcaaaagtactttgtcggttacctaggagagagaacgcagaggtaggtaactgggactactaaagaactgtggagcgattcctgatttttgagcaggaagagtgacaattcaaaacagtatttgactagattcacggctccgtagcatccccttgggtgggagggggaaggctgactaggacctctgattcttctttccctgagctttgaaggctctgaaaatacagctggggggacttgcccagttttcttattaagcaattcctccgcatggtgctggctttcaaagggtgcttcagtgctgtttgctgcacgtgccttgcagccccacaccctgcactcccgccctgcagagtctggcgctggaatgacattttaggtctgggttcccaggcctcctgagagtgaaatgtttcattgtttgtctagagaaatgagaactaaagcttgcaccttgtgataagttgtcctgaggaacatatctttcagggaccagaagaaagaatgttgggaaaataagatgcagtaagatgcagacatgacagcagggtgcagcggctcacgcctataatcccagcactttgggaggctgaggtgggtggatcacctgaggtcaggagtttgagaccagcctggccaacatggtgaaaccccgtctctactaaaaaatatacaaaacattagccaggcatggtggtgggcgcctgtaatcccagctactccataggctgaggctggagaatcgcttgaacccaggaggcagaggttgcagtgagccgagattgcgccactgcactccagcctgggcaacaaaagcaaaactccatctcaaaaaaaaaaaaaaaaaaaaaaaaaaagatgcagacacgagactgtgaaactgactagcatcaccattgcattgtttatagatgttgccagacagaaagccccaaagcagcacagtaccttcctgacatctggactaggaaatctagattttagtaaaatacatgctaatacttacagaagaaatgtcggcgttagagtatgccgtcagttccttagagattgcaattcctaatgcactagtatggtttcaggtgccaggaacacgttctgtgaggctgctgccccaggtgctgaccccagccttccacaccattttccttccttgtgttcacagccgctctgtcttttacaatagcacccctctctagtggctaatgggctctatgattagatagcatccttcagtagtgataaaggcagtgacatcctagggaggtcagcgggtgaaagcgctatatctggaaaacctgagagcctgtgaagctcaaggacttgacggggttagaccgtgagccgggctgcagctggaaaaagaatgactgttctttcagcagatccttccctgtgccatctctttcttcattcctctctagtggcattcttatttatcctctaaaaccacaattccattatctctcctattcttatcaacactgccctaaatgatattctttattctcttttgccctggaaaacctctatcatgccttttcccatgtgattacctcgttaagagtgggggtggaatgtctagcaatgaaataagagggtcttctcttttgcctggctccctatgcagccctatcttaccccctgcaaagtcccagggatgtggctcagtcactgctcctctcttcatctgtcaccacttgcttgagatcctacagctgctttaattccgagaccatctgcagaacatgacaaaatttgtccacctacccacatgtccttttaactttaaaggctttactaactgattcctattagggaatgaacagaggtggcaaaaataaacaataggagattgatttacaagaaatctttaaaatagtagatttcttcggacctcattgaaatataaatggcctgccttcttgtgtccctccctggtctccctctttaggtgataagaagaagatcctgccagccccataacccgccatctgcgcgggttctagacccccttctcctcccctctggccgtggtaggcattactgatgaatcatggtgctctttcttccagagaccaaacctggcctcggaatccttcttaacacagatactgcttaacacaaccactctgagcagctgtcataagtagaagtaatagatactagaagaaatgtctaagcctaatctagaccaaaatacggcctgatatagatgcaagccagaggggctttatggttaaatgcaaggagattttcaaccctgccgtctagaagctacttgctgagatcttcttcagttgggcccatctcctccccaggcctctcttctgttcctgggctatgtcacacttggactctgcagacacctaatgctcttgggacctgctttagttcttgacctcaccaaccgaggaggaattgctagatgagatccttcccccggaatttctctcttgaaccccagatggtccgttgcccctttccagaagttgctccagccctgtccgcttaggaagttcagtgtcatccttgatccagtgggtagggaagacattccataatgaatgccccagtctgagcttcttccttcaggcttcaggctgccctgcgaggattttgcagctccctttttaatgccctctagaagtttctggctcttattttcagcccttcatcctactctctctgaccccttcctctatcctgtttagttcacctgtagcagttactacccagcagtgaaggatgaatcttggtttcgtttcttttctcttcttttcttttttctcttctcttttccccttcccttcccttccctcccttcacatcacctcatctcacctcaccttacatagtcttgctctgtcacccaaactggagtgcagtggcctgatcttggctcactgcaacctccacctcttcccaggttcaagtgattcttatacctcagcctcttgagtagctgagactacaggtgtgcactaccacacccagctaattttttgtatttttagtagagatagggtttagctatgttggccaggctggtctcgaactgctgaactcaagcaatctgccatccccggcctcccaaagtactgggagtataggcataagccacccatgatgcccagcctgaatcttggtttcttccccattcatttaagctattacctgggcctgaactcaatggcacctggcaccaactggcaactgactcttggtcttttattacctaccttccctagcaggcactgggttgctccctcttcctatcccatggagtcctgtcctctgttggggctcctactgatcctcttggcaatatgaagttctcagctcaatggtgggtgggcaatgactgccaactcttgaggccaatgaactcaggttaccccactcctcctcctcctgagttgctcactcactcctcattcactcaacattgattcagtagatatttgctacctgctctgtgccaggtaccaggtcagttgctgaaggagtaacagtgaacatgacggagtctttgtccccaaggagacccaaggtgtctcctagagccaggggcacattgcaagaccaaatatattcaacttaccaaaataatcatagacctagttctcaaaaagcaagaagactgattcctcgttgtcatttctcctcctcagcatcaatgttttagagtctgtgggcccctccaagtgtggagtatggtgttacttcaccagagtttgaggagaaacattcttcttttggaaggccggggagcatagatggatatcaaggctgctgtttctaaaagcgaaacccaccaaacaacagtattagaatcatctgtggtgcttattaaagatacagattcctgggccccatcccagacttatgaatcagaatctctgccagaggaagcctgagaatttgcattctcagatgattctgcattctcagataacacattctttaggtgattcttacacacactggagtttgggaatcgctgaaggctgttcacttctcttttctgagaaatgattcattcatttcagaaatatttgcagaggtccttatttattggagatttgtgggtgggcagaggagaaatatcttgtcctcacagagcttacaatttttattttctttagaggtcaccaggcttaaaatgacacttccctaaattctgaaaagaacagatttttaaaacaagaagggactgtaatgttttctgttcctacctcgtattttgttcacattaagaacctggggtgggaagtggaggagggggggtgactggcggggggccacagagagctgagctggggtggtctcgaactcctgaactcaagcaatctgccagcctcagtctcccaaagtgctgggattataggcatgagccacccacgatgcctgggtggaactcagggctctggatgcctgggcgcccccatctcccacactacggcgcctcatcctagaagtggttagcacctttgagatgggaattatttagcaggatgcttttgtgttttcatgtaagttttatgctgcctgtggagggcacagctgtttcaaaactaataaccaaatcctggtctccgaagtctgaaggcatcctttgccctgcagtgcaaagcacgggattctggcctcacacaggcaggtctgaactcctgtgttgcctcttgctggctgtgggacctgaggcaaatcatgcaacctctcttttctgtttgcctagatggaaaataggtttacaatacgcccccataggatggctgtgagaattaaaggaagtcatgggtgtacaatacctggccccgaaagatgcttaataatttaattctgaccttcctcactcatttaggattatgtaccaacttttagaaacaatgaaagattagtgagtcttctgtggttggtataaaaaaaaaatagaaacatgaaagagatgtcctccttgttcaagggctaatgaccctggtgtgcgctgtctaggcccccaaggtcttccttccctgctcacagcatttcaggttctccgcagctttgctgagcctgggtcaggttcggtatctgcccaccatgctcacttgccacagctgtggccccatttccaaacttcagagacttaaaggtgcagctaatgatgtgcccggcctggggtcacattccctgagccctgcagacaagggagcaggaggctgagctcttatcttccacaccctgtgcacagcctgggaagagttaaagcaccctagtcctatgctgcgagggccacatgccctgagaccttggaaaaaatcctacctgaattgaagagcatcactatttcatcaggaggcgctgccatttcatttttcacttcggttttatcttgagtgtaaaacagcttcgcaaatcactttttcttgtttctgtaatgagcatatggtggcctcattcgtgtgataaatctgagccaccacgatatttgacttttcacaatttaatttatctgaaccctctattctctggctaaaaaatatcccttacttggacttctttattttattttcaattcccttaccagcactagcaggggactctgtactcatctgctggcgctgccataacaaagcactgcagcctggggggctcaaaccacagaatttattctctcacagtcctagaggctagaagtccaagatcaaagtgtgggcagggtcggtttctcctgcagcctctctccttggcttatagagtgccaccttctacctgtgtcttcacatcatcacctcactgagcatgtctgtgtccaaatctccccttcttataagaccccagtcatactggatgaggatccacccatatgagttcattttaccttaattatctctttaaacaccctgtctccaaatacagtcccattctgaggaactgagagtaaagattcaacatatgaattttggaagggacctaattcagcccacaacaccctcttttgggatgtttattttcccccttaaggagctagttaggatgtcttatctcatgaacatgactgtgaacaggaaaacagggagagaatgaagctggccaaggaacagggctggtgtcagctagcagtgcttttctgatgtgagtgggtcccacagggagcttgttaaaatgcagattctgattcattaggttccagagggacctgagatttcccatttctgacaagtttccagtgtgggggctgatgctgctggtccacggaccatactttgagtagcaaggagcttgatacataatggctgagtgactttcagactcctgctgtagaaaaattatgagttggctgggcgtggtggctcacgcctgtaatcccagcactttgggaggccgaggtgggcagatcacctgaggtcaggagttcgagaccagcctggccaacatggtgaaacaccatctctaccaaaaatacaaaaattagccaggtgtggtggcaggtgcctgtaatcccagctactcaggaggctgaggcaggagaatcgcttgaacccgggaggcagaggttgcagtgatctgagatcgtgccactgcactccagctgggcaatagagcttgactcagtctcaaaaaaaaaaaaagaaaagaaaaagaaaaattatgagttatattatcagcatatggggtgcctttcaaattgataaaatttctaatattaaacctgtggatgccaaatgctgctctctgattatggcaggaaacggcacttggcagtacgaagttagctgttgggctgagctggctcatcttgttgtgcggtcctgattgcctaaagatgccttcccaggatctttactaacaatcctcctgagtcatttggactttcccaacctgttatcacctctcagatgggccagccatggaggcagtcagaggagggctctgcagagggagggcagaaacagggtggcctctgcatgccattaggaggtcacatctcactgggggatgcagtttaggatttagtgccttggagagaaggatagagtatattaaaacatgtctccgctaggcatggtggtttacgcctataatcccagcactttgggaggccgaggtgagtggattgcctgagctcaggagttcaagaccagcctggctaacatgacgaaacctcatctctactaaaatacaaaaagttagctgggagtggtggcgtgcgcctgtagttgcagctacttgggaggctgaggcatgagaatcacttaagcccagaagactgaggttgcagtgagccgagattgcaccactgcactccagcttgggctacagagtgagactctatctcaaaaacaaagaaacaaacaacaacaataacaacaaaaaccaagtctctccctccactcaaaaatgcaagggcctgtctcccattgctgggtgcccaggtctcatgaatgtagatatgaattattccagtcagcctcaggagaatagaatgagccctcagatgccgaagcacctttcagattccaccggttttatcggctcatttaaacttcacttctaacacagtcctgcattacacacgtgtctgtcgttatgggcagctgcagagagggtcttaatggtcctaatgctcagtgaggatgcccaatggtcaacagaacctgccatcttcaggccatcaaggagctctggagttaaggaaatcatgagagcacagaggggcgggtacagcagagccctcgtggtaatgggttttgaggtctaggctctcttcacttgggtttgaaataagttcaatgactagtaatagctgagacacttctacccttcaaatgaagtaaatgggaaaatggagcattgttgagtccagggagctataatttaaaccccatatatctaaaaggggtaacatttttgtgtgtgtgaaattggtgtcattcgcactgcatctacagttttctttttccttctcttccagcacccctggctacatatttgggaaacgcatcatactcttcctgttcctcatgtccgttgctggcatattcaactattacctcatcttctttttcggaagtgactttgaaaactacataaagacgatctccaccaccatctcccctctacttctcattccctaactctctgctgaatatggggttggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttcttcacttatcctgttctctgaagatgttttgtgaccaggtttgtgttttcttaaaataaaatgcagagacatgttttaagctgatagttgaggggttttgttaatggcttttgggggatttatctctatacccacaaacgactagtttgttttcctcaaactaaatgataatattaaaaatacacatcctggccaggtgtggtggctcatacctgtaatcccagcactttgggaggccgaggcaggtggatcacttgaggtcaggaattaagaccagcctggccaatatggtgaaagcctgtctgtactaaaaatacaaaaattagccaggtatgctggtggatgcttataatcccagctacttgggaggttgaggcaggagaattgcttgaacccgggaggtagaggttgcagtgagccaagatcatgccactgcactccagcttgggcaacagagtgagactccatctcaaattaaaaaaaatacacatctggcttctggaaaaattacttgaagatcttttatgacatccatccctcttcacacagccatgtgaattaggttggtatcttcatatactagcatcgtgcccagcacttccatgttatacagtttaaaatgttctgtaattccctgtgggaacctaagataatgcgaggaccgtcatacgtgcccccaaatattggcaaaccaatgaataaatgaatgaatgagtttatgaatcgctaactggctgtatttaatgaagtatgtgtgttgagccatttcccacagtgtggacagatttgtcccacaatatgggcctcttcccaaaggccctaccacctaatgccatcacactggggatttgatttcaacatgtgaatttggggagagtgcaaacactcagaccatagcaccatctcagtaaatgtcccactggtcactcagttcatagtgacagtgatccagccactgtcatgacaggtgccacttggcagaaacagcacagcttggaagatggcggggtgtagtcaagattccaggatccccaacagagaagccagctcttataggggagccattcatcaggattgaactctcaatcgagctggacagtaataggtgggtctgtgttattccccagatgagtatcatgacagtcacaatcctaggaaggatgtgaagcctcccccagctctcctccagttgcctgcttgggcagcagagatgatggaatgtggagtctggcgtggtctgaggcctgaatccatgtgcctcatgtatgatgctcaggcaagaggatctctcaattcaagggagagggcctgaatgagccttgctttccaggcctgtctgatggtccaggctgaagcccctcctggcttgcactgccagacctcatccagcaggagctccttggcattgactgcttcaggatagttgcttctgctctgagtgctctctaaagagcagtgctctaccatccaagctgggcttttcttttcttcttgctgatagggaaggcatgggacattgcaggatggaagtggcccccaggccttctcatgcctgggcttggtttggaaggtggtcaggtgatcaataatcctgattggcctggcattgaggagttttcctgggatgtggtcctttcggttttttaaaaattatttttattgatacacatatttgtaggtatttgtggggtgcatgtgatactttattatgtgtgtggattgtgtaatgatgaagtcagggcatttagggtcttcatcaccttgattatcatttctatgtgttgagaacatttcaagttctcagttccagctattttgaaatagacagtccattttgttagctacagtcacccaacccggctgtcagacattggaacttactcctattgaactgtgtatttgtacccattcaccaaactctctttgggctttcagttttacaactgggatgatcctgggaaaactaaagtaaatcagacacccgacgtgtgagctaggttataatatgcccagtggaccctggggacatcttagctttcagaggtcatgctgtccaagctgactgtggggcttccagaaggtggggagaggaaatgatgcaatggcccatcagaggcactacttggggcctggggccagagtgcatgtctaaggcattaaggggaggggagagcagccttcataattatgaagaggagtctcaggtgcacagcttctgatgagggacagcttctaattgaagacagcattgtgtaatgctcaaactccctgtcttcagagtgcctgctgtatcccaccatcagttctgtgacttctccctaagcctcaattttgcatgtgttacattgggataataatagtgccaaactcatggggttgtgaggaataatgaggtaaagcaattgaaaaggtttagcacaatataagtgctcaataaaagccattattattattttattacactagttttcaattcctgcatagcaaattcttgcaaatgtagggactcaaaacaatataaatttattatctgacagtttttctgggtcagaggtcttactaggctgtaatcagagggcaaccaaagctgtgatctcagctgaagctcaggattctcttccaagctcactggttgttggcagaattcagttctttccagttggaagactaaagcctacagtcttcagtctctagaagccttttctctggcacaggtttctctacaacatggccatttatgtctttaaggccaataggagaacatgattagcatattttttttaagtgaactttagacccttttttaaaggcctatctgattaggccaggcccaagtgagctttaagtcaactgattagagatcttaattacatctgcaaagtcccttcatgtttaccgtataacataacttagtgaaaggagtgaaattgcaaccaggttctgcctgcactccacggaaggggattctgcagaagtgtgggtcacgggggggttattttgggattctgcctacgtcactgagtcaaaagaagctgaatggttgtgatgctgaggtttttgggcagcagcagtgtgtgtgtgtgagtgaattcatacgtatgaccacctgggaagaaaggaggctgtggtttcctccacctcctggcagacagagaaatttctttttttttttgagacagggtctggctctgttacccaggctggagtgcagtggcttgatctctgctcactggctcactgcagcctctgcctcccaggttcaagtaattcttgtgcctcaactccaagtagctgggattacagacacacactgccacgcctggctaatttttgtatttttagtagagacgaggttttgccatgttggccaggctggtcttgaactcctgacctcaagtgatccgcccacctcagcctcccaaagtgctgggattacagacgtgagccaccattaaccatttttctatctcctgtgggaaagggcacagtgaaagaacagatgaagctgagacatacaagtgaactcctccctcctctccatttagactaaaataggattattcatactgagattctccctggttgcaaagagataatctgtgcaactgggtttttacaattatccctaccctatgctttcctcatctgtcttcctcgtagtcagctcaggctgctataacaaaacaccataactgggggcttttgaacaacaaaactttacttctcacagttctagaggctggaaatccaagatcaagtttctggcagattcggtgtctaatgaggtcctgctttccagtttatagacagtgccttatcgctaccgccttacacagtggaaggagaggacgagaagctccttgggcttttttttgtttctttctttctctctctctctcttttttttttttttaataaggtcactatcttagtccattttgtgttgctaaaaggaacatctgaggttgagtaatttattttattttaaaaagtggccaggcatggaggcttatcctgtaaccctaatcctttaggaggccaaaacagcaggattgtttgaggccaggagttcaagaccagcctaggcaagatagtgagaccccatctaccccatctctactaaaattttaaaaaattagctgtgtgttgtaaagtgtgcttgtagtcccggccacttgagaggctgaggtgggtggagttcaaggctgcagtgagttatgattgagccactgcactccaacccgggtaacggggcaagaccttgtctctatttaaaaaaaaaaaatctttatgtggctcactattctgggtggctggaaagttcaagattgggcatctgcatctggtgacagcctcatgtcgcttccagtcatgggggaagacgaaggagagctggcacgtgcagatatcacgtgttgagggcagaagcgagagagagaggggagagatgccaggctctttttaacaaccagcactggggaaactaatagagtgagagctcactgactcctgagggaggacattaatctattgatgagcgacctgcctccatgacccaaacacctccaacgataccccacctccaacactgccacactagggattaactttcaacttgagatttagaggggggaaacttacaaactatcgcaggcactaataccactcatgagggctccaccttcatgacctaatcacttcctaaaggccttacctcttaatctcatcacattgaggattcgatttcaacttgaattttggggggacaccaacattcaggccatagcatcatctcaataactgtcccattggtggtcactcaggccccaaacaaaggaaccttcctccattcctttccgccctcccacccacagtcaatcatccccaagctccatcagctccacctttaacggccaacccacctctgccacatctcaccatctccactgctatccctgtcacctgggcccaccattctctctcctggacagtctccatagccacctctgtcagatttattttatttttttatttttttttttgagacaggttcctgctctgttgcccagactggagtgccatggcatgatcacatctcactgcggcctccatcacctgggctcaagcaatcctcccatctcagcctcccaagtagctgggactactggcaccaccatacctggctaattttttgttgttgttgtttaatttttaatacagatgaagcctcactatgttgcccaggctgctcttgaactcctgggctcaagtgatcctccggccttggcctcccaaagtgctgggattacaggcatgagccaccgtgcccagcccatcagatgttaatgctacacgcacttgcttaaaatcccccagataattctcgctgctcttggaataattcccacacaccttggcgtggccatgcaggctctgtgccatcggatatgtccctgccccctctcccaactcctcctttcgcttgctcgttcactcagttccagccacattgccctgggagctgctcccaccatggggcttcctaatgcactggtctctctcatgcagtggggcctctccctccttttactcagtgtctcccagcacccacctcctccagagccttccctgaccaccacacctacacctaggcccttcctcctccacgctccctcctccaccccggcctcctacccacgtgtcacttctttatactcgctgccacctgaaattagatcatttatttacccctttatttgttcagtttgccttgtccgttagaatataagcttccaaagggcaggagctttgcctatattgttaggccgggcatacaatgagcactcaaaaaaatatttgatgagtgtatgaaagaacagactgggttatgtaattgtgcctacttacctatatgaccgtgtggtggggtttatggtgggtgtggtggtgatggctatagggctataagcaaatttgggacagggagtctaagaaatgttcttaaattttagtaagcaaagcatcctctacagaacctgtcttaaaacatgaaagttccttagtgctacccccagaggtatgatttggtaggtcaaggatagggcctggaaattcacattcttgttaagatgttcttcatccggggtttgttgaccaccttttcagaagatttttgctctgtagctgtactacccaatgcagtagttcgtagtcagtgtggctcctgagcccttgaagtgtagctcctctgaactgagacgtgctgtaaatgtaaattgcacaccggagtttgaagagttaatacaaagaaaaaggaatgcaaaacatctcattaataatgctttacactgattacatattgaaatggtaatcttgtagatatagtgcgttaaataaaatatactgttaggcttaatttcacgtctttatacttttaatgtggctactagaaaaatttaaataacatattcagctcacattatactcctattgaacagagctgatctataagttccatggaagatggcaagtcttcgcagctgaaataaaggctggatcccattctacgggctcatctttagcaatgatttcttgcagacgatattgaaaaatgtggcaatgaaagttaccacaagcatcaaaccagtcctgcctaaatctggaaaatagttatctgaggctgttagcatatgatcatgagagcgtttcaccatggatttctgatcacagatgtggcacattattaaaatatcacttttacagtcaccctagaggctagggttatctgaatatggagaaagaaacagcttgtggagctgttgtataaatgaaattactagaaagtaatgcactcaattgcatattggctcggggggttattcttattaaaatgtttagagaggactttctgttcatttctgcagaattgctcttcaaattaagaatttgcttgacacgctaatagaccacagtcccaagagaagtttatccttttttcttcttatccttgctaagcacttagatgctctgctgataggtagcatatattgtctatatgaagcttttgtgttaacattgactagtcctgcaagttggcacactcttacttggcctaaaagaaatcagcaccaggctttaagaaaatcagatgatctacctaaaggaacacaactctgtctctcttttgacaattgttgtaaacaaattttaatggaaatttgccttaattgtgaagaagttgctgctaaaatggacttgccattaatggactggaacccattgcataagcagaatgaaatataagccttctcaggattcacacttataaaaaaccattcagccaatcaacaagagggcaaaagaacaaacatttgatgtgtaattacttaatttagtgcatatgcatttgggtcctcaatgtcagcactatggcaaccagaacatggccacaataactgtctggaaatgtctattcttacctggacccagcaggccatgccccactgattatataatctccctctctccttgttacggtctgaatgcttgcatccctcaaaaattcatgtgttgaaatcctaacccccaaggtgatgatattaggaggtcggccttttgagaggtaattaggtcatgaagacagcatcctcatgaatgggattagtgtccttataaaataggcccaagggagctcattcactttgtccaccatgtgagaacacagcgagagggcaccatttatgcaccaggaaatgggccttttccagacaatctgtcggtgcctggatcttggacttcacagcctctagaactgtgagaaattaatttgttttttataagccaccaaatctatggttttttttatagaaaccgtaatggactaaaacactccctaattatatttaaacttatcagtgcactgggcagtgacatattaaaagaatgctggccaacgtaattgacaccataaggctggatgattcttgtaattttcagcctcagaaaaaggctggggagaggagtcaggggaaaggaggtggtgtgtgtgtgtgtgtgtgtgtgtgtgtgtgtgtgtgtggtacggtggatgcctgctgagagagaaagagctataataacattctgtggttcagctgacacatcctttctgcatcccctccaatcacctgggttaatggggacctcgctaatgtctgaacctcatctcattttaaccttttgtttcaaagcctctcttttcatgacttccccgccttcatttttcccatatggtggggttattattaagacattaaatgagagtggacaggtaggcaaaggaggtgggttgcaggggagttgagggttgcctgtgtacttttctagactgttccacttcacatcagtgaaatattcccaattgatactatcatgaaacaaagcaaatgaaatgctgagcacggagcttcgtcttgatgaaatgctgaaagaaaagaaaggaaaaataaagtagccattatttttgcccttcctcccacccccatgtttactactcttatttctcttttgtattgttgtgttggaagcacagcatcagaaaaactcccagttttgagagataactcagtgtttagttcacttaaacctgagaaaggagaagaggatgccaccgtgaggtccaggacgtaaagaggaaaaaaacagacaaaaaaatccatatgaaatgaaaatgtgaaagaggcgctttcgagcagatgagtgttgtagattacagtgttgagagctgtttgtgtccagagctgcttgctgcacctggcgggataaacactggtctaacagaggatccttgtttcaaggaggctgccttttatttggggggacaaaattgttcttgaaagctgctcagtggttcaagctacagcatggtggactagcagaatggactccagggcctccgaggagacagtgactgctgccagaaatagtcaaggatagaaaggaaggacttcactgaggcctgggagaagattatggaatgggactgacagcagtgacggggagtaaaagggggtgtctgggggaattgtgccccatggtgagagctagagggttcacaaagacttaacccgacgcatctctctcaccctggagattgggcccgttcaatctaactggatggctataatttaaaaggtttaggtattatgacaaacatggatatattaggtgatagcaatgcaaaatgcatatggcttcttgatataaaacacaagacttgaaagcagcatctttggctgggtactacagccaccctcctctgtcactaagggaggctttggtggaaagggctgagagcctctagactgtgaacaaaagtaggcacagaagaacagttggagataataagtaaaccatcttgacaggaatgaagaatttcctgaaaggaaggtccctgagttaggttgttggatgctttcagtagtgagttattgaaagtgtttggggggtgtgtgtgtgtgtgtgtatgtgcagtatgtgtgtgt

SEQ ID NO: 2 - protein, 161 aa, homo sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 3 - DNA, 873 bp, Homo sapiens
acttccccttcctgtacagggcaggttgtgcagctggaggcagagcagtcctctctggggagcctgaagcaaacatggatcaagaaactgtaggcaatgttgtcctgttggccatcgtcaccctcatcagcgtggtccagaatggattctttgcccataaagtggagcacgaaagcaggacccagaatgggaggagcttccagaggaccggaacacttgcctttgagcgggtctacactgccaaccagaactgtgtagatgcgtaccccactttcctcgctgtgctctggtctgcggggctactttgcagccaagttcctgctgcgtttgctggactgatgtacttgtttgtgaggcaaaagtactttgtcggttacctaggagagagaacgcagagcacccctggctacatatttgggaaacgcatcatactcttcctgttcctcatgtccgttgctggcatattcaactattacctcatcttctttttcggaagtgactttgaaaactacataaagacgatctccaccaccatctcccctctacttctcattccctaactctctgctgaatatggggttggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttcttcacttatcctgttctctgaagatgttttgtgaccaggtttgtgttttcttaaaataaaatgcagagacatgtttt

SEQ ID NO: 4 - DNA, 20 bp, Homo sapiens
acggtgatgacgcctacatt

SEQ ID NO: 5 - DNA, 23 bp, Homo sapiens
tcacatggaccaattacctagaa

SEQ ID NO: 6 - DNA, 21 bp, Homo sapiens
caaatttcagatgtgccaacc

SEQ ID NO: 7 - DNA, 20 bp, Homo sapiens
acggtgatgacgcctacatt

SEQ ID NO: 8 - DNA, 18 bp, Homo sapiens
accagcctttgcttagga

SEQ ID NO: 9 - DNA, 24 bp, Homo sapiens
acattctagtgctacagggtactc

SEQ ID NO: 10 - DNA, 21 bp, Homo sapiens
tgttctgcacacgaacattct

SEQ ID NO: 11 - DNA, 20 bp, Homo sapiens
tcctgagtcctctccacctg

SEQ ID NO: 12 - DNA, 24 bp, Homo sapiens
tgggaattaatgaagaacaacaaa

SEQ ID NO: 13 - DNA, 23 bp, Homo sapiens
catgtttcgaagaactcaagagg

SEQ ID NO: 14 - DNA, 25 bp, Homo sapiens
aaattacttcatcttgacgataaca

SEQ ID NO: 15 - DNA, 20 bp, Homo sapiens
ctattggggactgcagagag

SEQ ID NO: 16 - DNA, 20 bp, Homo sapiens
gggactgcagagagcagaag

SEQ ID NO: 17 - DNA, 21 bp, Homo sapiens
caagaagggaaattcctacgc

SEQ ID NO: 18 - DNA, 20 bp, Homo sapiens
agccagtgtccacaaggaag

SEQ ID NO: 19 - DNA, 21 bp, Homo sapiens
gagggtgagacacatctctgg

SEQ ID NO: 20 - DNA, 20 bp, Homo sapiens
aatcgtgcctcagttccatc

SEQ ID NO: 21 - DNA, 20 bp, Homo sapiens
ccaccaggaacaacacacac

SEQ ID NO: 22 - DNA, 18 bp, Homo sapiens
ttgctctccagcctgggc

SEQ ID NO: 23 - DNA, 18 bp, Homo sapiens
ttcctctggctgcctgcg

SEQ ID NO: 24 - DNA, 20 bp, Homo sapiens
tttgattccgtggtccatta

SEQ ID NO: 25 - DNA, 21 bp, Homo sapiens
ttatttggtcggtgcaccttt

SEQ ID NO: 26 - DNA, 22 bp, Homo sapiens
ggtaggttgaaatgggctaaca

SEQ ID NO: 27 - DNA, 21 bp, Homo sapiens
tcatgacaaggtgttggattt

SEQ ID NO: 28 - DNA, 20 bp, Homo sapiens
cctcctctgccatgaagcta

SEQ ID NO: 29 - DNA, 20 bp, Homo sapiens
ctatttggtctgcgggttgt

SEQ ID NO: 30 - DNA, 21 bp, Homo sapiens
tttgagcccagatctaagcaa

SEQ ID NO: 31 - DNA, 22 bp, Homo sapiens
aaatgttaatgtcaccgacaaa

SEQ ID NO: 32 - DNA, 20 bp, Homo sapiens
tactgggttatcgcctgacc

SEQ ID NO: 33 - DNA, 20 bp, Homo sapiens
ccaatggacctcttggacat

SEQ ID NO: 34 - DNA, 25 bp, Homo sapiens
tttgaatgttcatatatttgtggtg

SEQ ID NO: 35 - DNA, 23 bp, Homo sapiens
ccctcgtaatgaaacctatttga

SEQ ID NO: 36 - DNA, 20 bp, Homo sapiens
tttcggcacagtcctcaata

SEQ ID NO: 37 - DNA, 16 bp, Homo sapiens
cagggtgtggtgacat

SEQ ID NO: 38 - DNA, 26 bp, Homo sapiens
tgtttctttctttctctctctctttc

SEQ ID NO: 39 - DNA, 24 bp, Homo sapiens
aaatgagttcaatgagttgtggtt

SEQ ID NO: 40 - DNA, 20 bp, Homo sapiens
cagagaggaacaggcagagg

SEQ ID NO: 41 - DNA, 20 bp, Homo sapiens
agtggctgggaagccttatt

SEQ ID NO: 42 - DNA, 23 bp, Homo sapiens
aggtgagagaacaaacctgtctt

SEQ ID NO: 43 - DNA, 20 bp, Homo sapiens
gccttccttctaaggccaac

SEQ ID NO: 44 - DNA, 25 bp, Homo sapiens
tgttatacatttcaatttcacctca

SEQ ID NO: 45 - DNA, 18 bp, Homo sapiens
gtactccagccgggcaac

SEQ ID NO: 46 - DNA, 27 bp, Homo sapiens
ttgttcagtgctctatagttacaaagt

SEQ ID NO: 47 - DNA, 21 bp, Homo sapiens
ggtcacaaagctatgcgatta

SEQ ID NO: 48 - DNA, 25 bp, Homo sapiens
tcaacaagtggattaagaaactgtg

SEQ ID NO: 49 - DNA, 23 bp, Homo sapiens
ctgtttatggctgagaagtatgc

SEQ ID NO: 50 - DNA, 17 bp, Homo sapiens
tagcagggtgcagtcta

SEQ ID NO: 51 - DNA, 20 bp, Homo sapiens
accataccaccaccaccatc

SEQ ID NO: 52 - DNA, 19 bp, Homo sapiens
actgtacttctgcctgggc

SEQ ID NO: 53 - DNA, 21 bp, Homo sapiens
ttttgtaatgcctcaaccatg

SEQ ID NO: 54 - DNA, 26 bp, Homo sapiens
ctgtagactttatccctgacttactg

SEQ ID NO: 55 - DNA, 24 bp, Homo sapiens
caatgaatgatgaagattccactc

SEQ ID NO: 56 - DNA, 23 bp, Homo sapiens
tgacaccatgtcttactgtttgc

SEQ ID NO: 57 - DNA, 25 bp, Homo sapiens
gaggatacaatgagaaccaaatctc

SEQ ID NO: 58 - DNA, 20 bp, Homo sapiens
ccacagaatgctccaaaggt

SEQ ID NO: 59 - DNA, 22 bp, Homo sapiens
gagttcaagtgatggatgacga

SEQ ID NO: 60 - DNA, 24 bp, Homo sapiens
cagatagatgaataggtggatgga

SEQ ID NO: 61 - DNA, 20 bp, Homo sapiens
cactgttccaagtgctttgc

SEQ ID NO: 62 - DNA, 19 bp, Homo sapiens
gcagggcaaactgccttat

SEQ ID NO: 63 - DNA, 23 bp, Homo sapiens
tttggtgaaatgtctgtttatgg

SEQ ID NO: 64 - DNA, 18 bp, Homo sapiens
ctcaacctggcttctact

SEQ ID NO: 65 - DNA, 20 bp, Homo sapiens
tactccttaataaactcccc

SEQ ID NO: 66 - DNA, 17 bp, Homo sapiens
tatgcgttgtgtgtgtg

SEQ ID NO: 67 - DNA, 22 bp, Homo sapiens
gggccttagattcttgtagtgg

SEQ ID NO: 68 - DNA, 20 bp, Homo sapiens
ctcgcatctcgcttctcact

SEQ ID NO: 69 - DNA, 20 bp, Homo sapiens
ctcaagggtccagtggtttg

SEQ ID NO: 70 - DNA, 20 bp, Homo sapiens
tgtccagactgcctcctaca

SEQ ID NO: 71 - DNA, 20 bp, Homo sapiens
tgcaacacctggttcacaat

SEQ ID NO: 72 - DNA, 24 bp, Homo sapiens
cacagtgagactctatctcaaaaa

SEQ ID NO: 73 - DNA, 21 bp, Homo sapiens
tcagactggcttagactgtgg

SEQ ID NO: 74 - DNA, 19 bp, Homo sapiens
aaattccaaaggccaggtg

SEQ ID NO: 75 - DNA, 23 bp, Homo sapiens
ccatacagtttcctaggttctgg

SEQ ID NO: 76 - DNA, 20 bp, Homo sapiens
cacctggccaaatgtttgtt

SEQ ID NO: 77 - DNA, 20 bp, Homo sapiens
tgcttgaatccagagactgc

SEQ ID NO: 78 - DNA, 20 bp, Homo sapiens
tttgcgagtccttgtggagt

SEQ ID NO: 79 - DNA, 20 bp, Homo sapiens
acagtccgctccctcctaat

SEQ ID NO: 80 - DNA, 18 bp, Homo sapiens
atgcttggccctcagttt

SEQ ID NO: 81 - DNA, 21 bp, Homo sapiens
ttggcaacccaagctaatatg

SEQ ID NO: 82 - DNA, 19 bp, Homo sapiens
ctccacagtgacagtgagg

SEQ ID NO: 83 - DNA, 17 bp, Homo sapiens
gagaggttcccaatccc

SEQ ID NO: 84 - DNA, 18 bp, Homo sapiens
catcaacctccccaccac

SEQ ID NO: 85 - DNA, 24 bp, Homo sapiens
tattttttcagtcccacagttagc

SEQ ID NO: 86 - DNA, 20 bp, Homo sapiens
cagctcctggccatatttct

SEQ ID NO: 87 - DNA, 20 bp, Homo sapiens
gagccatttctctgggtctg

SEQ ID NO: 88 - DNA, 20 bp, Homo sapiens
ggtccgtgtcaacccttaga

SEQ ID NO: 89 - DNA, 19 bp, Homo sapiens
caggttgatgggagggaaa

SEQ ID NO: 90 - DNA, 20 bp, Homo sapiens
cgggaaatgacagtgagacc

SEQ ID NO: 91 - DNA, 20 bp, Homo sapiens
tgcctagattctcccgtaag

SEQ ID NO: 92 - DNA, 16 bp, Homo sapiens
gtgcccagccagattc

SEQ ID NO: 93 - DNA, 16 bp, Homo sapiens
gcccccagtcaggttt

SEQ ID NO: 94 - DNA, 21 bp, Homo sapiens
tttctctctccacggaatgaa

SEQ ID NO: 95 - DNA, 21 bp, Homo sapiens
aacccattctcacagggtgta

SEQ ID NO: 96 - DNA, 20 bp, Homo sapiens
aggagtgtggcagctttgag

SEQ ID NO: 97 - DNA, 20 bp, Homo sapiens
tggattcccgtgagtaccag

SEQ ID NO: 98 - DNA, 17 bp, Homo sapiens
atgctgggatcacaggc

SEQ ID NO: 99 - DNA, 19 bp, Homo sapiens
aacctggtggacttttgct

SEQ ID NO: 100 - DNA, 20 bp, Homo sapiens
agcatttccaatggtgcttt

SEQ ID NO: 101 - DNA, 21 bp, Homo sapiens
catgttgatatgcctgaagga

SEQ ID NO: 102 - DNA, 20 bp, Homo sapiens
cactgtctgctgccactcat

SEQ ID NO: 103 - DNA, 27 bp, Homo sapiens
agagattatgtgatgtaccctctctat

SEQ ID NO: 104 - DNA, 20 bp, Homo sapiens
caagcctgggacacagaaat

SEQ ID NO: 105 - DNA, 20 bp, Homo sapiens
tttgcagacaccacaacaca

SEQ ID NO: 106 - DNA, 22 bp, Homo sapiens
atgacctagaaatgatactggc

SEQ ID NO: 107 - DNA, 20 bp, Homo sapiens
cagacaccacaacacacatt

SEQ ID NO: 108 - DNA, 20 bp, Homo sapiens
tggtttaaaaacctcatgcc

SEQ ID NO: 109 - DNA, 25 bp, Homo sapiens
atcccaaactctgtacttatgtagg

SEQ ID NO: 110 - DNA, 23 bp, Homo sapiens
tttgcacatacacataagcgaac

SEQ ID NO: 111 - DNA, 20 bp, Homo sapiens
cacaaatcccgtgcactaaa

SEQ ID NO: 112 - DNA, 20 bp, Homo sapiens
attcctgggctcatggtaca

SEQ ID NO: 113 - DNA, 20 bp, Homo sapiens
tgccgtcatctgctttagaa

SEQ ID NO: 114 - DNA, 20 bp, Homo sapiens
ccttggctgttgtgactggt

SEQ ID NO: 115 - DNA, 20 bp, Homo sapiens
cactcaggtgggaggatcac

SEQ ID NO: 116 - DNA, 21 bp, Homo sapiens
gctgtttccttggcttcttct

SEQ ID NO: 117 - DNA, 22 bp, Homo sapiens
cccatacttgagatgaccatga

SEQ ID NO: 118 - DNA, 20 bp, Homo sapiens
cactttgccagtagccttga

SEQ ID NO: 119 - DNA, 21 bp, Homo sapiens
ttgggaaagttaacccagaga

SEQ ID NO: 120 - DNA, 20 bp, Homo sapiens
tttgggaagagccatgagac

SEQ ID NO: 121 - DNA, 20 bp, Homo sapiens
ctctgggcattggaggatta

SEQ ID NO: 122 - DNA, 20 bp, Homo sapiens
tttgggaagagccatgagac

SEQ ID NO: 123 - DNA, 20 bp, Homo sapiens
aatgcccatgtgcactgtag

SEQ ID NO: 124 - DNA, 20 bp, Homo sapiens
gggagacaagtcaggtgagg

SEQ ID NO: 125 - DNA, 26 bp, Homo sapiens
ctgagtatggagtcttcatcattatc

SEQ ID NO: 126 - DNA, 23 bp, Homo sapiens
tcgtctcgaagaaagaaagaaga

SEQ ID NO: 127 - DNA, 20 bp, Homo sapiens
caccatgggttaattgcaca

SEQ ID NO: 128 - DNA, 20 bp, Homo sapiens
tgacgtgggttcaggttgta

SEQ ID NO: 129 - DNA, 20 bp, Homo sapiens
agtgcattggtgccttctct

SEQ ID NO: 130 - DNA, 20 bp, Homo sapiens
ggactgccaattctacagca

SEQ ID NO: 131 - DNA, 20 bp, Homo sapiens
tttccatgggaaatttggtc

SEQ ID NO: 132 - DNA, 22 bp, Homo sapiens
tgctactagatttgaccaacca

SEQ ID NO: 133 - DNA, 26 bp, Homo sapiens
gacttgtaaaggatttagtgatttcg

SEQ ID NO: 134 - DNA, 17 bp, Homo sapiens
gtggaaggcctctcttg

SEQ ID NO: 135 - DNA, 20 bp, Homo sapiens
tgcttcttgagggaaagcat

SEQ ID NO: 136 - DNA, 21 bp, Homo sapiens
cacgtggttcacctctctagg

SEQ ID NO: 137 - DNA, 17 bp, Homo sapiens
ttggccacttatttgtg

SEQ ID NO: 138 - DNA, 17 bp, Homo sapiens
cgatgagtgacagggct

SEQ ID NO: 139 - DNA, 17 bp, Homo sapiens
cctcgtgggtggaataa

SEQ ID NO: 140 - DNA, 20 bp, Homo sapiens
ttggccattagcaattagca

SEQ ID NO: 141 - DNA, 20 bp, Homo sapiens
cgtgggtggaataaatcagg

SEQ ID NO: 142 - DNA, 20 bp, Homo sapiens
gttgaggcaagagaatcact

SEQ ID NO: 143 - DNA, 19 bp, Homo sapiens
gcacatttacaccagggtg

SEQ ID NO: 144 - DNA, 21 bp, Homo sapiens
ccttcagaggatttccctttc

SEQ ID NO: 145 - DNA, 20 bp, Homo sapiens
ctggtttgactccagcttca

SEQ ID NO: 146 - DNA, 22 bp, Homo sapiens
tgttcaaacctaaggtgcttca

SEQ ID NO: 147 - DNA, 24 bp, Homo sapiens
gaaacaacaacaacaacaacaaca

SEQ ID NO: 148 - DNA, 20 bp, Homo sapiens
cctggcacggaatagacact

SEQ ID NO: 149 - DNA, 19 bp, Homo sapiens
ggcctcctttgctctgaag

SEQ ID NO: 150 - DNA, 21 bp, Homo sapiens
catccctgtggctgattaaga

SEQ ID NO: 151 - DNA, 20 bp, Homo sapiens
aacagttccagcccgttcta

SEQ ID NO: 152 - DNA, 22 bp, Homo sapiens
tttcaaaggaatatccaagtgc

SEQ ID NO: 153 - DNA, 24 bp, Homo sapiens
tggcgtaccatataaacagttctc

SEQ ID NO: 154 - DNA, 22 bp, Homo sapiens
tttcaaaggaatatccaagtgc

SEQ ID NO: 155 - DNA, 20 bp, Homo sapiens
aaacgtgacacttccacaca

SEQ ID NO: 156 - DNA, 20 bp, Homo sapiens
ttcaatgaaggtgccgaagt

SEQ ID NO: 157 - DNA, 17 bp, Homo sapiens
tgtctatcccaaagcaa

SEQ ID NO: 158 - DNA, 24 bp, Homo sapiens
gcaagactctgttgaagaagaaga

SEQ ID NO: 159 - DNA, 21 bp, Homo sapiens
tccctctgtttgagtttctcg

SEQ ID NO: 160 - DNA, 19 bp, Homo sapiens
aggcacagtcgctcatgtc

SEQ ID NO: 161 - DNA, 24 bp, Homo sapiens
aaactttagctaatggtggtcaaa

SEQ ID NO: 162 - DNA, 21 bp, Homo sapiens
tgtgattccagggagctatca

SEQ ID NO: 163 - DNA, 20 bp, Homo sapiens
taggtgtgtggaggacagca

SEQ ID NO: 164 - DNA, 23 bp, Homo sapiens
ccagtttcagttagccaagtctg

SEQ ID NO: 165 - DNA, 20 bp, Homo sapiens
gagagggaatgaatgcagga

SEQ ID NO: 166 - DNA, 25 bp, Homo sapiens
gagcatgtgtgactttcatattcag

SEQ ID NO: 167 - DNA, 22 bp, Homo sapiens
agtggctattcattgctacagg

SEQ ID NO: 168 - DNA, 20 bp, Homo sapiens
ttgctggatgctggtttcta

SEQ ID NO: 169 - DNA, 27 bp, Homo sapiens
aaagagagagagaaagagaaagaaaga

SEQ ID NO: 170 - DNA, 16 bp, Homo sapiens
ctggttgagcggcatt

SEQ ID NO: 171 - DNA, 16 bp, Homo sapiens
tgcagcctggatgaca

SEQ ID NO: 172 - DNA, 22 bp, Homo sapiens
cctatggaagcatagggaagaa

SEQ ID NO: 173 - DNA, 21 bp, Homo sapiens
cccacttctgagtctcctgat

SEQ ID NO: 174 - DNA, 20 bp, Homo sapiens
gggatgcagaaaggatgtgt

SEQ ID NO: 175-176 - skipped (2 entries)

SEQ ID NO: 177 - DNA, 20 bp, Homo sapiens
aagaatgctggccaacgtaa

SEQ ID NO: 178 - DNA, 17 bp, Homo sapiens
ctctcagcaggcatcca

SEQ ID NO: 179 - DNA, 19 bp, Homo sapiens
gccaacgtaattgacacca

SEQ ID NO: 180 - DNA, 18 bp, Homo sapiens
ccttaggccccataatct

SEQ ID NO: 181 - DNA, 21 bp, Homo sapiens
caaattcctcaattgcaaaat

SEQ ID NO: 182 - DNA, 20 bp, Homo sapiens
ggtcattcagggagccattc

SEQ ID NO: 183 - DNA, 25 bp, Homo sapiens
ccattatatttcaccaagaggctgc

SEQ ID NO: 184 - DNA, 20 bp, Homo sapiens
tgcctggtcatctacccatt

SEQ ID NO: 185 - DNA, 20 bp, Homo sapiens
tctactgcagcgctgatctt

SEQ ID NO: 186 - DNA, 22 bp, Homo sapiens
catttatgaatggaggtgaagc

SEQ ID NO: 187 - DNA, 20 bp, Homo sapiens
atgggagctcaaagggaaat

SEQ ID NO: 188 - DNA, 20 bp, Homo sapiens
cagcaggaagatggacaggt

SEQ ID NO: 189 - DNA, 21 bp, Homo sapiens
cacactgcatcacacataccc

SEQ ID NO: 190 - DNA, 18 bp, Homo sapiens
tatgccagtatgcctgct

SEQ ID NO: 191 - DNA, 19 bp, Homo sapiens
gtcacatcagtccatttgc

SEQ ID NO: 192 - DNA, 21 bp, Homo sapiens
ccaaagcaagtaacctcctca

SEQ ID NO: 193 - DNA, 20 bp, Homo sapiens
aaacaatcactgccctctgg

SEQ ID NO: 194 - DNA, 22 bp, Homo sapiens
tgatgaaattgcctagtgatgc

SEQ ID NO: 195 - DNA, 20 bp, Homo sapiens
ggatccaatcgtacgctacc

SEQ ID NO: 196 - DNA, 20 bp, Homo sapiens
cgaatgggtgactaacagca

SEQ ID NO: 197 - DNA, 19 bp, Homo sapiens
ctggagtgcagggacatga

SEQ ID NO: 198 - DNA, 26 bp, Homo sapiens
aaagaaatattccaagaagaaagaaa

SEQ ID NO: 199 - DNA, 23 bp, Homo sapiens
ttgcacaactttgtgtagagcat

SEQ ID NO: 200 - DNA, 25 bp, Homo sapiens
gggtatgtctttattctcggcagta

SEQ ID NO: 201 - DNA, 22 bp, Homo sapiens
gtgcattcacagaccagtcatt

SEQ ID NO: 202 - DNA, 21 bp, Homo sapiens
gggcttgaaggcactaaatgt

SEQ ID NO: 203 - DNA, 22 bp, Homo sapiens
ccaagcagtaattccttcctca

SEQ ID NO: 204 - DNA, 20 bp, Homo sapiens
acctaaacaccacggactgg

SEQ ID NO: 205 - DNA, 22 bp, Homo sapiens
caggtatcgacattcttccaaa

SEQ ID NO: 206 - DNA, 22 bp, Homo sapiens
tgggaagccagtaaagtaggaa

SEQ ID NO: 207 - DNA, 23 bp, Homo sapiens
aaagagactccacacatccattt

SEQ ID NO: 208 - DNA, 21 bp, Homo sapiens
agggctattcctcaaggtgtt

SEQ ID NO: 209 - DNA, 21 bp, Homo sapiens
tgctaacactaccctcgcaat

SEQ ID NO: 210 - DNA, 20 bp, Homo sapiens
gggcaggaatctctgaagtg

SEQ ID NO: 211 - DNA, 20 bp, Homo sapiens
ctccactgagaagccaagga

SEQ ID NO: 212 - DNA, 19 bp, Homo sapiens
aggccaagctggtccatag

SEQ ID NO: 213 - DNA, 20 bp, Homo sapiens
tctctcaaagcctcgctctc

SEQ ID NO: 214 - DNA, 20 bp, Homo sapiens
cctttgaggctggatctgtt

SEQ ID NO: 215 - DNA, 23 bp, Homo sapiens
tttccttatcattcattccctca

SEQ ID NO: 216 - DNA, 22 bp, Homo sapiens
agatattgtctccgttccatga

SEQ ID NO: 217 - DNA, 22 bp, Homo sapiens
cccagatataaggacctggcta

SEQ ID NO: 218 - DNA, 23 bp, Homo sapiens
tttaagccctgtggaatgtattt

SEQ ID NO: 219 - DNA, 21 bp, Homo sapiens
gacattgcaggtcaagtaggg

SEQ ID NO: 220 - DNA, 20 bp, Homo sapiens
tgcataaggctggagacaga

SEQ ID NO: 221 - DNA, 19 bp, Homo sapiens
cacagcagatgggagcaaa

SEQ ID NO: 222 - DNA, 20 bp, Homo sapiens
gtgcatgtgcataccagacc

SEQ ID NO: 223 - DNA, 20 bp, Homo sapiens
ggcaagatgacctctggaaa

SEQ ID NO: 224 - DNA, 20 bp, Homo sapiens
gtccactgcagcacacagag

SEQ ID NO: 225 - DNA, 23 bp, Homo sapiens
gcactggtagatacatgctaacg

SEQ ID NO: 226 - DNA, 19 bp, Homo sapiens
gggtatcttggccaggtgt

SEQ ID NO: 227 - DNA, 20 bp, Homo sapiens
tggctaagcacaatcccttt

SEQ ID NO: 228 - DNA, 22 bp, Homo sapiens
tttgtgttccaggtgagaattg

SEQ ID NO: 229 - DNA, 20 bp, Homo sapiens
gaaccatatcccaaggcact

SEQ ID NO: 230 - DNA, 21 bp, Homo sapiens
aacccaaatcaacaaaccaga

SEQ ID NO: 231 - DNA, 21 bp, Homo sapiens
aatgaattctgggtcacatgc

SEQ ID NO: 232 - DNA, 22 bp, Homo sapiens
ttgttcccacattcattctaca

SEQ ID NO: 233 - DNA, 20 bp, Homo sapiens
ttaaactcgtggcaaagacg

SEQ ID NO: 234 - DNA, 18 bp, Homo sapiens
caccatgcctggctcttt

SEQ ID NO: 235 - DNA, 22 bp, Homo sapiens
aacttctccagttgtgtggttg

SEQ ID NO: 236 - DNA, 18 bp, Homo sapiens
agctgagctcatgccact

SEQ ID NO: 237 - DNA, 20 bp, Homo sapiens
caagaccttgtgcatttgga

SEQ ID NO: 238 - DNA, 20 bp, Homo sapiens
agccagacatggtagtgtgc

SEQ ID NO: 239 - DNA, 22 bp, Homo sapiens
gcaataactcacacatcagcaa

SEQ ID NO: 240 - DNA, 20 bp, Homo sapiens
cctaccattgacactctcag

SEQ ID NO: 241 - DNA, 16 bp, Homo sapiens
tagggccatccattct

SEQ ID NO: 242 - DNA, 20 bp, Homo sapiens
accaagatatgaaggccaaa

SEQ ID NO: 243 - DNA, 22 bp, Homo sapiens
cctccagctagaacaatgtgaa

SEQ ID NO: 244 - DNA, 21 bp, Homo sapiens
tgtccatagctgtagccctgt

SEQ ID NO: 245 - DNA, 20 bp, Homo sapiens
ctcaatgggcatctttaggc

SEQ ID NO: 246 - DNA, 22 bp, Homo sapiens
tgtaattcaacgactggtgtcc

SEQ ID NO: 247 - DNA, 20 bp, Homo sapiens
agcttctgatggttgctggt

SEQ ID NO: 248 - DNA, 22 bp, Homo sapiens
caaacaaacaaacaagcaaacc

SEQ ID NO: 249 - DNA, 21 bp, Homo sapiens
tggacgtttctttcagtgagg

SEQ ID NO: 250 - DNA, 23 bp, Homo sapiens
tgataacttaccagcatgtgagc

SEQ ID NO: 251 - DNA, 21 bp, Homo sapiens
tcacctcacctaaggatctgc

SEQ ID NO: 252 - DNA, 20 bp, Homo sapiens
catgcaattgcccaatagag

SEQ ID NO: 253 - DNA, 22 bp, Homo sapiens
ttgggcttgtctacctagttca

SEQ ID NO: 254 - DNA, 21 bp, Homo sapiens
tgggttcctcatactggagtg

SEQ ID NO: 255 - DNA, 20 bp, Homo sapiens
gcctgagctccaagctcttt

SEQ ID NO: 256 - DNA, 20 bp, Homo sapiens
gctgcacgtatttgttggtg

SEQ ID NO: 257 - DNA, 20 bp, Homo sapiens
aaacagcagaaatgggaacc

SEQ ID NO: 258 - DNA, 20 bp, Homo sapiens
ccgtgggctatcaatttctg

SEQ ID NO: 259 - DNA, 21 bp, Homo sapiens
aagatgcaatctggtttccaa

SEQ ID NO: 260 - DNA, 20 bp, Homo sapiens
cccaagactgaggaggtcaa

SEQ ID NO: 261 - DNA, 20 bp, Homo sapiens
gctgacggagaggaaagaga

SEQ ID NO: 262 - DNA, 20 bp, Homo sapiens
tgacaagggtgtggttatgg

SEQ ID NO: 263 - DNA, 20 bp, Homo sapiens
ccgcactttctcttctggac

SEQ ID NO: 264 - DNA, 20 bp, Homo sapiens
tgagaagcctgggcattaag

SEQ ID NO: 265 - DNA, 20 bp, Homo sapiens
acaagctcatccagggaaag

SEQ ID NO: 266 - DNA, 21 bp, Homo sapiens
ttggaaaggaagaaaggaagg

SEQ ID NO: 267 - DNA, 21 bp, Homo sapiens
ttgaaacctaaatgccacctg

SEQ ID NO: 268 - DNA, 20 bp, Homo sapiens
acctgttgtatggcagcagt

SEQ ID NO: 269 - DNA, 20 bp, Homo sapiens
ggttgactctttccccaact

SEQ ID NO: 270 - DNA, 19 bp, Homo sapiens
agagctgatctggccgaag

SEQ ID NO: 271 - DNA, 21 bp, Homo sapiens
ggtggacacagaatccacact

SEQ ID NO: 272 - DNA, 18 bp, Homo sapiens
ggcctgaaaggtatcctc

SEQ ID NO: 273 - DNA, 18 bp, Homo sapiens
tcccaccataagcacaag

SEQ ID NO: 274 - DNA, 22 bp, Homo sapiens
tcaacctaggattggcattaca

SEQ ID NO: 275 - DNA, 21 bp, Homo sapiens
tctaggatttgtgcctttcca

SEQ ID NO: 276 - DNA, 23 bp, Homo sapiens
gacgtcttaggattgacttctgc

SEQ ID NO: 277 - DNA, 25 bp, Homo sapiens
ccaaatacacattcttaaagggaaa

SEQ ID NO: 278 - DNA, 20 bp, Homo sapiens
gactgcagatcgtgggactt

SEQ ID NO: 279 - DNA, 21 bp, Homo sapiens
ttctccagagaaaccaaacca

SEQ ID NO: 280 - DNA, 20 bp, Homo sapiens
attcgtgcagctgtttctgc

SEQ ID NO: 281 - DNA, 22 bp, Homo sapiens
gcatgacattgtaaatggagga

SEQ ID NO: 282 - DNA, 20 bp, Homo sapiens
ggtgggaatgtgtgactgaa

SEQ ID NO: 283 - DNA, 22 bp, Homo sapiens
ccaggtacaacattctcctgat

SEQ ID NO: 284 - DNA, 16 bp, Homo sapiens
tgcaggtgggagtcaa

SEQ ID NO: 285 - DNA, 24 bp, Homo sapiens
aaataacaagaagtgaccttccta

SEQ ID NO: 286 - DNA, 20 bp, Homo sapiens
tgttctcctcaccctgctct

SEQ ID NO: 287 - DNA, 22 bp, Homo sapiens
tttcaggctaggaagatccttt

SEQ ID NO: 288 - DNA, 21 bp, Homo sapiens
aaaggatgcattcggttagag

SEQ ID NO: 289 - DNA, 20 bp, Homo sapiens
actgtcctgtgcctgtgctt

SEQ ID NO: 290 - DNA, 25 bp, Homo sapiens
cctgaataggtggaattaagatcaa

SEQ ID NO: 291 - DNA, 22 bp, Homo sapiens
tcaaggagcatacacacacaca

SEQ ID NO: 292 - DNA, 20 bp, Homo sapiens
gtccacctaatggctcattc

SEQ ID NO: 293 - DNA, 21 bp, Homo sapiens
caagaagcactcatgtttgtg

SEQ ID NO: 294 - DNA, 19 bp, Homo sapiens
agcctgtgattggctgaga

SEQ ID NO: 295 - DNA, 20 bp, Homo sapiens
ggcttacagctgcctccttt

SEQ ID NO: 296 - DNA, 21 bp, Homo sapiens
cccacagagcactttgttaga

SEQ ID NO: 297 - DNA, 21 bp, Homo sapiens
gcctcccttaagctgttatgc

SEQ ID NO: 298 - DNA, 23 bp, Homo sapiens
cactctttactgccaatcactcc

SEQ ID NO: 299 - DNA, 19 bp, Homo sapiens
gccgtgtgggtgtatgaat

SEQ ID NO: 300 - DNA, 22 bp, Homo sapiens
ttgtaccaggaaccaaagacaa

SEQ ID NO: 301 - DNA, 20 bp, Homo sapiens
cacagacagaggcacattga

SEQ ID NO: 302 - DNA, 20 bp, Homo sapiens
gctctggtcactcctgctgt

SEQ ID NO: 303 - DNA, 19 bp, Homo sapiens
catgcctggctgattgttt

SEQ ID NO: 304 - DNA, 16 bp, Homo sapiens
ccaacatcgggaactg

SEQ ID NO: 305 - DNA, 21 bp, Homo sapiens
tgcattctttaagtccatgtc

SEQ ID NO: 306 - DNA, 21 bp, Homo sapiens
cagcaactgacaactcatcca

SEQ ID NO: 307 - DNA, 20 bp, Homo sapiens
cctcaatcctcagctccaac

SEQ ID NO: 308 - DNA, 21 bp, Homo sapiens
tccttcacagcttcaaactca

SEQ ID NO: 309 - DNA, 22 bp, Homo sapiens
agtgagaagcttccatactggt

SEQ ID NO: 310 - DNA, 20 bp, Homo sapiens
gccaaccgttagacaaatga

SEQ ID NO: 311 - DNA, 21 bp, Homo sapiens
ctacatgtgcaccacaacacc

SEQ ID NO: 312 - DNA, 19 bp, Homo sapiens
agtttattgccgccgagag

SEQ ID NO: 313 - DNA, 19 bp, Homo sapiens
acccaccacattcacaagc

SEQ ID NO: 314 - DNA, 20 bp, Homo sapiens
cgattgccatgtctctttga

SEQ ID NO: 315 - DNA, 20 bp, Homo sapiens
gagatctggcctggatttgt

SEQ ID NO: 316 - DNA, 21 bp, Homo sapiens
tgaggccagccttacctctat

SEQ ID NO: 317 - DNA, 18 bp, Homo sapiens
ccagacatggtggcttgt

SEQ ID NO: 318 - DNA, 20 bp, Homo sapiens
gaaggaaggaagggaaggaa

SEQ ID NO: 319 - DNA, 21 bp, Homo sapiens
aaggatgagaagagtccatgc

SEQ ID NO: 320 - DNA, 23 bp, Homo sapiens
aaataccctttgaacagacacac

SEQ ID NO: 321 - DNA, 20 bp, Homo sapiens
tagctgagcatggtggtacg

SEQ ID NO: 322 - DNA, 22 bp, Homo sapiens
aaagacaagacagcaatccaaa

SEQ ID NO: 323 - DNA, 20 bp, Homo sapiens
gcagaacccaggctacagat

SEQ ID NO: 324 - DNA, 23 bp, Homo sapiens
tcattgtcagcacagaatgaact

SEQ ID NO: 325 - DNA, 20 bp, Homo sapiens
ggagggagggaagaaagaga

SEQ ID NO: 326 - DNA, 19 bp, Homo sapiens
gcaacacagtgaaagccca

SEQ ID NO: 327 - DNA, 19 bp, Homo sapiens
acaggagcatgccaccatg

SEQ ID NO: 328 - DNA, 22 bp, Homo sapiens
gggaagaggagattgacttgtt

SEQ ID NO: 329 - DNA, 20 bp, Homo sapiens
ggaacaccatcattccaacc

SEQ ID NO: 330 - DNA, 20 bp, Homo sapiens
tacaagctccaccgtccttc

SEQ ID NO: 331 - DNA, 20 bp, Homo sapiens
tgagttgctgcctcttcaaa

SEQ ID NO: 332 - DNA, 20 bp, Homo sapiens
tgctaatgggccaaggaata

SEQ ID NO: 333 - DNA, 23 bp, Homo sapiens
gctaaatgtcctcatgaatagcc

SEQ ID NO: 334 - DNA, 20 bp, Homo sapiens
tgtcctgcagacagatggtc

SEQ ID NO: 335 - DNA, 20 bp, Homo sapiens
cctccggagtagctggatta

SEQ ID NO: 336 - DNA, 20 bp, Homo sapiens
gagactggccctcattcttg

SEQ ID NO: 337 - DNA, 25 bp, Homo sapiens
aagaagccagagacaaagaaataca

SEQ ID NO: 338 - DNA, 24 bp, Homo sapiens
catctatctttggattcagtggtg

SEQ ID NO: 339 - DNA, 20 bp, Homo sapiens
tgctcccaacatcttaccag

SEQ ID NO: 340 - DNA, 23 bp, Homo sapiens
tgtcctctggtcatttctatggt

SEQ ID NO: 341 - DNA, 23 bp, Homo sapiens
catgaatgagaagtgatgaatgg

SEQ ID NO: 342 - DNA, 20 bp, Homo sapiens
aacacgggaaattccaacag

SEQ ID NO: 343 - DNA, 23 bp, Homo sapiens
tgaagaactgaaattgccagtaa

SEQ ID NO: 344 - DNA, 22 bp, Homo sapiens
cagacactgtaaactggcttcg

SEQ ID NO: 345 - DNA, 20 bp, Homo sapiens
gccacattgctatcagcgta

SEQ ID NO: 346 - DNA, 21 bp, Homo sapiens
tgtcataggcttgcggtattt

SEQ ID NO: 347 - DNA, 20 bp, Homo sapiens
ttggtagggtcctttccttt

SEQ ID NO: 348 - DNA, 20 bp, Homo sapiens
gcctgctcactgttgtttga

SEQ ID NO: 349 - DNA, 21 bp, Homo sapiens
cggttatcagagactggtggt

SEQ ID NO: 350 - DNA, 21 bp, Homo sapiens
ggcttatttcatgtacggcta

SEQ ID NO: 351 - DNA, 26 bp, Homo sapiens
ggttaaactctacttagtcctgatgc

SEQ ID NO: 352 - DNA, 20 bp, Homo sapiens
gaactctgcaggcacctctt

SEQ ID NO: 353 - DNA, 20 bp, Homo sapiens
cctgaagcgcttgtactgaa

SEQ ID NO: 354 - DNA, 20 bp, Homo sapiens
tgttgcgtactcagcccata

SEQ ID NO: 355 - DNA, 20 bp, Homo sapiens
gacaggtgtcaaacgggtct

SEQ ID NO: 356 - DNA, 20 bp, Homo sapiens
ttggcttctcgctctttctt

SEQ ID NO: 357 - DNA, 20 bp, Homo sapiens
agccatcagtcacatgcaaa

SEQ ID NO: 358 - DNA, 20 bp, Homo sapiens
agatctccagggcagaggac

SEQ ID NO: 359 - DNA, 20 bp, Homo sapiens
ccttcctccctccttctctc

SEQ ID NO: 360 - DNA, 21 bp, Homo sapiens
cgtcattgatcccaatcatct

SEQ ID NO: 361 - DNA, 20 bp, Homo sapiens
ggctgatagcctcccttgta

SEQ ID NO: 362 - DNA, 21 bp, Homo sapiens
gagagagagcagcttgcatgt

SEQ ID NO: 363 - DNA, 20 bp, Homo sapiens
ggctgatagcctcccttgta

SEQ ID NO: 364 - DNA, 20 bp, Homo sapiens
acctttcaagcttccggttt

SEQ ID NO: 365 - DNA, 20 bp, Homo sapiens
ttccatccgtccatctatcc

SEQ ID NO: 366 - DNA, 23 bp, Homo sapiens
ttaaagtcacttgtctgtggtca

SEQ ID NO: 367 - DNA, 27 bp, Homo sapiens
tttgtaggaatcaagtcaaataatgta

SEQ ID NO: 368 - DNA, 20 bp, Homo sapiens
caaacatcacactgggcaaa

SEQ ID NO: 369 - DNA, 21 bp, Homo sapiens
tgctttggaatctttcttgct

SEQ ID NO: 370 - DNA, 19 bp, Homo sapiens
ctgccaggatgtcagcatt

SEQ ID NO: 371 - DNA, 23 bp, Homo sapiens
tccacactttctcatcacctaaa

SEQ ID NO: 372 - DNA, 20 bp, Homo sapiens
ctttcggaagcttgagccta

SEQ ID NO: 373 - DNA, 20 bp, Homo sapiens
cccaagaccactgccatatt

SEQ ID NO: 374 - DNA, 22 bp, Homo sapiens
tgacaggtttgggtatattgga

SEQ ID NO: 375 - DNA, 20 bp, Homo sapiens
tgcttaatgtagtggcagca

SEQ ID NO: 376 - DNA, 20 bp, Homo sapiens
tcctgcctttgtgaattcct

SEQ ID NO: 377 - DNA, 20 bp, Homo sapiens
gttgaatgaggtgggcatta

SEQ ID NO: 378 - DNA, 21 bp, Homo sapiens
ccatttaatcctccagccatt

SEQ ID NO: 379 - DNA, 20 bp, Homo sapiens
gctccaccttgttaccctga

SEQ ID NO: 380 - DNA, 20 bp, Homo sapiens
acaaccctggaatctggact

SEQ ID NO: 381 - DNA, 22 bp, Homo sapiens
gaaggaaaggaaaggaaagaaa

SEQ ID NO: 382 - DNA, 23 bp, Homo sapiens
tgacaagactgaaacttcatcag

SEQ ID NO: 383 - DNA, 20 bp, Homo sapiens
gatgcttgctttgggaggta

SEQ ID NO: 384 - DNA, 19 bp, Homo sapiens
ttgaggacctgtcgttacg

SEQ ID NO: 385 - DNA, 21 bp, Homo sapiens
ttatagagcagttaaggcaca

SEQ ID NO: 386 - DNA, 21 bp, Homo sapiens
tgagggtggtaagcccttatt

SEQ ID NO: 387 - DNA, 21 bp, Homo sapiens
ggagttgtggcctctctctct

SEQ ID NO: 388 - DNA, 20 bp, Homo sapiens
aagcaaatatgcaaaattgc

SEQ ID NO: 389 - DNA, 23 bp, Homo sapiens
tccttctgtttcttgacttaaca

SEQ ID NO: 390 - DNA, 20 bp, Homo sapiens
tgctaagagggcagatctca

SEQ ID NO: 391 - DNA, 20 bp, Homo sapiens
ggctcatagccaatttctcc

SEQ ID NO: 392 - DNA, 21 bp, Homo sapiens
cggcattctcaataacctcaa

SEQ ID NO: 393 - DNA, 24 bp, Homo sapiens
tctttgatgaggatcaattagtgg

SEQ ID NO: 394 - DNA, 20 bp, Homo sapiens
acgcacacacacacacacac

SEQ ID NO: 395 - DNA, 22 bp, Homo sapiens
tgcctctgtaatcctgtgtagc

SEQ ID NO: 396 - DNA, 21 bp, Homo sapiens
gctctaaggtgggtcccaata

SEQ ID NO: 397 - DNA, 23 bp, Homo sapiens
gggaatgacaagatcagtttacc

SEQ ID NO: 398 - DNA, 396 bp, Homo sapiens
gattatatcccacctaccactgcagctccaggatccagcttcacaaacatttgttgaatgaatgaataagaaaagaggacacccccaaagaggctgcaagggaaaaagctacaaagacagaagcaccaggaaaaagtagggtcatgtaagtcaaagcaggaaaaaagttccatggtggggtggtcagcagtgtctaatrccacgaaggcacaaagtaggataaaggttaaaaatcagcctttggttttggcaaatatgaagcttatcggtagccttagcgagaacaattccatcagggagcagaagctaactgcagtgggttgagtcatcaagcaggcataaggaagtagggataccccattataagctactctttcaagaagctcaaatctgaag

SEQ ID NO: 399 - DNA, 396 bp, Homo sapiens
acaaaaattaccatcatatgctgtcatgcatgtctgccagtctatttatcatattatttaagaaacaaacatttattgaagatttatcatgtgctcagcactgccaaagaggaaataaagagcataatatctattcttagaaaataacattaacacaaatagaaaacaagaaaccataatgttaaaaatattacatagyaacacagaaagacaatgtataattatacatacgcactaaagcaaagataacataatttataaattatgaggtacagaatagttagattctgaaaattaaaataatcaggaaaaacttcatgaagatgagatctgggctggatcccaaaggataggcaggtggatcatgtagaacaggggaaaggagttcctgatcgg

SEQ ID NO: 400 - DNA, 396 bp, Homo sapiens
aactaaagaaagccacaaaagttcacctcaatgccaagacatttcttgatttttgaaaacccagttgtcgaaccacccatctatagaaacttgaaagactaaaaactatcttactctaaacattttctaggaagttgattctacaacacattttggttttccaatttggcttctaataattatttcaaagtttctgtgrcctaaattttgttttacattgatcctttgaatggactactgtttccacattttagaacatttaaaaagatatctacaacccgagtctaatcataaaaaaaatcagacagatccaaaatgtggaacattccactaaaaaaggagtggggagaggtctttattcttccaaaaatatcaatgccataaaagacaaagacg

SEQ ID NO: 401 - DNA, 396 bp, Homo sapiens
acccttcaaccccagcccagctgctaactgactacagccacatgaacagaaccaggtgagaccagaggaaacttccagtcacctaccagatcatgacaaataataaacgatgttttttaaaccacaaagatttggagcagcatttgttacacaaaattagacaactattacagttcgactaaaaacatgttcatttacratactaaattagaagtgtaagaatgggagaaaaacttcatactttaaaagtcattttttcctccaaaaacttccaactttgaaaaactgatttttataatgcataaaaattaaaataaccttagaatttatatgagtagcatagccagctggctttattatctgttgtactcaacacttcaataatcactgatgttt

SEQ ID NO: 402 - DNA, 396 bp, Homo sapiens
atgaccttacctcgttttgttttccttgtctgagagaaacacattagcagtctcccatcttgtttttccttttcctgtcacccaggacagagggcagtggtgtgatcacagctctgcagcacgacttccccaggttcaggtgatcctcccacctcagcctcccaaggagctgggaccacaggcacatgccaccacgtcsagcttaattttgtatttttttggtagagatcaggttttgccttattgccccaagctgatcttgaattcctgggctgaagcaatctgcctgccctggcctctccaagtgttaggattacaggtataagccaccgtgcagccttatattttgttttaaattttcctctgtatttttctctctggcaaattgtttaggga

SEQ ID NO: 403 - DNA, 396 bp, Homo sapiens
ttttttggtagagatcaggttttgccttattgccccaagctgatcttgaattcctgggctgaagcaatctgcctgccctggcctctccaagtgttaggattacaggtataagccaccgtgcagccttatattttgttttaaattttcctctgtatttttctctctggcaaattgtttagggagtttctttagtttatcrgactaaatttcaaggctttccttccaattttgacatgtaaacagtccctcatttctgcttatctagtgattattcccaaatctgtgtttacagtctagctgtctctcctgagattaagacttgtttctctaactacctgacggcagaatctcctcttggaagtatcaaggaggcagttcaaaactgaactgggcatt

SEQ ID NO: 404 - DNA, 396 bp, Homo sapiens
gctgatcttgaattcctgggctgaagcaatctgcctgccctggcctctccaagtgttaggattacaggtataagccaccgtgcagccttatattttgttttaaattttcctctgtatttttctctctggcaaattgtttagggagtttctttagtttatcagactaaatttcaaggctttccttccaattttgacatgyaaacagtccctcatttctgcttatctagtgattattcccaaatctgtgtttacagtctagctgtctctcctgagattaagacttgtttctctaactacctgacggcagaatctcctcttggaagtatcaaggaggcagttcaaaactgaactgggcattggctccactccttctccttctctttactattaataccc

SEQ ID NO: 405 - DNA, 396 bp, Homo sapiens
taagtcttatttaggcatcgtttcttctgggagacctttgtagaatctctgaggttatgttaacatgctaaggttttcttgacattctcagattgggttaggtgaacttttagcaacttatctttttactaaaaagtcatccctcagtatctgtggggaattggttctaggactccctaaggatatcaaaatctgcatragcagcccaggtgagaccagcagaagcactttacagtcacctacaggatcatgacaaataataaatcatgtttaagccacaaagtcctttacataaaatggtatagtatttgcatataacctacacatcttcctgtatcctttaaatcatctctagtttataatacctcatacgatgaaaatactacgtaaatagtt

SEQ ID NO: 406 - DNA, 396 bp, Homo sapiens
aagcagttcctaattactggacattctcagatctgctagagctacatgtccaattacgagaatatactggaaaaagccctggattagaaatgagaggatgtaggttttagtaccaggtcagccaccttgttaatgcaaatttgagtaaattgttacttcttttaggccttgtttttgctgttttgtttttctgacagtmtggtctctgtggtccaggctggagtgcagaggcacaatatcaggtccctgcagtctctacctcccaggatcaagccattttcatgcctcatcctcctgagtagctgggattacaggcatgtgccaccacaccctcgaactcctgacctcaagtgatctgcttgcctcagcctcccaaagtgctgggattagaggtgt

SEQ ID NO: 407 - DNA, 396 bp, Homo sapiens
gaatatactggaaaaagccctggattagaaatgagaggatgtaggttttagtaccaggtcagccaccttgttaatgcaaatttgagtaaattgttacttcttttaggccttgtttttgctgttttgtttttctgacagtatggtctctgtggtccaggctggagtgcagaggcacaatatcaggtccctgcagtctctrcctcccaggatcaagccattttcatgcctcatcctcctgagtagctgggattacaggcatgtgccaccacaccctcgaactcctgacctcaagtgatctgcttgcctcagcctcccaaagtgctgggattagaggtgtgagccactgtgcctagccttacacattgttttcttactggtaaagtgggaatatctaga

SEQ ID NO: 408 - DNA, 396 bp, Homo sapiens
gttttgtttttctgacagtatggtctctgtggtccaggctggagtgcagaggcacaatatcaggtccctgcagtctctacctcccaggatcaagccattttcatgcctcatcctcctgagtagctgggattacaggcatgtgccaccacaccctcgaactcctgacctcaagtgatctgcttgcctcagcctcccaaaktgctgggattagaggtgtgagccactgtgcctagccttacacattgttttcttactggtaaagtgggaatatctagaagttgcatgctacataaattcaaccatatattattggcaaaaaattttaaagaaaaacatcagcttaagagtactaattgagtacatgccttggaatgagcatgagctggaaagaacaaa

SEQ ID NO: 409 - DNA, 396 bp, Homo sapiens
ggcaaaaaattttaaagaaaaacatcagcttaagagtactaattgagtacatgccttggaatgagcatgagctggaaagaacaaacctgttgttacatcactcattgctgttttcatatgctgctcattgtaaatcttgctcagtggcatgattttagtgtttaaagatttatttgtttgtttgtttaggacaaagtcyctacacataatctacttgcttcatatatacatacttatgcatattatgtatgtacatacatgctctcagggctcacatgaaaaaacagccattcaggtgatgtgatttatctcatatgcttactttagagtcaacagggtgttgactccactatacaatactggcatggagaacacataagtcaaagtagacaggac

SEQ ID NO: 410 - DNA, 396 bp, Homo sapiens
tttatttgtttgtttgtttaggacaaagtctctacacataatctacttgcttcatatatacatacttatgcatattatgtatgtacatacatgctctcagggctcacatgaaaaaacagccattcaggtgatgtgatttatctcatatgcttactttagagtcaacagggtgttgactccactatacaatactggcatrgagaacacataagtcaaagtagacaggacccagccgtaccattggctagggcacaaatatattcacatatgtggagaatgatgtacgtagaaaggtcttcattgcacaatgctctttaataaagatctggaaaaaaaaaacacctaaatgttcaaaaggatagggtagatgaaataatggtacattataaaatggaa

SEQ ID NO: 411 - DNA, 396 bp, Homo sapiens
tctgtcacccaggctggagtgcagtggcatgatcatgtctccttgcagccttgacttccctggctcaggtgggcctcccacctcagtctcccaagtagctggaactacagtcgtgcaccaccatagccagctaagatagtgagatggtggccccactgtcttgcccaggctggactcgatttcctgggtgcaagcaccsttcccgcctcagcctcccaaagtgctgggattacaggcatgagtcaccattccagcctacttgtctttaattcttaaaaatattaatgttgagttttgtctcccagcatgtgggaaagatgtcatccattgcttctgtttcctggaggcctgggagcaaggagcccaggaacagtatcacgaagcttgagataatac

SEQ ID NO: 412 - DNA, 396 bp, Homo sapiens
atcattgatgggcatttgggttggttccaagtctttgctattgtgatttttttttttttttttttttttttaagacagagcctcactctgttgcccaggctggagtgcgatggcatgatctcagctcactgcaacctccgcctctcaggttcaagcaattcttctgcctcagcctcccaagtagctgggactacaggcrcccaccaccaggcccagctaatttttgtatttttagtagagacagggtttcaccatgttggtcaggctggtcttgaactccagacctcatgatctgcctgccttggcctcccaaagtgctgaaattacaggtgtgagccaccatacctggcctaggcagtctttttcaaaactctaagactgtgcttgtgtctcagg

SEQ ID NO: 413 - DNA, 395 bp, Homo sapiens
ggtatgaggtaaggatccatttttttcccatttgcatagccagtttttgtagctccactttattttctcacttgatctgccatgccacctctagcatgtatcaacatatcatgtatgtgtgcagctgttccttaactctcaattttattctcttggttactttgtctaacccagcactcatactttttaaattattayggctaccttgtagggcaagaatcctcacttttattcaacttcttttgaagtgtcttgatgcatattttttctgatcttacttggccatatatattttggggacagatgtgacatcataccaagctttctttgcttgacattgtagatattttcttattcattaatgtgctaaaaattttgagtttggtcatacagtc

SEQ ID NO: 414 - DNA, 396 bp, Homo sapiens
gtttctaacattatagacactagttttaggctcttggaggctagcagcaattctcagaggtaatgcaagcttccccatttcttcccgtagtcctgtgaaagaccagccacctccagaagcctacacatgagtcttctcagccatactttctgcttttcctaatgcctctcagcagcgtattagaaaggccatgatcgaygtacctgttaccttcaggctttgcataaggtgtatatgaaacataatgaatttcgtgtttaggctcaggtcccatccccaggttacctctttatcttggagacacttctggtcccatacatttcagataagagatattcaacctgtacccaccacgtaaggagaggaataggttttagaagaggagtcagggaggca

SEQ ID NO: 415 - DNA, 396 bp, Homo sapiens
gcatctattaaaagtgatggttttagtatcctgtctcattttttcctttccttacatcatgtattataggtaaacacatgcgcatgtgtgtatttctcttttagacaaaggatgagattactactgttagctcagtttttttttccctacttaacatctttgcttttattttttagacatatttctaagactattaaayattagacttacgtagcccttctgtcattgtgaaatacatagtttactaacagctaccatcaagataaagcctttatttaaataattaaacttcttagtggaaagctaagtaagcacagtttatggattttgggaatttttgccttgcatttgtctgatatggtaaaatattgagtttgtttttctcataatgttcac

SEQ ID NO: 416 - DNA, 396 bp, Homo sapiens
gataactcaatccccttaaagggttgtatcaagccattgataagggctcactttgatataaccattttctgttatttagacactctttcacacttcctattttcctcctggggatggtttgaatggatgacacaataccatattataaaagcactttacaaactgtaacttatgttataaatgtaattattaccttaargttttaccctgtttcagatttgagtggaagtagttctttacaatacaaaacaacttattttaactttttttgcatttcaaagaatgatcaatccacttcaggtgcagcatggtttccaaccctgacagcatggaagaatcatttatttagcttctaaaaatgtgcaggctgtaccctagaccagccttggggattag

SEQ ID NO: 417 - DNA, 396 bp, Homo sapiens
tcctctctctcattctctctctctctctctttctctctctccttctttgctccttcattccttctctctctctcttttttttttgagacagcatctcactatattgcccaggctgttctcaaactcctgggctcaagtgatcctcctgcctcagcttcctgagtagctaggactacaggcacatgctatggcaatactrttttaaacattgttttcaaggctccccaggtgattccagtgtgggtcatgtggtagagaaccactgacacaggcaaacaaaggatacataaagttgtctatttaatgggtaggtgcaggtagtagataagagtgtagccacataaaccacatgcttagtgaacggttttgttttgtgtgtatgtgagggattagcat

SEQ ID NO: 418 - DNA, 396 bp, Homo sapiens
ttcaggttccatttagcacgacagcagggaagggactgttggcagaaaaaaactggggcagtgggattaaagacagaccacacattccaaaaggcaccgtgggagggtcagggggcgaggttaggtctaggcttcagtgtcctgggagactcagtcttcacagggtgacagcgatcaagagtgcagcttaggctgggtrcagtggctcatgcctgtagtcccagcactttgggaggccgagacgggaggattgcttgaagccaggagtttgagaccagtctgaccaacatggcaaaaccccatctctactaaaaatacaaaaatcaactgggcatggtggcgtgtgcctgtagtcccagctacttgagaggctgaggcaagagaatcacttgaacc

SEQ ID NO: 419 - DNA, 396 bp, Homo sapiens
taaatgatcattatgttcatattcacacatacaataatgtactcaagtttattgctaaggtaattcagaatctccttattttgaagtgtgcatttgatatacctgtttgggaataactagtttcttatctttgacagaaaataattttgttgttttgtttttactaaaaaagcatggtgaaaaatggctccatttctawgagaggtaactaaaatatcgcaatttgctgggtgtcattaaagtaactcacaagggaaaaaatgcaaattggtatctgctgatggagtaaatctccgcagaagtgatgaccctgaaaggatcaatatattaaagcccctcccagctggtcattccagattgcaacaataaagcattaagtgttaaaacctcaaggca

SEQ ID NO: 420 - DNA, 396 bp, Homo sapiens
ctcatcaagcccacctttatacttcatttctccagacttcatgtccagactgtgggatgaacaagtggttataaggttttagaggctcctgtaggactagatggaaggcaaaaaaaggaaataacctttaagcatgctctcgattccttaaatcccatctgaaagtcttaaggatgtcttctcagtcatacttatttgrcaatattacctaattttctccattagcccaagctcaggggtctttcttcttccatattcacatgggtgcaatggttttctgaaaggaaaacagcattactagggcagtaacatttaattaatcacaggtacttatcaaactacaaaacaggcattccaggaactgggtgtttctgtttgtaaaattacactctcgtg

SEQ ID NO: 421 - DNA, 396 bp, Homo sapiens
taggactagatggaaggcaaaaaaaggaaataacctttaagcatgctctcgattccttaaatcccatctgaaagtcttaaggatgtcttctcagtcatacttatttgacaatattacctaattttctccattagcccaagctcaggggtctttcttcttccatattcacatgggtgcaatggttttctgaaaggaaaayagcattactagggcagtaacatttaattaatcacaggtacttatcaaactacaaaacaggcattccaggaactgggtgtttctgtttgtaaaattacactctcgtgtacatgctcccactaaaatgtaagttcgctgaggatggaggttttggtctctttgctctgtgctgtaaccccaacactgcagcagggcctg

SEQ ID NO: 422 - DNA, 396 bp, Homo sapiens
gctgcatagtctcacttaggtgtggaatctaaaaaagtcaaattaaaaaaaaatgtcaagcagagaatagaatggtagttgccagggactctgggaagtagcaggggtgggggtggaggggaggggatgggcagaagttggtcaaaaggtacaaagtttcaggtagacaggtgtaagttctggggatctattgtacagmgtggtgactgtagttaatactgtattgtgtacttaaaaattgctcaccaaaaatgttctcaccaaaaaaatgatgtttggatatgttaaacagtttgatttaatcattttgacgtgtgtgtgtgtgtgtgtgtgtgtgtgtgtgtgtatacatcaaaacatcacattatataccatatacaattaatatatacaatt

SEQ ID NO: 423 - DNA, 396 bp, Homo sapiens
ggggtaaatgctgactgcctgttctctggacaggaatggagaagatggtgctagcagggttgctgttcatatgtagacattcatgcagtcactctcttttcagcacacttcttacttctgccctgggttcagttgctgactctgagcccagaaaccttctagggttctgttaggtagattggcttccaccgtctttgcracaaccacagaaaattctagactgttttctcttcgggcttcattagtcaacttgcttcagtctgtcttgcatcttctaaatatttatagatctctctcttttgttggagtggcagaaaatgctagttgaccacccaatattcaaattatcctgcctccttaataacagaatatcattggatgtggtgggtaaataat

SEQ ID NO: 424 - DNA, 396 bp, Homo sapiens
atggagaagatggtgctagcagggttgctgttcatatgtagacattcatgcagtcactctcttttcagcacacttcttacttctgccctgggttcagttgctgactctgagcccagaaaccttctagggttctgttaggtagattggcttccaccgtctttgcgacaaccacagaaaattctagactgttttctcttcrggcttcattagtcaacttgcttcagtctgtcttgcatcttctaaatatttatagatctctctcttttgttggagtggcagaaaatgctagttgaccacccaatattcaaattatcctgcctccttaataacagaatatcattggatgtggtgggtaaataatataccctaactttccttgcagagaggggtggccaa

SEQ ID NO: 425 - DNA, 396 bp, Homo sapiens
cagggttgctgttcatatgtagacattcatgcagtcactctcttttcagcacacttcttacttctgccctgggttcagttgctgactctgagcccagaaaccttctagggttctgttaggtagattggcttccaccgtctttgcgacaaccacagaaaattctagactgttttctcttcgggcttcattagtcaacttkcttcagtctgtcttgcatcttctaaatatttatagatctctctcttttgttggagtggcagaaaatgctagttgaccacccaatattcaaattatcctgcctccttaataacagaatatcattggatgtggtgggtaaataatataccctaactttccttgcagagaggggtggccaatgagatggaaatgaaagtc

SEQ ID NO: 426 - DNA, 396 bp, Homo sapiens
tgggattgagttcttgatttgattttgagcttggccatcattggtgtatagcagtgctagtgatttgtgtacattgattttgtaacctaacactactaaattcacttatcaaatctgggagatttttgaggattccttaggattttctaggtatgagatcatatcattggtagaggtagtttgagtttctcttttccartttggatgccctttatttctttctcttgcctgattgctctgactagggcttctagtactatgttgaatagaaatggtgaaaagtgggcatccttgtctcattctaatttttagggggaaatgctttcaacttttccccattcattttgatgttggctgtgagtttgtcatagatgattcttactattttgagatata

SEQ ID NO: 427 - DNA, 396 bp, Homo sapiens
tcttttgccctgcctttctgcctttctgtccttttaatttgcgggcttttggcaaccacagcacgggtctggtttcctaggagtttcttttgtaggatcaaaccgctagttggctcttggccctgtgatagggccctgggctaacttattgggaaaatgttgctgtaacccctgcccagaggtgcctgtgacatgggcygccatcttctcctcttcccttggcttcagccccacctagaaacctgaacaaacattttccttgacatttcataaagtgtcagtggctcctcatttagcaaaatacatcccagggaagttcaaaagtgaaaaaaggccgtaacttcttcttcttctcagggacctacagaaaatatgtggcacctcggcagcctggcc

SEQ ID NO: 428 - DNA, 396 bp, Homo sapiens
catggattttgttttccaagtggcaagatggcgcctccacctttggtatcctattttagttcctggcagaaagaaaggaacaggctaatggccctgatgagtctacccccttttaacaggagaaaatttaaaaaacaaaaaccatgaaaccctttcccagaggcaacaaccagaattccatttatctttcattgaccaraacagaccacatggtcactggtggtggcaatggagactggggagatgaatatttttaaggtggcatattccagaagaacactgtgcactgattgcattaatgaacccattaatgtgccaaggggaggtttacctatgagcatgggcaaattagaacccactcttggagctgcaggtgagccaatcccacctaaacag

SEQ ID NO: 429 - DNA, 396 bp, Homo sapiens
tggtggtggcaatggagactggggagatgaatatttttaaggtggcatattccagaagaacactgtgcactgattgcattaatgaacccattaatgtgccaaggggaggtttacctatgagcatgggcaaattagaacccactcttggagctgcaggtgagccaatcccacctaaacagtgtggatgctacaagatggrgaagtaaattgattctattccataccctaacctctctccaagatgtattcttaaaatagaagagggaagacagaagaaaacatccagaatatatttttattgtcttttacttcttcagtgcattttagatcagtgcttctcaatctggcaaggggcatgcaggaggatgtgagttttatcaggaaaactacacaacc

SEQ ID NO: 430 - DNA, 396 bp, Homo sapiens
tgagccaatcccacctaaacagtgtggatgctacaagatggggaagtaaattgattctattccataccctaacctctctccaagatgtattcttaaaatagaagagggaagacagaagaaaacatccagaatatatttttattgtcttttacttcttcagtgcattttagatcagtgcttctcaatctggcaaggggcrtgcaggaggatgtgagttttatcaggaaaactacacaaccccccaaccacaatgctacccccactcctgtggaccttctttaagagagactcactattatagatggagttgatacgattttaagagaggccatatattatttgctttctgtcttgaaaaacttgtgatttttctgtattgtgctactgccaaagaga

SEQ ID NO: 431 - DNA, 396 bp, Homo sapiens
gggttgcagtgagcagagatcacaccattgcactccagcctgggtggcagagcgagattctgtctaaaaaacaacaccgtatttggggcatgctgatactaaaaaattattcattgtttgtctgaaattaaaatttaaattgggggccctgtattttactgggcaacccatttgcaatatcagcaacaatctcttattsagaccactgattaagtgtgcaaaatttgaatctctgaacagtacctatgtccttgatatcttaaattaatgagtgtcttagacactcaaagcaggaggaagcattatggcagatgtttgagccccagagatgtccatgagcacagcatagagctcagagccttctttattatttgcttcacgacagagcaaaggact

SEQ ID NO: 432 - DNA, 396 bp, Homo sapiens
catttgcaatatcagcaacaatctcttattcagaccactgattaagtgtgcaaaatttgaatctctgaacagtacctatgtccttgatatcttaaattaatgagtgtcttagacactcaaagcaggaggaagcattatggcagatgtttgagccccagagatgtccatgagcacagcatagagctcagagccttctttrttatttgcttcacgacagagcaaaggactgcagcaggttgactgatataaaagttttaccatgtctcacagcaggcctttgctcaagtttccagtaaggatattgtatcatttcttgcctgcagtacttgtaaatccacttacactgcctgctgttgagtcatttgtttcgtcttgagtagcatgtcatccttgttc

SEQ ID NO: 433 - DNA, 396 bp, Homo sapiens
ttgcagttctcattgctggggagtctaaactggaataaaacacccactatctccatcaggcttgcactagagcccagctctagctggagagaaagaagctaacccgcacagacacaggactgtaggcagggagcatccgggggtatttgggtcctggctctgatgtgcctaaggccaacttctctctggccatgctggygtgcatgagctcactaatcttcctttttgccttccattttctccaatcctgacttagcaaaggttgggcaaaagagactctgtgtgagttcgagcaaagcctgagatgctggattttccaagatacgagaaggggctgggggctgggtgaactggtggtggaggagggaaggattaatttcccaaggaggggaaggg

SEQ ID NO: 434 - DNA, 396 bp, Homo sapiens
gagaaagaagctaacccgcacagacacaggactgtaggcagggagcatccgggggtatttgggtcctggctctgatgtgcctaaggccaacttctctctggccatgctggcgtgcatgagctcactaatcttcctttttgccttccattttctccaatcctgacttagcaaaggttgggcaaaagagactctgtgtgarttcgagcaaagcctgagatgctggattttccaagatacgagaaggggctgggggctgggtgaactggtggtggaggagggaaggattaatttcccaaggaggggaaggggccaggacatcaggccccggggactttgaagagagggtcgtgggtaggaggtagatcaagtggagtgacacaaaggtcaggaaagagg

SEQ ID NO: 435 - DNA, 396 bp, Homo sapiens
catgcctcctacaaatttgacctgggcccagggccatgttcggtggtttttaagaaccgaggctcccagaagcagtattgggcagctagagtggccccaggatctatatcaaactctacctgtttctgaaccaaatttcttctagaattttattccataaatctgaattatggtgtcagactcctagcatacactaaakgaactctctgccttgcattaaataacaggagttacccctggaggtaactcctagccctggctctttagagaacagatgccgaataggcattaggggatgtgatggatgtgctaactttcaaaaaaaaaaaaaaaaaaaggcctgagctgagtgctcagagattcacaaaaagctgacagcatctctctgttccattg

SEQ ID NO: 436 - DNA, 396 bp, Homo sapiens
ctttggagcctggcagcctggctttgagaaccgggctttaacttgtcacatgactatggccaagttcctggggctctccaagcttcacttcctctgtaaaaagggcaataatataatacctgtcttattgggttttgtccatgttagatgagacattgggtacaaagcacttggtcccgtgcctggcacatttactgcrcttaatgtatgatagttttcttattattctaataaacaatatggctttgggagtatagttctgccacattgcagtggccagagtgaaggtggtgagtgccttctggggccctgggagtcaaggttatccgcatgccctttcttgcttgctcctcagtgtggctgcctctatgtccacaccatgcagatgcaacaggt

SEQ ID NO: 437 - DNA, 396 bp, Homo sapiens
acatgatcatccccttgggcttctggttttttttctttcaggaccttattttcaggcaagtggcctttgacctctaaggctgtcctttcctagctaccgaatccagcattcaaagtgatggaaatatgtatatatagtaatagtaaaatatcagcacttaatggcctgataagaatgtcactgcaatgctgagtttggrccaacatttgcctgctcctgccattgagcccgggctcccctccagagctgagctgctgcaagggatctgagtaactagggctgtgtcagagtggcgatgacagccaccacatgctaaggaagagatccccaaggacaaggagaatcccacgtggagctacttgcttctttgtcagtcttgtttttcttatttcacaa

SEQ ID NO: 438 - DNA, 396 bp, Homo sapiens
ccgaatccagcattcaaagtgatggaaatatgtatatatagtaatagtaaaatatcagcacttaatggcctgataagaatgtcactgcaatgctgagtttggaccaacatttgcctgctcctgccattgagcccgggctcccctccagagctgagctgctgcaagggatctgagtaactagggctgtgtcagagtggcratgacagccaccacatgctaaggaagagatccccaaggacaaggagaatcccacgtggagctacttgcttctttgtcagtcttgtttttcttatttcacaaccttctaaaacacaatctctcaacctctattgttagcttgcatttttcaatcatgagcacagctttacctggctccatgctttgattgactctacc

SEQ ID NO: 439 - DNA, 396 bp, Homo sapiens
tcttatttcacaaccttctaaaacacaatctctcaacctctattgttagcttgcatttttcaatcatgagcacagctttacctggctccatgctttgattgactctacctgccaacactgcaacaacagggaaagggacaccggcctcataccattagatggtgtgtagcctgggcatgaggataattaaaaactcccwaggggattttaacatgtaacacagtttggaaaccattgatgtaagatcttcttactcaacatgtgctccaaggagctgttgtatcagcttatcagaaatgtagatcaggccgcacttggacctgtagaatcagaatctgcattttatcagattccgacattatttgtatgaacattagcttttgagaagtgttgctt

SEQ ID NO: 440 - DNA, 396 bp, Homo sapiens
cttttgacaccaactacaagtcaaggggttccccaaaccaccctgagttgtgataattcgctgggagatctgacagaactcactgaaggttgttatactcatggttgtgatctcttatagggagggaatacagattaaaatcagccaaaggaagaagcacacagcacagagtccaggacagtgcctgacatggagcccytacggtcctctcccgtggagtcacggacagcgccactctcctggcattgatgtgtgacaacacacagggagtgttccccaccagggaagccttggtgtccagggtctttactgtggctctgtcacatgagcacagctgactgcccatgcggccgatctgttcccagactctccaccgctacacatcactcacagtcc

SEQ ID NO: 441 - DNA, 396 bp, Homo sapiens
gtggctcacagaactcagggaaacacagctaccagtttattgcgaaggacattttaaaggataaaagtaggcagataaagagatgcatagggcgaggtgtggaaaggtccctagtgcaggagcttctgtccatgtggagcgggggtgcaccaccctctcagtacatgaatgagttctccttcacctgcctatcagcctytacatgttcagctccccaacccagtcctcttgggtttttatggaagcttcaagacacccacattctttccccagagtatagggcaagaccttctctggggagggttttaagacccacagtcagaaaggtggggtggggtcaagattagagtcctgccttgacgggcaggtgaaaggggtagggggagtaggtgagaa

SEQ ID NO: 442 - DNA, 396 bp, Homo sapiens
cgggggtgcaccaccctctcagtacatgaatgagttctccttcacctgcctatcagcctctacatgttcagctccccaacccagtcctcttgggtttttatggaagcttcaagacacccacattctttccccagagtatagggcaagaccttctctggggagggttttaagacccacagtcagaaaggtggggtggggkcaagattagagtcctgccttgacgggcaggtgaaaggggtagggggagtaggtgagaaaaattctgtttattttttctttttttttttgagacggagtttcactcttgttgcccagggtggagtgcaatggcacaatctcagctcactgcaacctccgcctcccaggtttaagcgattctcctgcctcagcctcccg

SEQ ID NO: 443 - DNA, 396 bp, Homo sapiens
atgagttctccttcacctgcctatcagcctctacatgttcagctccccaacccagtcctcttgggtttttatggaagcttcaagacacccacattctttccccagagtatagggcaagaccttctctggggagggttttaagacccacagtcagaaaggtggggtggggtcaagattagagtcctgccttgacgggcargtgaaaggggtagggggagtaggtgagaaaaattctgtttattttttctttttttttttgagacggagtttcactcttgttgcccagggtggagtgcaatggcacaatctcagctcactgcaacctccgcctcccaggtttaagcgattctcctgcctcagcctcccgagtagctgggattacaggcgtgtgccacc

SEQ ID NO: 444 - DNA, 396 bp, Homo sapiens
tcttcattccacaaagctcagtgtcaaaacatggggtttacactggaagctgaggtcacatcagtagccgggatcagggtcgccctagctgcccaatgcagctcccaggcctcctgtaaaaccttgacctttgaggtcatgacagccctctcctgctatgctcatagctgaccactgaactcctggacactccctcccscaagttcacagagaatgtgggcacatgccttacagtcttcccttgatccaaactactgccttcatcttgagtgacagcagcatcttttggatgtcttggcctgtctagctttatttttttgtgttctgccatcaagttgctacttctgttgccatcgtgcctgtcagcgcagtgcaggctgtggtgaaatcccacga

SEQ ID NO: 445 - DNA, 396 bp, Homo sapiens
tatttttttgtgttctgccatcaagttgctacttctgttgccatcgtgcctgtcagcgcagtgcaggctgtggtgaaatcccacgaactcaggcatcacactgaccgggtctgagtcctgtctcagttgtcagctagttgtgcaatgaagggaaagggacctacactttccaagcctcaattcactcatctatggcatkgtgacaataatggaggttgatttaaagtcctttgtaagaattaagagttataatagacataaagtgctgtatctggtatacctagaaaacattccataaaagttagtaattgttggtcatgtaatgatgactctctaggctaggatttcagcttcattgcatgcacatggtgcactcacagggcgtgacctctctct

SEQ ID NO: 446 - DNA, 396 bp, Homo sapiens
ggtatacctagaaaacattccataaaagttagtaattgttggtcatgtaatgatgactctctaggctaggatttcagcttcattgcatgcacatggtgcactcacagggcgtgacctctctctgtctcagtaacctcatctgaggaccgggataatcataccgcttcaaagggatgtcataaagattaaataatatgtrtaaggctgcttgcatttagctgcattcaacaaatatttctgtatctttctcctcatttctccttactttcttgcttattatctgctctaggtatagatttcagagaactaagcttgttacaatccttcataaaataaccaggttggttagggcatttccaagagtcaatactgtttagtgactattctctgtttaat

SEQ ID NO: 447 - DNA, 396 bp, Homo sapiens
aaggctgcttgcatttagctgcattcaacaaatatttctgtatctttctcctcatttctccttactttcttgcttattatctgctctaggtatagatttcagagaactaagcttgttacaatccttcataaaataaccaggttggttagggcatttccaagagtcaatactgtttagtgactattctctgtttaatctmttttgattgtccagggtcatcttttgctatgtcataggttgttggcttcttctagagaagtgagacgatggacaagttccaagtgagtgaggcgactggtcaggatattccgctgaaaaactcatgtcagttctaattcgtgattgtaattcaatcacagcctgagaacagtaggactgtagttcaaatgctctgtt

SEQ ID NO: 448 - DNA, 396 bp, Homo sapiens
cctgggttcaagcaattctcctgcctcagcctcccaagtagctgggactacaggcacatgccaccacgcccagataattttcgtatttttagtagagacggggtttccccttgttggccagggtggtcttgatctcttgacctcatgatccgcccacctcggcctcccaaagtgctgggattacaggcgtgagccaccrcgcccggcctctagaggataatttttaaatgtgcttttgcatttggaaaatgtgattggcatttttttctaattttctaatatgatacgctgtcggatgctatggattacttaaaccctctggctacctagaaagatctttaagtggttctcaacaagcttcatacgcaatgtaaattgtattatctctcaggatgt

SEQ ID NO: 449 - DNA, 396 bp, Homo sapiens
tgtgattggcatttttttctaattttctaatatgatacgctgtcggatgctatggattacttaaaccctctggctacctagaaagatctttaagtggttctcaacaagcttcatacgcaatgtaaattgtattatctctcaggatgtgtgagaacatctgtttttcttctaatgcagtaaacatataagggtctcttgrgatatcttttaaatagacttaatacaacattcaggaatgataacaaaatataatcacagttgtaagggaatgtgagcatttcatattaataacattggaaccttatgtttaatacagtgttaaaagttgacaaacatgtaggagtcagaaaattcaattaaaattatcacagtaatatgaatttagccacatcctgt

SEQ ID NO: 450 - DNA, 396 bp, Homo sapiens
acttaaaccctctggctacctagaaagatctttaagtggttctcaacaagcttcatacgcaatgtaaattgtattatctctcaggatgtgtgagaacatctgtttttcttctaatgcagtaaacatataagggtctcttgggatatcttttaaatagacttaatacaacattcaggaatgataacaaaatataatcacrgttgtaagggaatgtgagcatttcatattaataacattggaaccttatgtttaatacagtgttaaaagttgacaaacatgtaggagtcagaaaattcaattaaaattatcacagtaatatgaatttagccacatcctgtgttagttatgaaatccatttaacaccacaaacagtaatatttttagccagtttattca

SEQ ID NO: 451 - DNA, 396 bp, Homo sapiens
catttaacaccacaaacagtaatatttttagccagtttattcaaaaggaaaacaggaactaaaccactttcatgcaatatatactctgttaatgtggtcaggctaattttgctgggggaaggaacttaacttttgaatatttgaatgcccagtcatttaatctgaatatcctatttccttgcatgttgcaaaatttttktcaataaaaggcagaaaaagaaatctcttctccatgctcatccctaagagaatgggttgtctgtaccctgagagcattttatggaggggacaaccacttttctaattttccttcccacttctctgtgggcacaaatgctctttggttgaaagagttgtaattcagtcccaagatgaggtgtggttactgcatcccta

SEQ ID NO: 452 - DNA, 396 bp, Homo sapiens
tcaatccatgctccacactgcagccagagtgctctacaatgcaaatccatttgtgagactcctcctcttaaaatcctcaagtggcttctctttgcccccaggatcattttgaaactccttaatggaagaggcatggccctttgggatgtggttccccaacccctcccacatcatcttttcaatcagatttcccactaartggaaattttttcaggtcctcaactttatggtgactttctcttgctcaggatctttgaacatactgtttcttctttccttttgtatttgccaagacaacacttcctctggtaagattttcctgacatcctctataaaaaaagattgagatagttgactacccaaaatgtttcccattcattccaagctctattcaag

SEQ ID NO: 453 - DNA, 396 bp, Homo sapiens
aacacttcctctggtaagattttcctgacatcctctataaaaaaagattgagatagttgactacccaaaatgtttcccattcattccaagctctattcaaggcagtaaagtgcccggctgacagattgcattcctcatcttttctgaagctagcaatggccatgcaacagcattctggccaataagatagaagtcgaarttgaagggtgggatttccaagaaagctcgttgaagacataattcctcatttcacttcttactctttctctttcctgcttcctaaaatgcggtgcagatggcagacacttcaaagctgtctcaggcaatcaggtgatgttaaggcagaaaccagctttatgatgggtagaacaggaagaaagaaggcacctatgttct

SEQ ID NO: 454 - DNA, 396 bp, Homo sapiens
cctacaaatctcatgttgacattttatccctaatattggaggcagggcctagtaggaggtgttttggtcatagtgataaatggcttggtgccgttctcacagtaacgagtgagtttttattctagtggttcctgcaagaactgattgttaaaagagcttggatccttccacccctctctcactcttgcttcctctctcwcaccttgtaatctctacaagctcttcacctccccttctccttttgccataagtggaagatttctgaggcctcaccagaagcagatgttggttccatgcttcttgtacagcctgcagaaccatgagccaaatcaacttcttttctttataattatccagtctcaggtattcctttatagcaacacaaatggactaaga

SEQ ID NO: 455 - DNA, 396 bp, Homo sapiens
gttgtttccagctttgaactattttgaatcctaaaagactgccagttttgaatgagaccccagaacaatgaatgtaggctctgtatacaagttcaggctgctgggcaacttaggccttaagacacaactctgccacttaggccttaagacacaactgacatgatggtgcttaaagtggctgtgatggaaaaggaggctrtttggagcctttggagtgcctttataggtgaaccccagcatagcacctaatgatttggagcaaagctgtgtcattccccaaagataactattcgccttttgagaaacatcttctagctactatcaataataaacacagaatgcatcaccatgggccaccgtgttgtcttttgacctgagtttccattgtgaacaaga

SEQ ID NO: 456 - DNA, 396 bp, Homo sapiens
aactctgccacttaggccttaagacacaactgacatgatggtgcttaaagtggctgtgatggaaaaggaggctgtttggagcctttggagtgcctttataggtgaaccccagcatagcacctaatgatttggagcaaagctgtgtcattccccaaagataactattcgccttttgagaaacatcttctagctactatcrataataaacacagaatgcatcaccatgggccaccgtgttgtcttttgacctgagtttccattgtgaacaagagtcatttgatccaaggcagaaagttgggtgcacacagcagtgttccatcatcaaatggaatatgagattgggcccaagtaggtcctgcagacacaaataagttgcaagagcaagtagtacaggcg

SEQ ID NO: 457 - DNA, 396 bp, Homo sapiens
gaaaaggaggctgtttggagcctttggagtgcctttataggtgaaccccagcatagcacctaatgatttggagcaaagctgtgtcattccccaaagataactattcgccttttgagaaacatcttctagctactatcaataataaacacagaatgcatcaccatgggccaccgtgttgtcttttgacctgagtttccaytgtgaacaagagtcatttgatccaaggcagaaagttgggtgcacacagcagtgttccatcatcaaatggaatatgagattgggcccaagtaggtcctgcagacacaaataagttgcaagagcaagtagtacaggcgcttggcctggccagtactgttgccaagttgactgcttcccctcagtctgcatctgtggctt

SEQ ID NO: 458 - DNA, 396 bp, Homo sapiens
ccccaaagataactattcgccttttgagaaacatcttctagctactatcaataataaacacagaatgcatcaccatgggccaccgtgttgtcttttgacctgagtttccattgtgaacaagagtcatttgatccaaggcagaaagttgggtgcacacagcagtgttccatcatcaaatggaatatgagattgggcccargtaggtcctgcagacacaaataagttgcaagagcaagtagtacaggcgcttggcctggccagtactgttgccaagttgactgcttcccctcagtctgcatctgtggcttcatggggagtttcctatgaccacttgatggaggaaaaaacaaattggagcatagtttatagtgctggtactacccaaagtggctagct

SEQ ID NO: 459 - DNA, 396 bp, Homo sapiens
gtccgtgagttacagatctacacaaaatcacagagagtggttaatcgtttagtctgatggtcagggacttccaagagacatgattagaaaactggtgacaaggagtcctggggaagaggcatatggatacctctgaacacacacaaaacatgagaatatgtatcccatatgaatgttaaccaaagagcagccacaacasaagaggattttaaaatcagctgaataagatgattcattctgacagcatcagctagtctctttccccagccactgttgcccagtgggcttacatatatcatggccatgggggcagggctatgtatggacacagcaacatgaatttccactcatcaaggccaatttggctccagccattgctgagtgctcagcctgcca

SEQ ID NO: 460 - DNA, 396 bp, Homo sapiens
acatgattagaaaactggtgacaaggagtcctggggaagaggcatatggatacctctgaacacacacaaaacatgagaatatgtatcccatatgaatgttaaccaaagagcagccacaacagaagaggattttaaaatcagctgaataagatgattcattctgacagcatcagctagtctctttccccagccactgttrcccagtgggcttacatatatcatggccatgggggcagggctatgtatggacacagcaacatgaatttccactcatcaaggccaatttggctccagccattgctgagtgctcagcctgccaagatagaaatctacgccaatatggcaccattccctgggctagaaaaccaactggtggaaggttgattacattggacc

SEQ ID NO: 461 - DNA, 396 bp, Homo sapiens
gggaatacaatggtggttccactaaactgacagctgagtttgccatctcctcgtgccagtgaatacacaagcaaggaagggggttcctttctcacctagggtgactgatcctaattaccaaggagaaattggactgccacttcacaatgagggtgaggagtatgtactctatgtgtctgtgattaatgtcaatagaaartgacaccaacctagtacacagaggactgatcatggtccaggcccttcaggaatgaagatttgagtcaccaggcaaggaacttggactcactgaggagggcatattccaaggagaatattttatctatgtccatctatgtccatctatattccatctgtgttccccttggaattcctattcatgaacatggggaattc

SEQ ID NO: 462 - DNA, 396 bp, Homo sapiens
tatagaatgagtagtggaaggtagttataaatgtaagtcaaaaaccacacaaccaatttgagaaatgaggaaggtaatagtgttgaatatgtcttctttatcttgatataaatgtatttgtgcatatattaaccagtttatttatttattattattttttgagatgagctctcgccatgttgcccaggctggtcttgamctcctgggctcaactgattctaccatttagtcctccgagtagctgggactacaggcatgcaccaccatacccagctgaccagttttttcctattcctctacttaatttctctactatacaacataatatgtgttaatggtagttaactttatatctcagtattaagtcacaagatatcaaaaagggaatgcgactta

SEQ ID NO: 463 - DNA, 396 bp, Homo sapiens
atgtcttctttatcttgatataaatgtatttgtgcatatattaaccagtttatttatttattattattttttgagatgagctctcgccatgttgcccaggctggtcttgaactcctgggctcaactgattctaccatttagtcctccgagtagctgggactacaggcatgcaccaccatacccagctgaccagtttttycctattcctctacttaatttctctactatacaacataatatgtgttaatggtagttaactttatatctcagtattaagtcacaagatatcaaaaagggaatgcgacttagttacaagcagaatgaatatcactcaaagatgaataaagagaagagggttagtgcattttctgttggatgagagaaagtttcattgtt

SEQ ID NO: 464 - DNA, 396 bp, Homo sapiens
gcagtggcgtgatcccagctcactgcaatctctgcctcctgggttcaagtgattctcctgcctcagcctcccgaggggctgggattgtaggcgtgcaccactatgcccatctaatttttgtatttttagtagagatagggttttgccattttggccagactgtcttgaactcctgacctcaggtgatctgcctgcctcrgcctcccacagttttgtgattataggcatgagccaccgtgcccggccttaacctttgttttcttacacaacacactacgtgatgttttccacatgcatgggtcatttgcttcatttacgtacaaatgcataagcaatatactgtgtggtgtgagtttgtgatgggaaaaggaagaagttttgcggatactacactgg

SEQ ID NO: 465 - DNA, 396 bp, Homo sapiens
gcccaggctgttctccaactcctggactcaagccatcctctagcctcggccttccaaagtgctgggactataggcgtgagccacggtgccaggcccttgaccacatttttaacccctctgaacctcagtttcactttctgggcaatgggaggggggtaatttgtccctcagagggttgcactgaggggcaaatgtgagsctctgggtacaatgcccagtacagactaggtccccacgacacagccgctcagcggctccggattctgggctgctctggactgcggccaggcggtcttctgcgggaatccgggcaggcagggcgggctgcgctcccctccccggctctcccggtgccccttgtctttttgttctgtctcagcagctctctattaagat

SEQ ID NO: 466 - DNA, 396 bp, Homo sapiens
tttttgttctgtctcagcagctctctattaagatgaatggcatttccaaaggcttcacctctgataagtgttcctctgcagctgcagccagaatcttaatgtgcgcgctgtaatttaatggccgtctcggctattaacacgctcttctcgggtgaagtggactccctccatccccgggcctctgcacgtgctctgcgcrctggctgggggtgactccaaggagctcagagcggggtgcccggcacctctcgccaggcgcctttcgaccttctaaagcgcgaatggctggacttttctcccatgtgtggggccccagaaggtgtggggccccagaaggtgtggggtccctgcgttccacggagcccggaaggtttccagtgatggtgggggctgacc

SEQ ID NO: 467 - DNA, 396 bp, Homo sapiens
ggagcccggaaggtttccagtgatggtgggggctgaccacgttggtccccgtgggtgctgttttcatgtgccggcagattgggatgagtttaaaagacagaagcgtgtaggatagagaaacttctttaaaaactggaaattttaatctggggattataactattggacagtcaagtgcaagagtgaatacacttctcastccctcctcccaatttttatttgcgggattagtcagtccccctctgccacatgataattgtgagaactaccagggtcttcattctcctgccatctggttgacctctccaagaatggacacccgggcagcctgggccaatgaggctgtcctaagagtttagatgagagaagtcagtctttgacaggtgatggaagctg

SEQ ID NO: 468 - DNA, 396 bp, Homo sapiens
cagtgatggtgggggctgaccacgttggtccccgtgggtgctgttttcatgtgccggcagattgggatgagtttaaaagacagaagcgtgtaggatagagaaacttctttaaaaactggaaattttaatctggggattataactattggacagtcaagtgcaagagtgaatacacttctcactccctcctcccaatttytatttgcgggattagtcagtccccctctgccacatgataattgtgagaactaccagggtcttcattctcctgccatctggttgacctctccaagaatggacacccgggcagcctgggccaatgaggctgtcctaagagtttagatgagagaagtcagtctttgacaggtgatggaagctgtaaaatgtaaaactcca

SEQ ID NO: 469 - DNA, 396 bp, Homo sapiens
taagagaagctgagagagagcgagaggagagattggaagaaagacagagacagaggtagagagaagggaaagagagagagaaagggacagaagagagagaaaaaagagggggccgggcgcggtggctcacgcctgtaatctcagcactttgggaggccgaggcgggcagatcacgaggtcaggagatcgagaccatccyggctaacacggtgaaacccccgtctctactaaaaaatataaaaaaaattagccaggcgtggtggtgggtgcctgtagtcccagctactgaggaggctgagacaggagaatggcgtgaacccgggaggcagagcttgcagtgagctgagatcgcgccactgcactccagcctgggcaacagagcaagactccgtctca

SEQ ID NO: 470 - DNA, 396 bp, Homo sapiens
tccaccagcagcttttctgagtctccagcttgcagatggcaaaccatgaaacttcatggtgtccatgagcatgtgaaccaatttctattataaatctgcaatatatatatatgaggagacttatttatatattggttcagtttctctggagagccttggctaatataaagtctatactctacaaagtgccctaggtackcagggagtacccaagtgtgtcatgaccagcccgacagccctggctgctggcttccccgcacacaactctgcacgctgccttcatcagcctttctctctcagctgaaccgagggcattgaagcgggcctctggcactgtacctatgagggagcaatatcttcccctacactgacctcttccgtgccgagatgcagccc

SEQ ID NO: 471 - DNA, 396 bp, Homo sapiens
gcctctggcactgtacctatgagggagcaatatcttcccctacactgacctcttccgtgccgagatgcagccctccctgctgccactagttacagtggtccatgttccctttcaaagtgaagttttgataaaagcacctcttaaccaatgccaaatagctaagtctgggacaaagattgcaggtattttgcattttccwtgtaacctcagagggattgccattcacactgatctgagctgcagaataccaggcagccacctcacccacccagcaggtccactcttatactttctcagaaagcacagccactctactcttattcagttgaaaagaatttccaggaaggtgtttctgcgattgcctcagaaaagtcagttccctttgggaatttccct

SEQ ID NO: 472 - DNA, 396 bp, Homo sapiens
tacttttctctgaagaaatggagatatcagctgtccctccccactgccatttattccttccttcattcaaaccttatgtggctgctacttaccgtgtgttaagtgttcactttttttcttggaattcaaaaaaagaaggacagtatttggggcacagatcttttggtgttctatacatttttttaaagtttcattttayatttgtgtgtgcgtgtgtgtgtgtgtgtgagacagtcttgctctgttgcccaggctggagtgcagtggcataatcattggctcactgtagcctcaaagtcctgggcccaagcaatcttcccacctcagccacccaaaatgctggggttacaggtttatgccactctgtctgacctgaaagttttgggtttactttcc

SEQ ID NO: 473 - DNA, 396 bp, Homo sapiens
gcataatcattggctcactgtagcctcaaagtcctgggcccaagcaatcttcccacctcagccacccaaaatgctggggttacaggtttatgccactctgtctgacctgaaagttttgggtttactttcccttctttctctttgctgaagtcagagatgatggcagcttccagattctctggtgcctgtgctgggctcrtgctggtcatggtcttgggtccaggattcattctggagactctcagggaagtttcccatgacaaggaaatgtaggagagtgtgctggctttgcgtgctcctctgccaagccctgcttctcctggtgggacacactgaaccacagccagggcattttggtggttagttaaaaaaaaaaaaaaaaaaaaaaaaaggaag

SEQ ID NO: 474 - DNA, 396 bp, Homo sapiens
cttcagaaattgtaatgatgaaagagtgcaagctctcacttccccttcctgtacagggcaggttgtgcagctggaggcagagcagtcctctctggggagcctgaagcaaacatggatcaagaaactgtaggcaatgttgtcctgttggccatcgtcaccctcatcagcgtggtccagaatggtaaggaaagcccttcamtcagggaagaacagaaggggagattttctttgatggttgtttggaagtcaggcttaaacaattgtgtctgtgtgtgcgcatgcacaaacacttttaccttatctttattttcttctttttatttgaatgtatagggttgtgtgtatttctgtgtaaatttggggttttcctcctcttagtctttcacttttgtggtg

SEQ ID NO: 475 - DNA, 396 bp, Homo sapiens
ttttctaacatctgcagtgcaattgaagttaccagtcatctgcagtctaaaaagaaagtgattttgggaggtgcgtagaaaaaatcatcttattatttttcctctatattacttttttctttttttctcctgaagaaacttttttttttggtgataccttctttttctctagcacgtataattttggaagcatttttcrtatgcagtgtatacttcagaaagagagagagagagaggaaaattgtcctgttcagcgtttgcatttccattattcctgctattagttaaaaacaacaacaacaacaaaaaacaagcaggatacctagatctggaaaagggagaattgtgtagagctgtcttcctaaagttctgagttagggctgcctcagaccactt

SEQ ID NO: 476 - DNA, 396 bp, Homo sapiens
ttttggaagcatttttcatatgcagtgtatacttcagaaagagagagagagagaggaaaattgtcctgttcagcgtttgcatttccattattcctgctattagttaaaaacaacaacaacaacaaaaaacaagcaggatacctagatctggaaaagggagaattgtgtagagctgtcttcctaaagttctgagttaggrctgcctcagaccactttcataactatctccagtggctttgtgttttatatttattaagatagagaaaaaaagagtaattactaagggcagctgctgtagctttatggtgattactgaacattgacatgctgtcacgtttttggaactttgagtatttaatcactttgggatattctattttcccccatcttgagtgt

SEQ ID NO: 477 - DNA, 396 bp, Homo sapiens
ggaactttgagtatttaatcactttgggatattctattttcccccatcttgagtgtggacagatgctggtgatgtagccttctgggcacagagcaagcctccccctcagcctctgcaccagaaaggctcagcttcacacactccaagtatgttttctacaagaactacactttgtggctttctgacccaaacatttttrtactaaattacacacaacaaagttgtagctcagagagggaacaaatggcttatttaggccaccattttcttgagccattatgatttcacacagggctcccttggccctgtaaattggcaaggattccattattcaacccgcatacatgtacagagaccctgctctggcccagatagtattctgggtacaggcggata

SEQ ID NO: 478 - DNA, 396 bp, Homo sapiens
tgtggacagatgctggtgatgtagccttctgggcacagagcaagcctccccctcagcctctgcaccagaaaggctcagcttcacacactccaagtatgttttctacaagaactacactttgtggctttctgacccaaacatttttatactaaattacacacaacaaagttgtagctcagagagggaacaaatggcttayttaggccaccattttcttgagccattatgatttcacacagggctcccttggccctgtaaattggcaaggattccattattcaacccgcatacatgtacagagaccctgctctggcccagatagtattctgggtacaggcggatagagcaggaaacaaaacagctacagtgatggacaggtcagcctgcagcaatgcc

SEQ ID NO: 479 - DNA, 396 bp, Homo sapiens
tttttatactaaattacacacaacaaagttgtagctcagagagggaacaaatggcttatttaggccaccattttcttgagccattatgatttcacacagggctcccttggccctgtaaattggcaaggattccattattcaacccgcatacatgtacagagaccctgctctggcccagatagtattctgggtacaggcrgatagagcaggaaacaaaacagctacagtgatggacaggtcagcctgcagcaatgcctgcagtctctgcaaaggtagctgtatgggtgggcaggtggctagcacttattcagctctggaaggatctcccctctggcctctcccctgacacccatcaataaaactgaggagcatcggtggacaggggaccttgtgccc

SEQ ID NO: 480 - DNA, 396 bp, Homo sapiens
ttttcttgagccattatgatttcacacagggctcccttggccctgtaaattggcaaggattccattattcaacccgcatacatgtacagagaccctgctctggcccagatagtattctgggtacaggcggatagagcaggaaacaaaacagctacagtgatggacaggtcagcctgcagcaatgcctgcagtctctgcraaggtagctgtatgggtgggcaggtggctagcacttattcagctctggaaggatctcccctctggcctctcccctgacacccatcaataaaactgaggagcatcggtggacaggggaccttgtgccccctccctgcctgtgcagttggggctgaacccagctacgaagtttgagctcactctctccagctccctctc

SEQ ID NO: 481 - DNA, 396 bp, Homo sapiens
gacaggtcagcctgcagcaatgcctgcagtctctgcaaaggtagctgtatgggtgggcaggtggctagcacttattcagctctggaaggatctcccctctggcctctcccctgacacccatcaataaaactgaggagcatcggtggacaggggaccttgtgccccctccctgcctgtgcagttggggctgaacccagcyacgaagtttgagctcactctctccagctccctctcaattcagagctgaactgtgggaagcttcagagctctctgtttcaaggacaggttctcctcacctctcctaatggaggtgcaccagggaactggccctgctctgcccagggctttctcctggactttgccatcatggtctagcaaaccctgttcagattgagg

SEQ ID NO: 482 - DNA, 396 bp, Homo sapiens
cactctctccagctccctctcaattcagagctgaactgtgggaagcttcagagctctctgtttcaaggacaggttctcctcacctctcctaatggaggtgcaccagggaactggccctgctctgcccagggctttctcctggactttgccatcatggtctagcaaaccctgttcagattgaggtgagtggtgagatttygaattctttttgacagataggattaagtcttcttctgtgggacaagtgggaggtagaggtaagattaaagatggccaaatgtctgagtcctgacagccacaatatggagatctagactttttacagaccacagggcacaggggcctcactaacagagttcccggaagtgatgagtgtgctgggggcttcctggttga

SEQ ID NO: 483 - DNA, 396 bp, Homo sapiens
taggattaagtcttcttctgtgggacaagtgggaggtagaggtaagattaaagatggccaaatgtctgagtcctgacagccacaatatggagatctagactttttacagaccacagggcacaggggcctcactaacagagttcccggaagtgatgagtgtgctgggggcttcctggttgaagagacactagaatggacsagctgggagctaattttttgggctggagtgtgatggcctgcacatcactgcctctgtccctccattgtcacagctgccccttaggagccagctgaggcaatttgtggtcagagtgactttgcacagttgtcctgcctgtgttcaggaagggagtttctgtggtccctttgaaaccacagaagagcccctcgtatagc

SEQ ID NO: 484 - DNA, 396 bp, Homo sapiens
agttgtcctgcctgtgttcaggaagggagtttctgtggtccctttgaaaccacagaagagcccctcgtatagctctcaatggagggggcaaaacattcaaataactcaggagataacacaactatttgtttttaactgtgagtttttaggcaatcacaaagatccagatgtatgtccaagcctctctttgcaattctawttaacctcaatgttgcaaccatagacctaccttacagagttcaaaaaaatatgcaaaaaccctgcctttcttcttcctcataccccaaaatgccattctgaacatttcctgttagttaaaaaaagatttccatggtgttaccaggcactgtacacagtctgtgtcccaagacaaggaggtacagttccacatgcgcc

SEQ ID NO: 485 - DNA, 396 bp, Homo sapiens
agggggcaaaacattcaaataactcaggagataacacaactatttgtttttaactgtgagtttttaggcaatcacaaagatccagatgtatgtccaagcctctctttgcaattctaattaacctcaatgttgcaaccatagacctaccttacagagttcaaaaaaatatgcaaaaaccctgcctttcttcttcctcatwccccaaaatgccattctgaacatttcctgttagttaaaaaaagatttccatggtgttaccaggcactgtacacagtctgtgtcccaagacaaggaggtacagttccacatgcgcccatgactgggttgggctctgcactctctctatactttgagagcctgattttctgtgattgggcagagctggcccacctggtg

SEQ ID NO: 486 - DNA, 396 bp, Homo sapiens
tctgcactctctctatactttgagagcctgattttctgtgattgggcagagctggcccacctggtgcaatgtcctcctctgcctttcaaacatgttttagtcatcaagatcttcaaatttgtaaccctttccagcttgatccagcagaatgcagatttggaaaaacagaacgagtttaaaatacatgattctaagaaayctggaccagaactatcaaaacttggtttcccagagaatatagcaaatgggctcattggccaatactatgacattggcttttgagaaaagaaaggctttattgcaaggctggccagcaaggagacaggagttgggctcaaatctgtctccccagtttggggcttagggcaagttttaattacacagacgcatttctta

SEQ ID NO: 487 - DNA, 396 bp, Homo sapiens
aaccctttccagcttgatccagcagaatgcagatttggaaaaacagaacgagtttaaaatacatgattctaagaaacctggaccagaactatcaaaacttggtttcccagagaatatagcaaatgggctcattggccaatactatgacattggcttttgagaaaagaaaggctttattgcaaggctggccagcaaggaracaggagttgggctcaaatctgtctccccagtttggggcttagggcaagttttaattacacagacgcatttcttatgagtagcaggcagagagcctccaacttcttctgcctaggtaccagcagcttagacatgatgcaaacctgggaagcacatactgtatttggagaaagtgattgggaagaaatgtgagctgag

SEQ ID NO: 488 - DNA, 396 bp, Homo sapiens
tacatgattctaagaaacctggaccagaactatcaaaacttggtttcccagagaatatagcaaatgggctcattggccaatactatgacattggcttttgagaaaagaaaggctttattgcaaggctggccagcaaggagacaggagttgggctcaaatctgtctccccagtttggggcttagggcaagttttaattayacagacgcatttcttatgagtagcaggcagagagcctccaacttcttctgcctaggtaccagcagcttagacatgatgcaaacctgggaagcacatactgtatttggagaaagtgattgggaagaaatgtgagctgaggggaggggctcagtgcccctgagctacacttagtgatggcagaggaaggatgtcctccc

SEQ ID NO: 489 - DNA, 396 bp, Homo sapiens
tggggcttagggcaagttttaattacacagacgcatttcttatgagtagcaggcagagagcctccaacttcttctgcctaggtaccagcagcttagacatgatgcaaacctgggaagcacatactgtatttggagaaagtgattgggaagaaatgtgagctgaggggaggggctcagtgcccctgagctacacttagtratggcagaggaaggatgtcctcccgcaggaggctgttccacatctgctctggttgtagggggagctggcaggcattagcagcggcctctttcccccaagagaggcagcctcctccaagttttggcgacattatggccctgcaatcataagggtttgtgagcatagtgctaaggagggaaatggagctgctgttacta

SEQ ID NO: 490 - DNA, 396 bp, Homo sapiens
cctcctgagtagctaggactacaagcatgtgccaccacgcccagctaatttttgtatttttagtaaggacagggtttcaccatgttggccaggttggcctccaactcctgacctcaagtcatcctcctgcctcgacctcccaaagtgctgggattacaggcatgaaaccagcctagaaatacatactattatttattcytgttttacagataagcaaagtgagtcatggagaatttggttgaaagtcccaaggtcaggagtcgtgaagctgggattaaaacctaatcatctgactttagagagtagacacttgctccatgcatattgcctccaattcattcattcaagcactccctgctcaagaagttctttcttatgttgagctgaaatctgcag

SEQ ID NO: 491 - DNA, 396 bp, Homo sapiens
tcatctgactttagagagtagacacttgctccatgcatattgcctccaattcattcattcaagcactccctgctcaagaagttctttcttatgttgagctgaaatctgcagccctatgcgttttacccagcagtcctggtgctgttccctaaaatcacttagactgtgcctgctctttctgtgtttacagtgtcagctrtaatatccccctcttcggcctaacgtttctgaagtcccttgccactgggtctcctctcctcttcctgtgttctttctaagaacacctatgcagataggtgtcttctgtacagggaagctgttcctgagatccgggcatcgactctgttagaataatctacgtatgagttatttttttgagaactatgtgtcattgct

SEQ ID NO: 492 - DNA, 396 bp, Homo sapiens
atgttgagctgaaatctgcagccctatgcgttttacccagcagtcctggtgctgttccctaaaatcacttagactgtgcctgctctttctgtgtttacagtgtcagctgtaatatccccctcttcggcctaacgtttctgaagtcccttgccactgggtctcctctcctcttcctgtgttctttctaagaacacctatrcagataggtgtcttctgtacagggaagctgttcctgagatccgggcatcgactctgttagaataatctacgtatgagttatttttttgagaactatgtgtcattgctgactcatattaactctgtggttaactaaaatctcaagatctctttatgtttgttgagaaacttatttaacttctctggccctccgtttcc

SEQ ID NO: 493 - DNA, 396 bp, Homo sapiens
gtcctggtgctgttccctaaaatcacttagactgtgcctgctctttctgtgtttacagtgtcagctgtaatatccccctcttcggcctaacgtttctgaagtcccttgccactgggtctcctctcctcttcctgtgttctttctaagaacacctatgcagataggtgtcttctgtacagggaagctgttcctgagatcygggcatcgactctgttagaataatctacgtatgagttatttttttgagaactatgtgtcattgctgactcatattaactctgtggttaactaaaatctcaagatctctttatgtttgttgagaaacttatttaacttctctggccctccgtttccttcactgagcagtggagtgattgataacctccacctgtggtt

SEQ ID NO: 494 - DNA, 396 bp, Homo sapiens
cacctatgcagataggtgtcttctgtacagggaagctgttcctgagatccgggcatcgactctgttagaataatctacgtatgagttatttttttgagaactatgtgtcattgctgactcatattaactctgtggttaactaaaatctcaagatctctttatgtttgttgagaaacttatttaacttctctggccctcmgtttccttcactgagcagtggagtgattgataacctccacctgtggttgctgaaggtcttgcacaagatgatatagttaaagtagctagcagtgcccacgtacggcggatgcctcacaacggtttgcagccatctctctatctgtgtctttgtctctctctcacactggttttggcttactgttagcagctagccga

SEQ ID NO: 495 - DNA, 396 bp, Homo sapiens
tctgtggttaactaaaatctcaagatctctttatgtttgttgagaaacttatttaacttctctggccctccgtttccttcactgagcagtggagtgattgataacctccacctgtggttgctgaaggtcttgcacaagatgatatagttaaagtagctagcagtgcccacgtacggcggatgcctcacaacggtttgcmgccatctctctatctgtgtctttgtctctctctcacactggttttggcttactgttagcagctagccgagataagtgtgtttatggtctttgcatgtattgtttctgtagcatactggaggattacaagaggttggggagtgagggggcggtgaggagtagacaaaggcagccaactcttccaagtttagcttagaa

SEQ ID NO: 496 - DNA, 396 bp, Homo sapiens
ttgataacctccacctgtggttgctgaaggtcttgcacaagatgatatagttaaagtagctagcagtgcccacgtacggcggatgcctcacaacggtttgcagccatctctctatctgtgtctttgtctctctctcacactggttttggcttactgttagcagctagccgagataagtgtgtttatggtctttgcatgyattgtttctgtagcatactggaggattacaagaggttggggagtgagggggcggtgaggagtagacaaaggcagccaactcttccaagtttagcttagaaggaaggagcggtaaaccctagttgaatgttggactgaagcaggtttgtttttgttttgtttaaaggatagggaagatctgtgcgtgtttccaggata

SEQ ID NO: 497 - DNA, 396 bp, Homo sapiens
acttgaagtcagtggcatggacagggtcaagatcacagttagaggatgcagccttagagaaaaggaaggggctcggttctctgagcaaggagggaaagaagagaggcagatgcagagaagtacggcacatcgtgctgctggttgtagaaataacctctgacttttaataaagtcatccctcggtatccctgggggattrgttctatgacctccctcggatgccaaaattcgtggatgctcaagtccctgatataaaatggcatagtatttgcatttaacctacacacatcctccatatcctttttttttttttttttttttttttttttttttttgtgagatggagtcttgctctgtcgccctggctggagtacagtggctcgatcttggctcact

SEQ ID NO: 498 - DNA, 396 bp, Homo sapiens
aatacctgatagaatgtaaatgctatgtaaacagttgttatactgtattgttaaaagacagtaacaagaaaaaaaatctgtacatgttcagtccagacaaatggttttctgttttttttttttttttttaatatttttggtcagtggttggttgactccaggaatgcagaacccgcagatatagaaggttgattatgcrttcagaggcagggaataccatcttgggttccagaaagaaaatgatcagcattttctgtcatactctggtaaaaacagatcttttgaatggacaggtgtattaaaccctgtggagctggctgggcctggcggctcacgcctgtaatcccagcactttgggaggctgaggcaggtggatcacgaggtcaggagttcgag

SEQ ID NO: 499 - DNA, 396 bp, Homo sapiens
tgccccgcagagtttgaagtcccggctgcacctctccccagcagcaggttgactctggaaagttgcagcgttcttacctacagagtgggaacagtactacccattgcacagagtgggtgcaaagctctgtgacggaatacatggcaagtgcccaccacattgcctgggatgaggtgggcccttcctttacgtaagagarccctacagatacactcaaagtgggcacattcctacagaaggagtgttatttgtgtagaaaagaaaaacatgaaaggcttttattcctatacacaataaagcacccctttaatgtctttttgaggaggataatatgaaattgatgaaaaggaaccctgtggttggatccctgacaatcacatgtatcccttttttcac

SEQ ID NO: 500 - DNA, 396 bp, Homo sapiens
  misc_feature (227)..(326): n = A,T,C or G
tacagatacactcaaagtgggcacattcctacagaaggagtgttatttgtgtagaaaagaaaaacatgaaaggcttttattcctatacacaataaagcacccctttaatgtctttttgaggaggataatatgaaattgatgaaaaggaaccctgtggttggatccctgacaatcacatgtatcccttttttcactcttraaaaaggagtaaaggaataaaatagaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgtttcagtcactgtataataactagccagattttttgttgttgttgttttgtttttgtttttgttttt

SEQ ID NO: 501 - DNA, 396 bp, Homo sapiens
acattctgaaccacagacagttctttaccctgaacctttgcatattttgttctcttagcttagagcggcccctctccctccgtctgcttggctaatttctacttgttcttcagattttatcttagatgtcattccctcaaggaatccttctgtgactcaacatggaattaagttgcctcctttgaccctgaaagcaccrtgtactcaatctcatcttggcatgactcactttgctgtgtggaatgtctgctttccttgtttgtctattcctttagactgtaagatcctagaaagtgggggccgtgccttgctcatgactgtgtttctaacaccaaacacagtgttcagtagagagcagctgctgagtacgtttctgctaaatgacagttgatggag

SEQ ID NO: 502 - DNA, 396 bp, Homo sapiens
aatccttctgtgactcaacatggaattaagttgcctcctttgaccctgaaagcaccatgtactcaatctcatcttggcatgactcactttgctgtgtggaatgtctgctttccttgtttgtctattcctttagactgtaagatcctagaaagtgggggccgtgccttgctcatgactgtgtttctaacaccaaacacartgttcagtagagagcagctgctgagtacgtttctgctaaatgacagttgatggaggacatttagggttgcttggaggtcaagtcaaggaggcatttaacattctagtaaaacaaggaagtaacaggctcctgaacatgcccacaatgaaccagatgcaaaccttttcccttggcaggattctttgcccataaagtgg

SEQ ID NO: 503 - DNA, 396 bp, Homo sapiens
aaagcaccatgtactcaatctcatcttggcatgactcactttgctgtgtggaatgtctgctttccttgtttgtctattcctttagactgtaagatcctagaaagtgggggccgtgccttgctcatgactgtgtttctaacaccaaacacagtgttcagtagagagcagctgctgagtacgtttctgctaaatgacagtkgatggaggacatttagggttgcttggaggtcaagtcaaggaggcatttaacattctagtaaaacaaggaagtaacaggctcctgaacatgcccacaatgaaccagatgcaaaccttttcccttggcaggattctttgcccataaagtggagcacgaaagcaggacccagaatgggaggagcttccagaggaccggaa

SEQ ID NO: 504 - DNA, 396 bp, Homo sapiens
ttctgctaaatgacagttgatggaggacatttagggttgcttggaggtcaagtcaaggaggcatttaacattctagtaaaacaaggaagtaacaggctcctgaacatgcccacaatgaaccagatgcaaaccttttcccttggcaggattctttgcccataaagtggagcacgaaagcaggacccagaatgggaggagyttccagaggaccggaacacttgcctttgagcgggtctacactgccaagtgagtcctaaccctgatgttgctaataagtgggggcatgggcaggggggcctccttctaggagtgatgaccacccttaataccacatgtctgtctgagccaagtttctgagcgccagggaggtgaggaaggttggacttcaccagagag

SEQ ID NO: 505 - DNA, 396 bp, Homo sapiens
ggcatttaacattctagtaaaacaaggaagtaacaggctcctgaacatgcccacaatgaaccagatgcaaaccttttcccttggcaggattctttgcccataaagtggagcacgaaagcaggacccagaatgggaggagcttccagaggaccggaacacttgcctttgagcgggtctacactgccaagtgagtcctaamcctgatgttgctaataagtgggggcatgggcaggggggcctccttctaggagtgatgaccacccttaataccacatgtctgtctgagccaagtttctgagcgccagggaggtgaggaaggttggacttcaccagagaggctttgtggacaccctttatcatcttagtgagtgctagtgtcaaaacaaagggagtggg

SEQ ID NO: 506 - DNA, 396 bp, Homo sapiens
gctcctgaacatgcccacaatgaaccagatgcaaaccttttcccttggcaggattctttgcccataaagtggagcacgaaagcaggacccagaatgggaggagcttccagaggaccggaacacttgcctttgagcgggtctacactgccaagtgagtcctaaccctgatgttgctaataagtgggggcatgggcagggrggcctccttctaggagtgatgaccacccttaataccacatgtctgtctgagccaagtttctgagcgccagggaggtgaggaaggttggacttcaccagagaggctttgtggacaccctttatcatcttagtgagtgctagtgtcaaaacaaagggagtggggatatggggcacattggtggagggaggtgtgatctc

SEQ ID NO: 507 - DNA, 396 bp, Homo sapiens
ttgcccataaagtggagcacgaaagcaggacccagaatgggaggagcttccagaggaccggaacacttgcctttgagcgggtctacactgccaagtgagtcctaaccctgatgttgctaataagtgggggcatgggcaggggggcctccttctaggagtgatgaccacccttaataccacatgtctgtctgagccaagyttctgagcgccagggaggtgaggaaggttggacttcaccagagaggctttgtggacaccctttatcatcttagtgagtgctagtgtcaaaacaaagggagtggggatatggggcacattggtggagggaggtgtgatctctgcagcttcagaaagatctgaaagagtcatttggttagagaagttgacctatttcct

SEQ ID NO: 508 - DNA, 396 bp, Homo sapiens
aaacaaagggagtggggatatggggcacattggtggagggaggtgtgatctctgcagcttcagaaagatctgaaagagtcatttggttagagaagttgacctatttcctgtggggttagaccagggttgctactgtgaacaccagccatgactcaccagtcaccttcagaagccacaggcaggacatgctgacgacagycttcaactcacccaccccttgctcccctgcgggtggaagtctggaggtgacaccactgcattttctaacacgggggctccttgagcaactagaacaagaacagaaagaatggggacattagcaggtgctttccccctctctcattcttttctttgaataaaaaggttgtttgaaaacacctgagcggctcctaaaga

SEQ ID NO: 509 - DNA, 396 bp, Homo sapiens
ctcctctcttctttatgcagagtgtatttcaaggctcagccagtggcaggcatgctggggactatggactacggactaggggcctgtcacagaggaaggcctcatgctagagagctaagggaggagctggccttcagttccatcccaggagcaactttgatgttcccagagatccttccaaagggggagtcatggtcamccaagaaaaatgtattcagaatgccaagaatggtgcaaactcaggacaaagattcacactgcagggttggagtccctgggcttgctgctggcaccatgggagggagggtccccttcaggggtaccgttggtttcctgtgaattaaactggcttcaagggatctcgactgaacaggcctatatcacactcactgatat

SEQ ID NO: 510 - DNA, 396 bp, Homo sapiens
tctcctcatctaggtatttttaattgtttcagtgaggtgtaggcatgaggggattggagggggcatctcctccattgcagtttttcattggctgctttgctccctcagctccgaaatcgctgggccactctcgaacgcattagtacggtagtcacaggttgattgcctggccccttgccctctgtgggcattttccctytcagacagcccctgagtactcacagtgctgctacagtgggccacctagatctccctctttctccatgctcccacgtgctctgggctccactcccttctcccaagcacttctgtccagggctattccagcagtctgacctcaaggaaatcctttgctaaactgattatagagaggtttctattttaacatttaggtct

SEQ ID NO: 511 - DNA, 396 bp, Homo sapiens
atctaggtatttttaattgtttcagtgaggtgtaggcatgaggggattggagggggcatctcctccattgcagtttttcattggctgctttgctccctcagctccgaaatcgctgggccactctcgaacgcattagtacggtagtcacaggttgattgcctggccccttgccctctgtgggcattttccctttcagacwgcccctgagtactcacagtgctgctacagtgggccacctagatctccctctttctccatgctcccacgtgctctgggctccactcccttctcccaagcacttctgtccagggctattccagcagtctgacctcaaggaaatcctttgctaaactgattatagagaggtttctattttaacatttaggtcttccatgt

SEQ ID NO: 512 - DNA, 396 bp, Homo sapiens
aggtgtaggcatgaggggattggagggggcatctcctccattgcagtttttcattggctgctttgctccctcagctccgaaatcgctgggccactctcgaacgcattagtacggtagtcacaggttgattgcctggccccttgccctctgtgggcattttccctttcagacagcccctgagtactcacagtgctgctayagtgggccacctagatctccctctttctccatgctcccacgtgctctgggctccactcccttctcccaagcacttctgtccagggctattccagcagtctgacctcaaggaaatcctttgctaaactgattatagagaggtttctattttaacatttaggtcttccatgtattaattctcagaatcaatttaagatg

SEQ ID NO: 513 - DNA, 396 bp, Homo sapiens
cctttcagacagcccctgagtactcacagtgctgctacagtgggccacctagatctccctctttctccatgctcccacgtgctctgggctccactcccttctcccaagcacttctgtccagggctattccagcagtctgacctcaaggaaatcctttgctaaactgattatagagaggtttctattttaacatttaggycttccatgtattaattctcagaatcaatttaagatgtttaaaggtgtgatttaagacattttaaaaccatttggaggagagtacagaaattatgtcacttgctgtcagcctctttgcaccatctgcagagaaagatactagagtcccgccttggacacatccacatgcaagaggtgcaaagaaggtgtctttgatga

SEQ ID NO: 514 - DNA, 396 bp, Homo sapiens
ttctcagaatcaatttaagatgtttaaaggtgtgatttaagacattttaaaaccatttggaggagagtacagaaattatgtcacttgctgtcagcctctttgcaccatctgcagagaaagatactagagtcccgccttggacacatccacatgcaagaggtgcaaagaaggtgtctttgatgaggcaaggtcaaaactyctccccagacgaaatccaaagaaagcattcctactatgctatatcagtttggaaagaaaaacttctgccaggtgactgcattctcactggtcacattgtgttcctatggactcctcagctcaaccaatttggagaagttatggtgcaatttcaccatatctggttagaagttaagtttccaatttgctggcaatgaa

SEQ ID NO: 515 - DNA, 396 bp, Homo sapiens
aagaaggtgtctttgatgaggcaaggtcaaaacttctccccagacgaaatccaaagaaagcattcctactatgctatatcagtttggaaagaaaaacttctgccaggtgactgcattctcactggtcacattgtgttcctatggactcctcagctcaaccaatttggagaagttatggtgcaatttcaccatatctggytagaagttaagtttccaatttgctggcaatgaagaagaaatggagcaggccaggctgtgtagtttctgccacgtgcccccgggagtgaacagctctgtttgtaagaagccatggtgcttagacctgggctcgctagttgccagcctccaaattgcagaagtgccctttggttggtggctatgctgtgtcacttggga

SEQ ID NO: 516 - DNA, 396 bp, Homo sapiens
gcaacatatctgtgtgcctgtctgggttgtaaaaagggtcaaagatcaatgcagcaggcagctacatgctggcaaaagccagaggcagctggtctgtttgcctgtgccaggaaaccactgggaatggggttgtgtgttattctaggagaaagtcgtcccagcagcagcttctccaggggcatccaagagcactgaaaarggttgcaagatgacccatgaggctgcaggaagaaaagaacatgcatttaatcttgctatctgaaaagtaagacatgaagctttcctcatttttaatatacacatggacagtagtatgtgtatatagtttatatgcaaatatacttgttataaggttgcatgctcaaaatttttggttcatggggtgtgggatcataa

SEQ ID NO: 517 - DNA, 396 bp, Homo sapiens
cagctacatgctggcaaaagccagaggcagctggtctgtttgcctgtgccaggaaaccactgggaatggggttgtgtgttattctaggagaaagtcgtcccagcagcagcttctccaggggcatccaagagcactgaaaagggttgcaagatgacccatgaggctgcaggaagaaaagaacatgcatttaatcttgctrtctgaaaagtaagacatgaagctttcctcatttttaatatacacatggacagtagtatgtgtatatagtttatatgcaaatatacttgttataaggttgcatgctcaaaatttttggttcatggggtgtgggatcataaatgtttagggaccatggctatcaaggaaaaacagcatgaaggataaatgatactggtg

SEQ ID NO: 518 - DNA, 396 bp, Homo sapiens
ctatctgaaaagtaagacatgaagctttcctcatttttaatatacacatggacagtagtatgtgtatatagtttatatgcaaatatacttgttataaggttgcatgctcaaaatttttggttcatggggtgtgggatcataaatgtttagggaccatggctatcaaggaaaaacagcatgaaggataaatgatactggyggattaaaaagacagatgcatgtatttttagcataaaacacaactgctgactgatacagatagctcaagattctggggcagctgctgaacagatacactagccagtgtggctcatcggctcagacttggccttaattaatgggctgtccctccacccatctcccatgagggcagagctgagccagggtttgagagct

SEQ ID NO: 519 - DNA, 396 bp, Homo sapiens
agtttatatgcaaatatacttgttataaggttgcatgctcaaaatttttggttcatggggtgtgggatcataaatgtttagggaccatggctatcaaggaaaaacagcatgaaggataaatgatactggtggattaaaaagacagatgcatgtatttttagcataaaacacaactgctgactgatacagatagctcaasattctggggcagctgctgaacagatacactagccagtgtggctcatcggctcagacttggccttaattaatgggctgtccctccacccatctcccatgagggcagagctgagccagggtttgagagctaaaaggaattggacctggactctgttcacgtgtatattttaattctaattaattcattcttttgaaaga

SEQ ID NO: 520 - DNA, 394 bp, Homo sapiens
gtatttttagcataaaacacaactgctgactgatacagatagctcaagattctggggcagctgctgaacagatacactagccagtgtggctcatcggctcagacttggccttaattaatgggctgtccctccacccatctcccatgagggcagagctgagccagggtttgagagctaaaaggaattggacctggactcdgttcacgtgtatattttaattctaattaattcattcttttgaaagacagagtcacactctgttgcctaggctggagtgcagtggcacgatcttggctcactgcaacctcggcctcccaggttcaagttattctcctgcttcagcctcctgagtagctgggattataggcacatgcccccatgcctgactaatttt

SEQ ID NO: 521 - DNA, 396 bp, Homo sapiens
gctaaaaggaattggacctggactctgttcacgtgtatattttaattctaattaattcattcttttgaaagacagagtcacactctgttgcctaggctggagtgcagtggcacgatcttggctcactgcaacctcggcctcccaggttcaagttattctcctgcttcagcctcctgagtagctgggattataggcacaygcccccatgcctgactaatttttgtatttttagtagagacggggtttcaccatgtcaggctggtcttgaactcctgacctcaggttatccacccgccttggcccctcaaagtgttggaattacaggtgtgagccaccgtgcctggcctgttcacatgtataaaacacagtttaatgtcctattcccagccaatgagc

SEQ ID NO: 522 - DNA, 396 bp, Homo sapiens
tcaggttatccacccgccttggcccctcaaagtgttggaattacaggtgtgagccaccgtgcctggcctgttcacatgtataaaacacagtttaatgtcctattcccagccaatgagcatggctagagcagccttggtcaaagtttggtttttggagaaaaatccttgttagctgacctaagattcctctttgtgagtktaagtaagcacaggttgcagagaggagaagggtctctggagaggtgtaattttctaaatggattacaagttcatggacttttaacaggtgttacaggggataacaagttctttatagacagacttttgaggacgtttaagggtattctgattcttggttttctaagaggggaatgtattatttaactacagacaccc

SEQ ID NO: 523 - DNA, 396 bp, Homo sapiens
aaaatccagaataataataatttgtcaataggaaagacatttccactgggggttaagaaggaagacattggaacaatgatagccaccacttattgaatgcttactgtgagccaggtggcacttcaccttgtttcattctcacaacagtctagggaagtaattactaatgtctccatccacctcttgtagatgagcaaaytgaggctcattgaggctaggaaatgcacccacactcacatagcccataagaggcagccatggcattgggcccagaccatgtgaacttcaaagactacacgagcagccactgggcagctgtcatggctaaagccacttgaattcagcccagcagcaaccccctctccaggaggggcacataagcttgcagctttgggt

SEQ ID NO: 524 - DNA, 396 bp, Homo sapiens
ataataataatttgtcaataggaaagacatttccactgggggttaagaaggaagacattggaacaatgatagccaccacttattgaatgcttactgtgagccaggtggcacttcaccttgtttcattctcacaacagtctagggaagtaattactaatgtctccatccacctcttgtagatgagcaaactgaggctcaytgaggctaggaaatgcacccacactcacatagcccataagaggcagccatggcattgggcccagaccatgtgaacttcaaagactacacgagcagccactgggcagctgtcatggctaaagccacttgaattcagcccagcagcaaccccctctccaggaggggcacataagcttgcagctttgggtagaagctgca

SEQ ID NO: 525 - DNA, 396 bp, Homo sapiens
gcacttgaagtcctggatggcgagagggactggcttgagccagagccaggaacaaggctctgagaatattctggaaatccacaggaggaacccattttcttacagctgggagaatttcattcaactccaggctgaccatgttttattaggaacgaaggtgacttgaactaatagtcaggaatggttgaatacggacccratgtcaaatcactaggcagttcacatttctaatgagcaaatcccttagacaattaagaatttttttccttttgcataacccagacaaaatcgctacttaaaaacaaaccaaagacccgaaacatgagaaagagaaggaagcaggggaaatctttggtactaataagtttttaaacaataagagcaccagatatttta

SEQ ID NO: 526 - DNA, 396 bp, Homo sapiens
atgagcaaatcccttagacaattaagaatttttttccttttgcataacccagacaaaatcgctacttaaaaacaaaccaaagacccgaaacatgagaaagagaaggaagcaggggaaatctttggtactaataagtttttaaacaataagagcaccagatattttaccccatcagacacagaatgttattcgaataacsaaaaaaggaattttttctctaagtttcttgaactggaaaatgaatcatattttctcagtcctgaggctgcaattttgtgcctctagtaacatataagaatagatgtgatgccagtgcccagtagctgctgcaattgttacttggggacctgtttattcactaagcacttcaccccagtgataaatttgtaggggcct

SEQ ID NO: 527 - DNA, 396 bp, Homo sapiens
ccgtgtccattagatcagtggaaattctgggattcagagcactttgcaaggtcagcaggggtctgctctttctgtcctgttcctggtttttggttgtgcctggattccagggtaggtttctcatctgttaccttcatagacttctccagaaaaggatcttttgaccatcagaggaccacgaagattccattggtgaggygcagataacctgatctctctgggttctctgcagggcacagatgaagggctggccattcccaagttctcagtggtaccactgaggcatgagaccctaatggtttgcatgagcagtttgaaaattgcatctttgtttttacctatataatcacatgaaacccgtggttctcaaacgtcagcaggcatcagcatcacatg

SEQ ID NO: 528 - DNA, 396 bp, Homo sapiens
tcagtggtaccactgaggcatgagaccctaatggtttgcatgagcagtttgaaaattgcatctttgtttttacctatataatcacatgaaacccgtggttctcaaacgtcagcaggcatcagcatcacatggagggcttgttaaaacagatttctgggccccaacacagagttttaaattctgaaggcctgaggtgggygtgaacatttgcatttctaacatgttctcgatgctgctgccgcctctggtcccgagagcatgcctggagaactgccaccttcgaccatggactgtgagaattcacatggacctcagaattataatcagtctctcagttttacagataaggaaactaaatccagagagattgttttgccaatggtgaacagctggtta

SEQ ID NO: 529 - DNA, 396 bp, Homo sapiens
atggtttgcatgagcagtttgaaaattgcatctttgtttttacctatataatcacatgaaacccgtggttctcaaacgtcagcaggcatcagcatcacatggagggcttgttaaaacagatttctgggccccaacacagagttttaaattctgaaggcctgaggtgggtgtgaacatttgcatttctaacatgttctcratgctgctgccgcctctggtcccgagagcatgcctggagaactgccaccttcgaccatggactgtgagaattcacatggacctcagaattataatcagtctctcagttttacagataaggaaactaaatccagagagattgttttgccaatggtgaacagctggttaaagtcaggatggagactttaatcctagtca

SEQ ID NO: 530 - DNA, 396 bp, Homo sapiens
gagcagtttgaaaattgcatctttgtttttacctatataatcacatgaaacccgtggttctcaaacgtcagcaggcatcagcatcacatggagggcttgttaaaacagatttctgggccccaacacagagttttaaattctgaaggcctgaggtgggtgtgaacatttgcatttctaacatgttctcgatgctgctgcygcctctggtcccgagagcatgcctggagaactgccaccttcgaccatggactgtgagaattcacatggacctcagaattataatcagtctctcagttttacagataaggaaactaaatccagagagattgttttgccaatggtgaacagctggttaaagtcaggatggagactttaatcctagtcaagtgacctttc

SEQ ID NO: 531 - DNA, 396 bp, Homo sapiens
agtttgaaaattgcatctttgtttttacctatataatcacatgaaacccgtggttctcaaacgtcagcaggcatcagcatcacatggagggcttgttaaaacagatttctgggccccaacacagagttttaaattctgaaggcctgaggtgggtgtgaacatttgcatttctaacatgttctcgatgctgctgccgcckctggtcccgagagcatgcctggagaactgccaccttcgaccatggactgtgagaattcacatggacctcagaattataatcagtctctcagttttacagataaggaaactaaatccagagagattgttttgccaatggtgaacagctggttaaagtcaggatggagactttaatcctagtcaagtgacctttcctct

SEQ ID NO: 532 - DNA, 396 bp, Homo sapiens
catctttgtttttacctatataatcacatgaaacccgtggttctcaaacgtcagcaggcatcagcatcacatggagggcttgttaaaacagatttctgggccccaacacagagttttaaattctgaaggcctgaggtgggtgtgaacatttgcatttctaacatgttctcgatgctgctgccgcctctggtcccgagakcatgcctggagaactgccaccttcgaccatggactgtgagaattcacatggacctcagaattataatcagtctctcagttttacagataaggaaactaaatccagagagattgttttgccaatggtgaacagctggttaaagtcaggatggagactttaatcctagtcaagtgacctttcctctgtatttatttccc

SEQ ID NO: 533 - DNA, 396 bp, Homo sapiens
atttctgacatcctgaaccatagtaaaagggtgttttttgtttttttgagacagagtcttgctctgttgcctgggctggagtgcagtggtgtgatcttggctcgctgcaacctccgcctcccaggttcaagtgattctcctgcctcagcctcctgagtagctgggattacaggtgcttgccaccacacctggctatttkttgtgtttttagtagagacagggtttcaccatgttggccaggctggtcttgaactcctgaccttgtgatctgcctgcctcagcctcccaaattgctgggattacaaggcgtgttgttttaagccactcagtttgtggccacttgttacagcagcaagaggaaactcatacagttatcatgtgaactcacaggaatat

SEQ ID NO: 534 - DNA, 396 bp, Homo sapiens
gatctgcctgcctcagcctcccaaattgctgggattacaaggcgtgttgttttaagccactcagtttgtggccacttgttacagcagcaagaggaaactcatacagttatcatgtgaactcacaggaatatggtgagttaaaaagagaggaagggtgcaaaacatccacggtagagtgagaactctccagggagtgagractgtgcccagcatacagtgatcaccctcttagtaagctaagtttctgagcaccagcttttttgagttgactttgttgtctttaacatttgaagatcacccttctttgctcagcctggcttgcagacctgggctgatttgtggatctgatagaaaagtttccttagttgggctcttctccccgaccacccccatgcc

SEQ ID NO: 535 - DNA, 396 bp, Homo sapiens
tgcctcagcctcccaaattgctgggattacaaggcgtgttgttttaagccactcagtttgtggccacttgttacagcagcaagaggaaactcatacagttatcatgtgaactcacaggaatatggtgagttaaaaagagaggaagggtgcaaaacatccacggtagagtgagaactctccagggagtgaggactgtgcmcagcatacagtgatcaccctcttagtaagctaagtttctgagcaccagcttttttgagttgactttgttgtctttaacatttgaagatcacccttctttgctcagcctggcttgcagacctgggctgatttgtggatctgatagaaaagtttccttagttgggctcttctccccgaccacccccatgccagtgtggc

SEQ ID NO: 536 - DNA, 396 bp, Homo sapiens
gctactttgcagccaaggtaactcagacttccctttgttcattctccttctataaagtgcatctcaaggaggttcaaagggcaggctttttgttgaaaggactttgcctgacctctggctcccatctgtgaagccctggagaggtgagagccctcgggaggccgtgtttcaggcatgctctgcacccgtgcagagcgcrtgtgataatgcattgctaatgcttgctccctggtggctggctgagagctgctgtgctgacaagggtggtttaaggctaaatgtgactcagaatccttaagcagtgttagttcagatacaagggcattataaatgagagtgcctgagggatctattttgggaccgctgtcacttggctcttctgctaataagcttcca

SEQ ID NO: 537 - DNA, 396 bp, Homo sapiens
acagttatcagcagcccacaggcttgacttgagcaagttggaaagacaaatcaacttccagagttgatttaacattgagtggaaatcagtcatacttttggtcccctttcggggccacgcctggcactgtgcctggtggcagatcggcatgaactggccagcttctgtggccctggagggcacaggcagaaaggccacrctcagtcccatgatgaactgtttaagacttattgttgtctccccgctctgtaaagtagatagagtggattttatgtcccttattacctttcaggatactttgactcagggagataaagtaacttgggtacagctactcagctggtgaagaacacaggcagaatgagtgcctgggtcttttgacttaaaattctggat

SEQ ID NO: 538 - DNA, 396 bp, Homo sapiens
ctgtgcctggtggcagatcggcatgaactggccagcttctgtggccctggagggcacaggcagaaaggccacactcagtcccatgatgaactgtttaagacttattgttgtctccccgctctgtaaagtagatagagtggattttatgtcccttattacctttcaggatactttgactcagggagataaagtaacttgsgtacagctactcagctggtgaagaacacaggcagaatgagtgcctgggtcttttgacttaaaattctggatttttcacaaagatcctcttactttattcatttacataataaatatatattgaagagctactctgtgccaagccctgtgcctagatatacagtgataaataaagagtagcttctagaggtcacctgg

SEQ ID NO: 539 - DNA, 396 bp, Homo sapiens
aagttcagtgatagagagcagaggtgaggcggcagcagaaaccacttaagggacaccacgtggcactccttctgtgctgagaaggctgtcagtaagctcaccatttatttcctattttctctcctgagttaaataggaaacatgtctcgcattacttgaaaaatcaagtcaaactatgctcttactaggagttatggtyctttttatgtcttagatgatgcttgatctagatgaatgcggacttgctgtagctagataaatacaatgggagtttgaaggtgtttcgtagccctggaaataggtatttcctgtcaaaacaagctttgtcattgccagcagacaaaagcatcagtaaccttggttgataatcgtcatttcttaggaataaagtagact

SEQ ID NO: 540 - DNA, 396 bp, Homo sapiens
gtatttcctgtcaaaacaagctttgtcattgccagcagacaaaagcatcagtaaccttggttgataatcgtcatttcttaggaataaagtagactgtagaattttttttagcagaaaggaaacccaaagataattctagtgcaaatccctcactttatagagcagaagctcaagtcccagaggaacaagtggcttgaaygaacatcagaattttaggggctggatttgtaccctcctggtgccagcagcccacttccctgcaggaggcactcaccttccttgcacaggggtatgagtgtggccattttccacccataatctctgttagctcatgttcaattgggttcccattgaaagaaaaatggaccagtaagttggagcagaatcattcagatg

SEQ ID NO: 541 - DNA, 396 bp, Homo sapiens
agctttgtcattgccagcagacaaaagcatcagtaaccttggttgataatcgtcatttcttaggaataaagtagactgtagaattttttttagcagaaaggaaacccaaagataattctagtgcaaatccctcactttatagagcagaagctcaagtcccagaggaacaagtggcttgaacgaacatcagaattttagkggctggatttgtaccctcctggtgccagcagcccacttccctgcaggaggcactcaccttccttgcacaggggtatgagtgtggccattttccacccataatctctgttagctcatgttcaattgggttcccattgaaagaaaaatggaccagtaagttggagcagaatcattcagatggtataacataaggaaaaa

SEQ ID NO: 542 - DNA, 396 bp, Homo sapiens
tgtttaaattgcttttatatctgtagctctagataacactagttccagcttagttaactcccagctccaagccttcaggacttcatagagttattggggtgctgctcttggcagtttcccaaaaagctagaatgcagagggaatctccttcccaaaaagctagaatgcagagggaatctccttcccaaaaggctagaaygcagagggaatctccttcccaaaaagctagaatgcagagggaatctccttcccaaaaggctagaacgcagagggaatctccttcccaaaaggctagaacgcagagggaatctccttcccaaaaggctagaatgcagagggaatgtccttctcttctaaatggtagctgttagttcaagaaaggttaaacattgtgct

SEQ ID NO: 543 - DNA, 396 bp, Homo sapiens
gctgcgtttgctggactgatgtacttgtttgtgaggcaaaagtactttgtcggttacctaggagagagaacgcagaggtaggtaactgggactactaaagaactgtggagcgattcctgatttttgagcaggaagagtgacaattcaaaacagtatttgactagattcacggctccgtagcatccccttgggtgggagsgggaaggctgactaggacctctgattcttctttccctgagctttgaaggctctgaaaatacagctggggggacttgcccagttttcttattaagcaattcctccgcatggtgctggctttcaaagggtgcttcagtgctgtttgctgcacgtgccttgcagccccacaccctgcactcccgccctgcagagtctggc

SEQ ID NO: 544 - DNA, 396 bp, Homo sapiens
gaggcaaaagtactttgtcggttacctaggagagagaacgcagaggtaggtaactgggactactaaagaactgtggagcgattcctgatttttgagcaggaagagtgacaattcaaaacagtatttgactagattcacggctccgtagcatccccttgggtgggagggggaaggctgactaggacctctgattcttctytccctgagctttgaaggctctgaaaatacagctggggggacttgcccagttttcttattaagcaattcctccgcatggtgctggctttcaaagggtgcttcagtgctgtttgctgcacgtgccttgcagccccacaccctgcactcccgccctgcagagtctggcgctggaatgacattttaggtctgggttcccag

SEQ ID NO: 545 - DNA, 396 bp, Homo sapiens
tatctttcagggaccagaagaaagaatgttgggaaaataagatgcagtaagatgcagacatgacagcagggtgcagcggctcacgcctataatcccagcactttgggaggctgaggtgggtggatcacctgaggtcaggagtttgagaccagcctggccaacatggtgaaaccccgtctctactaaaaaatatacaaarcattagccaggcatggtggtgggcgcctgtaatcccagctactccataggctgaggctggagaatcgcttgaacccaggaggcagaggttgcagtgagccgagattgcgccactgcactccagcctgggcaacaaaagcaaaactccatctcaaaaaaaaaaaaaaaaaaaaaaaaaaagatgcagacacgagactg

SEQ ID NO: 546 - DNA, 396 bp, Homo sapiens
tgggcgcctgtaatcccagctactccataggctgaggctggagaatcgcttgaacccaggaggcagaggttgcagtgagccgagattgcgccactgcactccagcctgggcaacaaaagcaaaactccatctcaaaaaaaaaaaaaaaaaaaaaaaaaaagatgcagacacgagactgtgaaactgactagcatcaccwttgcattgtttatagatgttgccagacagaaagccccaaagcagcacagtaccttcctgacatctggactaggaaatctagattttagtaaaatacatgctaatacttacagaagaaatgtcggcgttagagtatgccgtcagttccttagagattgcaattcctaatgcactagtatggtttcaggtgccaggaac

SEQ ID NO: 547 - DNA, 396 bp, Homo sapiens
actccatctcaaaaaaaaaaaaaaaaaaaaaaaaaaagatgcagacacgagactgtgaaactgactagcatcaccattgcattgtttatagatgttgccagacagaaagccccaaagcagcacagtaccttcctgacatctggactaggaaatctagattttagtaaaatacatgctaatacttacagaagaaatgtcrgcgttagagtatgccgtcagttccttagagattgcaattcctaatgcactagtatggtttcaggtgccaggaacacgttctgtgaggctgctgccccaggtgctgaccccagccttccacaccattttccttccttgtgttcacagccgctctgtcttttacaatagcacccctctctagtggctaatgggctctat

SEQ ID NO: 548 - DNA, 396 bp, Homo sapiens
aaaaaaaaaaaaaaaaaaaaaagatgcagacacgagactgtgaaactgactagcatcaccattgcattgtttatagatgttgccagacagaaagccccaaagcagcacagtaccttcctgacatctggactaggaaatctagattttagtaaaatacatgctaatacttacagaagaaatgtcggcgttagagtatgcygtcagttccttagagattgcaattcctaatgcactagtatggtttcaggtgccaggaacacgttctgtgaggctgctgccccaggtgctgaccccagccttccacaccattttccttccttgtgttcacagccgctctgtcttttacaatagcacccctctctagtggctaatgggctctatgattagatagcatcc

SEQ ID NO: 549 - DNA, 396 bp, Homo sapiens
tttcaggtgccaggaacacgttctgtgaggctgctgccccaggtgctgaccccagccttccacaccattttccttccttgtgttcacagccgctctgtcttttacaatagcacccctctctagtggctaatgggctctatgattagatagcatccttcagtagtgataaaggcagtgacatcctagggaggtcagcggktgaaagcgctatatctggaaaacctgagagcctgtgaagctcaaggacttgacggggttagaccgtgagccgggctgcagctggaaaaagaatgactgttctttcagcagatccttccctgtgccatctctttcttcattcctctctagtggcattcttatttatcctctaaaaccacaattccattatctctccta

SEQ ID NO: 550 - DNA, 396 bp, Homo sapiens
gagggtcttctcttttgcctggctccctatgcagccctatcttaccccctgcaaagtcccagggatgtggctcagtcactgctcctctcttcatctgtcaccacttgcttgagatcctacagctgctttaattccgagaccatctgcagaacatgacaaaatttgtccacctacccacatgtccttttaactttaaagrctttactaactgattcctattagggaatgaacagaggtggcaaaaataaacaataggagattgatttacaagaaatctttaaaatagtagatttcttcggacctcattgaaatataaatggcctgccttcttgtgtccctccctggtctccctctttaggtgataagaagaagatcctgccagccccataacccgcc

SEQ ID NO: 551 - DNA, 396 bp, Homo sapiens
ttaaaatagtagatttcttcggacctcattgaaatataaatggcctgccttcttgtgtccctccctggtctccctctttaggtgataagaagaagatcctgccagccccataacccgccatctgcgcgggttctagacccccttctcctcccctctggccgtggtaggcattactgatgaatcatggtgctctttcttmcagagaccaaacctggcctcggaatccttcttaacacagatactgcttaacacaaccactctgagcagctgtcataagtagaagtaatagatactagaagaaatgtctaagcctaatctagaccaaaatacggcctgatatagatgcaagccagaggggctttatggttaaatgcaaggagattttcaaccctgccg

SEQ ID NO: 552 - DNA, 396 bp, Homo sapiens
ctggtctccctctttaggtgataagaagaagatcctgccagccccataacccgccatctgcgcgggttctagacccccttctcctcccctctggccgtggtaggcattactgatgaatcatggtgctctttcttccagagaccaaacctggcctcggaatccttcttaacacagatactgcttaacacaaccactctgrgcagctgtcataagtagaagtaatagatactagaagaaatgtctaagcctaatctagaccaaaatacggcctgatatagatgcaagccagaggggctttatggttaaatgcaaggagattttcaaccctgccgtctagaagctacttgctgagatcttcttcagttgggcccatctcctccccaggcctctcttctg

SEQ ID NO: 553 - DNA, 396 bp, Homo sapiens
ccataacccgccatctgcgcgggttctagacccccttctcctcccctctggccgtggtaggcattactgatgaatcatggtgctctttcttccagagaccaaacctggcctcggaatccttcttaacacagatactgcttaacacaaccactctgagcagctgtcataagtagaagtaatagatactagaagaaatgtmtaagcctaatctagaccaaaatacggcctgatatagatgcaagccagaggggctttatggttaaatgcaaggagattttcaaccctgccgtctagaagctacttgctgagatcttcttcagttgggcccatctcctccccaggcctctcttctgttcctgggctatgtcacacttggactctgcagacacctaatgc

SEQ ID NO: 554 - DNA, 396 bp, Homo sapiens
tggtaggcattactgatgaatcatggtgctctttcttccagagaccaaacctggcctcggaatccttcttaacacagatactgcttaacacaaccactctgagcagctgtcataagtagaagtaatagatactagaagaaatgtctaagcctaatctagaccaaaatacggcctgatatagatgcaagccagaggggckttatggttaaatgcaaggagattttcaaccctgccgtctagaagctacttgctgagatcttcttcagttgggcccatctcctccccaggcctctcttctgttcctgggctatgtcacacttggactctgcagacacctaatgctcttgggacctgctttagttcttgacctcaccaaccgaggaggaattgctagat

SEQ ID NO: 555 - DNA, 396 bp, Homo sapiens
cagagaccaaacctggcctcggaatccttcttaacacagatactgcttaacacaaccactctgagcagctgtcataagtagaagtaatagatactagaagaaatgtctaagcctaatctagaccaaaatacggcctgatatagatgcaagccagaggggctttatggttaaatgcaaggagattttcaaccctgccgtytagaagctacttgctgagatcttcttcagttgggcccatctcctccccaggcctctcttctgttcctgggctatgtcacacttggactctgcagacacctaatgctcttgggacctgctttagttcttgacctcaccaaccgaggaggaattgctagatgagatccttcccccggaatttctctcttgaaccccaga

SEQ ID NO: 556 - DNA, 396 bp, Homo sapiens
gggctttatggttaaatgcaaggagattttcaaccctgccgtctagaagctacttgctgagatcttcttcagttgggcccatctcctccccaggcctctcttctgttcctgggctatgtcacacttggactctgcagacacctaatgctcttgggacctgctttagttcttgacctcaccaaccgaggaggaattgctmgatgagatccttcccccggaatttctctcttgaaccccagatggtccgttgcccctttccagaagttgctccagccctgtccgcttaggaagttcagtgtcatccttgatccagtgggtagggaagacattccataatgaatgccccagtctgagcttcttccttcaggcttcaggctgccctgcgaggattttgca

SEQ ID NO: 557 - DNA, 396 bp, Homo sapiens
gtagctgagactacaggtgtgcactaccacacccagctaattttttgtatttttagtagagatagggtttagctatgttggccaggctggtctcgaactgctgaactcaagcaatctgccatccccggcctcccaaagtactgggagtataggcataagccacccatgatgcccagcctgaatcttggtttcttccccrttcatttaagctattacctgggcctgaactcaatggcacctggcaccaactggcaactgactcttggtcttttattacctaccttccctagcaggcactgggttgctccctcttcctatcccatggagtcctgtcctctgttggggctcctactgatcctcttggcaatatgaagttctcagctcaatggtgggtgg

SEQ ID NO: 558 - DNA, 396 bp, Homo sapiens
cccggcctcccaaagtactgggagtataggcataagccacccatgatgcccagcctgaatcttggtttcttccccattcatttaagctattacctgggcctgaactcaatggcacctggcaccaactggcaactgactcttggtcttttattacctaccttccctagcaggcactgggttgctccctcttcctatcccrtggagtcctgtcctctgttggggctcctactgatcctcttggcaatatgaagttctcagctcaatggtgggtgggcaatgactgccaactcttgaggccaatgaactcaggttaccccactcctcctcctcctgagttgctcactcactcctcattcactcaacattgattcagtagatatttgctacctgctctgt

SEQ ID NO: 559 - DNA, 396 bp, Homo sapiens
ccggcctcccaaagtactgggagtataggcataagccacccatgatgcccagcctgaatcttggtttcttccccattcatttaagctattacctgggcctgaactcaatggcacctggcaccaactggcaactgactcttggtcttttattacctaccttccctagcaggcactgggttgctccctcttcctatcccayggagtcctgtcctctgttggggctcctactgatcctcttggcaatatgaagttctcagctcaatggtgggtgggcaatgactgccaactcttgaggccaatgaactcaggttaccccactcctcctcctcctgagttgctcactcactcctcattcactcaacattgattcagtagatatttgctacctgctctgtg

SEQ ID NO: 560 - DNA, 396 bp, Homo sapiens
ggcataagccacccatgatgcccagcctgaatcttggtttcttccccattcatttaagctattacctgggcctgaactcaatggcacctggcaccaactggcaactgactcttggtcttttattacctaccttccctagcaggcactgggttgctccctcttcctatcccatggagtcctgtcctctgttggggctccyactgatcctcttggcaatatgaagttctcagctcaatggtgggtgggcaatgactgccaactcttgaggccaatgaactcaggttaccccactcctcctcctcctgagttgctcactcactcctcattcactcaacattgattcagtagatatttgctacctgctctgtgccaggtaccaggtcagttgctgaagga

SEQ ID NO: 561 - DNA, 396 bp, Homo sapiens
cctggcaccaactggcaactgactcttggtcttttattacctaccttccctagcaggcactgggttgctccctcttcctatcccatggagtcctgtcctctgttggggctcctactgatcctcttggcaatatgaagttctcagctcaatggtgggtgggcaatgactgccaactcttgaggccaatgaactcaggttwccccactcctcctcctcctgagttgctcactcactcctcattcactcaacattgattcagtagatatttgctacctgctctgtgccaggtaccaggtcagttgctgaaggagtaacagtgaacatgacggagtctttgtccccaaggagacccaaggtgtctcctagagccaggggcacattgcaagaccaaatata

SEQ ID NO: 562 - DNA, 396 bp, Homo sapiens
ctggcaactgactcttggtcttttattacctaccttccctagcaggcactgggttgctccctcttcctatcccatggagtcctgtcctctgttggggctcctactgatcctcttggcaatatgaagttctcagctcaatggtgggtgggcaatgactgccaactcttgaggccaatgaactcaggttaccccactcctyctcctcctgagttgctcactcactcctcattcactcaacattgattcagtagatatttgctacctgctctgtgccaggtaccaggtcagttgctgaaggagtaacagtgaacatgacggagtctttgtccccaaggagacccaaggtgtctcctagagccaggggcacattgcaagaccaaatatattcaacttacc

SEQ ID NO: 563 - DNA, 396 bp, Homo sapiens
ccatggagtcctgtcctctgttggggctcctactgatcctcttggcaatatgaagttctcagctcaatggtgggtgggcaatgactgccaactcttgaggccaatgaactcaggttaccccactcctcctcctcctgagttgctcactcactcctcattcactcaacattgattcagtagatatttgctacctgctctrtgccaggtaccaggtcagttgctgaaggagtaacagtgaacatgacggagtctttgtccccaaggagacccaaggtgtctcctagagccaggggcacattgcaagaccaaatatattcaacttaccaaaataatcatagacctagttctcaaaaagcaagaagactgattcctcgttgtcatttctcctcctcagca

SEQ ID NO: 564 - DNA, 396 bp, Homo sapiens
ttagagtctgtgggcccctccaagtgtggagtatggtgttacttcaccagagtttgaggagaaacattcttcttttggaaggccggggagcatagatggatatcaaggctgctgtttctaaaagcgaaacccaccaaacaacagtattagaatcatctgtggtgcttattaaagatacagattcctgggccccatcccmgacttatgaatcagaatctctgccagaggaagcctgagaatttgcattctcagatgattctgcattctcagataacacattctttaggtgattcttacacacactggagtttgggaatcgctgaaggctgttcacttctcttttctgagaaatgattcattcatttcagaaatatttgcagaggtccttatttattg

SEQ ID NO: 565 - DNA, 396 bp, Homo sapiens
tggcctcattcgtgtgataaatctgagccaccacgatatttgacttttcacaatttaatttatctgaaccctctattctctggctaaaaaatatcccttacttggacttctttattttattttcaattcccttaccagcactagcaggggactctgtactcatctgctggcgctgccataacaaagcactgcagcctgkggggctcaaaccacagaatttattctctcacagtcctagaggctagaagtccaagatcaaagtgtgggcagggtcggtttctcctgcagcctctctccttggcttatagagtgccaccttctacctgtgtcttcacatcatcacctcactgagcatgtctgtgtccaaatctccccttcttataagaccccagtcat

SEQ ID NO: 566 - DNA, 396 bp, Homo sapiens
tctccttggcttatagagtgccaccttctacctgtgtcttcacatcatcacctcactgagcatgtctgtgtccaaatctccccttcttataagaccccagtcatactggatgaggatccacccatatgagttcattttaccttaattatctctttaaacaccctgtctccaaatacagtcccattctgaggaactgagrgtaaagattcaacatatgaattttggaagggacctaattcagcccacaacaccctcttttgggatgtttattttcccccttaaggagctagttaggatgtcttatctcatgaacatgactgtgaacaggaaaacagggagagaatgaagctggccaaggaacagggctggtgtcagctagcagtgcttttctgatgt

SEQ ID NO: 567 - DNA, 396 bp, Homo sapiens
cattttaccttaattatctctttaaacaccctgtctccaaatacagtcccattctgaggaactgagagtaaagattcaacatatgaattttggaagggacctaattcagcccacaacaccctcttttgggatgtttattttcccccttaaggagctagttaggatgtcttatctcatgaacatgactgtgaacaggaaracagggagagaatgaagctggccaaggaacagggctggtgtcagctagcagtgcttttctgatgtgagtgggtcccacagggagcttgttaaaatgcagattctgattcattaggttccagagggacctgagatttcccatttctgacaagtttccagtgtgggggctgatgctgctggtccacggaccatactttg

SEQ ID NO: 568 - DNA, 396 bp, Homo sapiens
gggagagaatgaagctggccaaggaacagggctggtgtcagctagcagtgcttttctgatgtgagtgggtcccacagggagcttgttaaaatgcagattctgattcattaggttccagagggacctgagatttcccatttctgacaagtttccagtgtgggggctgatgctgctggtccacggaccatactttgagtakcaaggagcttgatacataatggctgagtgactttcagactcctgctgtagaaaaattatgagttggctgggcgtggtggctcacgcctgtaatcccagcactttgggaggccgaggtgggcagatcacctgaggtcaggagttcgagaccagcctggccaacatggtgaaacaccatctctaccaaaaatacaaaaa

SEQ ID NO: 569 - DNA, 396 bp, Homo sapiens
acttaagcccagaagactgaggttgcagtgagccgagattgcaccactgcactccagcttgggctacagagtgagactctatctcaaaaacaaagaaacaaacaacaacaataacaacaaaaaccaagtctctccctccactcaaaaatgcaagggcctgtctcccattgctgggtgcccaggtctcatgaatgtagayatgaattattccagtcagcctcaggagaatagaatgagccctcagatgccgaagcacctttcagattccaccggttttatcggctcatttaaacttcacttctaacacagtcctgcattacacacgtgtctgtcgttatgggcagctgcagagagggtcttaatggtcctaatgctcagtgaggatgcccaatggtc

SEQ ID NO: 570 - DNA, 396 bp, Homo sapiens
ctcaaaaacaaagaaacaaacaacaacaataacaacaaaaaccaagtctctccctccactcaaaaatgcaagggcctgtctcccattgctgggtgcccaggtctcatgaatgtagatatgaattattccagtcagcctcaggagaatagaatgagccctcagatgccgaagcacctttcagattccaccggttttatcrgctcatttaaacttcacttctaacacagtcctgcattacacacgtgtctgtcgttatgggcagctgcagagagggtcttaatggtcctaatgctcagtgaggatgcccaatggtcaacagaacctgccatcttcaggccatcaaggagctctggagttaaggaaatcatgagagcacagaggggcgggtacagcaga

SEQ ID NO: 571 - DNA, 396 bp, Homo sapiens
tgtagatatgaattattccagtcagcctcaggagaatagaatgagccctcagatgccgaagcacctttcagattccaccggttttatcggctcatttaaacttcacttctaacacagtcctgcattacacacgtgtctgtcgttatgggcagctgcagagagggtcttaatggtcctaatgctcagtgaggatgcccartggtcaacagaacctgccatcttcaggccatcaaggagctctggagttaaggaaatcatgagagcacagaggggcgggtacagcagagccctcgtggtaatgggttttgaggtctaggctctcttcacttgggtttgaaataagttcaatgactagtaatagctgagacacttctacccttcaaatgaagtaaatgg

SEQ ID NO: 572 - DNA, 396 bp, Homo sapiens
agcacctttcagattccaccggttttatcggctcatttaaacttcacttctaacacagtcctgcattacacacgtgtctgtcgttatgggcagctgcagagagggtcttaatggtcctaatgctcagtgaggatgcccaatggtcaacagaacctgccatcttcaggccatcaaggagctctggagttaaggaaatcawgagagcacagaggggcgggtacagcagagccctcgtggtaatgggttttgaggtctaggctctcttcacttgggtttgaaataagttcaatgactagtaatagctgagacacttctacccttcaaatgaagtaaatgggaaaatggagcattgttgagtccagggagctataatttaaaccccatatatctaaaagg

SEQ ID NO: 573 - DNA, 396 bp, Homo sapiens
cacacgtgtctgtcgttatgggcagctgcagagagggtcttaatggtcctaatgctcagtgaggatgcccaatggtcaacagaacctgccatcttcaggccatcaaggagctctggagttaaggaaatcatgagagcacagaggggcgggtacagcagagccctcgtggtaatgggttttgaggtctaggctctcttcrcttgggtttgaaataagttcaatgactagtaatagctgagacacttctacccttcaaatgaagtaaatgggaaaatggagcattgttgagtccagggagctataatttaaaccccatatatctaaaaggggtaacatttttgtgtgtgtgaaattggtgtcattcgcactgcatctacagttttctttttccttctc

SEQ ID NO: 574 - DNA, 396 bp, Homo sapiens
acatatttgggaaacgcatcatactcttcctgttcctcatgtccgttgctggcatattcaactattacctcatcttctttttcggaagtgactttgaaaactacataaagacgatctccaccaccatctcccctctacttctcattccctaactctctgctgaatatggggttggtgttctcatctaatcaatacctayaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttcttcactta

SEQ ID NO: 575 - DNA, 396 bp, Homo sapiens
tggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccrtgcattttgtttgtttcttcacttatcctgttctctgaagatgttttgtgaccaggtttgtgttttcttaaaataaaatgcagagacatgttttaagctgatagttgaggggttttgttaatggcttttgggggatttatctctatacccacaaacgactagtttgttttcctcaaactaaatgataatattaaaaa

SEQ ID NO: 576 - DNA, 396 bp, Homo sapiens
ttatctctatacccacaaacgactagtttgttttcctcaaactaaatgataatattaaaaatacacatcctggccaggtgtggtggctcatacctgtaatcccagcactttgggaggccgaggcaggtggatcacttgaggtcaggaattaagaccagcctggccaatatggtgaaagcctgtctgtactaaaaatacraaaattagccaggtatgctggtggatgcttataatcccagctacttgggaggttgaggcaggagaattgcttgaacccgggaggtagaggttgcagtgagccaagatcatgccactgcactccagcttgggcaacagagtgagactccatctcaaattaaaaaaaatacacatctggcttctggaaaaattacttga

SEQ ID NO: 577 - DNA, 396 bp, Homo sapiens
gatcatgccactgcactccagcttgggcaacagagtgagactccatctcaaattaaaaaaaatacacatctggcttctggaaaaattacttgaagatcttttatgacatccatccctcttcacacagccatgtgaattaggttggtatcttcatatactagcatcgtgcccagcacttccatgttatacagtttaaaakgttctgtaattccctgtgggaacctaagataatgcgaggaccgtcatacgtgcccccaaatattggcaaaccaatgaataaatgaatgaatgagtttatgaatcgctaactggctgtatttaatgaagtatgtgtgttgagccatttcccacagtgtggacagatttgtcccacaatatgggcctcttcccaaaggc

SEQ ID NO: 578 - DNA, 396 bp, Homo sapiens
aattaaaaaaaatacacatctggcttctggaaaaattacttgaagatcttttatgacatccatccctcttcacacagccatgtgaattaggttggtatcttcatatactagcatcgtgcccagcacttccatgttatacagtttaaaatgttctgtaattccctgtgggaacctaagataatgcgaggaccgtcatacrtgcccccaaatattggcaaaccaatgaataaatgaatgaatgagtttatgaatcgctaactggctgtatttaatgaagtatgtgtgttgagccatttcccacagtgtggacagatttgtcccacaatatgggcctcttcccaaaggccctaccacctaatgccatcacactggggatttgatttcaacatgtgaatt

SEQ ID NO: 579 - DNA, 396 bp, Homo sapiens
agttcatagtgacagtgatccagccactgtcatgacaggtgccacttggcagaaacagcacagcttggaagatggcggggtgtagtcaagattccaggatccccaacagagaagccagctcttataggggagccattcatcaggattgaactctcaatcgagctggacagtaataggtgggtctgtgttattccccagrtgagtatcatgacagtcacaatcctaggaaggatgtgaagcctcccccagctctcctccagttgcctgcttgggcagcagagatgatggaatgtggagtctggcgtggtctgaggcctgaatccatgtgcctcatgtatgatgctcaggcaagaggatctctcaattcaagggagagggcctgaatgagccttgctt

SEQ ID NO: 580 - DNA, 396 bp, Homo sapiens
cttggcagaaacagcacagcttggaagatggcggggtgtagtcaagattccaggatccccaacagagaagccagctcttataggggagccattcatcaggattgaactctcaatcgagctggacagtaataggtgggtctgtgttattccccagatgagtatcatgacagtcacaatcctaggaaggatgtgaagcctyccccagctctcctccagttgcctgcttgggcagcagagatgatggaatgtggagtctggcgtggtctgaggcctgaatccatgtgcctcatgtatgatgctcaggcaagaggatctctcaattcaagggagagggcctgaatgagccttgctttccaggcctgtctgatggtccaggctgaagcccctcctggcttg

SEQ ID NO: 581 - DNA, 396 bp, Homo sapiens
ctggcgtggtctgaggcctgaatccatgtgcctcatgtatgatgctcaggcaagaggatctctcaattcaagggagagggcctgaatgagccttgctttccaggcctgtctgatggtccaggctgaagcccctcctggcttgcactgccagacctcatccagcaggagctccttggcattgactgcttcaggatagttscttctgctctgagtgctctctaaagagcagtgctctaccatccaagctgggcttttcttttcttcttgctgatagggaaggcatgggacattgcaggatggaagtggcccccaggccttctcatgcctgggcttggtttggaaggtggtcaggtgatcaataatcctgattggcctggcattgaggagttttcctgg

SEQ ID NO: 582 - DNA, 396 bp, Homo sapiens
tgctctctaaagagcagtgctctaccatccaagctgggcttttcttttcttcttgctgatagggaaggcatgggacattgcaggatggaagtggcccccaggccttctcatgcctgggcttggtttggaaggtggtcaggtgatcaataatcctgattggcctggcattgaggagttttcctgggatgtggtcctttcrgttttttaaaaattatttttattgatacacatatttgtaggtatttgtggggtgcatgtgatactttattatgtgtgtggattgtgtaatgatgaagtcagggcatttagggtcttcatcaccttgattatcatttctatgtgttgagaacatttcaagttctcagttccagctattttgaaatagacagtccattt

SEQ ID NO: 583 - DNA, 396 bp, Homo sapiens
gatactttattatgtgtgtggattgtgtaatgatgaagtcagggcatttagggtcttcatcaccttgattatcatttctatgtgttgagaacatttcaagttctcagttccagctattttgaaatagacagtccattttgttagctacagtcacccaacccggctgtcagacattggaacttactcctattgaactgtrtatttgtacccattcaccaaactctctttgggctttcagttttacaactgggatgatcctgggaaaactaaagtaaatcagacacccgacgtgtgagctaggttataatatgcccagtggaccctggggacatcttagctttcagaggtcatgctgtccaagctgactgtggggcttccagaaggtggggagaggaa

SEQ ID NO: 584 - DNA, 396 bp, Homo sapiens
tatgtgtgtggattgtgtaatgatgaagtcagggcatttagggtcttcatcaccttgattatcatttctatgtgttgagaacatttcaagttctcagttccagctattttgaaatagacagtccattttgttagctacagtcacccaacccggctgtcagacattggaacttactcctattgaactgtgtatttgtacycattcaccaaactctctttgggctttcagttttacaactgggatgatcctgggaaaactaaagtaaatcagacacccgacgtgtgagctaggttataatatgcccagtggaccctggggacatcttagctttcagaggtcatgctgtccaagctgactgtggggcttccagaaggtggggagaggaaatgatgcaat

SEQ ID NO: 585 - DNA, 396 bp, Homo sapiens
tgggaaaactaaagtaaatcagacacccgacgtgtgagctaggttataatatgcccagtggaccctggggacatcttagctttcagaggtcatgctgtccaagctgactgtggggcttccagaaggtggggagaggaaatgatgcaatggcccatcagaggcactacttggggcctggggccagagtgcatgtctaagscattaaggggaggggagagcagccttcataattatgaagaggagtctcaggtgcacagcttctgatgagggacagcttctaattgaagacagcattgtgtaatgctcaaactccctgtcttcagagtgcctgctgtatcccaccatcagttctgtgacttctccctaagcctcaattttgcatgtgttacattggga

SEQ ID NO: 586 - DNA, 396 bp, Homo sapiens
cctgcatagcaaattcttgcaaatgtagggactcaaaacaatataaatttattatctgacagtttttctgggtcagaggtcttactaggctgtaatcagagggcaaccaaagctgtgatctcagctgaagctcaggattctcttccaagctcactggttgttggcagaattcagttctttccagttggaagactaaagyctacagtcttcagtctctagaagccttttctctggcacaggtttctctacaacatggccatttatgtctttaaggccaataggagaacatgattagcatattttttttaagtgaactttagacccttttttaaaggcctatctgattaggccaggcccaagtgagctttaagtcaactgattagagatcttaattac

SEQ ID NO: 587 - DNA, 396 bp, Homo sapiens
ctgaagctcaggattctcttccaagctcactggttgttggcagaattcagttctttccagttggaagactaaagcctacagtcttcagtctctagaagccttttctctggcacaggtttctctacaacatggccatttatgtctttaaggccaataggagaacatgattagcatattttttttaagtgaactttagacycttttttaaaggcctatctgattaggccaggcccaagtgagctttaagtcaactgattagagatcttaattacatctgcaaagtcccttcatgtttaccgtataacataacttagtgaaaggagtgaaattgcaaccaggttctgcctgcactccacggaaggggattctgcagaagtgtgggtcacgggggggtta

SEQ ID NO: 588 - DNA, 396 bp, Homo sapiens
agaacatgattagcatattttttttaagtgaactttagacccttttttaaaggcctatctgattaggccaggcccaagtgagctttaagtcaactgattagagatcttaattacatctgcaaagtcccttcatgtttaccgtataacataacttagtgaaaggagtgaaattgcaaccaggttctgcctgcactccacrgaaggggattctgcagaagtgtgggtcacgggggggttattttgggattctgcctacgtcactgagtcaaaagaagctgaatggttgtgatgctgaggtttttgggcagcagcagtgtgtgtgtgtgagtgaattcatacgtatgaccacctgggaagaaaggaggctgtggtttcctccacctcctggcagacaga

SEQ ID NO: 589 - DNA, 396 bp, Homo sapiens
gggattacagacacacactgccacgcctggctaatttttgtatttttagtagagacgaggttttgccatgttggccaggctggtcttgaactcctgacctcaagtgatccgcccacctcagcctcccaaagtgctgggattacagacgtgagccaccattaaccatttttctatctcctgtgggaaagggcacagtgaragaacagatgaagctgagacatacaagtgaactcctccctcctctccatttagactaaaataggattattcatactgagattctccctggttgcaaagagataatctgtgcaactgggtttttacaattatccctaccctatgctttcctcatctgtcttcctcgtagtcagctcaggctgctataacaaaacacca

SEQ ID NO: 590 - DNA, 396 bp, Homo sapiens
ggcagattcggtgtctaatgaggtcctgctttccagtttatagacagtgccttatcgctaccgccttacacagtggaaggagaggacgagaagctccttgggcttttttttgtttctttctttctctctctctctcttttttttttttttaataaggtcactatcttagtccattttgtgttgctaaaaggaacatctraggttgagtaatttattttattttaaaaagtggccaggcatggaggcttatcctgtaaccctaatcctttaggaggccaaaacagcaggattgtttgaggccaggagttcaagaccagcctaggcaagatagtgagaccccatctaccccatctctactaaaattttaaaaaattagctgtgtgttgtaaagtgtgc

SEQ ID NO: 591 - DNA, 396 bp, Homo sapiens
aatttattttattttaaaaagtggccaggcatggaggcttatcctgtaaccctaatcctttaggaggccaaaacagcaggattgtttgaggccaggagttcaagaccagcctaggcaagatagtgagaccccatctaccccatctctactaaaattttaaaaaattagctgtgtgttgtaaagtgtgcttgtagtcccrgccacttgagaggctgaggtgggtggagttcaaggctgcagtgagttatgattgagccactgcactccaacccgggtaacggggcaagaccttgtctctatttaaaaaaaaaaaatctttatgtggctcactattctgggtggctggaaagttcaagattgggcatctgcatctggtgacagcctcatgtcgcttcc

SEQ ID NO: 592 - DNA, 396 bp, Homo sapiens
taaccctaatcctttaggaggccaaaacagcaggattgtttgaggccaggagttcaagaccagcctaggcaagatagtgagaccccatctaccccatctctactaaaattttaaaaaattagctgtgtgttgtaaagtgtgcttgtagtcccggccacttgagaggctgaggtgggtggagttcaaggctgcagtgagwtatgattgagccactgcactccaacccgggtaacggggcaagaccttgtctctatttaaaaaaaaaaaatctttatgtggctcactattctgggtggctggaaagttcaagattgggcatctgcatctggtgacagcctcatgtcgcttccagtcatgggggaagacgaaggagagctggcacgtgcagatatcacg

SEQ ID NO: 593 - DNA, 396 bp, Homo sapiens
atcctttaggaggccaaaacagcaggattgtttgaggccaggagttcaagaccagcctaggcaagatagtgagaccccatctaccccatctctactaaaattttaaaaaattagctgtgtgttgtaaagtgtgcttgtagtcccggccacttgagaggctgaggtgggtggagttcaaggctgcagtgagttatgattragccactgcactccaacccgggtaacggggcaagaccttgtctctatttaaaaaaaaaaaatctttatgtggctcactattctgggtggctggaaagttcaagattgggcatctgcatctggtgacagcctcatgtcgcttccagtcatgggggaagacgaaggagagctggcacgtgcagatatcacgtgttgagg

SEQ ID NO: 594 - DNA, 396 bp, Homo sapiens
ttaaaaaattagctgtgtgttgtaaagtgtgcttgtagtcccggccacttgagaggctgaggtgggtggagttcaaggctgcagtgagttatgattgagccactgcactccaacccgggtaacggggcaagaccttgtctctatttaaaaaaaaaaaatctttatgtggctcactattctgggtggctggaaagttcargattgggcatctgcatctggtgacagcctcatgtcgcttccagtcatgggggaagacgaaggagagctggcacgtgcagatatcacgtgttgagggcagaagcgagagagagaggggagagatgccaggctctttttaacaaccagcactggggaaactaatagagtgagagctcactgactcctgagggaggacat

SEQ ID NO: 595 - DNA, 396 bp, Homo sapiens
atgggggaagacgaaggagagctggcacgtgcagatatcacgtgttgagggcagaagcgagagagagaggggagagatgccaggctctttttaacaaccagcactggggaaactaatagagtgagagctcactgactcctgagggaggacattaatctattgatgagcgacctgcctccatgacccaaacacctccaaygataccccacctccaacactgccacactagggattaactttcaacttgagatttagaggggggaaacttacaaactatcgcaggcactaataccactcatgagggctccaccttcatgacctaatcacttcctaaaggccttacctcttaatctcatcacattgaggattcgatttcaacttgaattttggggggac

SEQ ID NO: 596 - DNA, 396 bp, Homo sapiens
ctcgctgccacctgaaattagatcatttatttacccctttatttgttcagtttgccttgtccgttagaatataagcttccaaagggcaggagctttgcctatattgttaggccgggcatacaatgagcactcaaaaaaatatttgatgagtgtatgaaagaacagactgggttatgtaattgtgcctacttacctataygaccgtgtggtggggtttatggtgggtgtggtggtgatggctatagggctataagcaaatttgggacagggagtctaagaaatgttcttaaattttagtaagcaaagcatcctctacagaacctgtcttaaaacatgaaagttccttagtgctacccccagaggtatgatttggtaggtcaaggatagggcctggaa

SEQ ID NO: 597 - DNA, 396 bp, Homo sapiens
tgccacctgaaattagatcatttatttacccctttatttgttcagtttgccttgtccgttagaatataagcttccaaagggcaggagctttgcctatattgttaggccgggcatacaatgagcactcaaaaaaatatttgatgagtgtatgaaagaacagactgggttatgtaattgtgcctacttacctatatgaccrtgtggtggggtttatggtgggtgtggtggtgatggctatagggctataagcaaatttgggacagggagtctaagaaatgttcttaaattttagtaagcaaagcatcctctacagaacctgtcttaaaacatgaaagttccttagtgctacccccagaggtatgatttggtaggtcaaggatagggcctggaaattca

SEQ ID NO: 598 - DNA, 396 bp, Homo sapiens
cctgtcttaaaacatgaaagttccttagtgctacccccagaggtatgatttggtaggtcaaggatagggcctggaaattcacattcttgttaagatgttcttcatccggggtttgttgaccaccttttcagaagatttttgctctgtagctgtactacccaatgcagtagttcgtagtcagtgtggctcctgagccctygaagtgtagctcctctgaactgagacgtgctgtaaatgtaaattgcacaccggagtttgaagagttaatacaaagaaaaaggaatgcaaaacatctcattaataatgctttacactgattacatattgaaatggtaatcttgtagatatagtgcgttaaataaaatatactgttaggcttaatttcacgtctttata

SEQ ID NO: 599 - DNA, 396 bp, Homo sapiens
tcagccaatcaacaagagggcaaaagaacaaacatttgatgtgtaattacttaatttagtgcatatgcatttgggtcctcaatgtcagcactatggcaaccagaacatggccacaataactgtctggaaatgtctattcttacctggacccagcaggccatgccccactgattatataatctccctctctccttgttayggtctgaatgcttgcatccctcaaaaattcatgtgttgaaatcctaacccccaaggtgatgatattaggaggtcggccttttgagaggtaattaggtcatgaagacagcatcctcatgaatgggattagtgtccttataaaataggcccaagggagctcattcactttgtccaccatgtgagaacacagcgagaggg

SEQ ID NO: 600 - DNA, 396 bp, Homo sapiens
ccttgttacggtctgaatgcttgcatccctcaaaaattcatgtgttgaaatcctaacccccaaggtgatgatattaggaggtcggccttttgagaggtaattaggtcatgaagacagcatcctcatgaatgggattagtgtccttataaaataggcccaagggagctcattcactttgtccaccatgtgagaacacagygagagggcaccatttatgcaccaggaaatgggccttttccagacaatctgtcggtgcctggatcttggacttcacagcctctagaactgtgagaaattaatttgttttttataagccaccaaatctatggttttttttatagaaaccgtaatggactaaaacactccctaattatatttaaacttatcagtgcactg

SEQ ID NO: 601 - DNA, 396 bp, Homo sapiens
ctaacccccaaggtgatgatattaggaggtcggccttttgagaggtaattaggtcatgaagacagcatcctcatgaatgggattagtgtccttataaaataggcccaagggagctcattcactttgtccaccatgtgagaacacagcgagagggcaccatttatgcaccaggaaatgggccttttccagacaatctgtyggtgcctggatcttggacttcacagcctctagaactgtgagaaattaatttgttttttataagccaccaaatctatggttttttttatagaaaccgtaatggactaaaacactccctaattatatttaaacttatcagtgcactgggcagtgacatattaaaagaatgctggccaacgtaattgacaccataaggct

SEQ ID NO: 602 - DNA, 396 bp, Homo sapiens
tcatctcattttaaccttttgtttcaaagcctctcttttcatgacttccccgccttcatttttcccatatggtggggttattattaagacattaaatgagagtggacaggtaggcaaaggaggtgggttgcaggggagttgagggttgcctgtgtacttttctagactgttccacttcacatcagtgaaatattcccarttgatactatcatgaaacaaagcaaatgaaatgctgagcacggagcttcgtcttgatgaaatgctgaaagaaaagaaaggaaaaataaagtagccattatttttgcccttcctcccacccccatgtttactactcttatttctcttttgtattgttgtgttggaagcacagcatcagaaaaactcccagttttgaga

SEQ ID NO: 603 - DNA, 396 bp, Homo sapiens
acaggtaggcaaaggaggtgggttgcaggggagttgagggttgcctgtgtacttttctagactgttccacttcacatcagtgaaatattcccaattgatactatcatgaaacaaagcaaatgaaatgctgagcacggagcttcgtcttgatgaaatgctgaaagaaaagaaaggaaaaataaagtagccattatttttrcccttcctcccacccccatgtttactactcttatttctcttttgtattgttgtgttggaagcacagcatcagaaaaactcccagttttgagagataactcagtgtttagttcacttaaacctgagaaaggagaagaggatgccaccgtgaggtccaggacgtaaagaggaaaaaaacagacaaaaaaatccatatga

SEQ ID NO: 604 - DNA, 396 bp, Homo sapiens
caggtaggcaaaggaggtgggttgcaggggagttgagggttgcctgtgtacttttctagactgttccacttcacatcagtgaaatattcccaattgatactatcatgaaacaaagcaaatgaaatgctgagcacggagcttcgtcttgatgaaatgctgaaagaaaagaaaggaaaaataaagtagccattatttttgmccttcctcccacccccatgtttactactcttatttctcttttgtattgttgtgttggaagcacagcatcagaaaaactcccagttttgagagataactcagtgtttagttcacttaaacctgagaaaggagaagaggatgccaccgtgaggtccaggacgtaaagaggaaaaaaacagacaaaaaaatccatatgaa

SEQ ID NO: 605 - DNA, 396 bp, Homo sapiens
ttcgtcttgatgaaatgctgaaagaaaagaaaggaaaaataaagtagccattatttttgcccttcctcccacccccatgtttactactcttatttctcttttgtattgttgtgttggaagcacagcatcagaaaaactcccagttttgagagataactcagtgtttagttcacttaaacctgagaaaggagaagaggaygccaccgtgaggtccaggacgtaaagaggaaaaaaacagacaaaaaaatccatatgaaatgaaaatgtgaaagaggcgctttcgagcagatgagtgttgtagattacagtgttgagagctgtttgtgtccagagctgcttgctgcacctggcgggataaacactggtctaacagaggatccttgtttcaaggaggct

SEQ ID NO: 606 - DNA, 396 bp, Homo sapiens
aagaaaagaaaggaaaaataaagtagccattatttttgcccttcctcccacccccatgtttactactcttatttctcttttgtattgttgtgttggaagcacagcatcagaaaaactcccagttttgagagataactcagtgtttagttcacttaaacctgagaaaggagaagaggatgccaccgtgaggtccaggacrtaaagaggaaaaaaacagacaaaaaaatccatatgaaatgaaaatgtgaaagaggcgctttcgagcagatgagtgttgtagattacagtgttgagagctgtttgtgtccagagctgcttgctgcacctggcgggataaacactggtctaacagaggatccttgtttcaaggaggctgccttttatttggggggacaa

SEQ ID NO: 607 - DNA, 396 bp, Homo sapiens
attatttttgcccttcctcccacccccatgtttactactcttatttctcttttgtattgttgtgttggaagcacagcatcagaaaaactcccagttttgagagataactcagtgtttagttcacttaaacctgagaaaggagaagaggatgccaccgtgaggtccaggacgtaaagaggaaaaaaacagacaaaaaaayccatatgaaatgaaaatgtgaaagaggcgctttcgagcagatgagtgttgtagattacagtgttgagagctgtttgtgtccagagctgcttgctgcacctggcgggataaacactggtctaacagaggatccttgtttcaaggaggctgccttttatttggggggacaaaattgttcttgaaagctgctcagtggtt

SEQ ID NO: 608 - DNA, 396 bp, Homo sapiens
tttgtattgttgtgttggaagcacagcatcagaaaaactcccagttttgagagataactcagtgtttagttcacttaaacctgagaaaggagaagaggatgccaccgtgaggtccaggacgtaaagaggaaaaaaacagacaaaaaaatccatatgaaatgaaaatgtgaaagaggcgctttcgagcagatgagtgttrtagattacagtgttgagagctgtttgtgtccagagctgcttgctgcacctggcgggataaacactggtctaacagaggatccttgtttcaaggaggctgccttttatttggggggacaaaattgttcttgaaagctgctcagtggttcaagctacagcatggtggactagcagaatggactccagggcctccgagga

SEQ ID NO: 609 - DNA, 396 bp, Homo sapiens
ttttgagagataactcagtgtttagttcacttaaacctgagaaaggagaagaggatgccaccgtgaggtccaggacgtaaagaggaaaaaaacagacaaaaaaatccatatgaaatgaaaatgtgaaagaggcgctttcgagcagatgagtgttgtagattacagtgttgagagctgtttgtgtccagagctgcttgcygcacctggcgggataaacactggtctaacagaggatccttgtttcaaggaggctgccttttatttggggggacaaaattgttcttgaaagctgctcagtggttcaagctacagcatggtggactagcagaatggactccagggcctccgaggagacagtgactgctgccagaaatagtcaaggatagaaaggaagga

(19) United States
(12) Patent Application Publication
(10) Pub. No.: US 2005/0282855 A1
(43) Pub. Date: Dec. 22, 2005

(54) Susceptibility gene for myocardial infarction, stroke, and PAOD; methods of treatment

(75) Inventors: Anna Helgadottir, Reykjavik (IS); Mark Gurney, East Grand Rapids, MI (US); Jeffrey R. Gulcher, Lake Barrington, IL (US); Hakon Hakonarson, Reykjavik (IS)

Correspondence Address: MARSHALL, GERSTEIN & BORUN LLP, 233 S. WACKER DRIVE, SUITE 6300, SEARS TOWER, CHICAGO, IL, 60606, US

(73) Assignee: deCODE Genetics ehf., Reykjavik (IS)

(21) Appl. No.: 10/769,744

(22) Filed: Jan. 30, 2004

Related U.S. Application Data
(63) Continuation-in-part of application No. PCT/US03/32556, filed on Oct. 16, 2003.
(60) Provisional application No. 60/419,433, filed on Oct. 17, 2002.
(60) Provisional application No. 60/449,331, filed on Feb. 21, 2003.

(51) Int. Cl. A61K 31/47

(52) U.S. Cl. 514/311; 514/314

(57) ABSTRACT

Linkage of myocardial infarction (MI) and a locus on chromosome 13q12 is disclosed. In particular, the FLAP gene within this locus is shown by genetic association analysis to be a susceptibility gene for MI and ACS, as well as stroke and PAOD. Pathway targeting for treatment and diagnostic applications in identifying those who are at risk of developing MI, ACS, stroke or PAOD, in particular are described.

### RELATED APPLICATIONS

This application is a continuation-in-part of International Application No. PCT/US03/32556, which designated the United States and was filed on Oct. 16, 2003, published in English, which claims the benefit of U.S. Provisional Application No. 60/419,433, filed on Oct. 17, 2002 and U.S. Provisional Application No. 60/449,331, filed on Feb. 21, 2003. The entire teachings of the above applications are incorporated herein by reference.

### BACKGROUND OF THE INVENTION

Myocardial infarction (MI) and Acute Coronary Syndrome (ACS), e.g., unstable angina, non-ST-elevation myocardial infarction (NSTEMI) or ST-elevation myocardial infarction (STEMI), are the leading causes of hospital admissions in industrialized countries. Cardiovascular disease continues to be the principle cause of death in the United States, Europe and Japan. The costs of the disease are high both in terms of morbidity and mortality, as well as in terms of the financial burden on health care systems.

Myocardial infarction generally occurs when there is an abrupt decrease in coronary blood flow following a thrombotic occlusion of a coronary artery previously damaged by atherosclerosis. In most cases, infarction occurs when an atherosclerotic plaque fissures, ruptures or ulcerates and when conditions favor thrombogenesis. In rare cases, infarction may be due to coronary artery occlusion caused by coronary emboli, congenital abnormalities, coronary spasm, and a wide variety of systemic, particularly inflammatory diseases. Medical risk factors for MI include cigarette smoking, diabetes, hypertension and serum total cholesterol levels>200 mg/dL, elevated serum LDL cholesterol, and low serum HDL cholesterol. Event rates in individuals without a prior history of cardiovascular disease are about 1%. In individuals who have had a first MI or ACS, the risk of a repeat MI within the next year is 10-14%, despite maximal medical management including angioplasty and stent placement.

Atherosclerosis can affect vascular beds in many large and medium arteries. Myocardial infarction and unstable angina (acute coronary syndrome (ACS)) stem from coronary artery atherosclerosis, while ischemic stroke most frequently is a consequence of carotid or cerebral artery atherosclerosis. Limb ischemia caused by peripheral arterial occlusive disease (PAOD) may occur as a consequence of iliac, femoral and popliteal artery atherosclerosis. The atherosclerotic diseases remain common despite the wide-spread use of medications that inhibit thrombosis (aspirin) or treat medical risk factors such as elevated cholesterol levels in blood (statins), diabetes, or hypertension (diuretics and anti-hypertensives).

Atherosclerotic disease is initiated by the accumulation of lipids within the artery wall, and in particular, the accumulation of low-density lipoprotein (LDL) cholesterol. The trapped LDL becomes oxidized and internalized by macrophages. This causes the formation of atherosclerotic lesions containing accumulations of cholesterol-engorged macrophages, referred to as “foam cells”. As disease progresses, smooth muscle cells proliferate and grow into the artery wall forming a “fibrous cap” of extracellular matrix enclosing a lipid-rich, necrotic core. Present in the arterial walls of most people throughout their lifetimes, fibrous atherosclerotic plaques are relatively stable. Such fibrous lesions cause extensive remodeling of the arterial wall, outwardly displacing the external, elastic membrane, without reduction in luminal diameter or serious impact on delivery of oxygen to the heart. Accordingly, patients can develop large, fibrous atherosclerotic lesions without luminal narrowing until late in the disease process. However, the coronary arterial lumen can become gradually narrowed over time and in some cases compromise blood flow to the heart, especially under high demand states such as exercise. This can result in reversible ischemia causing chest pain relieved by rest called stable angina.

In contrast to the relative stability of fibrous atherosclerotic lesions, the culprit lesions associated with myocardial infarction and unstable angina (each of which are part of the acute coronary syndrome) are characterized by a thin fibrous cap, a large lipid core, and infiltration of inflammatory cells such as T-lymphocytes and monocyte/macrophages. Non-invasive imaging techniques have shown that most MI's occur at sites with low- or intermediate-grade stenoses, indicating that coronary artery occlusion is due most frequently to rupture of culprit lesions with consequent formation of a thrombus or blood clot and not solely due to luminal narrowing by stenosis. Plaque rupture may be due to erosion or uneven thinning of the fibrous cap, usually at the margins of the lesion where macrophages enter, accumulate, and become activated by a local inflammatory process. Thinning of the fibrous cap may result from degradation of the extracellular matrix by proteases released from activated macrophages. These changes producing plaque instability and risk of MI may be augmented by production of tissue-factor procoagulant and other factors increasing the likelihood of thrombosis.

In acute coronary syndrome, the culprit lesion showing rupture or erosion with local thrombosis typically is treated by angioplasty or by balloon dilation and placement of a stent to maintain luminal patency. Patients experiencing ACS are at high risk for a second coronary event due to the multi-vessel nature of coronary artery disease with event rates approaching 10-14% within 12 months after the first incident.

The emerging view of MI is as an inflammatory disease of the arterial vessel wall on preexisting chronic atherosclerotic lesions, sometimes triggering rupture of culprit lesions and leading to local thrombosis and subsequent myocardial infarction. The process that triggers and sustains arterial wall inflammation leading to plaque instability is unknown, however, it results in the release into the circulation of tumor necrosis factor alpha and interleukin-6. These and other cytokines or biological mediators released from the damaged vessel wall stimulate an inflammatory response in the liver causing elevation in several non-specific general inflammatory markers including C-reactive protein. Although not specific to atherosclerosis, elevated C-reactive protein (CRP) and serum amyloid A appear to predict risk for MI, perhaps as surrogates for vessel wall inflammation.

Although classical risk factors such as smoking, hyperlipidemia, hypertension, and diabetes are associated with many cases of coronary heart disease (CHD) and MI, many patients do not have involvement of these risk factors. In fact, many patients who exhibit one or more of these risk factors do not develop MI. Family history has long been recognized as one of the major risk factors. Although some of the familial clustering of MI reflects the genetic contribution to the other conventional risk factors, a large number of studies have suggested that there are significant genetic susceptibility factors, beyond those of the known risk factors (Friedlander Y, et al., Br. Heart J. 1985; 53:382-7, Shea S. et al., J. Am. Coll. Cardiol. 1984; 4:793-801, and Hopkins P. N., et al., Am. J. Cardiol. 1988; 62:703-7). Major genetic susceptibility factors have only been identified for the rare Mendelian forms of hyperlipidemia such as a familial hypercholesterolemia.

Genetic risk is conferred by subtle differences in genes among individuals in a population. Genes differ between individuals most frequently due to single nucleotide polymorphisms (SNP), although other variations are also important. SNP are located on average every 1000 base pairs in the human genome. Accordingly, a typical human gene containing 250,000 base pairs may contain 250 different SNP. Only a minor number of SNP are located in exons and alter the amino acid sequence of the protein encoded by the gene. Most SNP have no effect on gene function, while others may alter transcription, splicing, translation, or stability of the mRNA encoded by the gene. Additional genetic polymorphism in the human genome is caused by insertion, deletion, translocation, or inversion of either short or long stretches of DNA. Genetic polymorphisms conferring disease risk may therefore directly alter the amino acid sequence of proteins, may increase the amount of protein produced from the gene, or may decrease the amount of protein produced by the gene.

As genetic polymorphisms conferring risk of disease are uncovered, genetic testing for such risk factors is becoming important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia.

Many general inflammatory markers predict risk of coronary heart disease, although these markers are not specific to atherosclerosis. For example, Stein (Stein, S., Am J Cardiol, 87 (suppl):21A-26A (2001)) discusses the use of any one of the following serum inflammatory markers as surrogates for predicting risk of coronary heart disease including C-reactive protein (CRP), serum amyloid A, fibrinogen, interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, and matrix metalloprotease type-9. Elevation in one more of these serum inflammatory markers is not specific to coronary heart disease but also occurs with age or in association with cerebrovascular disease, peripheral vascular disease, non-insulin dependent diabetes, osteoarthritis, bacterial infection, and sepsis.

Serum C-reactive protein (CRP) is viewed as a convenient and sensitive marker of systemic inflammation. Generally CRP is measured in serum samples using commercially available enzyme-linked immunosorbent assays (EIA). Consistent across multiple published studies is the finding of a correlation between increased risk for coronary artery disease with increased serum CRP. For example, in the Women's Health Study, CRP was measured in 27,939 apparently healthy American women. The cut-off points for quintiles of serum CRP in women were: less than or equal to 0.49, more than 0.49 to 1.08, more than 1.08 to 2.09, more than 2.09 to 4.19, and more than 4.19 mg CRP per liter, see Ridker, P. M. et al., New England J. Med., 347: 1557-1565 (2001). In comparison to the lowest quintile, and even when adjusting for age, every quintile more than 0.49 mg CRP per liter was associated with increased risk for coronary heart disease with the highest relative risk of 4.5 seen for those women in the highest quintile of serum CRP (more than 4.19 mg CRP per liter). A similar correlation between increased serum CRP and increased risk for coronary heart disease in women has been reported (Ridker, P. M. et al., New Engld. J. Med., 342:836-843 (2000) and Bermudez, E. A. et. al., Arterioscler. Thromb. Vasc. Biol., 22: 1668-1673 (2002)). Men also show a correlation between increased serum inflammatory markers such as CR and increased risk for coronary heart disease has been reported (Doggen, C. J. M. et al., J. Internal Med., 248:406-414 (2000) and Ridker, P. M. et al., New England J. Med., 336: 973-979 (1997)). Quintiles for serum CRP as reported by Doggen et al., were less than 0.65, more than 0.65 to 1.18, more than 1.18 to 2.07, more than 2.07 to 4.23, and more than 4.23 mg CRP per liter. Unlike women, elevated serum CRP correlates with increased relative risk for coronary heart disease only in the 4^{th} and 5^{th} quintiles of CRP (relative risk of 1.7× and 1.9×, respectively).

Serum CRP in women also has been measured in conjunction with lipid markers such as levels of serum low density lipoprotein-cholesterol (LDL-C). In the study by Ridker, P. M. et al. (2002), serum CRP and LDL-C are minimally correlated, screening for both serum markers provided better prognostic indication than either alone. Thus, women with serum CRP above median values (more than 1.52 mg CRP per liter) and also serum LDL-C above median values (more than 123.7 mg LDL-C per deciliter) were at highest risk for coronary heart disease.

Elevated CRP or other serum inflammatory markers is also prognostic for increased risk of a second myocardial infarct in patients with a previous myocardial infarct (Retterstol, L. et al., Atheroscler., 160: 433-440 (2002)).

Since CRP is produced in the liver, there is no a priori mechanistic explanation for why elevation in CRP and other serum inflammatory markers should be prognostic for coronary artery disease. As discussed by Doggen, C. J. M., et al., one or more of the following factors were speculated to account for the correlation observed: (1) intrinsic inflammation and tissue damage within arterial lesions, (2) prior infection by Helicobacter pylori or by Chlamydia pneumoniae, (3) release of peptide cytokines including interleukin-6, or (4) activation of the complement system.

The end products of the leukotriene pathway are potent inflammatory lipid mediators derived from arachidonic acid. They can potentially contribute to development of atherosclerosis and destabilization of atherosclerotic plaques through lipid oxidation and/or proinflammatory effects. LTC4, LTD4, and LTE4, are known to induce vasoconstriction. Allen et al., Circulation, 97:2406-2413 (1998) described a novel mechanism in which atherosclerosis is associated with the appearance of a leukotriene receptor(s) capable of inducing hyperactivity of human epicardial coronary arteries in response to LTC4 and LTD4. LTB4, on the other hand, is a strong proinflammatory agent. Increased production of these end products, of the leukotriene pathway, could therefore serve as a risk factor for MI and atherosclerosis, whereas both inflammation and vasoconstriction/vasospasm have a well established role in the pathogenesis of MI and atherosclerosis. It has also been shown that a heterozygous deficiency of the 5-LO enzyme in a knockout mouse model decreases atherosclerotic lesion size in LDLR−/− mice by about 95%. (Mehrabian et al., Circulation Research. 91:120 (2002)). However, such genetic evidence for leukotriene involvement in MI or atherosclerosis in humans has not been reported. Mehrabian et al. did report a very small genetic association study looking for correlation between promoter polymorphisms of 5-LO and carotid intimal thickening in normal individuals. However, their data paradoxically suggest that a lower amount of leukotriene production correlates with carotid atherosclerosis.

### SUMMARY OF THE INVENTION

As described herein, a gene on chromosome 13q12 has been identified as playing a major role in myocardial infarction (MI). This gene, herein after referred to as the MI gene, comprises nucleic acid that encodes 5-lipoxygenase activating protein (ALOX5AP or FLAP,) herein after referred to as FLAP. The gene has also been shown to play a role in stroke and PAOD.

The invention pertains to methods of treatment (prophylactic and/or therapeutic) for certain diseases and conditions (e.g., MI, ACS, atherosclerosis, stroke, PAOD) associated with FLAP or with other members of the leukotriene pathway (e.g., biosynthetic enzymes such as FLAP, arachidonate 4-lipoxygenase (5-LO), leukotriene C4 synthetase (LTC4S), leukotriene A4 hydrolase (LTA4H), leukotriene B4 12-hydroxydehydrogenase (LTB4DH)); receptors and/or binding agents of the enzymes; and receptors for the leukotrienes LTA4, LTB4, LTC4, LTD4, LTE4, Cys LT1, Cys LT2, including leukotriene B4 receptor 1 (BLT1), leukotriene B4 receptor 2 (BLT2), cysteinyl leukotriene receptor 1 (CysLTR1), cysteinyl leukotriene receptor 2 (CysLTR2). The methods include the following: methods of treatment for myocardial infarction or susceptibility to myocardial infarction; methods of treatment for transient ischemic attack, transient monocular blindness or stroke, or susceptibility to stroke; methods of treatment for claudication, PAOD or susceptibility to PAOD; methods of treatment for acute coronary syndrome (e.g., unstable angina, non-ST-elevation myocardial infarction (NSTEMI) or ST-elevation myocardial infarction (STEMI)); methods for reducing risk of MI, stroke or PAOD in persons with asymptomatic ankle/brachial index less than 0.9; methods for decreasing risk of a second myocardial infarction or stroke; methods of treatment for atherosclerosis, such as for patients requiring treatment (e.g., angioplasty, stents, revascularization procedure) to restore blood flow in arteries (e.g., coronary, carotid, and/or femoral arteries); methods of treatment for asymptomatic ankle/brachial index of less than 0.9; and/or methods for decreasing leukotriene synthesis (e.g., for treatment of myocardial infarction, stroke or PAOD).

In the methods of the invention, a leukotriene synthesis inhibitor is administered to an individual in a therapeutically effective amount. The leukotriene synthesis inhibitor can be an agent that inhibits or antagonizes a member of the leukotriene synthesis pathway (e.g., FLAP, 5-LO, LTC4S, LTA4H, and LTB4DH). For example, the leukotriene synthesis inhibitor can be an agent that inhibits or antagonizes FLAP polypeptide activity (e.g., a FLAP inhibitor) and/or FLAP nucleic acid expression, as described herein (e.g., a FLAP nucleic acid antagonist). In another embodiment, the leukotriene synthesis inhibitor is an agent that inhibits or antagonizes polypeptide activity and/or nucleic acid expression of another member of the leukotriene biosynthetic pathway (e.g., LTC4S, LTA4H, LTB4DH). In preferred embodiments, the agent alters activity and/or nucleic acid expression of FLAP or of 5-LO. Preferred agents include those set forth in the Agent Table herein. In another embodiment, preferred agents can be: 1-((4-chlorophenyl)methyl)-3-((1,1-dimethylethyl)thio)-alpha,alpha-dimethyl-5-(2-quinolinylmethoxy)-1H-Indole-2-propanoic acid otherwise known as MK-0591, (R)-(+)-alpha-cyclopentyl-4-(2-quinolinylmethoxy)-Benzeneacetic acid otherwise known as BAY-x-1005, 3-(3-(1,1-dimethylethylthio-5-(quinoline-2-ylmethoxy)-1-(4-chloromethylphenyl)indole-2-yl)-2,2-dimethylpropionaldehyde oxime-0-2-acetic acid otherwise known as A-81834, optically pure enantiomers, salts, chemical derivatives, and analogues; or can be zileuton, atreleuton, 6-((3-fluoro-5-(tetrahydro-4-methoxy-2H-pyran-4yl)phenoxy)methyl)-1-methyl-2(1H)-quinlolinone otherwise known as ZD-2138, 1-((4-chlorophenyl)methyl)-3-((1,1dimethylethyl)thio)-alpha,alpha-dimethyl-5-(2-quinolinylmethoxy)-1H-Indole-2-propanoic acid otherwise known as MK-886, 4-(3-(4-(2-Methyl-imidazol-1-yl)-phenylsulfanyl)-phenyl)-tetrahydro-pyran-4-carboxylic acid amide otherwise known as CJ-13610, their optically pure enantiomers, salts, chemical derivatives, and analogues. In another embodiment, the agent alters metabolism or activity of a leukotriene (e.g., LTA4, LTB4, LTC4, LTD4, LTE4, Cys LT1, Cys LT2), such as leukotriene antagonists or antibodies to leukotrienes, as well as agents which alter activity of a leukotriene receptor (e.g., BLT1, BLT2, CysLTR1, and CysLTR2).

In certain embodiments of the invention, the individual is an individual who has at least one risk factor, such as an at-risk haplotype for myocardial infarction, stroke or PAOD; an at-risk haplotype in the FLAP gene; a polymorphism in a FLAP nucleic acid; an at-risk polymorphism in the 5-LO gene promoter, diabetes; hypertension; hypercholesterolemia; elevated triglycerides; elevated lp(a); obesity; ankle/brachial index (ABI) less than 0.9; a past or current smoker; transient ischemic attack; transient monocular blindness; carotid endarterectomy; asymptomatic carotid stenosis; claudicatioin; limb ischemia leading to gangrene, ulceration or amputation; a vascular or peripheral aratery revascularization graft; an elevated inflammatory marker (e.g., a marker such as C-reactive protein (CRP), serum amyloid A, fibrinogen, a leukotriene, a leukotriene metabolite, interleukin-6, tissue necrosis factor-alpha, a soluble vascular cell adhesion molecule (sVCAM), a soluble intervascular adhesion molecule (sICAM), E-selectin, matrix metalloprotease type-1, matrix metalloprotease type-2, matrix metalloprotease type-3, matrix metalloprotease type-9, myeloperoxidase (MPO), and N-tyrosine); increased LDL cholesterol and/or decreased HDL cholesterol; increased leukotriene synthesis; and/or at least one previous myocardial infarction, ACS, stable angina, previous transient ischemic attack, transient monocular blindness, or stroke, asymptomatic carotid stenosis or carotid endarterectomy, atherosclerosis, requires treatment for restoration of coronary artery blood flow (e.g., angioplasty, stent, revascularization procedure).

The invention additionally pertains to methods of assessing an individual for an increased risk of MI, ACS, atherosclerosis, stroke, or PAOD, by assessing a level of a leukotriene metabolite (e.g., LTE4, LTD4, LTB4) in the individual (e.g., in a sample of blood, serum, plasma or urine). An increased level of leukotriene metabolite is indicative of an increased risk. The invention also encompasses methods of assessing an individual for an increased risk of MI, ACS, atherosclerosis, stroke, transient ischemic attack, transient monocular blindness, asymptomatic carotid stenosis, PAOD, claudication, or limb ischemia, by stimulating production of a leukotriene or a leukotriene metabolite in a test sample from the individual (e.g., a sample comprising neutrophils), using a calcium ionophore, and comparing the level of the leukotriene or leukotriene metabolite with a control level. A level of production of the leukotriene or leukotriene metabolite that is significantly greater than the control level, is indicative of increased risk.

The invention further pertains to methods of assessing response to treatment with a leukotriene synthesis inhibitor, by assessing a level of a leukotriene or leukotriene metabolite in the individual before treatment, and comparing the level to a level of the leukotriene or leukotriene metabolite assessed during or after treatment. A level that is significantly lower during or after treatment, than before treatment, is indicative of efficacy of the treatment with the leukotriene synthesis inhibitor. The invention additionally pertains to methods of assessing response to treatment with a leukotriene synthesis inhibitor, by stimulating production of a leukotriene or a leukotriene metabolite in a first test sample from the individual (e.g., a sample comprising neutrophils) before treatment, using a calcium ionophore, and comparing the level of the leukotriene or leukotriene metabolite with a level of production of the leukotriene or leukotriene in a second test sample from the individual, during or after treatment. A level of production of the leukotriene or leukotriene metabolite in the second test sample that is significantly lower than the level in the first test sample, is indicative of efficacy of the treatment. Similarly, the invention encompasses methods of assessing response to treatment with a leukotriene synthesis inhibitor, by assessing a level of an inflammatory marker in the individual before treatment, and during or after treatment. A level of the inflammatory marker during or after treatment, that is significantly lower than the level of inflammatory marker before treatment, is indicative of efficacy of the treatment.

The invention also pertains to use of leukotriene synthesis inhibitors for the manufacture of a medicament for the treatment of MI, ACS, stroke, PAOD, and/or atherosclerosis, as described herein, as well as for the manufacture of a medicament for the reduction of leukotriene synthesis.

### BRIEF DESCRIPTION OF THE DRAWINGS

The foregoing and other objects, features and advantages of the invention will be apparent from the following more particular description of preferred embodiments of the invention. The patent or application file contains at least one drawing executed in color. Copies of this patent or patent application publication with color drawing(s) will be provided by the Office upon request and payment of the necessary fee.

FIG. 1 shows the multipoint non-parametric LOD scores of a linkage scan of 160 female patients in large extended pedigrees and genotyped using a 1000 framework map on chromosome 13. A LOD score suggestive of linkage of 2.5 was found at marker D13S289. The marker map for chromosome 13 that was used in the linkage analysis is shown in Table 1.

FIG. 2 shows LOD score results for the families after adding 14 additional markers to the candidate region. The inclusion of additional microsatellite markers increased the information on sharing by decent from 0.7 to 0.8, around the markers that gave the highest LOD scores. The marker map used in the second step of linkage anaysis is shown in Table 2.

FIG. 3.1 shows the results from a haplotype association case-control analysis of 437 female MI patients versus 721 controls using combinations 4 and 5 microsatellite markers to define the test haplotypes. The p-value of the association is plotted on the y-axis and position of markers on the x-axis. Only haplotypes that show association with a p-value <10⁻⁵ are shown in the figure. The most significant microsatellite marker haplotype association is found using markers DG13S1103, DG13S166, DG13S1287, DG13S1061 and DG13S301, with alleles 4, 0,2, 14 and 3, respectively (p-value of 1.02×10⁻⁷). Carrier frequency of the haplotype is 7.3% in female MI patients and 0.3% in controls. The segment that is common to all the haplotypes shown in the figure includes only one gene, FLAP.

FIG. 3.2 shows the alleles of the markers defining the most significant microsatellite marker haplotypes. The segment defined with a black square is common to all the of most significantly associated haplotypes. The FLAP nucleic acid is located between makers DG13S166 and D13S1238. Two marker haplotype involving alleles 0 and −2 for markers DG13S166 and D13S1238, respectively, is found in excess in patients. Carrier frequency of this haploype is 27% in patients and 15.4% in controls (p-value 1×10⁻³). Therefore, association analysis confirms that the most tightly MI-associated gene within the linkage peak is FLAP.

FIG. 4 shows the markers and genes around the FLAP (ALOX5AP) gene.

FIG. 5 shows the relative location of key SNPs and exons of the ALOX5AP/FLAP gene (exons shown in vertical rectangles). Haplotype length varies between 33 to 68 kb.

FIG. 6.1-6.82 show the genomic sequence of the FLAP gene (SEQ ID NO: 1).

FIG. 7 shows the amino acid sequence of FLAP (SEQ ID NO:2) and the mRNA of FLAP (SEQ ID NO: 3).

FIG. 8.1-8.40 show the sequences of the FLAP nucleic acid flanking the SNPs that were identified by sequencing samples from patients (SEQ ID NOs: 398-535).

FIG. 9 shows a significant positive correlation between serum LTE4 levels and serum CRP levels.

FIG. 10 depicts LTB4 production of ionomycin stimulated neutrophils from MI patients (n=41) and controls (n=35). The log-transformed (mean+SD) values measured at 15 and 30 minutes of stimulated cells are shown. (a) LTB4 production in MI patients and controls. The difference in the mean values between patients and the controls is tested using a two-sample t-test of the log-transformed values. (b) LTB4 production in MI male carriers (red bars) and non-carriers (white bars) of HapA. Mean values of controls (blue bars) are included for comparison. Of note, males with the HapA produce highest amounts of LTB4 (p<0.005 compared to controls). (c). Schematic representation of the 5-LO pathway with leukotriene bioactive products.

FIG. 11 shows a genome wide linkage scan using 1,000 microsatellite markers for all (black) (n=713), female (red), (n=140), male (blue) (n=575), and early onset MI patients (green) (n=194). The LOD score is expressed on the y axis and the distance from the pter in Kosambi cM on the x axis.

FIG. 12 shows a schematic view of the chromosome 13 linkage region showing the FLAP gene. (a) The linkage scan for female MI patients and the one LOD drop region that includes the FLAP gene; (b) Microsatellite association for all MI patients: single marker association (black dots) and two, three, four and five marker haplotype association (black, blue, green and red horizontal lines, respectively). The blue and the red arrows indicate the location of the most significant haplotype association across the FLAP gene in males and females, respectively. (c) The FLAP gene structure, with exons shown as colored cylinders, and the location of all the SNPs typed in the region (green vertical lines). The green vertical lines indicate the position of the microsatellites (shown in b) and SNPs (shown in c) used in the analysis.

FIG. 13 shows linkage scan using framework microsatellite markers on chromosome 13 for male patients with ischemic stroke or TIA (n=342 in 164 families at 6 meiosis). The LOD score is expressed on the y axis and the distance from the pter in Kosambi cM on the x axis.

FIG. 14 shows a pairwise linkage disequilibrium (LD) between SNPs in a 60 Kb region encompassing FLAP. The markers are plotted equidistantly. Two measures of LD are shown: D′ in the upper left triangle and P values in the lower right triangle. Colored lines indicate the positions of the exons of FLAP and the green stars indicate the location of the markers of the at-risk haplotype A4. Scales for the LD strength are provided for both measures to the right.

### DETAILED DESCRIPTION OF THE INVENTION

Extensive genealogical information has been combined with powerful gene sharing methods to map a gene on chromosome 13q12 that is associated with myocardial infarction. A genome wide search for susceptibility genes for MI, using a framework map of 1000 microsatellite markers, revealed a locus suggestive of linkage on 13q12. Sixty families with 159 female MI patients that clustered within and including 6 meiotic events were used in linkage analysis. At first, only female MI patients were used in the linkage analysis in an effort to enrich for patients with stronger genetic factors contributing to their risk for MI. The epidemiological study of a population-based sample of Icelandic MI patients had previously suggested that the genetic factors for MI might be stronger for females than males, as the relative risk for siblings of female MI patients was significantly higher than the relative risk for siblings of male probands (1.59 (CI 1.47-1.73) vs. 1.35 (CI 1.28-1.42)) (unpublished data). The highest LOD score (2.5) was found at marker D13S289. The LOD score results for the families remained the same after adding 14 microsatellite markers to the candidate region. The inclusion of the additional markers increased the information on sharing by descent from 0.7 to 0.8, around the markers that gave the highest LOD scores. This linkage analysis mapped a gene contributing to MI to chromosome 13q12.

The candidate MI locus on chromosome 13q12 was then finely mapped with microsatellite markers. Patients with myocardial infarction and controls were initially genotyped with microsatellite markers with an average spacing between markers of less than 100 Kb over the 12 Mb candidate region. Initial haplotype association analysis that included all genotyped microsatellite markers across the MI candidate locus, resulted in several extended haplotypes composed of 4 and 5 microsatellite markers that were significantly associated with female MI (see, e.g., Tables 4 and 5 below). A region common to all these extended haplotypes, is defined by markers DG13S166 and D13S1238. This region includes only one gene, the FLAP nucleic acid sequence. The two marker haplotype involving alleles 0 and −2 for markers DG13S166 and D13S1238, respectively, was found in excess in patients. Specific variants of the gene were then sought that were associated with MI.

In order to screen for SNPs in the FLAP gene, the whole gene was sequenced, both exons and introns. Initially, 9 SNPs identified within the gene were genotyped in patients and controls. Additional microsatellite markers close to or within the FLAP gene were also genotyped in all patients and controls. Five publicly known SNPs that are located within a 200 Kb distance 5′ to the FLAP gene were also genotyped in patients and controls. Haplotype association analysis in this case-control study including these additional markers showed several different variants of the same haplotype that were all significantly associated with female MI (see, e.g., Table 6). Table 7 shows two haplotypes that are representative of these female MI risk haplotypes which are referred to herein as the female MI “at risk” haplotypes. The relative risk for male MI patients that had the female MI-“at risk” haplotype was increased (see, e.g., Table 7), indicating that the female MI-“at risk” haplotype also increased the risk of having an MI in males. These results further strengthened the hypothesis that the FLAP gene was an MI susceptibility gene.

SNP Haplotype Association to MI, and Subsequently to Stroke and PAOD

In an effort to identify haplotypes involving only SNP markers that associate with MI, additional SNPs were identified by sequencing the FLAP gene and the region flanking the gene. Currently, a total of 45 SNPs in 1343 patients and 624 unrelated controls have been genotyped. Two correlated series of SNP haplotypes have been observed in excess in patients, denoted as A and B in Table 9. The length of the haplotypes varies between 33 and 69 Kb, and the haplotypes cover one or two blocks of linkage disequilibrium. Both series of haplotypes contain the common allele 2 of the SNP SG13S25. All haplotypes in the A series contain the SNP DG00AAHID, while all haplotypes in the B series contain the SNP DG00AAHII. In the B series, the haplotypes B4, B5, and B6 have a relative risk (RR) greater than 2 and with allelic frequencies above 10%. The haplotypes in the A series have slightly lower RR and lower p-values, but higher frequency (15-16%). The haplotypes in series B and A are strongly correlated, i.e., the haplotypes in B define a subset of the haplotypes in A. Hence, haplotypes in series B are more specific than A. However, haplotypes in series A are more sensitive, i.e. they capture more individuals with the putative mutation, as is observed in the population attributable risk which is less for B than for A. Furthermore, these haplotypes show similar risk ratios and allelic frequencies for early-onset patients (defined as onset of first MI before the age of 55) and for both genders. In addition, analyzing various groups of patients with known risk factors, such as hypertension, high cholesterol, smoking and diabetes, do not reveal any significant correlation with these haplotypes, suggesting that the haplotypes in the FLAP gene represent an independent genetic susceptibility factor for MI.

Because stroke and PAOD are diseases that are closely related to MI (all occur on the basis of atherosclerosis the SNP haplotype in the FLAP gene that confers risk to MI was assessed to determine whether it also conferred risk of stroke and/or PAOD. Table 14 shows that haplotype A4 increases the risk of having a stroke to a similar extent as it increases the risk of having an MI. Although not as significantly, haplotype A4 also confers risk of developing PAOD.

The FLAP nucleic acid encodes a 5-lipoxygenase activating protein, which, in combination with 5-lipoxygenase (5-LO), is required for leukotriene synthesis. FLAP acts coordinately with 5-LO to catalyze the first step in the synthesis of leukotrienes from arachidonic acid. It catalyzes the conversion of arachidonic acid to 5(S)-hydroperoxy-6-trans-8,11,14-cis-eicosatetraenoic acid (5-HPETE), and further to the allylic epoxide 5 (S)-trans7,9 trans 11,14-cis-eicosatetraenoic acid (leukotriene A4, LTA4).

The leukotrienes are a family of highly potent biological mediators of inflammatory processes produced primarily by bone marrow derived leukocytes such as monocytes, macrophages, and neurophils. Both FLAP and 5-LO are detected within atherosclerosis lesions, indicating that the vessel itself can be a source of leukotrienes. It is demonstrated herein that the MI-risk FLAP haplotype is associated with higher serum leukotriene levels. Increased production of leukotriene in individuals with pre-existing atherosclerosis lesions may lead to plaque instability or friability of the fibrous cap leading to local thrombotic events. If this occurs in coronary artery arteries it leads to MI or unstable angina. If it occurs in the cerebrovasculature it leads to stroke or transient ischemic attack. If it occurs in large arteries to the limbs, it causes or exacerbates limb ischemia in persons with peripheral arterial occlusive disease (PAOD). Therefore, those with genetically influenced predisposition to produce higher leukotriene levels have higher risk for events due to pre-existing atherosclerosis such as MI.

Inhibitors of FLAP function impede translocation of 5-LO from the cytoplasm to the cell membrane and inhibit activation of 5-LO and thereby decrease leukotriene synthesis.

As a result of these discoveries, methods are now available for the treatment of myocardial infarction (MI) and acute coronary syndrome (ACS), as well as stroke and PAOD, through the use of leukotriene inhibitors, such as agents that inhibit leukotriene biosynthesis or antagonize signaling through leukotriene receptors. The term, “treatment” as used herein, refers not only to ameliorating symptoms associated with the disease or condition, but also preventing or delaying the onset of the disease or condition; preventing or delaying the occurrence of a second episode of the disease or condition; and/or also lessening the severity or frequency of symptoms of the disease or condition. In the case of atherosclerosis, “treatment” also refers to a minimization or reversal of the development of plaques. Methods are additionally available for assessing an individual's risk for MI, ACS, stroke or PAOD. In a preferred embodiment, the individual to be treated is an individual who is susceptible (at increased risk) for MI, ACS, stroke or PAOD, such as an individual who is in one of the representative target populations described herein.

Representative Target Populations

In one embodiment of the invention, an individual who is at risk for MI, ACS, stroke or PAOD is an individual who has an at-risk haplotype in FLAP, as described herein. In one embodiment, a haplotype associated with a susceptibility to myocardial infarction, ACS, stroke or PAOD comprises markers DG00AAFIU, SG13S25, DG00AAJFF, DG00AAHII, SG13S32 and SG13S35 at the 13q12 locus. In another embodiment, a haplotype associated with a susceptibility to myocardial infarction, ACS, stroke or PAOD comprises markers DG00AAFIU, SG13S25, DG00AAHII, SG13S30 and SG13S42 at the 13q12 locus. In a third embodiment, a haplotype associated with a susceptibility to myocardial infarction, ACS, stroke or PAOD comprises markers SG13S25, DG00AAHII, SG13S30 and SG13S42 at the 13q12 locus. In a fourth embodiment, a haplotype associated with a susceptibility to myocardial infarction, ACS, stroke or PAOD comprises markers DG00AAFIU, SG13S25, DG00AAHID, B_SNP_{—}310657 and SG13S32 at the 13q12 locus. In a fifth embodiment, a haplotype associated with a susceptibility to myocardial infarction, ACS, stroke or PAOD comprises markers SG13S25, DG00AAHID, B_SNP_{—}310657 and SG13S32 at the 13q12 locus. Additional haplotypes associated with a susceptibility to myocardial infarction, ACS, stroke or PAOD include the haplotypes shown in Tables 4, 5, 6, 7, 11, 12, and 19, as well as haplotypes comprising markers shown in Table 3.

Increased risk for MI, ACS, stroke or PAOD in individuals with a FLAP at-risk haplotype is logically conferred by increased production of leukotrienes in the arterial vessel wall or in bone-marrow derived inflammatory cells within the blood and/or arterial vessel wall. It is shown herein that FLAP at-risk haplotypes are associated with high serum leukotriene E4 levels. It is also shown herein that FLAP at-risk haplotypes are associated with higher production of LTB4 ex vivo. It is further shown herein that serum leukotriene levels (specifically, leukotrieneE4) correlate with serum CRP levels in myocardial infarction patients. Therefore, FLAP genetic variation drives high leukotriene levels (within the blood vessel and/or systemically) which in turn drive higher CRP levels which has been shown as a risk factor for MI. Accordingly, individuals with a FLAP at-risk haplotype are likely to have elevated serum CRP as well as other serum inflammatory markers. The level of serum CRP or other serum inflammatory markers can be used as a surrogate for the level of arterial wall inflammation initiated by lipid deposition and atherogenesis conferred by the presence of the at-risk FLAP haplotype.

In another embodiment of the invention, an individual who is at risk for MI, ACS, stroke or PAOD is an individual who has a polymorphism in a FLAP gene, in which the presence of the polymorphism is indicative of a susceptibility to MI, ACS, stroke or PAOD. The term “gene,” as used herein, refers to not only the sequence of nucleic acids encoding a polypeptide, but also the promoter regions, transcription enhancement elements, splice donor/acceptor sites, and other non-transcribed nucleic acid elements. Representative polymorphisms include those presented in Table 3, below.

In a further embodiment of the invention, an individual who is at risk for MI, ACS, stroke or PAOD is an individual who has an at-risk polymorphism in the 5-LO gene in the promoter region, as described herein.

In a fourth embodiment, an individual who is at risk for MI, ACS, stroke or PAOD is an individual who has an elevated inflammatory marker. An “elevated inflammatory marker,” as used herein, is the presence of an amount of an inflammatory marker that is greater, by an amount that is statistically significant, than the amount that is typically found in control individual(s) or by comparison of disease risk in a population associated with the lowest band of measurement (e.g., below the mean or median, the lowest quartile or the lowest quintile) compared to higher bands of measurement (e.g., above the mean or median, the second, third or fourth quartile; the second, third, fourth or fifth quintile). An “inflammatory marker” refers to a molecule that is indicative of the presence of inflammation in an individual, for example, C-reactive protein (CRP), serum amyloid A, fibrinogen, leukotriene levels (e.g., leukotriene E4), leukotriene metabolites (e.g., cysteinyl leukotriene 1), interleukin-6, tissue necrosis factor-alpha, soluble vasculare cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, myeloperoxidase (MPO), N-tyrosine) or other markers (see, e.g., Doggen, C. J. M. et al., J. Internal Med., 248:406-414 (2000); Ridker, P. M. et al., New Englnd. J. Med. 1997: 336: 973-979, Rettersol, L. et al., 2002: 160:433-440; Ridker, P. M. et. al., New England. J. Med., 2002: 347:1557-1565; Bermudez, E. A. et. al., Arterioscler. Thromb. Vasc. Biol., 2002: 22:1668-1673). In certain embodiments, the presence of such inflammatory markers can be measured in serum or urine.

In a fifth embodiment, an individual who is at risk for MI, ACS, stroke or PAOD is an individual who has increased LDL cholesterol and/or decreased HDL cholesterol levels. For example, the American Heart Association indicates that an LDL cholesterol level of less than 100 mg/dL is optimal; from 100-129 mg/dL is near/above optimal; from 130-159 mg/dL is borderline high; from 160-189 is high; and from 190 and up is very high. Therefore, an individual who is at risk for MI, ACS, stroke or PAOD because of an increased LDL cholesterol level is, for example, an individual who has more than 100 mg/dL cholesterol, such as an individual who has a near/above optimal level, a borderline high level, a high level or a very high level. Similarly, the American Heart Association indicates that an HDL cholesterol level of less than 40 mg/dL is a major risk factor for heart disease; and an HDL cholesterol level of 60 mg/dL or more is protective against heart disease. Thus, an individual who is at risk for MI, ACS, stroke or PAOD because of a decreased HDL cholesterol level is, for example, an individual who has less than 60 mg/dL HDL cholesterol, such as an individual who has less than 40 mg/dL HDL cholesterol.

In a sixth embodiment, an individual who is at risk for MI, ACS, stroke or PAOD is an individual who has increased leukotriene synthesis. “Increased leukotriene synthesis,” as used herein, indicates an amount of production of leukotrienes that is greater, by an amount that is statistically significant, than the amount of production of leukotrienes that is typically found in control individual(s) or by comparison of leukotriene production in a population associated with the lowest band of measurement (e.g., below the mean or median, the lowest quartile or the lowest quintile) compared to higher bands of measurement (e.g., above the mean or median, the second, third or fourth quartile; the second, third, fourth or fifth quintile). For example, the FLAP at-risk haplotypes correlate with increased serum leukotriene synthesis levels, and with increased production of leukotrienes ex vivo. An individual can be assessed for the presence of increased leukotriene synthesis by a variety of methods. For example, an individual can be assessed for an increased risk of MI, ACS, stroke, PAOD or atherosclerosis, by assessing the level of a leukotriene metabolite (e.g., LTE4) in a sample (e.g., serum, plasma or urine) from the individual. Samples containing blood, cells, or tissue can also be obtained from an individual and used to assess leukotriene or leukotriene metabolite production ex vivo under appropriate assay conditions. An increased level of leukotriene metabolites, and/or an increased level of leukotriene production ex vivo, is indicative of increased production of leukotrienes in the individual, and of an increased risk of MI, ACS, stroke, PAOD or atherosclerosis.

In a further embodiment, an individual who is at risk for MI, ACS, or stroke is an individual who has already experienced at least one MI, ACS event or stroke, or who has stable angina, and is therefore at risk for a second MI, ACS event or stroke. In another embodiment, an individual who is at risk for MI, ACS, stroke or PAOD is an individual who has atherosclerosis or who requires treatment (e.g., angioplasty, stents, revascularization procedure) to restore blood flow in arteries.

In further embodiments, an individual who is at risk for MI, stroke or PAOD is an individual having asymptomatic ankle/brachial index of less than 0.9; an individual who is at risk for stroke, is an individual who has had one or more transient ischemic attacks; who has had transient monocular blindness; has had a carotid endareterectomy; or has asymptomatic carotid stenosis; an individual who is at risk for PAOD, is an individual who has (or had) claudication, limb ischemia leading to gangrene, ulceration or amputation, or has had a revascularization procedure.

In additional embodiments, an individual who is at risk for MI, ACS, stroke or PAOD is an individual who has diabetes; hypertension; hypercholesterolemia; elevated triglycerides (e.g., >200 mg/dl); elevated lp(a); obesity; ankle/brachial index (ABI) less than 0.9; and/or is a past or current smoker.

Individuals at risk for MI, ACS, stroke or PAOD may fall into more than one of these representative target populations. For example, an individual may have experienced at least one MI, ACS event, transient ischemic attack, transient monocular blindness, or stroke, and may also have an increased level of an inflammatory marker. As used therein, the term “individual in a target population” refers to an individual who is at risk for MI, ACS, stroke or PAOD who falls into at least one of the representative target populations described above.

Assessment for At-Risk Haplotypes

A “haplotype,” as described herein, refers to a combination of genetic markers (“alleles”), such as those set forth in Table 3. In a certain embodiment, the haplotype can comprise one or more alleles, two or more alleles, three or more alleles, four or more alleles, or five or more alleles. The genetic markers are particular “alleles” at “polymorphic sites” associated with FLAP. A nucleotide position at which more than one sequence is possible in a population (either a natural population or a synthetic population, e.g., a library of synthetic molecules), is referred to herein as a “polymorphic site”. Where a polymorphic site is a single nucleotide in length, the site is referred to as a single nucleotide polymorphism (“SNP”). For example, if at a particular chromosomal location, one member of a population has an adenine and another member of the population has a thymine at the same position, then this position is a polymorphic site, and, more specifically, the polymorphic site is a SNP. Polymorphic sites can allow for differences in sequences based on substitutions, insertions or deletions. Each version of the sequence with respect to the polymorphic site is referred to herein as an “allele” of the polymorphic site. Thus, in the previous example, the SNP allows for both an adenine allele and a thymine allele.

Typically, a reference sequence is referred to for a particular sequence. Alleles that differ from the reference are referred to as “variant” alleles. For example, the reference FLAP sequence is described herein by SEQ ID NO: 1. The term, “variant FLAP”, as used herein, refers to a sequence that differs from SEQ ID NO: 1, but is otherwise substantially similar. The genetic markers that make up the haplotypes described herein are FLAP variants.

Additional variants can include changes that affect a polypeptide, e.g., the FLAP polypeptide. These sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by a FLAP nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a susceptibility to MI, ACS, stroke or PAOD can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of the polypeptide. The polypeptide encoded by the reference nucleotide sequence is the “reference” polypeptide with a particular reference amino acid sequence, and polypeptides encoded by variant alleles are referred to as “variant” polypeptides with variant amino acid sequences.

Haplotypes are a combination of genetic markers, e.g., particular alleles at polymorphic sites. The haplotypes described herein, e.g., having markers such as those shown in Table 3, are found more frequently in individuals with MI, ACS, stroke or PAOD than in individuals without MI, ACS, stroke or PAOD. Therefore, these haplotypes have predictive value for detecting a susceptibility to MI, ACS, stroke or PAOD in an individual. The haplotypes described herein are in some cases a combination of various genetic markers, e.g., SNPs and microsatellites. Therefore, detecting haplotypes can be accomplished by methods known in the art for detecting sequences at polymorphic sites, such as the methods described above.

In certain methods described herein, an individual who is at risk for MI, ACS, stroke or PAOD is an individual in whom an at-risk haplotype is identified. In one embodiment, the at-risk haplotype is one that confers a significant risk of MI, ACS, stroke or PAOD. In one embodiment, significance associated with a haplotype is measured by an odds ratio. In a further embodiment, the significance is measured by a percentage. In one embodiment, a significant risk is measured as an odds ratio of at least about 1.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95%, and 98%. In a further embodiment, a significant increase in risk is at least about 50%. It is understood however, that identifying whether a risk is medically significant may also depend on a variety of factors, including the specific disease, the haplotype, and often, environmental factors.

An at-risk haplotype in, or comprising portions of, the FLAP gene, in one where the haplotype is more frequently present in an individual at risk for MI, ACS, stroke or PAOD (affected), compared to the frequency of its presence in a healthy individual (control), and wherein the presence of the haplotype is indicative of susceptibility to MI, ACS, stroke or PAOD. As an example of a simple test for correlation would be a Fisher-exact test on a two by two table. Given a cohort of chromosomes the two by two table is constructed out of the number of chromosomes that include both of the haplotypes, one of the haplotype but not the other and neither of the haplotypes.

In certain embodiments at-risk haplotype is an at-risk haplotype within or near FLAP that significantly correlates with a haplotype such as a halotype shown in Table 4; a haplotype shown in Table 5; a haplotype shown in Table 13; haplotype B4; haplotype B5; haplotype B6; haplotype A4; haplotype A5; or haplotype HapB. In other embodiments, an at-risk haplotype comprises an at-risk haplotype within or near FLAP that significantly correlates with susceptibility to myocardial infarction or stroke. In a particular embodiment, a haplotype associated with a susceptibility to myocardial infarction, ACS, stroke or PAOD comprises markers DG00AAFIU, SG13S25, DG00AAJFF, DG00AAHII, SG13S32 and SG13S35 at the 13q12 locus. In another embodiment, a haplotype associated with a susceptibility to myocardial infarction, ACS, stroke or PAOD comprises markers DG00AAFIU, SG13S25, DG00AAHII, SG13S30 and SG13S42 at the 13q12 locus. In a third embodiment, a haplotype associated with a susceptibility to myocardial infarction, ACS, stroke or PAOD comprises markers SG13S25, DG00AAHII, SG13S30 and SG13S42 at the 13q12 locus. In a fourth embodiment, a haplotype associated with a susceptibility to myocardial infarction, ACS, stroke or PAOD comprises markers DG00AAFIU, SG13S25, DG00AAHID, B_SNP_{—}310657 and SG13S32 at the 13q12 locus. In other embodiments, the at-risk haplotype is selected from the group consisting of: haplotype B4, B5, B6, A4 and A5. The at-risk haplotype can also comprise a combination of the markers in the haplotypes B4, B5, B6, A4 and/or A5. In further embodiments, the at-risk haplotype can be haplotype HapB. In other embodiments, the at-risk haplotype comprises a polymorphism shown in Table 3.

Standard techniques for genotyping for the presence of SNPs and/or microsatellite markers can be used, such as fluorescent based techniques (Chen, et al., Genome Res. 9, 492 (1999)), PCR, LCR, Nested PCR and other techniques for nucleic acid amplification. In a preferred embodiment, the method comprises assessing in an individual the presence or frequency of SNPs and/or microsatellites in, comprising portions of, the FLAP gene, wherein an excess or higher frequency of the SNPs and/or microsatellites compared to a healthy control individual is indicative that the individual is susceptible to MI, ACS, stroke or PAOD. See, for example, Table 3 (below) for SNPs and markers that can form haplotypes that can be used as screening tools. These markers and SNPs can be identified in at-risk haploptypes. For example, an at-risk haplotype can include microsatellite markers and/or SNPs such as those set forth in Table 3. The presence of the haplotype is indicative of a susceptibility to MI, ACS, stroke or PAOD, and therefore is indicative of an individual who falls within a target population for the treatment methods described herein.

Haplotype analysis involves defining a candidate susceptibility locus using LOD scores. The defined regions are then ultra-fine mapped with microsatellite markers with an average spacing between markers of less than 100 Kb. All usable microsatellite markers that found in public databases and mapped within that region can be used. In addition, microsatellite markers identified within the deCODE genetics sequence assembly of the human genome can be used. The frequencies of haplotypes in the patient and the control groups using an expectation-maximization algorithm can be estimated (Dempster A. et al., 1977. J. R. Stat. Soc. B, 39:1-389). An implementation of this algorithm that can handle missing genotypes and uncertainty with the phase can be used. Under the null hypothesis, the patients and the controls are assumed to have identical frequencies. Using a likelihood approach, an alternative hypothesis where a candidate at-risk-haplotype, which can include the markers described herein, is allowed to have a higher frequency in patients than controls, while the ratios of the frequencies of other haplotypes are assumed to be the same in both groups is tested. Likelihoods are maximized separately under both hypotheses and a corresponding 1-df likelihood ratio statistic is used to evaluate the statistic significance.

To look for at-risk-haplotypes in the 1-lod drop, for example, association of all possible combinations of genotyped markers is studied, provided those markers span a practical region. The combined patient and control groups can be randomly divided into two sets, equal in size to the original group of patients and controls. The haplotype analysis is then repeated and the most significant p-value registered is determined. This randomization scheme can be repeated, for example, over 100 times to construct an empirical distribution of p-values. In a preferred embodiment, a p-value of <0.05 is indicative of an at-risk haplotype.

A detailed discussion of haplotype analysis follows.

Haplotype Analysis

Our general approach to haplotype analysis involves using likelihood-based inference applied to NEsted MOdels. The method is implemented in our program NEMO, which allows for many polymorphic markers, SNPs and microsatellites. The method and software are specifically designed for case-control studies where the purpose is to identify haplotype groups that confer different risks. It is also a tool for studying LD structures.

When investigating haplotypes constructed from many markers, apart from looking at each haplotype individually, meaningful summaries often require putting haplotypes into groups. A particular partition of the haplotype space is a model that assumes haplotypes within a group have the same risk, while haplotypes in different groups can have different risks. Two models/partitions are nested when one, the alternative model, is a finer partition compared to the other, the null model, i.e, the alternative model allows some haplotypes assumed to have the same risk in the null model to have different risks. The models are nested in the classical sense that the null model is a special case of the alternative model. Hence traditional generalized likelihood ratio tests can be used to test the null model against the alternative model. Note that, with a multiplicative model, if haplotypes hᵢ and hⱼ are assumed to have the same risk, it corresponds to assuming that fᵢ/pᵢ=fⱼ/pⱼ where f and p denote haplotype frequencies in the affected population and the control population respectively.

One common way to handle uncertainty in phase and missing genotypes is a two-step method of first estimating haplotype counts and then treating the estimated counts as the exact counts, a method that can sometimes be problematic (e.g., see the information measure section below) and may require randomization to properly evaluate statistical significance. In NEMO, maximum likelihood estimates, likelihood ratios and p-values are calculated directly, with the aid of the EM algorithm, for the observed data treating it as a missing-data problem.

NEMO allows complete flexibility for partitions. For example, the first haplotype problem described in the Methods section on Statistical analysis considers testing whether h₁ has the same risk as the other haplotypes h₂, . . . , hₖ. Here the alternative grouping is [h₁], [h₂, . . . , hₖ] and the null grouping is [h₁, . . . , hₖ]. The second haplotype problem in the same section involves three haplotypes h₁=G0, h₂=GX and h₃=AX, and the focus is on comparing h₁ and h₂. The alternative grouping is [h₁], [h₂], [h₃] and the null grouping is [h₁, h₂], [h₃]. If composite alleles exist, one could collapse these alleles into one at the data processing stage, and performed the test as described. This is a perfectly valid approach, and indeed, whether we collapse or not makes no difference if there were no missing information regarding phase. But, with the actual data, if each of the alleles making up a composite correlates differently with the SNP alleles, this will provide some partial information on phase. Collapsing at the data processing stage will unnecessarily increase the amount of missing information. A nested-models/partition framework can be used in this scenario. Let h₂ be split into h₂ₐ, h_{2b}, . . . , h₂ₑ, and h₃ be split into h₃ₐ, h_{3b}, . . . , h₃ₑ. Then the alternative grouping is [h₁], [h₂ₐ, h_{2b}, . . . , h₂ₑ], [h₃ₐ, h_{3b}, . . . , h₃ₑ] and the null grouping is [h₁, h₂ₐ, h_{2b}, . . . , h₂ₑ], [h₃ₐ, h_{3b}, . . . , h₃ₑ]. The same method can be used to handle composite where collapsing at the data processing stage is not even an option since L_{C} represents multiple haplotypes constructed from multiple SNPs. Alternatively, a 3-way test with the alternative grouping of [h₁], [h₂ₐ, h_{2b}, . . . , h₂ₑ], [h₃ₐ, h_{3b}, . . . , h₃ₑ] versus the null grouping of [h₁, h₂ₐ, h_{2b}, . . . , h₂ₑ, h₃ₐ, h_{3b}, . . . , h₃ₑ] could also be performed. Note that the generalized likelihood ratio test-statistic would have two degrees of freedom instead of one.

Measuring Information

Even though likelihood ratio tests based on likelihoods computed directly for the observed data, which have captured the information loss due to uncertainty in phase and missing genotypes, can be relied on to give valid p-values, it would still be of interest to know how much information had been lost due to the information being incomplete. Interestingly, one can measure information loss by considering a two-step procedure to evaluating statistical significance that appears natural but happens to be systematically anti-conservative. Suppose we calculate the maximum likelihood estimates for the population haplotype frequencies calculated under the alternative hypothesis that there are differences between the affected population and control population, and use these frequency estimates as estimates of the observed frequencies of haplotype counts in the affected sample and in the control sample. Suppose we then perform a likelihood ratio test treating these estimated haplotype counts as though they are the actual counts. We could also perform a Fisher's exact test, but we would then need to round off these estimated counts since they are in general non-integers. This test will in general be anti-conservative because treating the estimated counts as if they were exact counts ignores the uncertainty with the counts, overestimates the effective sample size and underestimates the sampling variation. It means that the chi-square likelihood-ratio test statistic calculated this way, denoted by Λ*, will in general be bigger than Λ, the likelihood-ratio test-statistic calculated directly from the observed data as described in methods. But Λ* is useful because the ratio Λ/Λ* happens to be a good measure of information, or 1−(Λ/Λ*) is a measure of the fraction of information lost due to missing information. This information measure for haplotype analysis is described in Nicolae and Kong, Technical Report 537, Department of Statistics, University of Statistics, University of Chicago, Revised for Biometrics (2003) as a natural extension of information measures defined for linkage analysis, and is implemented in NEMO.

Statistical Analysis.

For single marker association to the disease, the Fisher exact test can be used to calculate two-sided p-values for each individual allele. All p-values are presented unadjusted for multiple comparisons unless specifically indicated. The presented frequencies (for microsatellites, SNPs and haplotypes) are allelic frequencies as opposed to carrier frequencies. To minimize any bias due the relatedness of the patients who were recruited as families for the linkage analysis, first and second-degree relatives can be eliminated from the patient list. Furthermore, the test can be repeated for association correcting for any remaining relatedness among the patients, by extending a variance adjustment procedure described in Risch, N. & Teng, J. (Genome Res., 8:1278-1288 (1998)). The relative power of family-based and case-control designs for linkage disequilibrium studies of complex human diseases I. DNA pooling. (ibid) for sibships so that it can be applied to general familial relationships, and present both adjusted and unadjusted p-values for comparison. The differences are in general very small as expected. To assess the significance of single-marker association corrected for multiple testing we carried out a randomisation test using the same genotype data. Cohorts of patients and controls can be randomized and the association analysis redone multiple times (e.g., up to 500,000 times) and the p-value is the fraction of replications that produced a p-value for some marker allele that is lower than or equal to the p-value we observed using the original patient and control cohorts.

For both single-marker and haplotype analyses, relative risk (RR) and the population attributable risk (PAR) can be calculated assuming a multiplicative model (haplotype relative risk model), (Terwilliger, J. D. & Ott, J., Hum Hered, 42, 337-46 (1992) and Falk, C. T. & Rubinstein, P, Ann Hum Genet 51 (Pt 3), 227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes hᵢ and hⱼ, risk(hᵢ)/risk(hⱼ)=(fᵢ/pᵢ)/(fⱼ/pⱼ), where f and p denote respectively frequencies in the affected population and in the control population. While there is some power loss if the true model is not multiplicative, the loss tends to be mild except for extreme cases. Most importantly, p-values are always valid since they are computed with respect to null hypothesis.

In general, haplotype frequencies are estimated by maximum likelihood and tests of differences between cases and controls are performed using a generalized likelihood ratio test (Rice, J. A. Mathematical Statistics and Data Analysis, 602 (International Thomson Publishing, (1995)). deCODE's haplotype analysis program called NEMO, which stands for NEsted MOdels, can be used to calculate all the haplotype results. To handle uncertainties with phase and missing genotypes, it is emphasized that we do not use a common two-step approach to association tests, where haplotype counts are first estimated, possibly with the use of the EM algorithm, Dempster, (A. P., Laird, N. M. & Rubin, D. B., Journal of the Royal Statistical Society B, 39, 1-38 (1971)) and then tests are performed treating the estimated counts as though they are true counts, a method that can sometimes be problematic and may require randomisation to properly evaluate statistical significance. Instead, with NEMO, maximum likelihood estimates, likelihood ratios and p-values are computed with the aid of the EM-algorithm directly for the observed data, and hence the loss of information due to uncertainty with phase and missing genotypes is automatically captured by the likelihood ratios. Even so, it is of interest to know how much information is retained, or lost, due to incomplete information. Described herein is such a measure that is natural under the likelihood framework. For a fixed set of markers, the simplest tests performed compare one selected haplotype against all the others. Call the selected haplotype h₁ and the others h₂, . . . , hₖ. Let p₁, . . . , pₖ denote the population frequencies of the haplotypes in the controls, and f₁, . . . , fₖ denote the population frequencies of the haplotypes in the affecteds. Under the null hypothesis, fᵢ=pᵢ for all i. The alternative model we use for the test assumes h₂, . . . , hₖ to have the same risk while h₁ is allowed to have a different risk. This implies that while p₁ can be different from f₁, fᵢ/(f₂+ . . . +fₖ)=pᵢ/(p2+ . . . +pₖ)=βᵢ for i=2, . . . , k. Denoting f₁/p₁ by r, and noting that β₂+ . . . +βₖ=1, the test statistic based on generalized likelihood ratios is
Λ=2[l(\hat{r}, \hat{p}₁, \hat{β}₂, . . . , \hat{β}ₖ₋₁)−l(1, \tilde{p}₁, \tilde{β}₂, . . . , \tilde{β}ₖ₋₁)]
where l denotes logₑlikelihood and ~ and ˆ denote maximum likelihood estimates under the null hypothesis and alternative hypothesis respectively. Λ has asymptotically a chi-square distribution with 1-df, under the null hypothesis. Slightly more complicated null and alternative hypotheses can also be used. For example, let h₁ be G0, h₂ be GX and h₃ be AX. When comparing G0 against GX, i.e., this is the test which gives estimated RR of 1.46 and p-value=0.0002, the null assumes G0 and GX have the same risk but AX is allowed to have a different risk. The alternative hypothesis allows, for example, three haplotype groups to have different risks. This implies that, under the null hypothesis, there is a constraint that f₁/p₁=f₂/p₂, or w=[f₁/p₁]/[f₂/p₂]=1. The test statistic based on generalized likelihood ratios is
Λ=2[l(\hat{p}₁,\hat{f}₁,\hat{p}₂,ŵ)−l(\tilde{p}₁, \tilde{f}₁,\tilde{p}₂, 1)]
that again has asymptotically a chi-square distribution with 1-df under the null hypothesis. If there are composite haplotypes (for example, h₂ and h₃), that is handled in a natural manner under the nested models framework.

LD between pairs of SNPs can be calculated using the standard definition of D′ and R² (Lewontin, R., Genetics 49, 49-67 (1964) and Hill, W. G. & Robertson, A. Theor. Appl. Genet. 22, 226-231 (1968)).Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combination to elucidate the LD structure in a particular region, we plot D′ in the upper left corner and the p-value in the lower right corner. In the LD plots the markers can be plotted equidistant rather than according to their physical location, if desired.

Statistical Methods for Linkage Analysis

Multipoint, affected-only allele-sharing methods can be used in the analyses to assess evidence for linkage. Results, both the LOD-score and the non-parametric linkage (NPL) score, can obtained using the program Allegro (Gudbjartsson et al., Nat. Genet. 25:12-3, 2000). Our baseline linkage analysis uses the Spairs scoring function (Whittemore, A. S., Halpern, J. (1994), Biometrics 50:118-27; Kruglyak L, et al. (1996), Am J Hum Genet 58:1347-63), the exponential allele-sharing model (Kong, A. and Cox, N. J. (1997), Am J Hum Genet 61:1179-88) and a family weighting scheme that is halfway, on the log-scale, between weighting each affected pair equally and weighting each family equally. The information measure we use is part of the Allegro program output and the information value equals zero if the marker genotypes are completely uninformative and equals one if the genotypes determine the exact amount of allele sharing by decent among the affected relatives (Gretarsdottir et al., Am. J. Hom. Genet, 70:593-603, (2002)). We computed the P-values two different ways and here report the less significant result. The first P-value can be computed on the basis of large sample theory; the distribution of Zᵢᵣ=√(2[logₑ(10)LOD]) approximates a standard normal variable under the null hypothesis of no linkage (Kong, A. and Cox, N. J. (1997), Am J Hum Genet 61:1179-88). The second P-value can be calculated by comparing the observed LOD-score with its complete data sampling distribution under the null hypothesis (e.g., Gudbjartsson et al., Nat. Genet. 25:12-3, 2000). When the data consist of more than a few families, these two P-values tend to be very similar.

Methods of Treatment

The present invention encompasses methods of treatment (prophylactic and/or therapeutic, as described above) for MI, ACS, stroke or PAOD in individuals, such as individuals in the target populations described above, as well as for other diseases and conditions associated with FLAP or with other members of the leukotriene pathway (e.g., for atherosclerosis). Members of the “leukotriene pathway,” as used herein, include polypeptides (e.g., enzymes, receptors) and other molecules that are associated with production of leukotrienes: for example, enzymes such as FLAP, 5-LO, other leukotriene biosynthetic enzymes (e.g., leukotriene C4 synthetase, leukotriene A4 hydrolase); receptors or binding agents of the enzymes; leukotrienes such as LTA4, LTB4, LTC4, LTD4, LTE4, Cys LT1, and Cys LT2; and receptors of leukotrienes (e.g., leukotriene B4 receptor 1 (BLT1), leukotriene B4 receptor 2 (BLT2), cysteinyl leukotriene receptor 1 (CysLTR1), cysteinyl leukotriene receptor 2 (CysLTR2)).

In particular, the invention relates to methods of treatment for myocardial infarction or susceptibility to myocardial infarction (for example, for individuals in an at-risk population such as those described above); as well as methods of treatment for acute coronary syndrome (e.g., unstable angina, non-ST-elevation myocardial infarction (NSTEMI) or ST-elevation myocardial infarction (STEMI)); methods for reducing risk of MI, stroke or PAOD in persons with asymptomatic ankle/brachial index less than 0.9; for decreasing risk of a second myocardial infarction; for stroke or susceptibility to stroke; for transient ischemic attack; for transient monocular blindness; for decreasing risk of a second stroke; for PAOD or susceptibility to PAOD; for ABI less than 0.9; for claudication or limb ischemia; for atherosclerosis, such as for patients requiring treatment (e.g., angioplasty, stents, revascularization procedure) to restore blood flow in arteries (e.g., coronary, carotid, and/or femoral arteries); for treatment of asymptomatic ankle/brachial index of less than 0.9; and/or for decreasing leukotriene synthesis (e.g., for treatment of MI, ACS, stroke or PAOD). The invention additionally pertains to use of one or more leukotriene synthesis inhibitors, as described herein, for the manufacture of a medicament for the treatment of MI, ACS, stroke, PAOD and/or atherosclerosis, e.g., using the methods described herein.

In the methods of the invention, a “leukotriene synthesis inhibitor” is used. In one embodiment, a “leukotriene synthesis inhibitor” is an agent that inhibits FLAP polypeptide activity and/or FLAP nucleic acid expression, as described herein (e.g., a nucleic acid antagonist). In another embodiment, a leukotriene synthesis inhibitor is an agent that inhibits polypeptide activity and/or nucleic acid expression of another member of the leukotriene biosynthetic pathway (e.g., 5-LO; LTC4S; LTA4H; LTB4DH). In still another embodiment, a leukotriene synthesis inhibitor is an agent that alters activity or metabolism of a leukotriene (e.g., an antagonist of a leukotriene; an antagonist of a leukotriene receptor). In preferred embodiments, the leukotriene synthesis inhibitor alters activity and/or nucleic acid expression of FLAP or of 5-LO, or alters interaction between FLAP and 5-LO.

Leukotriene synthesis inhibitors can alter polypeptide activity or nucleic acid expression of a member of the leukotriene pathway by a variety of means, such as, for example, by catalytically degrading, downregulating or interfering with the expression, transcription or translation of a nucleic acid encoding the member of the leukotriene pathway; by altering posttranslational processing of the polypeptide; by altering transcription of splicing variants; or by interfering with polypeptide activity (e.g., by binding to the polypeptide, or by binding to another polypeptide that interacts with that member of the leukotriene pathway, such as a FLAP binding agent as described herein or some other binding agent of a member of the leukotriene pathway; by altering interaction among two or more members of the leukotriene pathway (e.g., interaction between FLAP and 5-LO); or by antagonizing activity of a member of the leukotriene pathway.

Representative leukotriene synthesis inhibitors include the following:

agents that inhibit activity of a member of the leukotriene biosynthetic pathway (e.g., FLAP, 5-LO), LTC4S, LTA4H, LTB4DH, such as the agents presented in the Agent Table below; agents that inhibit activity of receptors of members of the leukotriene pathway, such as FLAP receptors, LTA4 receptors, LTB4 receptors, LTC4 receptors, LTD4 receptors, TLE4 receptors, Cys LT1 receptors, Cys LT2 receptors, 5-LO receptors; BLT1; BLT2; CysLTR1; CysLTR2; agents that bind to the members of the leukotriene pathway, such as FLAP binding agents (e.g., 5-LO), agents that bind to receptors of members of the leukotriene pathway (e.g., leukotriene receptor antagonists); or agents that bind to a leukotriene (e.g., to LTA4, LTB4, LTC4, LTD4, LTE4, Cys LT1, Cys LT2) or otherwise affect (e.g., increase or decrease) activity of the leukotriene;

antibodies to leukotrienes;

antisense nucleic acids or small double-stranded interfering RNA, to nucleic acids encoding FLAP, 5-LO, or a leukotriene synthetase or other member of the leukotriene pathway, or fragments or derivatives thereof, including antisense nucleic acids to nucleic acids encoding the FLAP, 5-LO or leukotriene synthetase polypeptides, and vectors comprising such antisense nucleic acids (e.g., nucleic acid, cDNA, and/or mRNA, double-stranded interfering RNA, or a nucleic acid encoding an active fragment or derivative thereof, or an oligonucleotide; for example, the complement of one of SEQ ID Nos. 1 or 3, or a nucleic acid complementary to the nucleic acid encoding SEQ ID NO: 2, or fragments or derivatives thereof);

peptidomimetics; fusion proteins or prodrugs thereof; ribozymes; other small molecules; and

other agents that alter (e.g., inhibit or antagonize) expression of a member of the leukotriene pathway, such as FLAP or 5-LO nucleic acid expression or polypeptide activity, or that regulate transcription of FLAP splicing variants or 5-LO splicing variants (e.g., agents that affect which splicing variants are expressed, or that affect the amount of each splicing variant that is expressed).

More than one leukotriene synthesis inhibitor can be used concurrently, if desired.

The therapy is designed to alter activity of a FLAP polypeptide, a 5-LO polypeptide, or another member of the leukotriene pathway in an individual, such as by inhibiting or antagonizing activity. For example, a leukotriene synthesis inhibitor can be administered in order to decrease synthesis of leukotrienes within the individual, or to downregulate or decrease the expression or availability of the FLAP nucleic acid or specific splicing variants of the FLAP nucleic acid. Downregulation or decreasing expression or availability of a native FLAP nucleic acid or of a particular splicing variant could minimize the expression or activity of a defective nucleic acid or the particular splicing variant and thereby minimize the impact of the defective nucleic acid or the particular splicing variant. Similarly, for example, a leukotriene synthesis inhibitor can be administered in order to downregulate or decrease the expression or availability of the nucleic acid encoding 5-LO or specific splicing variants of the nucleic acid encoding 5-LO.

The leukotriene synthesis inhibitor(s) are administered in a therapeutically effective amount (i.e., an amount that is sufficient to treat the disease or condition, such as by ameliorating symptoms associated with the disease or condition, preventing or delaying the onset of the disease or condition, and/or also lessening the severity or frequency of symptoms of the disease or condition). The amount which will be therapeutically effective in the treatment of a particular individual's disease or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

In preferred embodiments of the invention, the leukotriene synthesis inhibitor agent is an agent that inhibits activity of FLAP and/or of 5-LO. Preferred agents include the following, as set forth in the Agent Table:

| | Product Name | |
|---|---|---|
| Company | (Code) | Structure |
| Abbot | atreleuton (ABT-761) | 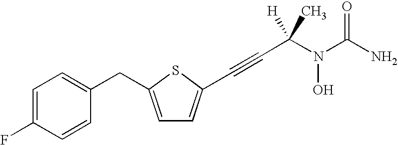 |
| Abbott | A-81834 | 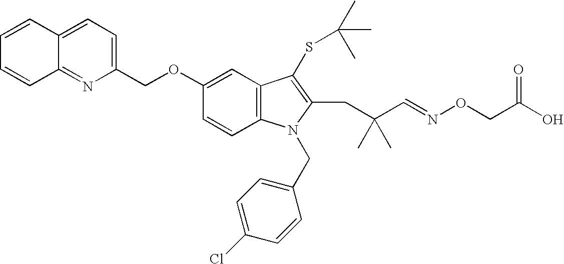 |
| Abbott | A-86886 | 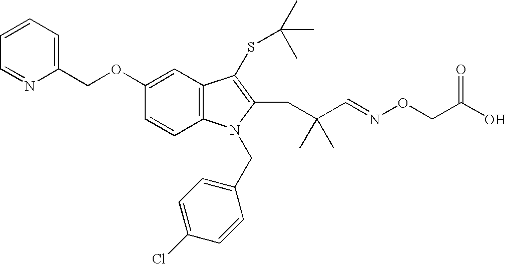 |
| Abbott | A-93178 | 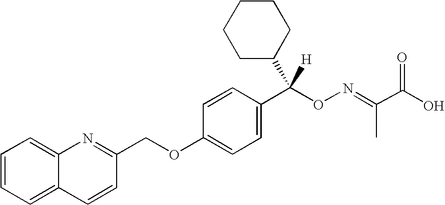 |
| AstraZeneca | AZD-4407 | 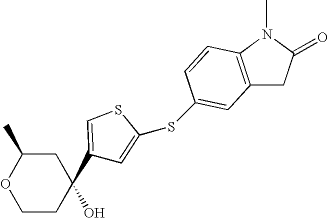 |
| AstraZeneca | ZD-2138 | 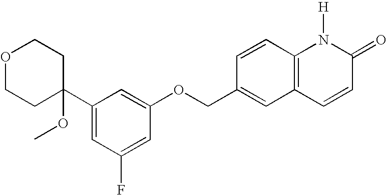 |
| Bayer | BAY-X-1005 | 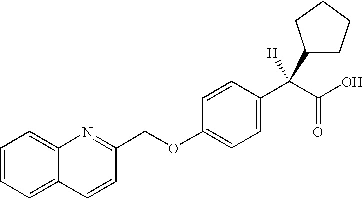 |
| Merck | MK-0591 | 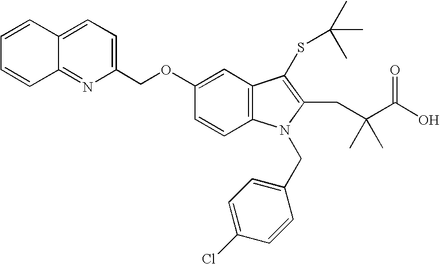 |
| Merck | MK-866 | |
| Merck | MK-886 | 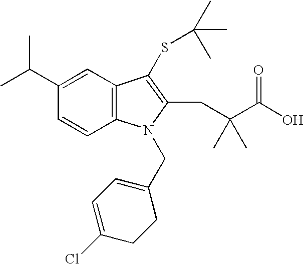 |
| Pfizer | CJ-13610 | |

| | | | Date Patent Issued/ | |
|---|---|---|---|---|
| Company | Chemical Name | Patent Ref | Application Published | MOA |
| Abbott | (R)-(+)-N-[3(5-[(4- | U.S. Pat. No. 5288751, | Feb. 22, 1994 | 5-LPO inhibitor |
| | fluorophenyl)methyl]-2thienyl]- | U.S. Pat. No. 5288743, | Apr. 1, 1997 | |
| | 1methyl-2-propynyl]-N- | U.S. Pat. No. 5616596 | | |
| | hydroxurea | | | |
| Abbott | 3-(3-(1,1-dimethylethylthio-5- | WO9203132, | Mar. 5, 1992, | FLAP inhibitor |
| | (quinoline-2-ylmethoxy)-1-(4- | U.S. Pat. No. 5459150 | Oct. 17, 1995 | |
| | chloromethylphenyl)indole-2-yl)- | | | |
| | 2,2-dimethylpropionaldehyde | | | |
| | oxime-0-2-acetic acid | | | |
| Abbott | 3-(3-(1,1-dimethylethylthio-5- | WO9203132, | Mar. 5, 1992, | 5-LPO inhibitor |
| | (pyridin-2-ylmethoxy)-1-(4- | U.S. Pat. No. 5459150 | Oct. 17, 1995 | |
| | chloromethylphenyl)indole-2-yl)- | | | |
| | 2,2-dimethylpropionaldehyde | | | |
| | oxime-0-2-acetic acid | | | |
| Abbott | | | | FLAP inhibitor |
| AstraZeneca | | EP623614 | Sep. 11, 1994 | 5-LPO inhibitor |
| AstraZeneca | 6-((3-fluoro-5- | EP466452 | | 5-LPO inhibitor |
| | (tetrahydro-4-methoxy-2H- | | | |
| | pyran- | | | |
| | 4yl)phenoxy)methyl)-1- | | | |
| | methyl-2(1H)- | | | |
| | quinlolinone | | | |
| | (alternatively NH can | | | |
| | be N-methyl) | | | |
| Bayer | (R)-(+)-alpha- | U.S. Pat. No. 5970215 | | FLAP inhibitor |
| | cyclopentyl-4-(2- | EP 344519, | | |
| | quinolinylmethoxyl)- | DE 19880531 | | |
| | Benzeneacetic acid | | | |
| Merck | 1-((4- | EP 419049, | | FLAP inhibitor |
| | chlorophenyl)methyl)-3- | U.S. Pat. No. 19890822 | | |
| | ((1,1- | | | |
| | dimethylethyl)thio)- | | | |
| | alpha, alpha-dimethyl-5- | | | |
| | (2-quinolinylmethoxy)-1H- | | | |
| | Indole-2-propanoic acid | | | |
| Merck | (3[3-)4-chlorobenzyl)-3-t-butyl- | | | 5-LPO inhibitor |
| | thio-5-isopropylindol-2yl]2,2- | | | |
| | dimethyl-proanoic acid | | | |
| Merck | 1-((4- | EP 419049, | | 5-LPO inhibitor |
| | chlorophenyl)methyl)-3- | U.S. Pat. No. 19890822 | | |
| | ((1,1dimethylethyl)thio)- | | | |
| | alpha, alpha-dimethyl-5- | | | |
| | (2-quinolinylmethoxy)-1H- | | | |
| | Indole-2-propanoic acid | | | |
| Pfizer | 4-(3-(4-(2-Methyl- | | | 5-LPO inhibitor |
| | imidazol-1-yl)- | | | |
| | phenylsulfanyl)-phenyl)- | | | |
| | tetrahydro-pyran-4- | | | |
| | carboxylic acid amide | | | |

In preferred methods of the invention, the agents set forth in the Agent Table can be used for prophylactic and/or therapeutic treatment for diseases and conditions associated with FLAP or with other members of the leukotriene pathway, or with increased leukotriene synthesis. In particular, they can be used for treatment for myocardial infarction or susceptibility to myocardial infarction, such as for individuals in an at-risk population as described above, (e.g., based on identified risk factors such as elevated cholesterol, elevated C-reactive protein, and/or genotype); for individuals suffering from acute coronary syndrome, such as unstable angina, non-ST-elevation myocardial infarction (NSTEMI) or ST-elevation myocardial infarction (STEMI); methods for reducing risk of MI, stroke or PAOD in persons with asymptomatic ankle/brachial index less than 0.9; for decreasing risk of a subsequent myocardial infarction, such as in individuals who have already had one or more myocardial infarctions; for stroke or susceptibility to stroke; for decreasing risk of a second stroke; for PAOD or susceptibility to PAOD; for treatment of atherosclerosis, such as in patients requiring treatment (e.g., angioplasty, stents, revascularization procedure) to restore blood flow in arteries (e.g., coronary, carotid, and/or femoral arteries); for treatment of asymptomatic ankle/brachial index of less than 0.9; and/or for decreasing leukotriene synthesis (e.g., for treatment of myocardial infarction, ACS, stroke or PAOD

In one preferred embodiment of the invention, the leukotriene synthesis inhibitor is an inhibitor of FLAP such as 1-((4-chlorophenyl)methyl)-3-((1,1-dimethylethyl)thio)-alpha,alpha-dimethyl-5-(2-quinolinylmethoxy)-1H-Indole-2-propanoic acid otherwise known as MK-0591, (R)-(+)-alpha-cyclopentyl-4-(2-quinolinylmethoxy)-Benzeneacetic acid otherwise known as BAY-x-1005, 3-(3-(1,1-dimethylethylthio-5-(quinoline-2-ylmethoxy)-1-(4-chloromethylphenyl)indole-2-yl)-2,2-dimethylpropionaldehyde oxime-0-2-acetic acid otherwise known as A-81834, their optically pure enantiomers, salts, chemical derivatives, analogues, or other compounds inhibiting FLAP that effectively decrease leukotriene biosynthesis when administered to humans.

In another preferred embodiment of the invention, the leukotriene synthesis inhibitor is an inhibitor of 5 LO such as zileuton, atreleuton, 6-((3-fluoro-5-(tetrahydro-4-methoxy-2H-pyran-4yl)phenoxy)methyl)-1-methyl-2(1H)-quinlolinone otherwise known as ZD-2138, 1-((4-chlorophenyl)methyl)-3-((1,1dimethylethyl)thio)-alpha,alpha-dimethyl-5-(2-quinolinylmethoxy)-1H-Indole-2-propanoic acid otherwise known as MK-886, 4-(3-(4-(2-Methyl-imidazol-1-yl)-phenylsulfanyl)-phenyl)-tetrahydro-pyran-4-carboxylic acid amide otherwise known as CJ-13610, their optically pure enantiomers, salts, chemical derivatives, analogues or other compounds inhibiting 5-LO that effectively decrease leukotriene biosynthesis when administered to humans.

The compound can be represented by the following formula: 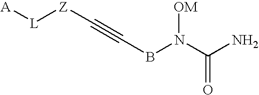
or a pharmaceutically acceptable salt thereof, wherein M is selected from the group consisting of hydrogen, a pharmaceutically acceptable cation, and a pharmaceutically acceptable metabolically cleavable group; B is a straight or branched divalent alkylene group of from one to twelve carbon atoms; Z is thiazolyl, optionally substituted with alkyl of from one to six carbon atoms or haloalkyl of from one to six carbon atoms; L is selected from the group consisting of (a) alkylene of from 1-6 carbon atoms, (b) alkenylene of from 2-6 carbon atoms, (c) alkynylene of from 2-6 carbon atoms, (d) hydroxyalkyl of 1-6 carbon atoms, (e) >C═O, (f) >C═N—OR₁, where R₁ is hydrogen or C₁-C₆ alkyl, (g) —(CHR1)ₙ (CO)(CHR₂)ₘ, where n and m are independently selected from an integer from one to six and R₁ and R₂ are independently selected from hydrogen and C₁-C₆ -alkyl, (h) —(CHR1)ₙ C═NOR₂, where R₁, R₂ and n are as defined above; (i) —(CHR1)ₙ ON═CR₂, where R₁, R₂ and n are as: defined above; (j) —(CHR₁)ₙ—O—(CHR₂)ₘ—, where R₁, R₂, n and m are as defined above, (k) —(CHR₁)ₙ—NR₂ (CHR₃)ₘ—, where R₁, R₂, n and m are as defined above and R₃ is selected from hydrogen and C₁-C₆-alkyl; (l) —(CHR₁)ₙ—S—CHR₂)ₘ—, where R₁, R₂, n and m are as defined above; and (m) —(CHR₁)ₙ—(SO₂)—(CHR₂)ₘ—, where R₁, R₂, n and m are as defined above; A is carbocyclic aryl optionally substituted with alkyl of from one to six carbon atoms, haloalkyl of from one to six carbon atoms, hydroxyalkyl of from one to six carbon atoms, alkoxy of from one to twelve carbon atoms, alkoxyalkoxyl in which the two alkoxy portions may each independently contain from one to six carbon atoms, alkylthio of from one to six carbon atoms, hydroxy, halogen, cyano, amino, alkylamino of from one to six carbon atoms, dialkylamino in which the two alkyl groups may independently contain from one to six carbon atoms, alkanoylamino of from two to eight carbon atoms, N-alkanoyl-N-alkylamino in which the alkanoyl is of from two to eight carbon atoms and the alkyl group is of from one to six carbon atoms, alkylaminocarbonyl of from two to eight carbon atoms, dialkylaminocarbonyl in which the two alkyl groups are independently of from one to six carbon atoms, carboxyl, alkoxycarbonyl or from two to eight carbon atoms, phenyl, optionally substituted with alkyl of from one to six carbon atoms, haloalkyl of from one to six carbon atoms, alkoxy of from one to six carbon atoms, hydroxy or halogen, phenoxy, optionally substituted with alkyl of from one to six carbon atoms, haloalkyl of from one to six carbon atoms, alkoxy of from one to six carbon atoms, hydroxy or halogen, and phenylthio, optionally substituted with alkyl of from one to six carbon atoms, haloalkyl of from one to six carbon atoms, alkoxy of from one to six carbon atoms, hydroxy or halogen. Preferably, the compound is a compound or pharmaceutically acceptable salt thereof having the name (R)—N-{3-[-5-(4-fluorophenylmethyl)thiazo-2-yl]-1methyl-2-propynyl}-N-hydroxyurea. See U.S. Pat. No. 4,615,596, incorporated herein by reference.

The compound is represented by the following formula: 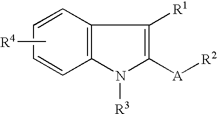
or a pharmaceutically acceptable salt thereof, wherein A is selected from the group consisting of straight or branched divalent alkylene of from one to twelve carbon atoms and divalent cycloalkylene of from three to eight carbon atoms; R₁ is selected from the group consisting of hydrogen, alkylthio of from one to six carbon atoms, phenylthio, optionally substituted with alkyl of from one to six carbon atoms, alkoxy of from one to six carbon atoms, or halogen, phenylalkylthio in which the alkyl portion contains from one to six carbon atoms, and the phenyl group is optionally substituted with alkyl of from one to six carbon atoms, alkoxy of from one to six carbon atoms, or halogen, R₂ is selected from the group consisting of —COOB wherein B is selected from hydrogen, a pharmaceutically acceptable cation, or a metabolically cleavable group, —COOalkyl where the alkyl portion contains from one to six carbon atoms, —COOalkylcarbocyclicaryl where the alkyl portion contains from one to six carbon atoms and the aryl portion is optionally substituted with alkyl of from one to six carbon atoms, alkoxy of from one to six carbon atoms, or halogen, —CONR₅ R₆ wherein R₅ is selected from the group consisting of hydrogen, hydroxyl, alkyl of from one to six carbon atoms, and alkoxy of from one to six carbon atoms, and R₆ is selected from the group consisting of hydrogen and alkyl of from one to six carbon atoms, —COR₆, and —OH; R₃ is selected from the group consisting of phenylalkyl in which the alkyl portion contains from one to six carbon atoms, and the phenyl group is optionally substituted with alkyl of from one to six carbon atoms, alkoxy of from one to six carbon atoms, or halogen, R₄ is selected from the group consisting of thiazolylalkyloxy in which the alkyl portion contains from one to six carbon atoms, and the heteroaryl portion is optionally substituted with alkyl of from one to six carbon atoms, alkoxy of from one to six carbon atoms, or halogen, and thiazolyloxy optionally substituted with alkyl of from one to six carbon atoms, alkoxy of from one to six carbon atoms, or halogen. See U.S. Pat. No. 5,288,743, incorporated herein by reference.

The compound can be represented by the formula: 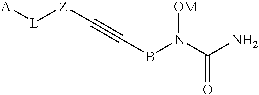
or a pharmaceutically acceptable salt thereof, wherein M is selected from the group consisting of hydrogen, and a pharmaceutically acceptable cation; B is a straight or branched divalent alkylene group of from one to twelve carbon atoms; Z is selected from the group consisting of: (a) furyl, optionally substituted with alkyl of from one to six carbon atoms, or haloalkyl of from one to six carbon atoms, and (b) thienyl, optionally substituted with alkyl of from one to six carbon atoms, or haloalkyl of from one to six carbon atoms; and L is alkylene of from 1-6 carbon atoms; A is phenyl optionally substituted with alkyl of from one to six carbon atoms, haloalkyl of from one to six carbon atoms, hydroxyalkyl of from one to six carbon atoms, alkoxy of from one to twelve carbon atoms, alkoxyalkoxyl in which the two alkoxy portions may each independently contain from one to six carbon atoms, alkylthio of from one to six carbon atoms, hydroxy, halogen, cyano, amino, alkylamino of from one to six carbon atoms, dialkylamino in which the two alkyl groups may independently contain from one to six carbon atoms, alkanoylamino of from two to eight carbon atoms, N-alkanoyl-N-alkylamino in which the alkanoyl is of from two to eight carbon atoms and the alkyl group is of from one to six carbon atoms, alkylaminocarbonyl of from two to eight carbon atoms, dialkylaminocarbonyl in which the two alkyl groups are independently of from one to six carbon atoms, carboxyl, alkoxycarbonyl of from two to eight carbon atoms, phenyl, optionally substituted with alkyl of from one to six carbon atoms, haloalkyl of from one to six carbon atoms, alkoxy of from one to six carbon atoms, hydroxy or halogen, phenoxy, optionally substituted with alkyl of from one to six carbon atoms, haloalkyl of from one to six carbon atoms, alkoxy of from one to six carbon atoms, hydroxy or halogen, or phenylthio, optionally substituted with alkyl of from one to six carbon atoms, haloalkyl of from one to six carbon atoms, alkoxy of from one to six carbon atoms, hydroxy or halogen. Preferably, the compound is a compound or a pharmaceutically acceptable salt thereof selected from the group consisting of: N-{3-(5-(4-fluorophenylmethyl)fur-2-yl)-3-butyn-2-yl}-N-hydroxyurea; N-{3-(5-(4-fluorophenylmethyl)-2-thienyl)-1-methyl-2-propynyl}-N-hydroxyurea; (R)-N-{3-(5-(4-fluorophenylmethyl)-2-thienyl)-1-methyl-2-propynyl}-N-hydroxyurea; and (R)-N-{3-(5-(4-chlorophenylmethyl)-2-thienyl)-1-methyl-2-propynyl}-N-hydroxyurea; (S)-N-{3-[5-(4-fluorophenylmethyl)-2-thienyl]-1-methyl-2-propynyl}-N-hydroxyurea. See U.S. Pat. No.5,288,751, incorporated by reference herein.

The compound can be represented by the formula: 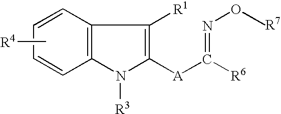
or a pharmaceutically acceptable salt thereof, wherein A is selected from the group consisting of straight or branched divalent alkylene of one to twelve carbon atoms, straight or branched divalent alkenylene of two to twelve carbon atoms, and divalent cycloalkylene of three to eight carbon atoms; R¹ is alkylthio of one to six carbon atoms; R⁶ is selected from the group consisting of hydrogen and alkyl of one to six carbon atoms; R⁷ is selected from the group consisting of (carboxyl)alkyl in which the alkyl portion is of one to six carbon atoms, (alkoxycarbonyl)alkyl in which the alkoxycarbonyl portion is of two to six carbon atoms and the alkyl portion is of one to six carbon atoms, (aminocarbonyl)alkyl in which the alkyl portion is of one to six carbon atoms, ((alkylamino)carbonyl)alkyl in which each alkyl portion independently is of one to six carbon atoms, and ((dialkylamino)carbonyl)alkyl in which each alkyl portion independently is of one to six carbon atoms; R³ is phenylalkyl in which the alkyl portion is of one to six carbon atoms; R⁴ is 2-, 3- or 6-quinolylmethoxy, optionally substituted with alkyl of one to six carbon atoms, haloalkyl of one to six carbon atoms, alkoxy of one to twelve carbon atoms, halogen, or hydroxy. Preferably the compound is selected from the group consisting of: 3-(3-1,1-dimethylethylthio)-5-(quinolin-2-ylmethoxy-1-(4-chlorophenylmethyl)-indol-2-yl)-2,2-dimethylpropionaldehyde oxime-O-2 acetic acid; 3-(3-(1,1-dimethylethylthio)-5-(quinolin-2-ylmethoxy)-1-(4-chloro-phenylmethyl) indol-2-yl)-2,2-dimethylpropionaldehyde oxime-O-2-(3-methyl)butyric acid; 3-(3-(1,1-dimethylethylthio)-5-(6,7-dichloroquinolin-2-ylmethoxy)-1-(4-chlorophenylmethyl)indol-2-yl)-2,2-dimethylpropionaldehyde oxime-O-2-acetic acid; and 3-(3-(1,1-dimethylethylthio)-5-(6-fluoroquinolin-2-ylmethoxy)-1-(4chlorophenylmethyl)indol-2-yl)-2,2-dimethylpropionaldehyde oxime-O-2-propionic acid; or a pharmaceutically acceptable salt or ester thereof. See U.S. Pat. No. 5,459,150, incorporated by reference herein.

The compound can be represented by the formula:
Q¹-X—Ar-Q²

or pharmaceutically acceptable salts thereof, wherein Q is a 9-, 10- or 11-membered bicyclic heterocyclic moiety containing one or two nitrogen heteroatoms and optionally containing a further heteroatom selected from nitrogen, oxygen and sulphur, and Q may optionally bear up to four substituents selected from halogeno, hydroxy, cyano, formyl, oxo, thioxo, (1-4C)alkyl, (3-4C)alkenyl, (3-4C)alkynyl, (1-4C)alkoxy, fluoro-(1-4C)alkyl, hydroxy-(1-4C)alkyl, (2-5C)alkanoyl, phenyl, benzoyl and benzyl, and wherein said phenyl, benzoyl and benzyl substituents may optionally bear one or two substituents selected from halogeno, (1-4C)alkyl and (1-4C)alkoxy;
X is oxy, thio, sulphinyl or sulphonyl; Ar is phenylene, pyridinediyl, pyrimidinediyl, thiophenediyl, furandiyl, thiazolediyl, oxazolediyl, thiadiazolediyl or oxadiazolediyl which may optionally bear one or two substituents selected from halogeno, cyano, trifluoromethyl, hydroxy, amino, (1-4C)alkyl, (1-4C)alkoxy, (1-4C)alkylamino and di-(1-4C)alkylamino; and Q is selected from the groups of the formulae II and III:

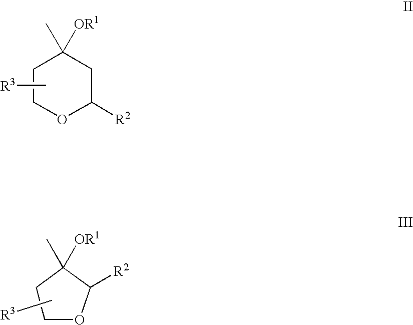

wherein R is hydrogen, (2-5C)alkanoyl or benzoyl, and wherein said benzoyl group may optionally bear one or two substituents selected from halogeno, (1-4C)alkyl and (1-4C)alkoxy; R is (1-4C)alkyl; and R is hydrogen or (1-4C)alkyl; or R and R are linked to form a methylene, vinylene, ethylene or trimethylene group. Preferably, the compound is selected from the group consisting of: (2S,4R)-4-[5-fluoro-3-(1-methyl-2-oxo-1,2,3,4-tetrahydroquinolin-6-ylthio)phenyl]-4-hydroxy-2-ethyltetrahydropyran, (2S,4R)-4-[5-fluoro-3-(1-methyl-2-oxo-1,2,3,4-tetrahydroquinolin-6-ylsulphonyl)phenyl]-4-hydroxy-2-methyltetrahydropyran, (2S,4R)-4-hydroxy-2-methyl-4-[2-(1-methyl-2-oxo-1,2,3,4-tetrahydroquinolin-6-ylthio)thiazol-5-yl]tetrahydropyran, (2S,4R)-4-hydroxy-2-methyl-4-[2-(1-methyl-2-oxo-1,2,3,4-tetrahydroquinolin-6-ylsulphonyl)thiazol-5-yl]tetrahydropyran, (2S,4R)-4-[2-(7-fluoro-1-methyl-2-oxo-1,2,3,4-tetrahydroquinolin-6-ylthio)thiazol-5-yl]-4-hydroxy-2-methyltetrahydropyran, (2S,4R)-4-hydroxy-2-methyl-4-[2-(1-methyl-2-oxoindolin-5-ylthio)thiazol-5-yl]tetrahydropyran, (2S,4R)-4-hydroxy-2-methyl-4-[2-(1-methyl-2-oxo-1,2,3,4-tetrahydroquinolin-6-ylthio)thien-4-yl]tetrahydropyran, (2S,4R)-4-hydroxy-2-methyl-4-[2-(1-methyl-2-oxo-1,2,3,4-tetrahydroquinolin-6-ylsulphonyl)thien-4-yl]tetrahydropyran, (2S,4R)-4-hydroxy-2-methyl-4-[2-(1-methyl-2-oxo-1,2,3,4-tetrahydroquinolin-6-ylthio)thien-5-yl]tetrahydropyran, (2S,4R)-4-hydroxy-2-methyl-4-[2-(1-methyl-2-oxo-1,2-dihydroquinolin-6-ylthio)thien-4-yl]tetrahydropyran, (2S,4R)-4-hydroxy-2-methyl-4-[2-(1,8-dimethyl-2-oxo-1,2,3,4-tetrahydroquinolin-6-ylthio)thien-4-yl]tetrahydropyran, 4-[2-(8-fluoro-1-methyl-2-oxo-1,2,3,4-tetrahydroquinolin-6-ylthio)thien-4-yl]-4-hydroxy-2-methyltetrahydropyran, 4-[2-(7-fluoro-1-methyl-2-oxo-1,2,3,4-tetrahydroquinolin-6-ylthio)thien-4-yl]-4-hydroxy-2-methyltetrahydropyran, (2S,4R)-4-hydroxy-2-methyl-4-[2-(1-methyl-2-oxoindolin-5-ylthio)thien-4-yl]tetrahydropyran, (2S,4R)-4-hydroxy-2-methyl-4-[3-(1-methyl-2-oxo-1,2,3,4-tetrahydroquinolin-6-ylthio)phenyl]tetrahydropyran, (2S,4R)-4-hydroxy-2-methyl-4-[3-(1-methyl-2-oxo-1,2,3,4-tetrahydroquinolin-6-ylsulphonyl)phenyl]tetrahydropyran, (2S,4R)-4-[3-(1-ethyl-2-oxo-1,2,3,4-tetrahydroquinolin-6-ylthio)phenyl]-4-hydroxy-2-methyltetrahydropyran, (2S,4R)-4-[3 -(7-fluoro-1-methyl-2-oxo-1,2,3,4-tetrahydroquinolin-6-ylthio)phenyl]-4-hydroxy-2-methyltetrahydropyran, (2S,4R)-4-hydroxy-2-methyl-4-[3-(1-methyl-2-oxo-1,2-dihydroquinolin-6-ylthio)phenyl]tetrahydropyran, (2S,4R)-4-[3 -(8-chloro-1-methyl-2-oxo-1,2,3,4-tetrahydroquinolin-6-ylthio)phenyl]-4-hydroxy-2-methyltetrahydropyran and (2S,4R)-4-hydroxy-2-methyl-4-[3-(1-methyl-2-oxoindolin-5-ylthio)phenyl]tetrahydropyran. See EP 623614 B1, incorporated herein by reference.

The compound can be represented by the formula:

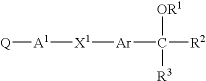

wherein Q is a 10-membered bicyclic heterocyclic moiety containing one or two nitrogen heteroatoms which bears one or two thioxo substituents, and which heterocyclic moiety may optionally bear one, two or three further substituents selected from halogeno, hydroxy, cyano, amino, (1-4C)alkyl, (1-4C)alkoxy, fluoro-(1-4C)alkyl, (1-4C)alkylamino, di-[(1-4C)alkyl]amino, amino-(1-4C)alkyl, (1-4C)alkylamino-(1-4C)alkyl, di-[(1-4C)alkyl]amino-(1-4C)alkyl, phenyl and phenyl-(1-4C)alkyl, and wherein said phenyl or phenyl-(1-4C)alkyl substituent may optionally bear a substituent selected from halogeno, (1-4C)alkyl and (1-4C)alkoxy;
wherein A is a direct link to X or is (1-3C)alkylene; wherein X is oxy, thio, sulphinyl, sulphonyl or imino; wherein Ar is phenylene which may optionally bear one or two substituents selected from halogeno, hydroxy, amino, nitro, cyano, carbamoyl, ureido, (1-4C)alkyl, (1-4C)alkoxy, (1-4C)alkylamino, di-[(1-4C)alkyl]amino, fluoro-(1-4C)alkyl and (2-4C)alkanoylamino; or Ar is pyridylene; wherein R is (1-4C)alkyl, (3-4C)alkenyl or (3-4C)alkynyl; and wherein R and R together form a group of the formula -A-X-A- which, together with the carbon atom to which A and A are attached, defines a ring having 5 to 7 ring atoms, wherein A and A, which may be the same or different, each is (1-3C)alkylene and X is oxy, thio, sulphinyl or sulphonyl, and which ring may bear one, two or three substituents, which may be the same or different, selected from hydroxy, (1-4C)alkyl and (1-4C)alkoxy; or wherein R and R together form a group of the formula -A-X-A- which, together with the oxygen atom to which A is attached and with the carbon atom to which A is attached, defines a ring having 5 to 7 ring atoms, wherein A and A, which may be the same or different, each is (1-3C)alkylene and X is oxy, thio, sulphinyl or sulphonyl, and which ring may bear one, two or three (1-4C)alkyl substituents, and wherein R is (1-4C)alkyl, (2-4C)alkenyl or (2-4C)alkynyl; or a pharmaceutically-acceptable salt thereof. Preferably, the compound is selected from the group consisting of: 4-(5-fluoro-3-(1-methyl-2-thioxo-1,2-dihydroquinolin-6-ylmethoxy)phenyl]-4-ethoxytetrahydropyran and 4-(5-fluoro-3-(1-methyl-2-thioxo-1,2,3,4-tetrahydroquinolin-6-lmethoxy)phenyl]-4-methoxytetrahydropyran, 4-(5-fluoro-3-(1-methyl-2-thioxo-1,2,3,4-tetrahydroquinolin-6-ylthio)phenyl]-4-methoxytetrahydropyran and pharmaceutically-acceptable salt thereof. See EP 466452 B1, incorporated herein by reference.

The compound can be a substituted 4-(quinolin-2-61-methoxy)phenylacetic acid derivative represented by the following formula:

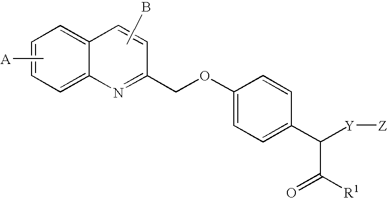

or pharmaceutically acceptable salt thereof, wherein R¹ represents a group of the formula:

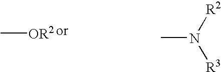

R² and R³ are identical or different and represent hydrogen, lower alkyl, phenyl, benzyl or a group of the formula:

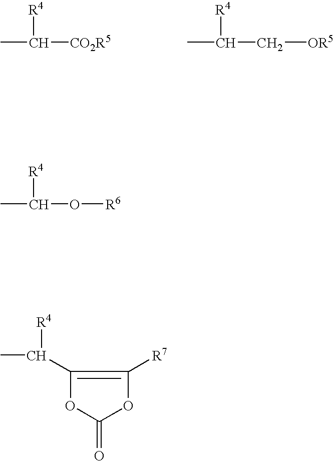

R⁴ represents hydrogen, lower alkyl, phenyl or benzyl, which can optionally be substituted by hydroxyl, carboxyl, lower alkoxycarbonyl, lower alkylthio, heteroaryl or carbamoyl, R⁵ represents hydrogen, lower alkyl, phenyl or benzyl, R⁶ represents a group of the formula —COR⁵ or —CO² R⁵, R⁷ represents hydrogen, lower alkyl or phenyl, Y represents a group of the formula:

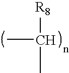

wherein R⁸ represents hydrogen, lower alkyl or phenyl and n denotes a number of 0 to 5, Z represents norbornyl, or represents a group of the formula:

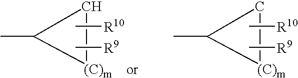

wherein R⁹ and R¹⁰ are identical or different and denote hydrogen, lower alkyl or phenyl, or R⁹ and R¹⁰ can together form a saturated carbocyclic ring having up to 6 carbon atoms and m denotes a number from 1 to 6, and A and B are identical or different and denote hydrogen, lower alkyl or halogen, or a pharmaceutically acceptable salt thereof. Preferably the compounds are selected from the group consisting of: 2-[4-(quinolin-2-yl-methoxy)phenyl]-2-cyclopentylacetic acid, 2-[4-(quinolin-2-yl-methoxy)phenyl]-2-cyclohexylacetic acid, and 2-[4-(quinolin-2-yl-methoxy)phenyl]-2-cycloheptylacetic acid, (+)-enantiomer of 2-[4-(quinolin-2-yl-methoxy)phenyl]-2-cyclopentylacetic acid, (−)-enantiomer of 2-[4-(quinolin-2-yl-methoxy)phenyl]-2-cyclopentylacetic acid and pharmaceutically acceptable salts thereof. See U.S. Pat. No. 4,970,215, incorporated herein by reference.

The compound can be represented by the formula:

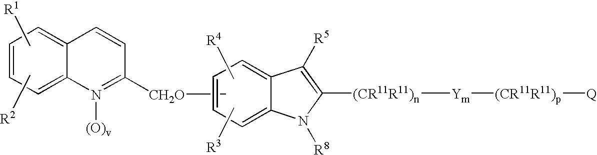

wherein R, R, R, R and R are independently hydrogen, halogen, lower alkyl, lower alkenyl, lower alkynyl, —CF3, —CN, —NO2, —N3, —C(OH)RR, —CO2R, —SR, —S(O)R, —S(O)2R, —S(O)2NRR,—OR,—NRR, —C(O)R or —(CH2)tR; R is hydrogen, —CH3, —CF3, —C(O)H, X—R or X—R; R and R are independently: alkyl, —(CH2)uPh(R)2 or —(CH2)uTh(R)2; R is —CF3 or R; R is hydrogen or X—R; each R is independently hydrogen or lower alkyl, or two R's on same carbon atom are joined to form a cycloalkyl ring of 3 to 6 carbon atoms; R is hydrogen, lower alkyl or —CH2R;
R is lower alkyl or —(CH2)rR; R is —CF3 or R; R is hydrogen, —C(O)R, R, or two R's on the same nitrogen may be joined to form a monocyclic heterocyclic ring of 4 to 6 atoms containing up to 2 heteroatoms chosen from O, S or N; R is hydrogen, —CF3, lower alkyl, lower alkenyl, lower alkynyl or —(CH2)rR; R is —(CH2)s-C(RR)—(CH2)s-R or —CH2C(O)NRR; R is hydrogen or lower alkyl; R is a) a monocyclic or bicyclic heterocyclic ring containing from 3 to 9 nuclear carbon atoms and 1 or 2 nuclear hetero-atoms selected from N, S or O and with each ring in the heterocyclic radical being formed of 5 or 6 atoms, or b) the radical W—R; R is alkyl or C(O)R;
R is phenyl substituted with 1 or 2 R groups; R is hydrogen, halogen, lower alxyl, lower alkoxy, lower alkylthio, lower alkylsulfonyl, lower alkylcarbonyl, —CF3, —CN,
—NO2 or —N3; R is alkyl, cycloalkyl, monocyclic monoheterocyclic ring;
R is the residual structure of a standard amino acid, or R and R attached to the same N can cyclize to form a proline residue; m is 0 to 1; n is 0 to 3; p is 1 to 3 when m is 1; p is 0 to 3 when m is 0; r is 0 to 2; s is 0 to 3; t is 0 to 2; u is 0 to 3; v is 0 or 1;
W is 0, S or NR; X is 0, or NR; X is C(O), CRR, S, S(O) or S(O)2; X is C(O), CRR, S(O)2 or a bond; Y is X or X; Q is —CO2R, —C(O)NHS(O)2R, —NHS(O)2R, —S(O)2NHR —C(O)NRR, —CO2R, —C(O)NRR, —CH2OH, or 1H— or 2H-tetrazol-5-yl;
and the pharmaceutically acceptable salts thereof. Preferred embodiments of the compounds are selected from the following and pharmaceutically acceptable salts thereof:
   3-[N-(p-chlorobenzyl)-3-(t-butylthio)-5-(quinolin-2-ylmethoxy)indol-2-yl]-2,2-dimethylpropanoic acid;
      3-[N-(p-chlorobenzyl)-3 -methyl-5-(quinolin-2-ylmethoxy)indol-2-yl]-2,2-dimethylpropanoic acid;
      3-[N-(p-t-butylthiobenzyl)-3-(t-butylthio)-5-(quinolin-2-ylmethoxy)indol-2-yl]-2,2-dimethylpropanoic acid;
      3-[N-(p-chlorobenzyl)-3-(phenylthio)-5-(quinolin-2-ylmethoxy)indol-2-yl]-2,2-dimethylpropanoic acid;
      3-[N-(p-chlorobenzyl)-3-(phenylsulfonyl)-5-(quinolin-2-ylmethoxy)indol-2-yl]-2,2-dimethyl propanoic acid, N-oxide;
      3-[N-(p-chlorobenzyl)-3-(phenylsulfonyl)-5-(quinolin-2-ylmethoxy)indol-2-yl]-2,2-dimethylpropanoic acid;
      3-[N-(p-chlorobenzyl)-3-(phenylsulfinyl)-5-(quinolin-2-ylmethoxy)indol-2-yl]-2,2-dimethylpropanoic acid;
      3-[N-(p-chlorobenzyl)-5-(quinolin-2-ylmethoxy)indol-2-yl]-2,2-dimethylpropanoic acid;
      3-[N-(p-chlorobenzyl)-3-benzoyl-5-(quinolin2-ylmethoxy)indol-2-yl]-2,2-dimethylpropanoic acid;
      3-[N-(p-chlorobenzyl)-3-benzyl-5-(quinolin-2-ylmethoxy)indol-2-yl]-2,2-dimethylpropanoic acid;
      3-[N-(p-chlorobenzyl)-3-(3,3-dimethyl-1-oxo-1-butyl)-5-(quinolin-2-ylmethoxy)indol-2-yl]-2,2-dimethylpropanoic acid;
      2-[N-(p-chlorobenzyl)-3-(t-butylthio)-5-(quinolin-2-ylmethoxy)indol-2-yl]ethoxyethanoic acid;
      3-[N-(p-chlorobenzyl)-3-(3,3-dimethyl-1-butyl)-5-(quinolin-2-ylmethoxy)indol-2-yl]-2,2-dimethylpropanoic acid;
      3-[N-(p-chlorobenzyl)-3-(t-butylthio)-5-(quinolin-2-ylmethoxy)indol-2-yl]-2-methylpropanoic acid;
      3-[N-(p-chlorobenzyl)-3-methyl-5-(6,7-dichloroquinolin-2-ylmethoxy)indol-2-yl]-2,2-dimethylpropanoic acid;
      3-[N-(p-chlorobenzyl)-3-methyl-5-(7-chloroquinolin-2-ylmethoxy)indol-2-yl]-2,2-dimethylpropanoic acid;
      3-[N-(p-chlorobenzyl)-4-allyl-5-(quinolin-2-ylmethoxy)-3-(t-butylthio)indol-2-yl]-2,2-dimethylpropanoic acid;
      3-[N-(p-chlorobenzyl)-4-allyl-5-(quinolin-2-ylmethoxy)indol-2-yl]-2,2-dimethylpropanoic acid;
      3-[N-(p-chlorobenzyl)-6-(quinolin-2-ylmethoxy)-3-(t-butylthio)indol-2-yl]-2,2-dimethylpropanoic acid;
      3-[N-(p-chlorobenzyl)-4-(quinolin-2-ylmethoxy)-3-(t-butylthio)indol-2-yl]-2,2-dimethylpropanoic acid;
      3-[N-(p-chlorobenzyl)-7-(quinolin-2-ylmethoxy)-3-(t-butylthio)indol-2-yl]-2,2-dimethylpropanoic acid;
      2-[2-[N-(p-chlorobenzyl)-3-(t-butylthio)-5-(quinolin-2-ylmethoxy)indol-2-yl]ethoxy]propanoic acid;
      3-[N-(p-chlorobenzyl)-4-(quinolin-2-ylmethoxy)indol-2-yl]-2,2-dimethylpropanoic acid;
      3-[N-methyl-3-(p-chlorobenzoyl)-6-(quinolin-2-ylmethoxy)indol-2-yl]-2,2-dimethylpropanoic acid,
      3-[N-methyl-3-(p-chlorobenzyl)-6-(quinolin-2-ylmethoxy)indol-2-yl]-2,2-dimethylpropanoic acid,
      3-[N-(4-chlorobenzyl)-3-i-propoxy-5-(quinolin-2-ylmethoxy)indol-2-yl]-2,2-dimethylpropanoic acid,
      3-[N-(4-chlorobenzyl)-3-(t-butylthio)-5-(quinolin-2-yl-methoxy)indol-2-yl]-2-ethylpropanoic acid,
      3-[N-(4-chlorobenzyl)-3-trifluoroacetyl-5-(quinolin-2-ylmethoxy)indol-2-yl]-2,2-dimethylpropanoic acid,
      3-[N-(4-chlorobenzyl)-3-(3,3-dimethyl-1-oxo-1-butyl)-5-(quinolin-2-ylmethoxy)indol-2-yl]-2-methylpropanoic acid,
      3-[3-(3,3-dimethyl-1-oxo-1-butyl-5-(quinolin-2-ylmethoxy)indol-2-yl]-2,2-dimethylpropanoic acid,
      3-[N-(4-triflouromethylbenzyl)-3-(3,3-dimethyl-1-oxo-1-butyl)-5-(quinolin-2-yl-methoxy)indol-2-yl]-2,2-dimethylpropanoic acid,
      3-[N-benzyl-3-(3,3-dimethyl-1-oxo-1-butyl)-5-(quinolin-2-ylmethoxy)indol-2-yl]-2,2-dimethylpropanoic acid,
      3-[N-(3-methoxybenzyl)-3-(3,3-dimethyl-1-oxo-1-butyl)-5-(quinolin-2-ylmethoxy)indol-2-yl]-2,2-dimethylpropanoic acid,
      3-[N-allyl-3-(3,3-dimethyl-1-oxo-1-butyl)-5-(quinolin-2-ylmethoxy)indol-2-yl]-2,2-dimethylpropanoic acid,
      3-[N-(4-methoxybenzyl)-3-(3,3-dimethyl-1-oxo-1-butyl)-5-(quinolin-2-ylmethoxy)indol-2-yl]-2,2-dimethylpropanoic acid,
      3-[N-methyl-3-(3,3-dimethyl-1-oxo-3-butyl)-5-(quinolin-2-ylmethoxy)indol-2-yl]-2,2-dimethylpropanoic acid,
      3-[3-(4-chlorobenzyl)-6-(quinolin-2-ylmethoxy)indol-2-yl]-2,2-dimethylpropanoic acid.
      3-[N-(phenylsulfonyl)-3-(4-chlorobenzyl)-6-(quinolin-2-ylmethoxy)indol-2-yl]-2,2-dimethylpropanoic acid,
      3-[N-benzyl-3-(4-chlorobenzyl)-6-(quinolin-2-ylmethoxy)indol-2-yl]-2,2-dimethylpropanoic acid,
      3-[N-(4-chlorobenzyl)-3-(t-butylsulfonyl)-5-(quinolin-2-ylmethoxy)indol-2-yl]-2,2-dimethylpropanoic acid,
      3-[N-(4-chlorobenzyl)-3-(t-butylsulfinyl)-5-(quinolin-2-ylmethoxy)indol-2-yl]-2,2-dimethylpropanoic acid,
      3-[N-allyl-3-(4-chlorobenzyl)-6-(quinolin-2-ylmethoxy)indol-2-yl]-2,2-dimethylpropanoic acid,
      3-[N-(n-propyl)-3-(4-chlorobenzyl)-6-(quinoline-2-ylmethoxy)indol-2-yl]-2,2-dimethylpropanoic acid,
      3-[N-ethyl-3-(4-chlorobenzyl)-6-(quinolin-2-ylmethoxy)indol-2-yl]-2,2-dimethylpropanoic acid,
      3-[N-(4-chlorobenzyl)-3-(4-t-butylbenzoyl)-5-(quinolin-2-yl-methoxy)indol-2-yl]-2,2-dimethylpropanoic acid,
      3-[N-(4-chlorobenzyl)-3-(4-chlorobenzoyl)-5-(quinolin-2-ylmethoxy)indol-2-yl]-2,2-dimethylpropanoic acid,
      3-[N-(4-chlorobenzyl)-3-(1,1-dimethylethyl)-5-(quinolin-2-ylmethoxy)indol-2-yl]-2,2-dimethylpropanoic acid,
      3-[N-(4-chlorobenzyl)-3-acetyl-5-(quinolin-2-ylmethoxy)indol-2-yl]-2,2-dimethylpropanoic acid
      3-[N-(4-chlorobenzyl)-3-cyclopropanecarbonyl-5-(quinolin-2-ylmethoxy)indol-2-yl]-2,2-dimethylpropanoic acid,
      3-[N-(4-chlorobenzyl)-3-(3-cyclopentylpropanoyl)-5-(quinolin-2-ylmethoxy)indol-2-yl]-2,2-dimethylpropanoic acid,
      3-[N-(4-chlorobenzyl)-3-(3-methylbutanoyl)-5 -(quinolin-2-yl-methoxy)indol-2-yl]-2,2-dimethylpropanoic acid,
      3-[N-(4-chlorobenzyl)-3-propanoyl-5-(quinolin-2-ylmethoxy)indol-2-yl]-2,2-dimethylpropanoic acid,
      3-[N-(4-chlorobenzyl)-3-(2-methylpropanoyl)-5-(quinolin-2-ylmethoxy)indol-2-yl]-2,2-dimethylpropanoic acid,
      3-[N-(4-chlorobenzyl)-3-trimethylacetyl-5-(quinolin-2-ylmethoxy)indol-2-yl]-2,2-dimethylpropanoic acid,
      3-[N-(4-chlorobenzyl)-3-phenylacetyl-5-(quinolin-2-ylmethoxy)indol-2-yl]-2,2-dimethylpropanoic acid,
      3-[N-(4-fluorobenzyl)-3-(3,3-dimethyl-1-oxo-1-butyl)-5-(quinolin-2-ylmethoxy)indol-2-yl]-2,2-dimethylpropanoic acid,
      3-[N-(4-bromobenzyl)-3-(3,3-dimethyl-1-oxo-1-butyl)-5-(quinolin-2-ylmethoxy)indol-2-yl]-2,2-dimethylpropanoic acid,
      3-[N-(4-iodobenzyl)-3-(3,3-dimethyl-1-oxo-1-butyl)-5-(quinolin-2-ylmethoxy)indol-2-yl]-2,2-dimethylpropanoic acid,
      3-[N-(4-chlorobenzyl)-3-(1,1-dimethylbutyl)-5-(quinolin-2-ylmethoxy)indol-2-yl]-2,2-dimethylpropanoic acid,
      3-[N-(4-chlorobenzyl)-3-(1,1-dimethylpropyl)-5-(quinolin-2-ylmethoxy)indol-2-yl]-2,2-dimethylpropanoic acid,
      3-[N-(3-fluorobenzyl)-3-(1,1-dimethylethyl)-5-(quinolin-2-ylmethoxy)indol-2-yl]-2,2-dimethylpropanoic acid,
      3-[N-(4-chlorobenzyl)-3-(3-methylethyl)-5-(quinolin-2-ylmethoxy)indol-2-yl]-2,2-dimethylpropanoic acid,
      3-[N-(4-chlorobenzyl)-3-cyclopropyl-5-(quinolin-2-ylmethoxy)indol-2-yl]-2,2-dimethylpropanoic acid,
      3-[N-(4-chlorobenzyl)-3-(1-methyl-1-cyclopropyl)-5-(quinolin-2-ylmethoxy)indol-2-yl]-2,2-dimethylpropanoic acid,
      3-[N-(4-chlorobenzyl)-3-cyclopentyl-5-(quinolin-2-ylmethoxy)indol-2-yl]-2,2-dimethylpropanoic acid,
      3-[N-(4-chlorobenzyl)-3-cyclohexyl-5-(quinolin-2-ylmethoxy)indol-2-yl]-2,2-dimethylpropanoic acid,
      3-[N-(4-chlorobenzyl)-3-(alpha, alpha-dimethylbenzyl)-5-(quinolin-2-ylmethoxy)indol-2-yl]-2,2-dimethylpropanoic acid,
      3-[N-(4-chlorobenzyl)-3-(2-{4-chloro-alpha, alpha-dimethylbenzyl}-5-(quinolin-2-ylmethoxy)indol-2-yl]-2,2-dimethylpropanoic acid,
      3-[N-(4-chlorobenzyl)-3-(1-adamantyl)-5-(quinolin-2-ylmethoxy)indol-2-yl]-2,2-dimethylpropanoic acid,
      3-[N-(4-chlorobenzyl)-3-((1-adamantyl)methyl)-5 -(quinolin-2-ylmethoxy)indol-2-yl]-2,2-dimethylpropanoic acid,
      3-[N-(1,1-dimethylethyl)-3-(4-chlorobenzyl)-6-(quinolin-2-ylmethoxy)indol-2-yl]-2,2-dimethylpropanoic acid,
      3-[N-(1,1-dimethylpropyl)-3-(4-chlorobenzyl)-6-(quinoline-2-ylmethoxy)indol-2-yl]-2,2-dimethylpropanoic acid,
      3-[N-(4-chlorobenzyl)-3-(3,3-dimethyl-1-oxo-1-butyl)-5-(quinolin-2-ylmethoxy)indol-2-yl]-2,2-diethylpropanoic acid,
      methyl 3-[N-(4-chlorobenzyl)-3,6-bis(acetyl)-5-(quinolin-2-ylmethoxy)indol-2-yl]-2,2 dimethyl propanoate or
      methyl 3-[N-(4-chlorobenzyl)-3,6-bis(cyclopropanecarbonyl)-5-(quinolin-2-ylmethoxy)indol-2-yl]-2,2-dimethyl propanoate. See EP 419049 B1, incorporated herein by reference.

The term “alkyl” refers to a monovalent group derived from a straight or branched chain saturated hydrocarbon by the removal of a single hydrogen atom. Alkyl groups are exemplified by methyl, ethyl, n- and iso-propyl, n-, sec-, iso- and tert-butyl, and the like. The term “hydroxyalkyl” represents an alkyl group, as defined above, substituted by one to three hydroxyl groups with the proviso that no more than one hydroxy group may be attached to a single carbon atom of the alkyl group. The term “alkylamino” refers to a group having the structure —NHR′ wherein R′ is alkyl, as previously defined, examples of alkylamino include methylamino, ethylamino, iso-propylamino and the like. The term “alkylaminocarbonyl” refers to an alkylamino group, as previously defined, attached to the parent molecular moiety through a carbonyl group. Examples of alkylaminocarbonyl include methylamino-carbonyl, ethylaminocarbonyl, iso-propylaminocarbonyl and the like. The term “alkylthio” refers to an alkyl group, as defined above, attached to the parent molecular moiety through a sulfur atom and includes such examples as methylthio, ethylthio, propylthio, n-, sec- and tert-butylthio and the like. The term “alkanoyl” represents an alkyl group, as defined above, attached to the parent molecular moiety through a carbonyl group. Alkanoyl groups are exemplified by formyl, acetyl, propionyl, butanoyl and the like. The term “alkanoylamino” refers to an alkanoyl group, as previously defined, attached to the parent molecular moiety through a nitrogen atom. Examples of alkanoylamino include formamido, acetamido, and the like. The term “N-alkanoyl-N-alkylamino” refers to an alkanoyl group, as previously defined, attached to the parent molecular moiety through an aminoalkyl group. Examples of N-alkanoyl-N-alkylamino include N-methylformamido, N-methyl-acetamido, and the like. The terms “alkoxy” or “alkoxyl” denote an alkyl group, as defined above, attached to the parent molecular moiety through an oxygen atom. Representative alkoxy groups include methoxyl, ethoxyl, propoxyl, butoxyl, and the like. The term “alkoxyalkoxyl” refers to an alkyl group, as defined above, attached through an oxygen to an alkyl group, as defined above, attached in turn through an oxygen to the parent molecular moiety. Examples of alkoxyalkoxyl include methoxymethoxyl, methoxyethyoxyl, ethoxyethoxyl and the like. The term “alkoxyalkyl” refers to an alkoxy group, as defined above, attached through an alkylene group to the parent molecular moiety. The term “alkoxycarbonyl” represents an ester group; i.e., an alkoxy group, attached to the parent molecular moiety through a carbonyl group such as methoxycarbonyl, ethoxycarbonyl, and the like. The term “alkenyl” denotes a monovalent group derived from a hydrocarbon containing at least one carbon-carbon double bond by the removal of a single hydrogen atom. Alkenyl groups include, for example, ethenyl, propenyl, butenyl, 1-methyl-2-buten-1-yl and the like. The term “alkylene” denotes a divalent group derived from a straight or branched chain saturated hydrocarbon by the removal of two hydrogen atoms, for example methylene, 1,2-ethylene, 1,1-ethylene, 1,3-propylene, 2,2-dimethylpropylene, and the like. The term “alkenylene” denotes a divalent group derived from a straight or branched chain hydrocarbon containing at least one carbon-carbon double bond. Examples of alkenylene include —CH═CH—, —CH₂ CH═CH—, —C(CH₃)═CH—, —CH₂ CH═CHCH₂—, and the like. The term “cycloalkylene” refers to a divalent group derived from a saturated carbocyclic hydrocarbon by the removal of two hydrogen atoms, for example cyclopentylene, cyclohexylene, and the like. The term “cycloalkyl” denotes a monovalent group derived from a monocyclic or bicyclic saturated carbocyclic ring compound by the removal of a single hydrogen atom. Examples include cyclopropyl, cyclobutyl, cyclopentyl, cyclohexyl, bicyclo[2.2.1]heptanyl, and bicyclo[2.2.2]octanyl. The term “alkynylene” refers to a divalent group derived by the removal of two hydrogen atoms from a straight or branched chain acyclic hydrocarbon group containing a carbon-carbon triple bond. Examples of alkynylene include —CH≡CH—, —CH≡CH—CH₂—, —CH≡CH—CH(CH₃)—, and the like. The term “carbocyclic aryl” denotes a monovalent carbocyclic ring group derived by the removal of a single hydrogen atom from a monocyclic or bicyclic fused or non-fused ring system obeying the “4n+2 p electron” or Huckel aromaticity rule. Examples of carbocyclic aryl groups include phenyl, 1- and 2-naphthyl, biphenylyl, fluorenyl, and the like. The term “(carbocyclic aryl)alkyl” refers to a carbocyclic aryl ring group as defined above, attached to the parent molecular moiety through an alkylene group. Representative (carbocyclic aryl)alkyl groups include phenylmethyl, phenylethyl, phenylpropyl, 1-naphthylmethyl, and the like. The term “carbocyclicarylalkoxy” refers to a carbocyclicaryl alkyl group, as defined above, attached to the parent molecular moiety through an oxygen atom. The term “carbocyclic aryloxyalkyl” refers to a carbocyclic aryl group, as defined above, attached to the parent molecular moiety through an oxygen atom and thence through an alkylene group. Such groups are exemplified by phenoxymethyl, 1- and 2-naphthyloxymethyl, phenoxyethyl and the like. The term “(carbocyclic aryl)alkoxyalkyl” denotes a carbocyclic aryl group as defined above, attached to the parent molecular moiety through an alkoxyalkyl group. Representative (carbocyclic aryl)alkoxyalkyl groups include phenylmethoxymethyl, phenylethoxymethyl, 1- and 2-naphthylmethoxyethyl, and the like. “Carbocyclic arylthioalkyl” represents a carbocyclic aryl group as defined above, attached to the parent molecular moeity through a sulfur atom and thence through an alklyene group and are typified by phenylthiomethyl, 1- and 2-naphthylthioethyl and the like. The term “dialkylamino” refers to a group having the structure —NR′R″ wherein R′ and R″ are independently selected from alkyl, as previously defined. Additionally, R′ and R″ taken together may optionally be —(CH₂)ₖₖ— where kk is an integer of from 2 to 6. Examples of dialkylamino include, dimethylamino, diethylaminocarbonyl, methylethylamino, piperidino, and the like. The term “halo or halogen” denotes fluorine, chlorine, bromine or iodine. The term “haloalkyl” denotes an alkyl group, as defined above, having one, two, or three halogen atoms attached thereto and is exemplified by such groups as chloromethyl, bromoethyl, trifluoromethyl, and the like. The term “hydroxyalkyl” represents an alkyl group, as defined above, substituted by one to three hydroxyl groups with the proviso that no more than one hydroxy group may be attached to a single carbon atom of the alkyl group. The term “phenoxy” refers to a phenyl group attached to the parent molecular moiety through an oxygen atom. The term “phenylthio” refers to a phenyl group attached to the parent molecular moiety through a sulfur atom. The term “pyridyloxy” refers to a pyridyl group attached to the parent molecular moiety through an oxygen atom. The terms “heteroaryl” or “heterocyclic aryl” as used herein refers to substituted or unsubstituted 5- or 6-membered ring aromatic groups containing one oxygen atom, one, two, three, or four nitrogen atoms, one nitrogen and one sulfur atom, or one nitrogen and one oxygen atom. The term heteroaryl also includes bi-or tricyclic groups in which the aromatic heterocyclic ring is fused to one or two benzene rings. Representative heteroaryl groups are pyridyl, thienyl, indolyl, pyrazinyl, isoquinolyl, pyrrolyl, pyrimidyl, benzothienyl, furyl, benzo[b]furyl, imidazolyl, thiazolyl, carbazolyl, and the like. The term “heteroarylalkyl” denotes a heteroaryl group, as defined above, attached to the parent molecular moiety through an alkylene group. The term “heteroaryloxy” denotes a heteroaryl group, as defined above, attached to the parent molecular moiety through an oxygen atom. The term “heteroarylalkoxy” denotes a heteroarylalkyl group, as defined above, attached to the parent molecular moiety through an oxygen atom.

Nucleic Acid Therapeutic Agents

In another embodiment, a nucleic acid of the invention; a nucleic acid complementary to a nucleic acid of the invention; or a portion of such a nucleic acid (e.g., an oligonucleotide as described below); or a nucleic acid encoding a member of the leukotriene pathway (e.g., 5-LO), can be used in “antisense” therapy, in which a nucleic acid (e.g., an oligonucleotide) which specifically hybridizes to the mRNA and/or genomic DNA of a nucleic acid is administered or generated in situ. The antisense nucleic acid that specifically hybridizes to the mRNA and/or DNA inhibits expression of the polypeptide encoded by that mRNA and/or DNA, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct can be delivered, for example, as an expression plasmid as described above. When the plasmid is transcribed in the cell, it produces RNA that is complementary to a portion of the mRNA and/or DNA that encodes the polypeptide for the member of the leukotriene pathway (e.g., FLAP or 5-LO). Alternatively, the antisense construct can be an oligonucleotide probe that is generated ex vivo and introduced into cells; it then inhibits expression by hybridizing with the mRNA and/or genomic DNA of the polypeptide. In one embodiment, the oligonucleotide probes are modified oligonucleotides that are resistant to endogenous nucleases, e.g., exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996, 5,264,564 and 5,256,775). Additionally, general approaches to constructing oligomers useful in antisense therapy are also described, for example, by Van der Krol et al. (Biotechniques 6:958-976 (1988)); and Stein et al. (Cancer Res. 48:2659-2668 (1988)). With respect to antisense DNA, oligodeoxyribonucleotides derived from the translation initiation site are preferred.

To perform antisense therapy, oligonucleotides (mRNA, cDNA or DNA) are designed that are complementary to mRNA encoding the polypeptide. The antisense oligonucleotides bind to mRNA transcripts and prevent translation. Absolute complementarity, although preferred, is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid, as described in detail above. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al., Proc. Natl. Acad. Sci. USA 86:6553-6556 (1989); Lemaitre et al., Proc. Natl. Acad. Sci. USA 84:648-652 (1987); PCT International Publication No. WO 88/09810) or the blood-brain barrier (see, e.g., PCT International Publication No. WO 89/10134), or hybridization-triggered cleavage agents (see, e.g., Krol et al., BioTechniques 6:958-976 (1988)) or intercalating agents. (See, e.g., Zon, Pharm. Res. 5: 539-549 (1988)). To this end, the oligonucleotide may be conjugated to another molecule (e.g., a peptide, hybridization triggered cross-linking agent, transport agent, hybridization-triggered cleavage agent).

The antisense molecules are delivered to cells that express the member of the leukotriene pathway in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in a preferred embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single stranded RNAs that will form complementary base pairs with the endogenous transcripts and thereby prevent translation of the mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systemically).

In another embodiment of the invention, small double-stranded interfering RNA (RNA interference (RNAi)) can be used. RNAi is a post-transcription process, in which double-stranded RNA is introduced, and sequence-specific gene silencing results, though catalytic degradation of the targeted mRNA. See, e.g., Elbashir, S. M. et al., Nature 411:494-498 (2001); Lee, N. S., Nature Biotech. 19:500-505 (2002); Lee, S-K. et al, Nature Medicine 8(7):681-686 (2002); the entire teachings of these references are incorporated herein by reference.

Endogenous expression of a member of the leukotriene pathway (e.g., FLAP, 5-LO) can also be reduced by inactivating or “knocking out” the gene or its promoter using targeted homologous recombination (e.g., see Smithies et al., Nature 317:230-234 (1985); Thomas & Capecchi, Cell 51:503-512 (1987); Thompson et al., Cell 5:313-321 (1989)). For example, an altered, non-functional gene of a member of the leukotriene pathway (or a completely unrelated DNA sequence) flanked by DNA homologous to the endogenous gene (either the coding regions or regulatory regions of the gene) can be used, with or without a selectable marker and/or a negative selectable marker, to transfect cells that express the gene in vivo. Insertion of the DNA construct, via targeted homologous recombination, results in inactivation of the gene. The recombinant DNA constructs can be directly administered or targeted to the required site in vivo using appropriate vectors, as described above. Alternatively, expression of non-altered genes can be increased using a similar method: targeted homologous recombination can be used to insert a DNA construct comprising a non-altered functional gene, or the complement thereof, or a portion thereof, in place of an gene in the cell, as described above. In another embodiment, targeted homologous recombination can be used to insert a DNA construct comprising a nucleic acid that encodes a polypeptide variant that differs from that present in the cell.

Alternatively, endogenous expression of a member of the leukotriene pathway can be reduced by targeting deoxyribonucleotide sequences complementary to the regulatory region of the member of the leukotriene pathway (i.e., the promoter and/or enhancers) to form triple helical structures that prevent transcription of the gene in target cells in the body. (See generally, Helene, C., Anticancer Drug Des., 6(6):569-84 (1991); Helene, C. et al., Ann. N. Y Acad. Sci. 660:27-36 (1992); and Maher, L. J., Bioassays 14(12):807-15 (1992)). Likewise, the antisense constructs described herein, by antagonizing the normal biological activity of one of the members of the leukotriene pathway, can be used in the manipulation of tissue, e.g., tissue differentiation, both in vivo and for ex vivo tissue cultures. Furthermore, the anti-sense techniques (e.g., microinjection of antisense molecules, or transfection with plasmids whose transcripts are anti-sense with regard to a nucleic acid RNA or nucleic acid sequence) can be used to investigate the role of one or more members of the leukotriene pathway in the development of disease-related conditions. Such techniques can be utilized in cell culture, but can also be used in the creation of transgenic animals.

The therapeutic agents as described herein can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. In addition, a combination of any of the above methods of treatment (e.g., administration of non-altered polypeptide in conjunction with antisense therapy targeting altered mRNA for a member of the leukotriene pathway; administration of a first splicing variant in conjunction with antisense therapy targeting a second splicing variant) can also be used.

The invention additionally pertains to use of such therapeutic agents, as described herein, for the manufacture of a medicament for the treatment of MI, ACS, stroke, PAOD and/or atherosclerosis, e.g., using the methods described herein.

Monitoring Progress of Treatment

The current invention also pertains to methods of monitoring the response of an individual, such as an individual in one of the target populations described above, to treatment with a leukotriene synthesis inhibitor.

Because the level of inflammatory markers can be elevated in individuals who are in the target populations described above, an assessment of the level of inflammatory markers of the individual both before, and during, treatment with the leukotriene synthesis inhibitor will indicate whether the treatment has successfully decreased production of leukotrienes in the arterial vessel wall or in bone-marrow derived inflammatory cells. For example, in one embodiment of the invention, an individual who is a member of a target population as described above (e.g., an individual at risk for MI, ACS, stroke or PAOD, such as an individual who is at-risk due to a FLAP haplotype) can be assessed for response to treatment with a leukotriene synthesis inhibitor, by examining leukotriene levels or leukotriene metabolite levels in the individual. Blood, serum, plasma or urinary leukotrienes (e.g., leukotriene E4, cysteinyl leukotriene 1), or ex vivo production of leukotrienes (e.g., in blood samples stimulated with a calcium ionophore to produce leukotrienes), or leukotriene metabolites, can be measured before, and during or after treatment with the leukotriene synthesis inhibitor. The leukotriene or leukotriene metabolite level before treatment is compared with the leukotriene or leukotriene metabolite level during or after treatment. The efficacy of treatment is indicated by a decrease in leukotriene production: a level of leukotriene or leukotriene metabolite during or after treatment that is significantly lower than the level of leukotriene or leukotriene metabolite before treatment, is indicative of efficacy. A level that is lower during or after treatment can be shown, for example, by decreased serum or urinary leukotrienes, or decreased ex vivo production of leukotrienes, or decreased leukotriene metabolites. A level that is “significantly lower”, as used herein, is a level that is less than the amount that is typically found in control individual(s), or is less in a comparison of disease risk in a population associated with the other bands of measurement (e.g., the mean or median, the highest quartile or the highest quintile) compared to lower bands of measurement (e.g., the mean or median, the other quartiles; the other quintiles).

For example, in one embodiment of the invention, the level of a leukotriene or leukotriene metabolite is assessed in an individual before treatment with a leukotriene synthesis inhibitor; and during or after treatment with the leukotriene synthesis inhibitor, and the levels are compared. A level of the leukotriene or leukotriene metabolite during or after treatment that is significantly lower than the level of the leukotriene or leukotriene metabolite before treatment, is indicative of efficacy of treatment with the leukotriene synthesis inhibitor. In another embodiment, production of a leukotriene or a leukotriene metabolite is stimulated in a first test sample from the individual, using a calcium ionophore, before treatment with a leukotriene synthesis inhibitor, and is also stimulated in a second test sample from the individual, using a calcium ionophore, during or after treatment with the leukotriene synthesis inhibitor, and the level of production in the first test sample is compared with with the level of production of the leukotriene or leukotriene metabolite in the second test sample. A level of the leukotriene or leukotriene metabolite in the second test sample that is significantly lower than the level of the leukotriene or leukotriene metabolite in the first test sample, is indicative of efficacy of treatment with the leukotriene synthesis inhibitor.

In another embodiment of the invention, an individual who is a member of a target population of individuals at risk for MI, ACS, stroke or PAOD (e.g., an individual in a target population described above, such as an individual at-risk due to elevated C-reactive protein) can be assessed for response to treatment with a leukotriene synthesis inhibitor, by examining levels of inflammatory markers in the individual. For example, levels of an inflammatory marker in an appropriate test sample (e.g., serum, plasma or urine) can be measured before, and during or after treatment with the leukotriene synthesis inhibitor. The level of the inflammatory marker before treatment is compared with the level of the inflammatory marker during or after treatment. The efficacy of treatment is indicated by a decrease in the level of the inflammatory marker, that is, a level of the inflammatory marker during or after treatment that is significantly lower (e.g., significantly lower), than the level of inflammatory marker before treatment, is indicative of efficacy. Representative inflammatory markers include: C-reactive protein (CRP), serum amyloid A, fibrinogen, a leukotriene, a leukotriene metabolite (e.g., cysteinyl leukotriene 1), interleukin-6, tissue necrosis factor-alpha, soluble vascular cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, myeloperoxidase (MPO), and N-tyrosine. In a preferred embodiment, the marker is CRP or MPO.

Assessment of Increased Risk

The present invention additionally pertains to methods for assessing an individual (e.g., an individual who is in a target population as described herein, such as an individual who is at risk for MI, ACS, stroke or PAOD), for for an increased risk of MI, ACS, atherosclerosis, stroke, transient ischemic attack, transient monocular blindness, asymptomatic carotid stenosis, PAOD, claudication, or limb ischemia. The methods comprise assessing the level of a leukotriene metabolite (e.g., LTE4, LTD4, LTB4) in the individual, wherein an increased level of leukotriene metabolite is indicative of an increased risk. The level can be measured in any appropriate tissue or fluid sample, such as blood, serum, plasma, or urine. In one particular embodiment, the sample comprises neutrophils. The level of the leukotriene metabolite can be measured by standard methods, such as the methods described herein. For example, in one embodiment, production of a leukotriene metabolite is stimulated in a first test sample from the individual, using a calcium ionophore. The level of production is compared with a control level. The control level is a level that is typically found in control individual(s), such as individual who are not at risk for MI, ACS, stroke or PAOD; alternatively, a control level is the level that is found by comparison of disease risk in a population associated with the lowest band of measurement (e.g., below the mean or median, the lowest quartile or the lowest quintile) compared to higher bands of measurement (e.g., above the mean or median, the second, third or fourth quartile; the second, third, fourth or fifth quintile). A level of production of the leukotriene metabolite that is significantly greater than the control level, is indicative of an increased risk. Individuals at increased risk are candidates for treatments described herein.

Pharmaceutical Compositions

The present invention also pertains to pharmaceutical compositions comprising agents described herein, for example, an agent that is a leukotriene synthesis inhibitor as described herein. For instance, a leukotriene synthesis inhibitor can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devices (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the symptoms, and should be decided according to the judgment of a practitioner and each patient's circumstances. Effective doses may be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is preferred. For the purpose of this invention, unit dosage is intended to mean a dosage that is dependent on the individual pharmacodynamics of each agent and administered in FDA approved dosages in standard time courses.

Nucleic Acids of the Invention

FLAP Nucleic Acids, Portions and Variants

In addition, the invention pertains to isolated nucleic acid molecules comprising a human FLAP nucleic acid. The term, “FLAP nucleic acid,” as used herein, refers to an isolated nucleic acid molecule encoding FLAP polypeptide. The FLAP nucleic acid molecules of the present invention can be RNA, for example, mRNA, or DNA, such as cDNA and genomic DNA. DNA molecules can be double-stranded or single-stranded; single stranded RNA or DNA can be either the coding, or sense strand or the non-coding, or antisense strand. The nucleic acid molecule can include all or a portion of the coding sequence of the gene or nucleic acid and can further comprise additional non-coding sequences such as introns and non-coding 3′ and 5′ sequences (including regulatory sequences, for example, as well as promoters, transcription enhancement elements, splice donor/acceptor sites, etc.).

For example, a FLAP nucleic acid can consist of SEQ ID NOs: 1 or 3 or the complement thereof, or to a portion or fragment of such an isolated nucleic acid molecule (e.g., cDNA or the nucleic acid) that encodes FLAP polypeptide (e.g., a polypeptide such as SEQ ID NO: 2). In a preferred embodiment, the isolated nucleic acid molecule comprises a nucleic acid molecule selected from the group consisting of SEQ ID NOs: 1 or 3, or their complement thereof.

Additionally, the nucleic acid molecules of the invention can be fused to a marker sequence, for example, a sequence that encodes a polypeptide to assist in isolation or purification of the polypeptide. Such sequences include, but are not limited to, those that encode a glutathione-S-transferase (GST) fusion protein and those that encode a hemagglutinin A (HA) polypeptide marker from influenza.

An “isolated” nucleic acid molecule, as used herein, is one that is separated from nucleic acids that normally flank the gene or nucleic acid sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material may be purified to essential homogeneity, for example as determined by PAGE or column chromatography such as HPLC. In certain embodiments, an isolated nucleic acid molecule comprises at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb, including but not limited to 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. An isolated nucleic acid molecule or nucleic acid sequence can include a nucleic acid molecule or nucleic acid sequence that is synthesized chemically or by recombinant means. Therefore, recombinant DNA contained in a vector is included in the definition of “isolated” as used herein. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the nucleic acid in tissue (e.g., human tissue), such as by Northern blot analysis.

The present invention also pertains to nucleic acid molecules which are not necessarily found in nature but which encode a FLAP polypeptide (e.g., a polypeptide having an amino acid sequence comprising an amino acid sequence of SEQ ID NOs: 2), or another splicing variant of a FLAP polypeptide or polymorphic variant thereof. Thus, for example, DNA molecules that comprise a sequence that is different from the naturally occurring nucleic acid sequence but which, due to the degeneracy of the genetic code, encode a FLAP polypeptide of the present invention are also the subjects of this invention. The invention also encompasses nucleotide sequences encoding portions (fragments), or encoding variant polypeptides such as analogues or derivatives of a FLAP polypeptide. Such variants can be naturally occurring, such as in the case of allelic variation or single nucleotide polymorphisms, or non-naturally-occurring, such as those induced by various mutagens and mutagenic processes. Intended variations include, but are not limited to, addition, deletion and substitution of one or more nucleotides that can result in conservative or non-conservative amino acid changes, including additions and deletions. Preferably the nucleotide (and/or resultant amino acid) changes are silent or conserved; that is, they do not alter the characteristics or activity of a FLAP polypeptide. In one preferred embodiment, the nucleotide sequences are fragments that comprise one or more polymorphic microsatellite markers. In another preferred embodiment, the nucleotide sequences are fragments that comprise one or more single nucleotide polymorphisms in a FLAP nucleic acid (e.g., the single nucleotide polymorphisms set forth in Table 3, below).

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequence via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleic acid sequence described herein (e.g., nucleic acid molecules which specifically hybridize to a nucleic acid sequence encoding polypeptides described herein, and, optionally, have an activity of the polypeptide). In one embodiment, the invention includes variants described herein which hybridize under high stringency hybridization conditions (e.g., for selective hybridization) to a nucleic acid sequence comprising a nucleic acid sequence selected from the group consisting of SEQ ID NOs: 1 or 3 or the complement thereof. In another embodiment, the invention includes variants described herein which hybridize under high stringency hybridization conditions (e.g., for selective hybridization) to a nucleic acid sequence encoding an amino acid sequence of SEQ ID NO: 2 or a polymorphic variant thereof. In a preferred embodiment, the variant that hybridizes under high stringency hybridizations has an activity of a FLAP.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity that is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are described in Krause, M. H. and S. A. Aaronson, Methods in Enzymology 200: 546-556 (1991), and in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum extent of mismatching among the sequences that hybridize. Generally, doubling the concentration of SSC results in an increase in Tₘ of −17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 minutes at room temperature; a moderate stringency wash can comprise washing in a prewarmed solution (42° C.) solution containing 0.2×SSC/0.1% SDS for 15 minutes at 42° C.; and a high stringency wash can comprise washing in prewarmed (68° C.) solution containing 0.1×SSC/0.1% SDS for 15 minutes at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent homology or identity of two nucleotide or amino acid sequences can be determined by aligning the sequences for optimal comparison purposes (e.g., gaps can be introduced in the sequence of a first sequence for optimal alignment). The nucleotides or amino acids at corresponding positions are then compared, and the percent identity between the two sequences is a function of the number of identical positions shared by the sequences (i.e., % identity=# of identical positions/total # of positions×100). When a position in one sequence is occupied by the same nucleotide or amino acid residue as the corresponding position in the other sequence, then the molecules are homologous at that position. As used herein, nucleic acid or amino acid “homology” is equivalent to nucleic acid or amino acid “identity”. In certain embodiments, the length of a sequence aligned for comparison purposes is at least 30%, for example, at least 40%, in certain embodiments at least 60%, and in other embodiments at least 70%, 80%, 90% or 95% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one embodiment, parameters for sequence comparison can be set at score=100, wordlength=12, or can be varied (e.g., W=5 or W=20).

Another preferred, non-limiting example of a mathematical algorithm utilized for the comparison of sequences is the algorithm of Myers and Miller, CABIOS 4(1): 11-17 (1988). Such an algorithm is incorporated into the ALIGN program (version 2.0) which is part of the GCG sequence alignment software package (Accelrys, Cambridge, UK). When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci. 10:3-5 (1994); and FASTA described in Pearson and Lipman, Proc. Natl. Acad. Sci. USA 85:2444-8 (1988).

In another embodiment, the percent identity between two amino acid sequences can be accomplished using the GAP program in the GCG software package using either a BLOSUM63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package using a gap weight of 50 and a length weight of 3.

The present invention also provides isolated nucleic acid molecules that contain a fragment or portion that hybridizes under highly stringent conditions to a nucleic acid sequence comprising SEQ ID NO: 1 or 3 or the complement of SEQ ID NO: 1 or 3, and also provides isolated nucleic acid molecules that contain a fragment or portion that hybridizes under highly stringent conditions to a nucleic acid sequence encoding an amino acid sequence of the invention or polymorphic variant thereof. The nucleic acid fragments of the invention are at least about 15, for example, at least about 18, 20, 23 or 25 nucleotides, and can be 30, 40, 50, 100, 200 or more nucleotides in length. Longer fragments, for example, 30 or more nucleotides in length, encoding antigenic polypeptides described herein are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. Such probes and primers include polypeptide nucleic acids, as described in Nielsen et al. (Science 254:1497-1500 (1991)).

A probe or primer comprises a region of nucleic acid that hybridizes to at least about 15, for example about 20-25, and in certain embodiments about 40, 50 or 75, consecutive nucleotides of a nucleic acid of the invention, such as a nucleic acid comprising a contiguous nucleic acid sequence of SEQ ID NOs: 1 or 3 or the complement of SEQ ID Nos: 1 or 3, or a nucleic acid sequence encoding an amino acid sequence of SEQ ID NO: 2 or polymorphic variant thereof. In preferred embodiments, a probe or primer comprises 100 or fewer nucleotides, in certain embodiments, from 6 to 50 nucleotides, for example, from 12 to 30 nucleotides. In other embodiments, the probe or primer is at least 70% identical to the contiguous nucleic acid sequence or to the complement of the contiguous nucleotide sequence, for example, at least 80% identical, in certain embodiments at least 90% identical, and in other embodiments at least 95% identical, or even capable of selectively hybridizing to the contiguous nucleic acid sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided herein. For example, nucleic acid molecules can be amplified and isolated using the polymerase chain reaction and synthetic oligonucleotide primers based on one or more of SEQ ID NOs: 1 or 3, or the complement thereof, or designed based on nucleotides based on sequences encoding one or more of the amino acid sequences provided herein. See generally PCR Technology: Principles and Applications for DNA Amplification (ed. H. A. Erlich, Freeman Press, NY, N.Y., 1992); PCR Protocols: A Guide to Methods and Applications (Eds. Innis et al., Academic Press, San Diego, Calif., 1990); Mattila et al., Nucl. Acids Res. 19:4967 (1991); Eckert et al., PCR Methods and Applications 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics 4:560 (1989), Landegren et al., Science 241:1077 (1988), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The amplified DNA can be labeled, for example, radiolabeled, and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Using these or similar methods, the polypeptide and the DNA encoding the polypeptide can be isolated, sequenced and further characterized.

Antisense nucleic acid molecules of the invention can be designed using the nucleotide sequences of SEQ ID NOs: 1 or 3 and/or the complement of one or more of SEQ ID NOs: 1 or 3 and/or a portion of one or more of SEQ ID NOs: 1 or 3 or the complement of one or more of SEQ ID NOs: 1 or 3 and/or a sequence encoding the amino acid sequences of SEQ ID NOs: 2 or encoding a portion of one or more of SEQ ID NOs: 1 or 3 or their complement. They can be constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

The nucleic acid sequences can also be used to compare with endogenous DNA sequences in patients to identify one or more of the disorders related to FLAP, and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions or nucleic acid regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors

Another aspect of the invention pertains to nucleic acid constructs containing a nucleic acid molecule of SEQ ID NOs: 1 or 3 or the complement thereof (or a portion thereof). Yet another aspect of the invention pertains to nucleic acid constructs containing a nucleic acid molecule encoding an amino acid of SEQ ID NO: 2 or polymorphic variant thereof. The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, such as expression vectors, are capable of directing the expression of genes or nucleic acids to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” or “operatively linked” is intended to mean that the nucleic acid sequence of interest is linked to the regulatory sequence(s) in a manner which allows for expression of the nucleic acid sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, “Gene Expression Technology”, Methods in Enzymology 185, Academic Press, San Diego, Calif. (1990). Regulatory sequences include those which direct constitutive expression of a nucleic acid sequence in many types of host cell and those which direct expression of the nucleic acid sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene or nucleic acid that encodes a selectable marker (e.g., for resistance to antibiotics) is generally introduced into the host cells along with the gene or nucleic acid of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene or nucleic acid will survive, while the other cells die).

A host cell of the invention, such as a prokaryotic host cell or eukaryotic host cell in culture can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous FLAP nucleic acid, or an exogenous nucleic acid encoding a FLAP polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleic acid sequence and polypeptide encoded by the sequence and for identifying and/or evaluating modulators of their activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens and amphibians. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley, Current Opinion in BioTechnology 2:823-829 (1991) and in PCT Publication Nos. WO 90/11354, WO 91/01140, WO 92/0968, and WO 93/04169. Clones of the non-human transgenic animals described herein can also be produced according to the methods described in Wilmut et al., Nature 385:810-813 (1997) and PCT Publication Nos. WO 97/07668 and WO 97/07669.

Polypeptides of the Invention

The present invention also pertains to isolated polypeptides encoded by FLAP nucleic acids (“FLAP polypeptides”), and fragments and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide of the invention comprises an amino acid sequence encoded by a nucleic acid molecule comprising a nucleic acid sequence selected from the group consisting of SEQ ID NO: 1 or 3, or the complement of SEQ ID NO: 1 or 3, or portions thereof, or a portion or polymorphic variant thereof. However, the polypeptides of the invention also encompass fragment and sequence variants. Variants include a substantially homologous polypeptide encoded by the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleic acid sequence selected from the group consisting of SEQ ID NOs: 1 or 3 or their complement, or portions thereof, or having substantial homology to a polypeptide encoded by a nucleic acid molecule comprising a nucleic acid sequence selected from the group consisting of nucleotide sequences encoding SEQ ID NO: 2 or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, and in other embodiments at least about 80-85%, and in others greater than about 90% or more homologous or identical. A substantially homologous amino acid sequence, according to the present invention, will be encoded by a nucleic acid molecule hybridizing to SEQ ID NO: 1 or 3 or portion thereof, under stringent conditions as more particularly described above, or will be encoded by a nucleic acid molecule hybridizing to a nucleic acid sequence encoding SEQ ID NO: 2 or a portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gin, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol. 224:899-904 (1992); de Vos et al., Science 255:306-312 (1992)).

The invention also includes fragments of the polypeptides of the invention. Fragments can be derived from a polypeptide encoded by a nucleic acid molecule comprising SEQ ID NO: 1 or 3, or the complement of SEQ ID NO: 1 or 3 (or other variants). However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example beta-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclasses (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a ligand) in biological fluids. The polypeptides can also be used as markers for cells or tissues in which the corresponding polypeptide is preferentially expressed, either constitutively, during tissue differentiation, or in diseased states. The polypeptides can be used to isolate a corresponding binding agent, e.g., ligand, such as, for example, in an interaction trap assay, and to screen for peptide or small molecule antagonists or agonists of the binding interaction. For example, because members of the leukotriene pathway including FLAP bind to receptors, the leukotriene pathway polypeptides can be used to isolate such receptors.

Antibodies of the Invention

Polyclonal and/or monoclonal antibodies that specifically bind one form of the polypeptide or nucleic acid product (e.g., a polypeptide encoded by a nucleic acid having a SNP as set forth in Table 3), but not to another form of the polypeptide or nucleic acid product, are also provided. Antibodies are also provided which bind a portion of either polypeptide encoded by nucleic acids of the invention (e.g., SEQ ID NO: 1 or SEQ ID NO: 3, or the complement of SEQ ID NO: 1 or SEQ ID NO: 3), or to a polypeptide encoded by nucleic acids of the invention that contain a polymorphic site or sites. The invention also provides antibodies to the polypeptides and polypeptide fragments of the invention, or a portion thereof, or having an amino acid sequence encoded by a nucleic acid molecule comprising all or a portion of SEQ ID NOs: 1 or 3, or the complement thereof, or another variant or portion thereof.

The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4:72 (1983)); the EBV-hybridoma technique (Cole et al., Monoclonal Antibodies and Cancer Therapy, Alan R. Liss, 1985, Inc., pp. 77-96); or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No.5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372 (1991); Hay et al., Hum. Antibod. Hybridomas 3:81-85 (1992); Huse et al., Science 246:1275-1281 (1989); Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin and aequorin, and examples of suitable radioactive material include ¹²⁵I, ¹³¹I, ³⁵S or ³H.

As described above, antibodies to leukotrienes can be used in the methods of the invention. The methods described herein can be used to generate such antibodies for use in the methods.

Diagnostic Assays

The nucleic acids, probes, primers, polypeptides and antibodies described herein can be used in methods of diagnosis of a susceptibility to MI, ACS, stroke or PAOD, or to another disease or condition associated with an MI gene, such as FLAP, as well as in kits useful for diagnosis of a susceptibility to MI, ACS, stroke or PAOD, or to another disease or condition associated with FLAP. In one embodiment, the kit useful for diagnosis of susceptibility to MI, ACS, stroke or PAOD, or to another disease or condition associated with FLAP comprises primers as described herein, wherein the primers contain one or more of the SNPs identified in Table 3.

In one embodiment of the invention, diagnosis of susceptibility to MI, ACS, stroke or PAOD (or diagnosis of susceptibility to another disease or condition associated with FLAP), is made by detecting a polymorphism in a FLAP nucleic acid as described herein. The polymorphism can be an alteration in a FLAP nucleic acid, such as the insertion or deletion of a single nucleotide, or of more than one nucleotide, resulting in a frame shift alteration; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene or nucleic acid; duplication of all or a part of the gene or nucleic acid; transposition of all or a part of the gene or nucleic acid; or rearrangement of all or a part of the gene or nucleic acid. More than one such alteration may be present in a single gene or nucleic acid. Such sequence changes cause an alteration in the polypeptide encoded by a FLAP nucleic acid. For example, if the alteration is a frame shift alteration, the frame shift can result in a change in the encoded amino acids, and/or can result in the generation of a premature stop codon, causing generation of a truncated polypeptide. Alternatively, a polymorphism associated with a disease or condition associated with a FLAP nucleic acid or a susceptibility to a disease or condition associated with a FLAP nucleic acid can be a synonymous alteration in one or more nucleotides (i.e., an alteration that does not result in a change in the polypeptide encoded by a FLAP nucleic acid). Such a polymorphism may alter splicing sites, affect the stability or transport of mRNA, or otherwise affect the transcription or translation of the nucleic acid. A FLAP nucleic acid that has any of the alteration described above is referred to herein as an “altered nucleic acid.”

In a first method of diagnosing a susceptibility to MI, ACS, stroke or PAOD, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, a susceptibility to a disease or condition associated with a FLAP nucleic acid (the “test individual”). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in an MI nucleic acid is present, and/or to determine which splicing variant(s) encoded by the FLAP is present. The presence of the polymorphism or splicing variant(s) can be indicated by hybridization of the nucleic acid in the genomic DNA, RNA, or cDNA to a nucleic acid probe. A “nucleic acid probe,” as used herein, can be a DNA probe or an RNA probe; the nucleic acid probe can contain at least one polymorphism in a FLAP nucleic acid or contains a nucleic acid encoding a particular splicing variant of a FLAP nucleic acid. The probe can be any of the nucleic acid molecules described above (e.g., the nucleic acid, a fragment, a vector comprising the nucleic acid, a probe or primer, etc.).

To diagnose a susceptibility to MI, ACS, stroke or PAOD (or another disease or condition associated with FLAP), the test sample containing a FLAP nucleic acid is contacted with at least one nucleic acid probe to form a hybridization sample. A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of one of SEQ ID NOs: 1 and 3, or the complement thereof or a portion thereof; or can be a nucleic acid encoding all or a portion of one of SEQ ID NO: 2. Other suitable probes for use in the diagnostic assays of the invention are described above (see e.g., probes and primers discussed under the heading, “Nucleic Acids of the Invention”).

The hybridization sample is maintained under conditions that are sufficient to allow specific hybridization of the nucleic acid probe to a FLAP nucleic acid. “Specific hybridization,” as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and FLAP nucleic acid in the test sample, then the FLAP has the polymorphism, or is the splicing variant, that is present in the nucleic acid probe. More than one nucleic acid probe can also be used concurrently in this method. Specific hybridization of any one of the nucleic acid probes is indicative of a polymorphism in the FLAP nucleic acid, or of the presence of a particular splicing variant encoding the FLAP nucleic acid, and is therefore diagnostic for a susceptibility to a disease or condition associated with FLAP (e.g., MI, ACS, stroke or PAOD).

In Northern analysis (see Current Protocols in Molecular Biology, Ausubel, F. et al., eds., John Wiley & Sons, supra) the hybridization methods described above are used to identify the presence of a polymorphism or a particular splicing variant, associated with a susceptibility to a disease or condition associated with FLAP (e.g., MI, ACS, stroke or PAOD). For Northern analysis, a test sample of RNA is obtained from the individual by appropriate means. Specific hybridization of a nucleic acid probe, as described above, to RNA from the individual is indicative of a polymorphism in a FLAP nucleic acid, or of the presence of a particular splicing variant encoded by a FLAP nucleic acid, and is therefore diagnostic for susceptibility to a disease or condition associated with FLAP (e.g., MI, ACS, stroke or PAOD).

For representative examples of use of nucleic acid probes, see, for example, U.S. Pat. No. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry 5, American Chemical Society, p. 1 (1994). The PNA probe can be designed to specifically hybridize to a nucleic acid having a polymorphism associated with a susceptibility to a disease or condition associated with FLAP (e.g., MI). Hybridization of the PNA probe to a FLAP nucleic acid as described herein is diagnostic for the susceptibility to the disease or condition.

In another method of the invention, mutation analysis by restriction digestion can be used to detect an altered nucleic acid, or nucleic acids containing a polymorphism(s), if the mutation or polymorphism in the nucleic acid results in the creation or elimination of a restriction site. A test sample containing genomic DNA is obtained from the individual. Polymerase chain reaction (PCR) can be used to amplify a FLAP nucleic acid (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the alteration or polymorphism in the FLAP nucleic acid, and therefore indicates the presence or absence of the susceptibility to a disease or condition associated with FLAP (e.g., MI, ACS, stroke or PAOD).

Sequence analysis can also be used to detect specific polymorphisms in the FLAP nucleic acid. A test sample of DNA or RNA is obtained from the test individual. PCR or other appropriate methods can be used to amplify the nucleic acid, and/or its flanking sequences, if desired. The sequence of a FLAP nucleic acid, or a fragment of the nucleic acid, or cDNA, or fragment of the cDNA, or mRNA, or fragment of the mRNA, is determined, using standard methods. The sequence of the nucleic acid, nucleic acid fragment, cDNA, cDNA fragment, mRNA, or mRNA fragment is compared with the known nucleic acid sequence of the nucleic acid, cDNA (e.g., one or more of SEQ ID NOs: 1 or 3, and/or the complement of SEQ ID NO: 1 or 3), or a nucleic acid sequence encoding SEQ ID NO: 2 or a fragment thereof) or mRNA, as appropriate. The presence of a polymorphism in the FLAP indicates that the individual has a susceptibility to a disease associated with FLAP (e.g., MI, ACS, stroke or PAOD).

Allele-specific oligonucleotides can also be used to detect the presence of polymorphism(s) in the FLAP nucleic acid, through the use of dot-blot hybridization of amplified oligonucleotides with allele-specific oligonucleotide (ASO) probes (see, for example, Saiki, R. et al., Nature 324:163-166 (1986)). An “allele-specific oligonucleotide” (also referred to herein as an “allele-specific oligonucleotide probe”) is an oligonucleotide of approximately 10-50 base pairs, for example, approximately 15-30 base pairs, that specifically hybridizes to a FLAP nucleic acid, and that contains a polymorphism associated with a susceptibility to a disease or condition associated with FLAP (e.g., MI, ACS, stroke or PAOD). An allele-specific oligonucleotide probe that is specific for particular polymorphisms in a FLAP nucleic acid can be prepared, using standard methods (see Current Protocols in Molecular Biology, supra). To identify polymorphisms in the nucleic acid associated with susceptibility to disease, a test sample of DNA is obtained from the individual. PCR can be used to amplify all or a fragment of a FLAP nucleic acid, and its flanking sequences. The DNA containing the amplified FLAP nucleic acid (or fragment of the nucleic acid) is dot-blotted, using standard methods (see Current Protocols in Molecular Biology, supra), and the blot is contacted with the oligonucleotide probe. The presence of specific hybridization of the probe to the amplified FLAP is then detected. Specific hybridization of an allele-specific oligonucleotide probe to DNA from the individual is indicative of a polymorphism in the FLAP, and is therefore indicative of a susceptibility to a disease or condition associated with FLAP (e.g., MI, ACS, stroke or PAOD).

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as oposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′end, or in the middle), the Tₘ could be increased considerably.

In another embodiment, arrays of oligonucleotide probes that are complementary to target nucleic acid sequence segments from an individual, can be used to identify polymorphisms in a FLAP nucleic acid. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,854 and PCT patent publication Nos. WO 90/15070 and WO 92/10092. These arrays can generally be produced using mechanical synthesis methods or light directed synthesis methods that incorporate a combination of photolithographic methods and solid phase oligonucleotide synthesis methods. See Fodor et al., Science 251:767-777 (1991); Pirrung et al., U.S. Pat. No. 5,143,854; (see also PCT Application WO 90/15070); Fodor et al., PCT Publication WO 92/10092; and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief, a target nucleic acid sequence that includes one or more previously identified polymorphic markers is amplified using well-known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array. In a reverse method, a probe, containing a polymorphism, can be coupled to a solid surface and PCR amplicons are then added to hybridize to these probes.

Although primarily described in terms of a single detection block, e.g., detection of a single polymorphism arrays can include multiple detection blocks, and thus be capable of analyzing multiple, specific polymorphisms. It will generally be understood that detection blocks may be grouped within a single array or in multiple, separate arrays so that varying, optimal conditions may be used during the hybridization of the target to the array. For example, it may often be desirable to provide for the detection of those polymorphisms that fall within G-C rich stretches of a genomic sequence, separately from those falling in A-T rich segments. This allows for the separate optimization of hybridization conditions for each situation.

Additional uses of oligonucleotide arrays for detection of polymorphisms can be found, for example, in U.S. Pat. Nos. 5,858,659 and 5,837,832, the entire teachings of which are incorporated by reference herein. Other methods of nucleic acid analysis can be used to detect polymorphisms in a nucleic acid described herein, or variants encoded by a nucleic acid described herein. Representative methods include direct manual sequencing (Church and Gilbert, Proc. Natl. Acad. Sci. USA 81:1991-1995 (1988); Sanger, F. et al., Proc. Natl. Acad. Sci., USA 74:5463-5467 (1977); Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al., Proc. Natl. Acad. Sci. USA 86:232-236 (1989)), mobility shift analysis (Orita, M. et al., Proc. Natl. Acad Sci. USA 86:2766-2770 (1989)), restriction enzyme analysis (Flavell et al., Cell 15:25 (1978); Geever, et al., Proc. Natl. Acad. Sci. USA 78:5081 (1981)); heteroduplex analysis; chemical mismatch cleavage (CMC) (Cotton et al., Proc. Natl. Acad. Sci. USA 85:4397-4401 (1985)); RNase protection assays (Myers, R. M. et al., Science 230:1242 (1985)); use of polypeptides which recognize nucleotide mismatches, such as E. coli mutS protein; allele-specific PCR, for example.

In one embodiment of the invention, diagnosis of a susceptibility to a disease or condition associated with FLAP (e.g., MI, ACS, stroke or PAOD) can also be made by expression analysis by quantitative PCR (kinetic thermal cycling). This technique utilizing TaqMan® can be used to allow the identification of polymorphisms and whether a patient is homozygous or heterozygous. The technique can assess the presence of an alteration in the expression or composition of the polypeptide encoded by a FLAP nucleic acid or splicing variants encoded by a FLAP nucleic acid. Further, the expression of the variants can be quantified as physically or functionally different.

In another embodiment of the invention, diagnosis of a susceptibility to MI, ACS, stroke or PAOD (or of another disease or condition associated with FLAP) can also be made by examining expression and/or composition of a FLAP polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by a FLAP nucleic acid, or for the presence of a particular variant encoded by a FLAP nucleic acid. An alteration in expression of a polypeptide encoded by a FLAP nucleic acid can be, for example, an alteration in the quantitative polypeptide expression (i.e., the amount of polypeptide produced); an alteration in the composition of a polypeptide encoded by a FLAP nucleic acid is an alteration in the qualitative polypeptide expression (e.g., expression of an altered FLAP polypeptide or of a different splicing variant). In a preferred embodiment, diagnosis of a susceptibility to a disease or condition associated with FLAP is made by detecting a particular splicing variant encoded by that FLAP variant, or a particular pattern of splicing variants.

Both such alterations (quantitative and qualitative) can also be present. An “alteration” in the polypeptide expression or composition, refers to an alteration in expression or composition in a test sample, as compared with the expression or composition of polypeptide by a FLAP nucleic acid in a control sample. A control sample is a sample that corresponds to the test sample (e.g., is from the same type of cells), and is from an individual who is not affected by the disease or a susceptibility to a disease or condition associated with a FLAP nucleic acid. An alteration in the expression or composition of the polypeptide in the test sample, as compared with the control sample, is indicative of a susceptibility to a disease or condition associated with FLAP (e.g., MI, ACS, stroke or PAOD). Similarly, the presence of one or more different splicing variants in the test sample, or the presence of significantly different amounts of different splicing variants in the test sample, as compared with the control sample, is indicative of a susceptibility to a disease or condition associated with a FLAP nucleic acid. Various means of examining expression or composition of the polypeptide encoded by a FLAP nucleic acid can be used, including: spectroscopy, colorimetry, electrophoresis, isoelectric focusing and immunoassays (e.g., David et al., U.S. Pat. No. 4,376,110) such as immunoblotting (see also Current Protocols in Molecular Biology, particularly Chapter 10). For example, in one embodiment, an antibody capable of binding to the polypeptide (e.g., as described above), preferably an antibody with a detectable label, can be used. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by an altered FLAP (e.g., by a FLAP having a SNP as shown in Table 3), or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered FLAP, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a susceptibility to a disease or condition associated with FLAP, as is the presence (or absence) of particular splicing variants encoded by the FLAP nucleic acid.

In one embodiment of this method, the level or amount of polypeptide encoded by a FLAP nucleic acid in a test sample is compared with the level or amount of the polypeptide encoded by the FLAP in a control sample. A level or amount of the polypeptide in the test sample that is higher or lower than the level or amount of the polypeptide in the control sample, such that the difference is statistically significant, is indicative of an alteration in the expression of the polypeptide encoded by the FLAP, and is diagnostic for a susceptibility to a disease or condition associated with that FLAP. Alternatively, the composition of the polypeptide encoded by a FLAP nucleic acid in a test sample is compared with the composition of the polypeptide encoded by the FLAP in a control sample (e.g., the presence of different splicing variants). A difference in the composition of the polypeptide in the test sample, as compared with the composition of the polypeptide in the control sample, is diagnostic for a susceptibility to a disease or condition associated with that FLAP. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to a disease or condition associated with FLAP (e.g., MI).

The invention further pertains to a method for the diagnosis and identification of susceptibility to myocardial infarction, ACS, stroke or PAOD in an individual, by identifying an at-risk haplotype in FLAP. In one embodiment, the at-risk haplotype is one which confers a significant risk of MI, ACS, stroke or PAOD. In one embodiment, significance associated with a haplotype is measured by an odds ratio. In a further embodiment, the significance is measured by a percentage. In one embodiment, a significant risk is measured as an odds ratio of at least about 1.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95, and 98%. In a further embodiment, a significant increase in risk is at least about 50%. It is understood however, that identifying whether a risk is medically significant may also depend on a variety of factors, including the specific disease, the haplotype, and often, environmental factors.

The invention also pertains to methods of diagnosing a susceptibility to myocardial infarction, ACS, stroke or PAOD in an individual, comprising screening for an at-risk haplotype in the FLAP nucleic acid that is more frequently present in an individual susceptible to myocardial infarction (affected), compared to the frequency of its presence in a healthy individual (control), wherein the presence of the haplotype is indicative of susceptibility to myocardial infarction. Standard techniques for genotyping for the presence of SNPs and/or microsatellite markers that are associated with myocardial infarction, ACS, stroke or PAOD can be used, such as fluorescent based techniques (Chen, et al., Genome Res. 9, 492 (1999), PCR, LCR, Nested PCR and other techniques for nucleic acid amplification. In a preferred embodiment, the method comprises assessing in an individual the presence or frequency of SNPs and/or microsatellites in the FLAP nucleic acid that are associated with myocardial infarction, ACS, stroke or PAOD, wherein an excess or higher frequency of the SNPs and/or microsatellites compared to a healthy control individual is indicative that the individual is susceptible to myocardial infarction, ACS, stroke or PAOD. See table 9for SNPs that comprise haplotypes that can be used as screening tools. See also Table 3 that sets forth SNPs and markers for use as screening tools.

In one embodiment, the at-risk haplotype is characterized by the presence of polymorphism(s) represented in Table 3. For example, DG00AAFIU, where the SNP can be a “C” or a “T”; SG13S25, where the SNP can be a “G” or an “A”; DG00AAJFF, where the SNP can be a “G” or an “A”; DG00AAHII, where the SNP can be a “G” or an “A”; DG00AAHID, where the SNP can be a “T” or an “A”; B_SNP_{—}310657, where the SNP can be a “G” or an “A”; SG13S30, where the SNP can be a “G” or a “T”; SG13S32, where the SNP can be a “C” or an “A”; SG13S42, where the SNP can be a “G” or an “A”; and SG13S35, where the SNP can be a “G” or an “A”.

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies which bind to altered or to non-altered (native) FLAP polypeptide, means for amplification of nucleic acids comprising a FLAP, or means for analyzing the nucleic acid sequence of a nucleic acid described herein, or for analyzing the amino acid sequence of a polypeptide as described herein, etc. In one embodiment, a kit for diagnosing susceptibility to MI, ACS, stroke or PAOD can comprise primers for nucleic acid amplification of a region in the FLAP nucleic acid comprising an at-risk haplotype that is more frequently present in an individual having MI, ACS, stroke or PAOD or susceptible to MI, ACS, stroke or PAOD. The primers can be designed using portions of the nucleic acids flanking SNPs that are indicative of MI. In a particularly preferred embodiment, the primers are designed to amplify regions of the FLAP nucleic acid associated with an at-risk haplotype for MI, ACS, stroke or PAOD, as shown in Table 9, or more particularly the haplotype defined by the following SNP markers: In one embodiment, a haplotype associated with a susceptibility to myocardial infarction, ACS, stroke or PAOD comprises markers DG00AAFIU, SG13S25, DG00AAJFF, DG00AAHII, SG13S32 and SG13S35 at the 13q12 locus. In one particular embodiment, the presence of the alleles T, G, G, G, A and G at DG00AAFIU, SG13S25, DG00AAJFF, DG00AAHII, SG13S32 and SG13S35, respectively (the B6 haplotype), is diagnostic of susceptibility to myocardial infarction, ACS, stroke or PAOD. In another embodiment, a haplotype associated with a susceptibility to myocardial infarction, ACS, stroke or PAOD comprises markers DG00AAFIU, SG13S25, DG00AAHII, SG13S30 and SG13S42 at the 13q12 locus. In one particular embodiment, the presence of the alleles T, G, G, G and A at DG00AAFIU, SG13S25, DG00AAHII, SG13S30 and SG13S42, respectively (the B5 haplotype), is diagnostic of susceptibility to myocardial infarction, ACS, stroke or PAOD. In a third embodiment, a haplotype associated with a susceptibility to myocardial infarction, ACS, stroke or PAOD comprises markers SG13S25, DG00AAHII, SG13S30 and SG13S42 at the 13q12 locus. In one particular embodiment, the presence of the alleles G, G, G and A at SG13S25, DG00AAHII, SG13S30 and SG13S42, respectively (the B4 haplotype), is diagnostic of susceptibility to myocardial infarction, ACS, stroke or PAOD. In a fourth embodiment, a haplotype associated with a susceptibility to myocardial infarction, ACS, stroke or PAOD comprises markers DG00AAFIU, SG13S25, DG00AAHID, B_SNP_{—}310657 and SG13S32 at the 13q12 locus. In one particular embodiment, the presence of the alleles T, G, T, G and A at DG00AAFIU, SG13S25, DG00AAHID, B_SNP_{—}310657 and SG13S32, respectively (the A5 haplotype), is diagnostic of susceptibility to myocardial infarction, ACS, stroke or PAOD. In a fifth embodiment, a haplotype associated with a susceptibility to myocardial infarction, ACS, stroke or PAOD comprises markers SG13S25, DG00AAHID, B_SNP_{—}310657 and SG13S32 at the 13q12 locus. In one particular embodiment, the presence of the alleles G, T, G and A at SG13S25, DG00AAHID, B_SNP_{—}310657 and SG13S32, respectively (the A4 haplotype), is diagnostic of susceptibility to myocardial infarction, ACS, stroke or PAOD.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of one of SEQ ID NOs: 1 or 3 or the complement thereof, or a nucleic acid encoding an amino acid having the sequence of SEQ ID NO: 2, or a fragment or variant of such nucleic acids), under stringent conditions as described above, and then assessing the sample for the presence (or absence) of hybridization. In a preferred embodiment, high stringency conditions are conditions appropriate for selective hybridization. In another embodiment, a sample containing a nucleic acid molecule of interest is contacted with a nucleic acid containing a contiguous nucleic acid sequence (e.g., a primer or a probe as described above) that is at least partially complementary to a part of the nucleic acid molecule of interest (e.g., a FLAP nucleic acid), and the contacted sample is assessed for the presence or absence of hybridization. In a preferred embodiment, the nucleic acid containing a contiguous nucleic acid sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes which alter (e.g., increase or decrease) the activity of the polypeptides described herein, or which otherwise interact with the polypeptides herein. For example, such agents can be agents which bind to polypeptides described herein (e.g., binding agent for members of the leukotriene pathway, such as FLAP binding agents); which have a stimulatory or inhibitory effect on, for example, activity of polypeptides of the invention; or which change (e.g., enhance or inhibit) the ability of the polypeptides of the invention to interact with members of the leukotriene pathway binding agents (e.g., receptors or other binding agents); or which alter posttranslational processing of the leukotriene pathway member polypeptide, such as a FLAP polypeptide (e.g., agents that alter proteolytic processing to direct the polypeptide from where it is normally synthesized to another location in the cell, such as the cell surface; agents that alter proteolytic processing such that more polypeptide is released from the cell, etc.)

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Lam, K. S., Anticancer Drug Des. 12:145 (1997)).

In one embodiment, to identify agents which alter the activity of a FLAP polypeptide, a cell, cell lysate, or solution containing or expressing a FLAP polypeptide (e.g., SEQ ID NO: 2 or another splicing variant encoded by a FLAP nucleic acid, such as a nucleic acid comprising a SNP as shown in Table 3), or a fragment or derivative thereof (as described above), can be contacted with an agent to be tested; alternatively, the polypeptide can be contacted directly with the agent to be tested. The level (amount) of FLAP activity is assessed (e.g., the level (amount) of FLAP activity is measured, either directly or indirectly), and is compared with the level of activity in a control (i.e., the level of activity of the FLAP polypeptide or active fragment or derivative thereof in the absence of the agent to be tested). If the level of the activity in the presence of the agent differs, by an amount that is statistically significant, from the level of the activity in the absence of the agent, then the agent is an agent that alters the activity of a FLAP polypeptide. An increase in the level of FLAP activity in the presence of the agent relative to the activity in the absence of the agent, indicates that the agent is an agent that enhances FLAP activity. Similarly, a decrease in the level of FLAP activity in the presence of the agent, relative to the activity in the absence of the agent, indicates that the agent is an agent that inhibits FLAP activity. In another embodiment, the level of activity of a FLAP polypeptide or derivative or fragment thereof in the presence of the agent to be tested, is compared with a control level that has previously been established. A statistically significant difference in the level of the activity in the presence of the agent from the control level indicates that the agent alters FLAP activity.

The present invention also relates to an assay for identifying agents which alter the expression of a FLAP nucleic acid (e.g., antisense nucleic acids, fusion proteins, polypeptides, peptidomimetics, prodrugs, receptors, binding agents, antibodies, small molecules or other drugs, or ribozymes; which alter (e.g., increase or decrease) expression (e.g., transcription or translation) of the nucleic acid or which otherwise interact with the nucleic acids described herein, as well as agents identifiable by the assays. For example, a solution containing a nucleic acid encoding a FLAP polypeptide (e.g., a FLAP nucleic acid) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of FLAP expression (e.g., the level and/or pattern of mRNA or of protein expressed, such as the level and/or pattern of different splicing variants) is assessed, and is compared with the level and/or pattern of expression in a control (i.e., the level and/or pattern of the FLAP expression in the absence of the agent to be tested). If the level and/or pattern in the presence of the agent differ, by an amount or in a manner that is statistically significant, from the level and/or pattern in the absence of the agent, then the agent is an agent that alters the expression of the FLAP nucleic acid. Enhancement of FLAP expression indicates that the agent is an activator of FLAP activity. Similarly, inhibition of FLAP expression indicates that the agent is a repressor of FLAP activity.

In another embodiment, the level and/or pattern of FLAP polypeptide(s) (e.g., different splicing variants) in the presence of the agent to be tested, is compared with a control level and/or pattern that have previously been established. A level and/or pattern in the presence of the agent that differs from the control level and/or pattern by an amount or in a manner that is statistically significant indicates that the agent alters FLAP expression.

In another embodiment of the invention, agents which alter the expression of a FLAP nucleic acid or which otherwise interact with the nucleic acids described herein, can be identified using a cell, cell lysate, or solution containing a nucleic acid encoding the promoter region of the FLAP nucleic acid operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of the FLAP nucleic acid, as indicated by its ability to alter expression of a nucleic acid that is operably linked to the FLAP nucleic acid promoter.

Enhancement of the expression of the reporter indicates that the agent is an activator of FLAPexpression. Similarly, inhibition of the expression of the reporter indicates that the agent is a repressor of FLAPexpression. In another embodiment, the level of expression of the reporter in the presence of the test agent, is compared with a control level that has previously been established. A level in the presence of the agent that differs from the control level by an amount or in a manner that is statistically significant indicates that the agent alters expression.

Agents which alter the amounts of different splicing variants encoded by a FLAP nucleic acid (e.g., an agent which enhances expression of a first splicing variant, and which inhibits expression of a second splicing variant), as well as agents which stimulate activity of a first splicing variant and inhibit activity of a second splicing variant, can easily be identified using these methods described above.

In other embodiments of the invention, assays can be used to assess the impact of a test agent on the activity of a polypeptide relative to a FLAP binding agent. For example, a cell that expresses a compound that interacts with a FLAP nucleic acid (herein referred to as a “FLAP binding agent”, which can be a polypeptide or other molecule that interacts with a FLAP nucleic acid, such as a receptor, or another molecule, such as 5-LO) is contacted with a FLAP in the presence of a test agent, and the ability of the test agent to alter the interaction between the FLAP and the FLAP binding agent is determined. Alternatively, a cell lysate or a solution containing the FLAP binding agent, can be used. An agent which binds to the FLAP or the FLAP binding agent can alter the interaction by interfering with, or enhancing the ability of the FLAP to bind to, associate with, or otherwise interact with the FLAP binding agent. Determining the ability of the test agent to bind to a FLAP nucleic acid or a FLAP nucleic acid binding agent can be accomplished, for example, by coupling the test agent with a radioisotope or enzymatic label such that binding of the test agent to the polypeptide can be determined by detecting the labeled with ¹²⁵I, ³⁵S, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with a FLAP or a FLAP binding agent without the labeling of either the test agent, FLAP, or the FLAP binding agent. McConnell, H. M. et al., Science 257:1906-1912 (1992). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide.

Thus, these receptors can be used to screen for compounds that are agonists for use in treating a disease or condition associated with FLAP or a susceptibility to a disease or condition associated with FLAP, or antagonists for studying a susceptibility to a disease or condition associated with FLAP (e.g., MI, ACS, stroke or PAOD). Drugs can be designed to regulate FLAP activation, that in turn can be used to regulate signaling pathways and transcription events of genes downstream or of proteins or polypeptides interacting with FLAP (e.g., 5-LO).

In another embodiment of the invention, assays can be used to identify polypeptides that interact with one or more FLAP polypeptides, as described herein. For example, a yeast two-hybrid system such as that described by Fields and Song (Fields, S. and Song, O., Nature 340:245-246 (1989)) can be used to identify polypeptides that interact with one or more FLAP polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used which includes a nucleic acid encoding a DNA binding domain and also a FLAP polypeptide, splicing variant, or fragment or derivative thereof, and a second vector is used which includes a nucleic acid encoding a transcription activation domain and also a nucleic acid encoding a polypeptide which potentially may interact with the FLAP polypeptide, splicing variant, or fragment or derivative thereof (e.g., a FLAP polypeptide binding agent or receptor). Incubation of yeast containing the first vector and the second vector under appropriate conditions (e.g., mating conditions such as used in the Matchmaker™ system from Clontech (Palo Alto, Calif., USA)) allows identification of colonies that express the markers of interest. These colonies can be examined to identify the polypeptide(s) that interact with the FLAP polypeptide or fragment or derivative thereof. Such polypeptides may be useful as agents that alter the activity of expression of a FLAP polypeptide, as described above.

In more than one embodiment of the above assay methods of the present invention, it may be desirable to immobilize either the FLAP, the FLAP binding agent, or other components of the assay on a solid support, in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided which adds a domain that allows a FLAP nucleic acid or a FLAP binding agent to be bound to a matrix or other solid support.

In another embodiment, modulators of expression of nucleic acid molecules of the invention are identified in a method wherein a cell, cell lysate, or solution containing a nucleic acid encoding a FLAP nucleic acid is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

In yet another embodiment, the invention provides methods for identifying agents (e.g., fusion proteins, polypeptides, peptidomimetics, prodrugs, receptors, binding agents, antibodies, small molecules or other drugs, or ribozymes) which alter (e.g., increase or decrease) the activity of a member of leukotriene pathway binding agent, such as a FLAP binding agent (e.g., 5-LO), as described herein. For example, such agents can be agents which have a stimulatory or inhibitory effect on, for example, the activity of a member of leukotriene pathway binding agent, such as a FLAP binding agent; which change (e.g., enhance or inhibit) the ability a member of leukotriene pathway binding agents, (e.g., receptors or other binding agents) to interact with the polypeptides of the invention; or which alter posttranslational processing of the member of leukotriene pathway binding agent, (e.g., agents that alter proteolytic processing to direct the member of the leukotriene pathway binding agent from where it is normally synthesized to another location in the cell, such as the cell surface; agents that alter proteolytic processing such that more active binding agent is released from the cell, etc.).

For example, the invention provides assays for screening candidate or test agents that bind to or modulate the activity of a member of the leukotriene pathway (or enzymatically active portion(s) thereof), as well as agents identifiable by the assays. As described above, test agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small-molecule libraries of compounds (Lam, K. S. Anticancer Drug Des., 12:145 (1997)).

In one embodiment, to identify agents which alter the activity of a member of the leukotriene pathway (such as a FLAP binding agent, or an agent which binds to a member of the leukotriene pathway (a “binding agent”)), a cell, cell lysate, or solution containing or expressing a binding agent (e.g., 5-LO, or a leukotriene pathway member receptor, or other binding agent), or a fragment (e.g., an enzymatically active fragment) or derivative thereof, can be contacted with an agent to be tested; alternatively, the binding agent (or fragment or derivative thereof) can be contacted directly with the agent to be tested. The level (amount) of binding agent activity is assessed (either directly or indirectly), and is compared with the level of activity in a control (i.e., the level of activity in the absence of the agent to be tested). If the level of the activity in the presence of the agent differs, by an amount that is statistically significant, from the level of the activity in the absence of the agent, then the agent is an agent that alters the activity of the member of the leukotriene pathway. An increase in the level of the activity relative to a control, indicates that the agent is an agent that enhances (is an agonist of) the activity. Similarly, a decrease in the level of activity relative to a control, indicates that the agent is an agent that inhibits (is an antagonist of) the activity. In another embodiment, the level of activity in the presence of the agent to be tested, is compared with a control level that has previously been established. A level of the activity in the presence of the agent that differs from the control level by an amount that is statistically significant indicates that the agent alters the activity.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent.

Furthermore, this invention pertains to uses of novel agents identified by the above-described screening assays for treatments as described herein. In addition, an agent identified as described herein can be used to alter activity of a polypeptide encoded by a FLAP nucleic acid, or to alter expression of a FLAP nucleic acid, by contacting the polypeptide or the nucleic acid (or contacting a cell comprising the polypeptide or the nucleic acid) with the agent identified as described herein.

The present invention is now illustrated by the following Examples, which are not intended to be limiting in any way. The teachings of all references cited are incorporated herein in their entirety.

### EXAMPLE 1

### Identification of Gene and Haplotypes Associated with MI

Subjects and Methods

Study Population

Patients entering the study were defined from an infarction registry that includes all MIs (over 8,000 patients) in Iceland 1981-2000. This registry is a part of the World Health Organization MONICA Project (The World Health Organization MONICA Project (monitoring trends and determinants in cardiovascular disease): a major international collaboration. (WHO MONICA Project Principal Investigators. J Clin. Epidemiol. 1988; 41:105-14). Diagnosis of all patients in the registry follow strict diagnostic rules based on symptoms, electrocardiograms, cardiac enzymes, and necropsy findings.

Blood samples from 570 female MI patients and 1380 male patients, both cases with a family history and sporadic cases were collected. For each patient that participated, blood was collected from 2 relatives (unaffected or affected). Their genotypes were used to help with construction of haplotypes.

Linkage Analysis

One hundred and sixty female MI patients were clustered into large extended families such that each patient is related to at least one other patient within and including six meiotic events (e.g., 6 meiotic events separate second cousins). The information regarding the relatedness of patients was obtained from an encrypted genealogy database that covers the entire Icelandic nation (Gulcher et al., Eur. J. Hum. Genet. 8: 739-742 (2000)). A genomewide scan was performed using a framework map of 1000 microsatellite markers, using protocols described elsewhere (Gretarsdottir S., et al. Am. J. Hum. Genet.,70: 593-603, 2002)). The marker order and positions were obtained from deCODE genetics' high resolution genetic map (Kong A, et al., Nat. genet., 31: 241-247 (2002)). The population-based allelic frequencies were constructed from a cohort of more than 30,000 Icelanders who have participated in genetic studies of various disease projects. Additional markers were genotyped within the highest linkage peak on chromosome 13 to increase the information on identity by descent within the families. For those markers at least 180 Icelandic controls were genotyped to derive the population allele frequencies.

For statistical analysis, multipoint, affected-only allele-sharing methods were used to assess evidence for linkage. All results, both the LOD and the non-parametric linkage (NPL) score, were obtained using the program ALLEGRO (Gudbjartsson D. F., et al., Nat Genet., 25: 12-13(2000)). The baseline linkage analysis (Gretarsdottir S., et al., Am. J. Hum. Genet. 70: 593-603, (2002)) uses the Spairs scoring function (Whittermore A S, and Haplern J A., Biometrics 50: 118-127 (1994)) and Kruglyak et al., Am. J. Hum. Genet., 58:1347-1363 (1996)) the exponential allele-sharing model (Kong A., and Cox N. J., Am. J. Hum. Genet. 61:1179-1188 (1997)), and a family weighting scheme which is halfway, on the log-scale, between weighing each affected pairs equally and weighing each family equally.

Ultra-Fine Mapping and Haplotype Analysis:

A candidate susceptibility locus was defined as the region under the LOD score curve where the score was one lod lower than the highest lod score. This region (approx. 12 Mb) was ultra-finemapped with microsatellite markers with an average spacing between markers of less than 100 Kb. All usable microsatellite markers found in public databases and mapped within that region were used. In addition, microsatellite markers identified within the deCODE genetics sequence assembly of the human genome were used.

Haplotype Analysis.

The frequencies of haplotypes were estimated in the patient and the control groups using an expectation-maximization algorithm (Dempster A. P. et al., J R. Stat. Soc. B. 39: 1-389 (1977)). An implementation of this algorithm that can handle missing genotypes and uncertainty with the phase was used. Under the null hypothesis, the patients and the controls are assumed to have identical frequencies. Using a likelihood approach, an alternative hypothesis where a candidate at-risk-haplotype is allowed to have a higher frequency in patients than controls, while the ratios of the frequencies of other haplotypes are assumed to be the same in both groups was tested. Likelihoods are maximized separately under both hypothesis and a corresponding 1-df likelihood ratio statistic is used to evaluate the statistic significance.

To look for at-risk-haplotypes in the 1-lod drop, association of all possible combinations of genotyped markers was studied, provided those markers spanned a region of size less than 1000 Kb. Due to a certain amount of testing, the p-values were adjusted using simulations. The combined patient and control groups were randomly divided into two sets, equal in size to the original group of patients and controls. The haplotype analysis was then repeated and the most significant p-value registered was observed. This randomization scheme was repeated over 100 times to construct an empirical distribution of p-values.

Results and Discussion

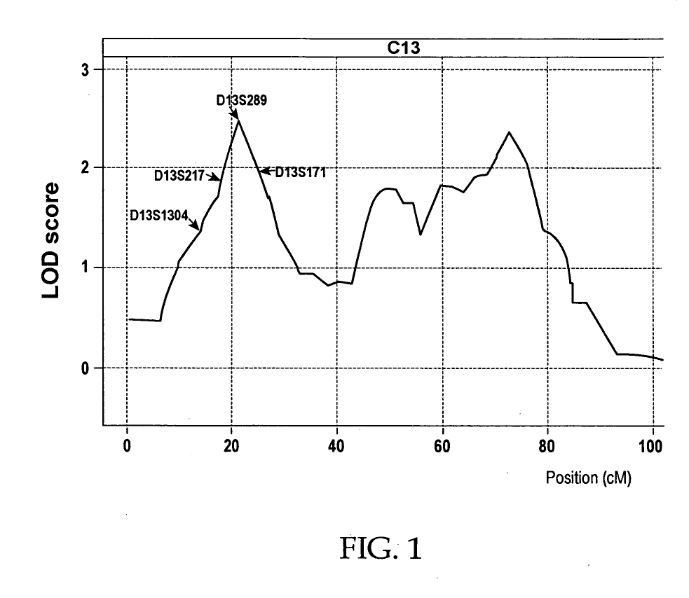

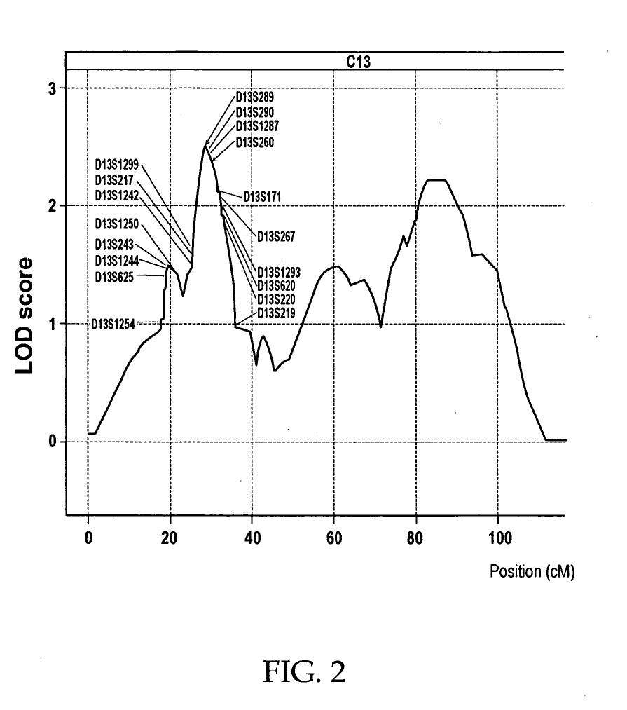

In a genome wide search for susceptibility genes for MI, a locus was mapped to a location on chromosome 13q 12. FIG. 1 shows the multipoint non-parametric LOD scores a linkage scan for a framework marker map on chromosome 13. A LOD score suggestive of linkage of 2.5 was found centered at marker D13S289. The marker map for chromosome 13 that was used in the linkage analysis is shown in Table 1. The LOD score at this location remained with increased number of microsatellite markers which increased information content of the linkage (FIG. 2).

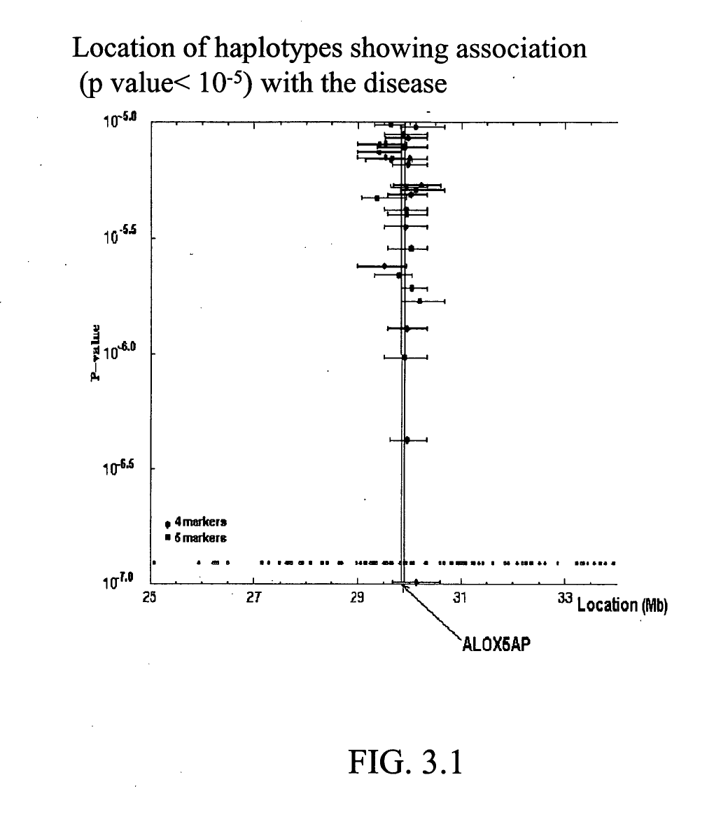

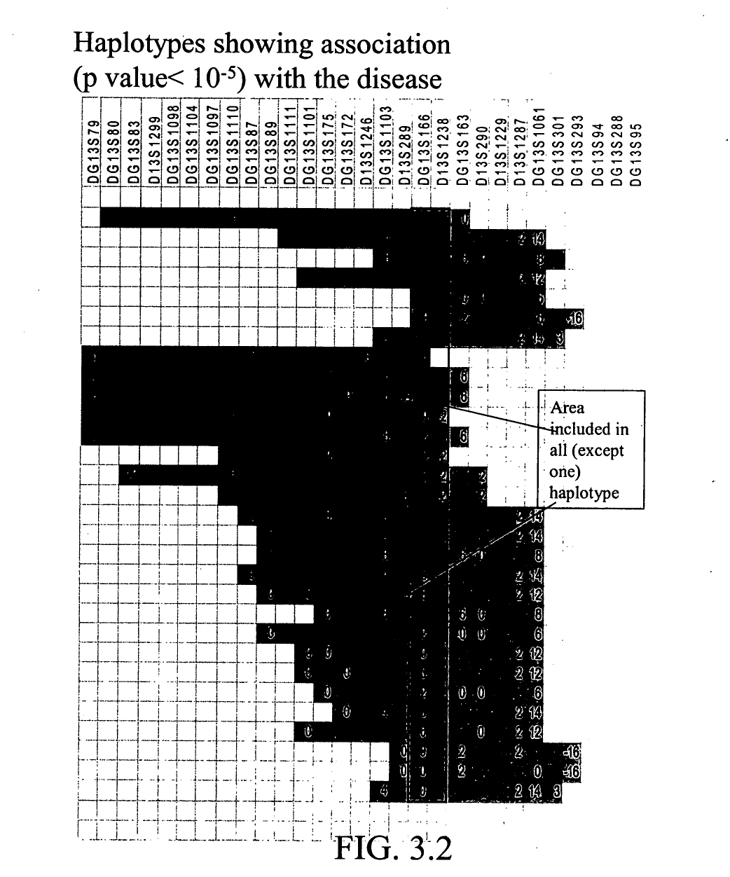

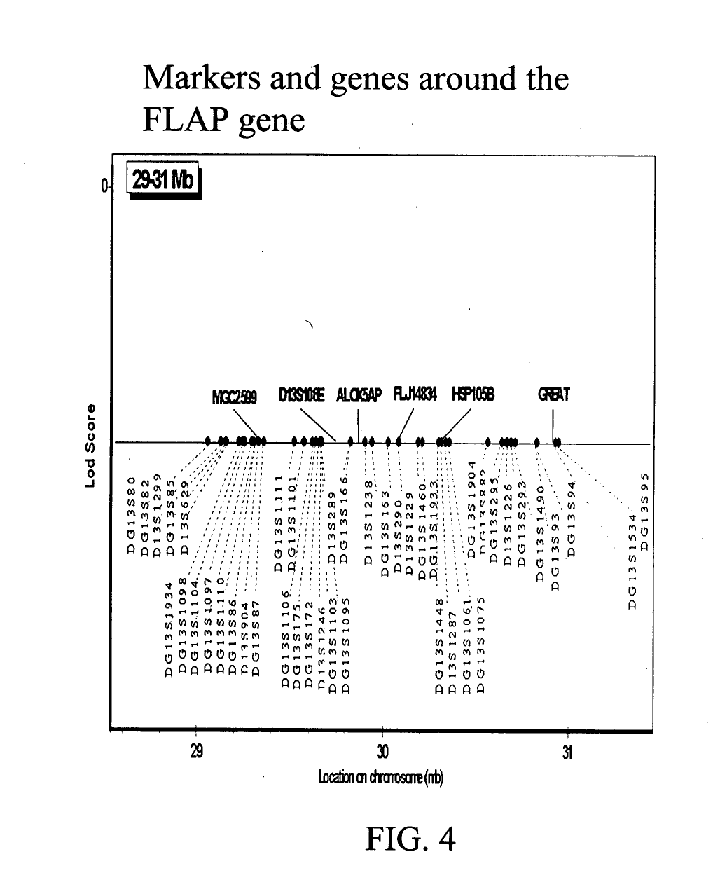

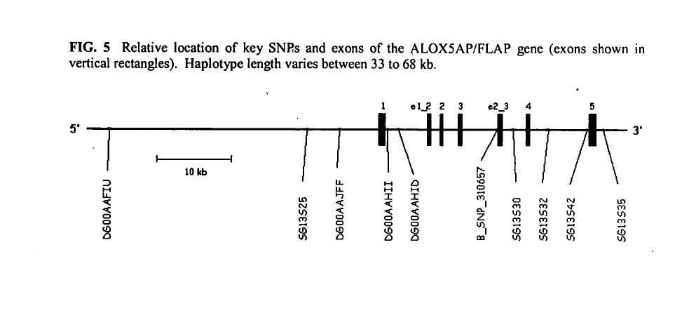

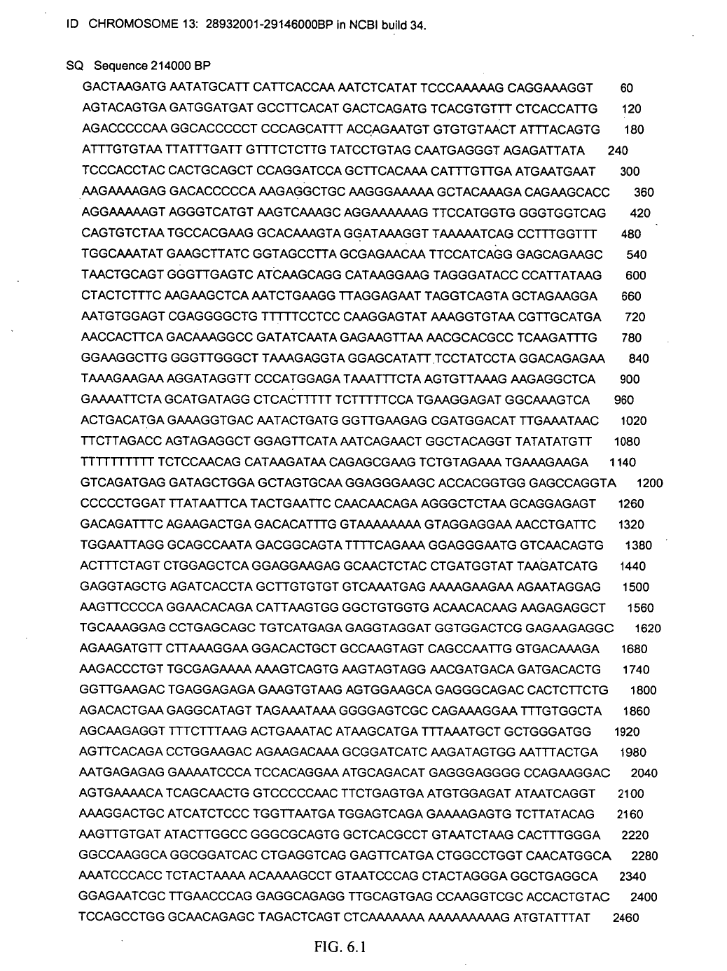

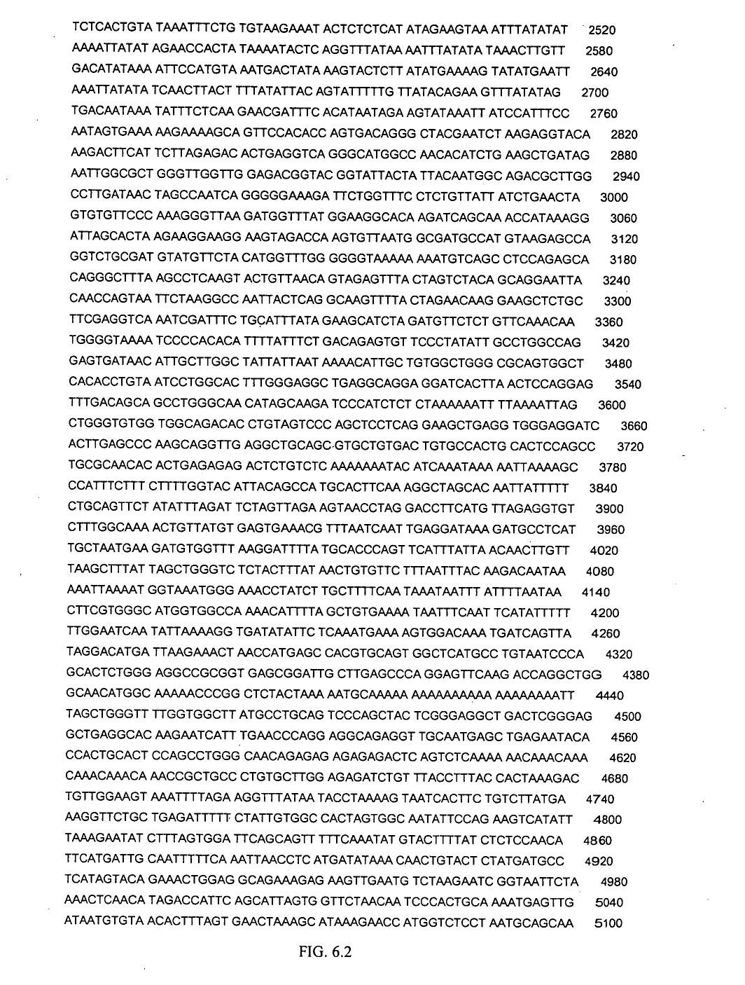

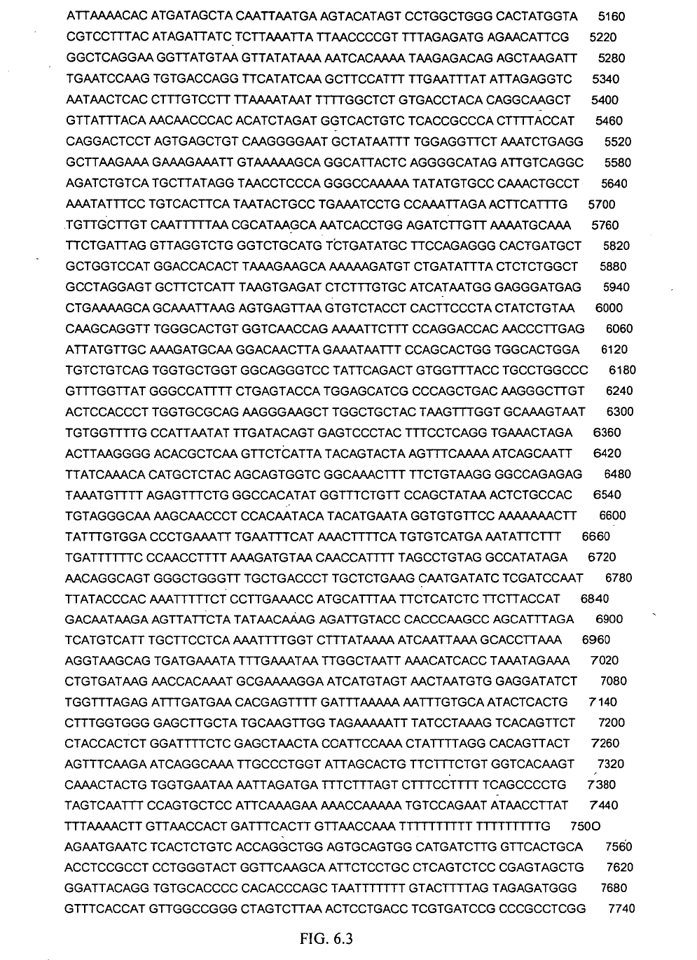

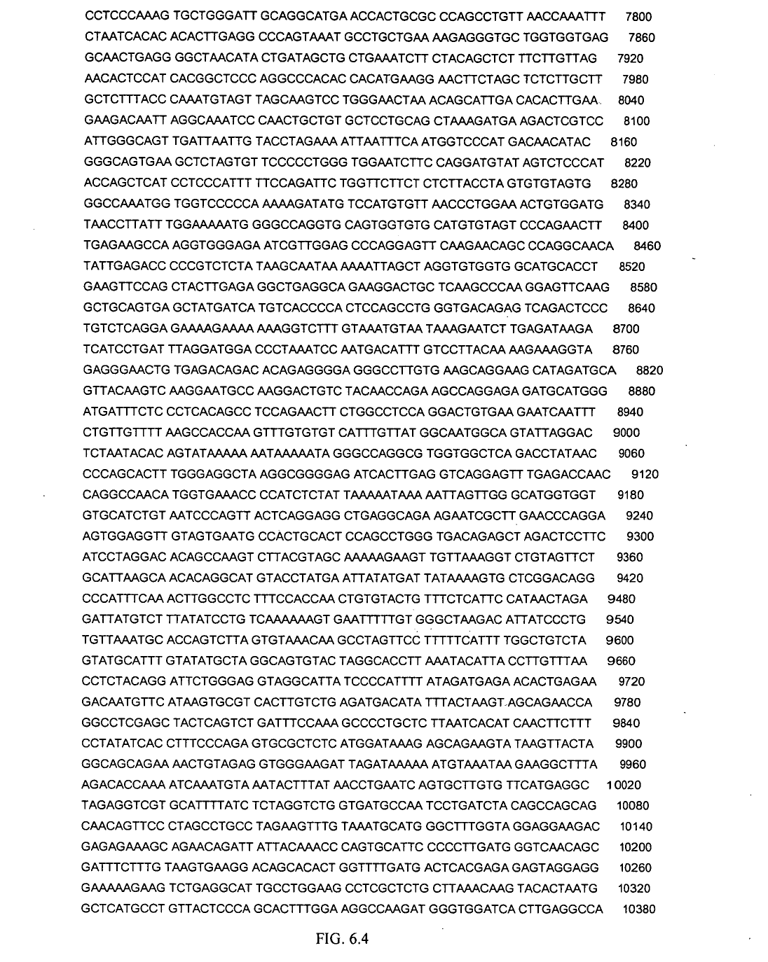

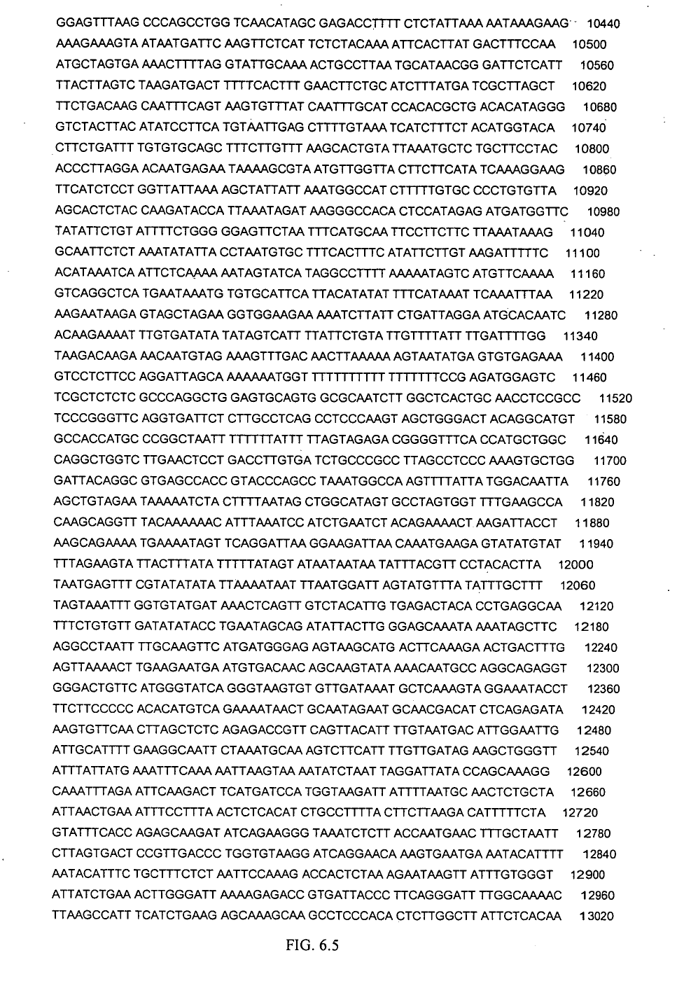

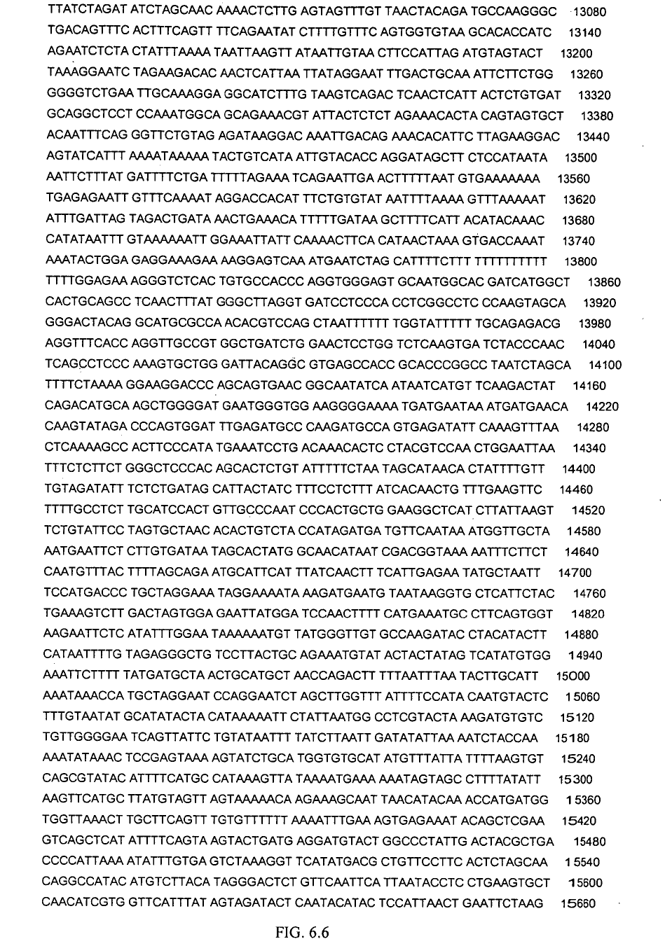

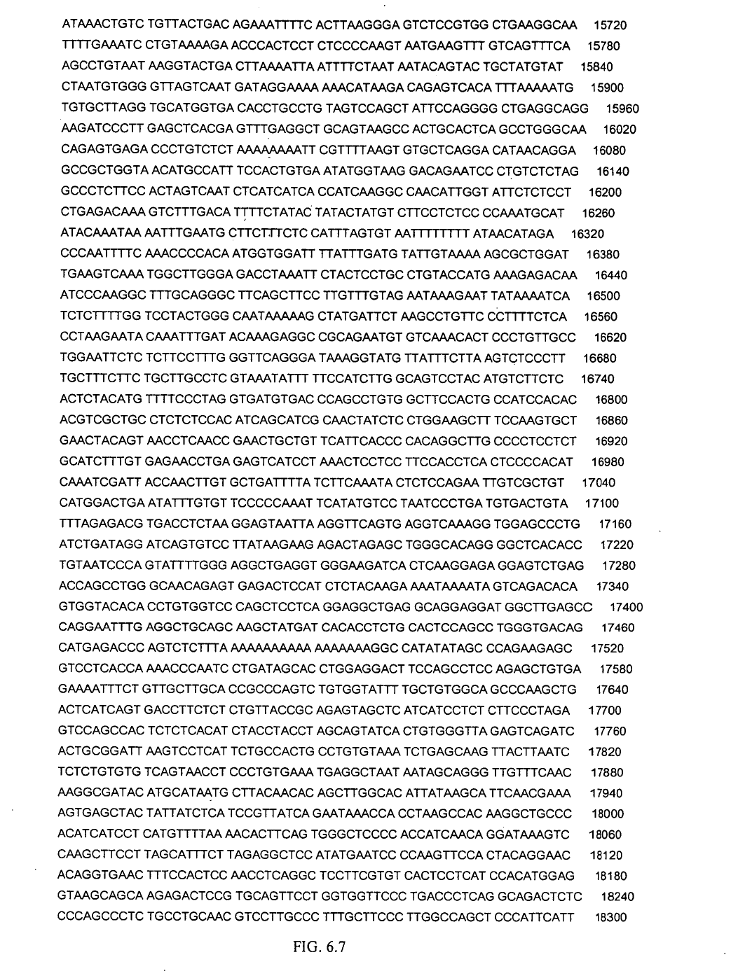

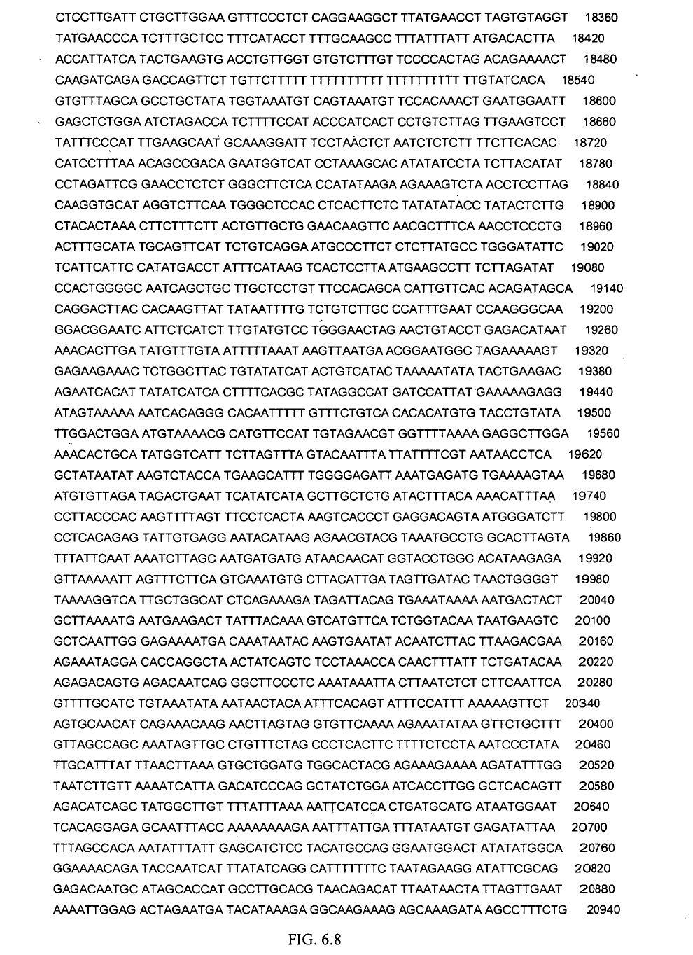

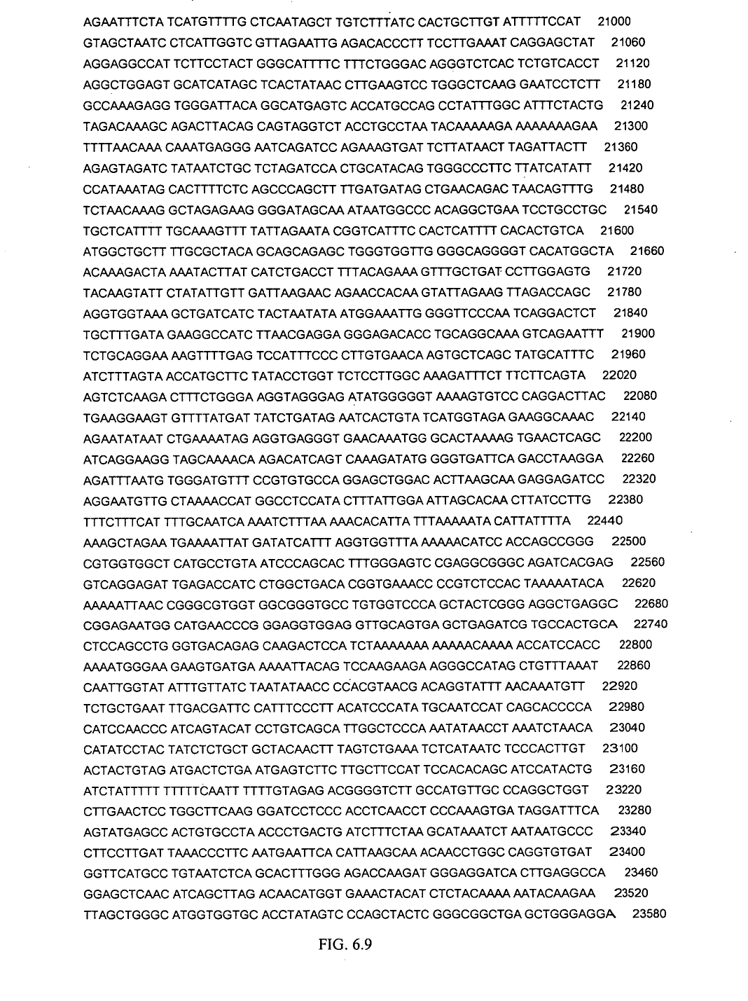

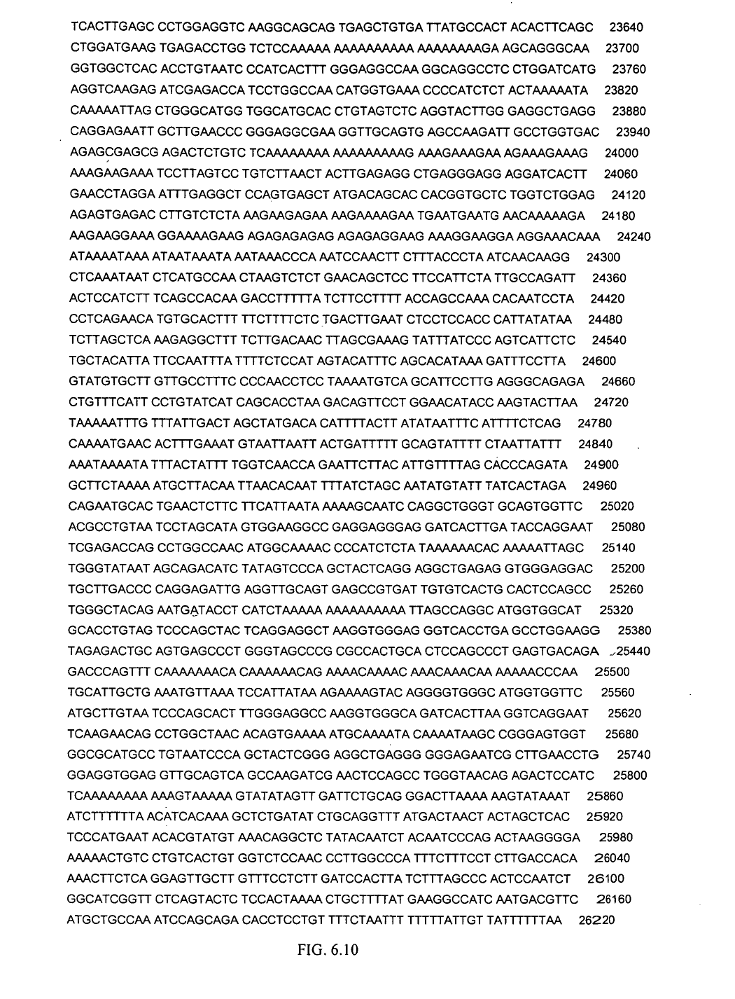

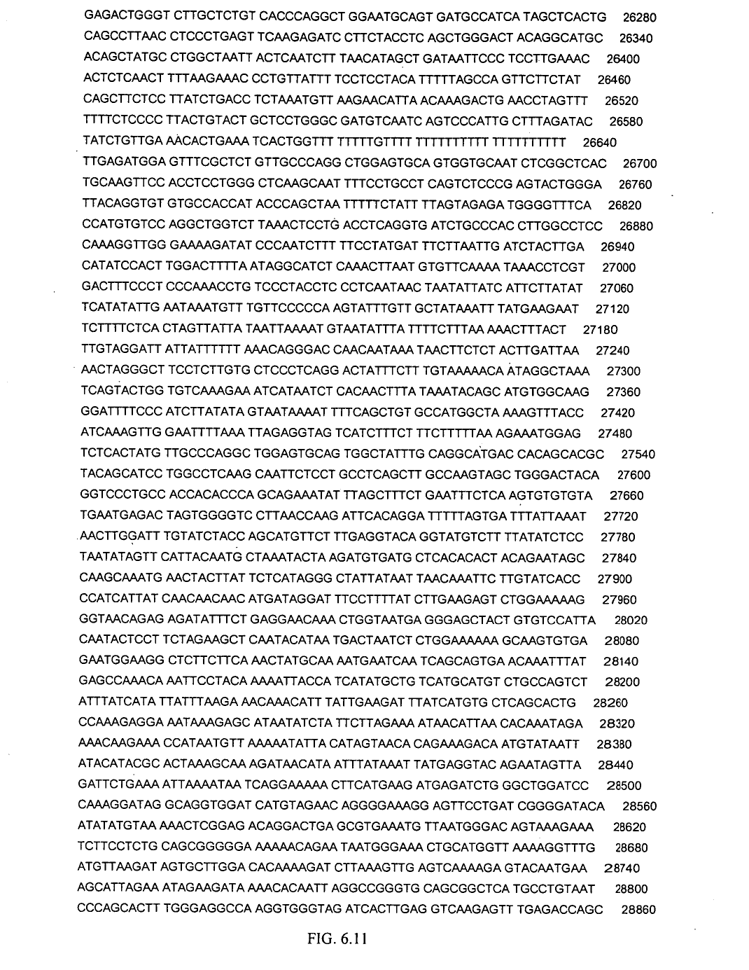

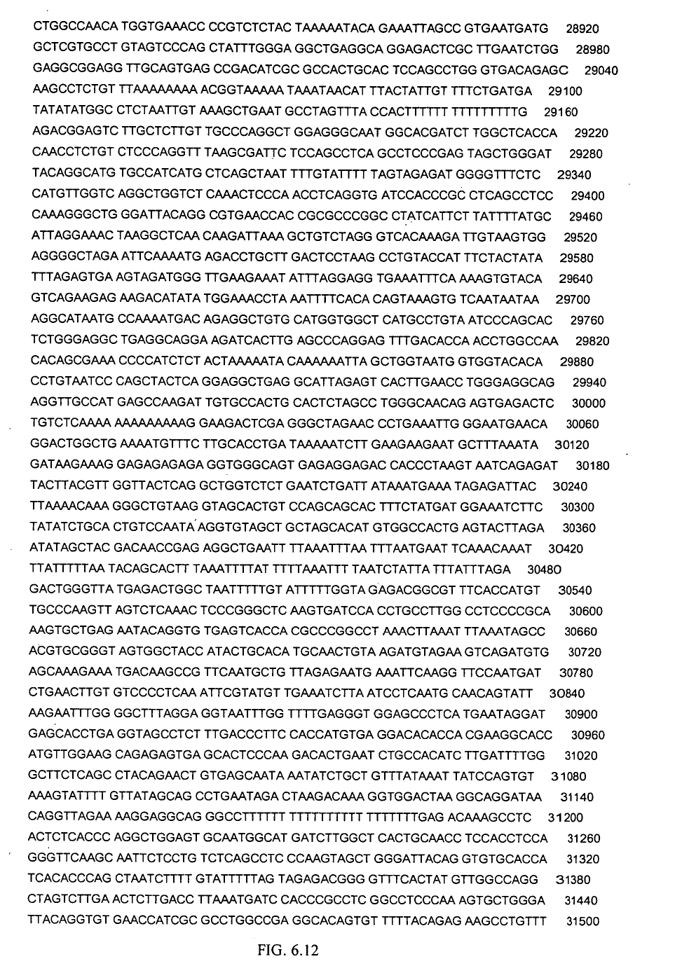

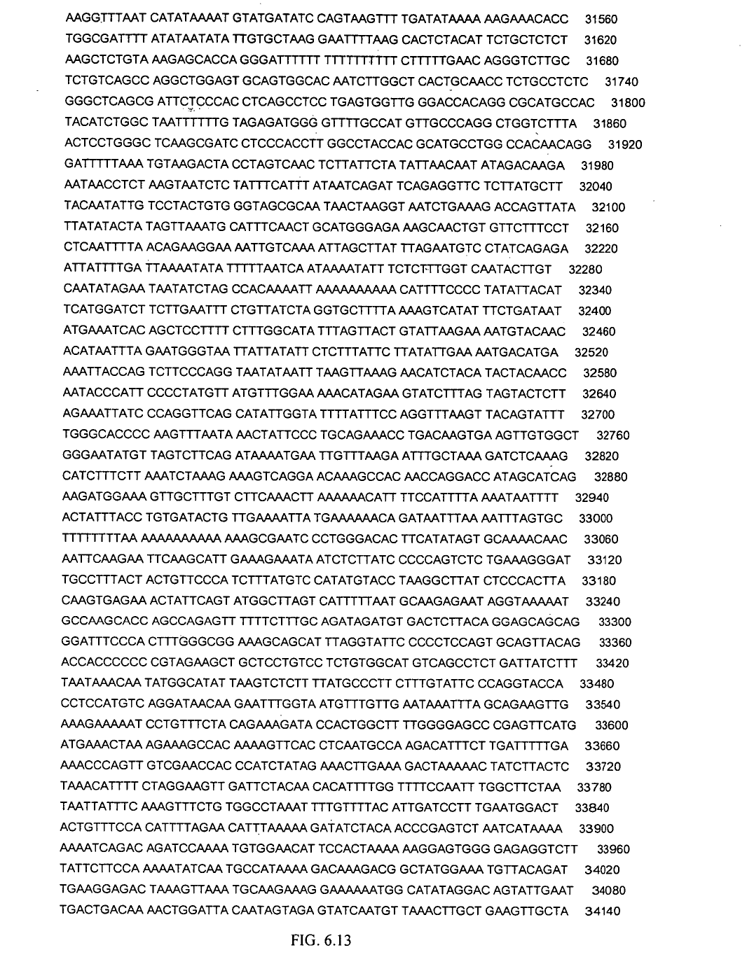

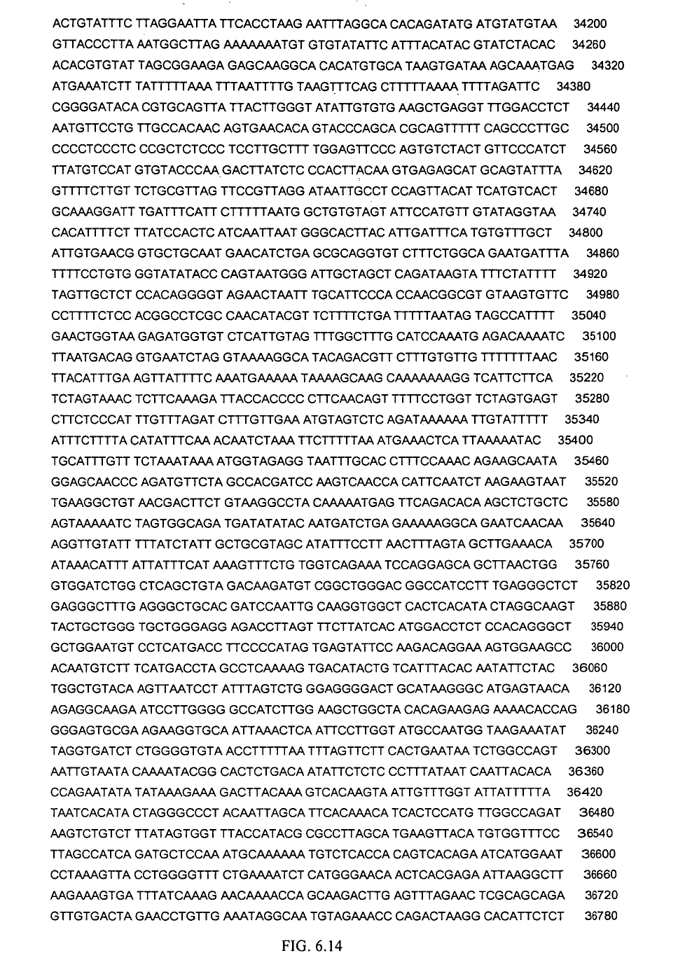

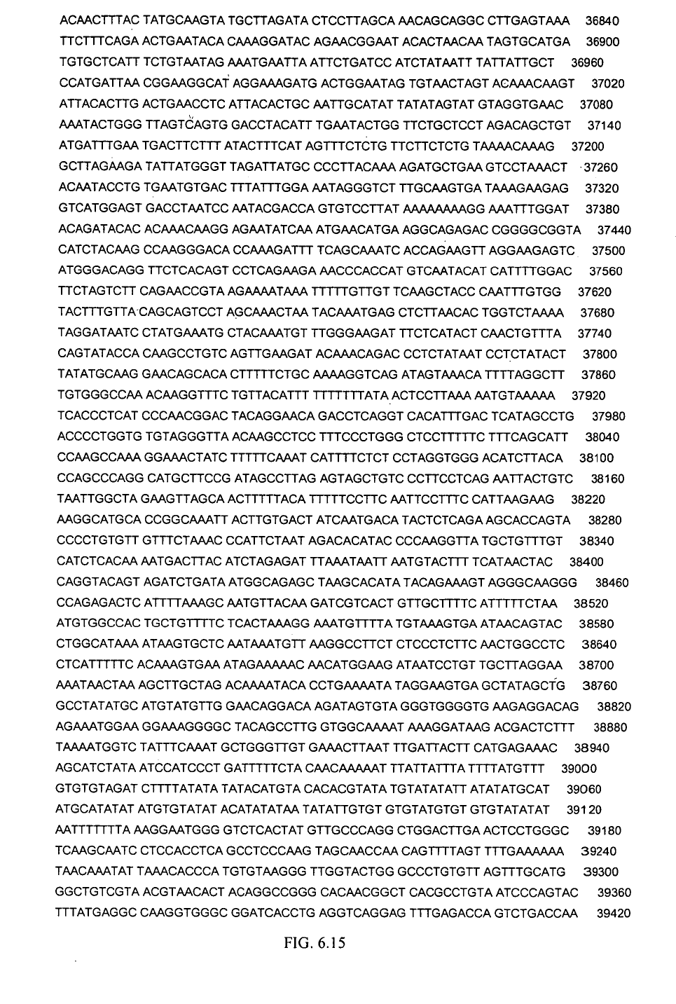

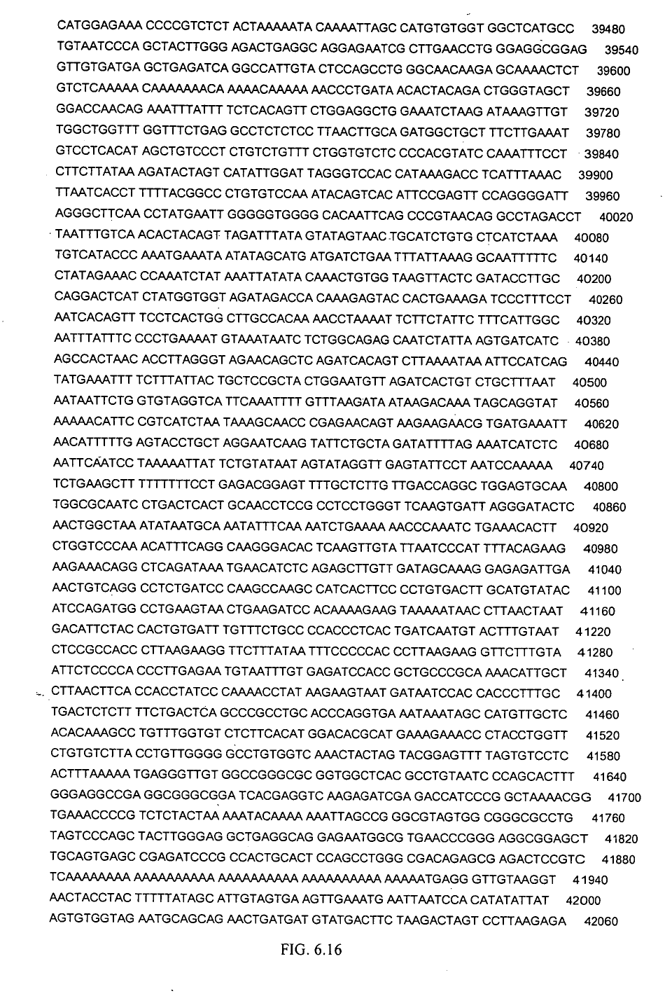

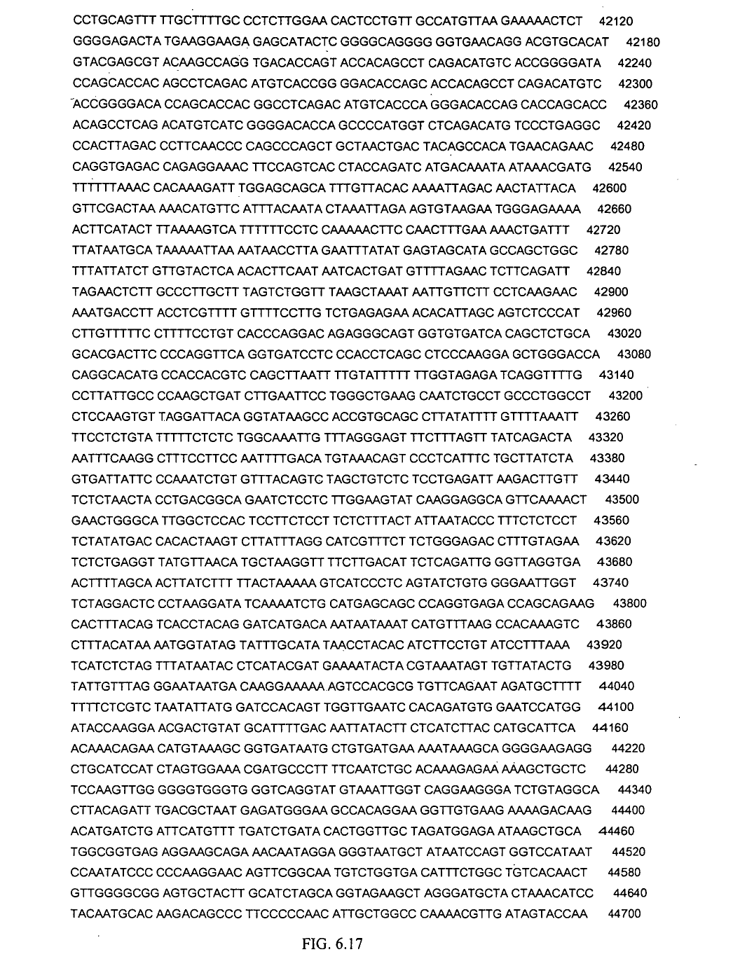

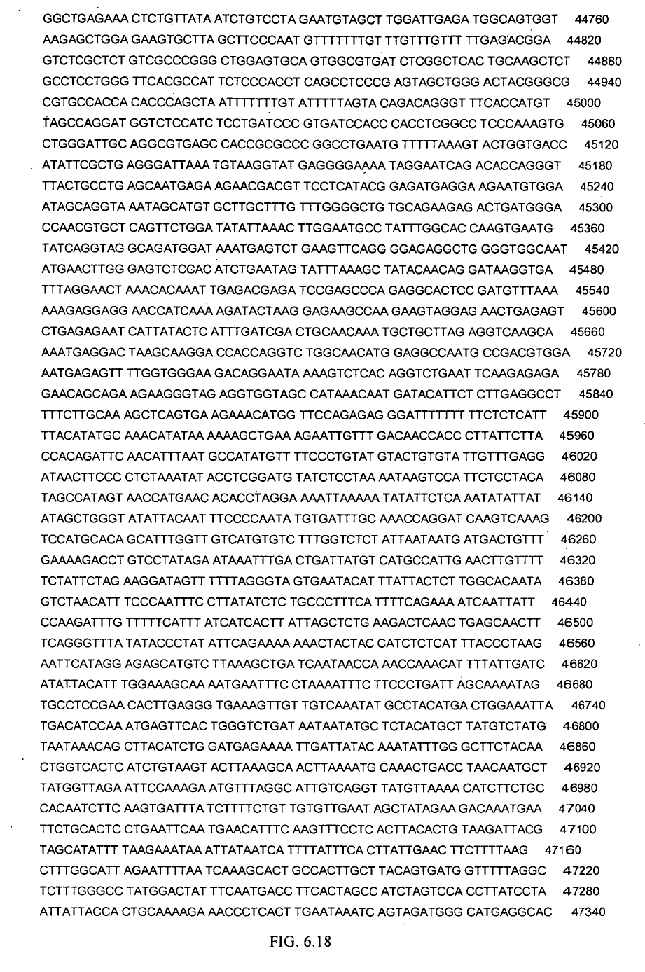

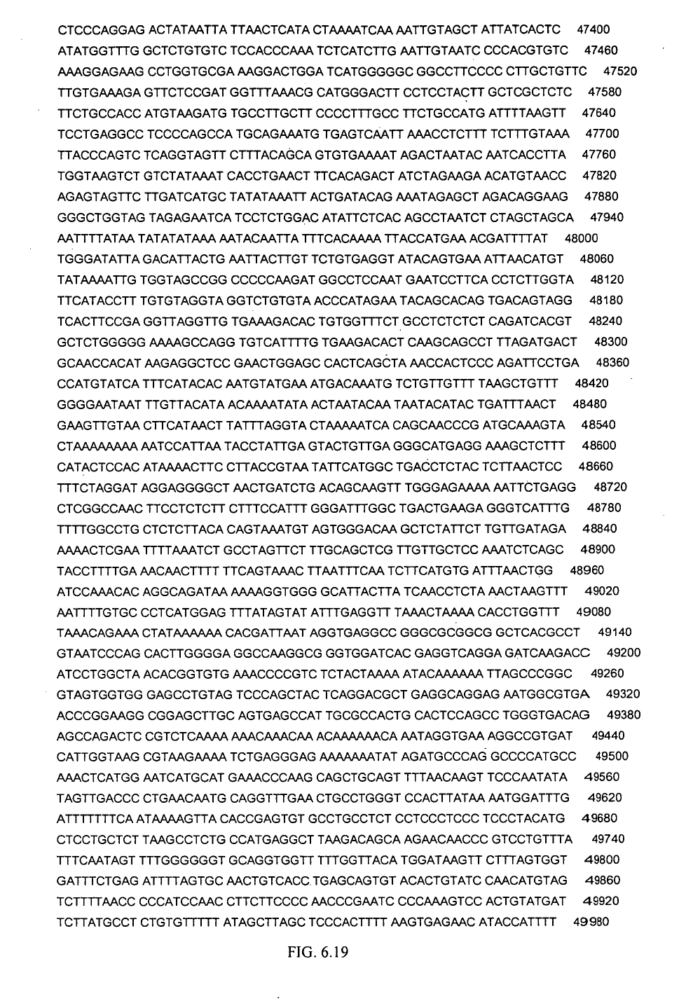

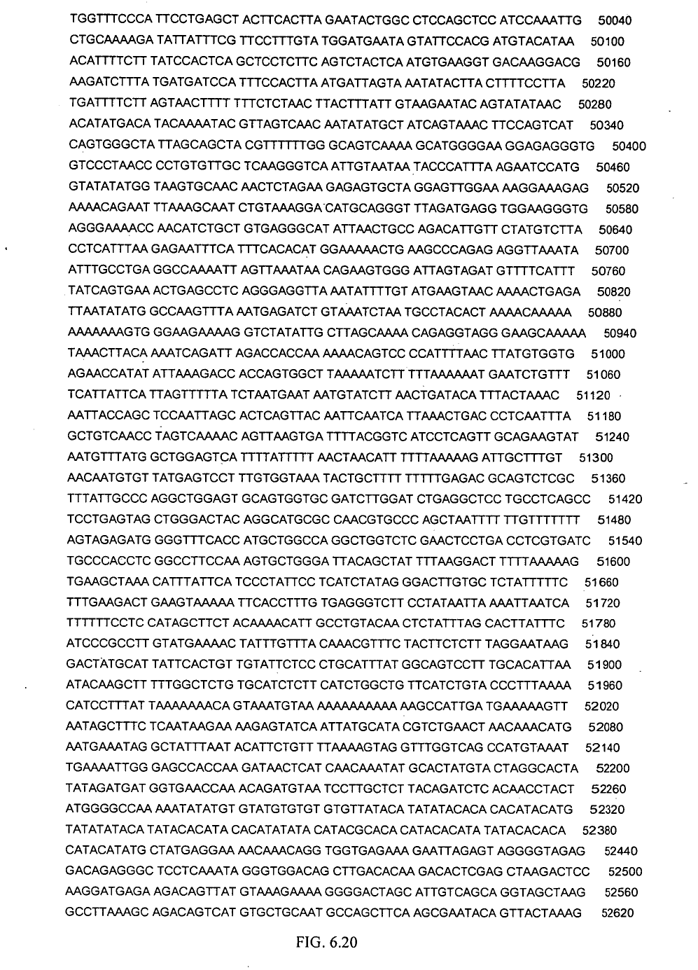

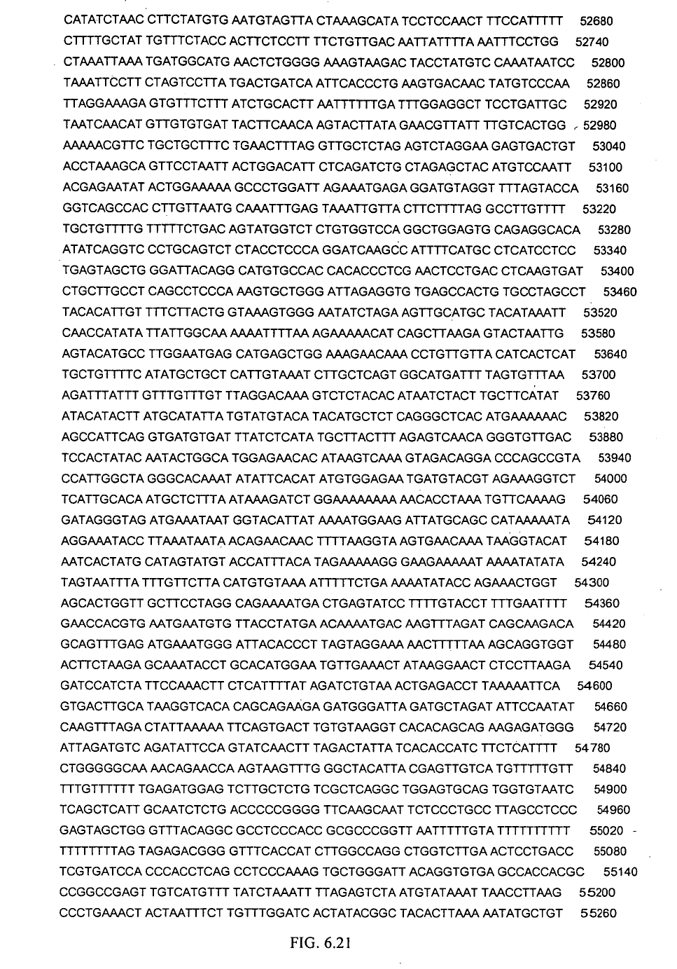

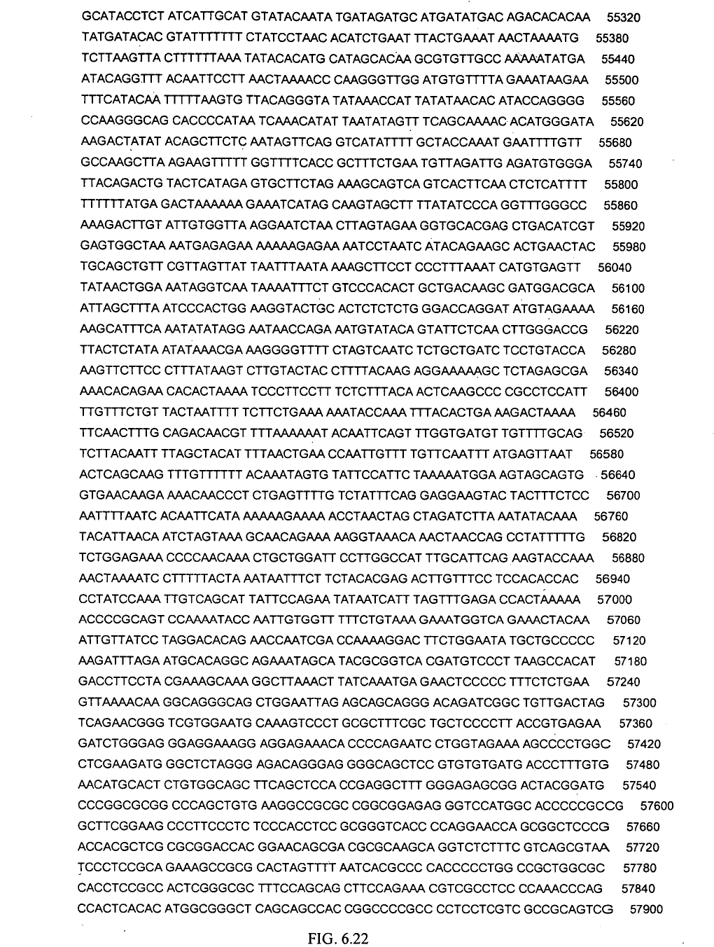

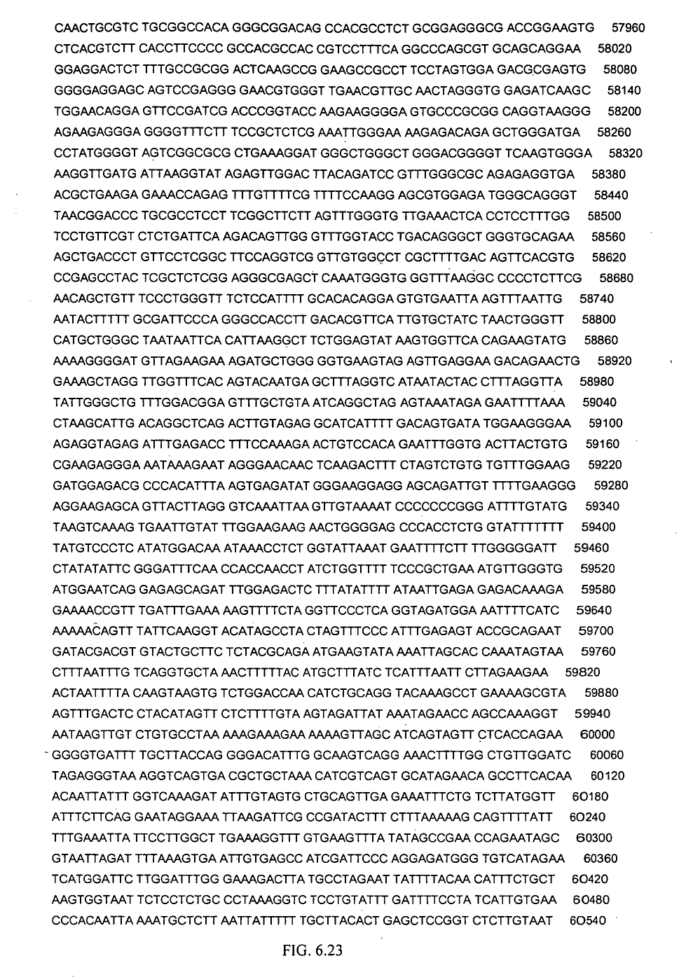

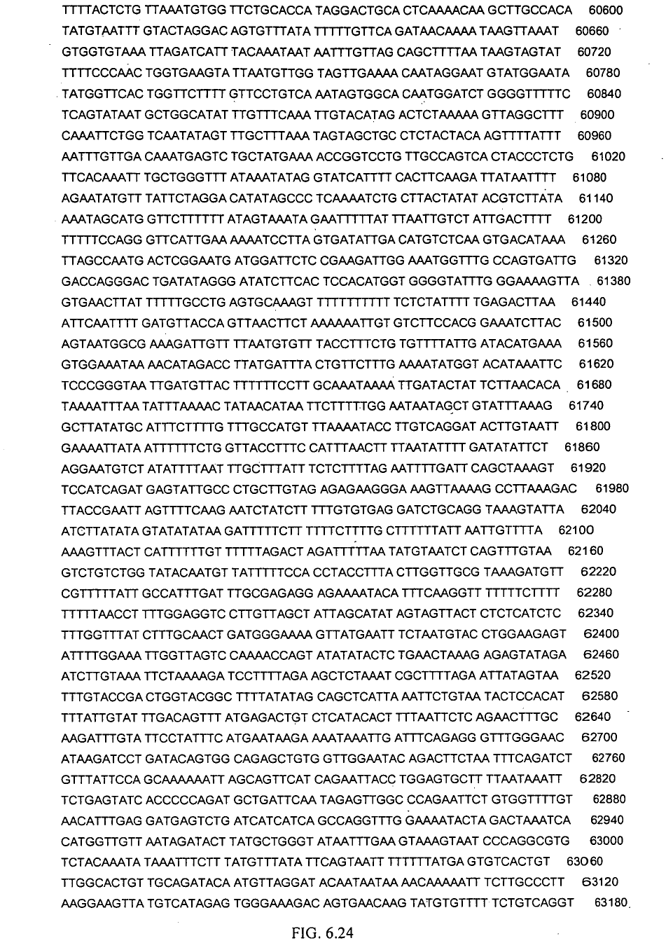

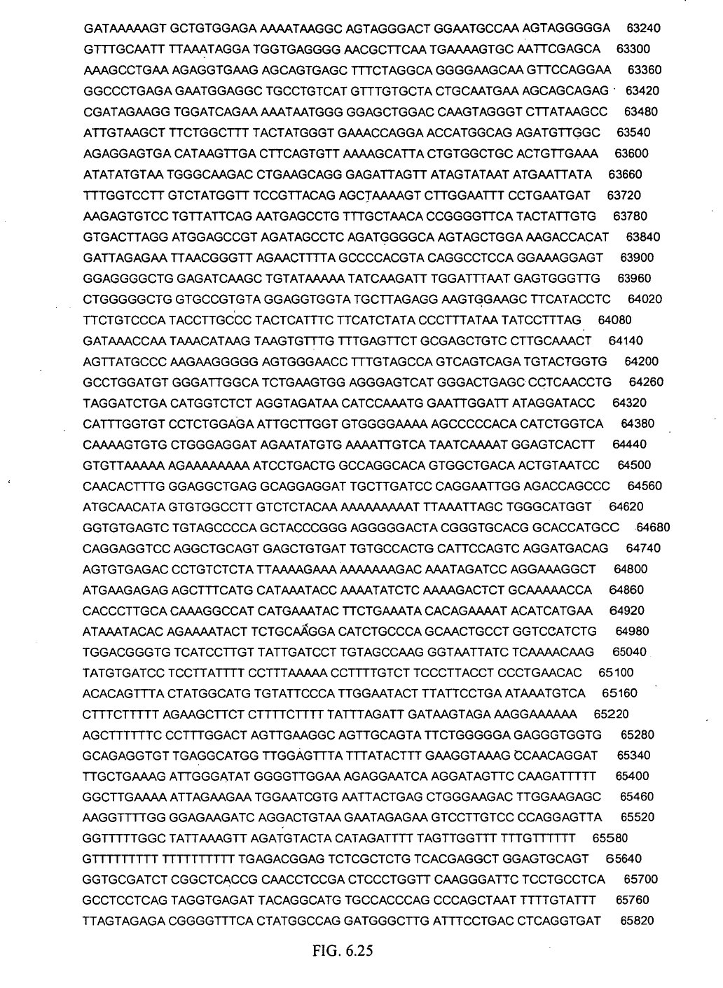

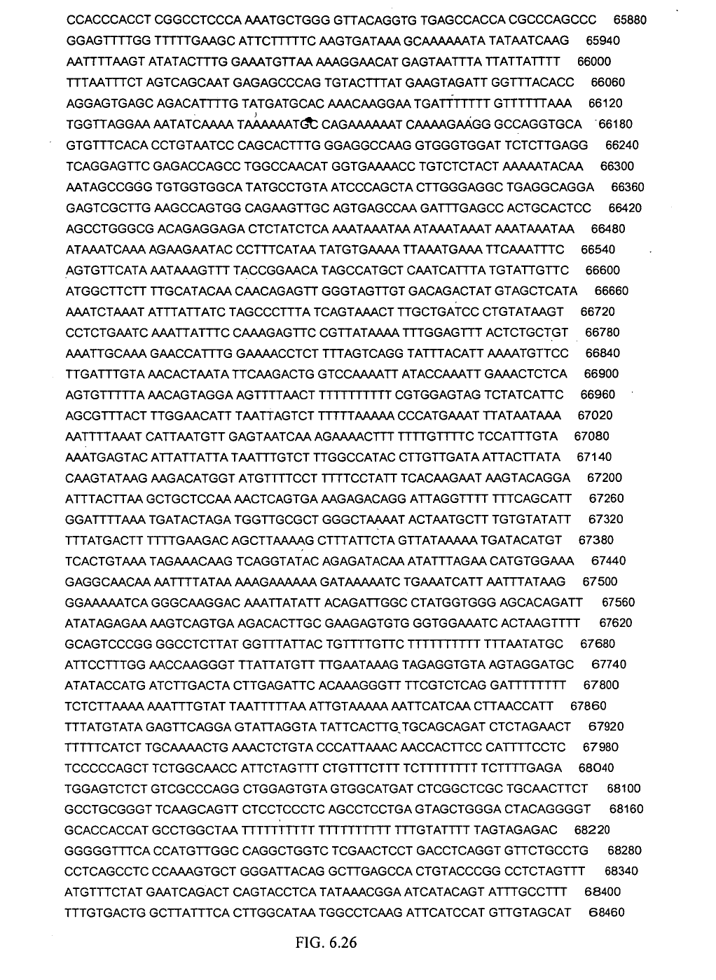

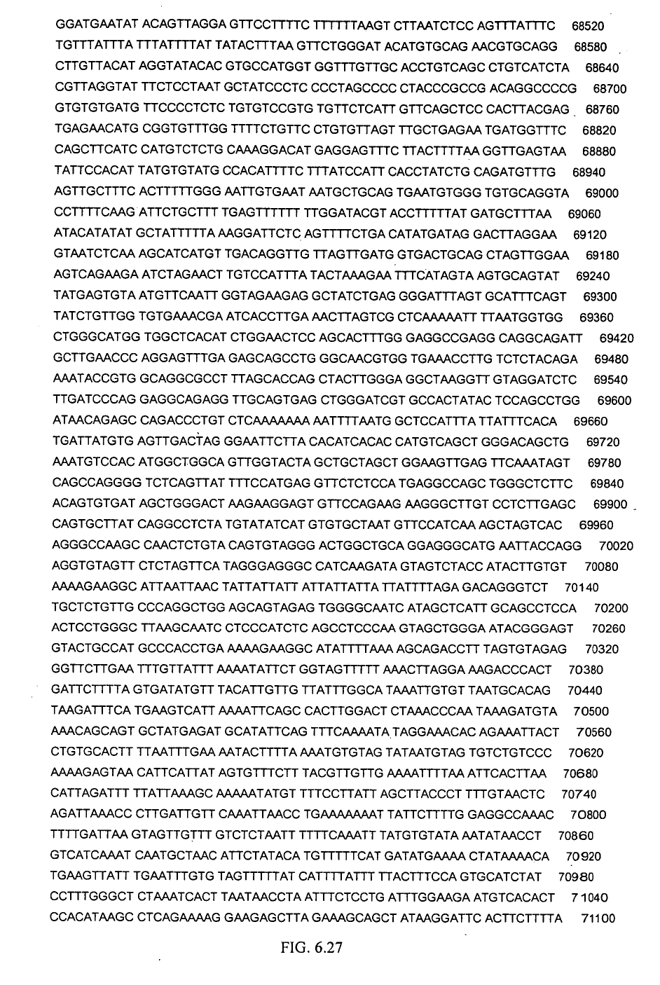

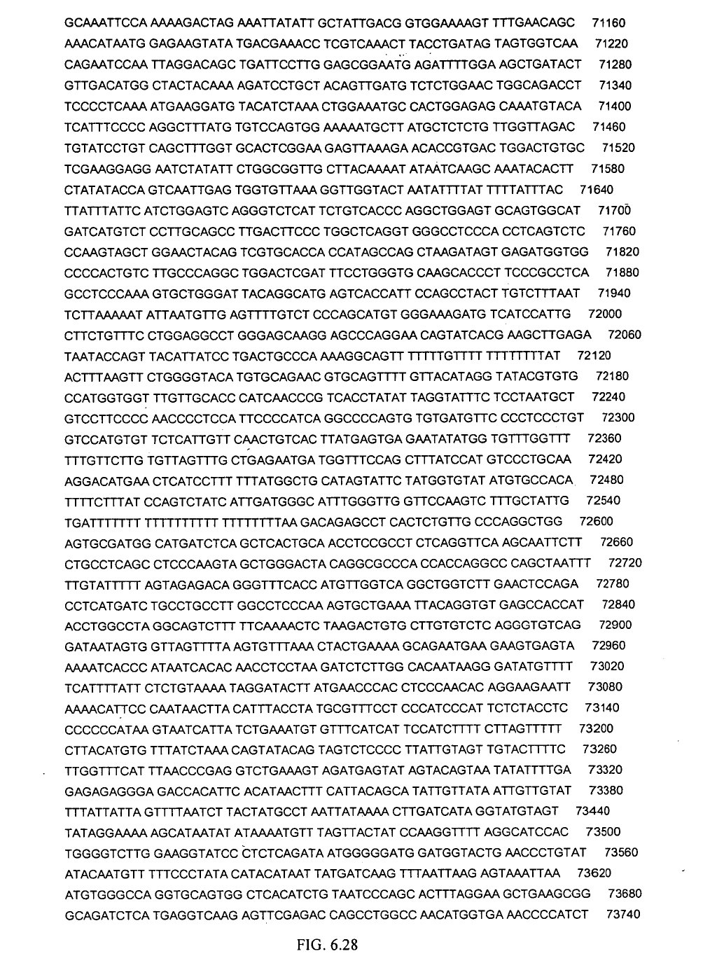

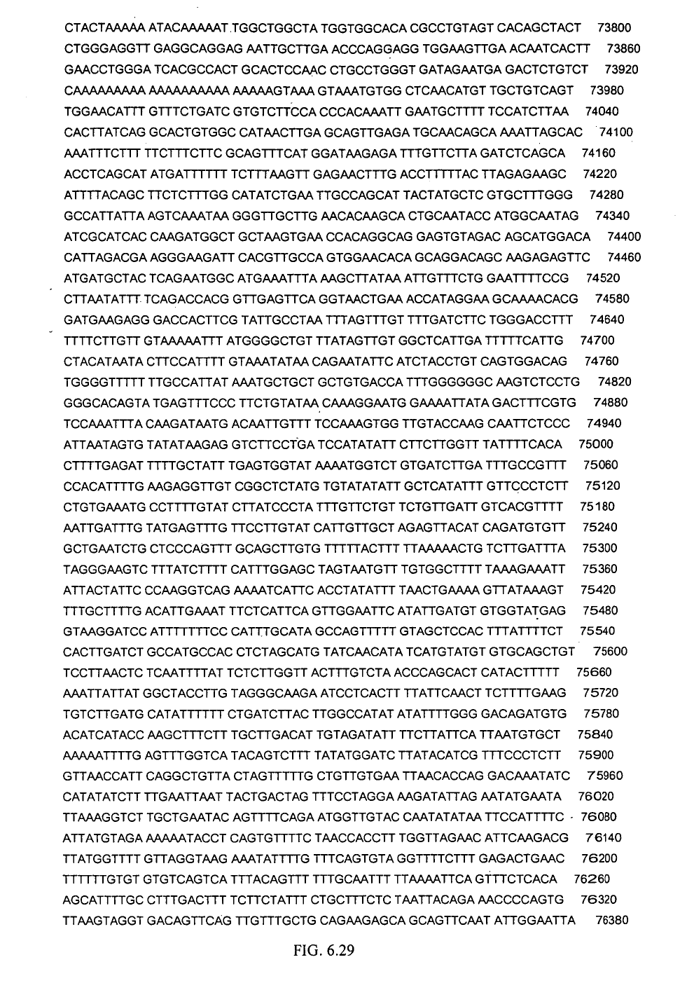

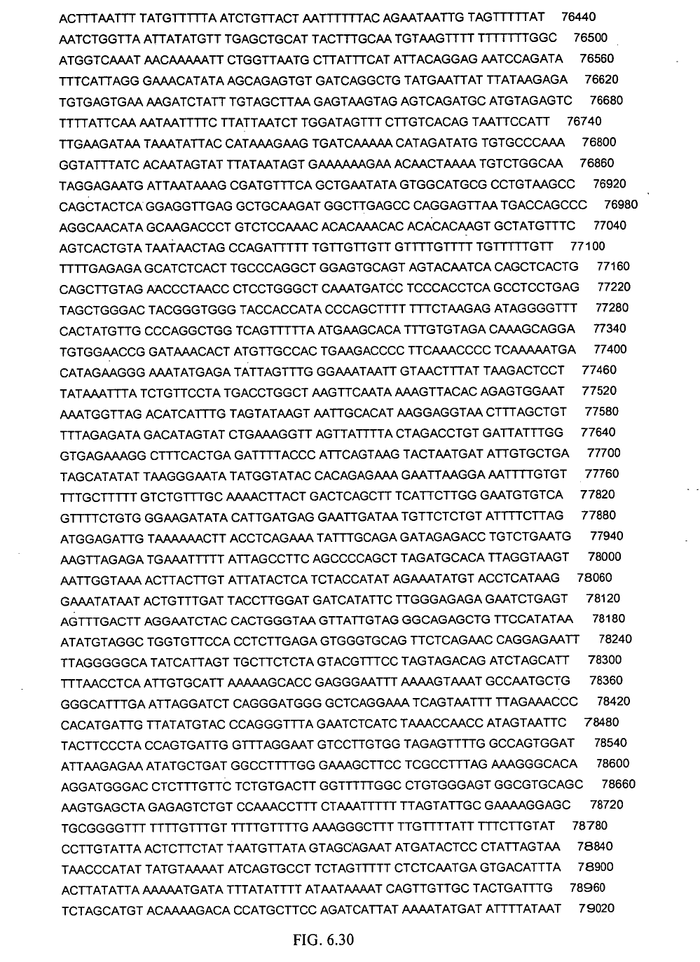

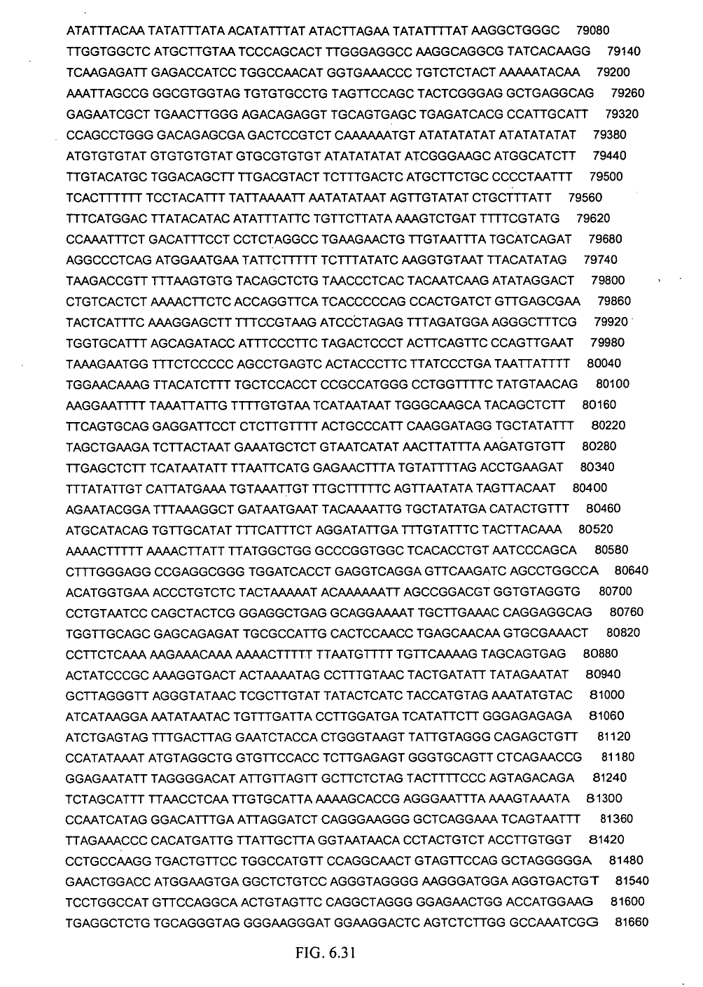

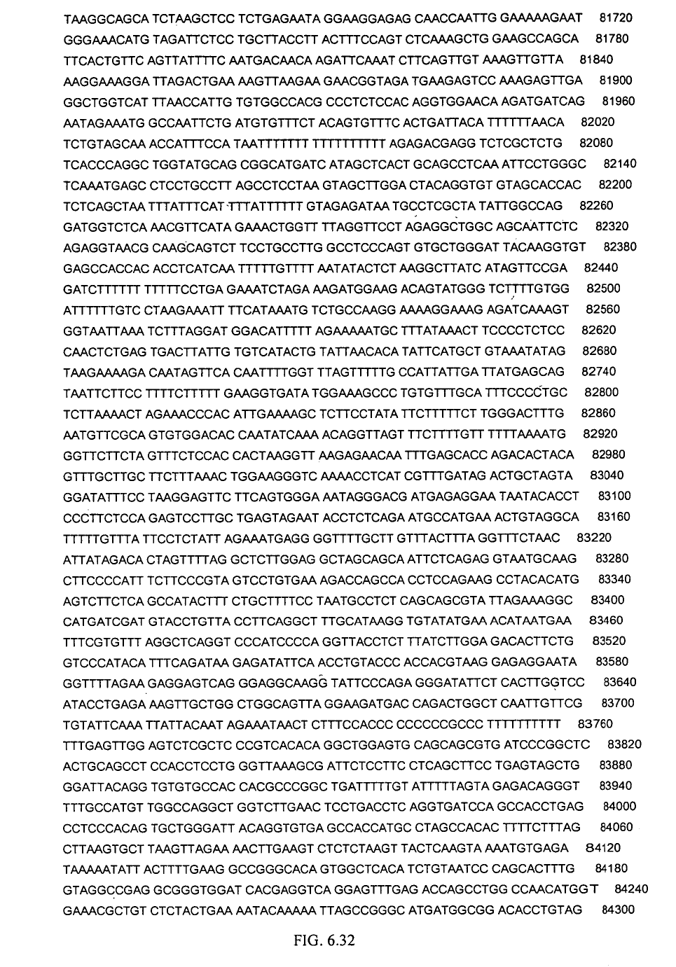

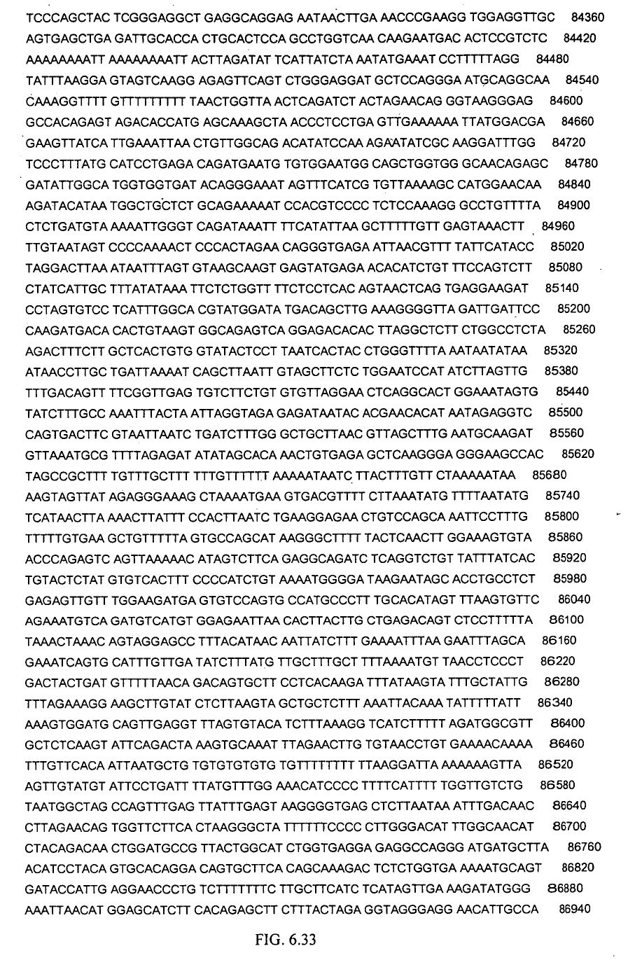

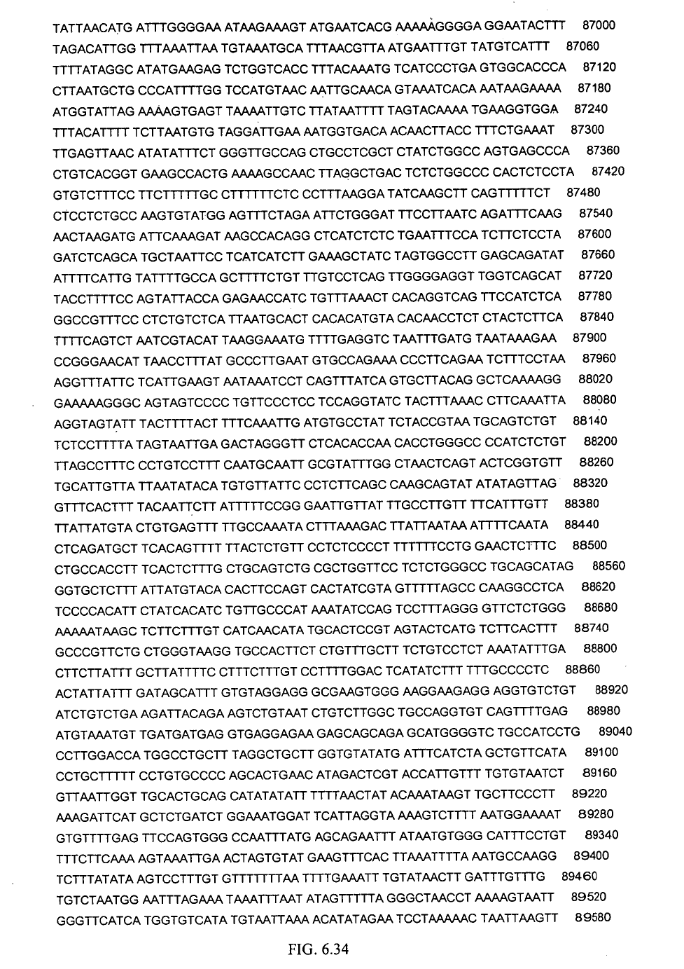

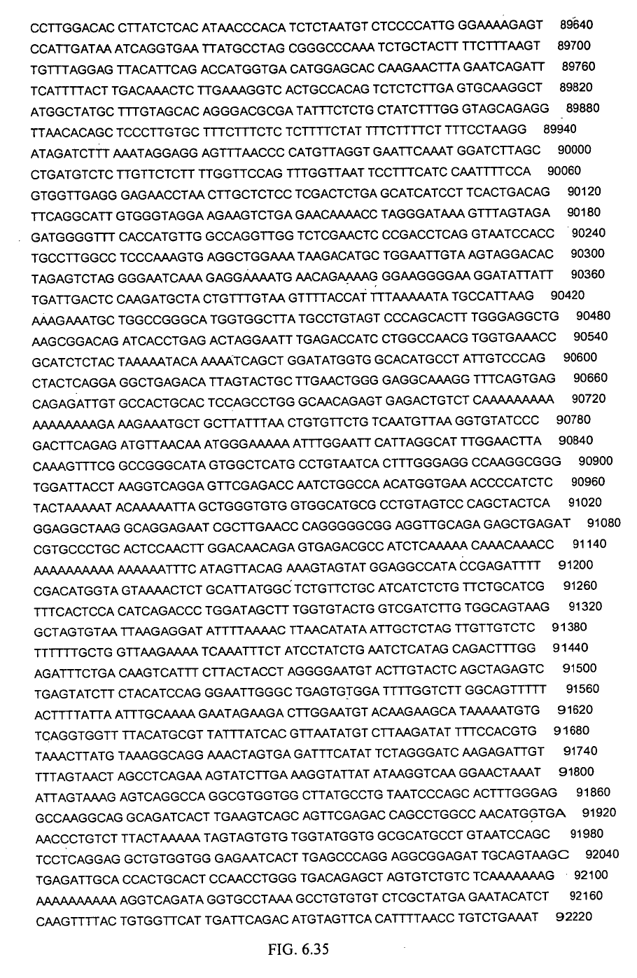

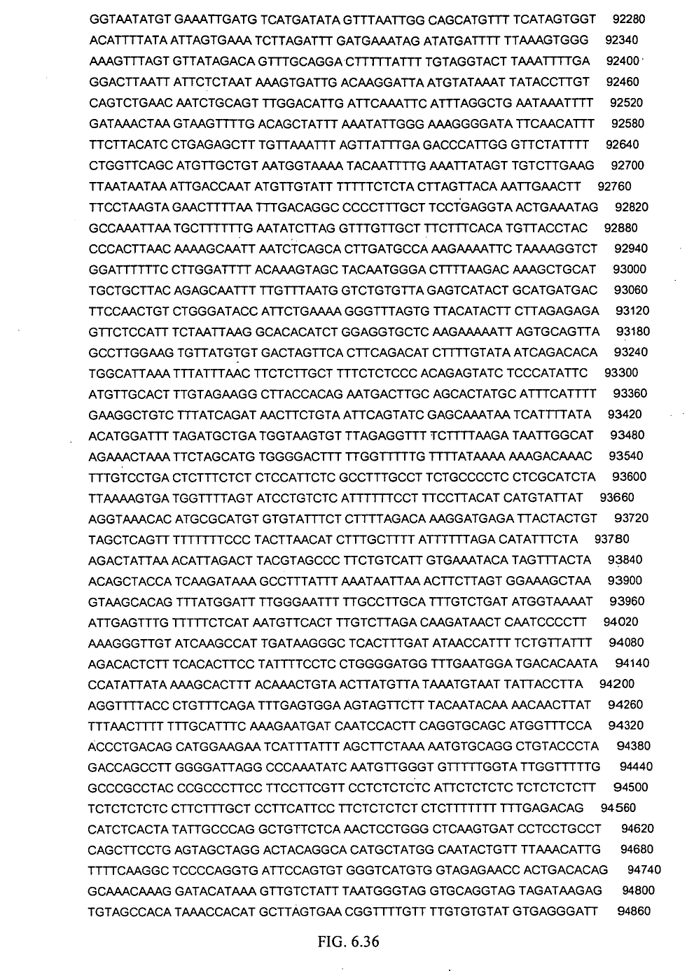

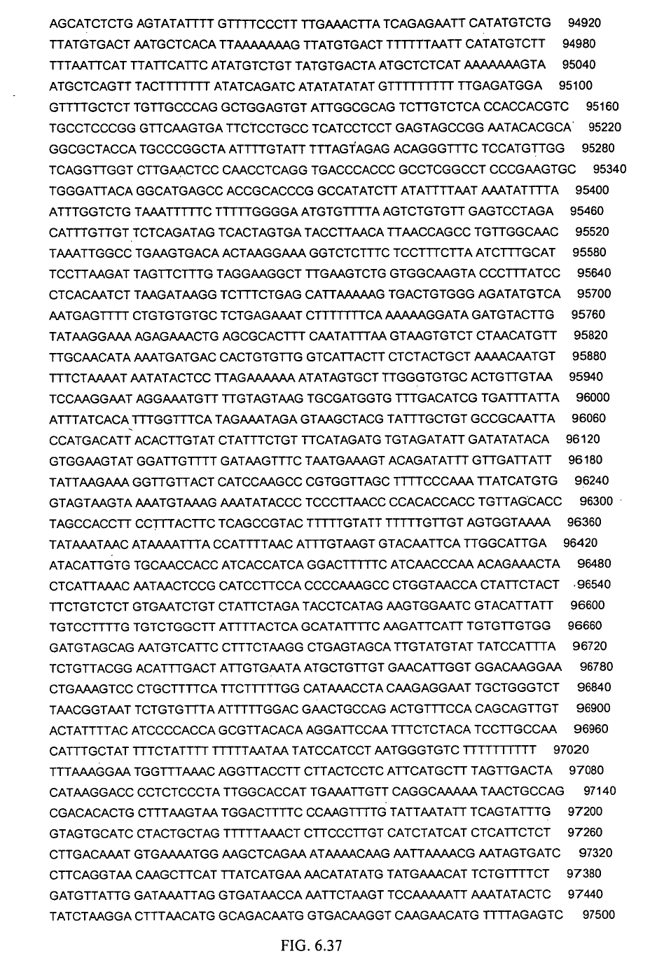

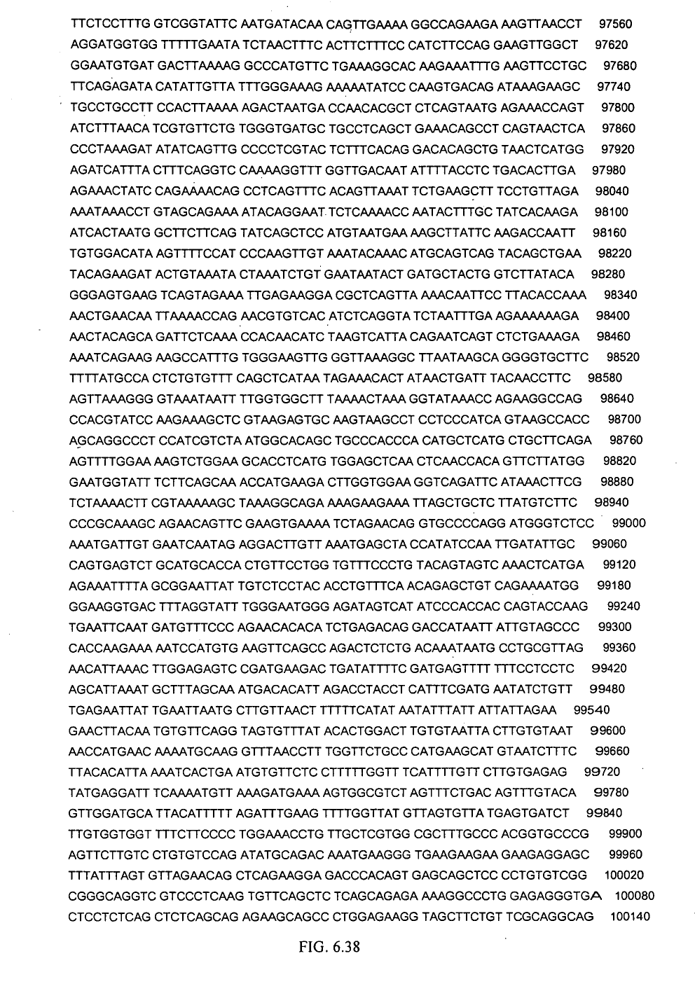

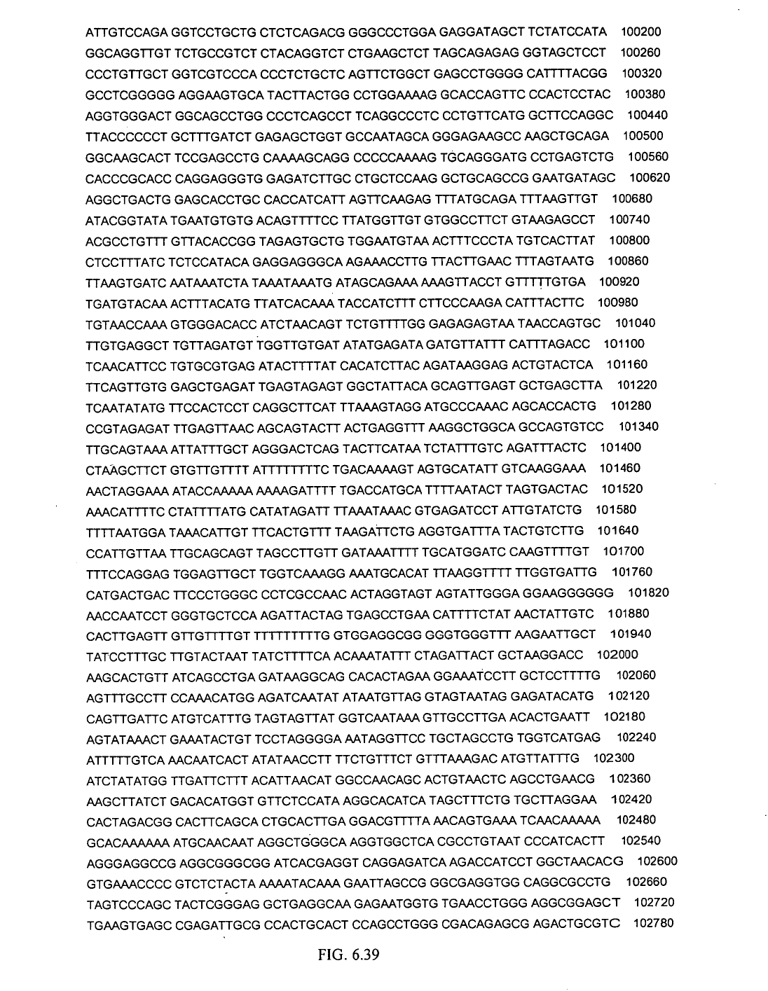

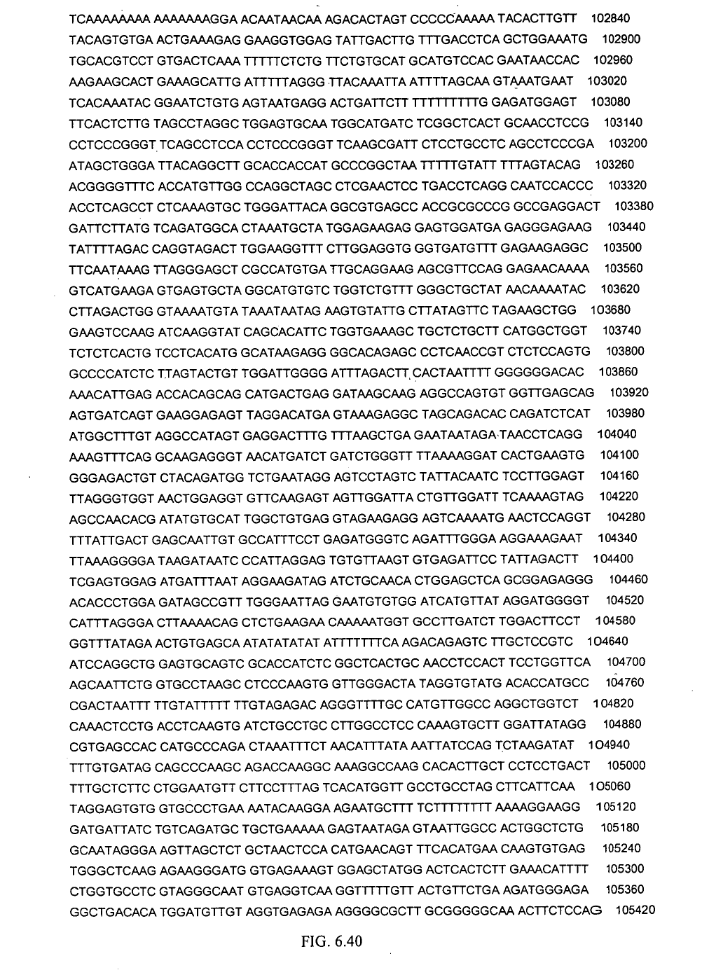

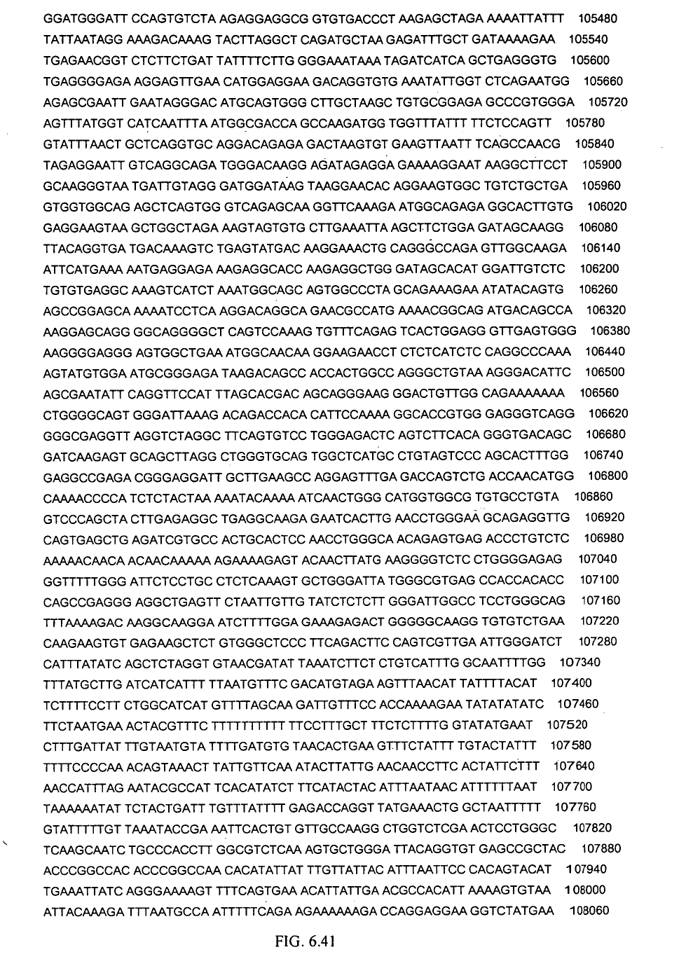

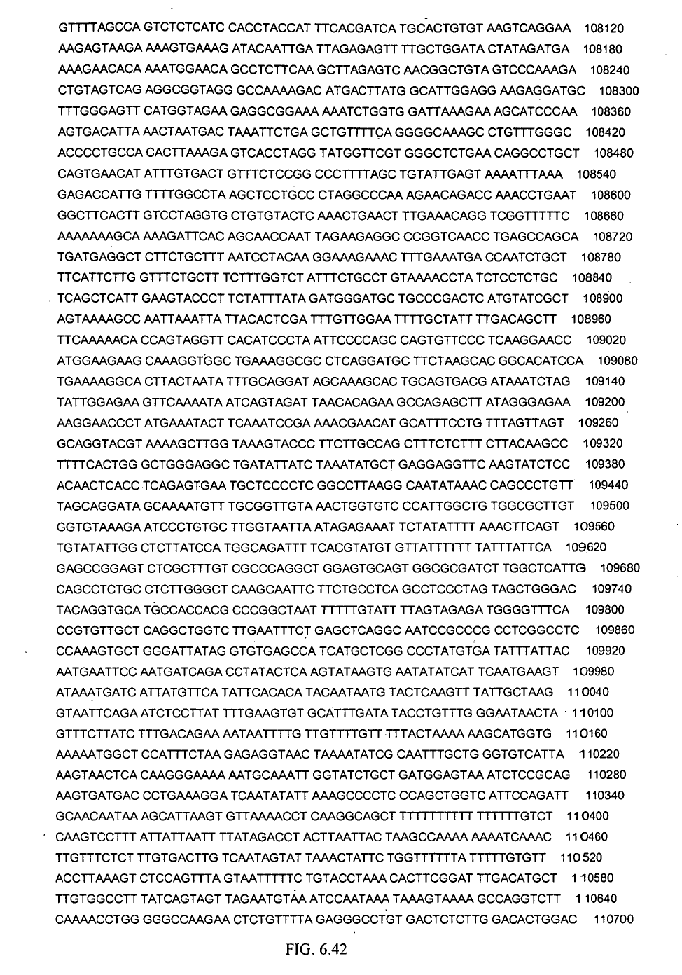

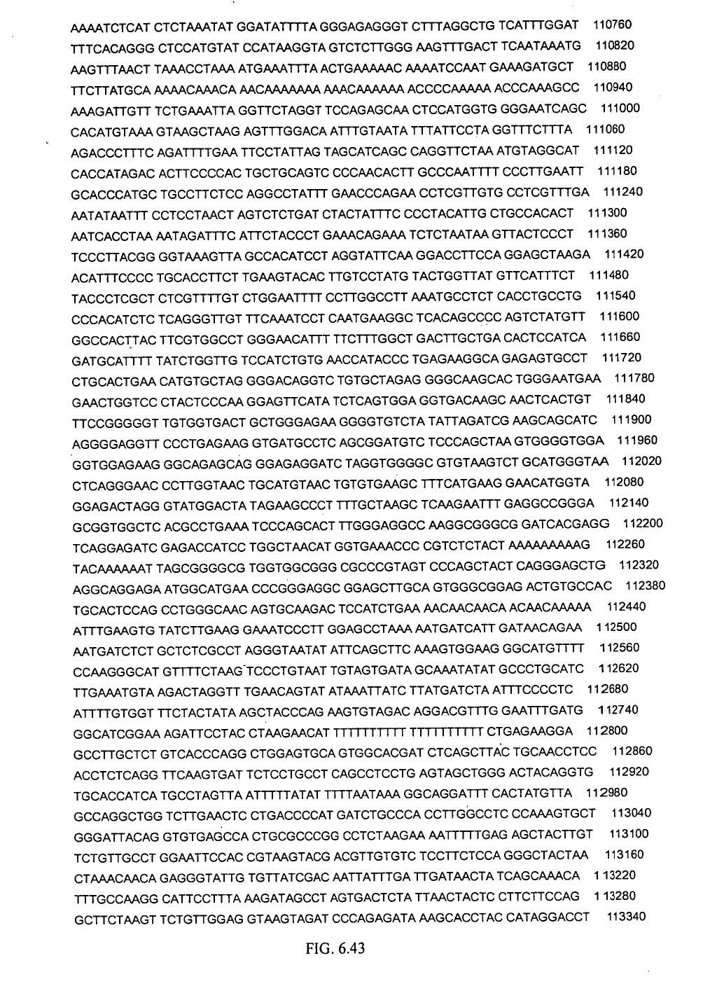

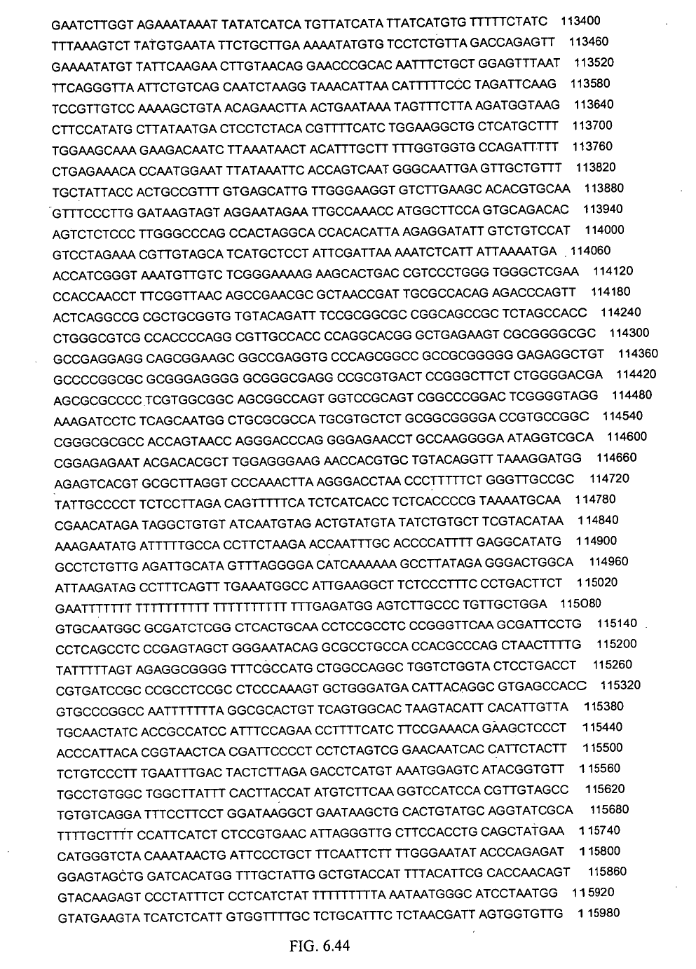

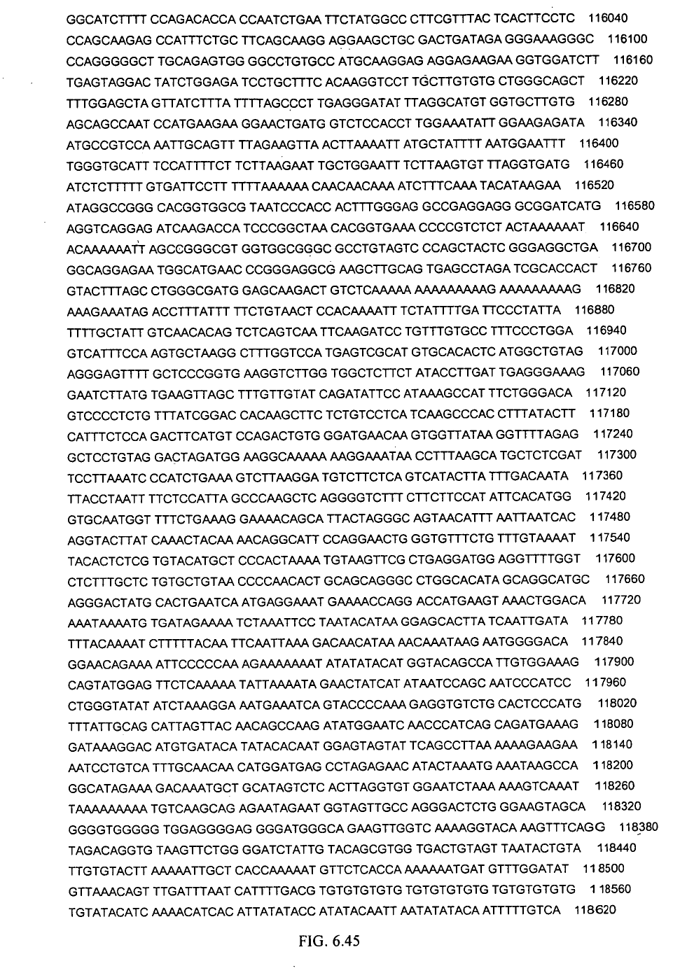

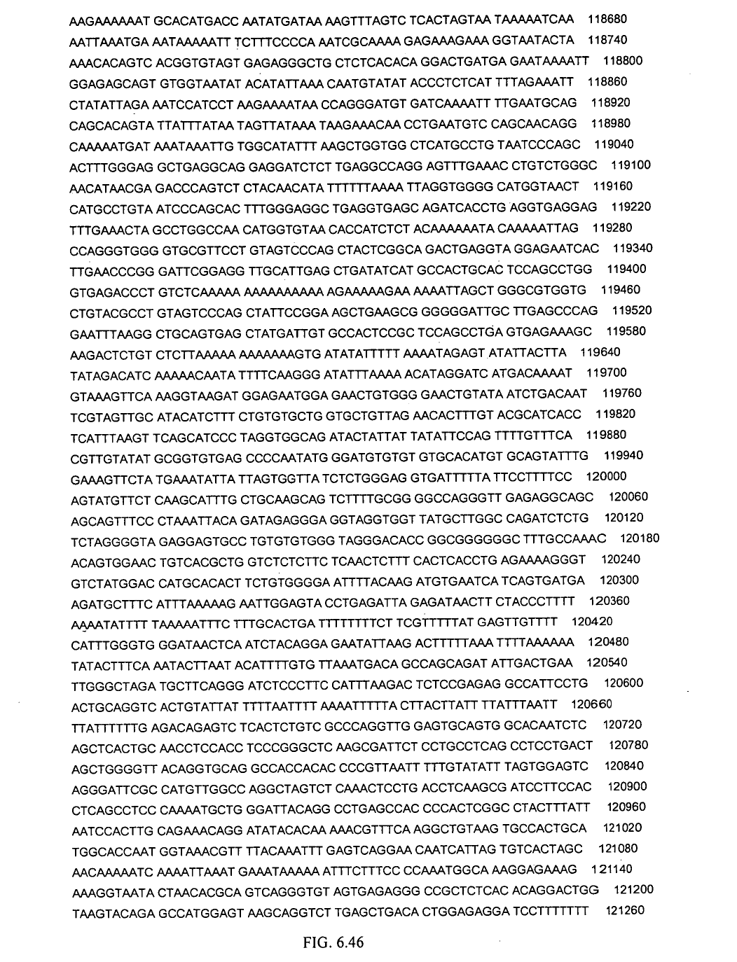

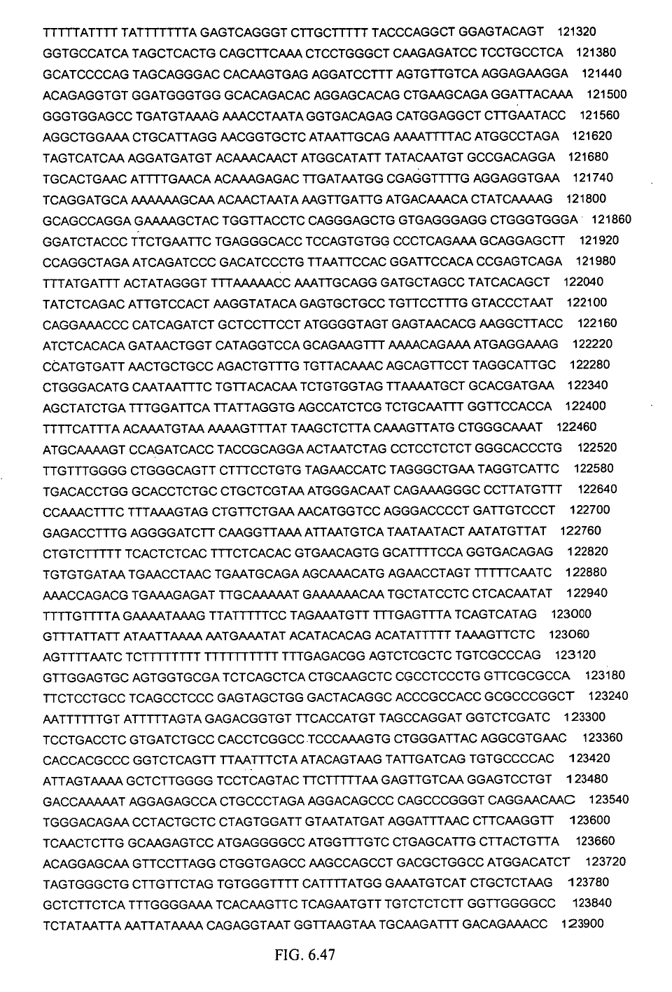

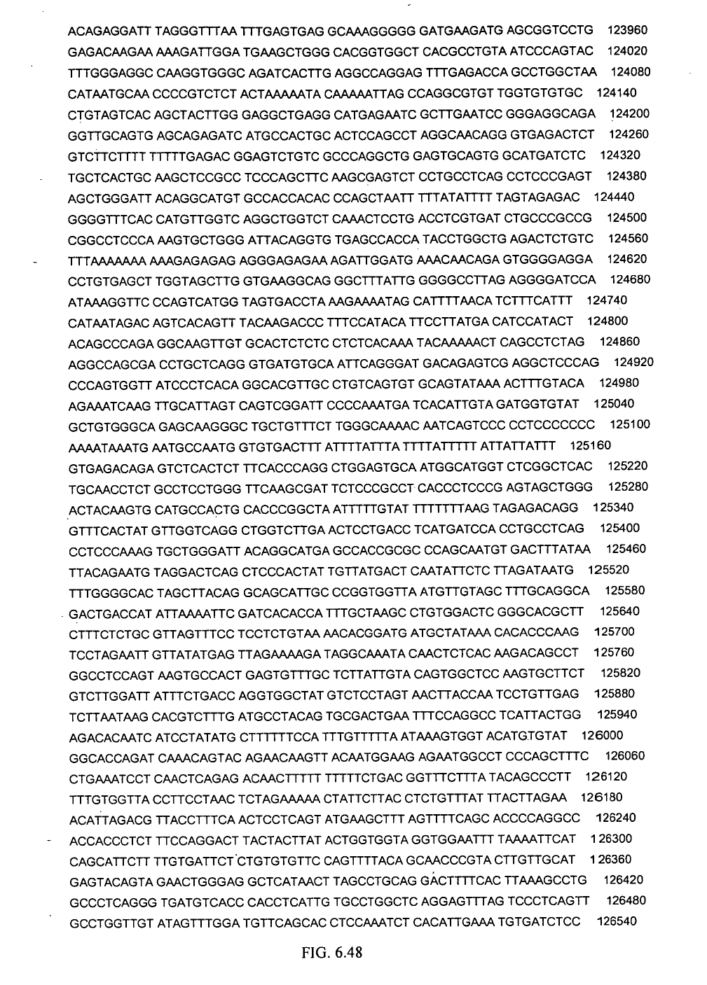

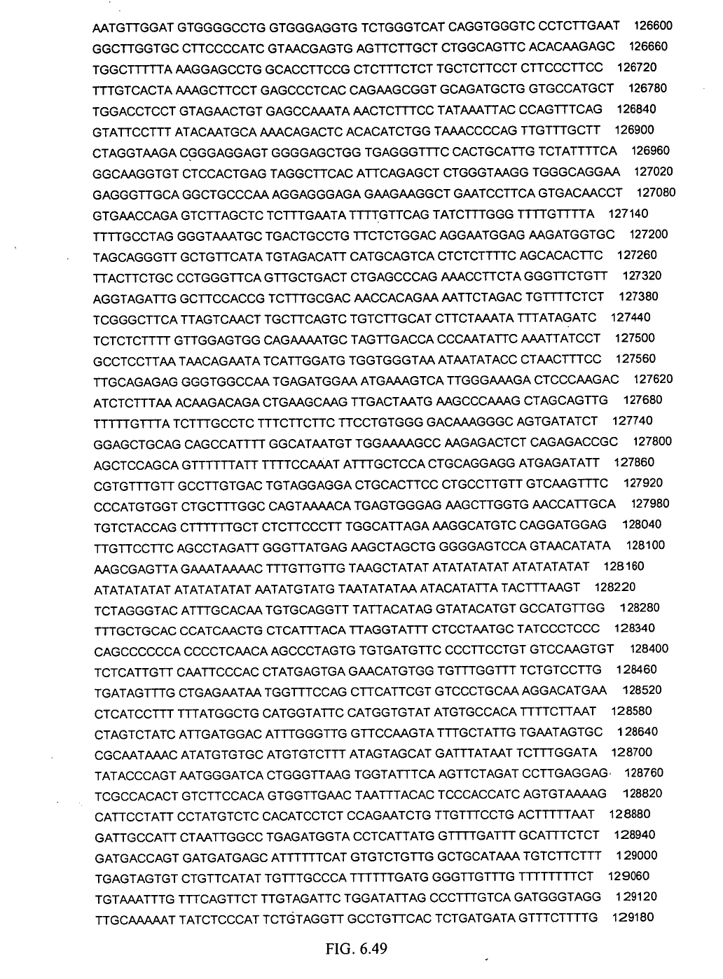

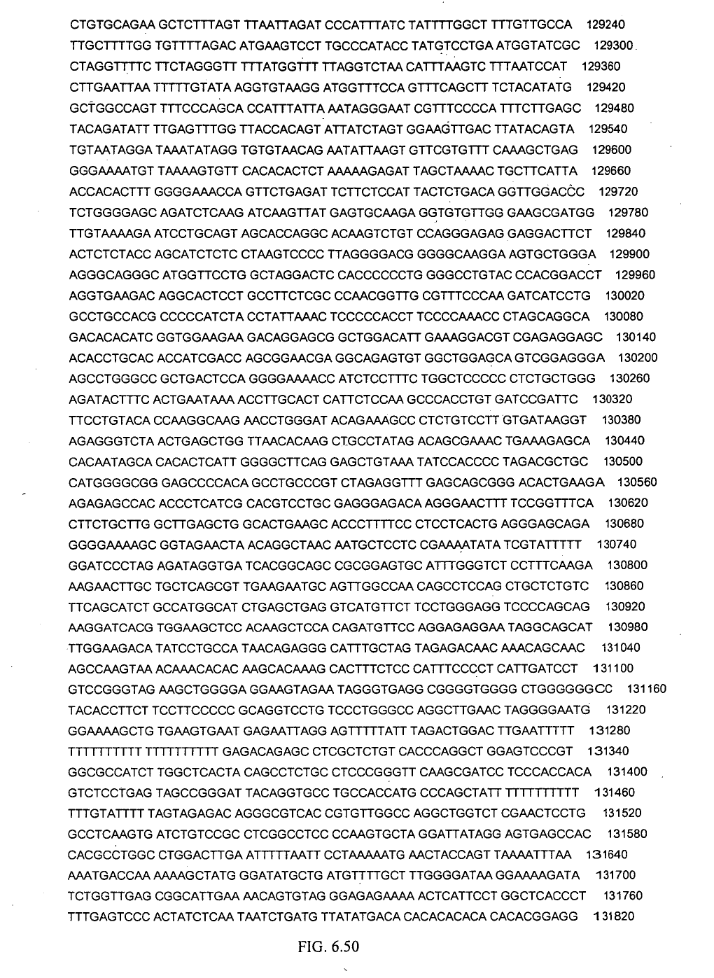

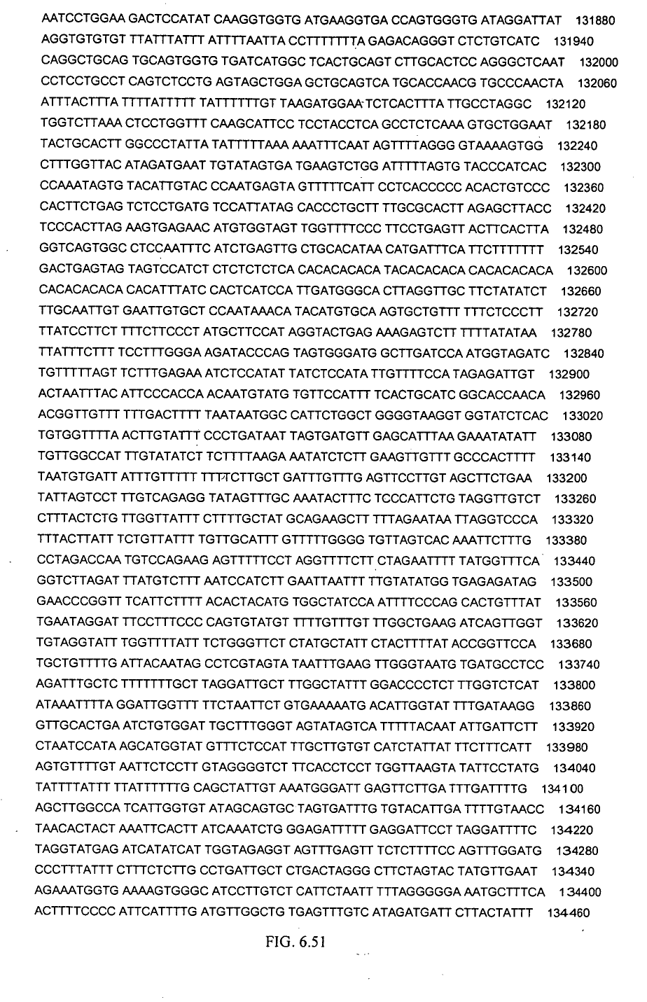

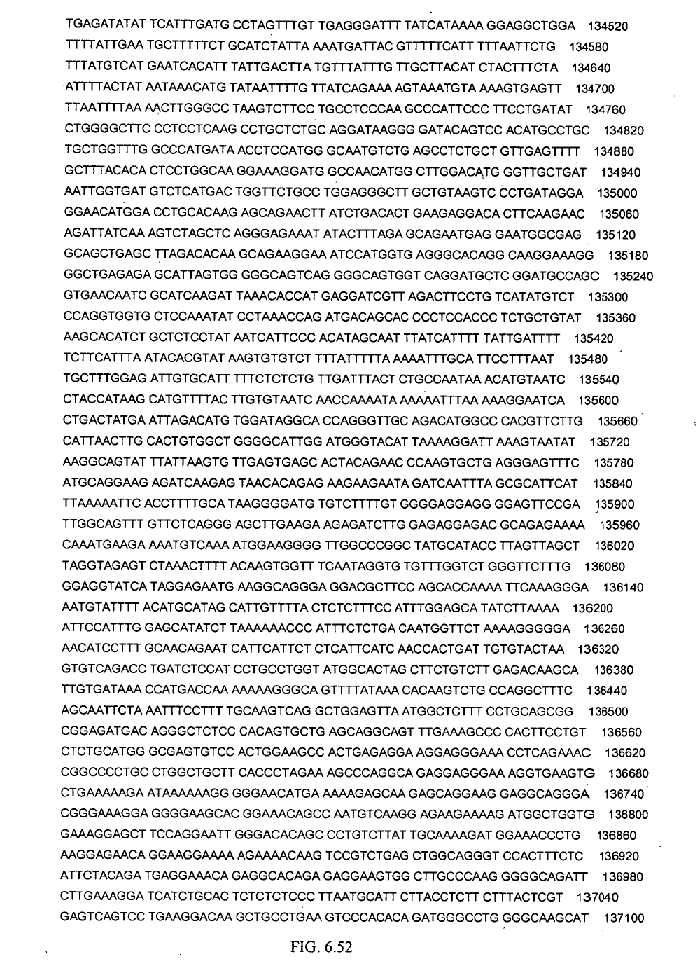

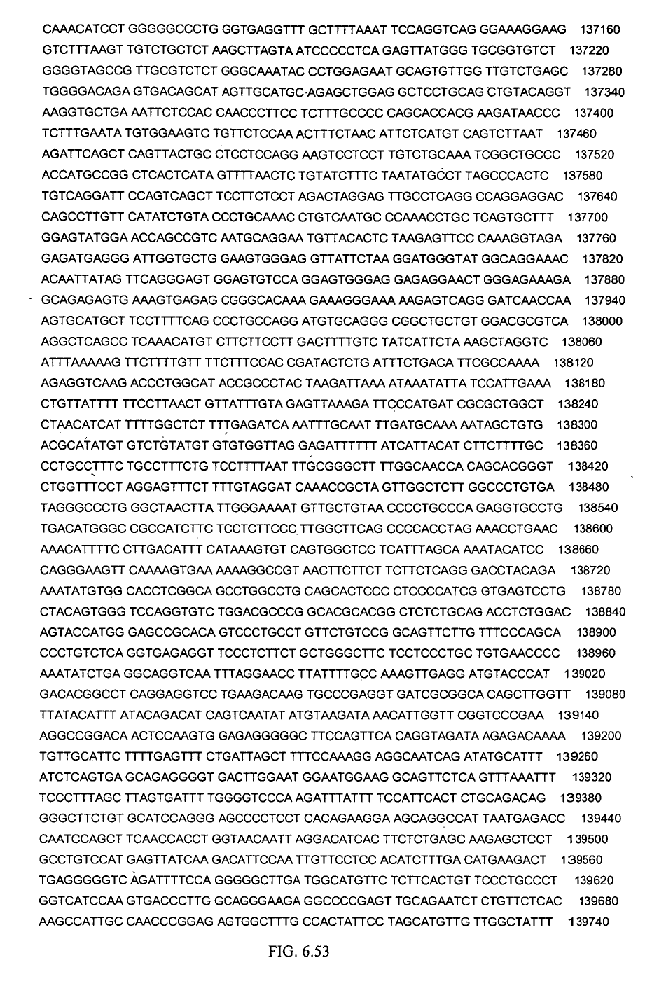

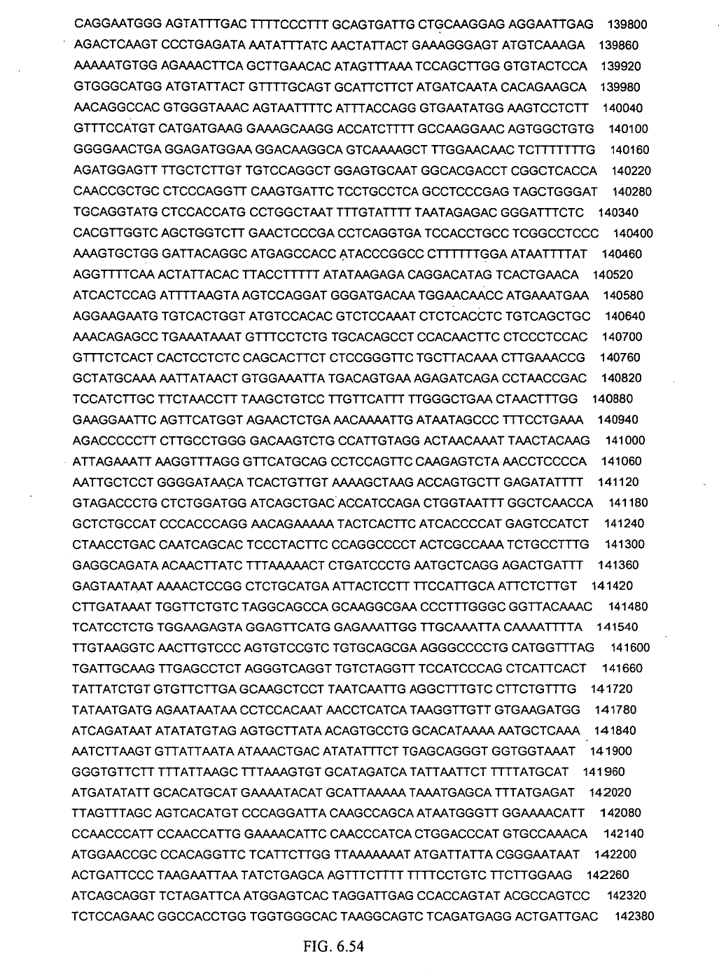

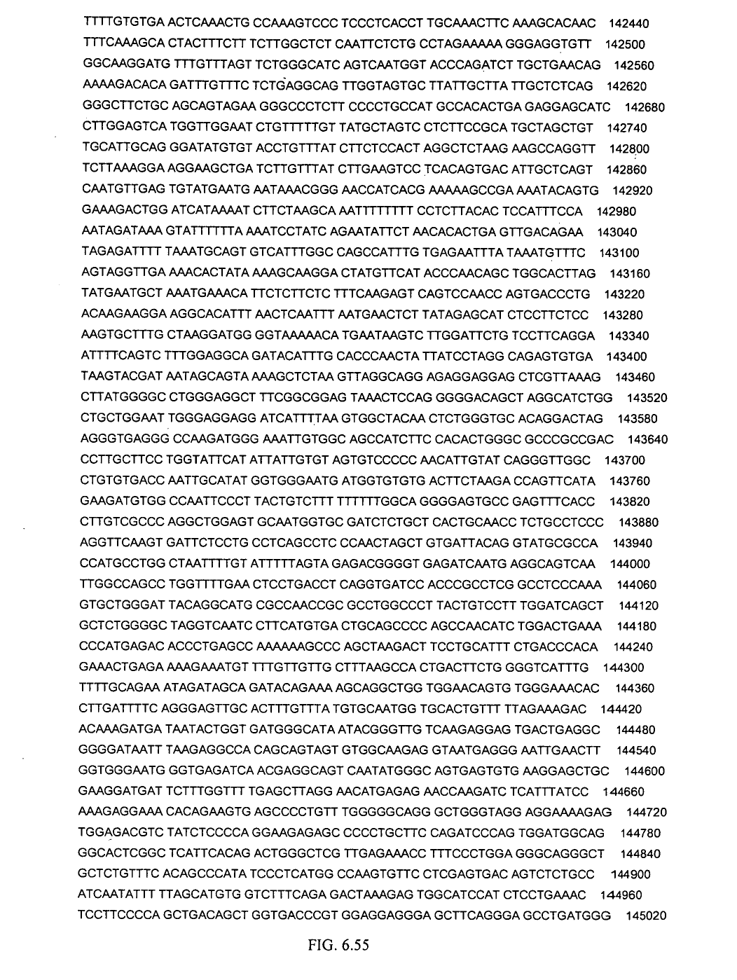

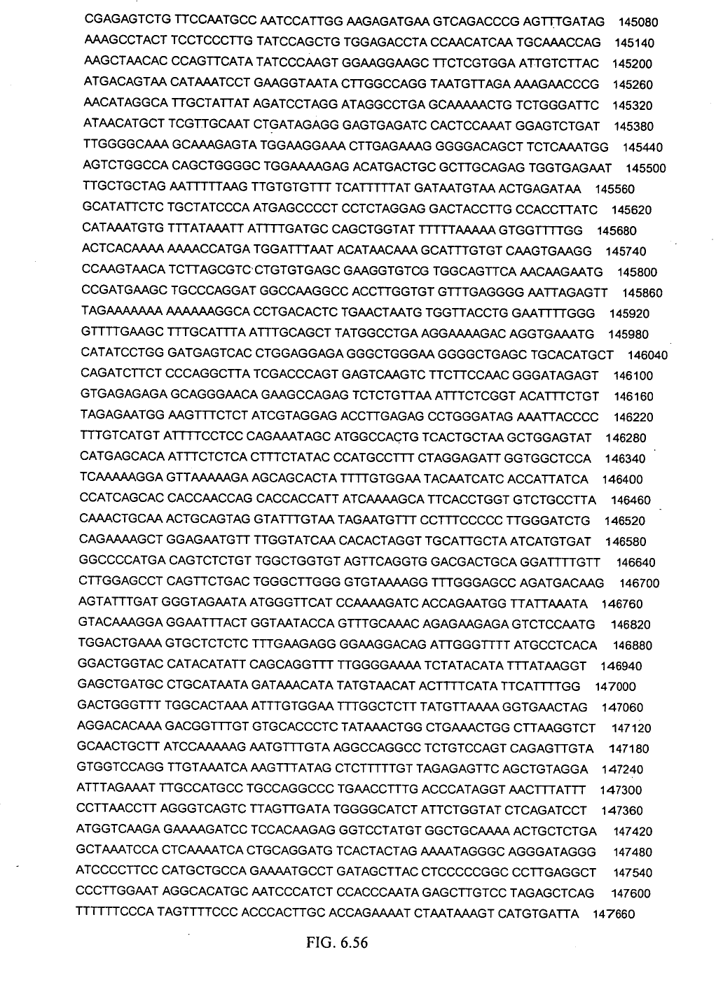

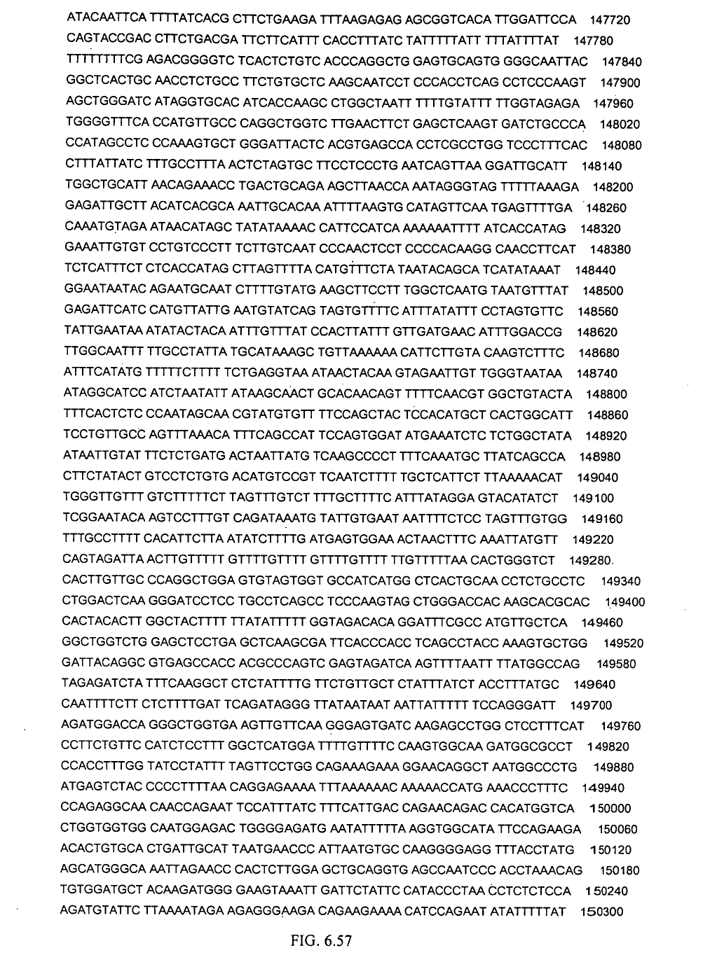

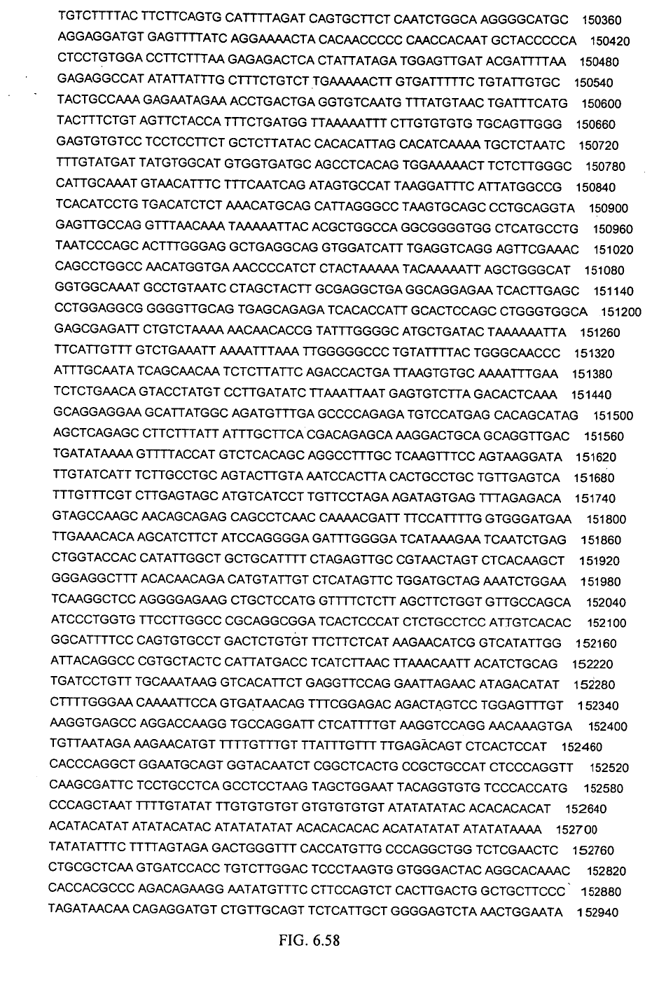

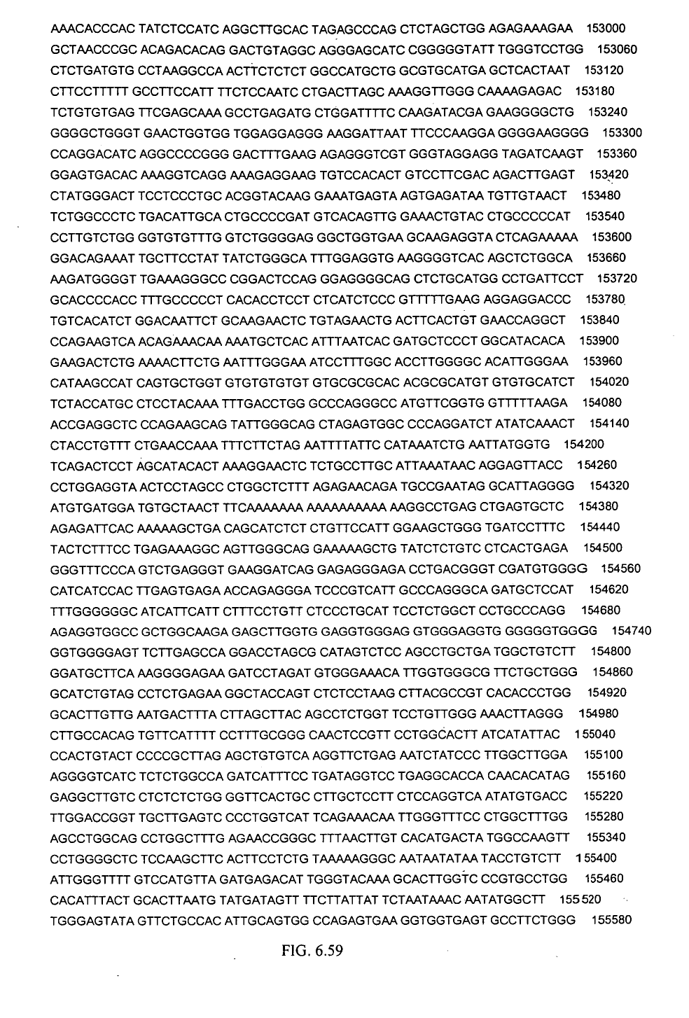

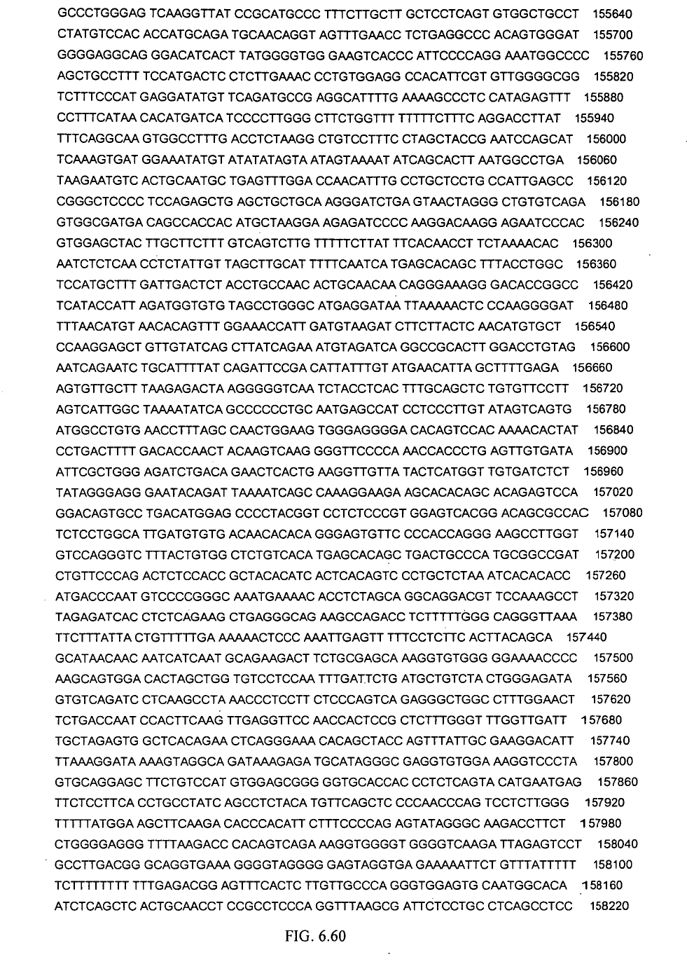

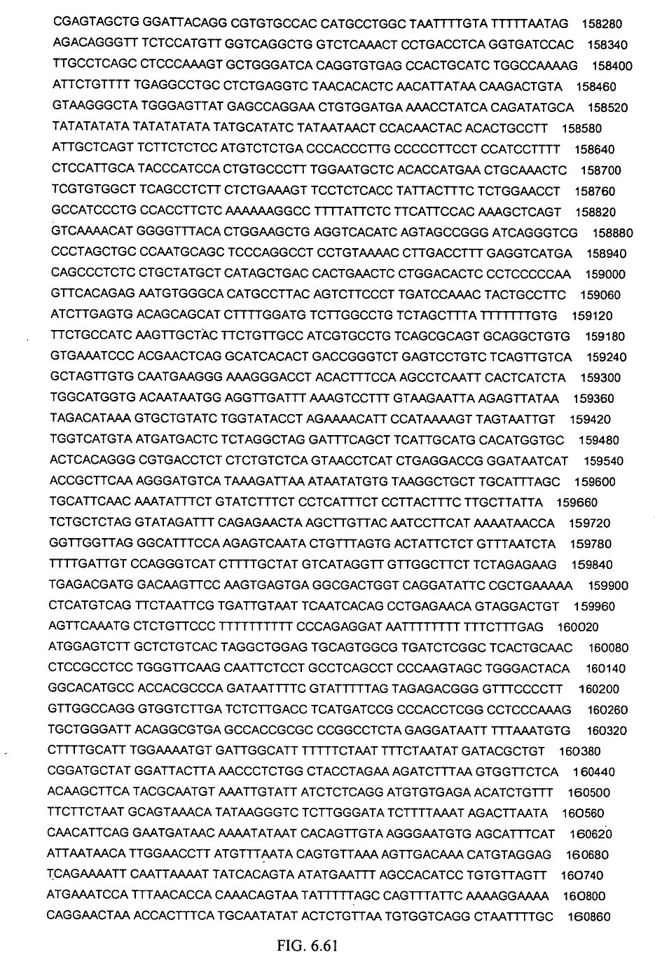

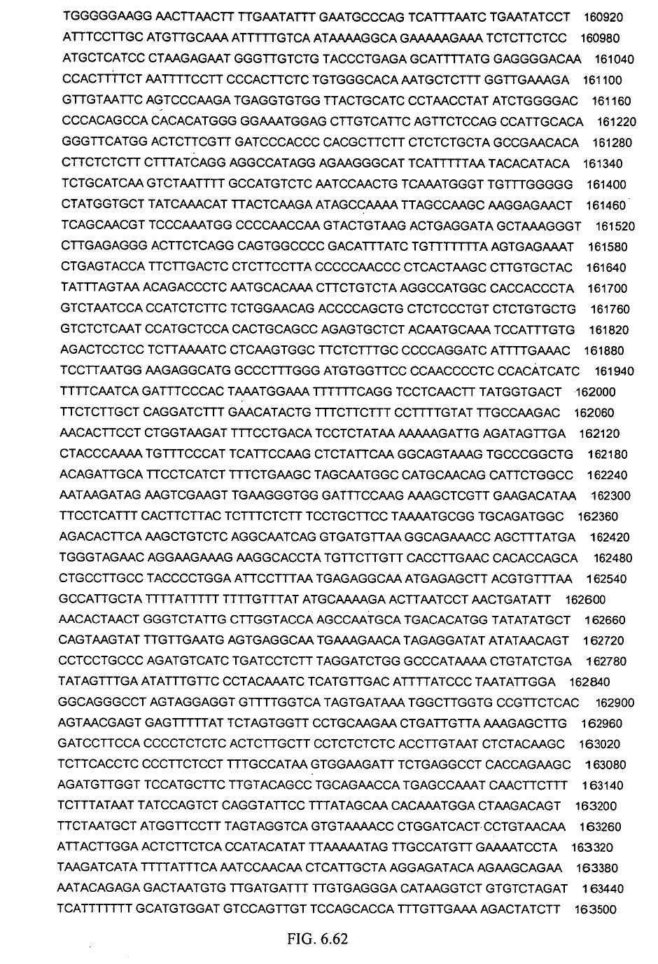

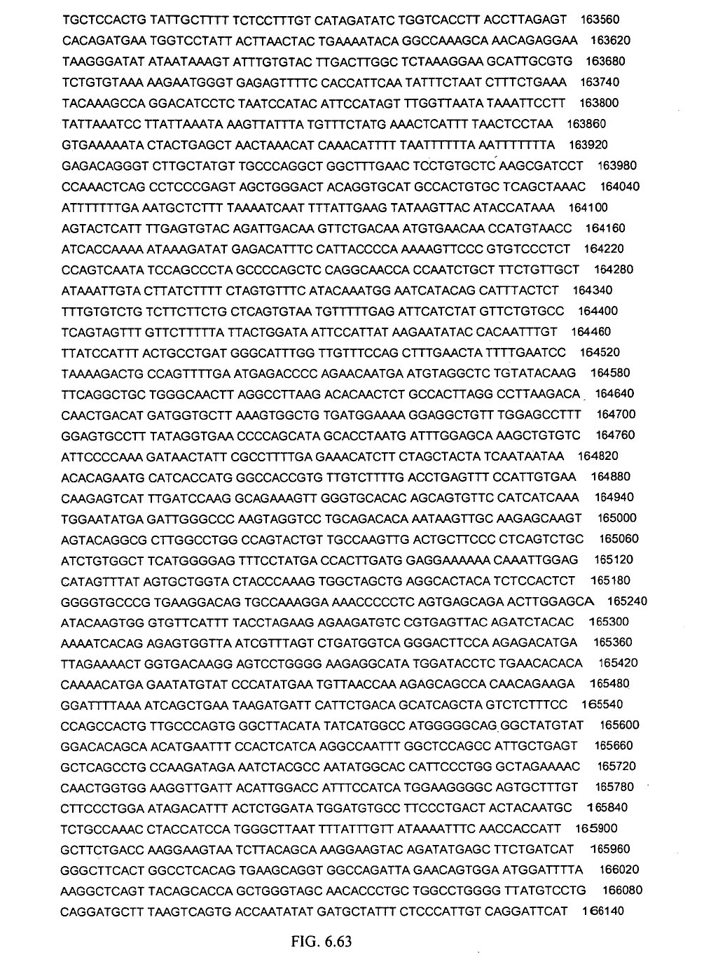

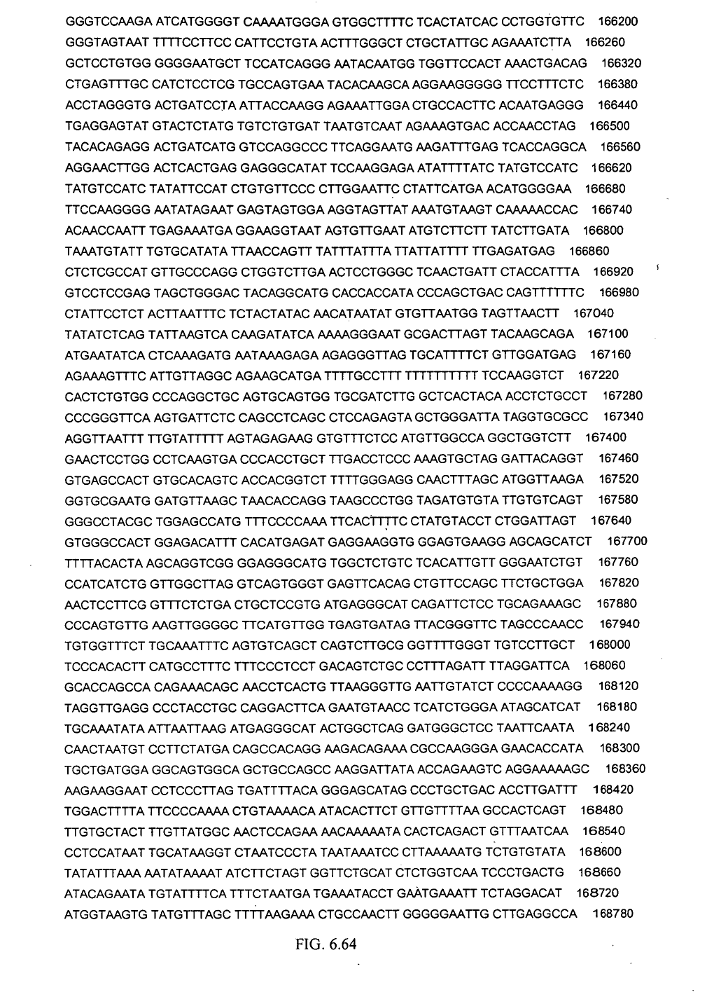

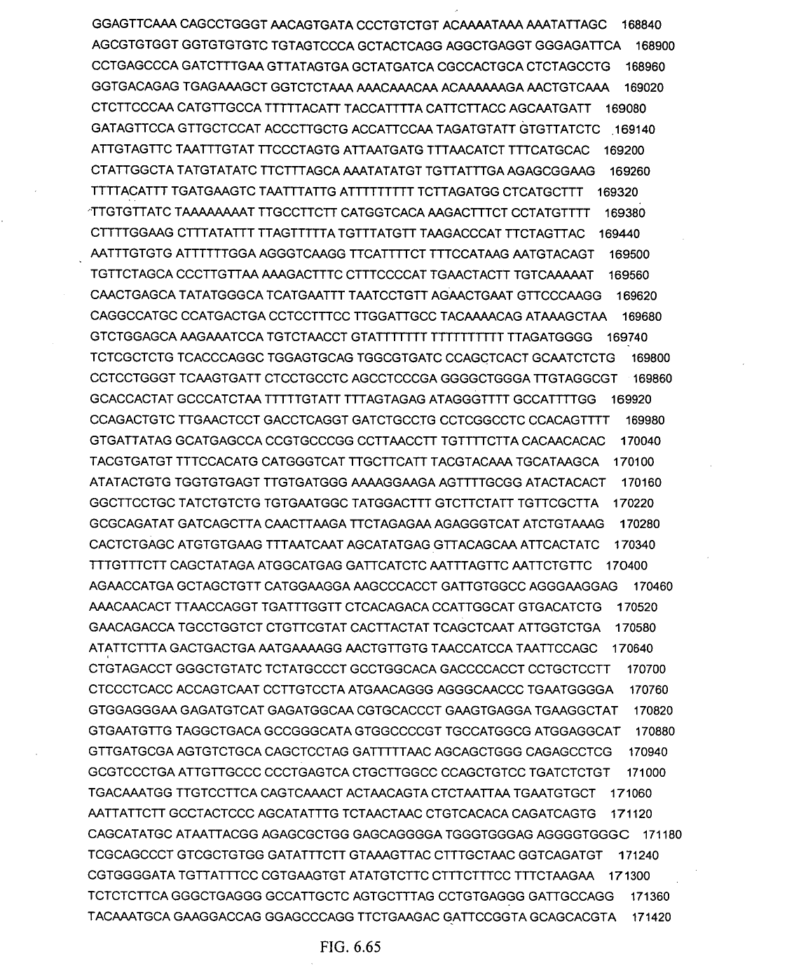

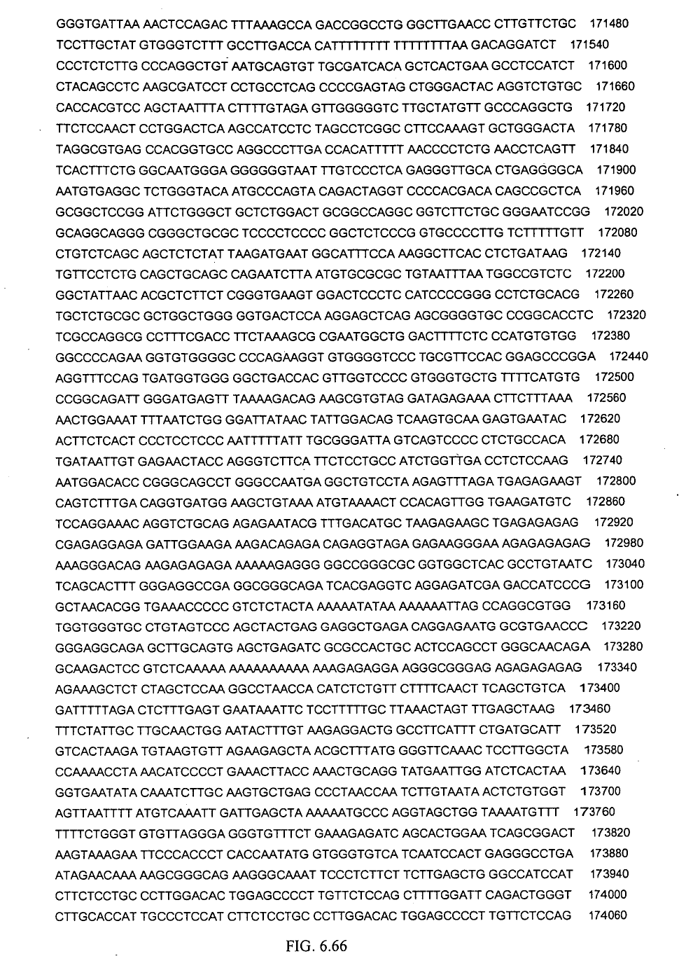

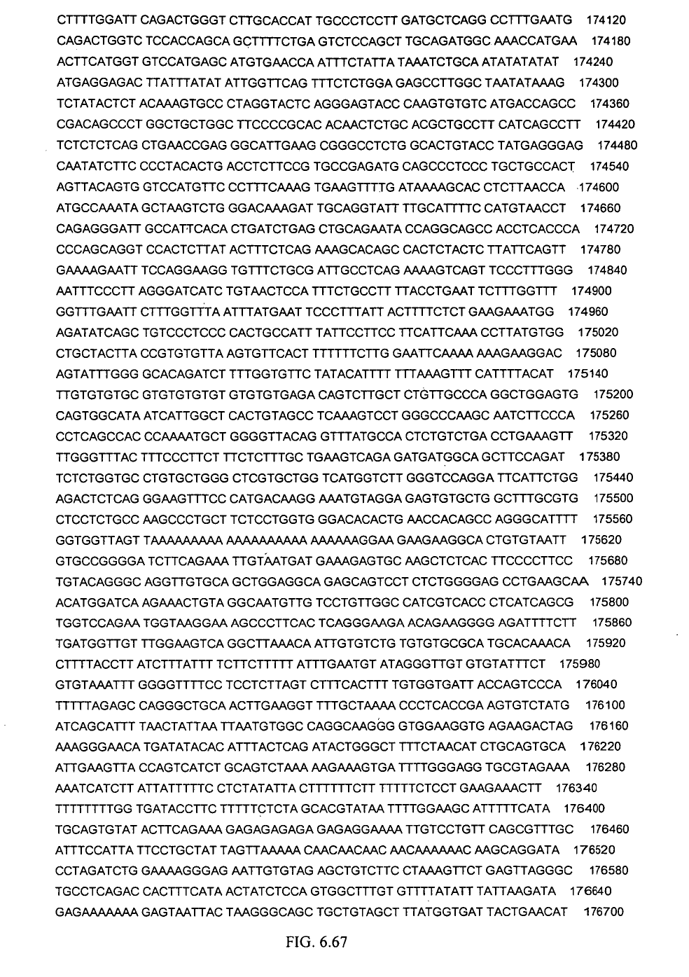

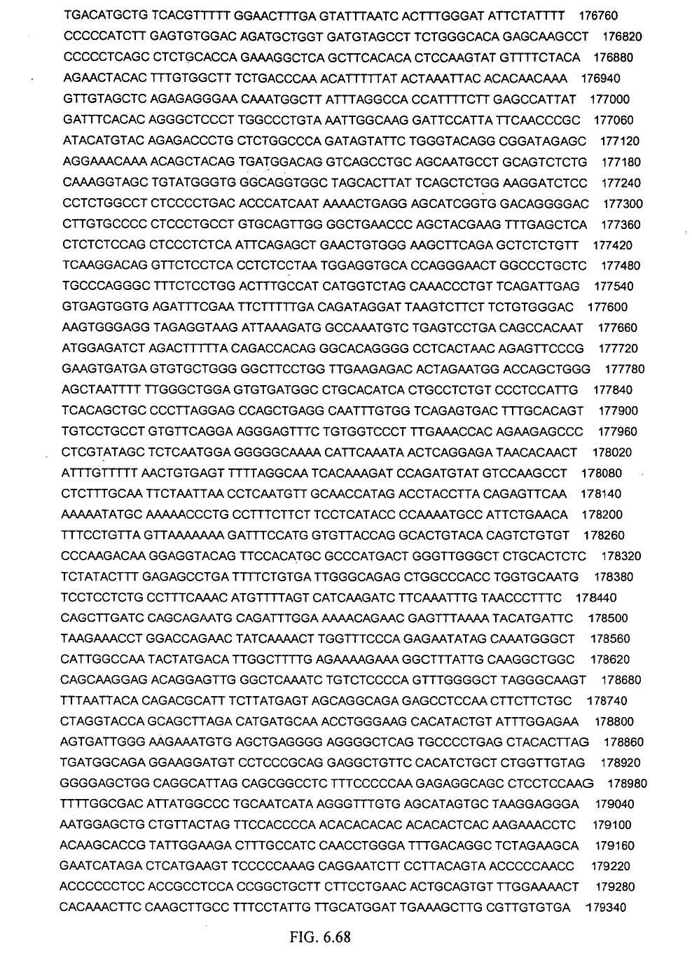

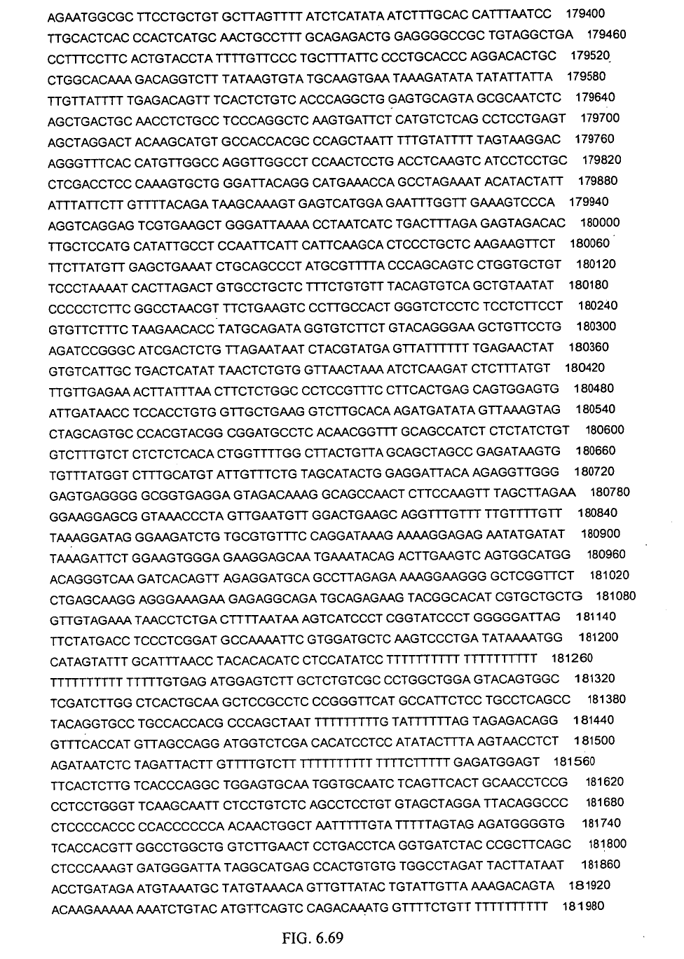

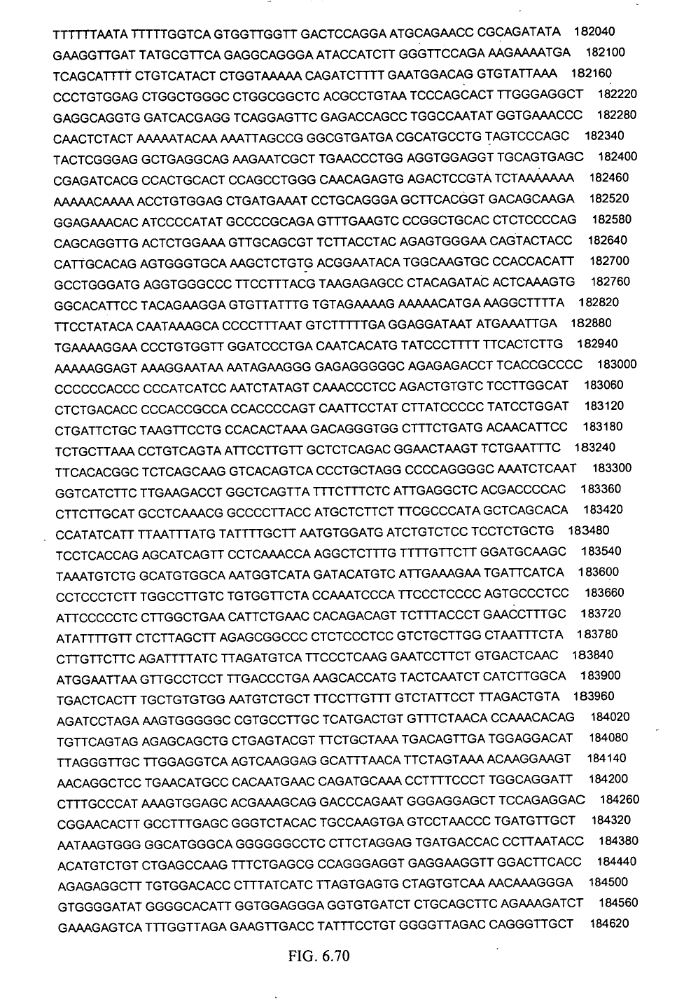

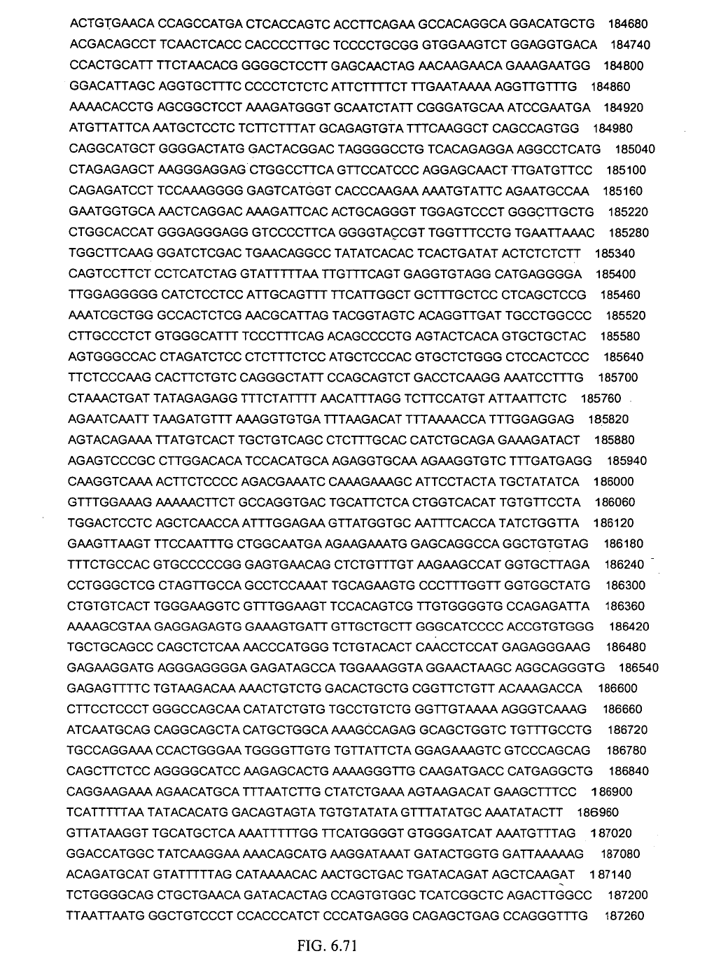

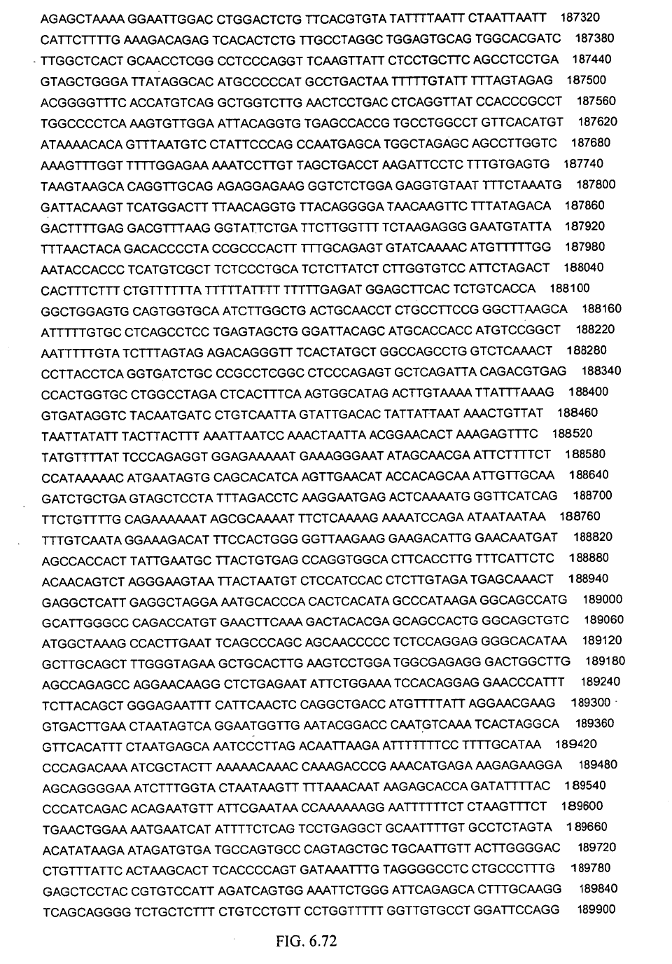

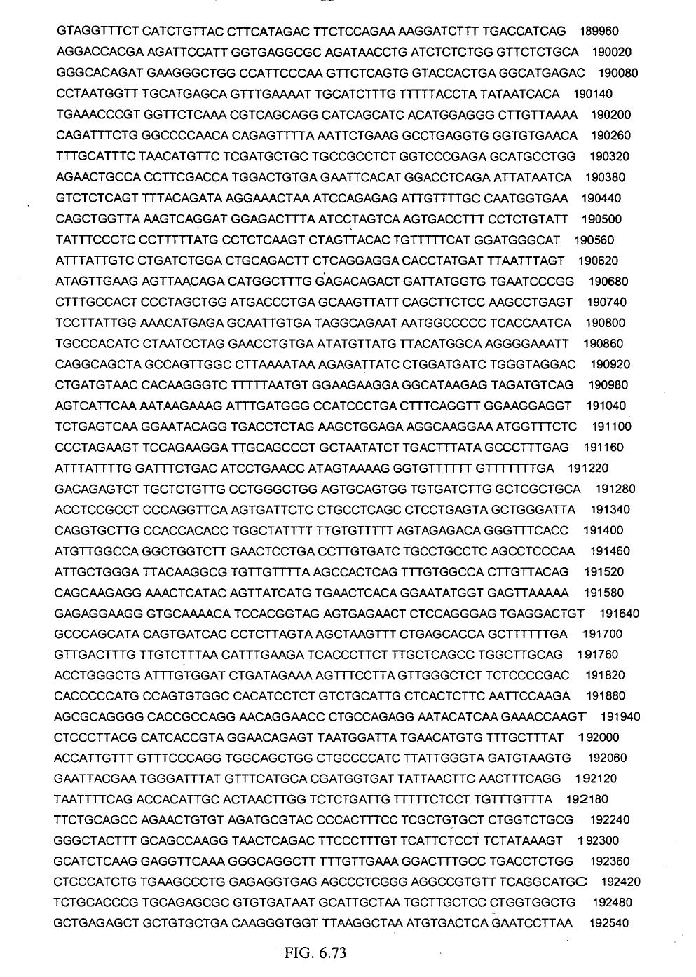

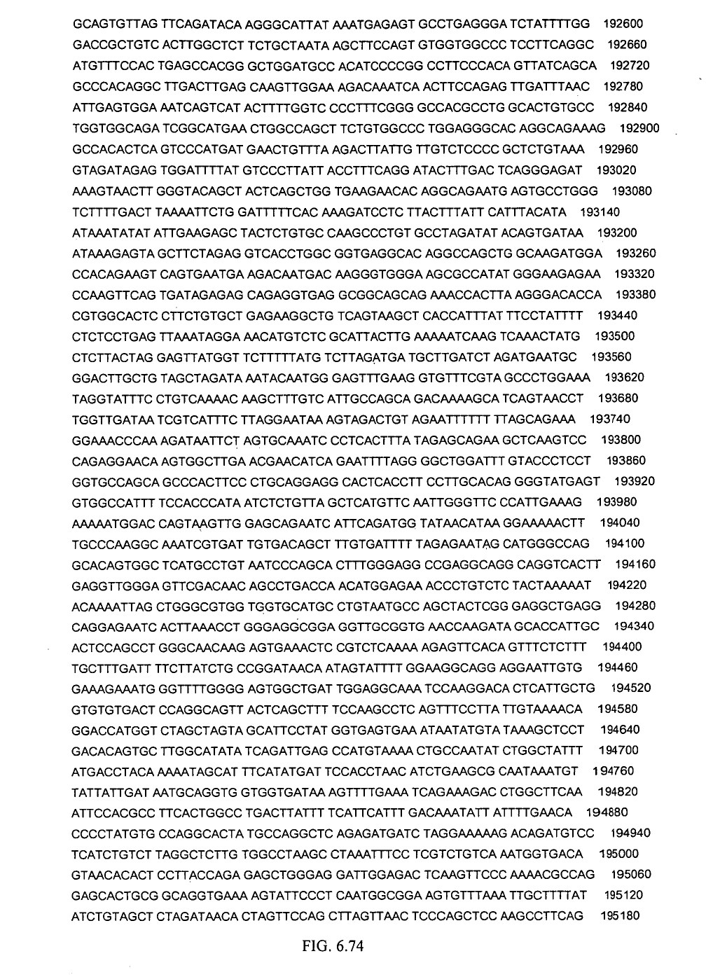

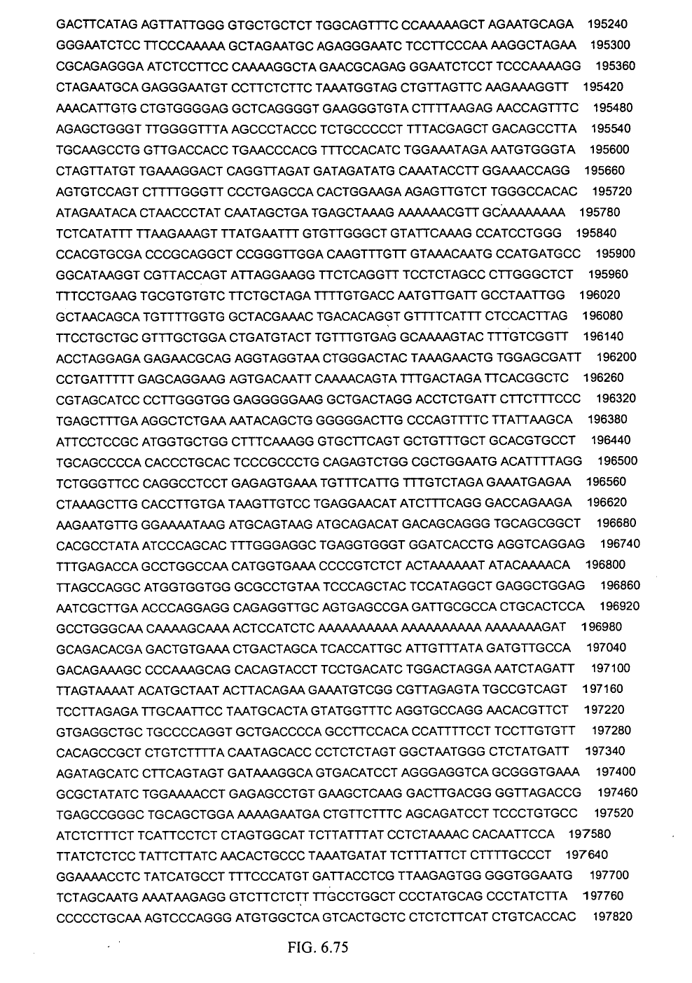

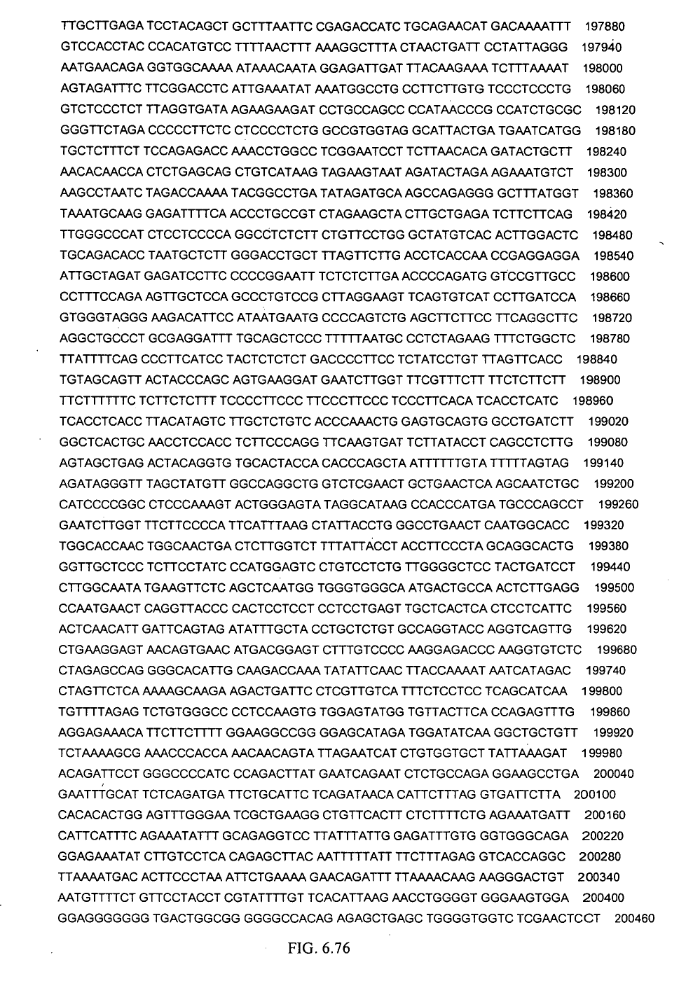

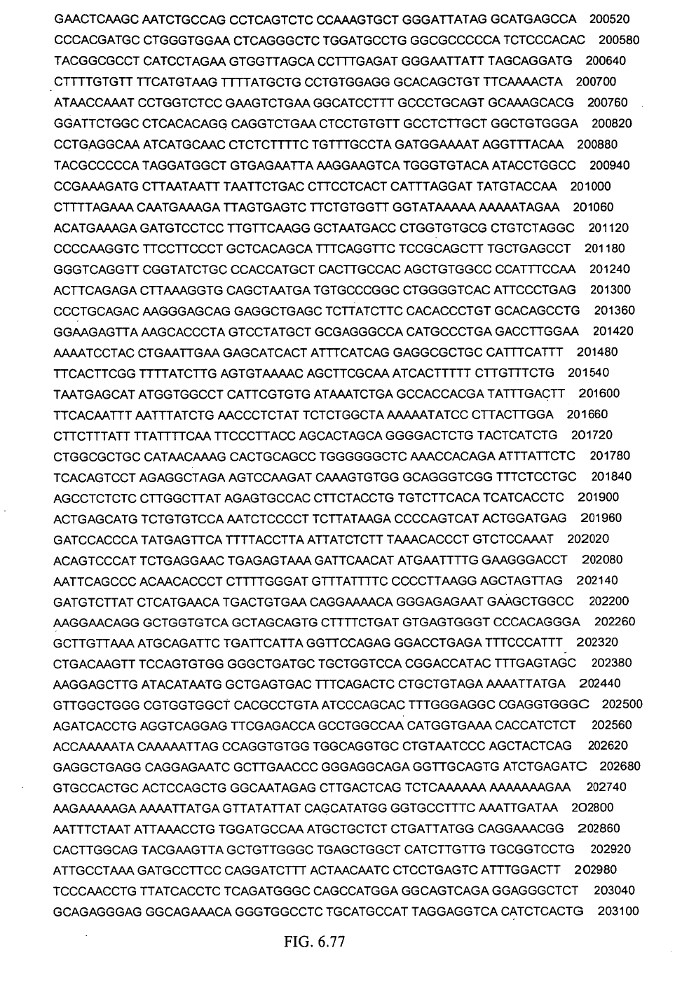

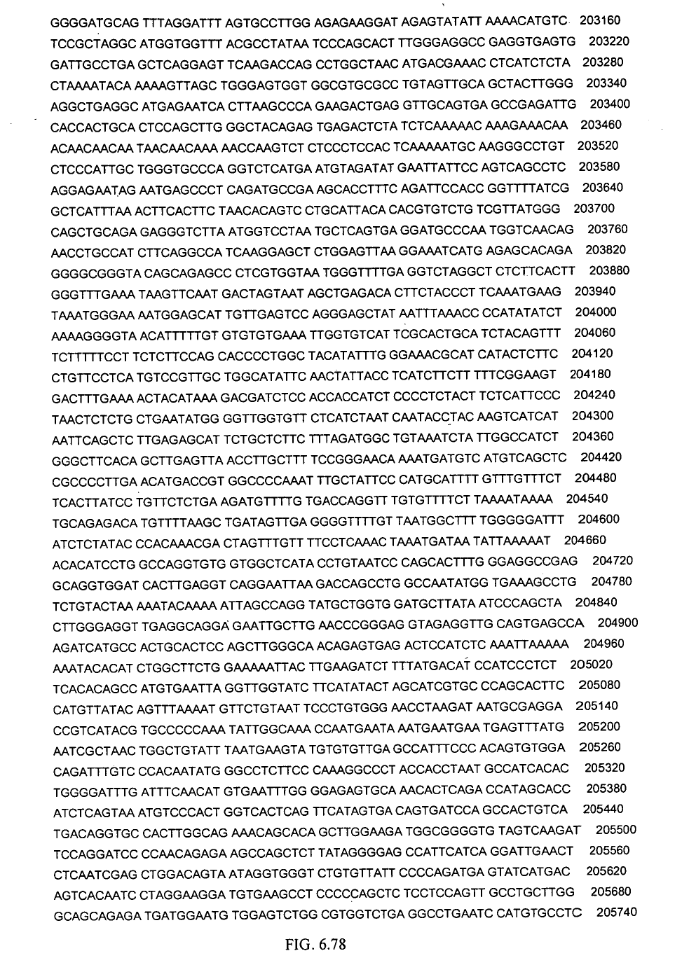

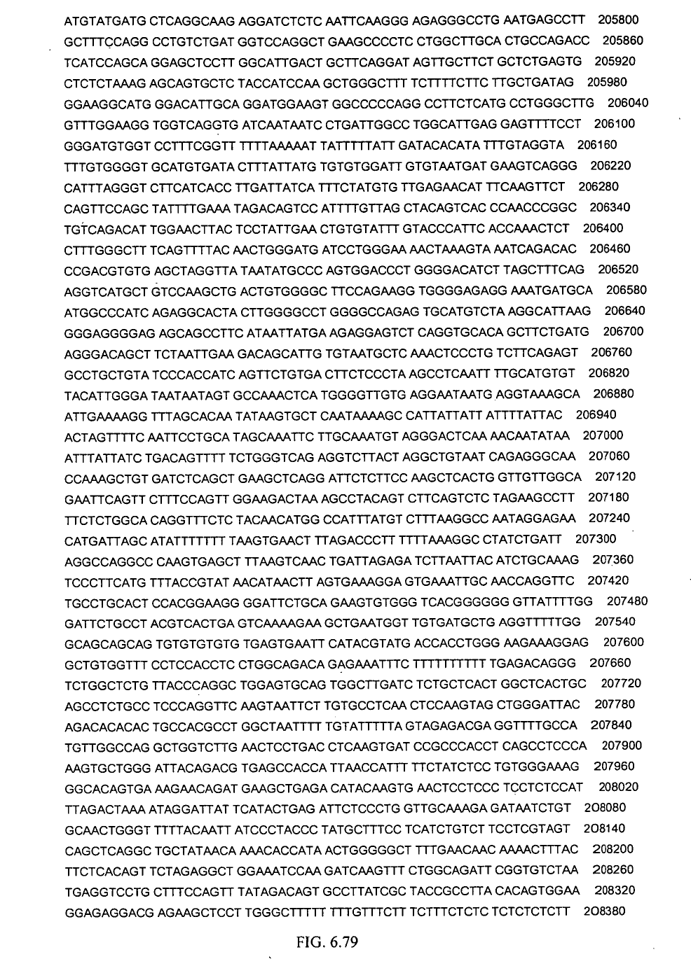

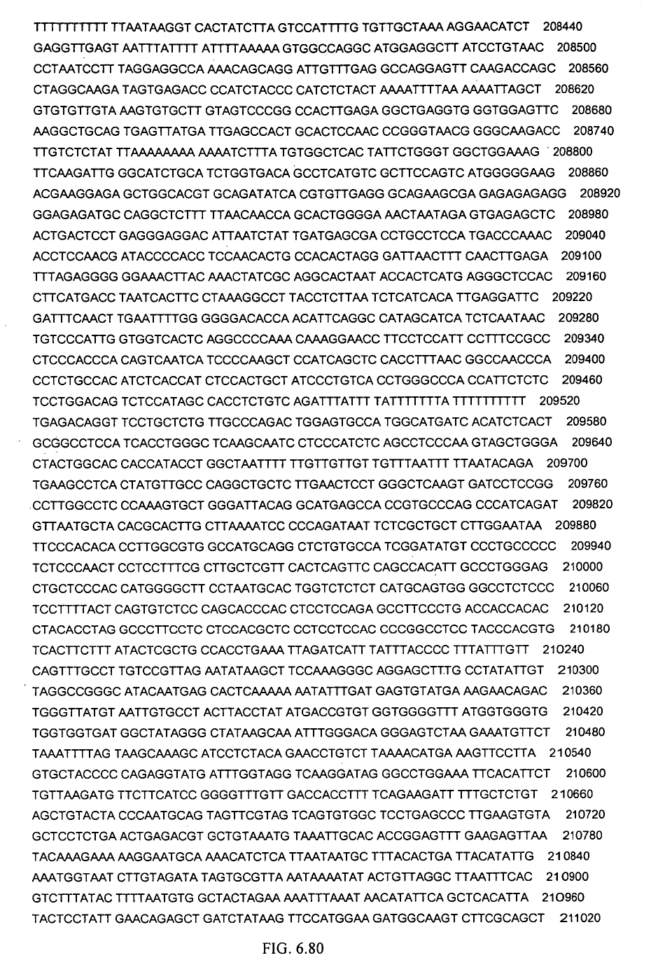

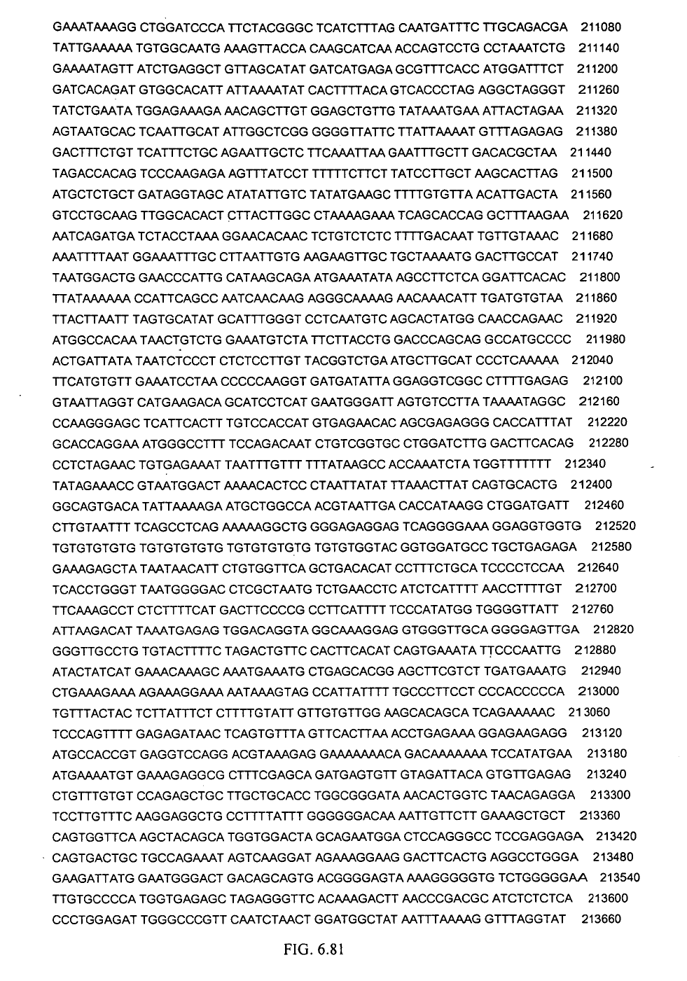

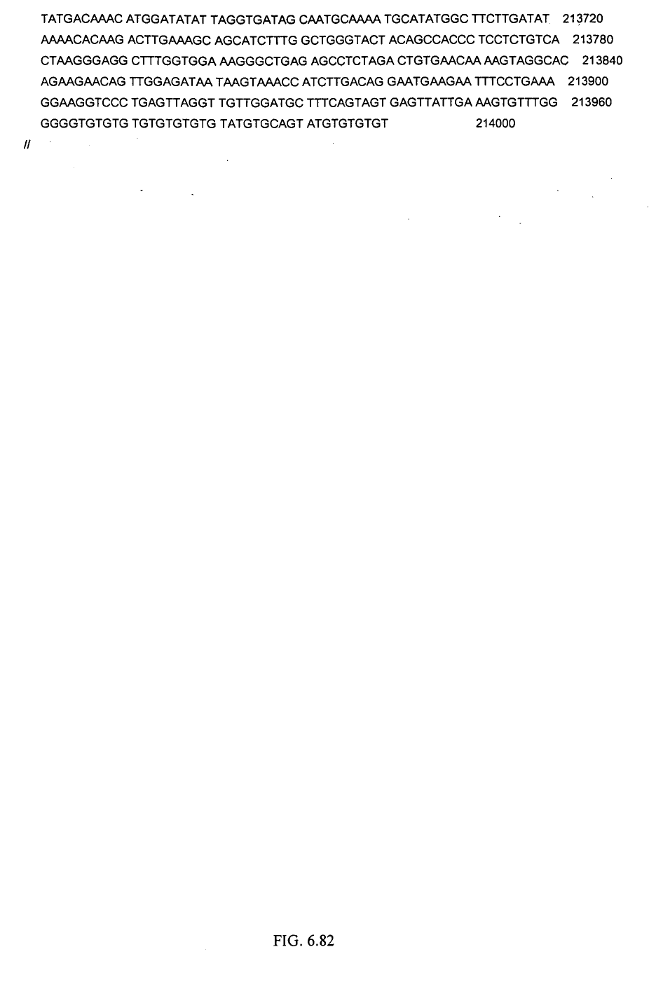

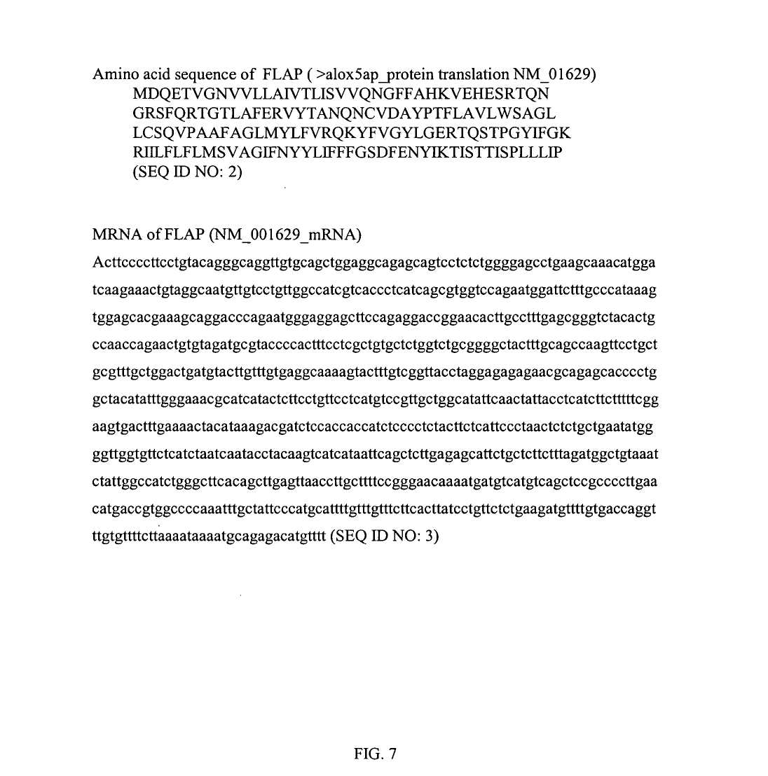

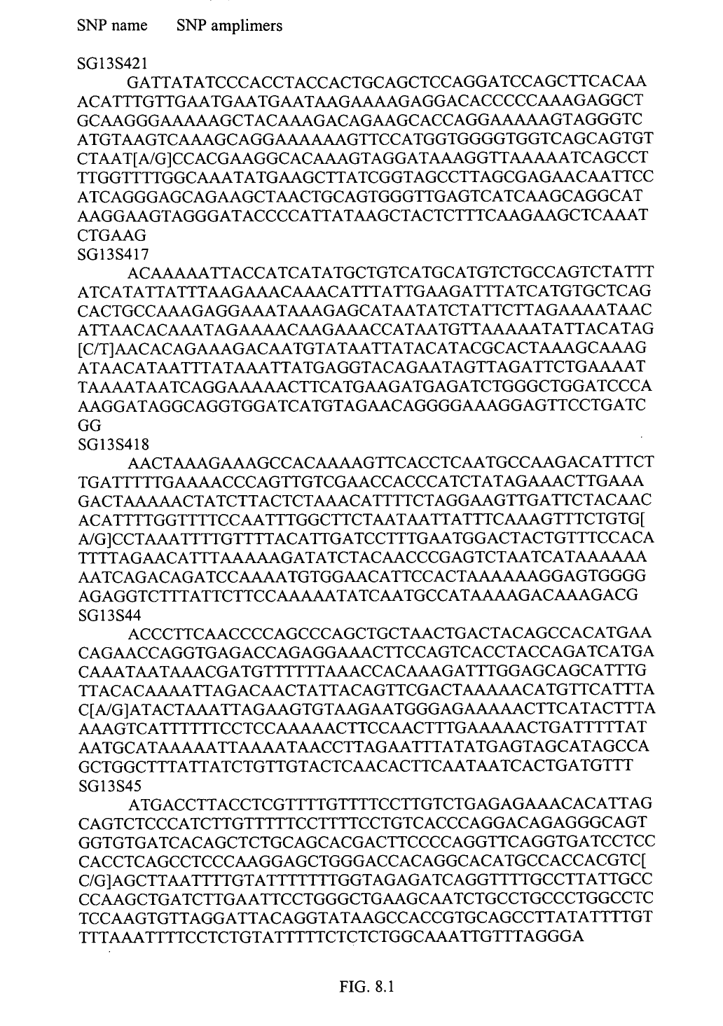

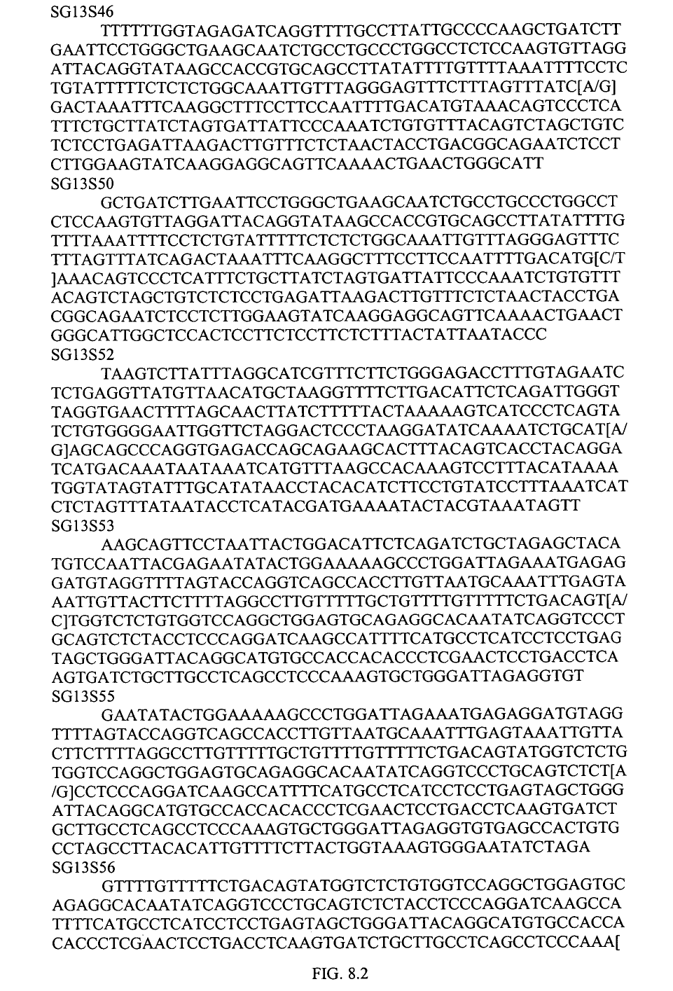

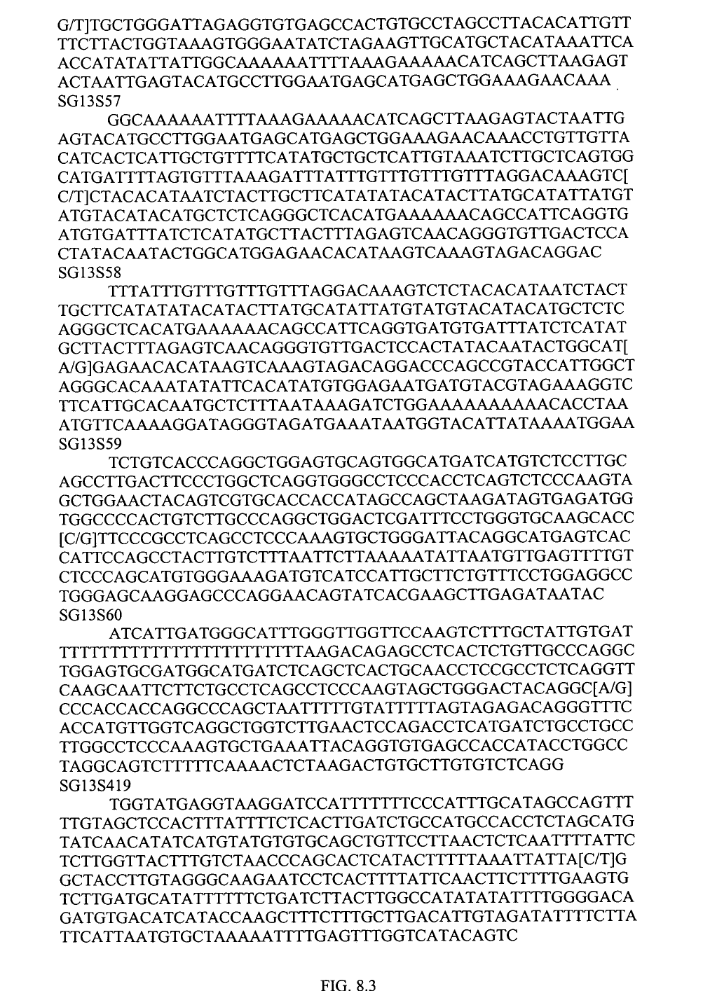

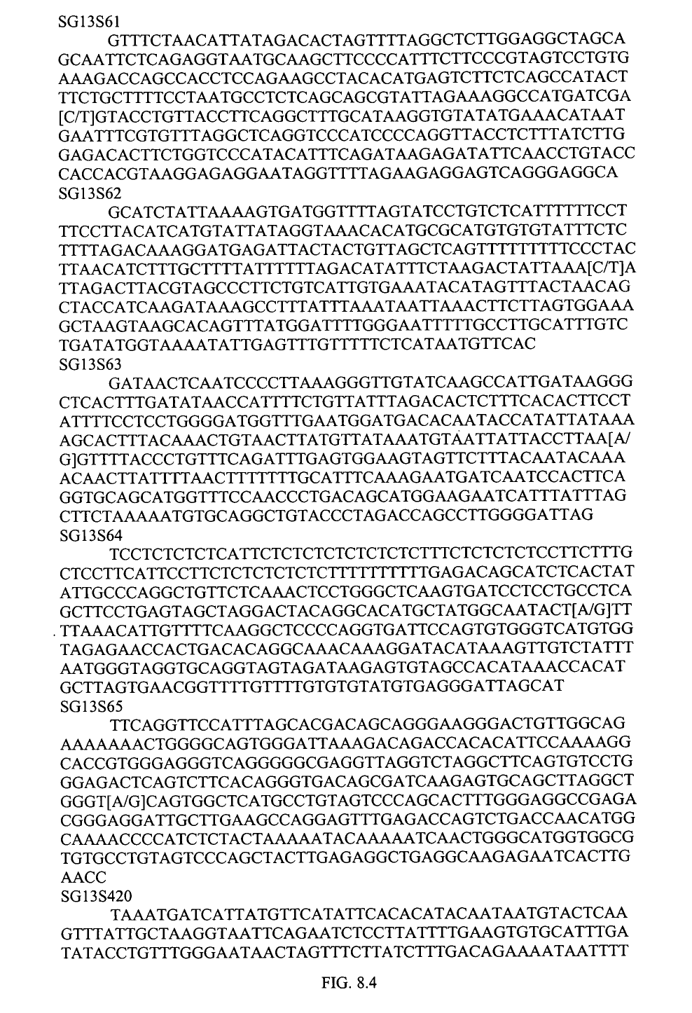

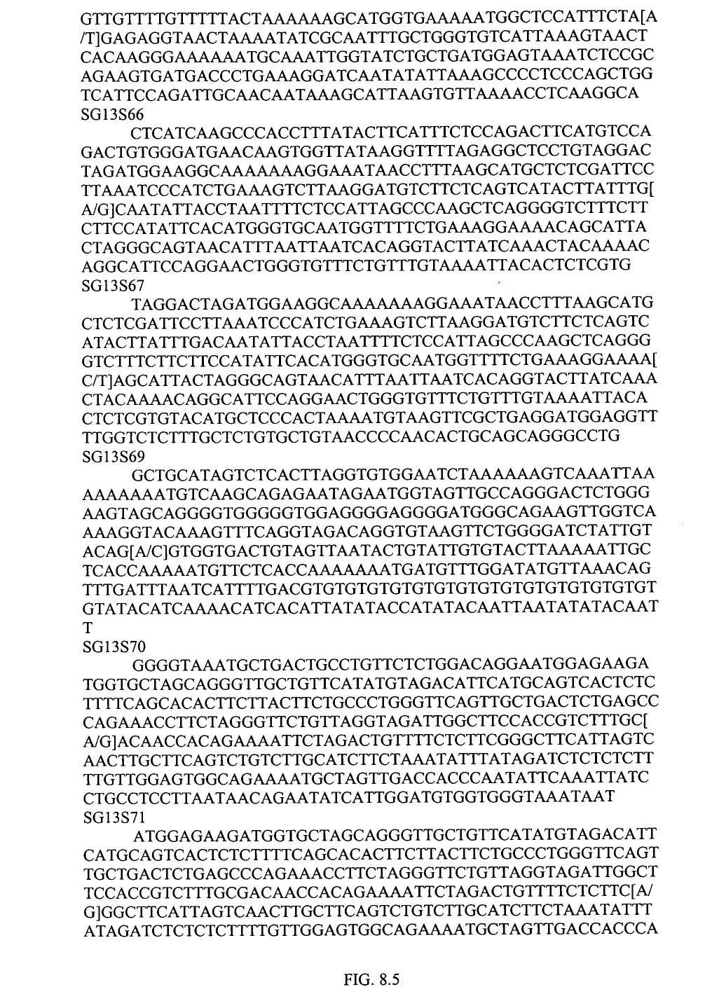

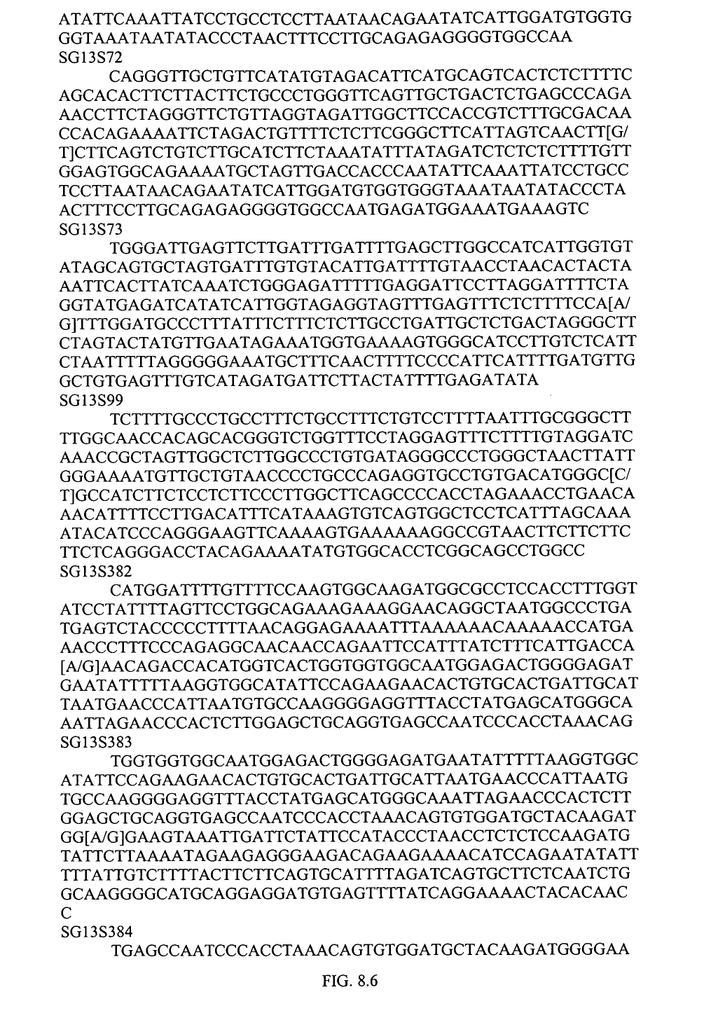

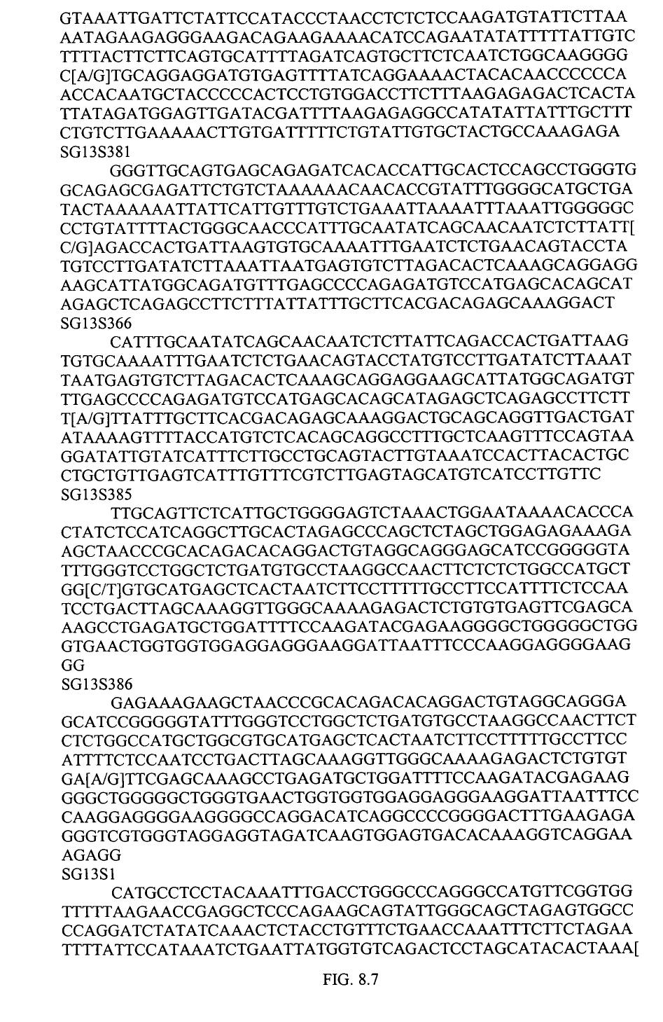

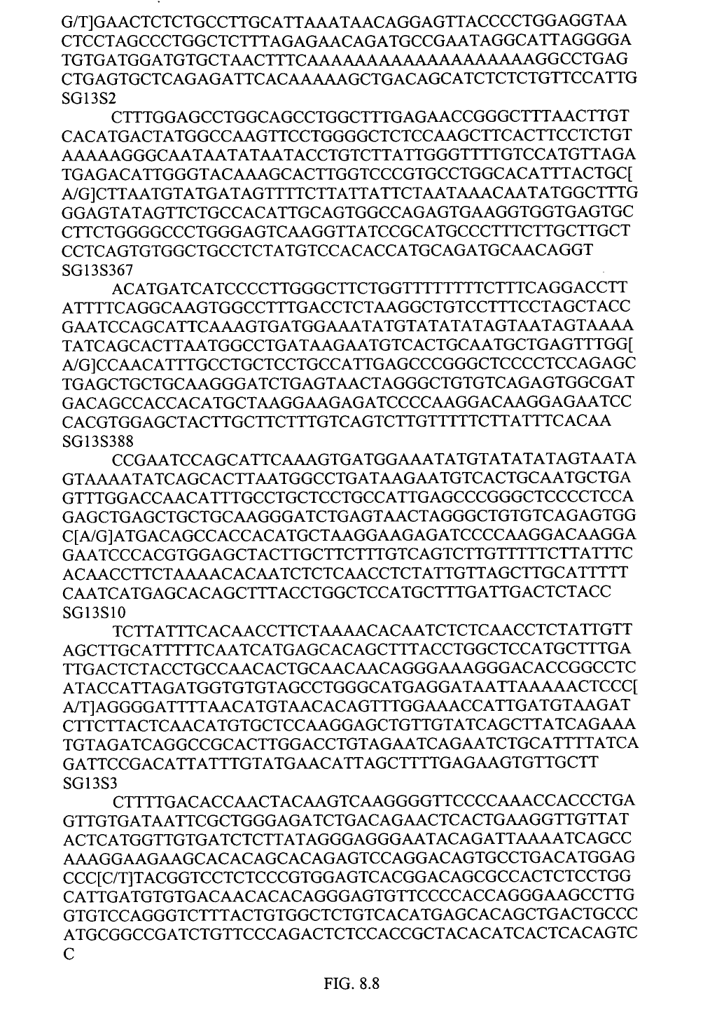

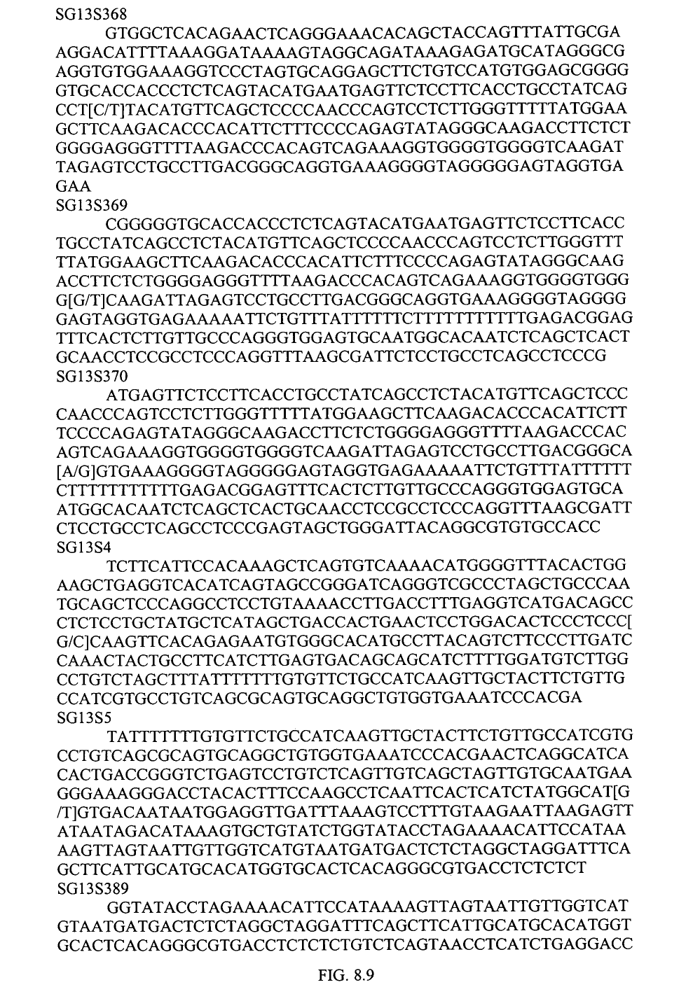

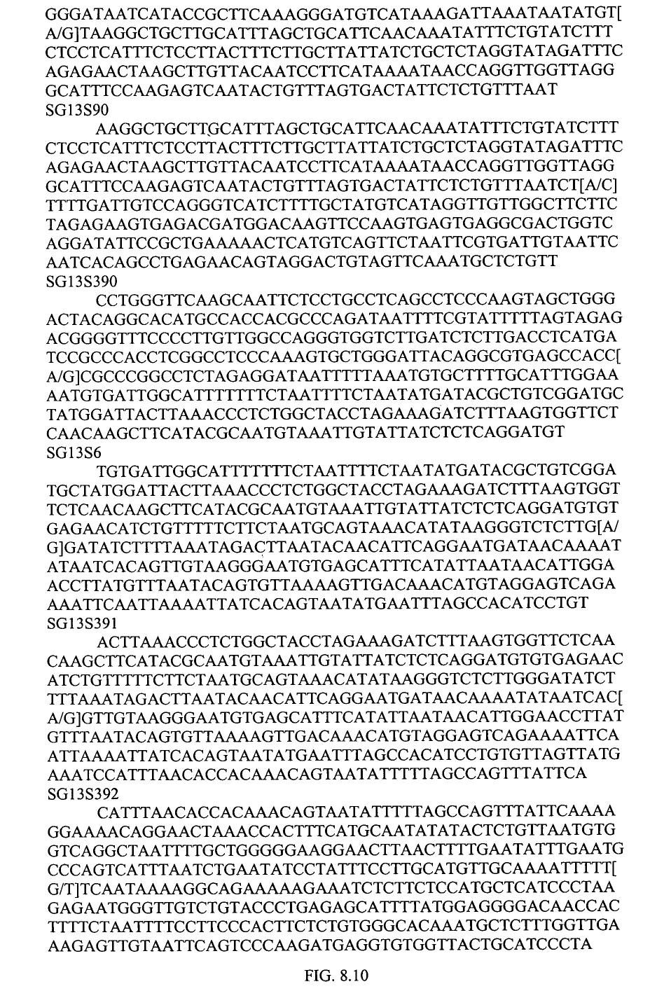

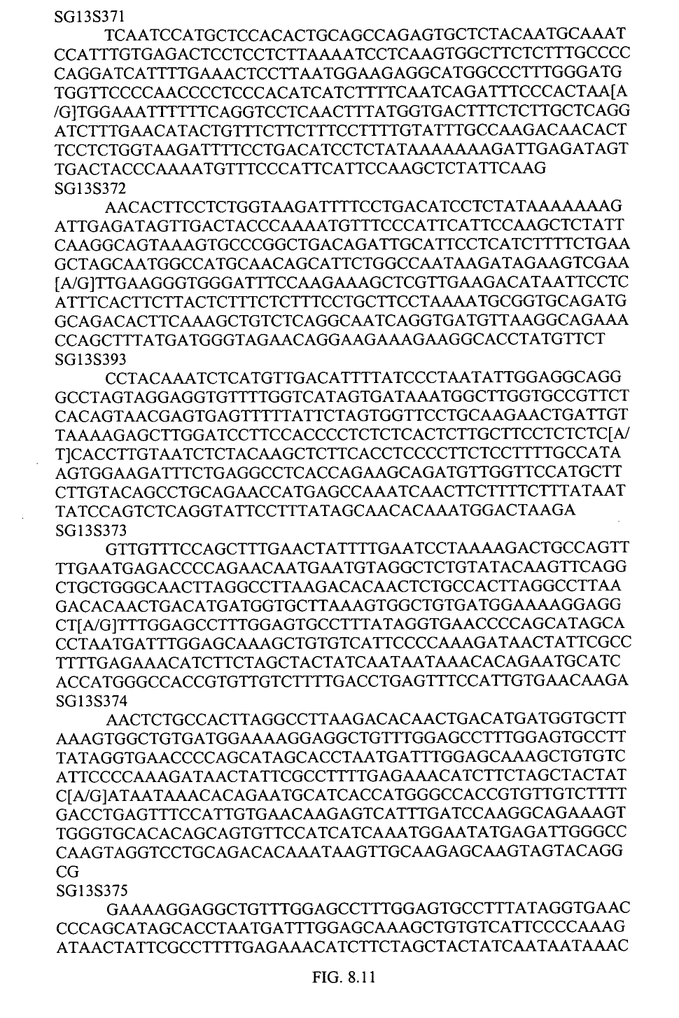

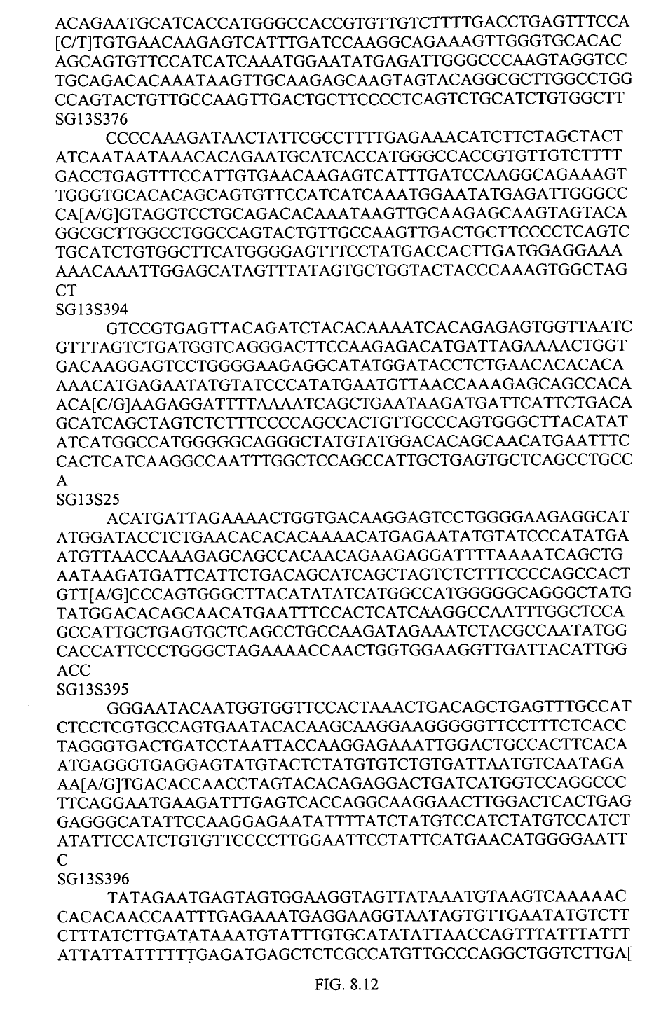

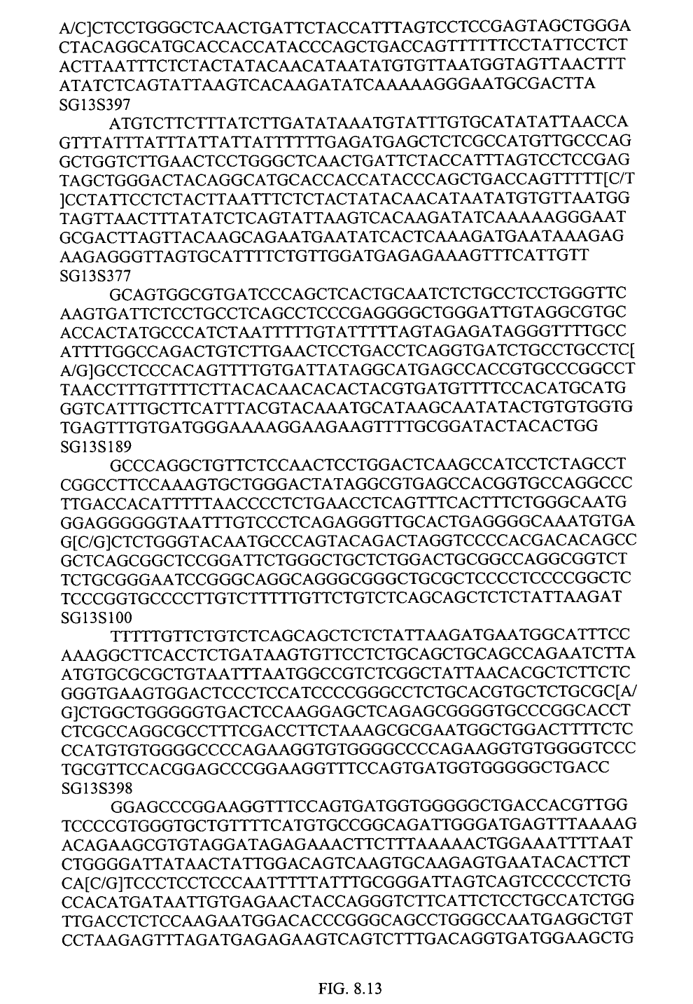

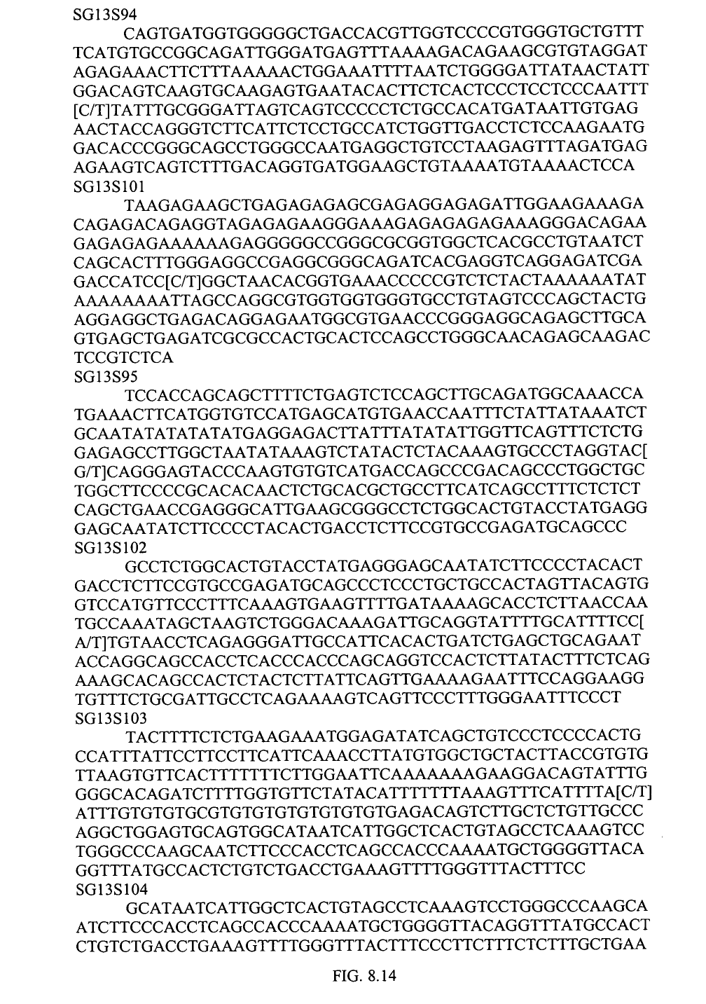

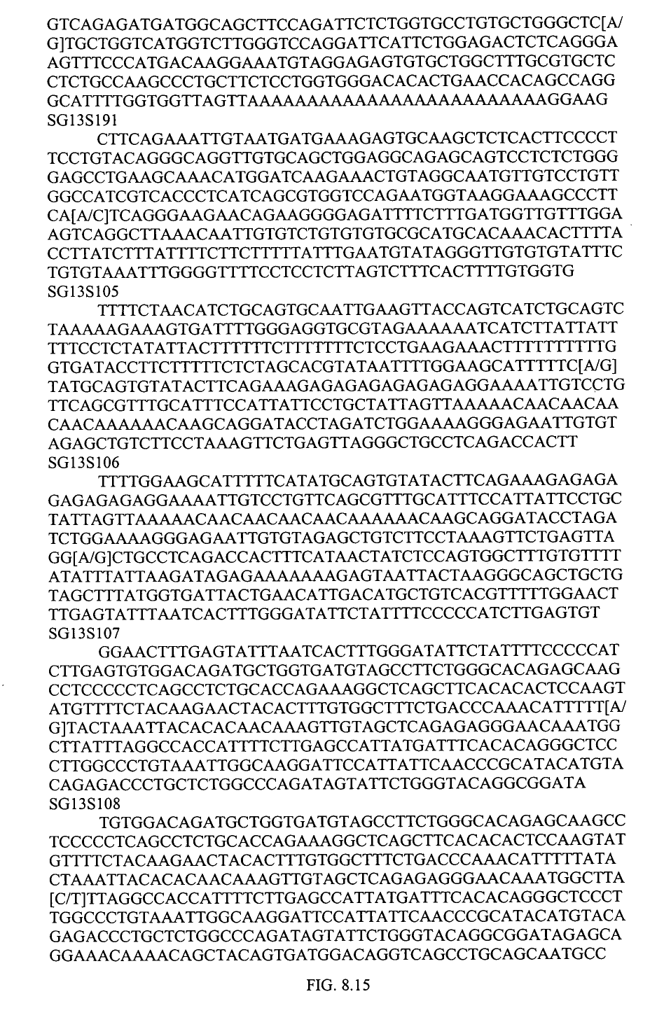

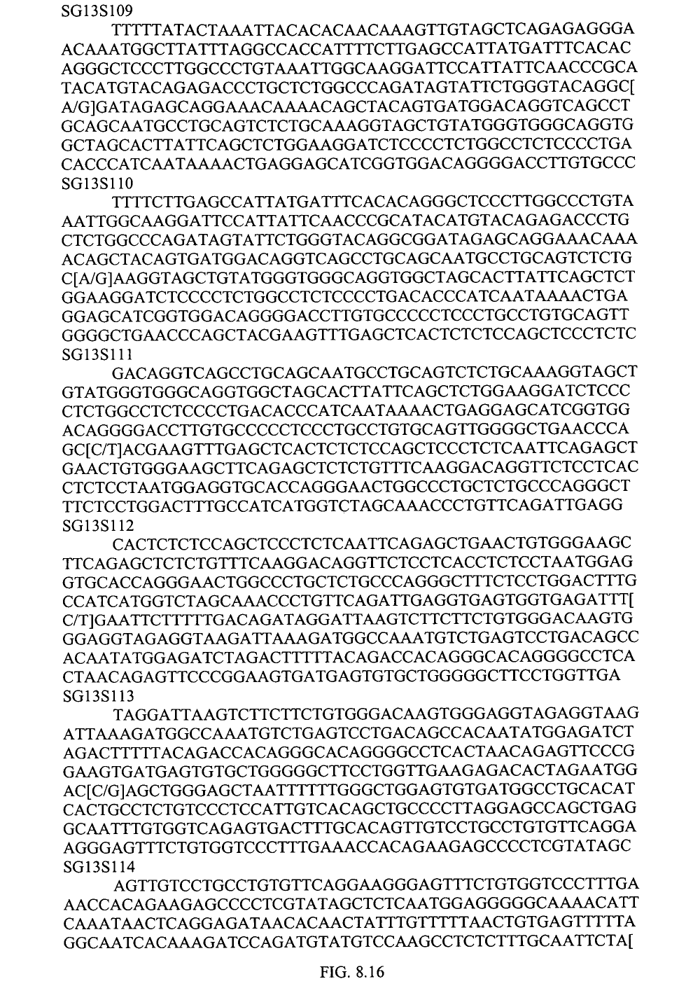

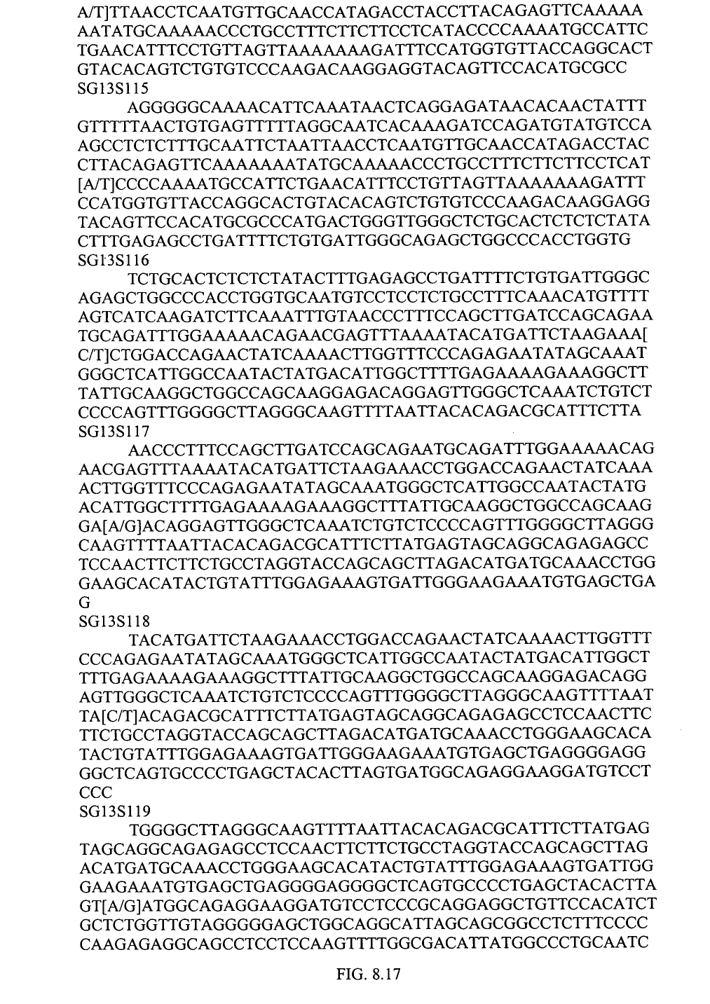

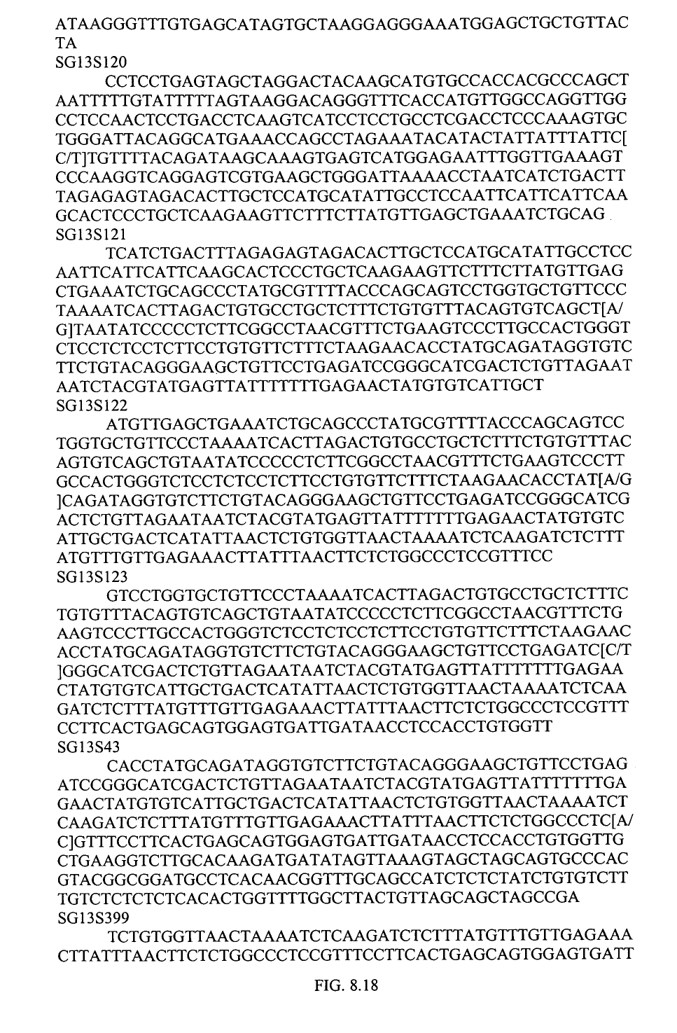

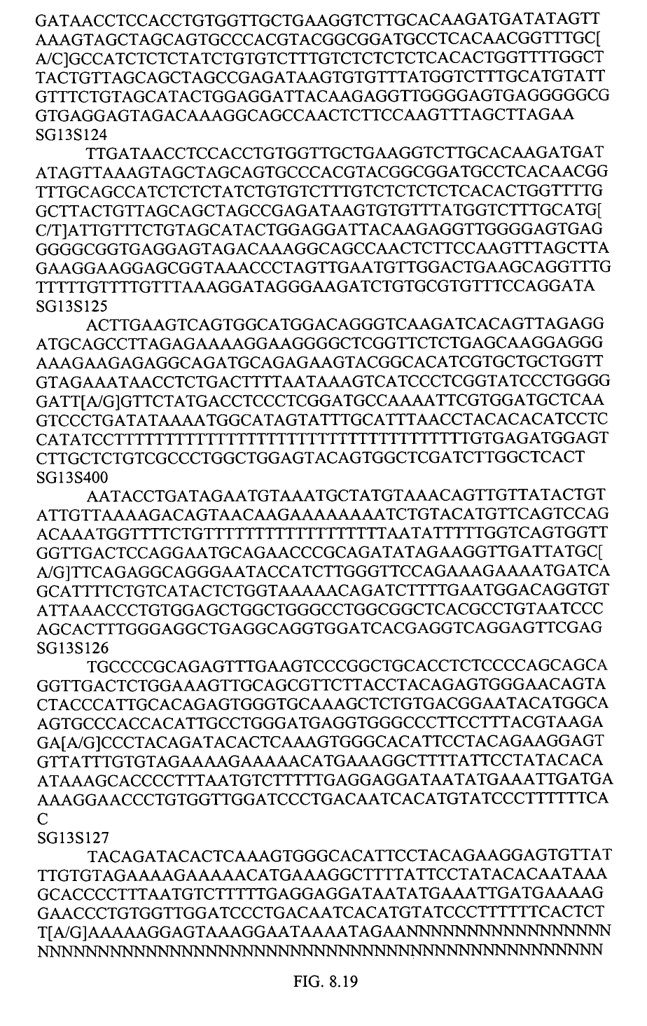

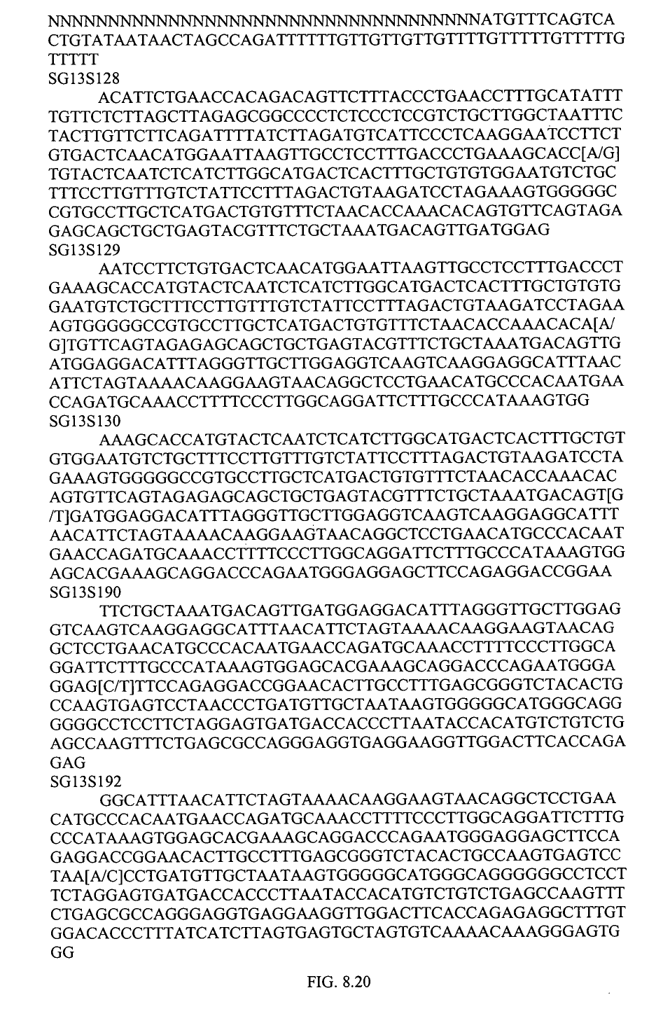

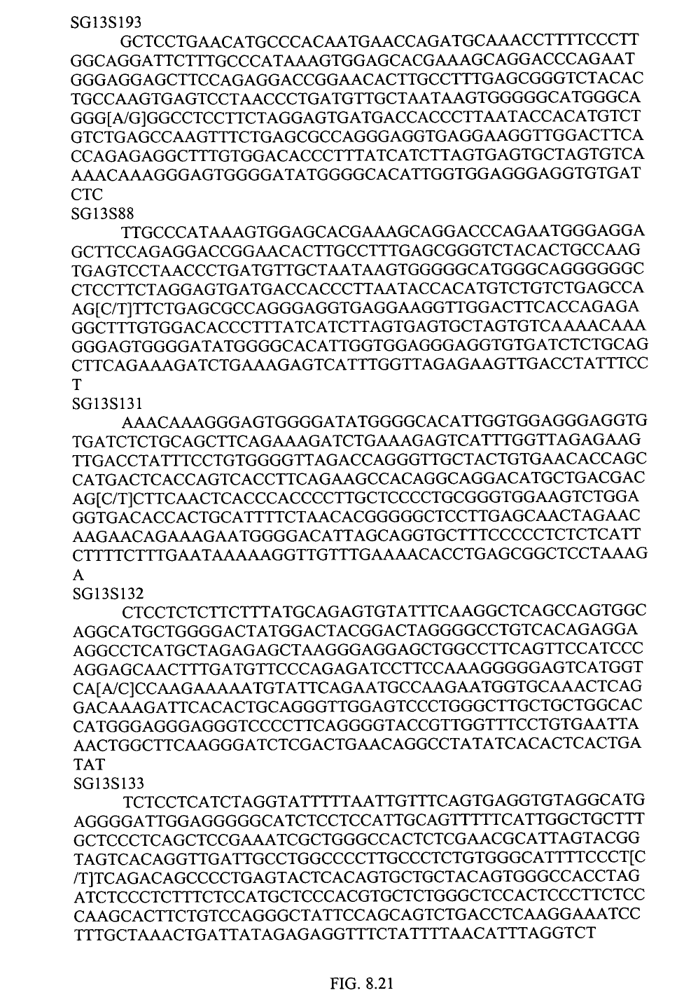

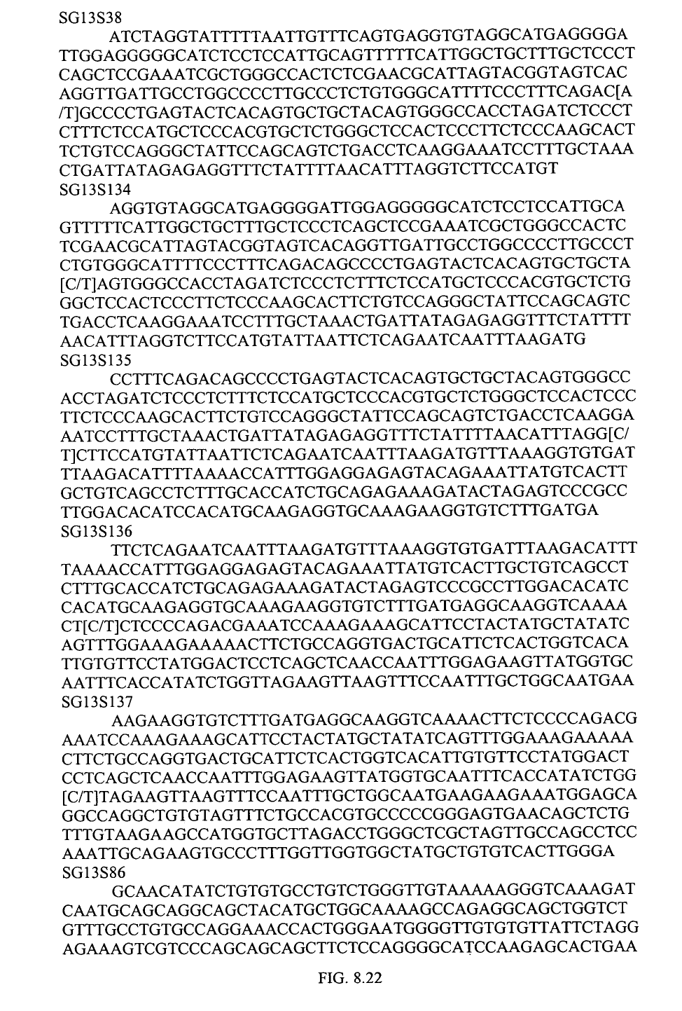

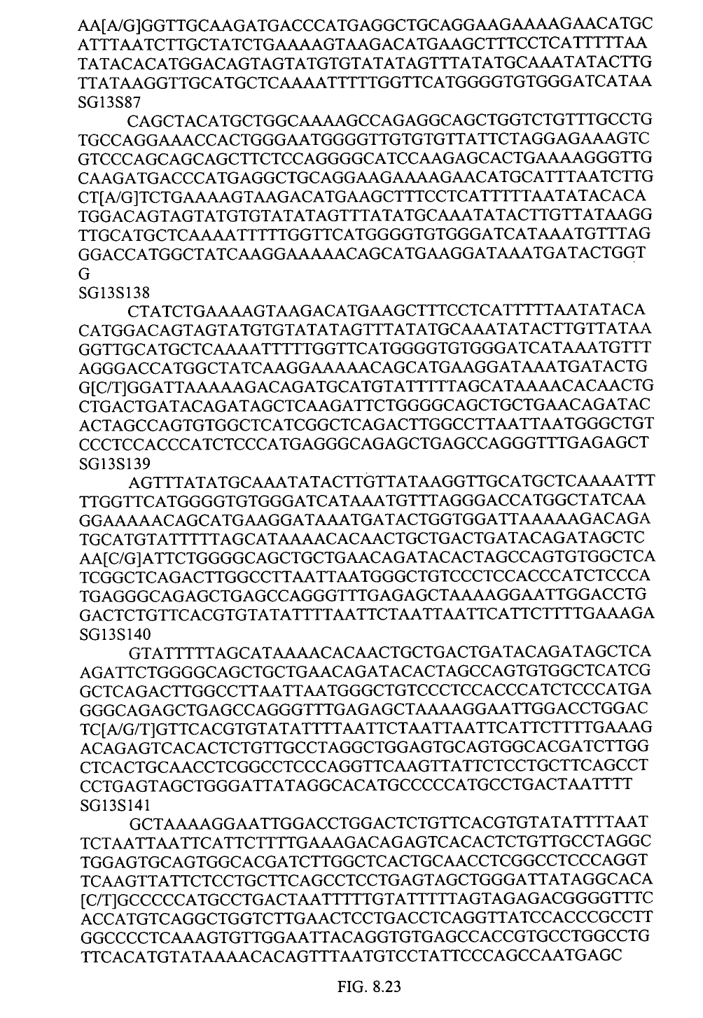

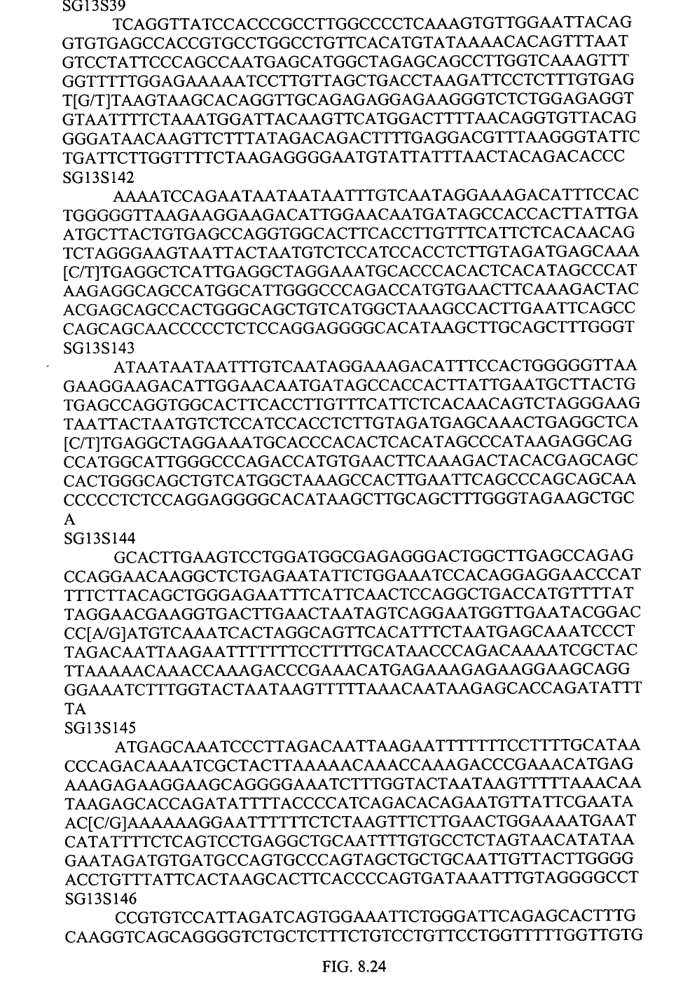

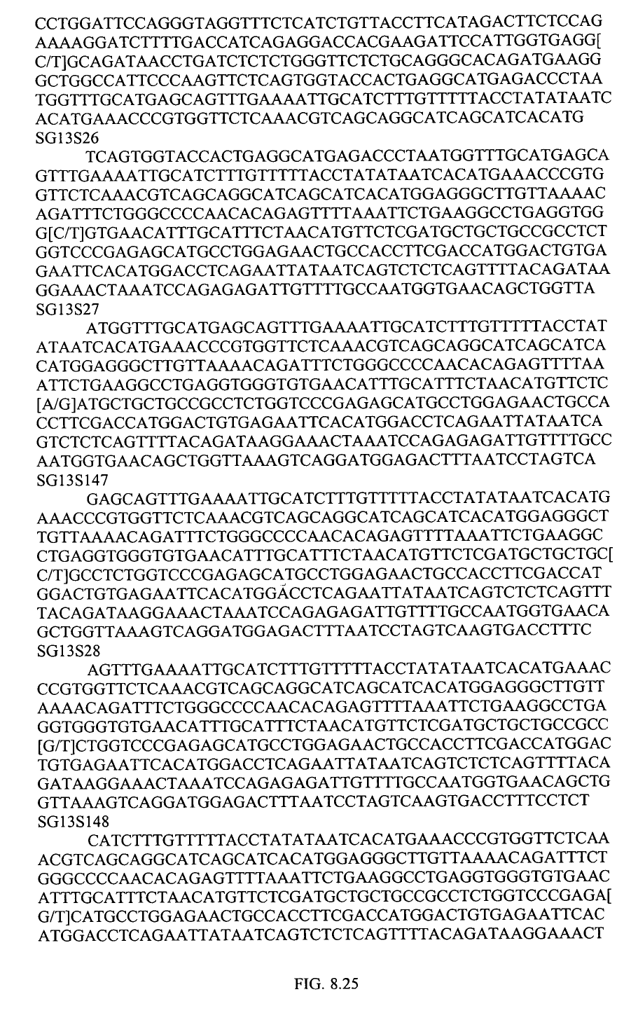

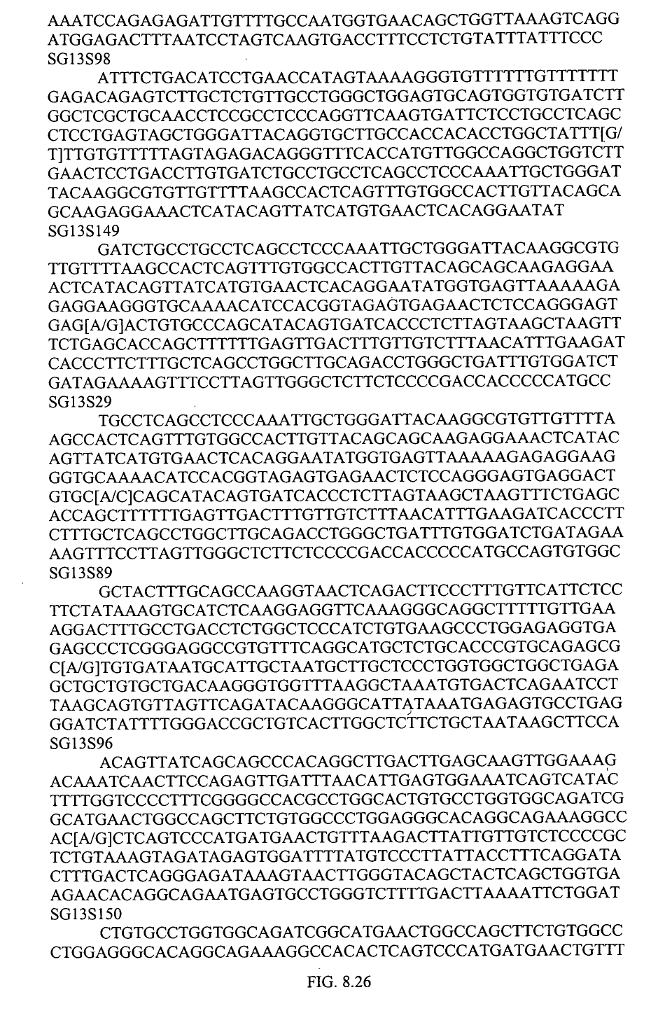

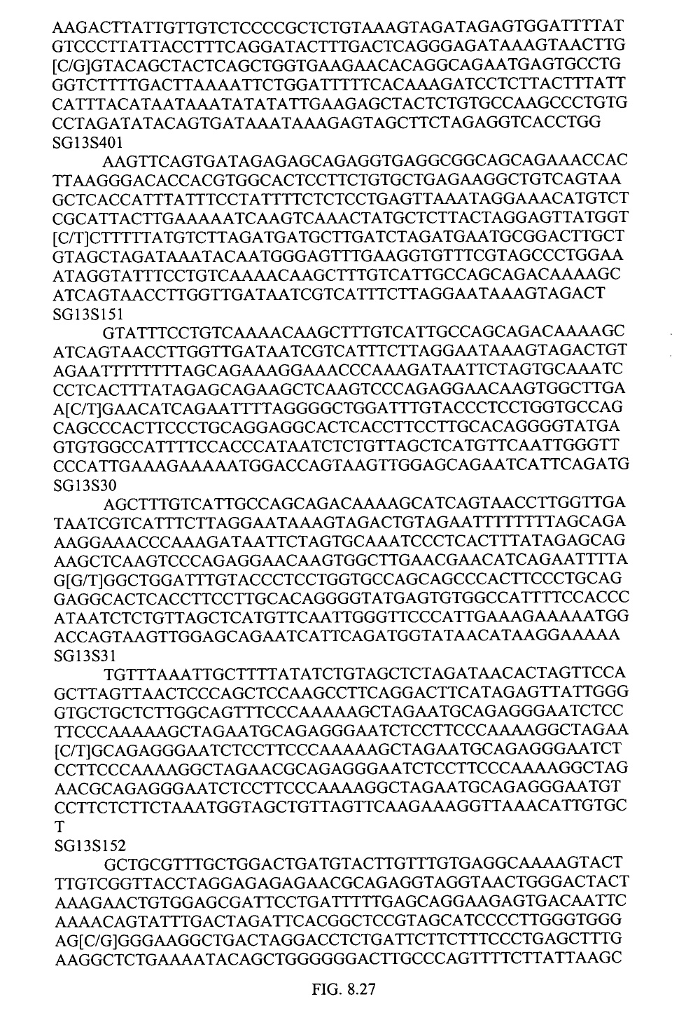

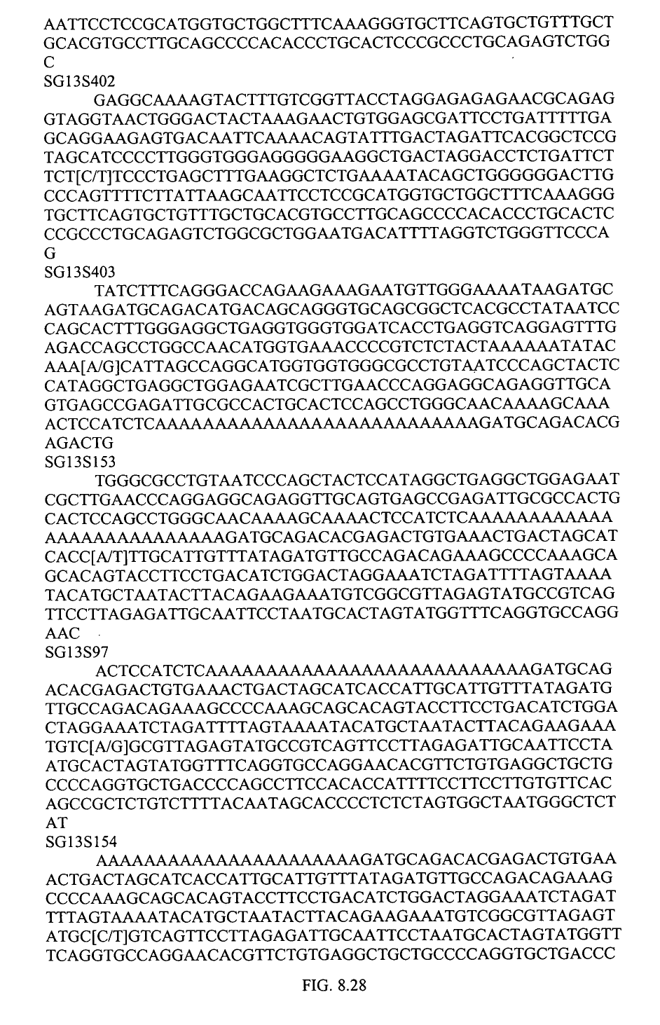

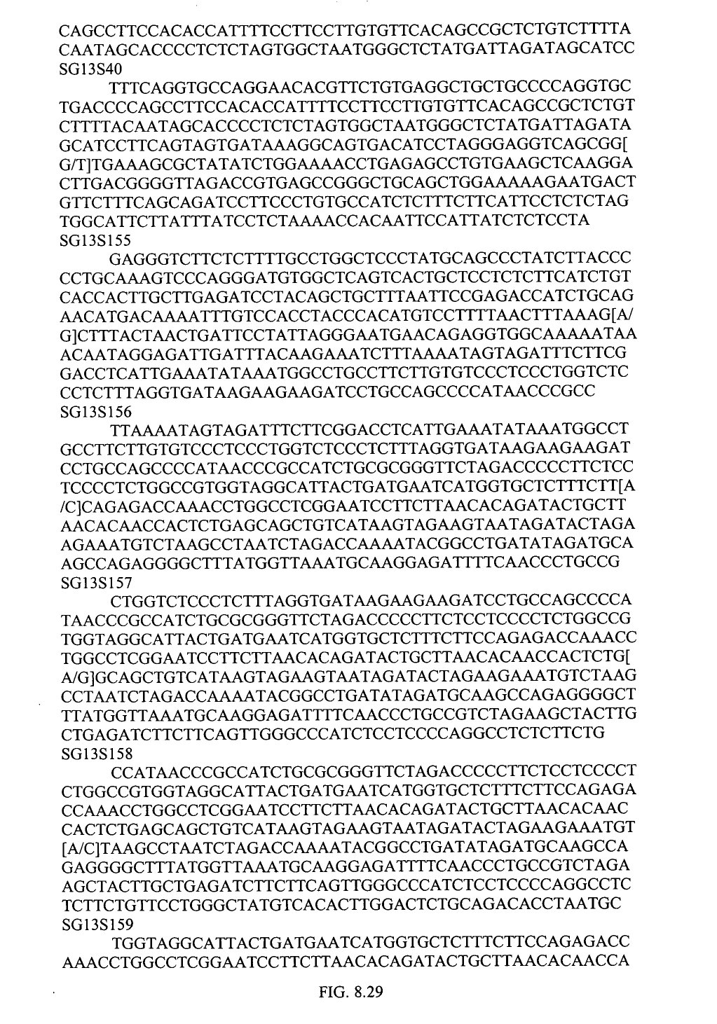

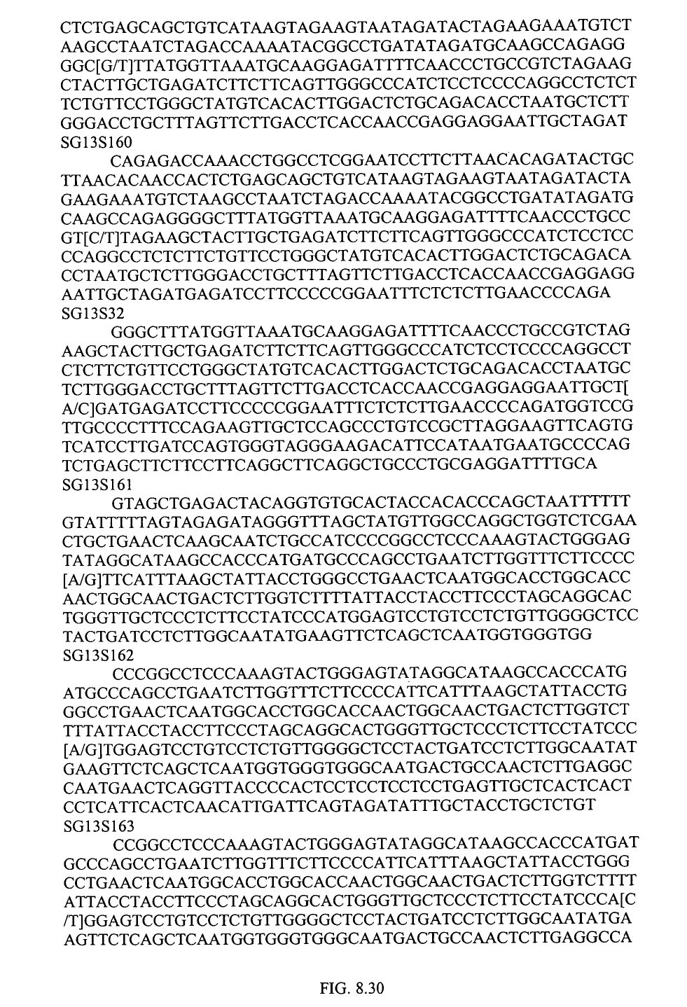

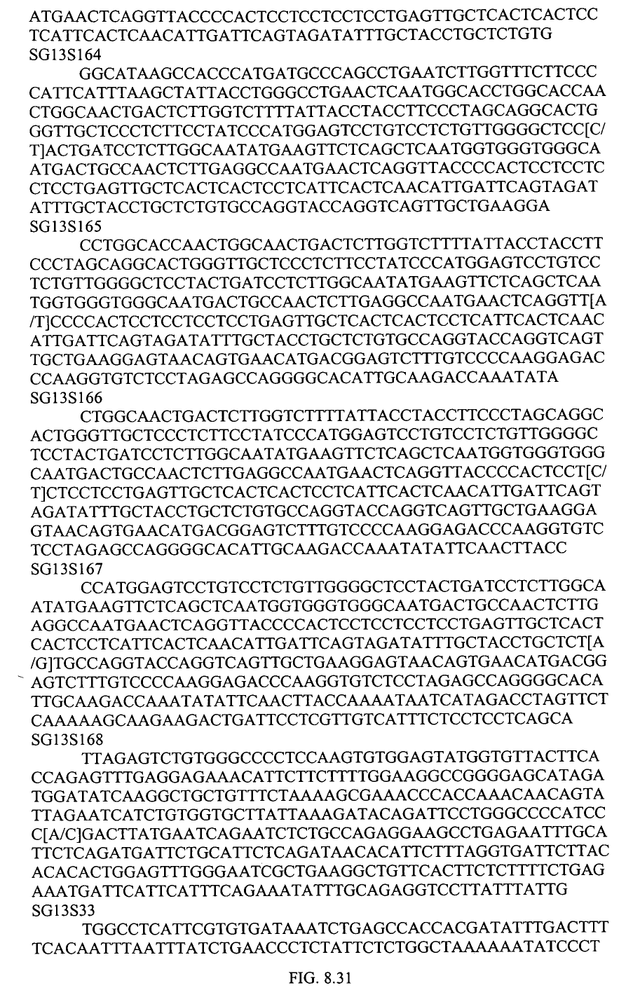

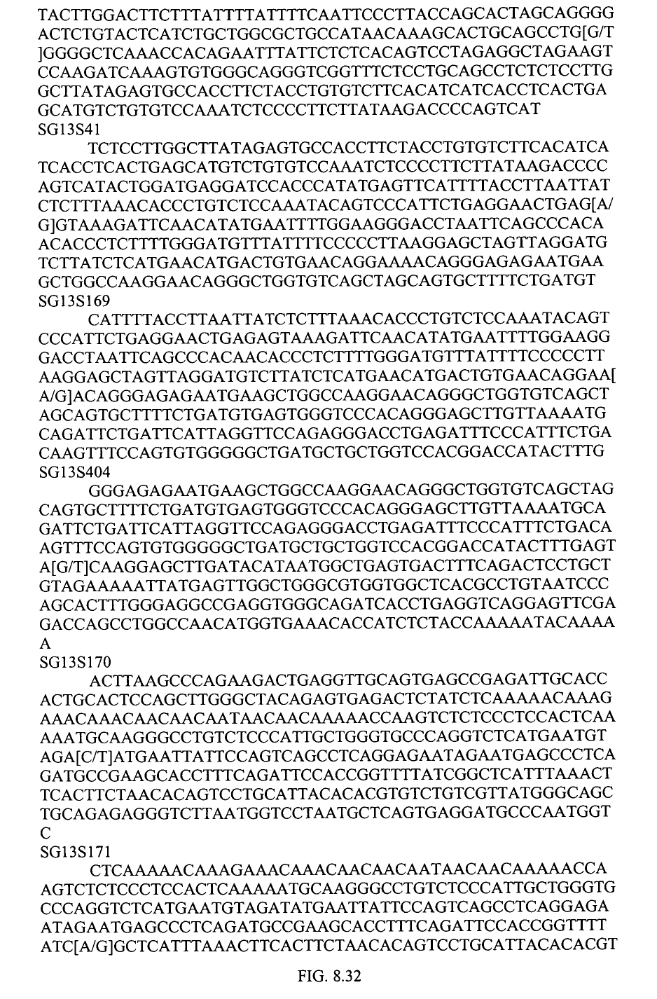

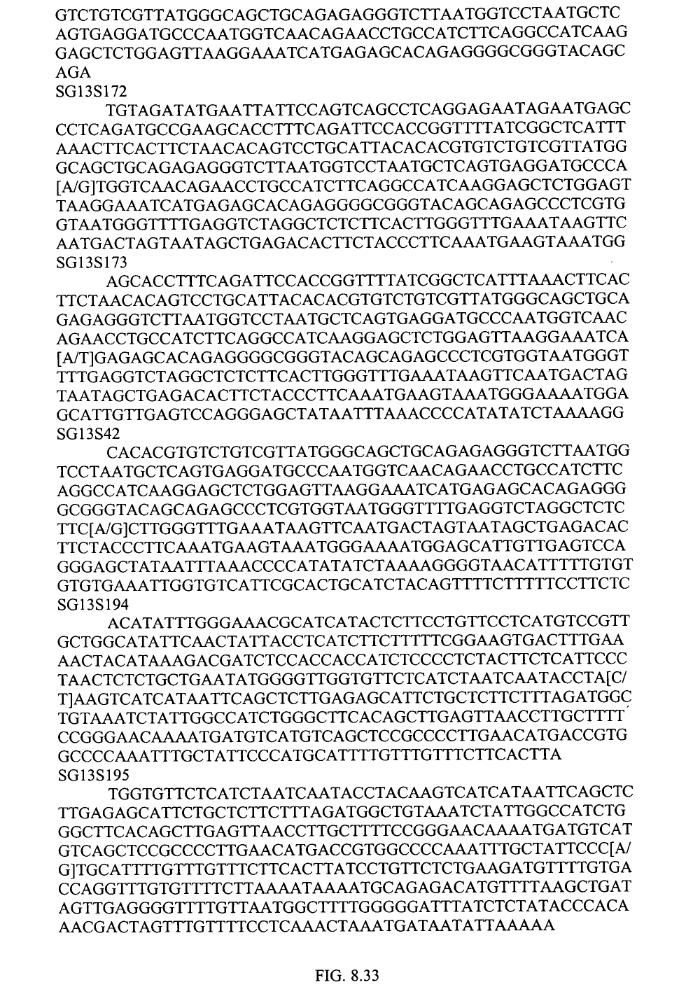

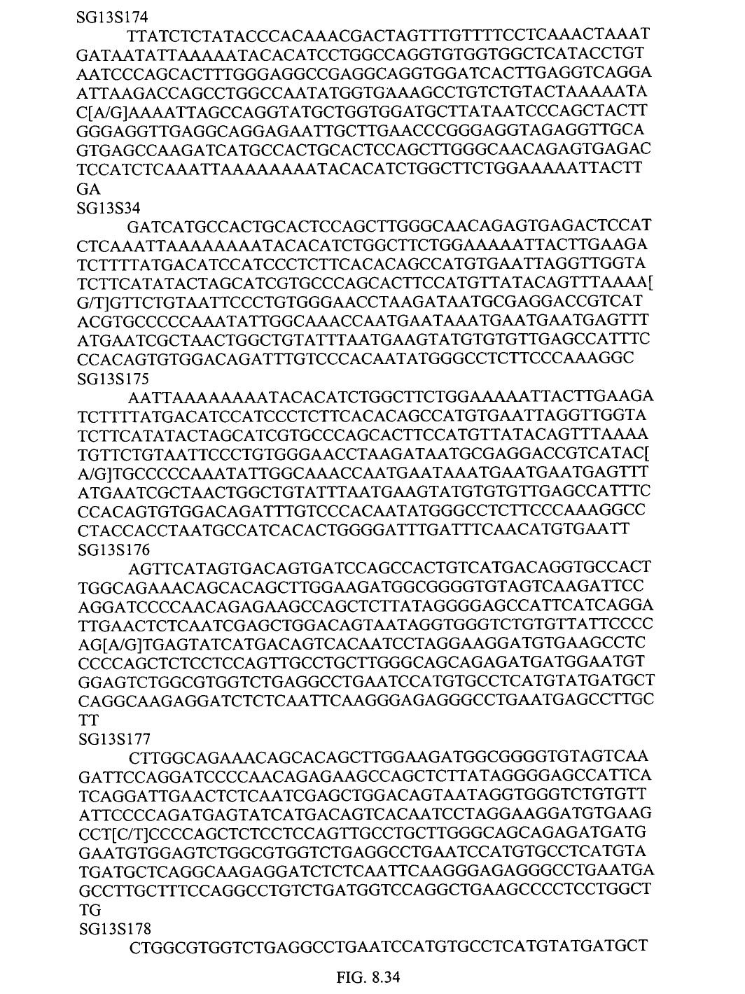

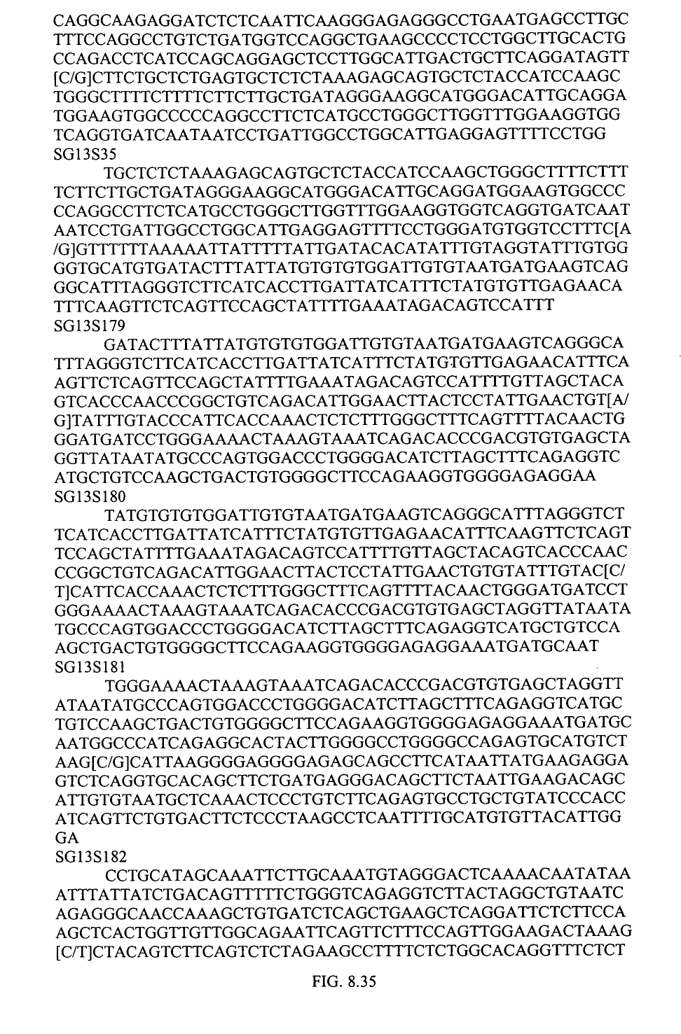

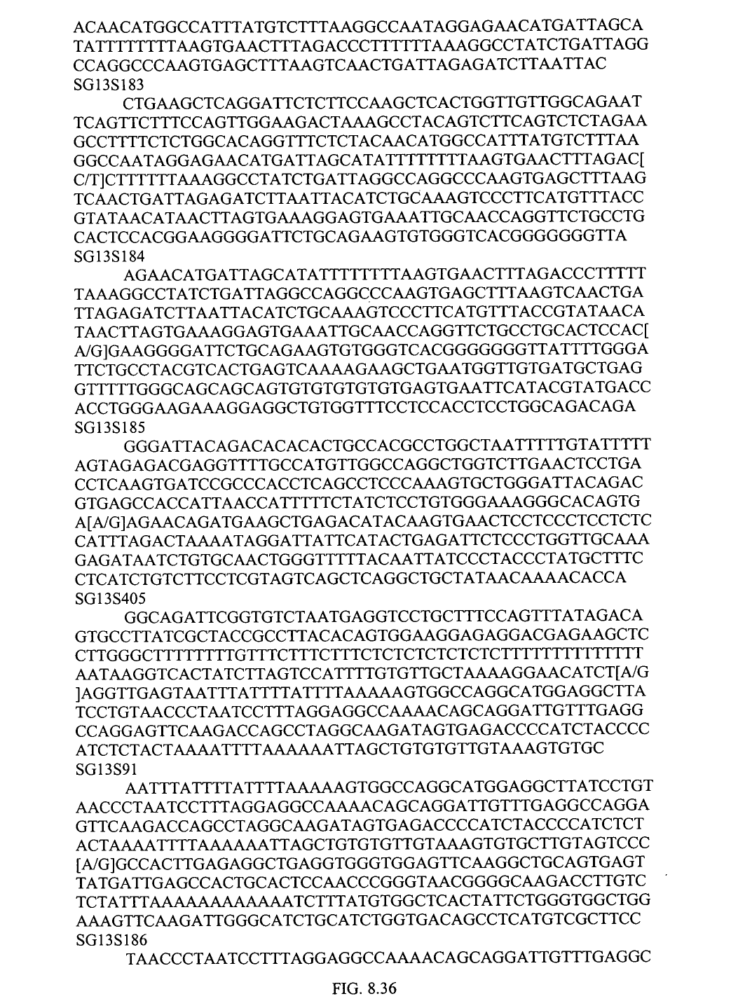

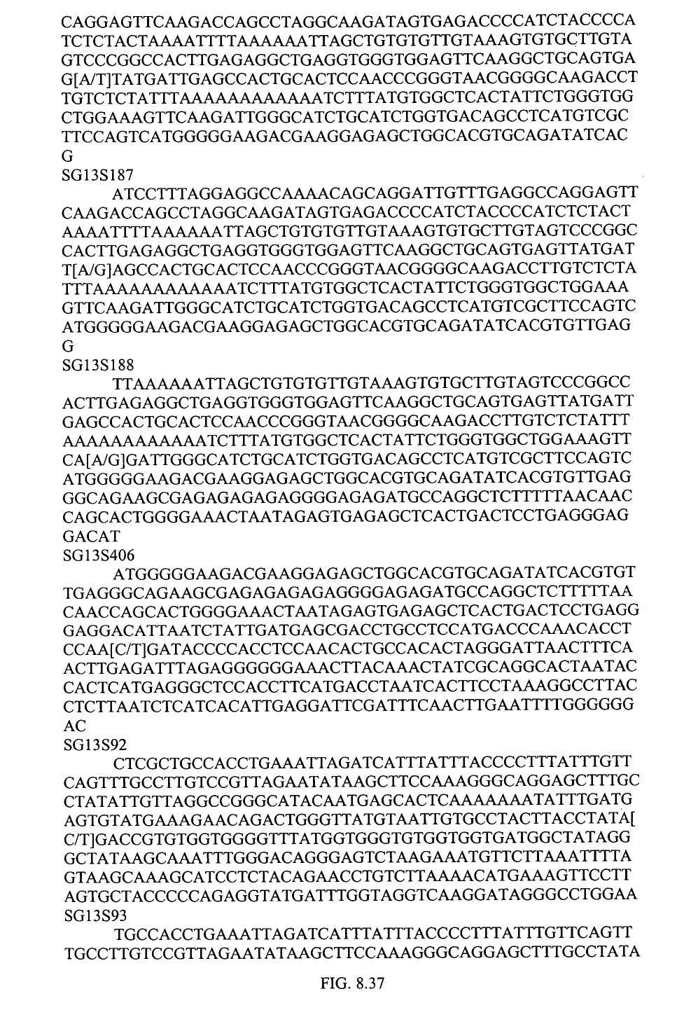

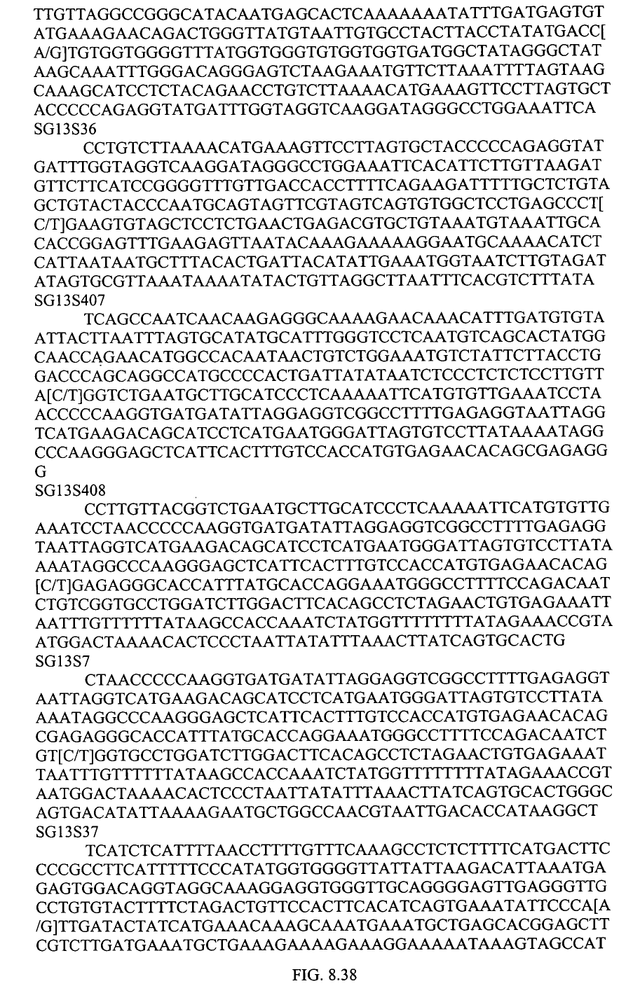

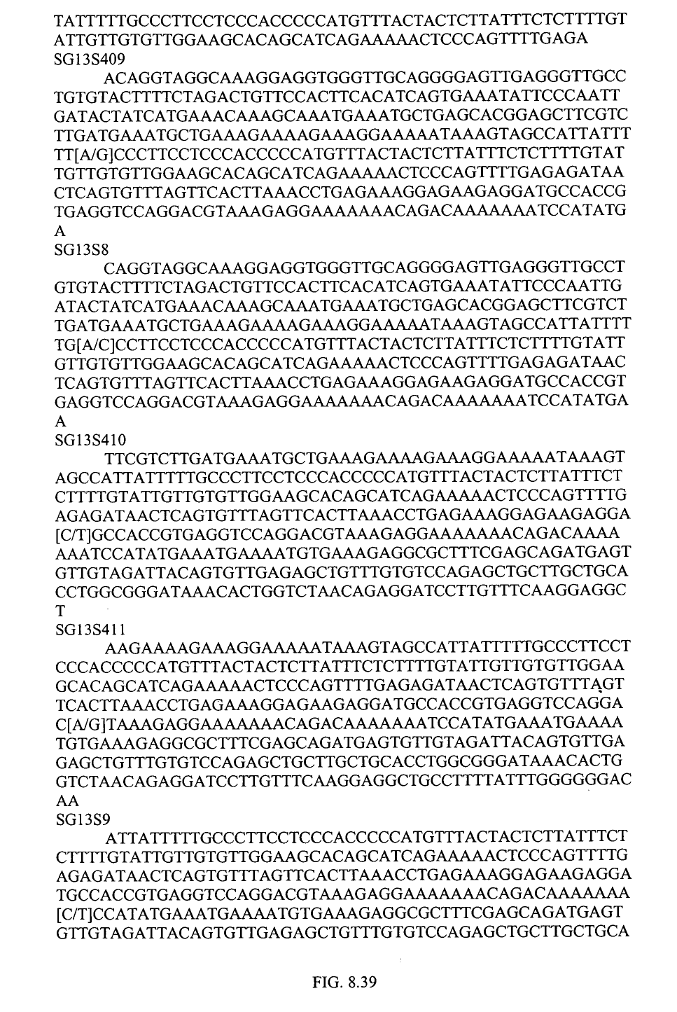

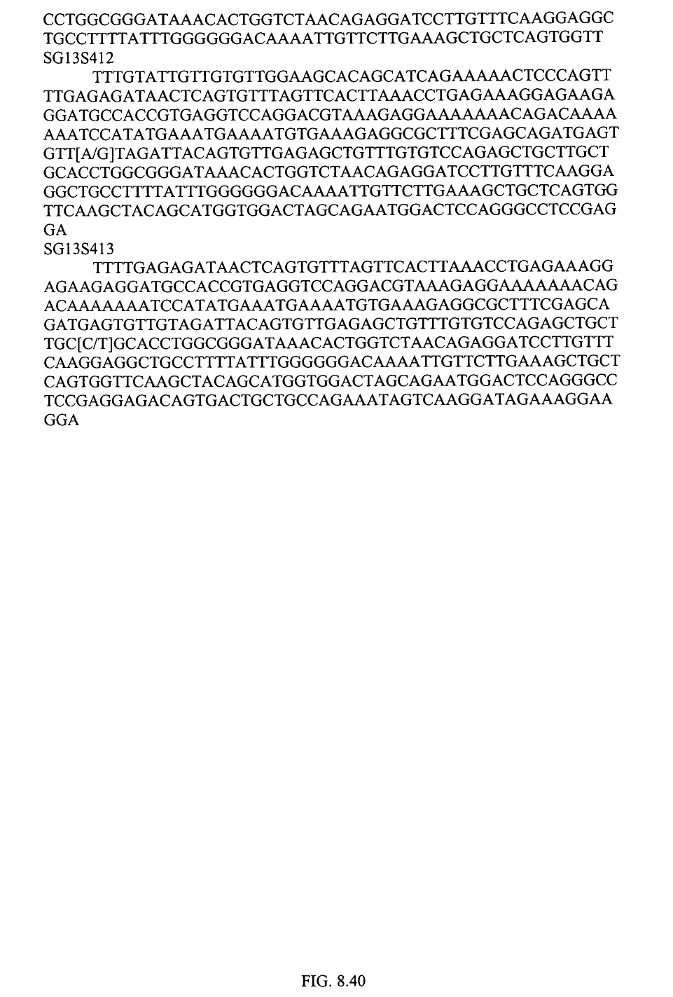

A very large number of microsatellite markers were then added within the central 12 megabase (Mb) segment under the LOD score defined by the drop in one LOD from the peak marker. FIG. 3.1 shows the results from a haplotype association case-control analysis of 437 female MI patients versus 721 controls using combinations of 4 and 5 microsatellite markers to define the test haplotypes. The most significant microsatellite marker haplotype association across this entire 12 Mb segment was found using markers DG13S1103, DG13S166, DG13S1287, DG13S1061 and DG13S301, with alleles 4, 0, 2, 14 and 3, respectively (p-value of 1.02×10⁻⁷). Carrier frequency of this haplotype is 7.3% in female MI patients and 0.3% in controls. There are several other haplotypes that show great association to MI that overlap the first haplotype. The 80 Kb segment that is defined by two markers (DG13S166 and D13S1238) common to all the haplotypes shown in the figure includes only one gene, FLAP (ALOX5AP). A two marker haplotype involving alleles 0 and −2 for markers DG13S166 and D13S1238, respectively, is found in excess in patients. Carrier frequency of this haplotype was estimated to be 27% in female MI patients and 15.4% in controls (p-value 1×10⁻³) . This was our first evidence that variation in the FLAP gene might contribute to MI risk.

To confirm this observation, the FLAP gene was sequenced in its entirety and numerous SNPs were defined. Many of these were used to genotype 437 female MI patients, 1049 male MI patients, and 811 controls. In a case-control study of the MI patients using these data, several haplotypes were found, that were significantly over-represented in the female MI patients compared to controls (see Table 6). These haplotypes were highly correlated to each other. Table 7 shows two haplotypes that are representative of these female MI risk haplotypes. They have relative risks of 2.4 and 4 and are carried by 23% and 13% of female MI patients, respectively. Table 8 shows that these same haplotypes show association to male MI although with lower relative risks.

In an effort to identify haplotypes involving only SNP markers that associate with MI, more SNPs were identified by sequencing the FLAP gene and the region flanking the gene. Currently, a total number of 45 SNPs have been genotyped on 1343 patients and 624 unrelated controls. Two correlated series of SNP haplotypes were observed in excess in patients, denoted as A and B in Table 9. The length of the haplotypes varies between 33 and 69 Kb, and the haplotypes cover one or two blocks of linkage disequilibrium. Both series of haplotypes contain the common allele G of the SNP SG13S25. All haplotypes in the A series contain the SNP DG00AAHID, while all haplotypes in the B series contain the SNP DG00AAHII. In the B series, the haplotypes B4, B5, and B6 have a relative risk (RR) greater than 2 and with allelic frequencies above 10%. The haplotypes in A series have slightly lower RR and lower p-values, but higher frequency (15-16%). The haplotypes in series B and A are strongly correlated, i.e. the haplotypes in B define a subset of the haplotypes in A. Hence, haplotypes B are more specific than A. Haplotypes A are however more sensitive, i.e. they capture more individuals with the putative mutation, as is observed in the population attributable risk which is less for B than for A. Furthermore, these haplotypes show similar risk ratios and allelic frequency for early-onset patients (defined as onset of first MI before the age of 55) and for both gender. In addition, analyzing various groups of patients with known risk factors, such as hypertension, high cholesterol, smoking and diabetes, did not reveal any significant correlation with these haplotypes, indicating that the haplotypes in the FLAP gene represent an independent genetic susceptibility factor for MI.

The FLAP gene encodes for a protein that is required for leukotriene synthesis (LTA4, LTB4, LTC4, LTD4, LTE4). Inhibitors of its function impede translocation of 5-lipoxygenase from the cytoplasm to the cell membrane and inhibit activation of 5-lipoxygenase. The leukotrienes are potent inflammatory lipid mediators derived from arachidonic acid that can potentially contribute to development of atherosclerosis and destabilization of atherosclerotic plaques throu lipid oxidation and/or proinflammatory effects. Allen et al., (Circulation. 97: 2406-2413(1998)) described a novel mechanism in which atherosclerosis is associated with the appearance of a leukotriene receptor(s) capable of inducing hyperreactivity of human epicardial coronary arteries in response to LTC4 and LTD4. Allen et al. show a photomicrograph of a section of human atherosclerotic coronary artery a positive staining of a number of members of the leukotriene pathway, including FLAP. Mehrabian et al. described the identification of 5-Lipoxygenase (5-LO) as a major gene contributing to atherosclerosis susceptibility in mice. Mehrabian et al. described that heterozygous deficiency for the enzyme in a knockout model decreased the atherosclerotic lesion size in LDL−/− mice by about 95%. Mehrabian et al show that the enzyme is expressed abundantly in macrophage-rich regions of atherosclerotic lesions, and suggested that 5-LO and/or its products might act locally to promote lesion development (Mehrabian et al., Circulation Research. 91:120 (2002)). Studies of FLAP inhibition in animal models of atheroscerosis are scarce. However, in a rabbit model of acute MI assesssed 72 hours after coronary artery ligation the FLAP-inhibitor BAYx1005 markedly reduced mortality, from 65% to 25%, and blocked the increase in CPK and neutrophil accumulation as well as the ECG-changes observed in sham treated animals (J. Pharmacol. Exp. Ther., 276:332 (1996)).

Mutations and/or polymorphisms within the FLAP nucleic acid, and other members of the same pathway (e.g., 5-lipoxygenase, LTA4 Hydrolase, LTB4 receptors, LTC4 Synthase, and CysLT2 receptor), that show association with the disease, can be used as a diagnostic test. The members of the 5-LO pathway in particular are valuable therapeutic targets for myocardial infarction.

TABLE 1
The marker map for chromosome 13 used in the linkage
analysis.

| Location (cM) | Marker |
|---|---|
| 6 | D13S175 |
| 9.8 | D13S1243 |
| 13.5 | D13S1304 |
| 17.2 | D13S217 |
| 21.5 | D13S289 |
| 25.1 | D13S171 |
| 28.9 | D13S219 |
| 32.9 | D13S218 |
| 38.3 | D13S263 |
| 42.8 | D13S326 |
| 45.6 | D13S153 |
| 49.4 | D13S1320 |
| 52.6 | D13S1296 |
| 55.9 | D13S156 |
| 59.8 | D13S1306 |
| 63.9 | D13S170 |
| 68.7 | D13S265 |
| 73 | D13S167 |
| 76.3 | D13S1241 |
| 79.5 | D13S1298 |
| 81.6 | D13S1267 |
| 84.7 | D13S1256 |
| 85.1 | D13S158 |
| 87 | D13S274 |
| 93.5 | D13S173 |
| 96.7 | D13S778 |
| 102.7 | D13S1315 |
| 110.6 | D13S285 |
| 115 | D13S293 |

TABLE 2
Marker Map for the second step of Linkage Analysis

| Location (cM) | Marker |
|---|---|
| 1.758 | D13S175 |
| 9.235 | D13S787 |
| 11.565 | D13S1243 |
| 16.898 | D13S221 |
| 17.454 | D13S1304 |
| 18.011 | D13S1254 |
| 18.59 | D13S625 |
| 19.308 | D13S1244 |
| 19.768 | D13S243 |
| 22.234 | D13S1250 |
| 22.642 | D13S1242 |
| 22.879 | D13S217 |
| 25.013 | D13S1299 |
| 28.136 | D13S289 |
| 28.678 | D13S290 |
| 29.134 | D13S1287 |
| 30.073 | D13S260 |
| 31.98 | D13S171 |
| 32.859 | D13S267 |
| 33.069 | D13S1293 |
| 33.07 | D13S620 |
| 34.131 | D13S220 |
| 36.427 | D13S219 |
| 39.458 | D13S1808 |
| 40.441 | D13S218 |
| 41.113 | D13S1288 |
| 41.996 | D13S1253 |
| 42.585 | D13S1248 |
| 44.288 | D13S1233 |
| 44.377 | D13S263 |
| 45.535 | D13S325 |
| 45.536 | D13S1270 |
| 45.537 | D13S1276 |
| 49.149 | D13S326 |
| 49.532 | D13S1272 |
| 52.421 | D13S168 |
| 52.674 | D13S287 |
| 60.536 | D13S1320 |
| 64.272 | D13S1296 |
| 71.287 | D13S156 |
| 76.828 | D13S1306 |
| 77.86 | D13S170 |
| 82.828 | D13S265 |
| 91.199 | D13S1241 |
| 93.863 | D13S1298 |
| 97.735 | D13S779 |
| 100.547 | D13S1256 |
| 102.277 | D13S274 |
| 111.885 | D13S173 |
| 112.198 | D13S796 |
| 115.619 | D13S778 |
| 119.036 | D13S1315 |
| 126.898 | D13S285 |
| 131.962 | D13S293 |

Table 3 shows the exons with position that encode the FLAP protein, markers, polymorphisms and SNPs identified within the genomic sequence by the methods described herein.

| NCBI | NCBI | | | | | |
|---|---|---|---|---|---|---|
| build34; | build34; | | | | | |
| start on | stop on | | | | | |
| chr. 13 | chr. 13 | SNP/marker/ | | | public | |
| (bp) | (bp) | exon name | alias1 | alias2 | SNP | Variation |
| 28932432 | 28932432 | SG13S421 | | DG00AAFQR | rs1556428 | A/G |
| 28960356 | 28960356 | SG13S417 | | SNP13B_R1028729 | rs1028729 | C/T |
| 28965803 | 28965803 | SG13S418 | | SNP13B_Y1323898 | rs1323898 | A/G |
| 28974627 | 28974627 | SG13S44 | | | | A/G |
| 28975101 | 28975101 | SG13S45 | | | | C/G |
| 28975315 | 28975315 | SG13S46 | | | | A/G |
| 28975353 | 28975353 | 3G13S50 | | | | C/T |
| 28975774 | 28975774 | SG13S52 | | | | A/G |
| 28985244 | 28985244 | SG13S53 | | | rs1408167 | A/C |
| 28985303 | 28985303 | SG13S55 | | | rs1408169 | A/G |
| 28985423 | 28985423 | SG13S56 | | | | G/T |
| 28985734 | 28985734 | SG13S57 | | | rs6490471 | C/T |
| 28985902 | 28985902 | SG13S58 | | | rs6490472 | A/G |
| 29003869 | 29003869 | SG13S59 | | | | C/G |
| 29004696 | 29004696 | SG13S60 | | | | A/G |
| 29007670 | 29007670 | SG13S419 | | SNP13B_K912392 | rs912392 | C/T |
| 29015410 | 29015410 | SG13S61 | | | | C/T |
| 29025792 | 29025792 | SG13S62 | | | | C/T |
| 29026202 | 29026202 | SG13S63 | | | rs7997114 | A/G |
| 29026668 | 29026668 | SG13364 | | | | A/G |
| 29038707 | 29038707 | SG13S65 | | | | A/G |
| 29042180 | 29042180 | SG13S420 | | DG00AAFIV | rs2248564 | A/T |
| 29049355 | 29049355 | SG13S66 | | | | A/G |
| 29049446 | 29049446 | SG13S67 | | | | C/T |
| 29050416 | 29050416 | SG13S69 | | | | A/C |
| 29059348 | 29059348 | SG13S70 | | | | A/G |
| 29059383 | 29059383 | SG13S71 | | | | A/G |
| 29059402 | 29059402 | SG13S72 | | | | G/T |
| 29063702 | 29063949 | D13S289 | | | | |
| 29064359 | 29064753 | DG13S166 | | | | |
| 29066272 | 29066272 | SG13S73 | | | | A/G |
| 29070551 | 29070551 | SG13S99 | SNP_13_Y1323892 | DG00AAFIU | rs1323892 | C/T |
| 29081983 | 29081983 | SG13S382 | FLA267479 | | | A/G |
| 29082200 | 29082200 | SG13S383 | FLA267696 | | | A/G |
| 29082357 | 29082357 | SG13S384 | FLA267853 | | | A/G |
| 29083350 | 29083350 | SG13S381 | FLA268846 | DG00AAJER | | C/G |
| 29083518 | 29083518 | SG13S366 | FLA269014 | DG00AAJES | rs4312166 | A/G |
| 29085102 | 29085102 | SG13S385 | FLA270742 | | | C/T |
| 29085190 | 29085190 | SG13S386 | FLA270830 | | | A/G |
| 29086224 | 29086224 | SG13S1 | FLA271864 | | | G/T |
| 29087473 | 29087473 | SG13S2 | FLA273371 | | | A/G |
| 29088090 | 29088090 | SG13S367 | FLA273988 | DG00AAJEU | rs4474551 | A/G |
| 29088186 | 29088186 | SG13S388 | FLA274084 | | | A/G |
| 29088473 | 29088473 | SG13S10 | FLA274371 | | | A/T |
| 29089044 | 29089044 | SG13S3 | FLA274942 | | | C/T |
| 29089886 | 29089886 | SG13S368 | FLA275784 | DG00AAJEV | | C/T |
| 29090025 | 29090025 | SG13S369 | FLA275923 | DG00AAJEW | | G/T |
| 29090054 | 29090054 | SG13S370 | FLA275952 | DG00AAJEX | | A/G |
| 29090997 | 29090997 | SG13S4 | FLA276895 | | | G/C |
| 29091307 | 29091307 | SG13S5 | FLA277205 | | rs4238133 | G/T |
| 29091580 | 29091580 | SC13S389 | FLA277478 | | | A/G |
| 29091780 | 29091780 | SG13S90 | FLA277678 | | | A/C |
| 29092287 | 29092287 | SG13S390 | FLA278185 | | rs5004913 | A/G |
| 29092536 | 29092536 | SG13S6 | FLA278434 | | | A/G |
| 29092594 | 29092594 | SG13S391 | FLA278492 | | | A/G |
| 29092947 | 29092947 | SG13S392 | FLA278845 | | | G/T |
| 29093964 | 29093964 | SG13S371 | FLA279888 | DG00AAJEY | rs4409939 | A/G |
| 29094259 | 29094259 | SG13S372 | FLA280183 | DG00AAJEZ | | A/G |
| 29094999 | 29094999 | SG13S393 | FLA280923 | | | A/T |
| 29096688 | 29096688 | SG13S373 | FLA282612 | DG00AAJFA | | A/G |
| 29096813 | 29096813 | SG13S374 | FLA282737 | DG00AAJFB | | A/G |
| 29096874 | 29096874 | SG13S375 | FLA282798 | DG00AAJFC | | C/T |
| 29096962 | 29096962 | SG13S376 | FLA282886 | DG00AAJFD | | A/G |
| 29097476 | 29097476 | SG13S394 | FLA283400 | | | C/G |
| 29097553 | 29097553 | SG13S25 | FLA283477 | | | A/G |
| 29098486 | 29098486 | SG13S395 | FLA284410 | | | A/G |
| 29098891 | 29098891 | SG13S396 | FLA284815 | | | A/C |
| 29098979 | 29098979 | SG13S397 | FLA284903 | | | C/T |
| 29101965 | 29101965 | SG13S377 | FLA287889 | DG00AAJFF | | A/G |
| 29103909 | 29103909 | SG13S189 | FLA289833 | | | C/G |
| 29104271 | 29104271 | SG13S100 | FLA290195 | DG00AAHIK | rs4073259 | A/G |
| 29104629 | 29104629 | SG13S398 | FLA290553 | | | C/G |
| 29104646 | 29104646 | SG13S94 | FLA290570 | | rs4073261 | C/T |
| 29105099 | 29105099 | SG13S101 | FLA291023 | | rs4075474 | C/T |
| 29106329 | 29106329 | SG13S95 | FLA292253 | | | G/T |
| 29106652 | 29106652 | SG13S102 | FLA292576 | | | A/T |
| 29107138 | 29107138 | SG13S103 | FLA293062 | | | C/T |
| 29107404 | 29107404 | SG13S104 | FLA293328 | | | A/G |
| 29107668 | 29107812 | EXON1 | | | | |
| 29107830 | 29107830 | SG13S191 | FLA293754 | DG00AAFJT | rs4769055 | A/C |
| 29108398 | 29108398 | SG13S105 | FLA294322 | | | A/G |
| 29108579 | 29108579 | SG13S106 | FLA294503 | DG00AAHII | | A/G |
| 29108919 | 29108919 | SG13S107 | FLA294843 | | rs4075131 | A/G |
| 29108972 | 29108972 | SG13S108 | FLA294896 | | rs4075132 | C/T |
| 29109112 | 29109112 | SG13S109 | FLA295036 | | | A/G |
| 29109182 | 29109182 | SG13S110 | FLA295106 | | | A/G |
| 29109344 | 29109344 | SG13S111 | FLA295268 | | rs4597169 | C/T |
| 29109557 | 29109557 | SG13S112 | FLA295481 | | | C/T |
| 29109773 | 29109773 | SG13S113 | FLA295697 | | rs4293222 | C/G |
| 29110096 | 29110096 | SG13S114 | FLA296020 | DG00AAHID | | A/T |
| 29110178 | 29110178 | SG13S115 | FLA296102 | | | A/T |
| 29110508 | 29110508 | SG13S116 | FLA296432 | | rs4769871 | C/T |
| 29110630 | 29110630 | SG13S117 | FLA296554 | | rs4769872 | A/G |
| 29110689 | 29110689 | SG13S118 | FLA296613 | | rs4769873 | C/T |
| 29110862 | 29110862 | SG13S119 | FLA296786 | | | A/G |
| 29111889 | 29111889 | SG13S120 | FLA297813 | | | C/T |
| 29112174 | 29112174 | SG13S121 | FLA298098 | DG00AAHIJ | rs4503649 | A/G |
| 29112264 | 29112264 | SG13S122 | FLA298188 | DG00AAHIH | | A/G |
| 29112306 | 29112306 | SG13S123 | FLA298230 | | | C/T |
| 29112455 | 29112455 | SG13S43 | FLA298379 | | rs3885907 | A/C |
| 29112583 | 29112583 | SG13S399 | FLA298507 | | | A/C |
| 29112680 | 29112680 | SG13S124 | FLA298604 | | rs3922435 | C/T |
| 29113139 | 29113139 | SG13S125 | FLA299063 | | | A/G |
| 29114056 | 29114056 | SG13S400 | FLA299980 | | | A/G |
| 29114738 | 29114738 | SG13S126 | FLA300662 | | | A/G |
| 29114940 | 29114940 | SG13S127 | FLA300864 | | | A/G |
| 29115878 | 29115878 | SG13S128 | FLA302094 | | rs4254165 | A/G |
| 29116020 | 29116020 | SG13S129 | FLA302236 | | rs4360791 | A/G |
| 29116068 | 29116068 | SG13S130 | FLA302284 | | | G/T |
| 29116196 | 29116296 | EXON2 | | | | |
| 29116249 | 29116249 | SG13S190 | FLA302465 | | | C/T |
| 29116308 | 29116308 | SG13S192 | FLA302524 | B_SNP_302524 | rs3803277 | A/C |
| 29116344 | 29116344 | SG13S193 | FLA302560 | | | A/G |
| 29116401 | 29116401 | SG13S88 | FLA302617 | B_SNP_302617 | rs3803278 | C/T |
| 29116688 | 29116688 | SG13S131 | FLA302904 | | | C/T |
| 29117133 | 29117133 | SG13S132 | FLA303349 | | | A/C |
| 29117546 | 29117546 | SG13S133 | FLA303762 | | rs4356336 | C/T |
| 29117553 | 29117553 | SG13S38 | FLA303769 | | rs4584668 | A/T |
| 29117580 | 29117580 | SG13S134 | FLA303796 | | | C/T |
| 29117741 | 29117741 | SG13S135 | FLA303957 | | rs4238137 | C/T |
| 29117954 | 29117954 | SG13S136 | FLA304170 | | rs4147063 | C/T |
| 29118118 | 29118118 | SG13S137 | FLA304334 | DG00AAHIG | rs4147064 | C/T |
| 29118815 | 29118815 | SG13S86 | FLA305031 | | | A/G |
| 29118873 | 29118873 | SG13S87 | FLA305089 | DG00AAHOJ | | A/G |
| 29119069 | 29119069 | SG13S138 | FLA305285 | | | C/T |
| 29119138 | 29119138 | SG13S139 | FLA305354 | | | C/G |
| 29119289 | 29119289 | SG13S140 | FLA305505 | | | A/G/T |
| 29119462 | 29119462 | SG13S141 | FLA305678 | | | C/T |
| 29119740 | 29119740 | SG13S39 | FLA305956 | | | G/T |
| 29120939 | 29120939 | SG13S142 | FLA307155 | | rs4387455 | C/T |
| 29120949 | 29120949 | SG13S143 | FLA307165 | | rs4254166 | C/T |
| 29121342 | 29121342 | SG13S144 | FLA307558 | | rs4075692 | A/G |
| 29121572 | 29121572 | SG13S145 | FLA307788 | | | C/G |
| 29121988 | 29121988 | SG13S146 | FLA308204 | | | C/T |
| 29122253 | 29122253 | SG13S26 | FLA308469 | | | C/T |
| 29122283 | 29122283 | SG13S27 | FLA308499 | | | A/G |
| 29122294 | 29122294 | SG13S147 | FLA308510 | | | C/T |
| 29122298 | 29122298 | SG13S28 | FLA308514 | | | G/T |
| 29122311 | 29122311 | SG13S148 | FLA308527 | | | G/T |
| 29123370 | 29123370 | SG13S98 | FLA309586 | | | G/T |
| 29123635 | 29123635 | SG13S149 | FLA309851 | | | A/G |
| 29123643 | 29123643 | SG13S29 | FLA309859 | | | A/C |
| 29124188 | 29124259 | EXON3 | | | | |
| 29124441 | 29124441 | SG13S89 | FLA310657 | B_SNP_310657 | rs4769874 | A/G |
| 29124906 | 29124906 | SG13S96 | FLA311122 | | rs4072653 | A/G |
| 29125032 | 29125032 | SG13S150 | FLA311248 | | | C/G |
| 29125521 | 29125521 | SG13S401 | FLA311737 | | | C/T |
| 29125822 | 29125822 | SG13S151 | FLA312038 | | | C/T |
| 29125840 | 29125840 | SG13S30 | FLA312056 | | | G/T |
| 29127301 | 29127301 | SG13S31 | FLA313550 | | | C/T |
| 29128080 | 29128162 | EXON4 | | | | |
| 29128284 | 29128284 | SG13S152 | FLA314500 | | | C/G |
| 29128316 | 29128316 | SG13S402 | FLA314532 | | rs4468448 | C/T |
| 29128798 | 29128798 | SG13S403 | FLA315014 | | rs4399410 | A/G |
| 29129016 | 29129016 | SG13S153 | FLA315232 | | | A/T |
| 29129139 | 29129139 | SG13S97 | FLA315355 | | | A/G |
| 29129154 | 29129154 | SG13S154 | FLA315370 | | | C/T |
| 29129395 | 29129395 | SG13S40 | FLA315611 | | | G/T |
| 29129915 | 29129915 | SG13S155 | FLA316131 | | rs4769875 | A/G |
| 29130192 | 29130192 | SG13S156 | FLA316408 | | | A/C |
| 29130256 | 29130256 | SG13S157 | FLA316472 | | | A/G |
| 29130299 | 29130299 | SG13S158 | FLA316515 | | | A/C |
| 29130353 | 29130353 | SG13S159 | FLA316569 | | | G/T |
| 29130391 | 29130391 | SG13S160 | FLA316607 | | | C/T |
| 29130547 | 29130547 | SG13S32 | FLA316763 | | | A/C |
| 29131280 | 29131280 | SG13S161 | FLA317496 | | | A/G |
| 29131403 | 29131403 | SG13S162 | FLA317619 | | | A/G |
| 29131404 | 29131404 | SG13S163 | FLA317620 | | | C/T |
| 29131431 | 29131431 | SG13S164 | FLA317647 | | rs4769058 | C/T |
| 29131517 | 29131517 | SG13S165 | FLA317733 | | | A/T |
| 29131528 | 29131528 | SG13S166 | FLA317744 | | rs4769059 | C/T |
| 29131599 | 29131599 | SG13S167 | FLA317815 | | rs4769876 | A/G |
| 29132003 | 29132003 | SG13S168 | FLA318219 | | | A/C |
| 29133753 | 29133753 | SG13S33 | FLA319969 | | | G/T |
| 29134045 | 29134045 | SG13S41 | FLA320261 | | | A/G |
| 29134177 | 29134177 | SG13S169 | FLA320393 | | | A/G |
| 29134379 | 29134379 | SG13S404 | FLA320595 | | rs4427651 | G/T |
| 29135558 | 29135558 | SG13S170 | FLA321774 | | rs3935645 | C/T |
| 29135640 | 29135640 | SG13S171 | FLA321856 | | rs3935644 | A/G |
| 29135750 | 29135750 | SG13S172 | FLA321966 | | | A/G |
| 29135809 | 29135809 | SG13S173 | FLA322025 | | | A/T |
| 29135877 | 29135877 | SG13S42 | FLA322093 | | rs4769060 | A/G |
| 29136080 | 29136556 | EXON5 | | | | |
| 29136290 | 29136290 | SG13S194 | FLA322506 | | | C/T |
| 29136462 | 29136462 | SG13S195 | FLA322678 | | rs1132340 | A/G |
| 29136797 | 29136797 | SG13S174 | FLA323013 | | | A/G |
| 29137100 | 29137100 | SG13S34 | FLA323316 | | | G/T |
| 29137150 | 29137150 | SG13S175 | FLA323366 | | | A/G |
| 29137607 | 29137607 | SG13S176 | FLA323823 | | | A/G |
| 29137651 | 29137651 | SG13S177 | FLA323867 | | | C/T |
| 29137905 | 29137905 | SG13S178 | FLA324121 | | | C/G |
| 29138117 | 29138117 | SG13S35 | FLA324333 | | | A/G |
| 29138375 | 29138375 | SG13S179 | FLA324591 | | | A/G |
| 29138385 | 29138385 | SG13S180 | FLA324601 | | | C/T |
| 29138633 | 29138633 | SG13S181 | FLA324849 | DG00AAHIF | rs4420371 | C/G |
| 29139153 | 29139153 | SG13S182 | FLA325369 | | | C/T |
| 29139277 | 29139277 | SG13S183 | FLA325493 | | rs4466940 | C/T |
| 29139435 | 29139435 | SG13S184 | FLA325651 | DG00AAHOI | rs4445746 | A/G |
| 29139971 | 29139971 | SG13S185 | FLA326187 | | | A/G |
| 29140441 | 29140441 | SG13S405 | FLA326657 | | | A/G |
| 29140649 | 29140649 | SG13S91 | FLA326865 | | | A/G |
| 29140695 | 29140695 | SG13S186 | FLA326911 | | rs4769877 | A/T |
| 29140703 | 29140703 | SG13S187 | FLA326919 | | | A/G |
| 29140805 | 29140805 | SG13S188 | FLA327021 | DG00AAJFE | | A/G |
| 29141049 | 29141049 | SG13S406 | FLA327265 | | | C/T |
| 29142392 | 29142392 | SG13S92 | FLA328644 | | rs4429158 | C/T |
| 29142397 | 29142397 | SG13S93 | FLA328649 | | | A/G |
| 29142712 | 29142712 | SG13S36 | FLA328964 | | | C/T |
| 29144013 | 29144013 | SG13S407 | FLA330265 | | | C/T |
| 29144203 | 29144203 | SG13S408 | FLA330455 | | | C/T |
| 29144234 | 29144589 | D13S1238 | | | | |
| 29144255 | 29144255 | SG13S7 | FLA330507 | | | C/T |
| 29144877 | 29144877 | SG13S37 | FLA331129 | | | A/G |
| 29144982 | 29144982 | SG13S409 | FLA331234 | | | A/G |
| 29144983 | 29144983 | SG13S8 | FLA331235 | | rs4491352 | A/C |
| 29145122 | 29145122 | SG13S410 | FLA331374 | | rs4319601 | C/T |
| 29145143 | 29145143 | SG13S411 | FLA331395 | | | A/G |
| 29145171 | 29145171 | SG13S9 | FLA331423 | | | C/T |
| 29145221 | 29145221 | SG13S412 | FLA331473 | | rs4769062 | A/G |
| 29145265 | 29145265 | SG13S413 | FLA331517 | | rs4238138 | C/T |

| | minor | start | end |
|---|---|---|---|
| | allele | position in | position in |
| minor | frequency | sequence | sequence |
| allele | (%) | xx | xx |
| G | 10.32 | 432 | 432 |
| G | 30.46 | 28356 | 28356 |
| T | 37.38 | 33803 | 33803 |
| G | 0.545 | 42627 | 42627 |
| G | 1.111 | 43101 | 43101 |
| G | 0.328 | 43315 | 43315 |
| C | 0.495 | 43353 | 43353 |
| A | 6.993 | 43774 | 43774 |
| C | 30.876 | 53244 | 53244 |
| G | 6.731 | 53303 | 53303 |
| T | 0.353 | 53423 | 53423 |
| C | 31.356 | 53734 | 53734 |
| A | 30.935 | 53902 | 53902 |
| G | 5.492 | 71869 | 71869 |
| A | 1.812 | 72696 | 72696 |
| G | 35.00 | 75670 | 75670 |
| C | 1.314 | 83410 | 83410 |
| T | 3.521 | 93792 | 93792 |
| A | 30.031 | 94202 | 94202 |
| A | 1.724 | 94668 | 94668 |
| A | 0.369 | 106707 | 106707 |
| A | 13.66 | 110180 | 110180 |
| A | 20.779 | 117355 | 117355 |
| T | 5.965 | 117446 | 117446 |
| A | 16.923 | 118416 | 118416 |
| A | 34.364 | 127348 | 127348 |
| A | 8.537 | 127383 | 127383 |
| T | 25.536 | 127402 | 127402 |
| | | 131702 | 131949 |
| | | 132359 | 132753 |
| A | 37.302 | 134272 | 134272 |
| C | 6.25 | 138551 | 138551 |
| A | 0.49 | 149983 | 149983 |
| A | 14.08 | 150200 | 150200 |
| G | 0.62 | 150357 | 150357 |
| G | 14.01 | 151350 | 151350 |
| T | 0.58 | 151518 | 151518 |
| C | 30.21 | 153102 | 153102 |
| A | 10.95 | 153190 | 153190 |
| G | 30.00 | 154224 | 154224 |
| A | 27.95 | 155473 | 155473 |
| G | 2.41 | 156090 | 156090 |
| A | 0.39 | 156186 | 156186 |
| T | 10.23 | 156473 | 156473 |
| T | 15.17 | 157044 | 157044 |
| T | 13.60 | 157886 | 157886 |
| G | 12.44 | 158025 | 158025 |
| A | 13.45 | 158054 | 158054 |
| G | 14.59 | 158997 | 158997 |
| T | 26.84 | 159307 | 159307 |
| A | 12.73 | 159580 | 159580 |
| C | 43.67 | 159780 | 159780 |
| A | 12.18 | 160287 | 160287 |
| A | 8.38 | 160536 | 160536 |
| G | 0.62 | 160594 | 160594 |
| T | 12.34 | 160947 | 160947 |
| G | 25.34 | 161964 | 161964 |
| C | 0.24 | 162259 | 162259 |
| T | 25.66 | 162999 | 162999 |
| A | 14.84 | 164688 | 164688 |
| G | 12.37 | 164813 | 164813 |
| C | 14.55 | 164874 | 164874 |
| G | 11.99 | 164962 | 164962 |
| C | 14.66 | 165476 | 165476 |
| A | 12.21 | 165553 | 165553 |
| A | 0.79 | 166486 | 166486 |
| C | 10.15 | 166891 | 166891 |
| C | 3.53 | 166979 | 166979 |
| A | 12.45 | 169965 | 169965 |
| C | 0.62 | 171909 | 171909 |
| G | 31.55 | 172271 | 172271 |
| G | 4.94 | 172629 | 172629 |
| C | 15.51 | 172646 | 172646 |
| T | 27.91 | 173099 | 173099 |
| G | 14.74 | 174329 | 174329 |
| T | 1.17 | 174652 | 174652 |
| T | 1.28 | 175138 | 175138 |
| A | 2.17 | 175404 | 175404 |
| | | 175668 | 175812 |
| A | 30.11 | 175830 | 175830 |
| G | 0.66 | 176398 | 176398 |
| A | 28.31 | 176579 | 176579 |
| G | 14.85 | 176919 | 176919 |
| C | 1.21 | 176972 | 176972 |
| A | 1.04 | 177112 | 177112 |
| G | 0.88 | 177182 | 177182 |
| C | 1.14 | 177344 | 177344 |
| T | 7.10 | 177557 | 177557 |
| C | 22.52 | 177773 | 177773 |
| A | 20.86 | 178096 | 178096 |
| T | 13.83 | 178178 | 178178 |
| T | 4.05 | 178508 | 178508 |
| A | 4.07 | 178630 | 178630 |
| T | 4.07 | 178689 | 178689 |
| A | 1.06 | 178862 | 178862 |
| C | 16.00 | 179889 | 179889 |
| G | 49.36 | 180174 | 180174 |
| A | 29.75 | 180264 | 180264 |
| T | 5.06 | 180306 | 180306 |
| C | 46.23 | 180455 | 180455 |
| C | 1.59 | 180583 | 180583 |
| T | 1.45 | 180680 | 180680 |
| G | 11.32 | 181139 | 181139 |
| A | 3.25 | 182056 | 182056 |
| A | 34.12 | 182738 | 182738 |
| G | 29.63 | 182940 | 182940 |
| A | 45.68 | 183878 | 183878 |
| G | 36.65 | 184020 | 184020 |
| G | 8.07 | 184068 | 184068 |
| | | 184196 | 184296 |
| T | 1.02 | 184249 | 184249 |
| A | 49.57 | 184308 | 184308 |
| A | 0.58 | 184344 | 184344 |
| C | 24.71 | 184401 | 184401 |
| T | 7.19 | 184688 | 184688 |
| A | 1.10 | 185133 | 185133 |
| T | 37.65 | 185546 | 185546 |
| A | 45.50 | 185553 | 185553 |
| T | 1.22 | 185580 | 185580 |
| T | 0.89 | 185741 | 185741 |
| T | 36.69 | 185954 | 185954 |
| T | 29.11 | 186118 | 186118 |
| A | 30.19 | 186815 | 186815 |
| G | 3.29 | 186873 | 186873 |
| T | 36.96 | 187069 | 187069 |
| G | 36.63 | 187138 | 187138 |
| T | 37.34 | 187289 | 187289 |
| C | 1.15 | 187462 | 187462 |
| T | 9.91 | 187740 | 187740 |
| C | 3.36 | 188939 | 188939 |
| T | 36.24 | 188949 | 188949 |
| A | 31.58 | 189342 | 189342 |
| G | 0.45 | 189572 | 189572 |
| T | 1.14 | 189988 | 189988 |
| T | 46.57 | 190253 | 190253 |
| A | 10.34 | 190283 | 190283 |
| T | 8.00 | 190294 | 190294 |
| T | 33.71 | 190298 | 190298 |
| T | 2.29 | 190311 | 190311 |
| G | 1.19 | 191370 | 191370 |
| A | 1.01 | 191635 | 191635 |
| A | 47.88 | 191643 | 191643 |
| | | 192188 | 192259 |
| A | 4.68 | 192441 | 192441 |
| G | 29.72 | 192906 | 192906 |
| C | 8.22 | 193032 | 193032 |
| C | 21.10 | 193521 | 193521 |
| T | 8.57 | 193822 | 193822 |
| T | 23.23 | 193840 | 193840 |
| T | 24.20 | 195301 | 195301 |
| | | 196080 | 196162 |
| C | 23.89 | 196284 | 196284 |
| T | 19.33 | 196316 | 196316 |
| G | 11.50 | 196798 | 196798 |
| T | 3.08 | 197016 | 197016 |
| A | 9.72 | 197139 | 197139 |
| T | 0.98 | 197154 | 197154 |
| T | 2.24 | 197395 | 197395 |
| A | 1.43 | 197915 | 197915 |
| A | 1.80 | 198192 | 198192 |
| G | 2.38 | 198256 | 198256 |
| A | 0.61 | 198299 | 198299 |
| G | 2.55 | 198353 | 198353 |
| T | 0.83 | 198391 | 198391 |
| C | 48.50 | 198547 | 198547 |
| G | 2.44 | 199280 | 199280 |
| G | 2.45 | 199403 | 199403 |
| C | 2.45 | 199404 | 199404 |
| C | 2.55 | 199431 | 199431 |
| T | 20.00 | 199517 | 199517 |
| T | 2.46 | 199528 | 199528 |
| A | 3.50 | 199599 | 199599 |
| C | 8.39 | 200003 | 200003 |
| T | 8.99 | 201753 | 201753 |
| G | 5.41 | 202045 | 202045 |
| G | 4.12 | 202177 | 202177 |
| G | 38.33 | 202379 | 202379 |
| C | 32.77 | 203558 | 203558 |
| G | 48.03 | 203640 | 203640 |
| G | 1.67 | 203750 | 203750 |
| A | 0.68 | 203809 | 203809 |
| G | 42.44 | 203877 | 203877 |
| | | 204080 | 204556 |
| T | 0.30 | 204290 | 204290 |
| G | 2.46 | 204462 | 204462 |
| G | 0.56 | 204797 | 204797 |
| G | 30.23 | 205100 | 205100 |
| A | 2.40 | 205150 | 205150 |
| A | 2.24 | 205607 | 205607 |
| T | 1.64 | 205651 | 205651 |
| C | 1.40 | 205905 | 205905 |
| A | 9.52 | 206117 | 206117 |
| A | 48.14 | 206375 | 206375 |
| T | 2.50 | 206385 | 206385 |
| C | 49.41 | 206633 | 206633 |
| T | 2.36 | 207153 | 207153 |
| T | 12.07 | 207277 | 207277 |
| A | 16.67 | 207435 | 207435 |
| G | 7.66 | 207971 | 207971 |
| A | 9.66 | 208441 | 208441 |
| A | 7.78 | 208649 | 208649 |
| A | 25.71 | 208695 | 208695 |
| A | 1.43 | 208703 | 208703 |
| G | 4.71 | 208805 | 208805 |
| T | 0.56 | 209049 | 209049 |
| T | 8.33 | 210392 | 210392 |
| A | 7.23 | 210397 | 210397 |
| C | 15.88 | 210712 | 210712 |
| T | 3.29 | 212013 | 212013 |
| T | 0.30 | 212203 | 212203 |
| | | 212234 | 212589 |
| T | 16.28 | 212255 | 212255 |
| G | 16.70 | 212877 | 212877 |
| A | 1.93 | 212982 | 212982 |
| C | 30.64 | 212983 | 212983 |
| T | 20.57 | 213122 | 213122 |
| A | 1.54 | 213143 | 213143 |
| C | 16.37 | 213171 | 213171 |
| A | 7.42 | 213221 | 213221 |
| T | 1.91 | 213265 | 213265 |

TABLE 4
Significant 4 microsatellite marker haplotypes.

| 4 | | | | | | | | | | | | | |
|---|---|---|---|---|---|---|---|---|---|---|---|---|---|
| markers | : | pos.rr-frqgt1perc | | | | | | | | | | | |
| Length | p-val | RR | N_af | P_al | P_ca | N_ct | P_al | P_ca | Alleles | | | | Markers |
| 0.88 | 4.71E−06 | 6.23 | 428 | 0.065 | 0.125 | 721 | 0.011 | 0.022 | 0 | −12 | −6 | 0 | DG13S80 |
| | | | | | | | | | | | | | DG13S83 |
| | | | | | | | | | | | | | DG13S1110 |
| | | | | | | | | | | | | | DG13S163 |
| 0.82 | 8.60E−06 | INF | 438 | 0.032 | 0.062 | 720 | 0 | 0 | 0 | 4 | 2 | 14 | DG13S111 |
| | | | | | | | | | | | | | 1 DG13S1103 |
| | | | | | | | | | | | | | D13S1287 |
| | | | | | | | | | | | | | DG13S1061 |
| 0.67 | 6.98E−06 | 19.91 | 435 | 0.03 | 0.059 | 721 | 0.002 | 0.003 | 8 | 6 | 0 | 8 | DG13S1103 |
| | | | | | | | | | | | | | DG13S163 |
| | | | | | | | | | | | | | D13S290 |
| | | | | | | | | | | | | | DG13S1061 |
| 0.767 | 4.85E−06 | 26.72 | 436 | 0.048 | 0.094 | 721 | 0.002 | 0.004 | 0 | 0 | 2 | 12 | DG13S1101 |
| | | | | | | | | | | | | | DG13S166 |
| | | | | | | | | | | | | | D13S1287 |
| | | | | | | | | | | | | | DG13S1061 |
| 0.515 | 1.93E−06 | INF | 422 | 0.048 | 0.094 | 721 | 0 | 0 | 2 | 0 | 0 | 6 | DG13S166 |
| | | | | | | | | | | | | | DG13S163 |
| | | | | | | | | | | | | | D13S290 |
| | | | | | | | | | | | | | DG13S1061 |
| 0.864 | 1.68E−06 | INF | 424 | 0.024 | 0.048 | 717 | 0 | 0 | 0 | 2 | 0 | −16 | DG13S166 |
| | | | | | | | | | | | | | DG13S163 |
| | | | | | | | | | | | | | DG13S1061 |
| | | | | | | | | | | | | | DG13S293 |
| 0.927 | 5.38E−06 | INF | 435 | 0.034 | 0.067 | 720 | 0 | 0 | 4 | 2 | 14 | 3 | DG13S1103 |
| | | | | | | | | | | | | | D13S1287 |
| | | | | | | | | | | | | | DG13S1061 |
| | | | | | | | | | | | | | DG13S301 |

Length = length of haplotype in Mb.
P-val = p-value.
RR = Relative risk.
N af = Number of patients.
P al = allelic frequency of haplotype.
P ca = carrier frequency of haplotype.
N ct = number of controls.
Alleles = alleles in the haplotype.
Markers = markers in the haplotype.

Alleles #'s: For SNP alleles A=0, C=1, G=2, T=3; for microsatellite alleles: the CEPH sample (Centre d'Etudes du Polymorphisme Humain, genomics repository) is used as a reference, the lower allele of each microsatellite in this sample is set at 0 and all other alleles in other samples are numbered according in relation to this reference. Thus allele1 is 1 bp longer than the lower allele in the CEPH sample, allele 2 is 2 bp longer than the lower allele in the CEPH sample, allele 3 is 3 bp longer than the lower allele in the CEPH sample, allele 4 is 4 bp longer than the lower allele in the CEPH sample, allele −1 is 1 bp shorter than the lower allele in the CEPH sample, allele −2 is 2 bp shorter than the lower allele in the CEPH sample, and so on.

TABLE 5
Significamt 5 microsatellite marker haplotypes.

| 5markers | : | pos.rr-frqgt1perc | | | | | | | | | | | | |
|---|---|---|---|---|---|---|---|---|---|---|---|---|---|---|
| Length | p-val | RR | N_af | P_al | P_ca | N_ct | P_al | P_ca | Alleles | | | | | Markers |
| 0.851 | 7.45E−06 | 15.43 | 413 | 0.034 | 0.067 | 715 | 0.002 | 0.005 | 0 | 18 | 0 | 0 | 0 | DG13S79 |
| | | | | | | | | | | | | | | D13S1299 |
| | | | | | | | | | | | | | | DG13S87 |
| | | | | | | | | | | | | | | D13S1246 |
| | | | | | | | | | | | | | | DG13S166 |
| 0.964 | 8.07E−06 | INF | 437 | 0.023 | 0.045 | 721 | 0 | 0 | 0 | −12 | 6 | 8 | 6 | DG13S79 |
| | | | | | | | | | | | | | | DG13S83 |
| | | | | | | | | | | | | | | DG13S1104 |
| | | | | | | | | | | | | | | DG13S1103 |
| | | | | | | | | | | | | | | DG13S163 |
| 0.964 | 2.38E−06 | INF | 437 | 0.026 | 0.052 | 720 | 0 | 0 | 0 | 6 | 0 | 8 | 6 | DG13S79 |
| | | | | | | | | | | | | | | DG13S1104 |
| | | | | | | | | | | | | | | DG13S172 |
| | | | | | | | | | | | | | | DG13S1103 |
| | | | | | | | | | | | | | | DG13S163 |
| 0.931 | 7.05E−06 | 5.8 | 429 | 0.068 | 0.131 | 721 | 0.012 | 0.025 | 0 | −6 | 0 | 0 | −2 | DG13S79 |
| | | | | | | | | | | | | | | DG13S1110 |
| | | | | | | | | | | | | | | DG13S175 |
| | | | | | | | | | | | | | | DG13S166 |
| | | | | | | | | | | | | | | D13S1238 |
| 0.964 | 8.13E−06 | INF | 434 | 0.021 | 0.041 | 721 | 0 | 0 | 0 | 3 | 8 | 2 | 6 | DG13S79 |
| | | | | | | | | | | | | | | DG13S1098 |
| | | | | | | | | | | | | | | DG13S1103 |
| | | | | | | | | | | | | | | DG13S166 |
| | | | | | | | | | | | | | | DG13S163 |
| 0.597 | 9.78E−06 | 4.58 | 428 | 0.074 | 0.143 | 717 | 0.017 | 0.034 | −6 | 0 | 0 | 0 | −2 | DG13S1110 |
| | | | | | | | | | | | | | | DG13S89 |
| | | | | | | | | | | | | | | DG13S175 |
| | | | | | | | | | | | | | | DG13S166 |
| | | | | | | | | | | | | | | D13S1238 |
| 0.896 | 6.92E−06 | INF | 428 | 0.026 | 0.051 | 721 | 0 | 0 | −12 | −6 | 0 | −2 | 2 | DG13S83 |
| | | | | | | | | | | | | | | DG13S1110 |
| | | | | | | | | | | | | | | DG13S166 |
| | | | | | | | | | | | | | | D13S1238 |
| | | | | | | | | | | | | | | D13S290 |
| 0.722 | 2.18E−06 | INF | 453 | 0.026 | 0.051 | 738 | 0 | 0 | −6 | 0 | 0 | −2 | 2 | DG13S1110 |
| | | | | | | | | | | | | | | D13S289 |
| | | | | | | | | | | | | | | DG13S166 |
| | | | | | | | | | | | | | | D13S1238 |
| | | | | | | | | | | | | | | D13S290 |
| 0.982 | 7.88E−06 | INF | 437 | 0.028 | 0.055 | 721 | 0 | 0 | 0 | 0 | 4 | 2 | 14 | DG13S87 |
| | | | | | | | | | | | | | | DG13S175 |
| | | | | | | | | | | | | | | DG13S1103 |
| | | | | | | | | | | | | | | D13S1287 |
| | | | | | | | | | | | | | | DG13S1061 |
| 0.841 | 8.88E−06 | INF | 438 | 0.032 | 0.062 | 720 | 0 | 0 | 0 | 0 | 4 | 2 | 14 | DG13S89 |
| | | | | | | | | | | | | | | DG13S1111 |
| | | | | | | | | | | | | | | DG13S1103 |
| | | | | | | | | | | | | | | D13S1287 |
| | | | | | | | | | | | | | | DG13S1061 |
| 0.841 | 9.67E−07 | INF | 435 | 0.029 | 0.057 | 721 | 0 | 0 | 0 | 8 | 6 | 0 | 8 | DG13S89 |
| | | | | | | | | | | | | | | DG13S1103 |
| | | | | | | | | | | | | | | DG13S163 |
| | | | | | | | | | | | | | | D13S290 |
| | | | | | | | | | | | | | | DG13S1061 |
| 0.982 | 7.90E−06 | 18.63 | 437 | 0.026 | 0.052 | 721 | 0.001 | 0.003 | 0 | 4 | 0 | 2 | 14 | DG13S87 |
| | | | | | | | | | | | | | | DG13S1103 |
| | | | | | | | | | | | | | | DG13S166 |
| | | | | | | | | | | | | | | D13S1287 |
| | | | | | | | | | | | | | | DG13S1061 |
| 0.841 | 3.52E−06 | 28.52 | 436 | 0.048 | 0.094 | 721 | 0.002 | 0.004 | 0 | 0 | 0 | 2 | 12 | DG13S89 |
| | | | | | | | | | | | | | | DG13S1101 |
| | | | | | | | | | | | | | | DG13S166 |
| | | | | | | | | | | | | | | D13S1287 |
| | | | | | | | | | | | | | | DG13S1061 |
| 0.705 | 5.28E−06 | INF | 435 | 0.027 | 0.053 | 721 | 0 | 0 | 0 | 8 | 6 | 0 | 8 | DG13S175 |
| | | | | | | | | | | | | | | DG13S1103 |
| | | | | | | | | | | | | | | DG13S163 |
| | | | | | | | | | | | | | | D13S290 |
| | | | | | | | | | | | | | | DG13S1061 |
| 0.841 | 4.21E−06 | INF | 422 | 0.048 | 0.093 | 721 | 0 | 0 | 0 | 2 | 0 | 0 | 6 | DG13S89 |
| | | | | | | | | | | | | | | DG13S166 |
| | | | | | | | | | | | | | | DG13S163 |
| | | | | | | | | | | | | | | D13S290 |
| | | | | | | | | | | | | | | DG13S1061 |
| 0.767 | 4.02E−06 | 28.11 | 436 | 0.049 | 0.095 | 721 | 0.002 | 0.004 | 0 | 0 | 0 | 2 | 12 | DG13S1101 |
| | | | | | | | | | | | | | | DG13S175 |
| | | | | | | | | | | | | | | DG13S166 |
| | | | | | | | | | | | | | | D13S1287 |
| | | | | | | | | | | | | | | DG13S1061 |
| 0.767 | 1.29E−06 | 31.07 | 436 | 0.047 | 0.092 | 721 | 0.002 | 0.003 | 0 | 0 | 0 | 2 | 12 | DG13S1101 |
| | | | | | | | | | | | | | | DG13S172 |
| | | | | | | | | | | | | | | DG13S166 |
| | | | | | | | | | | | | | | D13S1287 |
| | | | | | | | | | | | | | | DG13S1061 |
| 0.705 | 4.25E−07 | INF | 422 | 0.048 | 0.093 | 721 | 0 | 0 | 0 | 2 | 0 | 0 | 6 | DG13S175 |
| | | | | | | | | | | | | | | DG13S166 |
| | | | | | | | | | | | | | | DG13S163 |
| | | | | | | | | | | | | | | D13S290 |
| | | | | | | | | | | | | | | DG13S1061 |
| 0.683 | 6.58E−06 | INF | 437 | 0.029 | 0.056 | 721 | 0 | 0 | 0 | 4 | 0 | 2 | 14 | DG13S172 |
| | | | | | | | | | | | | | | DG13S1103 |
| | | | | | | | | | | | | | | DG13S166 |
| | | | | | | | | | | | | | | D13S1287 |
| | | | | | | | | | | | | | | DG13S1061 |
| 0.767 | 2.85E−06 | 32.43 | 436 | 0.044 | 0.087 | 721 | 0.001 | 0.003 | 0 | 0 | 0 | 2 | 12 | DG13S1101 |
| | | | | | | | | | | | | | | DG13S166 |
| | | | | | | | | | | | | | | D13S290 |
| | | | | | | | | | | | | | | D13S1287 |
| | | | | | | | | | | | | | | DG13S1061 |
| 0.865 | 9.58E−06 | 18.39 | 451 | 0.023 | 0.045 | 739 | 0.001 | 0.003 | 0 | 0 | 2 | 2 | −16 | D13S289 |
| | | | | | | | | | | | | | | DG13S166 |
| | | | | | | | | | | | | | | DG13S163 |
| | | | | | | | | | | | | | | D13S1287 |
| | | | | | | | | | | | | | | DG13S293 |
| 0.865 | 5.08E−06 | INF | 453 | 0.019 | 0.038 | 739 | 0 | 0 | 0 | 0 | 2 | 0 | −16 | D13S289 |
| | | | | | | | | | | | | | | DG13S166 |
| | | | | | | | | | | | | | | DG13S163 |
| | | | | | | | | | | | | | | DG13S1061 |
| | | | | | | | | | | | | | | DG13S293 |
| 0.927 | 1.02E−07 | 27.65 | 437 | 0.037 | 0.073 | 721 | 0.001 | 0.003 | 4 | 0 | 2 | 14 | 3 | DG13S1103 |
| | | | | | | | | | | | | | | DG13S166 |
| | | | | | | | | | | | | | | D13S1287 |
| | | | | | | | | | | | | | | DG13S1061 |
| | | | | | | | | | | | | | | DG13S301 |

Length = length of haplotype in Mb.
P-val = p-value.
RR = Relative risk.
N af = Number of patients.
P al = allelic frequency of haplotype.
P ca = carrier frequency of haplotype.
N ct = number of controls.
Alleles = alleles in the haplotype.
Markers = markers in the haplotype

Additional haplotypes were associated with MI, as shown in the following Tables.

TABLE 6
shows haplotypes in the FLAP region (FLAP and flanking nucleotide sequences)
that are significantly associated with female MI.

| DG13S1103 | DG00AAFQR | SNP13B_R1028729 | SNP13B_Y1323898 | SNP13B_K912392 | DG00AAFIV | D13S289 | DG13S166 |
|---|---|---|---|---|---|---|---|
| | | | 1 | | 3 | | 0 |
| | | | 1 | | 3 | | 0 |
| | | | 1 | | 3 | | 0 |
| | | | 1 | | 3 | | 0 |
| | | | 1 | | 3 | | 0 |
| | | | 1 | 0 | 3 | | 0 |
| | | | 1 | | 3 | | 0 |
| | | | 1 | | 3 | | 0 |
| | | | 1 | | 3 | | 0 |
| | | | 1 | | 3 | | 0 |
| | | | 1 | | 3 | | 0 |
| | | | 1 | | 3 | | 0 |
| | | | 1 | | 3 | | 0 |
| | | | 1 | | 3 | | 0 |
| | 0 | | 1 | | 3 | | 0 |
| | 0 | | 1 | | 3 | | 0 |
| | 0 | | 1 | | 3 | | 0 |
| | | | 1 | 0 | 3 | | 0 |
| | 0 | | 1 | | 3 | | 0 |
| | 0 | | 1 | 0 | 3 | | 0 |

| DG00AAFJT | DG00AAHII | DG00AAHID | DG00AAHIJ | DG00AAHIH | DG00AAHIE | B_SNP_302524 | B_SNP_302617 | DG00AAHIG |
|---|---|---|---|---|---|---|---|---|
| | | 3 | | 0 | | | 3 | |
| | | 3 | | | | | 3 | |
| | | 3 | 2 | | | | 3 | |
| | | 3 | | | | | 3 | |
| | | 3 | | | | | 3 | |
| | | 3 | | 0 | | | 3 | |
| | | 3 | | | | | 3 | |
| | | | 2 | | | | 3 | |
| | | | 2 | | | | 3 | |
| | 2 | | | | | | 3 | |
| | | | | 0 | | | 3 | |
| | | | | 0 | | | 3 | |
| | | | 2 | 0 | | | 3 | |
| | | | 2 | | | | 3 | |
| | | | | | | | 3 | |
| | | 3 | | | | | 3 | |
| | | | 2 | | | | 3 | |
| | | | | 0 | | | 3 | |
| | | | | | | | 3 | |

| DG00AAHIF | DG00AAHOI | D13S1238 | DG13S2605 | DG13S163 | p-val | N_aff | aff.frq | N_ctrl | ctrl.frq | rel_risk | PAR | info |
|---|---|---|---|---|---|---|---|---|---|---|---|---|
| | 2 | −2 | | | 1.30E−05 | 455 | 0.108 | 811 | 0.048 | 2.4 | 0.122 | 0.615 |
| | | −2 | 0 | | 7.61E−06 | 455 | 0.065 | 812 | 0.02 | 3.45 | 0.091 | 0.615 |
| | | −2 | 0 | | 8.82E−06 | 455 | 0.065 | 812 | 0.02 | 3.47 | 0.092 | 0.602 |
| | | −2 | 0 | | 9.31E−06 | 455 | 0.065 | 812 | 0.02 | 3.39 | 0.089 | 0.611 |
| | 2 | −2 | 0 | | 6.91E−06 | 455 | 0.063 | 812 | 0.019 | 3.54 | 0.09 | 0.624 |
| | | −2 | 0 | | 9.76E−06 | 455 | 0.063 | 812 | 0.019 | 3.51 | 0.089 | 0.606 |
| | 2 | −2 | | | 1.09E−05 | 455 | 0.063 | 811 | 0.019 | 3.41 | 0.086 | 0.618 |
| 2 | | −2 | 0 | | 1.10E−05 | 455 | 0.063 | 812 | 0.019 | 3.44 | 0.087 | 0.611 |
| | 2 | −2 | 0 | | 1.11E−05 | 455 | 0.063 | 812 | 0.018 | 3.56 | 0.086 | 0.589 |
| | 2 | −2 | | | 1.22E−05 | 455 | 0.063 | 811 | 0.018 | 3.6 | 0.067 | 0.577 |
| | 2 | −2 | 0 | | 1.26E−05 | 455 | 0.063 | 812 | 0.02 | 3.35 | 0.068 | 0.629 |
| | 2 | −2 | 0 | | 8.59E−06 | 455 | 0.062 | 812 | 0.018 | 3.53 | 0.065 | 0.62 |
| | 2 | −2 | | | 1.20E−05 | 455 | 0.062 | 811 | 0.019 | 3.42 | 0.086 | 0.617 |
| | 2 | −2 | | | 1.21E−05 | 455 | 0.062 | 811 | 0.019 | 3.43 | 0.086 | 0.619 |
| | 2 | −2 | | | 7.93E−05 | 455 | 0.061 | 811 | 0.016 | 3.95 | 0.088 | 0.582 |
| | 2 | −2 | | | 1.09E−05 | 455 | 0.061 | 811 | 0.017 | 3.85 | 0.09 | 0.56 |
| | 2 | −2 | | | 5.00E−06 | 455 | 0.06 | 811 | 0.015 | 4.11 | 0.087 | 0.576 |
| | 2 | −2 | | | 1.31E−05 | 455 | 0.06 | 811 | 0.017 | 3.66 | 0.085 | 0.586 |
| | 2 | −2 | | | 8.53E−06 | 455 | 0.059 | 811 | 0.016 | 3.85 | 0.085 | 0.593 |
| | 2 | −2 | | | 9.63E−06 | 455 | 0.058 | 811 | 0.015 | 4.03 | 0.085 | 0.565 |

TABLE 7
Two variants of the female MI “at risk” haplotypes

| DG13S1103 | DG00AAFQR | SNP13B_R1028729 | SNP13B_Y1323898 | SNP13B_K912392 | DG00AAFIV | D13S289 | DG13S166 |
|---|---|---|---|---|---|---|---|
| female MI | | | | | | | |
| | 0 | | 1 | | 3 | | 0 |
| | | | 1 | | 3 | | 0 |

| DG00AAFJT | DG00AAHII | DG00AAHID | DG00AAHIJ | DG00AAHIH | DG00AAHIE | B_SNP_302524 | B_SNP_302617 | DG00AAHIG |
|---|---|---|---|---|---|---|---|---|
| | | 3 | | | | | 3 | |

| DG00AAHIF | DG00AAHOI | D13S1238 | DG13S2605 | DG13S163 | p-val | N_aff | aff.frq | N_ctrl | ctrl.frq | rel_risk | PAR | info |
|---|---|---|---|---|---|---|---|---|---|---|---|---|
| | 2 | −2 | | | 6.38E−06 | 454 | 0.059 | 809 | 0.015 | 4.05 | 0.086 | 0.577 |
| | 2 | −2 | | | 2.74E−05 | 447 | 0.106 | 809 | 0.048 | 2.33 | 0.116 | 0.623 |

P-val: p-value for the association.
N_aff: Number of patients used in the analysis.
Aff.frq: haplotype frequency in patients.
N_ctrl: number of controls used in the analysis.
Ctrl.frq: Haplotype frequency in controls.
Rel_risk: Relative risk of the haplotype.
PAR: population attributable risk.
Info: information content.

TABLE 8
The frequencies of the female MI “at risk” haplotypes in male patients vs controls.

| DG13S1103 | DG00AAFQR | SNP13B_R1028729 | SNP13B_Y1323898 | SNP13B_K912392 | DG00AAFIV | D13S289 | DG13S166 |
|---|---|---|---|---|---|---|---|
| male MI | | | | | | | |
| | 0 | | 1 | | 3 | | 0 |
| | | | 1 | | 3 | | 0 |

| DG00AAFJT | DG00AAHII | DG00AAHID | DG00AAHIJ | DG00AAHIH | DG00AAHIE | B_SNP_302524 | B_SNP_302617 | DG00AAHIG |
|---|---|---|---|---|---|---|---|---|
| | | 3 | | | | | 3 | |

| DG00AAHIF | DG00AAHOI | D13S1238 | DG13S2605 | DG13S163 | p-val | N_aff | aff.frq | N_ctrl | ctrl.frq | rel_risk | PAR | info |
|---|---|---|---|---|---|---|---|---|---|---|---|---|
| | 2 | −2 | | | 3.37E−01 | 1087 | 0.027 | 809 | 0.021 | 1.32 | 0.013 | 0.577 |
| | 2 | −2 | | | 5.39E−01 | 1067 | 0.056 | 809 | 0.05 | 1.13 | 0.013 | 0.568 |

P-val: p-value for the association.
N_aff: Number of patients used in the analysis.
Aff.frq: haplotype frequency in patients.
N_ctrl: number of controls used in the analysis.
Ctrl.frq: Haplotype frequency in controls.
Rel_risk: Relative risk of the haplotype.
PAR: population attributable risk.
Info: information content.

TABLE 9
The selected SNP haplotypes and the corresponding p-values, relative risk (RR), number of
patients (#aff), allelic frequency in patients (aff.frq.), carrier frequency in patients (carr.frq.), number
of controls (#con), allelic frequency in controls (con.frq.), population attributable risk (PAR). The
patients used for this analysis were all unrelated within 4 meioses.

| | p-val | RR | #aff | aff.frq. | carr.frq. | #con | con.frq. | PAR | DG00AAFIU | SG13S25 | DG00AAJFF |
|---|---|---|---|---|---|---|---|---|---|---|---|
| B4 | 4.80E−05 | 2.08 | 903 | 0.106 | 0.2 | 619 | 0.054 | 0.11 | | 2 | |
| B5 | 2.40E−05 | 2.2 | 910 | 0.101 | 0.19 | 623 | 0.049 | 0.11 | 3 | 2 | |
| B6 | 1.80E−06 | 2.22 | 913 | 0.131 | 0.24 | 623 | 0.063 | 0.14 | 3 | 2 | 2 |
| A4 | 5.10E−06 | 1.81 | 919 | 0.159 | 0.29 | 623 | 0.095 | 0.14 | | 2 | |
| A5 | 2.60E−06 | 1.91 | 920 | 0.15 | 0.28 | 624 | 0.085 | 0.14 | 3 | 2 | |

| | DG00AAHII | DG00AAHID | B_SNP_310657 | SG13S30 | SG13S32 | SG13S42 | SG13S35 |
|---|---|---|---|---|---|---|---|
| B4 | 2 | | | 2 | | 0 | |
| B5 | 2 | | | 2 | | 0 | |
| B6 | 2 | | | | 0 | | 2 |
| A4 | | 3 | 2 | | 0 | | |
| A5 | | 3 | 2 | | 0 | | |

### EXAMPLE 2

### Relationship Between Mutation in 5-LO Promoter and MI

A family of mutations in the G-C rich transcription factor binding region of the 5-LO gene has previously been identified. These mutations consist of deletion of one, deletion of two, or addition of one zinc finger (Sp1/Egr-1) binding sites in the region 176 to 147 bp upstream from the ATG translation start site where there are normally 5 Sp1 binding motifs in tandem. These naturally occurring mutations in the human 5-LO gene promoter have been shown to modify transcription factor binding and reporter gene transcription. The capacity of the mutant forms of DNA to promote transcription of CAT reporter constructs have been shown to be significantly less than that of the wild type DNA (J. Clin. Invest. Volume 99, Number 5, March 1997, 1130-1137).

To test whether 5-LO is associated with the atherosclerotic diseases, particularly myocardial infarction (MI) in the human population, this promoter polymorphism, consisting of variable number of tandem Sp1/Egr-1 binding sites, was genotyped in 1112 MI patients, 748 patients with PAOD, and 541 stroke patients.

The results, shown in Table 10, demonstrate that the wild type allele (which represents the allele with the most active promoter and thus with the highest expression of the 5-LO mRNA) is significantly associated with MI (RR=1.2, p<0.05). The results are consistent with a disease hypothesis that increased expression of the 5-LO plays a role in the pathogenesis of MI.

TABLE 10

| | | | | | Risk | |
|---|---|---|---|---|---|---|
| | N_aff | Frq_aff | N_ctrl | Frq_ctrl | Ratio | P-value |
| MI patients | 1112 | 0.8701 | 734 | 0.8501 | 1.1803 | 0.048 |
| Independent | 969 | 0.8720 | 734 | 0.8501 | 1.2013 | 0.037 |
| Males | 646 | 0.8740 | 734 | 0.8501 | 1.2232 | 0.039 |
| Females | 465 | 0.8645 | 734 | 0.8501 | 1.1249 | 0.180 |
| Age of | 522 | 0.8745 | 734 | 0.8501 | 1.2286 | 0.046 |
| onset <60 | | | | | | |
| Males | 353 | 0.8768 | 734 | 0.8501 | 1.2542 | 0.053 |
| Females | 169 | 0.8698 | 734 | 0.8501 | 1.1779 | 0.202 |

### EXAMPLE 3

### Elevated LTE4 Biosynthesis in Individuals with the Flap MI-Risk Haplotype

Based on the known function of the end products of the leukotriene pathway and based on our 5-LO association data, the association of the FLAP haplotype with MI is best explained by increased expression and/or increased function of the FLAP gene. In other words, those individuals that have a “at risk” FLAP haplotype have either, or both, increased amount of FLAP, or more active FLAP. This would lead to increased production of leukotrienes in these individuals.

To demonstrate this theory, LTE4, a downstream leukotriene metabolite, was measured in patient serum samples. A quantitative determination of LTE4 in human serum was performed by liquid chromatography coupled with tandem mass spectrometry. The protocol was performed as follows:

Analytical Method

TABLE P1
(Protocol 1): List of Abbreviations

| | |
|---|---|
| CAN | Acetonitrile |
| IS | Internal standard |
| LC-MS/MS | Liquid chromatography tandem mass spectrometry |
| LOQ | Limit of quantification |
| QCs | Quality controls |
| R² | Coefficient of determination |
| SS | Spiking solution |

Apparatus and Conditions

TABLE P2
Analytical apparatus and conditions

| Instruments/Conditions | Details | | | |
|---|---|---|---|---|
| Analytical column | Zorbax extend C_{18,} 3.5 μm (50 × 2.1 mm) | | | |
| Column temperature | Ambient | | | |
| Pump and flow | Hewlett Packard Series 1100 Binary pump delivering 0.3 ml/min | | | |
| Mobile phase | A: Buffer: Acetonitrile: H₂O (5:95% v/v). (Containing 10 mM | | | |
| | Ammonium Acetate and 0.1% Acetic acid at pH 4.6). | | | |
| | B: Buffer: Acetonitrile: H₂O (95:5% v/v). (Containing 10 mM | | | |
| | Ammonium Acetate and 0.1% Acetic acid at pH 4.6). | | | |
| Gradient | Time | % A | % B | Flow rate |
| | 0.00 | 30 | 70 | 0.3 ml/min |
| | 1.00 | 30 | 70 | 0.3 ml/min |
| | 1.50 | 90 | 10 | 0.3 ml/min |
| | 6.00 | 90 | 10 | 0.3 ml/min |
| | 6.50 | 30 | 70 | 0.3 ml/min |
| | 10.00 | 30 | 70 | 0.3 ml/min |

| Sample injection | HTC PAL autosampler 10 μl onto the HPLC column | | |
|---|---|---|---|
| Mass Spectrometric | Quattro Ultima ™ Tandem MS/MS, Micromass. England. | | |
| system | | | |
| Recording and | Mass Lynx, version 3.5. All chromatograms and reports are | | |
| integration | printed out in hardcopy and stored in electronic form on the | | |
| | workstation hard disk drive. Recording time was 10 min. | | |
| Retentions times | LTE₄ ˜ 3.05 min. | | |
| | LTE₄-d₃ ˜ 3.05 min. | | |
| Ionization mode | Electrospray atmospheric pressure in negative ion mode | | |
| Scan mode | Multiple reaction monitoring (MRM) | | |
| | Compound | Parent ion | Daughter ion |
| | LTE₄ | 438.2 | 333.2 |
| | LTE₄-d₃ | 441.2 | 336.2 |

Other Instruments

TABLE P3
The apparatus used for sample treatment and measurements

| Apparatus | Brand | Type |
|---|---|---|
| Pipette | Eppendorf | Edos 5221 |
| Pipette | Labsystems | Finnpipette 200 μl |
| Centrifuge | Eppendorf | 5417C |
| Evaporation unit | Porvair | Ultravap |
| Vibrofix | Ika-Werk | VF-1 |
| | Thermolyne | Maxi-mix III ™, 65800 |
| Balance | Sartorius | LA 120 S |
| Ultra sonic bath | Cole Parmer | 8891 |

Materials

TABLE P4
Reagents for sample treatment and measurements

| Reagent | Manufacturer | Quality | Art no. |
|---|---|---|---|
| Acetonitrile (ACN) | Rathburn | HPLC grade | RH 1016 |
| Methanol | Rathburn | HPLC grade | RH 1019 |
| Ammonium acetate | Merck | Pro analysis | 1116 |

TABLE P5
Reference substances

| | Details | Reference |
|---|---|---|
| Reference | Leukotrine E₄ from Cayman Chemical, MI, | 20410 |
| standards | USA | |
| Internal | Leukotriene E₄-20, 20, 20-d₃ from Biomol, PA, | S10120 |
| standards | USA | |

Stock Solutions

A stock solution of LTE₄ was prepared by the supplier at a concentration of 100 μg/ml in methanol. The stock solution was diluted to a concentration of 20 μg/ml in methanol and this working solution (WS-1) was kept refrigerated at 2-8° C.

An internal standard stock solution (LTE₄-d₃) was prepared by the supplier at concentration of 49.5 μg/ml. The stock solution was diluted to a concentration of 1 μg/ml in methanol and this working solution was kept refrigerated at 2-8° C.

Preparation of Spiking Solutions, Calibration Standards and Quality Control Samples

Spiking solutions (SS) in the concentration range of 1 ng/ml to 10000 ng/ml were prepared by dilution of the working Solution. The following spiking solutions were prepared:

TABLE P6
Spiking solutions for calibration standards

| | Concen- | |
|---|---|---|
| | tration | |
| SS | (ng/ml) | Preparation |
| 1 | 10000 | 500 μl of WS-1 (20 μg/ml) diluted to 1.0 ml with 70% |
| | | MeOH/water |
| 2 | 1000 | 100 μl of SS-1 was diluted to 1.0 ml with 70% |
| | | MeOH/water |
| 3 | 100 | 100 μl of SS-2 was diluted to 1.0 ml with 70% |
| | | MeOH/water |
| 4 | 30 | 300 μl of SS-3 was diluted to 1.0 ml with 70% |
| | | MeOH/water |
| 5 | 20 | 200 μl of SS-3 was diluted to 1.0 ml with 70% |
| | | MeOH/water |
| 6 | 16 | 160 μl of SS-3 was diluted to 1.0 ml with 70% |
| | | MeOH/water |
| 7 | 12 | 120 μl of SS-3 was diluted to 1.0 ml with 70% |
| | | MeOH/water |
| 8 | 8.0 | 400 μl of SS-5 was diluted to 1.0 ml with 70% |
| | | MeOH/water |
| 9 | 4.0 | 200 μl of SS-5 was diluted to 1.0 ml with 70% |
| | | MeOH/water |
| 10 | 2.0 | 100 μl of SS-5 was diluted to 1.0 ml with 70% |
| | | MeOH/water |
| 11 | 1.4 | 175 μl of SS-8 was diluted to 1.0 ml with 70% |
| | | MeOH/water |
| 12 | 1.0 | 125 μl of SS-8 was diluted to 1.0 ml with 70% |
| | | MeOH/water |

TABLE P7
Spiking solutions for quality controls

| | Concentration | |
|---|---|---|
| SS | (ng/ml) | Preparation |
| 13 | 14 | 140 μl of SS-3 was diluted to 1.0 ml with 70% |
| | | MeOH/water |
| 14 | 6.0 | 300 μl of SS-5 was diluted to 1.0 ml with 70% |
| | | MeOH/water |
| 15 | 2.4 | 120 μl of SS-5 was diluted to 1.0 ml with 70% |
| | | MeOH/water |

After preparation, spiking solutions for calibration standards and quality controls were kept refrigerated at 2-8° C.

Preparation of Calibration Standards and Quality Controls

Fresh calibration standards and quality controls (QCs) were prepared each day by spiking blank plasma as described in Tables P8 and P9, respectively.

TABLE P8
Preparation of calibration standards

| Concentration | | |
|---|---|---|
| (ng/ml) | SS (μl) | Blank Plasma |
| 1500 | 20 μl of the SS-4 (30 ng/ml) | 380 μl |
| 1000 | 20 μl of the SS-5 (20 ng/ml) | 380 μl |
| 800 | 20 μl of the SS-6 (16 ng/ml) | 380 μl |
| 600 | 20 μl of the SS-7 (12 ng/ml) | 380 μl |
| 400 | 20 μl of the SS-8 (8 ng/ml) | 380 μl |
| 200 | 20 μl of the SS-9 (4.0 ng/ml) | 380 μl |
| 100 | 20 μl of the SS-10 (2.0 ng/ml) | 380 μl |
| 70 | 20 μl of the SS-11 (1.4 ng/ml) | 380 μl |
| 50 | 20 μl of the SS-12 (1.0 ng/ml) | 380 μl |

TABLE P9
Preparation of quality controls

| Concentration | | |
|---|---|---|
| (ng/ml) | SS (μl) | Blank Plasma |
| 800 | 20 μl of the SS-13 (14 ng/ml) | 380 μl |
| 40 | 20 μl of the SS-14 (6.0 ng/ml) | 380 μl |
| 8.0 | 20 μl of the SS-15 (2.4 ng/ml) | 380 μl |

Sample Preparation

Aliquots of 400 μl of each study sample, calibration standards, QC samples and control blank are pipetted into an eppendorf vial. All samples apart from blank are then spiked with 20 μl of internal standard working solution and the samples are then vortex-mixed for few seconds. The pH of the plasma samples is adjusted to pH 4.5 using 60 μl of 10% acetic acid and centrifuged for 10 min. at 4100 rpm immediately before the extraction. The solid phase extraction 96-well plate is conditioned with 1 ml methanol and 1 ml water. Then 400 μl of the plasma samples are loaded on the plate. Vacuum is applied, followed by drying the disk for 1 min. After being washed with 2 ml water and 1 ml 30% methanol in 2% acetic acid. Next the plate is eluted with 0.6 ml methanol. The plate is then dried for few minutes. The methanol eluate is evaporated almost to dryness under a stream of nitrogen at 45° C. The residue is reconstituted in 30 μl mobile phase and vortex-mixed for few min. Subsequently, the solutions are centrifuged for 10 min at 10,000 rpm. and 10 μl are injected by the autosampler into the LC-MS/MS system for quantification.

Performance of Measurements

The samples will be prepared and measured in batches and a typical batch will consist of:

One set of calibration standards, one extra lowest calibration standard and one blank. Samples collected from a 16 individuals and one set of control samples (C_{L}, C_{M}, C_{H}) Samples collected from a 17 individuals and one set of control samples (C_{L}, C_{M}, C_{H})

Quantitative Determination of Analyte in Plasma Samples

The standard curve is calculated from the peak area ratios ANALYTE/INTERNAL STANDARD of the calibration standards and their nominal ANALYTE concentrations. The unknown samples for LTE₄ were calculated from a quadratic regression equation where a weighted curve, 1/X², is used. The measured peak area of the samples was converted into pictogram of ANALYTE per milliliter (pg/ml) of plasma according to the calculated equation for the standard curve.

The calculation of the regression for the standard curve and the calculations of the concentration of the unknown samples and the control samples are performed with MassLynx Software.

Acceptance Criteria

Calibration Standards

The coefficient of determination (R²) for the calibration curve must exceed 0.98.

The calibration curve included the concentration range from 50 pg/ml-1500 pg/ml.

Concentration of the calibration standards must be within ±25% of their nominal value when recalculated from the regression equation. Calibration standards that fail these criteria (at most 3 in each run) are rejected and the calibration performed again with the remaining standards. If the standard curve is not accepted, the samples must be reanalyzed.

Control Samples

At least two thirds of the analysed quality controls must be within ±25% of their nominal value when calculated from regression equation. If more than a third of the controls fail, the samples must be reanalyzed.

Results

Table 11 (below) shows that the female MI “at risk” haplotype is more significantly associated with female MI patients who have high LTE4 levels (top 50th percentile), than with female MI patients who have low levels of LTE4 (bottom 50th percentile).

In addition, haplotype analysis, comparing female MI patients with high levels of LTE4 with female patients with low levels, showed that those with high levels had increased prevalence of the “at risk” haplotype by 1.6 fold (see Table 12). The results show clearly that the “at risk” haplotypes are enriched in the MI patient group that has high levels of LTE4. The carrier frequency of the “at risk” haplotypes are 12% and 20%, respectively, in the whole female MI group, but go up to 15% and 24%, respectively, in the female MI group that has high levels of LTE4. Correspondingly, the carrier frequency of the “at risk” haplotypes decrease to 8% and 18%, respectively, in the group of female MI that has low levels of LTE4 (Note carrier frequencies are twice the disease allele frequency times 1 minus the disease allele frequency plus the square of the disease allele frequency).

Note that LTE4 may simply reflect the leukotriene synthesis rate of the leukotriene synthetic pathway upstream of the key leukotriene metabolite involved in MI risk. For example, leukotriene B4 is probably more likely than leukotriene E4 to be involved in the inflammatory aspects of MI plaques but since B4 has a short half life, it is difficult to measure reliably in serum samples, while E4 has long term stability.

TABLE 11
Association of the at risk haplotypes for female MI, with female MI who also have high
levels of LTE4 (>50 pg/ml (roughly the upper 50th percentile). Less significant association between
the at risk haplotype for female MI, with female MI who also have low levels of LTE4 (<50 pg/ml).

| DG13S1103 | DG00AAFQR | SNP13B_R1028729 | SNP13B_Y1323898 | SNP13B_K912392 | DG00AAFIV | D13S289 | DG13S166 |
|---|---|---|---|---|---|---|---|
| High LTE4 | | | | | | | |
| | 0 | | 1 | | 3 | | 0 |
| | | | 1 | | 3 | | 0 |
| Low LTE4 | | | | | | | |
| | 0 | | 1 | | 3 | | 0 |
| | | | 1 | | 3 | | 0 |

| | | | | | | | | |
|---|---|---|---|---|---|---|---|---|
| DG00AAFJT | DG00AAHII | DG00AAHID | DG00AAHIJ | DG00AAHIH | DG00AAHIE | B_SNP_302524 | B_SNP_302617 | DG00AAHIG |
| | | 3 | | | | | 3 | |
| | | 3 | | | | | 3 | |

| DG00AAHIF | DG00AAHOI | D13S1238 | DG13S2605 | DG13S163 | p-val | N_aff | aff.frq | N_ctrl | ctrl.frq | rel_risk | PAR | info |
|---|---|---|---|---|---|---|---|---|---|---|---|---|
| | 2 | −2 | | | 3.72E−06 | 221 | 0.075 | 809 | 0.014 | 5.51 | 0.115 | 0.565 |
| | 2 | −2 | | | 2.30E−05 | 220 | 0.122 | 809 | 0.046 | 2.89 | 0.154 | 0.608 |
| | 2 | −2 | | | 4.65E−02 | 185 | 0.04 | 809 | 0.015 | 2.67 | 0.048 | 0.511 |
| | 2 | −2 | | | 2.88E−02 | 182 | 0.087 | 809 | 0.048 | 1.89 | 0.08 | 0.622 |

P-val: p-value for the association.
N_aff: Number of patients used in the analysis.
Aff.frq: haplotype frequency in patients.
N_ctrl: number of controls used in the analysis.
Ctrl.frq: Haplotype frequency in controls.
Rel_risk: Relative risk of the haplotype.
PAR: population attributable risk.
Info: information content.

TABLE 12
Association between haplotypes that are most significantly associated with female MI, and
serum LTE4 levels. Here, the group of affected individuals are defined as female MI patients with high serum
LTE4 (higher than 50 pg/ml) and the control group is defined as female MI patients with low serum LTE4
(below 50 pg/ml)

| DG13S1103 | DG00AAFQR | SNP13B_R1028729 | SNP13B_Y1323898 | SNP13B_K912392 | DG00AAFIV | D13S289 | DG13S166 |
|---|---|---|---|---|---|---|---|
| | High vs low LTE4 | | | | | | |
| | 0 | | 1 | | 3 | | 0 |
| | | | 1 | | 3 | | 0 |

| DG00AAFJT | DG00AAHII | DG00AAHID | DG00AAHIJ | DG00AAHIH | DG00AAHIE | B_SNP_302524 | B_SNP_302617 | DG00AAHIG |
|---|---|---|---|---|---|---|---|---|
| | | 3 | | | | | 3 | |

| DG00AAHIF | DG00AAHOI | D13S1238 | DG13S2605 | DG13S163 | p-val | N_aff | aff.frq | N_ctrl | ctrl.frq | rel_risk | PAR | info |
|---|---|---|---|---|---|---|---|---|---|---|---|---|
| | 2 | −2 | | | 1.61E−01 | 221 | 0.084 | 185 | 0.054 | 1.61 | 0.063 | 0.689 |
| | 2 | −2 | | | 1.20E−01 | 220 | 0.13 | 182 | 0.088 | 1.54 | 0.089 | 0.686 |

P-val: p-value for the association.
N_aff: Number of patients used in the analysis.
Aff.frq: haplotype frequency in patients.
N_ctrl: number of controls used in the analysis.
Ctrl.frq: Haplotype frequency in controls.
Rel_risk: Relative risk of the haplotype.
PAR: population attributable risk.
Info: information content.

### EXAMPLE 4

### Elevated LTE4 Correlated with Elevated C-Reactive Protein (CRP)

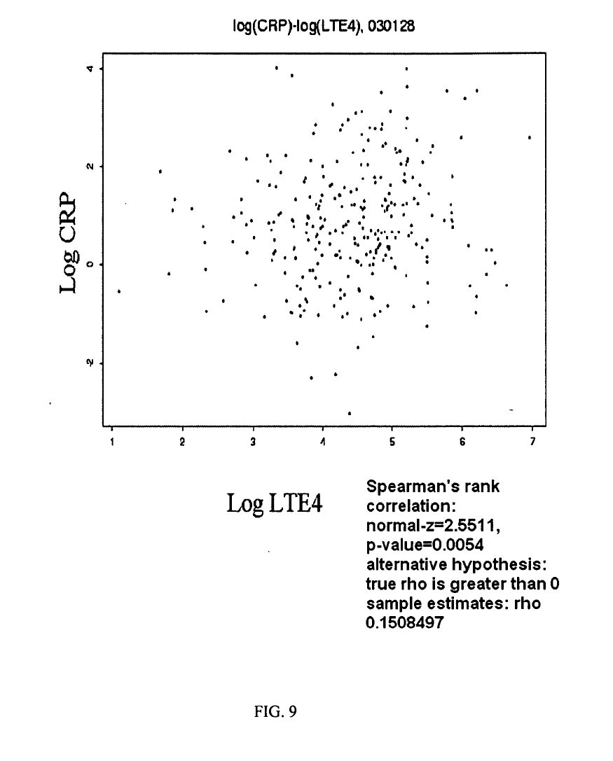

The relationship between the increased production of leukotrienes and the inflammatory marker CRP, a well established risk factor for MI, was then explored. As shown in FIG. 9, a significant positive correlation was found between serum LTE4 levels and serum CRP levels.

### EXAMPLE 5

### Assessment of Level of CRP in Patients with At-Risk Haplotype

The level of CRP in female patients with female MI at-risk haplotypes was assessed, in order to demonstrate the presence of a raised level of inflammatory marker in the presence of the female MI at-risk haplotype. Results are shown in Table 13. The average CRP was elevated in those patients with the at-risk haplotype versus those without it.

TABLE 13

| All female | | | | |
|---|---|---|---|---|
| patients | | no | Mean CRP | SE CRP |
| affecteds: | With haplotype. | 155 | 4.91 | 8.7 |
| | Not with haplotype. | 218 | 4.35 | 6.13 |

### EXAMPLE 6

### Elevated Serum LTE4 Levels in MI Patients Versus Controls

The end products of the leukotriene pathway are potent inflammatory lipid mediators that can potentially contribute to development of atherosclerosis and destabilization of atherosclerotic plaques through lipid oxidation and/or proinflammatory effects. Examples one through five show that: 1) MI correlates with genetic variation at FLAP; 2) MI correlates with high expression promoter polymorphism at 5-LO; 3) C-reactive protein levels correlate with serum leukotriene E4; and 4) Patients with MI-risk FLAP haplotypes have higher levels of serum leukotriene E4 and CRP. Based on these data, it was hypothesized that serum leukotriene E4 levels correlate with MI risk.

To test this hypothesis, LTE4, a downstream leukotriene metabolite, was measured in 488 female MI patient and 164 control serum samples. The LTE4 levels for the patients was higher than that for the controls using a one-sided Wilcoxon rank-sum test. The p-value of the difference was 0.0092 thus confirming our hypothesis. Therefore, elevated leukotriene E4 represents a risk factor for MI. Serum or plasma LTE4 levels may be used to profile the MI risk for individuals to aid in deciding which treatment and lifestyle management plan is best for primary or secondary MI prevention. In the same way other leukotriene metabolites may be used to risk profile for MI.

### EXAMPLE 7

### Increased LTB4 Production in Activated Neutrophils form MI Patients

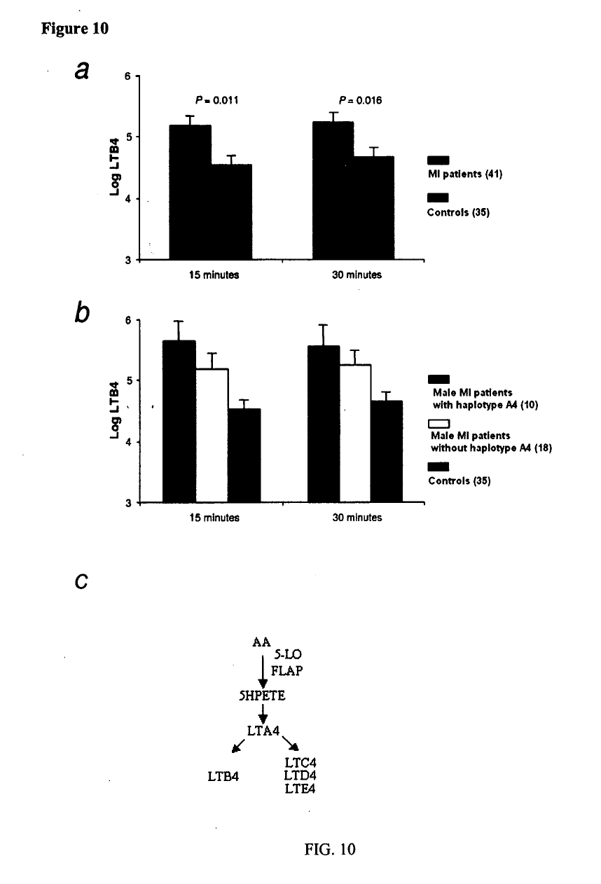

A principal bioactive product of one of the two branches of the 5-LO pathway is LTB4. To determine whether the patients with past history of MI have increased activity of the 5-LO pathway compared to controls, we measured the LTB4 production in isolated blood neutrophils before and after stimulation in vitro with the calcium ionophore, ionomycin. No difference was detected between the LTB4 production in resting neutrophils from MI patients or controls (results not shown). In contrast, the LTB4 generation by neutrophils from MI patients stimulated with the ionophore was significantly greater than by neutrophils from controls at 15 and 30 minutes, respectively (FIG. 10a). Moreover, as shown in FIG. 10b, the observed increase in the LTB4 release was largely accounted for by male carriers of haplotype A4, whose cells produced significantly more LTB4 than cells from controls (P value=0.0042) (Table 14). As shown in Table 14 there was also a heightened LTB4 response in males who do not carry HapA but of borderline significance. This could be explained by additional variants in the FLAP gene that have not been uncovered, or alternatively in other genes belonging to the 5-LO pathway, that may account for upregulation in the LTB4 response in some of the patients without the FLAP at-risk haplotype. As shown in Table 14, we did not detect differences in LTB4 response in females. However, due to a small sample size this cannot be considered conclusive. Taken together, the elevated levels of LTB4 production of stimulated neutrophils from male carriers of the at-risk haplotype suggest that the disease associated variants in the FLAP gene increase FLAP's response to factors that stimulate inflammatory cells, resulting in increased leukotriene production and increased risk for MI.

Methods

Isolation and Activation of Peripheral Blood Neutrophils

50 ml of blood were drawn into EDTA containing vacutainers from 43 MI patients and 35 age and sex matched controls. All blood was drawn at the same time in the early morning after 12 hours of fasting. The neutrophils were isolated using Ficoll-Paque PLUS (Amersham Biosciences).

Briefly, the red cell pellets from the Ficoll gradient were harvested and red blood cells subsequently lysed in 0.165 M NH₄CL for 10 minutes on ice. After washing with PBS, neutrophils were counted and plated at 2×10⁶ cells/ml in 4 ml cultures of 15% Fetal calf serum (FCS) (GIBCO BRL) in RPMI-1640 (GIBCO BRL). The cells were then stimulated with maximum effective concentration of ionomycin (1 μM). At 0, 15, 30, 60 minutes post inomycin addition 600 μl of culture medium was aspirated and stored at −80 C for the measurement of LTB4 release as described below. The cells were maintained at 37° C. in a humidified atmosphere of 5% CO₂/95% air. We treated all samples with indomethasine (1 μM) to block the cyclooxygenase enzyme.

Ionomycin-Induced Release of LTB4 in Neutrophils

LTB4 Immunoassay (R&D systems) was used to quantitate LTB4 concentration in supernatant from cultured inomycin stimulated neutrophils. The assay used is based on the competitive binding technique in which LTB4 present in the testing samples (200 μl) competes with a fixed amount of alkaline phosphatase-labelled LTB4 for sites on a rabbit polyclonal antibody. During the incubation, the polyclonal Ab becomes bound to a goat anti-rabbit Ab coated onto the microplates. Following a wash to remove excess conjugate and unbound sample, a substrate solution is added to the wells to determine the bound enzyme activity. The color development is stopped and the absorbance is read at 405 nm. The intensity of the color is inversely proportional to the concentration of LTB4 in the sample. Each LTB4 measurement using the LTB4 Immunoassay, was done in duplicate.

TABLE 14
LTB4 levels after ionomycin stimulation of isolated neutrophils^{a}

| | After 15 Minutes | | After 30 Minutes | |
|---|---|---|---|---|
| Phenotype (n) | Mean (SD) | P value | Mean (SD) | P value |
| Controls (35) | 4.53 (1.00) | | 4.67 (0.88) | |
| Males (18) | 4.61 (1.10) | | 4.68 (1.07) | |
| Females (17) | 4.51 (0.88) | | 4.67 (0.62) | |
| MI (41) | 5.18 (1.09) | 0.011 | 5.24 (1.06) | 0.016 |
| Carriers (16) | 5.26 (1.09) | 0.027 | 5.27 (1.09) | 0.051 |
| Non-carriers (24) | 5.12 (1.08) | 0.040 | 5.22 (1.03) | 0.035 |
| MI males (28) | 5.37 (1.10) | 0.0033 | 5.38 (1.09) | 0.0076 |
| Carriers (10) | 5.66 (1.04) | 0.0042 | 5.58 (1.12) | 0.013 |
| Non-carriers (18) | 5.20 (1.09) | 0.039 | 5.26 (1.05) | 0.041 |
| MI females (13) | 4.78 (0.95) | 0.46 | 4.95 (0.92) | 0.36 |
| Carriers (6) | 4.59 (0.80) | 0.90 | 4.75 (0.82) | 0.85 |
| Non-carriers (7) | 4.94 (1.04) | 0.34 | 5.12 (0.96) | 0.25 |
| ^{a}Mean ± SD of log-transformed values of LTB4 levels of ionomycin-stimulated neutrophils from MI patients and controls. Results are shown for two time points: 15 and 30 minutes. The results for males and females and for MI male and female carriers and non-carriers of the at-risk haplotype HapA are shown separately. Two-sided | | | | |
| # p values corresponding to a standard two-sample test of the difference in the mean values between the MI patients, their various sub-cohorts and the controls are shown. | | | | |

### EXAMPLE 8

### Haplotypes Associated with MI Also Confer Risk of Stroke and PAOD

Because stroke and PAOD are diseases that are closely related to MI (all occur on the basis of atherosclerosis), we examined if the SNP haplotype in the FLAP gene that confers risk to MI also conferred risk of stroke and/or PAOD. The ‘at risk’ haplotype can be defined by the following 4 SNPs: SG13S25 with allele G,

DG00AAHID with Allele T, B_SNP_{—}310657 with Allele G, and SG13S32 with Allele A.

Table 15 shows that the haplotype (A4) increases the risk of having a stroke to a similar extent as it increases the risk of having an MI. The ‘at risk’ haplotype is carried by 28% of stroke patients and 17% of controls, meaning that the relative risk of having stroke for the carriers of this haplotype is 1.7 (p-value=5.8 10⁻⁰⁶). Although not as significant, the ‘at risk’ haplotype also confers risk of having PAOD.

TABLE 15

| | | p-val | r | #aff | aff.frq. | #con | con.frq. | info | SG13S6 | SG13S25 | DG00AAJFF | DG00AAFJT |
|---|---|---|---|---|---|---|---|---|---|---|---|---|
| MI haplotypes | | | | | | | | | | | | |
| All MI patients | A4 | 5.3E−07 | 1.80 | 1407 | 0.16 | 614 | 0.09 | 0.82 | | 2 | | |
| | B4 | 1.0E−04 | 1.87 | 1388 | 0.10 | 612 | 0.06 | 0.67 | | 2 | | |
| Males MI | A4 | 2.5E−08 | 2.00 | 864 | 0.17 | 614 | 0.09 | 0.82 | | 2 | | |
| | B4 | 1.1E−05 | 2.12 | 852 | 0.11 | 612 | 0.06 | 0.67 | | 2 | | |
| Females MI | A4 | 1.9E−02 | 1.44 | 543 | 0.13 | 614 | 0.09 | 0.73 | | 2 | | |
| | B4 | 7.9E−02 | 1.45 | 536 | 0.08 | 612 | 0.06 | 0.60 | | 2 | | |
| Replication in stroke | | | | | | | | | | | | |
| All stroke | A4 | 5.8E−06 | 1.73 | 1238 | 0.15 | 614 | 0.09 | 0.80 | | 2 | | |
| patients | B4 | 2.3E−04 | 1.83 | 1000 | 0.10 | 612 | 0.06 | 0.71 | | 2 | | |
| Males | A4 | 1.1E−06 | 1.91 | 710 | 0.17 | 614 | 0.09 | 0.79 | | 2 | | |
| stroke | B4 | 3.1E−05 | 2.11 | 574 | 0.11 | 612 | 0.06 | 0.72 | | 2 | | |
| Females | A4 | 9.9E−03 | 1.49 | 528 | 0.13 | 614 | 0.10 | 0.74 | | 2 | | |
| stroke | B4 | 6.3E−02 | 1.47 | 426 | 0.08 | 612 | 0.06 | 0.70 | | 2 | | |
| All stroke | | 8.4E−05 | 1.65 | 1054 | 0.15 | 614 | 0.09 | 0.78 | | 2 | | |
| excluding MI | | | | | | | | | | | | |
| Males stroke | | 6.4E−05 | 1.78 | 573 | 0.16 | 614 | 0.09 | 0.75 | | 2 | | |
| excluding MI | | | | | | | | | | | | |
| Females | | 1.2E−02 | 1.49 | 481 | 0.14 | 614 | 0.10 | 0.72 | | 2 | | |
| stroke | | | | | | | | | | | | |
| excluding MI | | | | | | | | | | | | |
| Cardioembolic | | 6.6E−04 | 1.87 | 248 | 0.16 | 614 | 0.10 | 0.74 | | 2 | | |
| stroke | | | | | | | | | | | | |
| Cardioembolic | | 3.8E−02 | 1.56 | 191 | 0.14 | 614 | 0.10 | 0.70 | | 2 | | |
| stroke | | | | | | | | | | | | |
| excluding MI | | | | | | | | | | | | |
| Large vessel | | 8.0E−02 | 1.47 | 150 | 0.13 | 614 | 0.09 | 0.83 | | 2 | | |
| stroke | | | | | | | | | | | | |
| Large vessel | | 2.9E−01 | 1.31 | 114 | 0.12 | 614 | 0.09 | 0.80 | | 2 | | |
| stroke | | | | | | | | | | | | |
| excluding MI | | | | | | | | | | | | |
| Small vessel | | 7.2E−04 | 2.05 | 166 | 0.18 | 614 | 0.09 | 0.71 | | 2 | | |
| stroke | | | | | | | | | | | | |
| Small vessel | | 1.0E−04 | 2.31 | 152 | 0.20 | 614 | 0.10 | 0.71 | | 2 | | |
| stroke excluding MI | | | | | | | | | | | | |
| Hemorrhagic | | 4.4E−02 | 1.73 | 97 | 0.15 | 614 | 0.09 | 0.72 | | 2 | | |
| stroke | | | | | | | | | | | | |
| Hemorrhagic | | 3.9E−02 | 1.78 | 92 | 0.16 | 614 | 0.09 | 0.71 | | 2 | | |
| stroke excluding MI | | | | | | | | | | | | |
| Unknown | | 1.3E−04 | 1.88 | 335 | 0.16 | 614 | 0.09 | 0.75 | | 2 | | |
| cause stroke | | | | | | | | | | | | |
| Unknown cause | | 6.5E−04 | 1.82 | 297 | 0.16 | 614 | 0.09 | 0.72 | | 2 | | |
| stroke excluding MI | | | | | | | | | | | | |
| MI and stroke | | | | | | | | | | | | |
| together | | | | | | | | | | | | |
| All patients | Best haplo A4 | 4.1E−07 | 1.75 | 2659 | 0.15 | 614 | 0.09 | 0.82 | | 2 | | |
| | B4 | 4.1E−05 | 1.85 | 2205 | 0.10 | 612 | 0.06 | 0.70 | | 2 | | |
| Males | A4 | 1.4E−08 | 1.93 | 1437 | 0.17 | 614 | 0.09 | 0.82 | | 2 | | |
| | B4 | 2.0E−06 | 2.11 | 1290 | 0.11 | 612 | 0.06 | 0.70 | | 2 | | |
| Females | A4 | 3.6E−03 | 1.47 | 1024 | 0.13 | 614 | 0.09 | 0.77 | | 2 | | |
| | B4 | 2.8E−02 | 1.48 | 915 | 0.08 | 612 | 0.06 | 0.66 | | 2 | | |
| Patients with | A4 | 6.1E−05 | 2.10 | 184 | 0.18 | 614 | 0.09 | 0.86 | | 2 | | |
| both MI and stroke | | | | | | | | | | | | |
| Replication In | | | | | | | | | | | | |
| PAOD | | | | | | | | | | | | |
| All PAOD | | 3.6E−02 | 1.31 | 920 | 0.12 | 614 | 0.10 | 0.84 | | 2 | | |
| patients | | | | | | | | | | | | |
| Males PAOD | | 1.8E−02 | 1.40 | 580 | 0.13 | 614 | 0.10 | 0.84 | | 2 | | |
| Females PAOD | | 3.7E−01 | 1.17 | 340 | 0.11 | 614 | 0.10 | 0.83 | | 2 | | |
| All PAOD | | 1.1E−01 | 1.24 | 750 | 0.12 | 614 | 0.10 | 0.83 | | 2 | | |
| excluding MI | | | | | | | | | | | | |
| Males PAOD | | 8.3E−02 | 1.30 | 461 | 0.12 | 614 | 0.10 | 0.83 | | 2 | | |
| excluding MI | | | | | | | | | | | | |
| Males PAOD | | 8.7E−02 | 1.32 | 388 | 0.12 | 614 | 0.10 | 0.83 | | 2 | | |
| excluding MI | | | | | | | | | | | | |
| and stroke | | | | | | | | | | | | |

| | | DG00AAHII | DG00AAHID | SG13S26 | B_SNP_310657 | SG13S30 | SG13S32 | SG13S41 | SG13S42 |
|---|---|---|---|---|---|---|---|---|---|
| MI haplotypes | | | | | | | | | |
| All MI | A4 | | 3 | | 2 | | 0 | | |
| patients | B4 | 2 | | | | 2 | | | 0 |
| Males MI | A4 | | 3 | | 2 | | 0 | | |
| | B4 | 2 | | | | 2 | | | 0 |
| Females MI | A4 | | 3 | | 2 | | 0 | | |
| | B4 | 2 | | | | 2 | | | 0 |
| Replication in stroke | | | | | | | | | |
| All stroke | A4 | | 3 | | 2 | | 0 | | |
| patients | B4 | 2 | | | | 2 | | | 0 |
| Males | A4 | | 3 | | 2 | | 0 | | |
| stroke | B4 | 2 | | | | 2 | | | 0 |
| Females | A4 | | 3 | | 2 | | 0 | | |
| stroke | B4 | 2 | | | | 2 | | | 0 |
| All stroke | | | 3 | | 2 | | 0 | | |
| excluding MI | | | | | | | | | |
| Males stroke | | | 3 | | 2 | | 0 | | |
| excluding MI | | | | | | | | | |
| Females | | | 3 | | 2 | | 0 | | |
| stroke | | | | | | | | | |
| excluding MI | | | | | | | | | |
| Cardioembolic | | | 3 | | 2 | | 0 | | |
| stroke | | | | | | | | | |
| Cardioembolic | | | 3 | | 2 | | 0 | | |
| stroke | | | | | | | | | |
| excluding MI | | | | | | | | | |
| Large vessel | | | 3 | | 2 | | 0 | | |
| stroke | | | | | | | | | |
| Large vessel | | | 3 | | 2 | | 0 | | |
| stroke | | | | | | | | | |
| excluding MI | | | | | | | | | |
| Small vessel | | | 3 | | 2 | | 0 | | |
| stroke | | | | | | | | | |
| Small vessel | | | 3 | | 2 | | 0 | | |
| stroke | | | | | | | | | |
| excluding MI | | | | | | | | | |
| Hemorrhagic | | | 3 | | 2 | | 0 | | |
| stroke | | | | | | | | | |
| Hemorrhagic | | | 3 | | 2 | | 0 | | |
| stroke | | | | | | | | | |
| excluding MI | | | | | | | | | |
| Unknown | | | 3 | | 2 | | 0 | | |
| cause stroke | | | | | | | | | |
| Unknown cause | | | 3 | | 2 | | 0 | | |
| stroke excluding MI | | | | | | | | | |
| MI and stroke together | | | | | | | | | |
| All patients | Best haplo A4 | | 3 | | 2 | | 0 | | |
| | B4 | 2 | | | | 2 | | | 0 |
| Males | A4 | | 3 | | 2 | | 0 | | |
| | B4 | 2 | | | | 2 | | | 0 |
| Females | A4 | | 3 | | 2 | | 0 | | |
| | B4 | 2 | | | | 2 | | | 0 |
| Patients with | A4 | | 3 | | 2 | | 0 | | |
| both MI and stroke | | | | | | | | | |
| Replication In PAOD | | | | | | | | | |
| All PAOD | | | 3 | | 2 | | 0 | | |
| patients | | | | | | | | | |
| Males PAOD | | | 3 | | 2 | | 0 | | |
| Females PAOD | | | 3 | | 2 | | 0 | | |
| All PAOD | | | 3 | | 2 | | 0 | | |
| excluding MI | | | | | | | | | |
| Males PAOD | | | 3 | | 2 | | 0 | | |
| excluding MI | | | | | | | | | |
| Males PAOD | | | 3 | | 2 | | 0 | | |
| excluding MI and | | | | | | | | | |
| stroke | | | | | | | | | |

### SUMMARY

In summary, it has been found that: MI correlates with genetic variation at FLAP; MI correlates with high expression promoter polymorphism at 5-LO; patients with female MI at-risk FLAP haplotypes have higher levels of serum LTE4; LTE4 levels correlate with CRP levels in serum; and patients with MI at-risk FLAP haplotypes have elevated CRP. Taken together, these results show that increased leukotriene synthesis is a risk factor for MI, especially but not only in females, and that this risk is driven in part by variants in FLAP and 5-LO genes and are captured in part by measurement of levels of serum LTE4 and CRP. Furthermore, the SNP haplotype in the FLAP gene that confers risk to MI also confers risk of stroke and/or PAOD.

### EXAMPLE 9

### Additional Correlation Between Flap Gene and MI, Stroke and PAOD

A genome wide scan of 296 multiplex Icelandic families with 713 MI patients was performed. This geneome-wide scan involves more MI phenotypes than described in Example 1. The cohort is a subset of the study population described in Example 1; in this cohort, related individuals were assessed. Through the suggestive linkage to a locus on chromosome 13q12-13 for female MI patients and early onset MI patients, and a new microsatellite marker association analysis (including more microsatellite markers than described in Example 1), the gene encoding the 5-lipoxygenase activating protein (FLAP) was again identified, and a 4-SNP haplotype within the gene was determined to confer a near 2-fold risk of MI and stroke. Male patients showed strongest association to the at-risk haplotype. Independent confirmation of FLAP association to MI was obtained in a British cohort of patients with sporadic MI. These findings support FLAP as the first specific gene isolated that confers substantial risk of the complex traits of MI and stroke.

Methods

Study Population

The study population was the same as used in Example 1.

Genotypes from 713 MI patients and 1741 of their first-degree relatives were used in the linkage analysis. For the microsatellite association study of the MI locus, 802 unrelated MI patients (n=233 females, n=624 males and n=302 early onset) and 837 population-based controls were used. For the SNP association study in and around the FLAP gene 779 unrelated MI patients were genotyped (n=293 females, n=486 males and n=358 early onset). The control group for the SNP association study was population based and comprised of 628 unrelated males and females in the age range of 30-85 years whose medical history was unknown. The stroke and PAOD cohorts used in this study have previously been described (Gretarsdottir, S. et al. Nat Genet 35, 131-8 (2003); Gretarsdottir, S. et al., Am J Hum Genet 70, 593-603 (2002); Gudmundsson, G. et al., Am J Hum Genet 70, 586-92 (2002)). For the stroke linkage analysis, genotypes from 342 male patients with ischemic stroke or TIA that were linked to at least one other male patient within and including 6 meioses in 164 families were used. For the association studies 702 patients with all forms of stroke (n=329 females and n=373 males) and 577 PAOD patients (n=221 females and n=356 males) were analysed. Patients with stroke or PAOD that also had MI were excluded. Controls used for the stroke and PAOD association studies were the same as used in the MI SNP association study (n=628).

The study was approved by the Data Protection Commission of Iceland and the National Bioethics Committee of Iceland. Informed consent was obtained from all study participants. Personal identifiers associated with medical information and blood samples were encrypted with a third party encryption system as previously described (Gulcher, J. R., Kristjansson; K., Gudbjartsson, H. & Stefansson, K., Eur J Hum Genet 8, 739-42 (2000)).

Statistical Analysis

A genome-wide scan was performed as previously described (Gretarsdottir, S. et al. Am J Hum Genet 70, 593-603 (2002)), using a set of 1000 microsatellite markers. Multipoint, affected-only allele-sharing methods (Kong, A. & Cox, N. J., Am J Hum Genet 61, 1179-88 (1997)) were used to assess the evidence for linkage. All results were obtained using the program Allegro (Gudbjartsson, D. F., Jonasson, K., Frigge, M. L. & Kong, A. Allegro, Nat Genet 25, 12-3 (2000)) and the deCODE genetic map (Kong, A. et al., Nat Genet 31, 241-7 (2002)). The Sₚₐᵢᵣₛ scoring function (Whittemore, A. S. & Halpern, J., Biometrics 50, 118-27 (1994); Kruglyak, L., Daly, M. J., Reeve-Daly, M. P. & Lander, E. S., Am J Hum Genet 58, 1347-63 (1996)) was used, as was the exponential allele-sharing model (Kong, A. & Cox, N. J. Am J Hum Genet 61, 1179-88 (1997)) to generate the relevant 1-df (degree of freedom) statistics. When combining the family scores to obtain an overall score, a weighting scheme was used that is halfway on a log scale between weighting each affected pair equally and weighting each family equally. In the analysis, all genotyped individuals who are not affected are treated as “unknown”. Because of concern with small sample behaviour, corresponding P values were usually computede in two different ways for comparison, and the less significant one was reported. The first P value is computed based on large sample theory; Zᵢᵣ=√(2 logₑ(10) LOD) and is distributed approximately as a standard normal distribution under the null hypothesis of no linkage (Kong, A. & Cox, N. J. Am J Hum Genet 61, 1179-88 (1997)). A second P value is computed by comparing the observed LOD score to its complete data sampling distribution under the null hypothesis (Gudbjartsson, D. F., Jonasson, K., Frigge, M. L. & Kong, A. Allegro, Nat Genet 25, 12-3 (2000)). When a data set consists of more than a handful of families, these two P values tend to be very similar. The information measure that was used (Nicolae, D. University of Chicago (1999)), and is implemented in Allegro, is closely related to a classical measure of information (Dempster, A., Laird, N M, Rubin, D B., J R Stat Soc B 39, 1-38 (1977) and has a property that is between 0, if the marker genotypes are completely uninformative, and 1, if the genotypes determine the exact amount of allele sharing by descent among the affected relatives.

For single-marker association studies, Fisher's exact test was used to calculate two-sided P values for each allele. All P values were unadjusted for multiple comparisons unless specifically indicated. Allelic rather than carrier frequencies were presented for microsatellites, SNPs and haplotypes. To minimize any bias due to the relatedness of the patients that were recruited as families for the linkage analysis first and second-degree relatives were eliminated from the patient list. For the haplotype analysis, the program NEMO was used (Gretarsdottir, S. et al., Nat Genet 35, 131-8 (2003)), which handles missing genotypes and uncertainty with phase through a likelihood procedure, using the expectation-maximization algorithm as a lo computational tool to estimate haplotype frequencies. Under the null hypothesis, the affected individuals and controls are assumed to have identical haplotype frequencies. Under the alternative hypotheses, the candidate at-risk haplotype is allowed to have a higher frequency in the affected individuals than in controls, while the ratios of frequencies of all other haplotypes are assumed to be the same in both groups. Likelihoods are maximized separately under both hypotheses, and a corresponding 1-df likelihood ratio statistic used to evaluate statistical significance (id). Even though searches were only performed for haplotypes that increase the risk, all reported P values are two-sided unless otherwise stated. To assess the significance of the haplotype association corrected for multiple testing, a randomisation test was carried out using the same genotype data. The cohorts of affected individuals and controls were randomized, and the analysis was repeated. This procedure was repeated up to 1,000 times and the P value presented is the fraction of replications that produced a P value for a haplotype tested that is lower than or equal to the P value observed using the original patient and control cohorts.

For both single-marker and haplotype analysis, relative risk (RR) and population attributable risk was calculated assuming a multiplicative model (Terwilliger, J. D. & Ott, J. A., Hum Hered 42, 337-46 (1992); Falk, C. T. & Rubinstein, P., Ann Hum Genet 51 (Pt 3), 227-33 (1987)) in which the risk of the two alleles of haplotypes a person carries multiply. We calculated LD between pairs of SNPs using the standard definition of D′ (Lewontin, R. C., Genetics 50, 757-82 (1964)) and R² (Hill, W. G. & Robertson, A., Genetics 60, 615-28 (1968)). Using NEMO, frequencies of the two marker allele combinations are estimated by maximum likelihood, and deviation from linkage equilibrium is evaluated by a likelihood ratio test. When plotting all SNP combinations to elucidate the LD structure in a particular region, D′ was plotted in the upper left corner and the P value in the lower right corner. In the LD plots presented, the markers are plotted equidistantly rather than according to their physical positions.

Identification of DNA Polymorphisms.

New polymorphic repeats (i.e. dinucleotide or trinucleotide repeats) were identified with the Sputnik program (http://abajian.net/sputnik/index.html). The lower allele of the CEPH sample 1347-02 (CEPH genomics repository) was subtracted from the alleles of the microsatellites and used as a reference. Single nucleotide polymorphisms in the gene were detected by PCR sequencing exonic and intronic regions from patients and controls. Public single nucleotide polymorphisms were obtained from the NCBI SNP database (http://www.ncbi.nlm.nih.gov/SNP/). SNPs were genotyped using a method for detecting SNPs with fluorescent polarization template-directed dye-terminator incorporation (SNP-FP-TDI assay) (Chen, X., Zehnbauer, B., Gnirke, A. & Kwok, P. Y., Proc Natl Acad Sci U S A 94, 10756-61. (1997)) and TaqMan assays (Applied Biosystems).

British Study Population

The method of recruitment of 3 separate cohorts of British subjects has been described previously (Steeds, R., Adams, M., Smith, P., Channer, K. & Samani, N. J., Thromb Haemost 79, 980-4 (1998); Brouilette, S., Singh, R. K., Thompson, J. R., Goodall, A. H. & Samani, N. J., Arterioscler Thromb Vasc Biol 23, 842-6 (2003)). In brief, in the first two cohorts a total of 547 patients included those who were admitted to the coronary care units (CCU) of the Leicester Royal Infirmary, Leicester (July 1993-April 1994) and the Royal Hallamshire Hospital, Sheffield (November 1995-March 1997) and satisfied the World Health Organisation criteria for acute MI in terms of symptoms, elevations in cardiac enzymes or electrocardiographic changes (Nomenclature and criteria for diagnosis of ischemic heart disease. Report of the Joint International Society and Federation of Cardiology/World Health Organization task force on standardization of clinical nomenclature. Circulation 59, 607-9 (1979)). A total of 530 control subjects were recruited in each hospital from adult visitors to patients with non-cardiovascular disease on general medical, surgical, orthopaedic and obstetric wards to provide subjects likely to be representative of the source population from which the subjects originated. Subjects who reported a history of coronary heart disease were excluded.

In the third cohort, 203 subjects were recruited retrospectively from the registries of 3 coronary care units in Leicester. All had suffered an MI according to WHO criteria before the age of 50 years. At the time of participation, patients were at least 3 months from the acute event. The control cohort comprised 180 subjects with no personal or family history of premature coronary heart disease, matched for age, sex, and current smoking status with the cases. Control subjects were recruited from 3 primary care practices located within the same geographical area. In all cohorts subjects were white of Northern European origin.

Results

Linkage Analysis

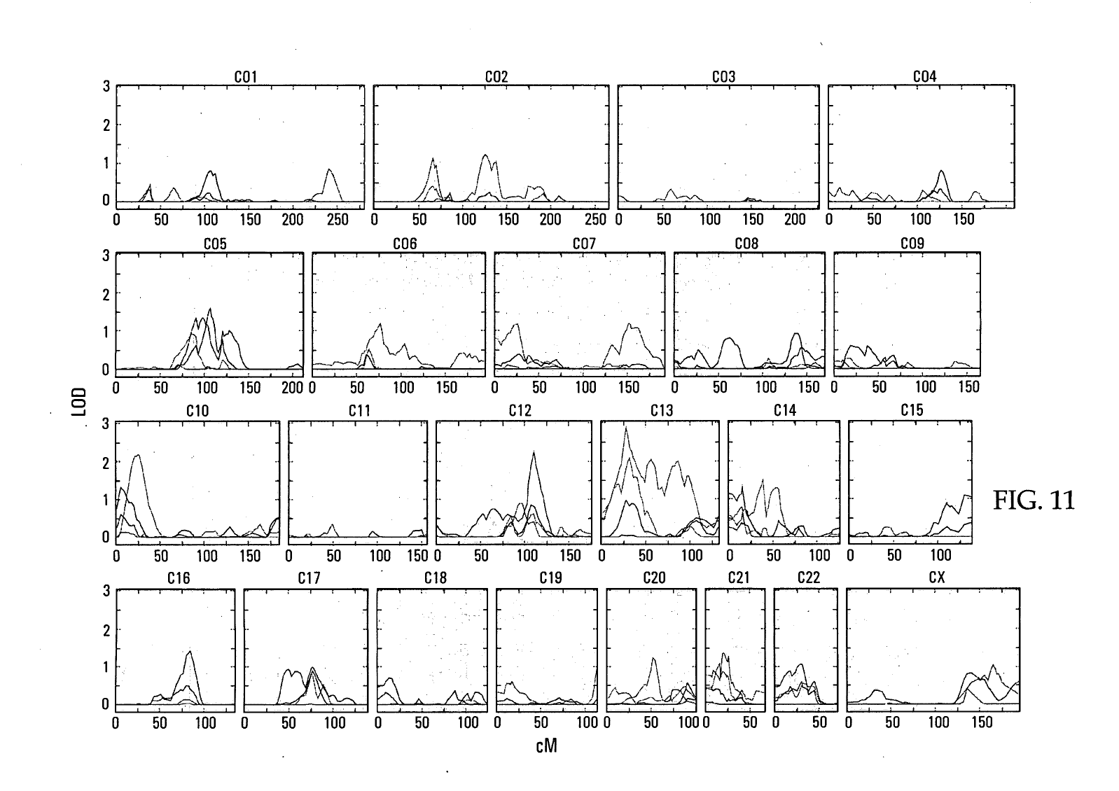

A genome wide scan was performed in search of MI susceptibility genes using a framework set of around 1000 microsatellite markers. The initial linkage analysis included 713 MI patients who fulfilled the WHO MONICA research criteria (The World Health Organization MONICA Project, WHO MONICA Project Principal Investigators, J Clin Epidemiol 41, 105-14 (1988)) and were clustered in 296 extended families. The linkage analysis was also repeated for early onset, male and female patients separately. Description of the number of patients and families in each analysis are provided in Table 16, and the corresponding allele sharing LOD scores are shown in FIG. 11.

TABLE 16
Number of patients that cluster into families and the
corresponding number of families used
in the linkage analysis

| | Number of | Number of | Number of | Genotyped |
|---|---|---|---|---|
| Phenotype | patients | families | pairs | relatives^{a} |
| All MI patients | 713 | 296 | 863 | 1741 |
| Males | 575 | 248 | 724 | 1385 |
| Females | 140 | 56 | 108 | 366 |
| Early onset | 194 | 93 | 156 | 739 |

^{a}Genotyped relatives were used to increase the information on IBD sharing among the patients in the linkage analysis

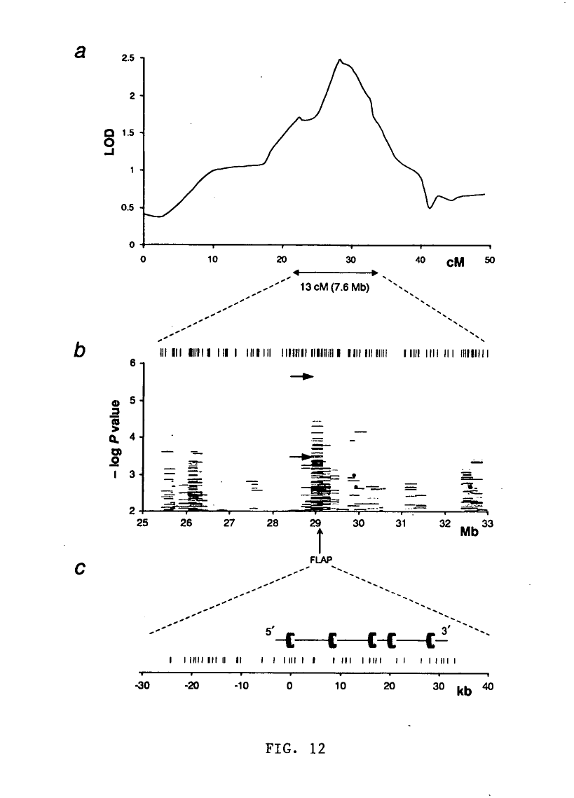

None of these analyses yielded a locus of genome-wide significance. However, the most promising LOD score (LOD=2.86) was observed on chromosome 13q12 for female MI patients at the peak marker D13S289 (FIG. 11). This locus also had the most promising LOD score (LOD=2.03) for patients with early onset MI. After increasing the information on identity-by-descent sharing to over 90% by typing 14 additional microsatellite markers in a 30 centiMorgan (cM) region around D13S289, the LOD score from the female analysis dropped to 2.48 (P value=0.00036), while the highest LOD score remained at D13S289 (FIG. 12(a)). In addition, in an independent linkage study of male patients with ischemic stroke or transient ischemic attack we observed linkage to the same locus with a LOD score of 1.51 at the same peak marker (FIG. 13), further suggesting that a cardiovascular susceptibility factor might reside at this locus.

Microsatellite Association Study

The 7.6 Mb region that corresponds to a drop of one in LOD score in the female MI analysis, contains 40 known genes (Table 17).

TABLE 17
Genes residing within the one LOD drop region of
the chromosome 13q12 linkage
peak.

| LL_Symbol | LL_gene_name |
|---|---|
| USP12L1 | ubiquitin specific protease 12 like 1 |
| RPL21 | ribosomal protein L21 |
| GTF3A | general transcription factor IIIA |
| MTIF3 | mitochondrial translational initiation factor 3 |
| PDZRN1 | PDZ domain containing ring finger 1 |
| MGC9850 | hypothetical protein MGC9850 |
| POLR1D | polymerase (RNA) I polypeptide D, 16 kDa |
| GSH1 | GS homeobox 1 |
| IPF1 | insulin promoter factor 1, homeodomain transcription factor |
| CDX2 | caudal type homeo box transcription factor 2 |
| FLT3 | fms-related tyrosine kinase 3 |
| LOC255967 | hypothetical protein LOC255967 |
| FLT1 | fms-related tyrosine kinase 1 (vascular endothelial |
| | growth factor/vascular permeability factor receptor) |
| C13orf12 | chromosome 13 open reading frame 12 |
| LOC283537 | hypothetical protein LOC283537 |
| KIAA0774 | KIAA0774 protein |
| SLC7A1 | solute carrier family 7 (cationic amino acid transporter, |
| | y+ system), member 1 |
| UBL3 | ubiquitin-like 3 |
| MGC2599 | hypothetical protein MGC2599 similar to katanin |
| | p60 subunit A 1 2599 |
| HMGB1 | high-mobility group box 1 |
| D13S106E | highly charged protein |
| ALOX5AP | arachidonate 5-lipoxygenase-activating protein |
| FLJ14834 | hypothetical protein FLJ14834 |
| MGC40178 | hypothetical protein MGC40178 |
| HSPH1 | heat shock 105 kDa/110 kDa protein 1 |
| B3GTL | beta 3-glycosyltransferase-like |
| GREAT | similar to G protein coupled receptor affecting |
| | testicular descent (H. sapiens) |
| LOC196549 | similar to hypothetical protein FLJ20897 |
| 13CDNA73 | hypothetical protein CG003 |
| BRCA2 | breast cancer 2, early onset |
| CG018 | hypothetical gene CG018 |
| PRO0297 | PRO0297 protein |
| LOC88523 | CG016 |
| CG012 | hypothetical gene CG012 |
| CG030 | hypothetical gene CG030 |
| CG005 | hypothetical protein from BCRA2 region |
| APRIN | androgen-induced proliferation inhibitor |
| KL | Klotho |
| STARD13 | START domain containing 13 |
| RFC3 | replication factor C (activator 1) 3, 38 kDa |

To determine which gene in this region most likely contributes to MI 120 microsatellite markers were typed within this region, and a case-control association study was performed using 802 unrelated MI patients and 837 population-based controls. The association study was also repeated for each of the three phenotypes that were used in the linkage study, i.e. early onset, male and female MI patients. In addition to testing each marker individually, haplotypes constructed out of those markers for association were also tested. To limit the number of haplotypes tested, only haplotypes that were in excess in the patient cohorts and that spanned less than 300 kb were assessed (see Methods).

As shown in FIG. 12(b), the haplotype that showed association to all MI with the lowest P value (0.00009) covered a region that contains 2 known genes, including the gene encoding arachidonate 5-lipoxygenase-activating protein (FLAP) and a gene with an unknown function called highly charged protein. However, the haplotype association to female MI in this region was less significant (P value=0.005) than for all MI patients and to our surprise, the most significant haplotype association was observed for male MI patients (P value=0.000002). This male MI haplotype was the only haplotype that remained significant after adjusting for all haplotypes tested.

In view of the association results described above, FLAP was an attractive candidate and therefore efforts were focused on this gene.

Screening for Polymorphisms in FLAP and Linkage Disequilibrium Mapping

To determine whether variations within the FLAP gene significantly associate with MI and to search for causal variations, the FLAP gene was sequenced in 93 patients and 93 controls. The sequenced region covers 60 kb containing the FLAP gene, including the 5 known exons and introns and the 26 kb region 5′ to the first exon and 7 kb region 3′ to the fifth exon. In all, 144 SNPs were identified, of those 96 were excluded from further analysis either because of low minor allele frequency or they were completely correlated with other SNPs and thus redundant. FIG. 12(c) shows the distribution of the 48 SNPs, used for genotyping, relative to exons, introns and the 5′and 3′flanking regions of the FLAP gene. Only one SNP was identified within a coding sequence (exon 2). This SNP did not lead to amino acid substitution. The locations of these SNPs in the NCBI human genome assembly, build 34, are listed in Table 18.

TABLE 18
Locations of all genotyped SNPs in NCBI build
34 of the human genome assembly.

| SNP name | Build34 start |
|---|---|
| SG13S381 | 29083350 |
| SG13S366 | 29083518 |
| SG13S1 | 29086224 |
| SG13S2 | 29087473 |
| SG13S367 | 29088090 |
| SG13S10 | 29088473 |
| SG13S3 | 29089044 |
| SG13S368 | 29089886 |
| SG13S4 | 29090997 |
| SG13S5 | 29091307 |
| SG13S90 | 29091780 |
| SG13S6 | 29092536 |
| SG13S371 | 29093964 |
| SG13S372 | 29094259 |
| SG13S373 | 29096688 |
| SG13S375 | 29096874 |
| SG13S376 | 29096962 |
| SG13S25 | 29097553 |
| SG13S377 | 29101965 |
| SG13S100 | 29104271 |
| SG13S95 | 29106329 |
| SG13S191 | 29107830 |
| SG13S106 | 29108579 |
| SG13S114 | 29110096 |
| SG13S121 | 29112174 |
| SG13S122 | 29112264 |
| SG13S43 | 29112455 |
| SG13S192 | 29116308 |
| SG13S88 | 29116401 |
| SG13S137 | 29118118 |
| SG13S86 | 29118815 |
| SG13S87 | 29118873 |
| SG13S39 | 29119740 |
| SG13S26 | 29122253 |
| SG13S27 | 29122283 |
| SG13S29 | 29123643 |
| SG13S89 | 29124441 |
| SG13S96 | 29124906 |
| SG13S30 | 29125840 |
| SG13S97 | 29129139 |
| SG13S32 | 29130547 |
| SG13S41 | 29134045 |
| SG13S42 | 29135877 |
| SG13S34 | 29137100 |
| SG13S35 | 29138117 |
| SG13S181 | 29138633 |
| SG13S184 | 29139435 |
| SG13S188 | 29140805 |

In addition to the SNPs, a polymorphism consisting of a monopolymer A repeat that has been described in the FLAP promoter region was typed (Koshino, T. et al., Mol Cell Biol Res Commun 2, 32-5 (1999)).

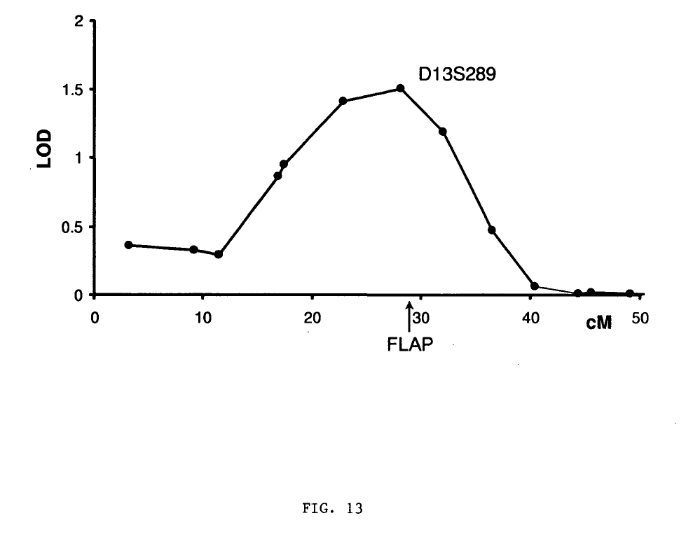

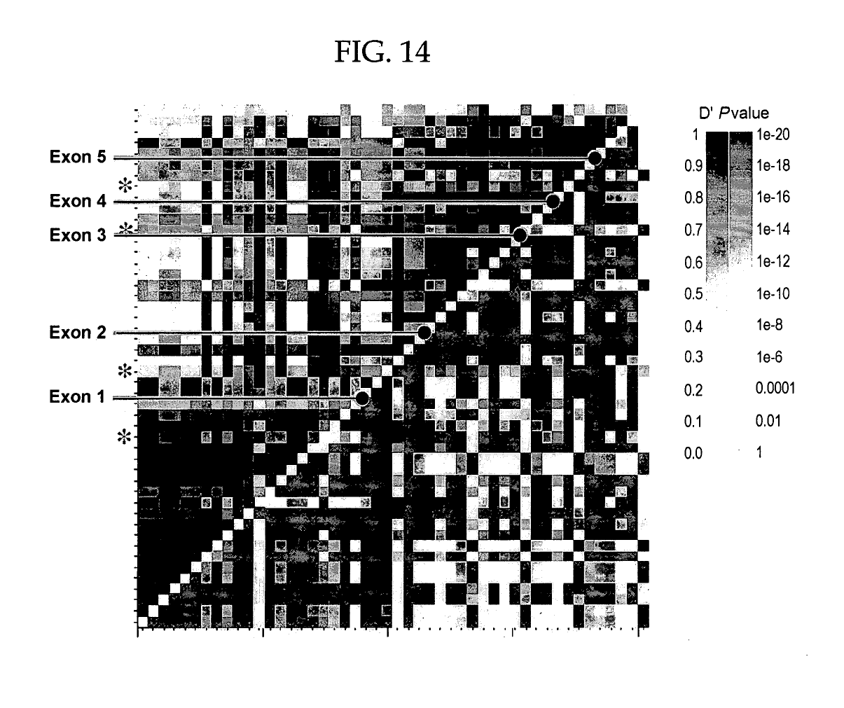

The linkage disequilibrium (LD) block structure defined by the 48 SNPs that were selected for further genotyping is shown in FIG. 14. A strong LD was detected across the FLAP region, although it appears that at least one recombination may have occurred dividing the region into two strongly correlated LD blocks.

Haplotype Association to MI

To perform a case-control association study the 48 selected SNPs and the monopolymer A repeat marker were genotyped in a set of 779 unrelated MI patients and 628 population-based controls. Each of the 49 markers were tested individually for association to the disease. Three SNPs, one located 3 kb upstream of the first exon and the other two 1 and 3 kb downstream of the first exon, showed nominally significant association to MI (Table 19).

TABLE 19
SNP allelic association in the MI cohort

| Phe- | | Al- | | | # | % | # | % |
|---|---|---|---|---|---|---|---|---|
| notype | Marker | lele | P value | RR | Pat. | Pat. | Ctrl | Ctrl |
| All | SG13S106 | G | 0.0044 | 1.29 | 681 | 72.0 | 530 | 66.6 |
| patients | SG13S100 | A | 0.020 | 1.29 | 388 | 69.6 | 377 | 63.9 |
| | SG13S114 | T | 0.021 | 1.21 | 764 | 70.0 | 602 | 65.8 |
| Males | SG13S106 | G | 0.0037 | 1.35 | 422 | 72.9 | 530 | 66.6 |
| | SG13S100 | A | 0.0099 | 1.36 | 292 | 70.7 | 377 | 63.9 |
| | SG13S114 | T | 0.026 | 1.24 | 477 | 70.4 | 602 | 65.8 |
| Early | SG13S100 | A | 0.0440 | 1.43 | 99 | 71.7 | 377 | 63.9 |
| onset | | | | | | | | |

Nominally significant SNP association with corresponding number of patients (# Pat.) and controls (#Ctrl).
RR refers to relative risk.

However, after adjusting for the number of markers tested, these results were not significant. A search was then conducted for haplotypes that show association to the disease using the same cohorts. For computational reasons, the search was limited to haplotype combinations constructed out of two, three or four SNPs and only haplotypes that were in excess in the patients were tested. The resulting P values were adjusted for all the haplotypes we tested by randomizing the patients and controls (see Methods).

Several haplotypes were found that were significantly associated to the disease with an adjusted P value less that 0.05 (Table 20).

TABLE 20
SNP haplotypes that significantly associate with Icelandic MI patients

| SG13S4 | SG13S6 | SG13S372 | SG13S25 | SG13S377 | SG13S100 | SG13S95 | SG13S114 | SG13S192 | SG13S137 | SG13S86 | SG13S87 | SG13S39 |
|---|---|---|---|---|---|---|---|---|---|---|---|---|
| | | | G | | | | T | | | | | |
| | | | G | | | | T | | | | A | |
| | | | G | | | | T | | | | | |
| | | | G | | A | | | | | | A | |
| | | | G | | | T | T | | | | | |
| | | | G | | | | T | | | G | | |
| | | | G | | A | | | | | | | |
| | | | G | | A | | | | | | | |
| | | | G | | | | T | | | | | |
| | | | G | | | | T | | | | | |
| | G | | | | | T | T | | | | | |
| | | | G | | A | | | | | G | | |
| | | | G | | | | T | A | | | | |
| | | | G | | | | T | | | | | |
| | G | | | | | | T | | | | | |
| | G | | G | | | | T | | | | | |
| | G | | | | A | | | | | | | |
| | | | G | | A | | | | | | | |
| | G | | | | A | | | | | | A | |
| | | | G | | A | | | | | | | |
| | G | | | | | | T | | | | A | |
| | | | G | | A | | | A | | | | |
| | | | G | | | | T | | | | | G |
| | G | | | | A | | | | | | | |
| | G | | | | | | T | | | | | |
| | | | G | | A | | | | | | | |
| | G | | | | | | T | | | | | |
| | G | | G | | A | | | | | | | |
| | | | G | | A | | | A | | | | |
| | | | G | | | | T | A | | | | |
| | | | G | | A | | | A | | | | |
| | | | G | | | | T | | C | | | |
| | | | G | | | | T | | | | | |
| | | | G | | | | T | | C | | | |
| | | | G | G | A | | | | | | | |
| | | | G | | | | T | | | | | |
| | | | G | | | | T | | | G | | |
| | | | G | | | A | | | | | | |
| | | | G | | A | | | | | | | G |
| C | G | | | | A | | | | | | | |
| | G | | | | | | T | A | | | | |
| | G | | | | A | | | | | | | |
| | G | | | | | | T | | | | | |
| | | | G | | | | T | | | | | |
| | G | G | | | A | | | | | | | |
| | G | | G | | A | | | A | | | | |

| SG13S27 | SG13S89 | SG13S96 | SG13S32 | SG13S41 | SG13S42 | SG13S34 | SG13S188 | P value^{a} | P value^{b} | Pat.frq | Ctrl.frq | RR | D′^{c} |
|---|---|---|---|---|---|---|---|---|---|---|---|---|---|
| | G | | A | | | | | 0.0000023 | 0.005 | 0.158 | 0.095 | 1.80 | 1.00 |
| | | | A | | | | | 0.0000030 | 0.006 | 0.158 | 0.095 | 1.78 | 1.00 |
| | | | A | | | T | | 0.0000032 | 0.007 | 0.157 | 0.094 | 1.79 | 1.00 |
| | | | A | | | | | 0.0000046 | 0.012 | 0.158 | 0.083 | 2.07 | 0.89 |
| | | | A | | | | | 0.0000047 | 0.012 | 0.154 | 0.093 | 1.78 | 1.00 |
| | | | A | | | | | 0.0000055 | 0.015 | 0.147 | 0.087 | 1.81 | 1.00 |
| | | | A | | | T | | 0.0000061 | 0.017 | 0.157 | 0.083 | 2.07 | 0.89 |
| | G | | A | | | | | 0.0000063 | 0.017 | 0.157 | 0.084 | 2.04 | 0.89 |
| | | | A | | | | | 0.0000070 | 0.021 | 0.157 | 0.096 | 1.76 | 1.00 |
| | | A | A | | | | | 0.0000075 | 0.022 | 0.149 | 0.089 | 1.78 | 1.00 |
| | | | A | | | | | 0.0000083 | 0.024 | 0.208 | 0.139 | 1.62 | 0.99 |
| | | | A | | | | | 0.0000084 | 0.026 | 0.145 | 0.074 | 2.14 | 0.88 |
| | | | A | | | | | 0.0000084 | 0.026 | 0.139 | 0.082 | 1.82 | 1.00 |
| G | | | A | | | | | 0.0000091 | 0.028 | 0.156 | 0.096 | 1.75 | 1.00 |
| | | | A | | | T | | 0.0000094 | 0.028 | 0.210 | 0.141 | 1.61 | 0.99 |
| | | | A | | | | | 0.0000100 | 0.028 | 0.156 | 0.096 | 1.74 | 1.00 |
| | | | A | | | | A | 0.0000101 | 0.028 | 0.215 | 0.133 | 1.80 | 0.81 |
| | | | A | | | | | 0.0000105 | 0.028 | 0.157 | 0.084 | 2.03 | 0.89 |
| | | | A | | | | | 0.0000108 | 0.029 | 0.214 | 0.133 | 1.78 | 0.81 |
| | | A | A | | | | | 0.0000110 | 0.030 | 0.146 | 0.075 | 2.10 | 0.88 |
| | | | A | | | | | 0.0000112 | 0.030 | 0.212 | 0.144 | 1.60 | 1.00 |
| | | | | | | T | | 0.0000113 | 0.030 | 0.151 | 0.081 | 2.03 | 0.78 |
| | | | A | | | | | 0.0000118 | 0.031 | 0.156 | 0.096 | 1.73 | 1.00 |
| | | | A | | | T | | 0.0000126 | 0.034 | 0.212 | 0.131 | 1.79 | 0.79 |
| | G | | A | | | | | 0.0000129 | 0.035 | 0.211 | 0.144 | 1.59 | 1.00 |
| G | | | A | | | | | 0.0000134 | 0.035 | 0.156 | 0.084 | 2.01 | 0.89 |
| | | | A | | | | | 0.0000136 | 0.036 | 0.211 | 0.143 | 1.60 | 1.00 |
| | | | A | | | | | 0.0000137 | 0.036 | 0.156 | 0.085 | 2.00 | 0.89 |
| | | A | | | | | | 0.0000148 | 0.037 | 0.151 | 0.081 | 2.01 | 0.78 |
| | | | | | | T | | 0.0000150 | 0.037 | 0.160 | 0.099 | 1.73 | 0.87 |
| | | A | | | | | | 0.0000150 | 0.037 | 0.130 | 0.066 | 2.13 | 0.90 |
| | | | | | | T | | 0.0000154 | 0.039 | 0.152 | 0.094 | 1.73 | 0.93 |
| | | | A | | A | | | 0.0000154 | 0.040 | 0.155 | 0.097 | 1.70 | 1.00 |
| | | | A | | | | | 0.0000157 | 0.040 | 0.141 | 0.085 | 1.76 | 1.00 |
| | | | A | | | | | 0.0000158 | 0.040 | 0.152 | 0.084 | 1.94 | 0.90 |
| G | | | A | | | | | 0.0000163 | 0.040 | 0.210 | 0.143 | 1.59 | 0.99 |
| | | | A | | | | | 0.0000166 | 0.041 | 0.200 | 0.134 | 1.61 | 0.92 |
| | G | | A | | | | | 0.0000168 | 0.042 | 0.213 | 0.133 | 1.76 | 0.81 |
| | | | A | | | | | 0.0000168 | 0.042 | 0.156 | 0.084 | 2.00 | 0.89 |
| | | | A | | | | | 0.0000171 | 0.042 | 0.211 | 0.136 | 1.70 | 0.81 |
| | | | A | | | | | 0.0000183 | 0.043 | 0.192 | 0.128 | 1.62 | 0.85 |
| | | | A | | | | | 0.0000184 | 0.043 | 0.212 | 0.132 | 1.77 | 0.81 |
| | | | | A | | T | | 0.0000193 | 0.046 | 0.328 | 0.251 | 1.46 | 0.99 |
| G | | | | | | T | | 0.0000194 | 0.046 | 0.175 | 0.115 | 1.64 | 0.98 |
| | | | A | | | | | 0.0000202 | 0.048 | 0.210 | 0.136 | 1.70 | 0.81 |
| | | | | | | | | 0.0000209 | 0.049 | 0.151 | 0.082 | 2.00 | 0.76 |

^{a}Single test P values.
^{b}P values adjusted for all the SNP haplotypes tested.
^{c}Measure of correlation with Haplotype A4.

The most significant association was observed for a four SNP haplotype spanning 33 kb, including the first four exons of the gene (FIG. 12(c)), with a nominal P value of 0.0000023 and an adjusted P value of 0.005. This haplotype, labelled A4, has haplotype frequency of 15.8% (carrier frequency 30.3%) in patients versus 9.5% (carrier frequency 17.9%) in controls (Table 21).

TABLE 21
Association of the A4 haplotype to MI, Stroke and PAOD

| Phenotype (n) | Frq. Pat. | RR | PAR | P-value | P-value^{a} |
|---|---|---|---|---|---|
| MI (779) | 0.158 | 1.80 | 0.135 | 0.0000023 | 0.005 |
| Males (486) | 0.169 | 1.95 | 0.158 | 0.00000091 | ND^{b} |
| Females (293) | 0.138 | 1.53 | 0.094 | 0.0098 | ND |
| Early onset (358) | 0.138 | 1.53 | 0.094 | 0.0058 | ND |
| Stroke (702)^{c} | 0.149 | 1.67 | 0.116 | 0.000095 | ND |
| Males (373) | 0.156 | 1.76 | 0.131 | 0.00018 | ND |
| Females (329) | 0.141 | 1.55 | 0.098 | 0.0074 | ND |
| PAOD (577)^{c} | 0.122 | 1.31 | 0.056 | 0.061 | ND |
| Males (356) | 0.126 | 1.36 | 0.065 | 0.057 | ND |
| Females (221) | 0.114 | 1.22 | 0.041 | 0.31 | ND |
| ^{a}P value adjusted for the number of haplotypes tested. | | | | | |
| ^{b}Not done. | | | | | |
| ^{c}Excluding known cases of MI. | | | | | |
| Shown is the FLAP A4 haplotype and corresponding number of patients (n), haplotype frequency in patients (Frq. pat.), relative risk (RR), population attributed risk (PAR) and P values. The A4 haplotype is defined by the following SNPs: SG13S25, SG13S114, SG13S89 and SG13S32 (Table 20). | | | | | |
| # The same controls (n = 628) are used for the association analysis in MI, stroke and PAOD as well as for the male, female and early onset analysis. The A4 haplotype frequency in the control cohort is 0.095. | | | | | |

The relative risk conferred by The A4 haplotype compared to other haplotypes constructed out of the same SNPs, assuming a multiplicative model, was 1.8 and the corresponding population attributable risk (PAR) was 13.5%. As shown in Table 21, The A4 haplotype was observed in higher frequency in male patients (carrier frequency 30.9%) than in female patients (carrier frequency 25.7%). All the other haplotypes that were significantly associated with an adjusted P value less than 0.05, were highly correlated with The A4 haplotype and should be considered variants of that haplotype (Table 20).

Association of the A4 Haplotype to Stroke and Peripheral Arterial Occlusive Disease

In view of the linkage observed for stroke in male patients to the FLAP locus and since there is a high degree of co-morbidity among MI, stroke and peripheral arterial occlusive disease (PAOD), with most of these cases occurring on the basis of an atherosclerotic disease, it was determined whether The A4 haplotype also shows association to stroke and/or PAOD and typed the SNPs defining The A4 haplotype on these patient cohorts. First and second degree relatives and all known cases of MI were removed, and 702 stroke patients and 577 PAOD patients were tested for association. The results are also listed in Table 21, above. A significant association of The A4 haplotype to stroke was observed, with a relative risk of 1.67 (P value=0.000095). In addition, it was determined whether The A4 haplotype was primarily associated with a particular sub-phenotype of stroke, and found that both ischemic and hemorrhagic stroke were significantly associated with The A4 haplotype (Table 22).

TABLE 22
Association of The A4 haplotype to subgroups of stroke

| Phenotype (n) | Pat. Frq. | RR | PAR | P-value |
|---|---|---|---|---|
| Stroke^{a} (702) | 0.149 | 1.67 | 0.116 | 0.000095 |
| Ischemic (484) | 0.148 | 1.65 | 0.113 | 0.00053 |
| TIA (148) | 0.137 | 1.51 | 0.090 | 0.058 |
| Hemorrhagic (68) | 0.167 | 1.91 | 0.153 | 0.024 |

^{a}Excluding known cases of MI.

Finally, although The A4 haplotype was more frequent in the PAOD cohort than in the population controls (Table 21), this was not significant. It should be noted that similar to the stronger association of The A4 haplotype to male MI compared to female MI, it also shows stronger association to male stroke and PAOD (Table 15).

Haplotype Association to FLAP in a British Cohort

In an independent study, it was determined whether variants in the FLAP gene also have impact on risk of MI in a population outside Iceland. The four SNPs, defining The A4 haplotype, were typed in a cohort of 750 patients from the United Kingdom who had sporadic MI, and in 728 British population controls. The patients and controls come from 3 separate study cohorts recruited in Leicester and Sheffield. No significant differences were found in the frequency of the haplotype between patients and controls (16.9% versus 15.3%, respectively). However, when we typed additional 9 SNPs, distributed across the FLAP gene, in the British cohort and searched for other haplotypes that might be associated with MI, two SNPs showed association to MI with a nominally significant P value (data not shown). Moreover, three and four SNP haplotype combinations increased the risk of MI in the British cohort further and the most significant association was observed for a four SNP haplotype with a nominal P value=0.00037 (Table 23).

TABLE 23
Association of the HapB haplotype to British MI patients

| Phenotype (n) | Frq. Pat. | RR | PAR | P-value | P-value^{a} |
|---|---|---|---|---|---|
| MI (750) | 0.075 | 1.95 | 0.072 | 0.00037 | 0.046 |
| Males (546) | 0.075 | 1.97 | 0.072 | 0.00093 | ND |
| Females (204) | 0.073 | 1.90 | 0.068 | 0.021 | ND |
| ^{a}P value adjusted for the number of haplotypes tested using 1,000 randomization tests. | | | | | |
| Shown are the results for HapB that shows the strongest association in British MI cohort. HapB is defined by the following SNPs: SG13S377, SG13S114, SG13S41 and SG13S35 (that have the following alleles A, A, A and G, respectively. In all three phenotypes shown the same set of n = 728 British controls is used and the frequency of HapB in the control cohort is 0.040. | | | | | |
| # Number of patients (n), haplotype frequency in patients (Frq. pat.), relative risk (RR) and population attributed risk (PAR). | | | | | |

This was called haplotype HapB. The haplotype frequency of HapB is 7.5% in the MI patient cohort (carrier frequency 14.4%), compared to 4.0% (carrier frequency 7.8%) in controls, conferring a relative risk of 1.95 (Table 23). This haplotype remained significant after adjusting for all haplotypes tested, using 1000 randomisation steps, with an adjusted P value=0.046. No other SNP haplotype had an adjusted P value less than 0.05. The two at-risk haplotypes A4 and HapB appear to be mutually exclusive with no instance where the same chromosome carries both haplotypes.
Discussion:

These results show that variants of the gene encoding FLAP associate with increased risk of MI and stroke. In the Icelandic cohort, a haplotype that spans the FLAP gene is carried by 30% of all MI patients and almost doubles the risk of MI. These findings were subsequently replicated in an independent cohort of stroke patients. In addition, another haplotype that spans the FLAP gene is associated with MI in a British cohort. Suggestive linkage to chromosome 13q12 was observed with several different phenotypes, including female MI, early onset MI of both sexes, and ischemic stroke or TIA in males. However, surprisingly, the strongest haplotype association was observed to males with MI or stroke. Therefore, there may be other variants or haplotypes within the FLAP gene, or in other genes within the linkage region, that also may confer risk to these cardiovascular phenotypes.

These data also show that the at-risk haplotype of the FLAP gene has increased frequency in all subgroups of stroke, including ischemic, TIA, and hemorrhagic stroke. Of interest is that The A4 haplotype confers significantly higher risk of MI and stroke than it does of PAOD. This could be explained by differences in the pathogenesis of these diseases. Unlike PAOD patients who have ischemic legs because of atherosclerotic lesions that are responsible for gradually diminishing blood flow to the legs, the MI and stroke patients have suffered acute events, with disruption of the vessel wall suddenly decreasing blood flow to regions of the heart and the brain.

Association was not found between The A4 haplotype and MI in a British cohort. However, significant association to MI was found with a different variant over the FLAP gene. The fact that different haplotypes of the gene are found conferring risk to MI in a second population is not surprising. A common disease like MI associates with many different mutations or sequence variations, and the frequencies of these disease associated variants may differ between populations. Furthermore, the same mutations may be seen arising on different haplotypic backgrounds.

Markers Utilized Herein

TABLE 24

Position (Mb) of microsatellite markers sequence assembly (SA5),
primers and size of the markers.

| | | | | |
|---|---|---|---|---|
| mb | Marker | Forward | Reverse | size |

| | | | | |
|---|---|---|---|---|
| 25.0920 | DG13S2101 | ACGGTGATGACGCCTACATT | TCACATGGACCAATTACCTAGA | 188 |
| 42 | | (SEQ ID NO: 4) | A | |
| | | | (SEQ ID NO: 5) | |
| 25.0920 | DG13S48 | CAAATTTCAGATGTGCCAACC | ACGGTGATGACGCCTACATT | 214 |
| 42 | | (SEQ ID NO: 6) | (SEQ ID NO: 7) | |
| 25.3965 | D13S1304 | ACCAGCCTTTGCTTAGGA | ACATTCTAGTGCTACAGGGTAC | 133 |
| 04 | | (SEQ ID NO: 8) | TC | |
| | | | (SEQ ID NO: 9) | |
| 25.3965 | DG13S2105 | TGTTCTGCACACGAACATTCT | TCCTGAGTCCTCTCCACCTG | 104 |
| 35 | | (SEQ ID NO: 10) | (SEQ ID NO: 11) | |
| 25.4455 | DG13S2106 | TGGGAATTAATGAAGAACAACAA | CATGTTTCGAAGAACTCAAGAG | 428 |
| 11 | | A | G | |
| | | (SEQ ID NO: 12) | (SEQ ID NO: 13) | |
| 25.5449 | D13S1254 | AAATTACTTCATCTTGACGATAAC | CTATTGGGGACTGCAGAGAG | 218 |
| 20 | | A | (SEQ ID NO: 15) | |
| | | (SEQ ID NO: 14) | | |
| 25.5449 | DG13S2107 | GGGACTGCAGAGAGCAGAAG | CAAGAAGGGAAATTCCTACGC | 95 |
| 25 | | (SEQ ID NO: 16) | (SEQ ID NO: 17) | |
| 25.5659 | DG13S55 | AGCCAGTGTCCACAAGGAAG | GAGGGTGAGACACATCTCTGG | 283 |
| 56 | | (SEQ ID NO: 18) | (SEQ ID NO: 19) | |
| 25.6057 | DG13S54 | AATCGTGCCTCAGTTCCATC | CCACCAGGAACAACACACAC | 156 |
| 93 | | (SEQ ID NO: 20) | (SEQ ID NO: 21) | |
| 25.6196 | D13S625 | TTGCTCTCCAGCCTGGGC | TTCCTCTGGCTGCCTGCG | 185 |
| 93 | | (SEQ ID NO: 22) | (SEQ ID NO: 23) | |
| 25.6874 | DG13S1479 | TTTGATTCCGTGGTCCATTA | TTATTTGGTCGGTGCACCTTT | 339 |
| 22 | | (SEQ ID NO: 24) | (SEQ ID NO: 25) | |
| 25.7493 | DG13S1440 | GGTAGGTTGAAATGGGCTAACA | TCATGACAAGGTGTTGGATTT | 153 |
| 44 | | (SEQ ID NO: 26) | (SEQ ID NO: 27) | |
| 25.9012 | DG13S1890 | CCTCCTCTGCCATGAAGCTA | CTATTTGGTCTGCGGGTTGT | 418 |
| 12 | | (SEQ ID NO: 28) | (SEQ ID NO: 29) | |
| 25.9280 | DG13S1879 | TTTGAGCCCAGATCTAAGCAA | AAATGTTAATGTCACCGACAAA | 443 |
| 81 | | (SEQ ID NO: 30) | (SEQ ID NO: 31) | |
| 25.9326 | DG13S1540 | TACTGGGTTATCGCCTGACC | CCAATGGACCTCTTGGACAT | 152 |
| 09 | | (SEQ ID NO: 32) | (SEQ ID NO: 33) | |
| 25.9467 | DG13S1889 | TTTGAATGTTCATATATTTGTGGT | CCCTCGTAATGAAACCTATTTG | 222 |
| 43 | | G | A | |
| | | (SEQ ID NO: 34) | (SEQ ID NO: 35) | |
| 25.9486 | DG13S59 | TTTCGGCACAGTCCTCAATA | CAGGGTGTGGTGACAT | 228 |
| 79 | | (SEQ ID NO: 36) | (SEQ ID NO: 37) | |
| 25.9523 | DG13S1894 | TGTTTCTTTCTTTCTCTCTCTCTTT | AAATGAGTTCAATGAGTTGTGG | 209 |
| 47 | | C | TT | |
| | | (SEQ ID NO: 38) | (SEQ ID NO: 39) | |
| 25.9883 | DG13S1545 | CAGAGAGGAACAGGCAGAGG | AGTGGCTGGGAAGCCTTATT | 394 |
| 60 | | (SEQ ID NO: 40) | (SEQ ID NO: 41) | |
| 26.0718 | DG13S1524 | AGGTGAGAGAACAAACCTGTCTT | GCCTTCCTTCTAAGGCCAAC | 115 |
| 66 | | (SEQ ID NO: 42) | (SEQ ID NO: 43) | |
| 26.1834 | DG13S1491 | TGTTATACATTTCAATTTCACCTC | GTACTCCAGCCGGGCAAC | 286 |
| 92 | | A | (SEQ ID NO: 45) | |
| | | (SEQ ID NO: 44) | | |
| 26.2362 | DG13S62 | TTGTTCAGTGCTCTATAGTTACAA | GGTCACAAGCTATGCGATTA | 158 |
| 89 | | AGT | (SEQ ID NO: 47) | |
| | | (SEQ ID NO: 46) | | |
| 26.2734 | D13S1244 | TCAACAAGTGGATTAAGAAACTG | CTGTTTATGGCTGAGAAGTATG | 86 |
| 63 | | TG | C | |
| | | (SEQ ID NO: 48) | (SEQ ID NO: 49) | |
| 26.2869 | DG13S64 | TAGCAGGGTGCAGTCTA | ACCATACCACCACCACCATC | 247 |
| 35 | | (SEQ ID NO: 50) | (SEQ ID NO: 51) | |
| 26.3145 | D13S243 | ACTGTACTTCTGCCTGGGC | TTTTGTAATGCCTCAACCATG | 147 |
| 01 | | (SEQ ID NO: 52) | (SEQ ID NO: 53) | |
| 26.3271 | DG13S1529 | CTGTAGACTTTATCCCTGACTTAC | CAATGAATGATGAAGATTCCAC | 132 |
| 84 | | TG | TC | |
| | | (SEQ ID NO: 54) | (SEQ ID NO: 55) | |
| 26.3387 | DG13S1908 | TGACACCATGTCTTACTGTTTGC | GAGGATACAATGAGAACCAAAT | 224 |
| 67 | | (SEQ ID NO: 56) | CTC | |
| | | | (SEQ ID NO: 57) | |
| 26.3880 | DG13S1546 | CCACAGAATGCTCCAAAGGT | GAGTTCAAGTGATGGATGACG | 357 |
| 34 | | (SEQ ID NO: 58) | A | |
| | | | (SEQ ID NO: 59) | |
| 26.4358 | DG13S1444 | CAGATAGATGAATAGGTGGATGG | CACTGTTCCAAGTGCTTTGC | 193 |
| 11 | | A | (SEQ ID NO: 61) | |
| | | (SEQ ID NO: 60) | | |
| 26.4866 | DG13S1458 | GCAGGGCAAACTGCCTTAT | TTTGGTGAAATGTCTGTTTATG | 402 |
| 57 | | (SEQ ID NO: 62) | G | |
| | | | (SEQ ID NO: 63) | |
| 26.5045 | D13S252 | CTCAACCTGGCTTCTACT | TACTCCTTAATAAACTCCCC | 338 |
| 45 | | (SEQ ID NO: 64) | (SEQ ID NO: 65) | |
| 26.5082 | DG13S66 | TATGCGTTGTGTGTGTG | GGGCCTTAGATTCTTGTAGTG | 217 |
| 31 | | (SEQ ID NO: 66) | G | |
| | | | (SEQ ID NO: 67) | |
| 27.1151 | DG13S1554 | CTCGCATCTCGCTTCTCACT | CTCAAGGGTCCAGTGGTTTG | 420 |
| 20 | | (SEQ ID NO: 68) | (SEQ ID NO: 69) | |
| 27.1406 | DG13S1907 | TGTCCAGACTGCCTCCTACA | TGCAACACCTGGTTCACAAT | 131 |
| 75 | | (SEQ ID NO: 70) | (SEQ ID NO: 71) | |
| 27.1458 | D13S802 | CACAGTGAGACTCTATCTCAAAA | TCAGACTGGCTTAGACTGTGG | 150 |
| 42 | | A | (SEQ ID NO: 73) | |
| | | (SEQ ID NO: 72) | | |
| 27.2406 | DG13S1892 | AAATTCCAAAGGCCAGGTG | CCATACAGTTTCCTAGGTTCTG | 373 |
| 16 | | (SEQ ID NO: 74) | G | |
| | | | (SEQ ID NO: 75) | |
| 27.2534 | DG13S1849 | CACCTGGCCAAATGTTTGTT | TGCTTGAATCCAGAGACTGC | 190 |
| 52 | | (SEQ ID NO: 76) | (SEQ ID NO: 77) | |
| 27.2738 | DG13S68 | TTTGCGAGTCCTTGTGGAGT | ACAGTCCGCTCCCTCCTAAT | 238 |
| 60 | | (SEQ ID NO: 78) | (SEQ ID NO: 79) | |
| 27.2804 | DG13S69 | ATGCTTGGCCCTCAGTTT | TTGGCAACCCAAGCTAATATG | 296 |
| 61 | | (SEQ ID NO: 80) | (SEQ ID NO: 81) | |
| 27.4837 | D13S1250 | CTCCACAGTGACAGTGAGG | GAGAGGTTCCCAATCCC | 160 |
| 99 | | (SEQ ID NO: 82) | (SEQ ID NO: 83) | |
| 27.6104 | D13S1448 | CATCAACCTCCCCACCAC | TATTTTTTCAGTCCCACAGTTA | 227 |
| 06 | | (SEQ ID NO: 84) | GC | |
| | | | (SEQ ID NO: 85) | |
| 27.6158 | DG13S574 | CAGCTCCTGGCCATATTTCT | GAGCCATTTCTCTGGGTCTG | 153 |
| 14 | | (SEQ ID NO: 86) | (SEQ ID NO: 87) | |
| 27.6412 | DG13S73 | GGTCCGTGTCAACCCTTAGA | CAGGTTGATGGGAGGGAAA | 198 |
| 11 | | (SEQ ID NO: 88) | (SEQ ID NO: 89) | |
| 27.6615 | DG13S1532 | CGGGAAATGACAGTGAGACC | TGCCTAGATTCTCCCGTAAG | 163 |
| 07 | | (SEQ ID NO: 90) | (SEQ ID NO: 91) | |
| 27.7053 | D13S1242 | GTGCCCAGCCAGATTC | GCCCCCAGTCAGGTTT | 198 |
| 47 | | (SEQ ID NO: 92) | (SEQ ID NO: 93) | |
| 27.8838 | DG13S576 | TTTCTCTCTCCACGGAATGAA | AACCCATTCTCACAGGGTGTA | 199 |
| 72 | | (SEQ ID NO: 94) | (SEQ ID NO: 95) | |
| 27.8973 | DG13S1917 | AGGAGTGTGGCAGCTTTGAG | TGGATTCCCGTGAGTACCAG | 165 |
| 65 | | (SEQ ID NO: 96) | (SEQ ID NO: 97) | |
| 27.9321 | D13S217 | ATGCTGGGATCACAGGC | AACCTGGTGGACTTTTGCT | 170 |
| 54 | | (SEQ ID NO: 98) | (SEQ ID NO: 99) | |
| 28.0806 | DG13S581 | AGCATTTCCAATGGTGCTTT | CATGTTGATATGCCTGAAGGA | 367 |
| 32 | | (SEQ ID NO: 100) | (SEQ ID NO: 101) | |
| 28.1653 | DG13S1471 | CACTGTCTGCTGCCACTCAT | AGAGATTATGTGATGTACCCTC | 267 |
| 48 | | (SEQ ID NO: 102) | TCTAT | |
| | | | (SEQ ID NO: 103) | |
| 28.3032 | DG13S583 | CAAGCCTGGGACACAGAAAT | TTTGCAGACACCACAACACA | 264 |
| 52 | | (SEQ ID NO: 104) | (SEQ ID NO: 105) | |
| 28.3032 | D13S120 | ATGACCTAGAAATGATACTGGC | CAGACACCACAACACACATT | 175 |
| 56 | | (SEQ ID NO: 106) | (SEQ ID NO: 107) | |
| 28.3855 | D13S1486 | TGGTTTAAAAACCTCATGCC | ATCCCAAACTCTGTACTTATGT | 151 |
| 66 | | (SEQ ID NO: 108) | AGG | |
| | | | (SEQ ID NO: 109) | |
| 28.4155 | DG13S1024 | TTTGCACATACACATAAGCGAAC | CACAAATCCCGTGCACTAAA | 139 |
| 30 | | (SEQ ID NO: 110) | (SEQ ID NO: 111) | |
| 28.4155 | DG13S1510 | ATTCCTGGGCTCATGGTACA | TGCCGTCATCTGCTTTAGAA | 390 |
| 30 | | (SEQ ID NO: 112) | (SEQ ID NO: 113) | |
| 28.4303 | DG13S1495 | CCTTGGCTGTTGTGACTGGT | CACTCAGGTGGGAGGATCAC | 285 |
| 08 | | (SEQ ID NO: 114) | (SEQ ID NO: 115) | |
| 28.5175 | DG13S1482 | GCTGTTTCCTTGGCTTCTTCT | CCCATACTTGAGATGACCATGA | 291 |
| 41 | | (SEQ ID NO: 116) | (SEQ ID NO: 117) | |
| 28.5510 | DG13S1845 | CACTTTGCCAGTAGCCTTGA | TTGGGAAAGTTAACCCAGAGA | 284 |
| 60 | | (SEQ ID NO: 118) | (SEQ ID NO: 119) | |
| 28.6349 | DG13S1030 | TTTGGGAAGAGCCATGAGAC | CTCTGGGCATTGGAGGATTA | 354 |
| 03 | | (SEQ ID NO: 120) | (SEQ ID NO: 121) | |
| 28.6349 | DG13S1467 | TTTGGGAAGAGCCATGAGAC | AATGCCCATGTGCACTGTAG | 231 |
| 03 | | (SEQ ID NO: 122) | (SEQ ID NO: 123) | |
| 28.6866 | DG13S584 | GGGAGACAAGTCAGGTGAGG | CTGAGTATGGAGTCTTCATCAT | 151 |
| 07 | | (SEQ ID NO: 124) | TATC | |
| | | | (SEQ ID NO: 125) | |
| 28.7940 | DG13S1519 | TCGTCTCGAAGAAAGAAAGAAGA | CACCATGGGTTAATTGCACA | 286 |
| 32 | | (SEQ ID NO: 126) | (SEQ ID NO: 127) | |
| 28.8761 | DG13S77 | TGACGTGGGTTCAGGTTGTA | AGTGCATTGGTGCCTTCTCT | 220 |
| 56 | | (SEQ ID NO: 128) | (SEQ ID NO: 129) | |
| 28.9707 | DG13S586 | GGACTGCCAATTCTACAGCA | TTTCCATGGGAAATTTGGTC | 151 |
| 23 | | (SEQ ID NO: 130) | (SEQ ID NO: 131) | |
| 28.9756 | DG13S79 | TGCTACTAGATTTGACCAACCA | GACTTGTAAAGGATTTAGTGAT | 128 |
| 41 | | (SEQ ID NO: 132) | TTCG | |
| | | | (SEQ ID NO: 133) | |
| 29.0593 | DG13S80 | GTGGAAGGCCTCTCTTG | TGCTTCTTGAGGGAAAGCAT | 233 |
| 94 | | (SEQ ID NO: 134) | (SEQ ID NO: 135) | |
| 29.1261 | DG13S82 | CACGTGGTTCACCTCTCTAGG | TTGGCCACTTATTTGTG | 302 |
| 52 | | (SEQ ID NO: 136) | (SEQ ID NO: 137) | |
| 29.1546 | D13S1299 | CGATGAGTGACAGGGCT | CCTCGTGGGTGGAATAA | 225 |
| 91 | | (SEQ ID NO: 138) | (SEQ ID NO: 139) | |
| 29.1547 | DG13S85 | TTGGCCATTAGCAATTAGCA | CGTGGGTGGAATAAATCAGG | 153 |
| 37 | | (SEQ ID NO: 140) | (SEQ ID NO: 141) | |
| 29.1584 | D13S629 | GTTGAGGCAAGAGAATCACT | GCACATTTACACCAGGGTG | 145 |
| 62 | | (SEQ ID NO: 142) | (SEQ ID NO: 143) | |
| 29.2240 | DG13S1934 | CCTTCAGAGGATTTCCCTTTC | CTGGTTTGACTCCAGCTTCA | 431 |
| 60 | | (SEQ ID NO: 144) | (SEQ ID NO: 145) | |
| 29.2454 | DG13S1098 | TGTTCAAACCTAAGGTGCTTCA | GAAACAACAACAACAACAACAA | 416 |
| 62 | | (SEQ ID NO: 146) | CA | |
| | | | (SEQ ID NO: 147) | |
| 29.2598 | DG13S1104 | CCTGGCACGGAATAGACACT | GGCCTCCTTTGCTCTGAAG | 378 |
| 40 | | (SEQ ID NO: 148) | (SEQ ID NO: 149) | |
| 29.2944 | DG13S1097 | CATCCCTGTGGCTGATTAAGA | AACAGTTCCAGCCCGTTCTA | 162 |
| 36 | | (SEQ ID NO: 150) | (SEQ ID NO: 151) | |
| 29.3097 | DG13S1110 | TTTCAAAGGAATATCCAAGTGC | TGGCGTACCATATAAACAGTTC | 265 |
| 00 | | (SEQ ID NO: 152) | TC | |
| | | | (SEQ ID NO: 153) | |
| 29.3099 | DG13S86 | TTTCAAAGGAATATCCAAGTGC | AAACGTGACACTTCCACACA | 177 |
| 09 | | (SEQ ID NO: 154) | (SEQ ID NO: 155) | |
| 29.3599 | DG13S87 | TTCAATGAAGGTGCCGAAGT | TGTCTATCCCAAAGCAA | 218 |
| 61 | | (SEQ ID NO: 156) | (SEQ ID NO: 157) | |
| 29.5224 | DG13S1111 | GCAAGACTCTGTTGAAGAAGAAG | TCCCTCTGTTTGAGTTTCTCG | 110 |
| 43 | | A | (SEQ ID NO: 159) | |
| | | (SEQ ID NO: 158) | | |
| 29.5746 | DG13S1101 | AGGCACAGTCGCTCATGTC | AAACTTTAGCTAATGGTGGTCA | 333 |
| 65 | | (SEQ ID NO: 160) | AA | |
| | | | (SEQ ID NO: 161) | |
| 29.6227 | DG13S1106 | TGTGATTCCAGGGAGCTATCA | TAGGTGTGTGGAGGACAGCA | 416 |
| 55 | | (SEQ ID NO: 162) | (SEQ ID NO: 163) | |
| 29.6589 | DG13S172 | CCAGTTTCAGTTAGCCAAGTCTG | GAGAGGGAATGAATGCAGGA | 267 |
| 10 | | (SEQ ID NO: 164) | (SEQ ID NO: 165) | |
| 29.6657 | D13S1246 | GAGCATGTGTGACTTTCATATTC | AGTGGCTATTCATTGCTACAGG | 177 |
| 09 | | AG | (SEQ ID NO: 167) | |
| | | (SEQ ID NO: 166) | | |
| 29.6725 | DG13S1103 | TTGCTGGATGCTGGTTTCTA | AAAGAGAGAGAGAAAGAGAAA | 264 |
| 61 | | (SEQ ID NO: 168) | GAAAGA | |
| | | | (SEQ ID NO: 169) | |
| 29.8259 | D13S289 | CTGGTTGAGCGGCATT | TGCAGCCTGGATGACA | 260 |
| 75 | | (SEQ ID NO: 170) | (SEQ ID NO: 171) | |
| 29.8266 | DG13S166 | CCTATGGAAGCATAGGGAAGAA | CCCACTTCTGAGTCTCCTGAT | 395 |
| 31 | | (SEQ ID NO: 172) | (SEQ ID NO: 173) | |
| 29.9066 | DG13S164 | GGGATGCAGAAAGGATGTGT | AAGAATGCTGGCCAACGTAA | 218 |
| 89 | | (SEQ ID NO: 174) | (SEQ ID NO: 177) | |
| 29.9067 | D13S1238 | CTCTCAGCAGGCATCCA | GCCAACGTAATTGACACCA | 129 |
| 00 | | (SEQ ID NO: 178) | (SEQ ID NO: 179) | |
| 30.0313 | D13S290 | CCTTAGGCCCCATAATCT | CAAATTCCTCAATTGCAAAAT | 176 |
| 78 | | (SEQ ID NO: 180) | (SEQ ID NO: 181) | |
| 30.0863 | D13S1229 | GGTCATTCAGGGAGCCATTC | CCATTATATTTCACCAAGAGGC | 119 |
| 03 | | (SEQ ID NO: 182) | TGC | |
| | | | (SEQ ID NO: 183) | |
| 30.1928 | DG13S1460 | TGCCTGGTCATCTACCCATT | TCTACTGCAGCGCTGATCTT | 264 |
| 47 | | (SEQ ID NO: 184) | (SEQ ID NO: 185) | |
| 30.2176 | DG13S1933 | CATTTATGAATGGAGGTGAAGC | ATGGGAGCTCAAAGGGAAAT | 186 |
| 70 | | (SEQ ID NO: 186) | (SEQ ID NO: 187) | |
| 30.3032 | DG13S1448 | CAGCAGGAAGATGGACAGGT | CACACTGCATCACACATACCC | 136 |
| 13 | | (SEQ ID NO: 188) | (SEQ ID NO: 189) | |
| 30.3178 | D13S1287 | TATGCCAGTATGCCTGCT | GTCACATCAGTCCATTTGC | 232 |
| 71 | | (SEQ ID NO: 190) | (SEQ ID NO: 191) | |
| 30.3421 | DG13S1061 | CCAAAGCAAGTAACCTCCTCA | AAACAATCACTGCCCTCTGG | 227 |
| 02 | | (SEQ ID NO: 192) | (SEQ ID NO: 193) | |
| 30.5718 | DG13S1904 | TGATGAAATTGCCTAGTGATGC | GGATCCAATCGTACGCTACC | 136 |
| 37 | | (SEQ ID NO: 194) | (SEQ ID NO: 195) | |
| 30.6434 | DG13S882 | CGAATGGGTGACTAACAGCA | CTGGAGTGCAGGGACATGA | 378 |
| 38 | | (SEQ ID NO: 196) | (SEQ ID NO: 197) | |
| 30.6659 | DG13S295 | AAAGAAATATTCCAAGAAGAAAG | TTGCACAACTTTGTGTAGAGCA | 279 |
| 37 | | AAA | T | |
| | | (SEQ ID NO: 198) | (SEQ ID NO: 199) | |
| 30.6744 | D13S1226 | GGGTATGTCTTTATTCTCGGCAG | GTGCATTCACAGACCAGTCATT | 219 |
| 68 | | TA | (SEQ ID NO: 201) | |
| | | (SEQ ID NO: 200) | | |
| 30.6909 | DG13S293 | GGGCTTGAAGGCACTAAATGT | CCAAGCAGTAATTCCTTCCTCA | 313 |
| 59 | | (SEQ ID NO: 202) | (SEQ ID NO: 203) | |
| 30.7124 | DG13S1490 | ACCTAAACACCACGGACTGG | CAGGTATCGACATTCTTCCAAA | 418 |
| 68 | | (SEQ ID NO: 204) | (SEQ ID NO: 205) | |
| 30.8244 | DG13S93 | TGGGAAGCCAGTAAAGTAGGAA | AAAGAGACTCCACACATCCATT | 190 |
| 83 | | (SEQ ID NO: 206) | T | |
| | | | (SEQ ID NO: 207) | |
| 30.8248 | DG13S94 | AGGGCTATTCCTCAAGGTGTT | TGCTAACACTACCCTCGCAAT | 332 |
| 59 | | (SEQ ID NO: 208) | (SEQ ID NO: 209) | |
| 30.9284 | DG13S1534 | GGGCAGGAATCTCTGAAGTG | CTCCACTGAGAAGCCAAGGA | 382 |
| 29 | | (SEQ ID NO: 210) | (SEQ ID NO: 211) | |
| 30.9403 | DG13S95 | AGGCCAAGCTGGTCCATAG | TCTCTCAAAGCCTCGCTCTC | 126 |
| 69 | | (SEQ ID NO: 212) | (SEQ ID NO: 213) | |
| 30.9702 | DG13S96 | CCTTTGAGGCTGGATCTGTT | TTTCCTTATCATTCATTCCCTCA | 218 |
| 38 | | (SEQ ID NO: 214) | (SEQ ID NO: 215) | |
| 31.0388 | D13S260 | AGATATTGTCTCCGTTCCATGA | CCCAGATATAAGGACCTGGCT | 163 |
| 74 | | (SEQ ID NO: 216) | A | |
| | | | (SEQ ID NO: 217) | |
| 31.0922 | DG13S17 | TTTAAGCCCTGTGGAATGTATTT | GACATTGCAGGTCAAGTAGGG | 157 |
| 94 | | (SEQ ID NO: 218) | (SEQ ID NO: 219) | |
| 31.2078 | DG13S306 | TGCATAAGGCTGGAGACAGA | CACAGCAGATGGGAGCAAA | 158 |
| 44 | | (SEQ ID NO: 220) | (SEQ ID NO: 221) | |
| 31.2605 | DG13S18 | GTGCATGTGCATACCAGACC | GGCAAGATGACCTCTGGAAA | 319 |
| 21 | | (SEQ ID NO: 222) | (SEQ ID NO: 223) | |
| 31.2997 | DG13S1905 | GTCCACTGCAGCACACAGAG | GCACTGGTAGATACATGCTAAC | 383 |
| 20 | | (SEQ ID NO: 224) | G | |
| | | | (SEQ ID NO: 225) | |
| 31.3532 | DG13S307 | GGGTATCTTGGCCAGGTGT | TGGCTAAGCACAATCCCTTT | 403 |
| 30 | | (SEQ ID NO: 226) | (SEQ ID NO: 227) | |
| 31.3551 | DG13S1062 | TTTGTGTTCCAGGTGAGAATTG | GAACCATATCCCAAGGCACT | 120 |
| 35 | | (SEQ ID NO: 228) | (SEQ ID NO: 229) | |
| 31.4143 | DG13S1874 | AACCCAAATCAACAAACCAGA | AATGAATTCTGGGTCACATGC | 404 |
| 29 | | (SEQ ID NO: 230) | (SEQ ID NO: 231) | |
| 31.4295 | DG13S1093 | TTGTTCCCACATTCATTCTACA | TTAAACTCGTGGCAAAGACG | 273 |
| 62 | | (SEQ ID NO: 232) | (SEQ ID NO: 233) | |
| 31.6265 | DG13S1059 | CACCATGCCTGGCTCTTT | AACTTCTCCAGTTGTGTGGTTG | 330 |
| 02 | | (SEQ ID NO: 234) | (SEQ ID NO: 235) | |
| 31.7237 | DG13S1086 | AGCTGAGCTCATGCCACT | CAAGACCTTGTGCATTTGGA | 155 |
| 49 | | (SEQ ID NO: 236) | (SEQ ID NO: 237) | |
| 31.7460 | DG13S1515 | AGCCAGACATGGTAGTGTGC | GCAATAACTCACACATCAGCAA | 417 |
| 74 | | (SEQ ID NO: 238) | (SEQ ID NO: 239) | |
| 31.8557 | D13S171 | CCTACCATTGACACTCTCAG | TAGGGCCATCCATTCT | 231 |
| 32 | | (SEQ ID NO: 240) | (SEQ ID NO: 241) | |
| 31.9173 | DG13S1092 | ACCAAGATATGAAGGCCAAA | CCTCCAGCTAGAACAATGTGAA | 176 |
| 32 | | (SEQ ID NO: 242) | (SEQ ID NO: 243) | |
| 32.0028 | DG13S1449 | TGTCCATAGCTGTAGCCCTGT | CTCAATGGGCATCTTTAGGC | 279 |
| 52 | | (SEQ ID NO: 244) | (SEQ ID NO: 245) | |
| 32.0729 | DG13S1489 | TGTAATTCAACGACTGGTGTCC | AGCTTCTGATGGTTGCTGGT | 130 |
| 57 | | (SEQ ID NO: 246) | (SEQ ID NO: 247) | |
| 32.0839 | DG13S312 | CAAACAAACAAACAAGCAAACC | TGGACGTTTCTTTCAGTGAGG | 349 |
| 89 | | (SEQ ID NO: 248) | (SEQ ID NO: 249) | |
| 32.1251 | DG13S1511 | TGATAACTTACCAGCATGTGAGC | TCACCTCACCTAAGGATCTGC | 314 |
| 77 | | (SEQ ID NO: 250) | (SEQ ID NO: 251) | |
| 32.1835 | DG13S314 | CATGCAATTGCCCAATAGAG | TTGGGCTTGTCTACCTAGTTCA | 335 |
| 47 | | (SEQ ID NO: 252) | (SEQ ID NO: 253) | |
| 32.1953 | DG13S1090 | TGGGTTCCTCATACTGGAGTG | GCCTGAGCTCCAAGCTCTTT | 169 |
| 58 | | (SEQ ID NO: 254) | (SEQ ID NO: 255) | |
| 32.2510 | DG13S1071 | GCTGCACGTATTTGTTGGTG | AAACAGCAGAAATGGGAACC | 239 |
| 38 | | (SEQ ID NO: 256) | (SEQ ID NO: 257) | |
| 32.3568 | DG13S1068 | CCGTGGGCTATCAATTTCTG | AAGATGCAATCTGGTTTCCAA | 238 |
| 95 | | (SEQ ID NO: 258) | (SEQ ID NO: 259) | |
| 32.3730 | DG13S1077 | CCCAAGACTGAGGAGGTCAA | GCTGACGGAGAGGAAAGAGA | 374 |
| 40 | | (SEQ ID NO: 260) | (SEQ ID NO: 261) | |
| 32.4227 | DG13S1906 | TGACAAGGGTGTGGTTATGG | CCGCACTTTCTCTTCTGGAC | 425 |
| 80 | | (SEQ ID NO: 262) | (SEQ ID NO: 263) | |
| 32.5115 | DG13S316 | TGAGAAGCCTGGGCATTAAG | ACAAGCTCATCCAGGGAAAG | 243 |
| 90 | | (SEQ ID NO: 264) | (SEQ ID NO: 265) | |
| 32.6105 | DG13S317 | TTGGAAAGGAAGAAAGGAAGG | TTGAAACCTAAATGCCACCTG | 215 |
| 17 | | (SEQ ID NO: 266) | (SEQ ID NO: 267) | |
| 32.6107 | D13S1493 | ACCTGTTGTATGGCAGCAGT | GGTTGACTCTTTCCCCAACT | 248 |
| 13 | | (SEQ ID NO: 268) | (SEQ ID NO: 269) | |
| 32.7898 | DG13S1558 | AGAGCTGATCTGGCCGAAG | GGTGGACACAGAATCCACACT | 399 |
| 94 | | (SEQ ID NO: 270) | (SEQ ID NO: 271) | |
| 32.8659 | D13S267 | GGCCTGAAAGGTATCCTC | TCCCACCATAAGCACAAG | 160 |
| 50 | | (SEQ ID NO: 272) | (SEQ ID NO: 273) | |
| 32.9614 | DG13S1478 | TCAACCTAGGATTGGCATTACA | TCTAGGATTTGTGCCTTTCCA | 387 |
| 10 | | (SEQ ID NO: 274) | (SEQ ID NO: 275) | |
| 33.0099 | DG13S1513 | GACGTCTTAGGATTGACTTCTGC | CCAAATACACATTCTTAAAGGG | 173 |
| 22 | | (SEQ ID NO: 276) | AAA | |
| | | | (SEQ ID NO: 277) | |
| 33.1256 | DG13S1461 | GACTGCAGATCGTGGGACTT | TTCTCCAGAGAAACCAAACCA | 148 |
| 96 | | (SEQ ID NO: 278) | (SEQ ID NO: 279) | |
| 33.1684 | DG13S1551 | ATTCGTGCAGCTGTTTCTGC | GCATGACATTGTAAATGGAGG | 263 |
| 68 | | (SEQ ID NO: 280) | A | |
| | | | (SEQ ID NO: 281) | |
| 33.2549 | DG13S1884 | GGTGGGAATGTGTGACTGAA | CCAGGTACAACATTCTCCTGAT | 123 |
| 89 | | (SEQ ID NO: 282) | (SEQ ID NO: 283) | |
| 33.3401 | D13S1293 | TGCAGGTGGGAGTCAA | AAATAACAAGAAGTGACCTTCC | 129 |
| 24 | | (SEQ ID NO: 284) | TA | |
| | | | (SEQ ID NO: 285) | |
| 33.3469 | DG13S326 | TGTTCTCCTCACCCTGCTCT | TTTCAGGCTAGGAAGATCCTTT | 261 |
| 08 | | (SEQ ID NO: 286) | (SEQ ID NO: 287) | |
| 33.3926 | DG13S1518 | AAAGGATGCATTCGGTTAGAG | ACTGTCCTGTGCCTGTGCTT | 375 |
| 29 | | (SEQ ID NO: 288) | (SEQ ID NO: 289) | |
| 33.4055 | DG13S23 | CCTGAATAGGTGGAATTAAGATC | TCAAGGAGCATACACACACAC | 107 |
| 27 | | AA | A | |
| | | (SEQ ID NO: 290) | (SEQ ID NO: 291) | |
| 33.4315 | D13S620 | GTCCACCTAATGGCTCATTC | CAAGAAGCACTCATGTTTGTG | 185 |
| 36 | | (SEQ ID NO: 292) | (SEQ ID NO: 293) | |
| 33.4370 | DG13S1866 | AGCCTGTGATTGGCTGAGA | GGCTTACAGCTGCCTCCTTT | 410 |
| 92 | | (SEQ ID NO: 294) | (SEQ ID NO: 295) | |
| 33.4957 | DG13S1927 | CCCACAGAGCACTTTGTTAGA | GCCTCCCTTAAGCTGTTATGC | 401 |
| 18 | | (SEQ ID NO: 296) | (SEQ ID NO: 297) | |
| 33.5034 | DG13S1503 | CACTCTTTACTGCCAATCACTCC | GCCGTGTGGGTGTATGAAT | 226 |
| 40 | | (SEQ ID NO: 298) | (SEQ ID NO: 299) | |
| 33.5681 | DG13S332 | TTGTACCAGGAACCAAAGACAA | CACAGACAGAGGCACATTGA | 176 |
| 00 | | (SEQ ID NO: 300) | (SEQ ID NO: 301) | |
| 33.6758 | DG13S333 | GCTCTGGTCACTCCTGCTGT | CATGCCTGGCTGATTGTTT | 446 |
| 41 | | (SEQ ID NO: 302) | (SEQ ID NO: 303) | |
| 33.7713 | D13S220 | CCAACATCGGGAACTG | TGCATTCTTTAAGTCCATGTC | 191 |
| 89 | | (SEQ ID NO: 304) | (SEQ ID NO: 305) | |
| 33.8180 | DG13S1919 | CAGCAACTGACAACTCATCCA | CCTCAATCCTCAGCTCCMC | 255 |
| 41 | | (SEQ ID NO: 306) | (SEQ ID NO: 307) | |
| 33.8736 | DG13S1439 | TCCTTCACAGCTTCAAACTCA | AGTGAGAAGCTTCCATACTGGT | 239 |
| 14 | | (SEQ ID NO: 308) | (SEQ ID NO: 309) | |
| 33.9060 | DG13S335 | GCCAACCGTTAGACAAATGA | CTACATGTGCACCACAACACC | 201 |
| 65 | | (SEQ ID NO: 310) | (SEQ ID NO: 311) | |
| 33.9286 | DG13S340 | AGTTTATTGCCGCCGAGAG | ACCCACCACATTCACAAGC | 373 |
| 53 | | (SEQ ID NO: 312) | (SEQ ID NO: 313) | |
| 34.0194 | DG13S1496 | CGATTGCCATGTCTCTTTGA | GAGATCTGGCCTGGATTTGT | 155 |
| 55 | | (SEQ ID NO: 314) | (SEQ ID NO: 315) | |
| 34.0340 | DG13S342 | TGAGGCCAGCCTTACCTCTAT | CCAGACATGGTGGCTTGT | 366 |
| 89 | | (SEQ ID NO: 316) | (SEQ ID NO: 317) | |
| 34.0617 | DG13S344 | GAAGGAAGGAAGGGAAGGAA | AAGGATGAGAAGAGTCCATGC | 292 |
| 77 | | (SEQ ID NOv 318) | (SEQ ID NO: 319) | |
| 34.0672 | DG13S345 | AAATACCCTTTGAACAGACACAC | TAGCTGAGCATGGTGGTACG | 201 |
| 39 | | (SEQ ID NO: 320) | (SEQ ID NO: 321) | |
| 34.0778 | DG13S346 | AAAGACAAGACAGCAATCCAAA | GCAGAACCCAGGCTACAGAT | 152 |
| 74 | | (SEQ ID NO: 322) | (SEQ ID NO: 323) | |
| 34.0841 | DG13S347 | TCATTGTCAGCACAGAATGAACT | GGAGGGAGGGAAGAAAGAGA | 338 |
| 38 | | (SEQ ID NO: 324) | (SEQ ID NO: 325) | |
| 34.0843 | D13S624 | GCAACACAGTGAAAGCCCA | ACAGGAGCATGCCACCATG | 191 |
| 26 | | (SEQ ID NO: 326) | (SEQ ID NO: 327) | |
| 34.1560 | DG13S339 | GGGAAGAGGAGATTGACTTGTT | GGAACACCATCATTCCAACC | 232 |
| 75 | | (SEQ ID NO: 328) | (SEQ ID NO: 329) | |
| 34.1924 | DG13S1926 | TACAAGCTCCACCGTCCTTC | TGAGTTGCTGCCTCTTCAAA | 261 |
| 78 | | (SEQ ID NO: 330) | (SEQ ID NO: 331) | |
| 34.2202 | DG13S1469 | TGCTAATGGGCCAAGGAATA | GCTAATGTCCTCATGAATAGC | 382 |
| 27 | | (SEQ ID NO: 332) | C | |
| | | | (SEQ ID NO: 333) | |
| 34.3014 | DG13S351 | TGTCCTGCAGACAGATGGTC | CCTCCGGAGTAGCTGGATTA | 294 |
| 48 | | (SEQ ID NO: 334) | (SEQ ID NO: 335) | |
| 34.3878 | DG13S26 | GAGACTGGCCCTCATTCTTG | AAGAAGCCAGAGACAAAGAAA | 330 |
| 83 | | (SEQ ID NO: 336) | TACA | |
| | | | (SEQ ID NO: 337) | |
| 34.5354 | DG13S30 | CATCTATCTTTGGATTCAGTGGT | TGCTCCCAACATCTTACCAG | 388 |
| 41 | | G | (SEQ ID NO: 339) | |
| | | (SEQ ID NO: 338) | | |
| 34.5655 | DG13S1435 | TGTCCTCTGGTCATTTCTATGGT | CATGAATGAGAAGTGATGAATG | 235 |
| 94 | | (SEQ ID NO: 340) | G | |
| | | | (SEQ ID NO: 341) | |
| 34.6598 | DG13S1446 | AACACGGGAAATTCCAACAG | TGAAGAACTGAAATTGCCAGTA | 379 |
| 58 | | (SEQ ID NO: 342) | A | |
| | | | (SEQ IDNO: 343) | |
| 34.7122 | DG13S356 | CAGACACTGTAAACTGGCTTCG | GCCACATTGCTATCAGCGTA | 212 |
| 60 | | (SEQ ID NO: 344) | (SEQ ID NO: 345) | |
| 34.7387 | DG13S357 | TGTCATAGGCTTGCGGTATTT | TTGGTAGGGTCCTTTCCTTT | 202 |
| 56 | | (SEQ ID NO: 346) | (SEQ ID NO: 347) | |
| 34.7705 | DG13S1032 | GCCTGCTCACTGTTGTTTGA | CGGTTATCAGAGACTGGTGGT | 211 |
| 71 | | (SEQ ID NO: 348) | (SEQ ID NO: 349) | |
| 34.7996 | DG13S1557 | GGCTTATTTCATGTACGGCTA | GGTTAAACTCTACTTAGTCCTG | 158 |
| 79 | | (SEQ ID NO: 350) | ATGC | |
| | | | (SEQ ID NO: 351) | |
| 34.8829 | DG13S1925 | GAACTCTGCAGGCACCTCTT | CCTGAAGCGCTTGTACTGAA | 456 |
| 34 | | (SEQ ID NO: 352) | (SEQ ID NO: 353) | |
| 34.9326 | DG13S1484 | TGTTGCGTACTCAGCCCATA | GACAGGTGTCAAACGGGTCT | 246 |
| 90 | | (SEQ ID NO: 354) | (SEQ ID NO: 355) | |
| 34.9425 | DG13S360 | TTGGCTTCTCGCTCTTTCTT | AGCCATCAGTCACATGCAAA | 350 |
| 47 | | (SEQ ID NO: 356) | (SEQ ID NO: 357) | |
| 34.9989 | DG13S1522 | AGATCTCCAGGGCAGAGGAC | CCTTCCTCCCTCCTTCTCTC | 355 |
| 79 | | (SEQ ID NO: 358) | (SEQ ID NO: 359) | |
| 35.0749 | DG13S1517 | CGTCATTGATCCCAATCATCT | GGCTGATAGCCTCCCTTGTA | 235 |
| 62 | | (SEQ ID NO: 360) | (SEQ ID NO: 361) | |
| 35.0749 | DG13S1521 | GAGAGAGAGCAGCTTGCATGT | GGCTGATAGCCTCCCTTGTA | 172 |
| 62 | | (SEQ ID NO: 362) | (SEQ ID NO: 363) | |
| 35.1268 | DG13S364 | ACCTTTCAAGCTTCCGGTTT | TTCCATCCGTCCATCTATCC | 172 |
| 82 | | (SEQ ID NO: 364) | (SEQ ID NO: 365) | |
| 35.3286 | DG13S1036 | TTAAAGTCACTTGTCTGTGGTCA | TTTGTAGGAATCAAGTCAAATA | 216 |
| 63 | | (SEQ ID NO: 366) | ATGTA | |
| | | | (SEQ ID NO: 367) | |
| 35.3353 | DG13S367 | CAAACATCACACTGGGCAAA | TGCTTTGGAATCTTTCTTGCT | 301 |
| 64 | | (SEQ ID NO: 368) | (SEQ ID NO: 369) | |
| 35.3719 | DG13S1901 | CTGCCAGGATGTCAGCATT | TCCACACTTTCTCATCACCTAA | 440 |
| 57 | | (SEQ ID NO: 370) | A | |
| | | | (SEQ ID NO: 371) | |
| 35.4202 | DG13S1037 | CTTTCGGAAGCTTGAGCCTA | CCCAAGACCACTGCCATATT | 269 |
| 95 | | (SEQ ID NO: 372) | (SEQ ID NO: 373) | |
| 35.4258 | DG13S1854 | TGACAGGTTTGGGTATATTGGA | TGCTTAATGTAGTGGCAGCA | 124 |
| 41 | | (SEQ ID NO: 374) | (SEQ ID NO: 375) | |
| 35.5060 | DG13S1038 | TCCTGCCTTTGTGAATTCCT | GTTGAATGAGGTGGGCATTA | 334 |
| 53 | | (SEQ ID NO: 376) | (SEQ ID NO: 377) | |
| 35.5472 | DG13S1039 | CCATTTAATCCTCCAGCCATT | GCTCCACCTTGTTACCCTGA | 167 |
| 10 | | (SEQ ID NO: 378) | (SEQ ID NO: 379) | |
| 35.6092 | DG13S1840 | ACAACCCTGGAATCTGGACT | GAAGGAAAGGAAAGGAAAGAA | 217 |
| 52 | | (SEQ ID NO: 380) | A | |
| | | | (SEQ ID NO: 381) | |
| 35.6192 | DG13S369 | TGACAAGACTGAAACTTCATCAG | GATGCTTGCTTTGGGAGGTA | 257 |
| 86 | | (SEQ ID NO: 382) | (SEQ ID NO: 383) | |
| 35.6279 | D13S305 | TTGAGGACCTGTCGTTACG | TTATAGAGCAGTTAAGGCACA | 394 |
| 11 | | (SEQ ID NO: 384) | (SEQ ID NO: 385) | |
| 35.6566 | DG13S375 | TGAGGGTGGTAAGCCCTTATT | GGAGTTGTGGCCTCTCTCTCT | 192 |
| 59 | | (SEQ ID NO: 386) | (SEQ ID NO: 387) | |
| 35.7603 | D13S219 | AAGCAAATATGCAAAATTGC | TCCTTCTGTTTCTTGACTTAAC | 125 |
| 68 | | (SEQ ID NO: 388) | A | |
| | | | (SEQ ID NO: 389) | |
| 35.8258 | DG13S378 | TGCTAAGAGGGCAGATCTCA | GGCTCATAGCCAATTTCTCC | 324 |
| 52 | | (SEQ ID NO: 390) | (SEQ ID NO: 391) | |
| 35.8321 | DG13S32 | CGGCATTCTCAATAACCTCAA | TCTTTGATGAGGATCAATTAGT | 214 |
| 27 | | (SEQ ID NO: 392) | GG | |
| | | | (SEQ ID NO: 393) | |
| 35.8729 | DG13S1549 | ACGCACACACACACACACAC | TGCCTCTGTAATCCTGTGTAGC | 260 |
| 36 | | (SEQ ID NO: 394) | (SEQ ID NO: 395) | |
| 35.9123 | DG13S1473 | GCTCTAAGGTGGGTCCCAATA | GGGAATGACAAGATCAGTTTAC | 163 |
| 21 | | (SEQ ID NO: 396) | C | |
| | | | (SEQ ID NO: 397) | |

All references cited herein are incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1-205. (canceled)

206. A method of reducing C-reactive protein in a human subject, comprising:
selecting a human subject at risk for a disease or condition selected from the group consisting of myocardial infarction (MI), acute coronary syndrome (ACS), stroke, or peripheral arterial occlusive disease (PAOD); and
administering to the subject a composition comprising an agent that inhibits leukotriene synthesis in vivo, by inhibiting the activity of 5-Lipoxygenase activating protein (FLAP),
wherein the agent is administered in an amount effective to reduce serum C-reactive protein in the human subject, and
wherein the agent comprises a compound represented by the formula: 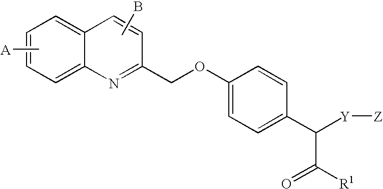
or pharmaceutically acceptable salt thereof,
wherein R′ represents a group of the formula: 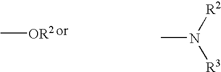
R² and R³ are identical or different and represent hydrogen, lower alkyl, phenyl, benzyl or a group of the formula: 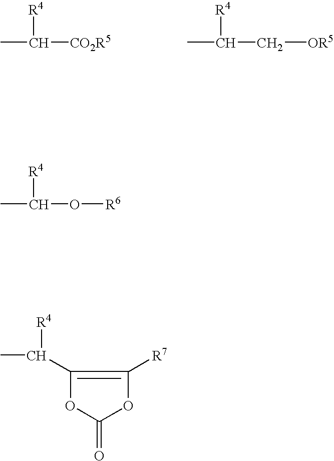
R⁴ represents hydrogen, lower alkyl, phenyl or benzyl, which can optionally be substituted by hydroxyl, carboxyl, lower alkoxycarbonyl, lower alkylthio, heteroaryl or carbamoyl, R⁵ represents hydrogen, lower alkyl, phenyl or benzyl, R⁶ represents a group of the formula —COR⁵ or —CO² R⁵, R⁷ represents hydrogen, lower alkyl or phenyl, Y represents a group of the formula: 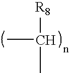
wherein R⁸ represents hydrogen, lower alkyl or phenyl and n denotes a number of 0 to 5, Z represents norbornyl, or represents a group of the formula: 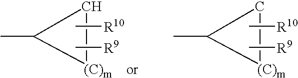
wherein R⁹ and R¹⁰ are identical or different and denote hydrogen, lower alkyl or phenyl, or R⁹ and R¹⁰ can together form a saturated carbocyclic ring having up to 6 carbon atoms and m denotes a number from 1 to 6, and A and B are identical or different and denote hydrogen, lower alkyl or halogen, or a pharmaceutically acceptable salt thereof.

207. A method according to claim 206, further comprising:
measuring serum C reactive protein in the human subject to monitor therapeutic efficacy of the agent, wherein a decrease in serum C-reactive protein following the administering of the agent indicates therapeutic efficacy.

208. A method according to claim 206, wherein the composition further comprises a physiologically acceptable carrier or excipient.

209. A method according to claim 206, wherein the selecting step comprises selecting a human subject susceptible to primary myocardial infarction.

210. A method according to claim 206, wherein the compound is selected from the group consisting of: 2-[4-(quinolin-2-yl-methoxy)phenyl]-2-cyclopentylacetic acid, 2-[4-(quinolin-2-yl-methoxy)phenyl]-2-cyclohexylacetic acid, and 2-[4-(quinolin-2-yl-methoxy)phenyl]-2-cycloheptylacetic acid, (+)-enantiomer of 2-[4-(quinolin-2-yl-methoxy)phenyl]-2-cyclopentylacetic acid, (−)-enantiomer of 2-[4-(quinolin-2-yl-methoxy)phenyl]-2-cyclopentylacetic acid, and pharmaceutically acceptable salts thereof.

211. A method according to claim 206, wherein the compound is BAY-X-1005.

212. A method according to claim 206, wherein the compound is BAY-X-1005 or a physiologically acceptable salt, formulation, or pro-drug thereof.

213. A method according to claim 212, wherein the composition is administered orally.

214. A method according to claim 206, further comprising monitoring at least one inflammatory marker in the human subject before and during the administering step, wherein the composition is administered to reduce the level of said inflammatory marker in the human subject.

215. A method according to claim 214, wherein the at least one inflammatory marker comprises myeloperoxidase (MPO).

216. A method according to claim 206, further comprising monitoring a leukotriene level in the subject before and during the administering step, wherein the composition is administered in an amount effective to reduce the leukotriene level in the subject.

217. A method according to claim 214 or 216, wherein the monitoring comprises monitoring in serum, plasma, or urine from the subject.

218. A method according to claim 206, wherein the selecting step comprises measuring at least one inflammatory marker selected from the group consisting of serum myeloperoxidase (MPO) and serum C reactive protein (CRP), and selecting a susceptible subject having elevated serum MPO or elevated serum CRP as being susceptible to MI.

219. A method according to claim 218, wherein the selecting step further comprises analyzing nucleic acid of a human subject for the presence or absence of at least one 5-lipoxygenase activating protein (FLAP) polymorphism that correlates with a susceptibility to myocardial infarction, and selecting a subject with the presence of at least one such polymorphism and with the presence of elevated serum CRP or MPO.

220. A method according to claim 206, wherein the selecting step comprises selecting a susceptible subject from at least one family or medical history risk factor selected from the group consisting of past or current smoker; diabetes; hypertension; serum total cholesterol >200mg/dL; elevated serum LDL cholesterol; low serum HDL cholesterol; elevated C-reactive protein (CRP); elevated serum amyloid A; hypercholesterolemia; elevated triglycerides; elevated lp(a); obesity; acute coronary syndrome (ACS); angina; atherosclerosis; ankle/brachial index less than 0.9; transient ischemic attack; transient monocular blindness; asymptomatic carotid stenosis; claudication; limb ischemia leading to gangrene, ulceration or amputation; and surgery to restore coronary artery blood flow.

221. A method according to claim 206, wherein the selecting step comprises measuring C-reactive protein (CRP) in serum of a human subject, wherein a subject with elevated CRP is identified as being at risk for MI.

222. A method according to claim 206, wherein the selecting comprises selecting a susceptible subject from measurements of serum CRP and serum low density lipoprotein cholesterol (LDL-C).

223. A method according to claim 206, wherein the selecting step comprises selecting a susceptible subject from an elevated measurement of at least one inflammatory marker selected from the group consisting of C-reactive protein (CRP), serum amyloid A, fibrinogen, interleukin-6, tissue necrosis factor-alpha (TNF-alpha), soluble vascular cell adhesion molecules (sVCAM), soluble intervascular adhesion molecules (sICAM), E-selectin, matrix metalloprotease type-1, matrix metalloprotease type-2, matrix metalloprotease type-3, matrix metalloprotease type-9, myeloperoxidase (MPO), and N-tyrosine.

224. A method according to claim 206, wherein the measurement is a measurement of myeloperoxidase.

225. A method according to claim 206, where the selecting comprises determining a FLAP genotype or haplotype of a human subject, and selecting for treatment a human subject with a FLAP genotype or haplotype that correlates with an increased risk of myocardial infarction.
